(19) United States
(12) Patent Application Publication
(10) Pub. No.: US 2023/0310537 A1
(43) Pub. Date: Oct. 5, 2023

(54) TREATMENT OF VASCULAR EHLERS-DANLOS SYNDROME AND RELATED SYNDROMES

(71) Applicant: The Johns Hopkins University, Baltimore, MD (US)

(72) Inventors: Harry C. Dietz, Towson, MD (US); Caitlin J. Bowen, Baltimore, MD (US)

(21) Appl. No.: 18/024,662

(22) PCT Filed: Sep. 3, 2021
(86) PCT No.: PCT/US21/49168

§ 371 Date: Mar. 3, 2023

Related U.S. Application Data
(60) Provisional application No. 63/075,047, filed on Sep. 4, 2020.

(51) Int. Cl. A61K 38/00; A61K 31/454; A61P 9/10

(52) U.S. Cl. CPC A61K 38/005; A61K 31/454; A61P 9/10

(57) ABSTRACT

Provided herein are, inter alia, method and compositions for treating vascular Ehlers-Danlos syndrome and related syndromes. Also included herein are kits for treating vascular Ehlers-Danlos syndrome and related syndromes.

### STATEMENT AS TO FEDERALLY SPONSORED RESEARCH

This invention was made with government support under Grant No. 07023, awarded by the Howard Hughes Medical Institute (HHMI); and under Grant No. 5R01AR041135, awarded by the National Institute of Arthritis and Musculoskeletal and Skin Diseases of the National Institute of Health. The Government has certain rights in the invention.

The present application claims the benefit of priority of U.S. provisional application no. 63/075,047 filed Sep. 4, 2020, which is incorporated herein by reference it its entirety.

### FIELD

The present invention relates to vascular Ehlers-Danlos syndrome (vEDS) and related syndromes.

### BACKGROUND

Vascular Ehlers Danlos Syndrome (vEDS) is an inherited connective tissue disorder caused by heterozygous mutations in the COL3A1 gene, resulting in spontaneous vascular and/or organ rupture. New compositions and methods for treating vEDS, and related syndromes are needed.

### BRIEF SUMMARY

Provided herein are, inter alia, methods, compositions and kits for treating and preventing vascular Ehlers-Danlos syndrome, and related syndromes. Also included herein are kits for treating vascular Ehlers-Danlos syndrome, and related syndromes.

In aspects, provided herein are methods of treating or preventing a vasculopathy in a subject. For example, the method includes administering an effective amount of an agent (e. g,. wherein the agent increases the activity or expression of a mitogen-activated protein kinase (MAPK), a protein phosphatase (PP), or a combination thereof), and thereby treating the vasculopathy.

In embodiments, the vasculopathy comprises vascular Ehlers-Danlos Syndrome (vEDS). In other embodiments, the vasculopathy may include related syndromes, including for example other presentations of aortic or peripheral aneurysms.

In embodiments, the agent of the present invention includes an antibody or fragment thereof, a polypeptide, a small molecule, a nucleic acid molecule, or any combination thereof.

As used herein, “small molecule” may be referred to broadly as an organic, inorganic or organometallic compound with a low molecular weight compound (e.g., a molecular weight of less than about 2,000 Da or less than about 1,000 Da). The small molecule may have a molecular weight of less than about 2,000 Da, a molecular weight of less than about 1,500 Da, a molecular weight of less than about 1,000 Da, a molecular weight of less than about 900 Da, a molecular weight of less than about 800 Da, a molecular weight of less than about 700 Da, a molecular weight of less than about 600 Da, a molecular weight of less than about 500 Da, a molecular weight of less than about 400 Da, a molecular weight of less than about 300 Da, a molecular weight of less than about 200 Da, a molecular weight of less than about 100 Da, or a molecular weight of less than about 50 Da. Small molecules are organic or inorganic. Exemplary organic small molecules include, but are not limited to, aliphatic hydrocarbons, alcohols, aldehydes, ketones, organic acids, esters, mono- and disaccharides, aromatic hydrocarbons, amino acids, and lipids. Exemplary inorganic small molecules comprise trace minerals, ions, free radicals, and metabolites. Alternatively, small molecules can be synthetically engineered to consist of a fragment, or small portion, or a longer amino acid chain to fill a binding pocket of an enzyme. Typically small molecules are less than one kilodalton.

In embodiments, an antibody described herein may be a polyclonal antisera or monoclonal antibody. The term antibody may include any of the various classes or sub-classes of immunoglobulin (e.g., IgG, IgA, IgM, IgD, or IgE derived from any animal, e.g., any of the animals conventionally used, e.g., sheep, rabbits, goats, or mice, or human), e.g., the antibody comprises a monoclonal antibody, e.g., a p38 monoclonal antibody.

In other embodiments, the MAPK comprises p38. For example, p38 includes an isoform selected from p38-α, p38-β, p38-γ, or p38-δ.

In cerain aspects, one or more p38 MAPK agonists may be administered to a patient in needed thereof in accordance with the present methods. Preferred p38 MAPK agonists include for example anismyin and sorbitol.

In other embodiments, the methods include administering an agent that decreases the activity or expression of extracellular signal-regulated kinase (ERK) or protein kinase C (PKC). In examples, the agent that decreases the activity or expression of ERK or PKC includes cobimetinib, trametinib, ruboxistaurin, enzastaurin, sotrastaurin, or any combination thereof.

In embodiments, the protein phosphatase (PP) includes protein phosphatase 1 (PP1), protein phosphatase 2A (PP2A), or a combination thereof.

In embodiments, the agent to increase the activity or expression of a mitogen-activated protein kinase (MAPK) (p38), a protein phosphatase (PP) (PP1), or a combination thereof) is a small molecule.

In embodiments, the effective amount of the agent to increase the activity or expression of p38 and/or PP1 is from about 0.001 mg/kg to 250 mg/kg body weight.

In other embodiments, the subject has a level of MAPK or PP protein or mRNA that is different than a normal control. For example, the subject has a level of MAPK (p38) or PP protein (PP1) or mRNA that is at least about 5%, 10%, 15%, 20%, 25%, 30%, 40%, 45%, 50%, 55%, 60%, 65%, 70%, 75%, 80%, 90%, 95%, 99%, 100%, 5-50%, 50-75%, 75-100%, 1-fold, 2-fold, 3-fold, 4-fold, or 5-fold higher compared to a normal control. In other examples, the subject has a level of MAPK (p38) or PP (PP1) activity that is at least about 5%, 10%, 15%, 20%, 25%, 30%, 40%, 45%, 50%, 55%, 60%, 65%, 70%, 75%, 80%, 90%, 95%, 99%, 100%, 5-50%, 50-75%, 75-100%, 1-fold, 2-fold, 3-fold, 4-fold, or 5-fold higher compared to a normal control.

In examples, the level (e.g., of MAPK or PP) is in a test sample obtained from the subject. For example, the test sample includes blood, serum, plasma, saliva, tears, vitreous, cerebrospinal fluid, sweat, cerebrospinal fluid, or urine.

In certain aspects, methods are provided for treating vascular Ehlers-Danlos Syndrome in a subject in need thereof comprising: (a) assessing the subject for a level of MAPK or PP protein or mRNA that is different than a normal control (e.g. assessing a subject sample) and/or (b) identifying the subject as having a level of MAPK or PP protein or mRNA that is different than a normal control (e.g. healthy subject); and (c) administering to the subject (e.g. the identified subject) a therapeutically effective amount of an agent as disclosed herein including an agent that can increase the activity or expression of a mitogen-activated protein kinase (MAPK), a protein phosphatase (PP), or a combination thereof. Suitably, the subject is a human subject.

Exemplary preferred agents for use in the present treatment methods and pharmaceutical compositions include for example one or more of anismyin, a substituted anismyin, sorbitol, a microcystin compound, cobimetinib, trametinib, ruboxistaurin, enzastaurin, sotrastaurin, or any combination thereof.

In aspects, provided herein is a method of treating or preventing a vasculopathy in a subject, the method including administering an effective amount of an agent (e.g., an agent that increases the activity of MAP2K6 and hence p38 mitogen-activated protein kinase (MAPK) pathway signaling, thereby treating the vasculopathy.

In embodiments, the vasculopathy is vascular Ehlers-Danlos Syndrome (vEDS).

In other embodiments, the agent that increase MAP pathway signaling (p38) or PP1 includes an antibody or fragment thereof, a polypeptide, a small molecule, a nucleic acid molecule, or any combination thereof.

In aspects, provided herein is a pharmaceutical composition for the treatment of a vasculopathy, the composition including an effective amount an agent, wherein the agent increases the activity or expression of mitogen-activated protein kinase (MAPK), a protein phosphatase (PP), or a combination thereof, and thereby treating the vasculopathy.

In embodiments, the agent of the pharmaceutical composition is an antibody or fragment thereof, a polypeptide, a small molecule, a nucleic acid molecule, or any combination thereof.

In aspects, provided herein is a kit including a pharmaceutical composition of any of the agents described herein, and written instructions for treating the vasculopathy.

Other aspects of the invention are disclosed infra.

### DESCRIPTION OF THE DRAWINGS

The patent or application file contains at least one drawing executed in color.

Copies of this patent or patent application publication with color drawing(s) will be provided by the Office upon request and payment of the necessary fee.

FIG. 1 shows a graph indicating that 129Sve background protects Col3a1^{G938D/+}mice from premature death. Kaplan Meier Survival curve comparing Col3a1^{+/+}(n=71) to Col3a1^{G938D/+} mice (n=62), which die from vascular rupture or dissection on a Bl6 background and Col3a1^{+/+}(n=79) to Col3a1/^{G938D/+} mice (n=80) on a 129 background. Significant differences were calculated using Log-Rank (Mantel-Cox) analysis(****p<0.0001).

FIGS. 2A-2C are graphs showing that there are no differences in blood pressure or weight between backgrounds. FIG. 2A is a graph of blood pressure measurements. Error bars show mean±s.e. Asterisks signify significant differences between treatment groups (p=0.1563, DF=1, F=2.091). FIG. 2B is a graph of weights of a cohort of male vEDS mice at 60 days of age. Error bars show mean±s.e. Asterisks signify significant differences using two-way ANOVA (**p<0.01; DF=1, F=12.82). FIG. 2C is a graph of weights of a cohort of female vEDS mice at 60 days of age. Error bars show mean±s.e. Asterisks signify significant differences using two-way ANOVA (***p<0.001; DF=1, F=20.62).

FIG. 3 is a graph showing that 129Sve background protects Col3a1G^{209S/+} mice from premature death. Kaplan-Meier Survival curve comparing Col3a1^{+/+}(n=60) to col3a1^{G209S/+} mice (n=84), which die from vascular rupture or dissection on a B16 background and Col3a1^{+/+}(n=63) to Col3a1G^{209S/+} mice (n=66) on a 129 background. Significant differences were calculated using Log-Rank (Mantel-Cox) analysis (****p<0.0001).

FIG. 4A are images of histological staining (H&E=Hematoxylin & Eosin, VVG=Verhoeff Van Gieson, Masson's Trichrome, and PSR=Picrosirius Red) of wild type and vEDS aortic cross sections. Scale bar=50 μm. FIG. 4B is a graph showing the quantification of aortic wall thickness in aortic cross sections (genotype: DF=1, F=22.20, background: DF=1, F=5.998). FIG. 4C is a graph showing the quantification of elastin breaks in VVG-stained aortic cross sections (genotype: DF=1, F=33.49, background: DF=1, F=0.7051). FIG. 4D is a graph showing quantification of collagen content in aortic cross sections, as measured by normalized PSR intensity (genotype: DF=1, F=17.10, background: DF=1, F=0.4674).

FIG. 5A are images of a representative western blot analysis of pPKCβ and pERK comparing Col3a1^{+/+}to Col3a1^{G938D/+} proximal descending aortas in B16 and 129 backgrounds. FIG. 5B is a graph showing the quantification of pERK levels normalized to β-actin loading control comparing B16 Col3a1^{+/+}(n=4), B16 Col3a1^{G938D/+} (n=5), 129 Col3a1^{+/+}(n=4) and 129 Col3a1^{G938D/+} (n=5) aortas (****p<0.0001, DF=14). FIG. 5C is a graph showing the quantification of pPKCβ levels normalized to β-actin loading control comparing B16 Col3a1^{+/+}(n=4), B16 Col3a1^{G938D/+} (n=5), 129 Col3a1^{+/+}(n=4) and 129 Col3a1^{G938D+} (n=5) aortas (****p<0.0001, DF=14). In all, error bars show mean±s.e. and asterisks signify significant differences using two-way ANOVA with Tukey's multiple comparisons post-hoc test.

FIG. 6 are images of immunohistochemistry of human vEDS patient samples showing increased pPKCβ (red) and pERK1/2 (green) in two vEDS patient samples but not in a control sample.

FIG. 7 is a graph showing that F1 intercross protects Col3a1^{G938D/+} mice from premature death. Kaplan-Meier Survival curve comparing F1 Col3a1^{G938D/+} mice (n=38) to B16 Col3a1^{G938D/+} mice (n=62) and 129 Col3a1^{G938D/+} mice (n=80). Significant differences were calculated using Log-Rank (Mantel-Cox) analysis (****p<0.0001).

FIG. 8 is an image showing the interbreeding strategy for identification of the modifier locus.

FIG. 9 is a graph showing a survival and stratification strategy of mixed modifier experiment. Mice that died from vascular rupture before 8 weeks of age are cases or those considered B16-like, in yellow. Mice that survive longer than 6 months of age are controls or those considered 129-like, in red. Mice that did not die from vascular rupture (documented malocclusion, “other”) or died between 8 weeks and 6 months of age (“Htx-late”), were not included in analysis. Htx=hemothorax.

FIG. 10 is a graph showing that a single locus on distal Chromosome 11 associates with protection. Manhattan plot of mixed background vEDS mice n=91 controls, n=96 cases.

FIG. 11 is a bar graph showing the percent survival of pure background or mixed background mice, stratified by allele at the most significant SNP (JAX00321228).

FIG. 12 is an image showing genes found at the 10.1 Mb chromosome 11 locus.

FIG. 13 is a graph showing that Map2k6 expression is higher in 129Sve mice. qPCR analysis of Map2k6 normalized to Hprt in the proximal descending aorta comparing Col3a1^{+/+}to Col3a1^{G938D/+} for both B16 and 129 backgrounds. Error bars show mean±s.e. Significant differences were determined using two-way ANOVA (**p<0.01, DF=1, F=8.579).

FIG. 14 is a bar graph showing a Kaplan-Meier Survival curve for comparing 129 background Map2k6+I+Col3a1G938D1+ mice (n=30) (higher plot on y axis) to Map2k6^{-l-}Col3a1^{G938D/+} mice (n=18) (lower plot on y axis). Significant differences were calculated using Log-Rank (Mantel-Cox) analysis (**p<0.01).

FIGS. 15A and 15B show variation in Map2k6 results in differential activation of p38-PP1/2A axis. FIG. 15A is a representative western blot analysis of p-p38 comparing Col3a1^{+/+}to Col3a1G^{938D/+} proximal descending aortas in B16 and 129 backgrounds. FIG. 15B is a graph showing quantification of p-p38 levels normalized to β-actin loading control comparing B16 Col3a1^{+/+}(n=8), B16 Col3a1G^{938D/+} (n=9), 129 Col3a1^{+/+}(n=8) and 129 Col3a1^{G938D/+} (n=9) aortas. Error bars show mean±s.e. Asterisks signify significant differences using two-way ANOVA (**p<0.01, DF=1, F=10.37).

FIG. 16A is a graph showing the average dephosphorylation activity comparing B16 Col3a1^{+/+}(n=6), B16 Col3a1^{G938D/+} (n=7), 129 Col3a1^{+/+}(n=8) and 129 Col3a1^{G938D/+} (n=8) aortas. Error bars show mean±s.e. Asterisks signify significant differences using two-way ANOVA with Sidak's multiple comparisons post-hoc test (**p<0.01, DF=25).

FIG. 16B is a graph showing the total number of molecules dephosphorylated after two-hours comparing B16 Col3a1^{+/+}(n=6), B16 Col3a1^{G938D/+} (n=7), 129 Col3a1^{+/+}(n=8) and 129 Col3a1^{G938D/+} (n=8) aortas. Error bars show mean±s.e. Asterisks signify significant differences using two-way ANOVA with Sidak's multiple comparisons post-hoc test (***p<0.001, DF=25).

FIG. 17 is a graph showing that PP1 activity differs between BL6 and 129 mice. Average PP1 dephosphorylation activity comparing B16 Col3a1^{+/+}(n=6), B16 Col3a1^{G938D/+} (n=7), 129 Col3a1^{+/+}(n=8) and 129 Col3a1^{G938D/+} (n=8) aortas. Error bars show mean±s.e. Asterisks signify significant differences using two-way ANOVA with Sidak's multiple comparisons post-hoc test (*p<0.05, DF=25).

FIG. 18 is a graph showing that p38 inhibition increases vascular rupture risk in BL6 vEDS mice. Kaplan-Meier survival curve comparing placebo-treated B16 Col3a1^{G938D/+} (n=17) to B16 Col3a1^{G938D/+} (n=14) mice receiving SB203580 via IP injection starting at weaning and continuing for 40 days. Significant differences were calculated using Log-Rank (Mantel-Cox) analysis (**p<0.01).

FIG. 19A depicts a representative western blot analysis of pPKCβ and pERK comparing placebo treated to p38-inhibitor treated Col3a1^{G938D/+} proximal descending aortas in B16 and 129 backgrounds. FIG. 19B is a bar graph showing quantification of pPKCβ levels normalized to β-actin loading control comparing placebo treated Col3a1^{G938D/+} (n=4) to SB203580 treated Col3a1^{G938D/+} (n=7) aortas. Error bars show mean±s.e. Asterisks signify significant differences using unpaired two-tailed t test (**p<0.01, DF=9). FIG. 19C is a bar graph showing quantification of pERK1/2 levels normalized to β-actin loading control comparing placebo treated Col3a1^{G938S/+} (n=4) to SB203580 treated Col3a1^{G938D/+} (n=7) aortas. Error bars show mean±s.e. Asterisks signify significant differences using two-tailed Mann-Whitney test (**p<0.01).

FIG. 20 is a bar graph showing a Kaplan-Meier survival curve comparing BL6 Col3a1^{G938D/+} (n=14) mice receiving SB203580 via IP injection starting at weaning and continuing for 30 days to BL6 Col3a1^{G938D/+} (n=11) mice receiving SB203580 via IP injection and cobimetinib via drinking water starting at weaning and continuing for 30 days and BL6 Col3a1^{G938D/+} (n=8) mice receiving SB203580 via IP injection and ruboxistaurin via chow starting at weaning and continuing for 30 days. Significant differences were calculated using Log-Rank (Mantel-Cox) analysis (*p<0.05, **p<0.01).

### DETAILED DESCRIPTION

Provided herein are, inter alia, methods, compositions and kits for treating and preventing vascular Ehlers-Danlos syndrome, and related syndromes.

Vascular Ehlers Danlos syndrome (vEDS) is an inherited connective tissue disorder caused by heterozygous mutations in the COL3A1 gene(1-6). The major cause of mortality is spontaneous vascular rupture or organ rupture(1,2). The PLC/IP3/PKC/ERK axis (phospholipase C/inositol 1,4,5-triphosphate/protein kinase C/extracellular signal-regulated kinase) has emerged as a candidate mediator of disease pathology and a key therapeutic target in vEDS. The link between COL3A1 mutations and initiation of the intracellular signaling cascade remains incompletely understood. However, there is a clear signature for increased PKC and ERK signaling in severe (Col3a1/G938134) and mild (Col3a1^{G209S/+})mouse models of vEDS. The difference in severity observed in these models is attributed to the different substitutions for glycine residues that disrupt collagen triple helix folding at separate locations. However pharmacological interventions that inhibit PKC/ERK activation in either vEDS model rescue aortic disease rupture risk, including MEK inhibitors trametinib and cobimetinib, a PKC inhibitor ruboxistaurin, or an IP3 inhibitor hydralazine.

Genotype-phenotype correlations in vEDS have been extensively studied in humans. These data suggest that both the nature and location of COL3A1 mutations can influence disease severity. Overall, substitutions of glycine residues and splice-site mutations that lead to in-frame exon skipping are associated with a more severe phenotype when compared to any mutational mechanism that leads to functional haploinsufficiency(3). Intuitively, since type III collagen monomers interact to form a triple helical structure; 7/8 of the total type III collagen triple helices will be abnormal if the allele produces a mutant protein that is competent for interaction. In contrast, while haploinsufficiency is expected to lead to half normal levels of type III collagen, all of the resultant protein aggregates will be qualitatively normal. While these types of functional arguments allow broad generalizations regarding classes of mutations and their resultant phenotypes, there is a broad range of severity within a given mutational class. Furthermore, the often marked intrafamilial clinical variability observed in vEDS remains impossible to reconcile based on isolated consideration of the primary disease allele, suggesting sources of genetic and/or environmental modification that remain to be defined (6-9).

Genetic modification of disease severity has recently been recognized in other connective tissue disorders. Previously, it was recognized that genetic background had a major modifying effect on disease severity in mouse models of Marfan Syndrome (MFS) (10-12). When the Fbn1 mutation in MFS was placed on an ultrapure 12956/SvEvTac (129) background, aortic root aneurysms and biochemical abnormalities (e.g. high SMAD2/3 and ERK1/2 activation) were greatly accentuated and accelerated compared to those observed previously in mixed background or C57BL6/J (BL6) MFS mice (10).

Provided herein, vEDS mutations (Col3a1^{G209S/+} and Col3a1^{G938D/+}) were introduced onto pure 129 and BL6 backgrounds to assess for modulation of phenotypic severity. Equally dramatic effects of background on phenotype was observed, but the directionality was the opposite to that observed in Marfan Syndrome (MFS).

Moreover, a single significant protective locus was identified, at which a single gene and variant was identified that modified the severity of disease in vEDS mice in a PKC and ERK-dependent manner. These results defined a pathway of natural potent protective disease modification and pharmacologic interventions that mimic nature's successful modification strategy will afford substantial protection to patients with vEDS.

Significant advantages of the compositions and methods described herein include, for example, the demonstration that enhanced MAP2K6 and hence p38 activity is a natural, potent and tolerated mechanism to prevent the sequelae of a deficiency of type III collagen deficiency including vascular rupture, as observed in vEDS. This method is preferable to global ERK inhibition, as the latter is known to cause retinal detachment, a catastrophic event to which patients with vEDS are naturally predisposed.

General Definitions

The following definitions are included for the purpose of understanding the present subject matter and for constructing the appended patent claims. The abbreviations used herein have their conventional meanings within the chemical and biological arts.

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, NY 1994); Sambrook et al., MOLECULAR CLONING, A LABORATORY MANUAL, Cold Springs Harbor Press (Cold Springs Harbor, N Y 1989). Any methods, devices and materials similar or equivalent to those described herein can be used in the practice of this invention. The following definitions are provided to facilitate understanding of certain terms used frequently herein and are not meant to limit the scope of the present disclosure.

The term “disease” refers to any deviation from the normal health of a mammal and includes a state when disease symptoms are present, as well as conditions in which a deviation (e.g., vascular Ehlers-Danlos syndrome, and related syndromes) has occurred, but symptoms are not yet manifested.

“Patient” or “subject in need thereof” refers to a living member of the animal kingdom suffering from or who may suffer from the indicated disorder. In embodiments, the subject is a member of a species comprising individuals who may naturally suffer from the disease. In embodiments, the subject is a mammal. Non-limiting examples of mammals include rodents (e.g., mice and rats), primates (e.g., lemurs, bushbabies, monkeys, apes, and humans), rabbits, dogs (e.g., companion dogs, service dogs, or work dogs such as police dogs, military dogs, race dogs, or show dogs), horses (such as race horses and work horses), cats (e.g., domesticated cats), livestock (such as pigs, bovines, donkeys, mules, bison, goats, camels, and sheep), and deer. In embodiments, the subject is a human.

The terms “subject,” “patient,” “individual,” etc. are not intended to be limiting and can be generally interchanged. That is, an individual described as a “patient” does not necessarily have a given disease, but may be merely seeking medical advice.

The transitional term “comprising,” which is synonymous with “including,” “containing,” or “characterized by,” is inclusive or open-ended and does not exclude additional, unrecited elements or method steps. By contrast, the transitional phrase “consisting” of excludes any element, step, or ingredient not specified in the claim. The transitional phrase “consisting essentially of” limits the scope of a claim to the specified materials or steps “and those that do not materially affect the basic and novel characteristic(s)” of the claimed invention.

In the descriptions herein and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

It is understood that where a parameter range is provided, all integers within that range, and tenths thereof, are also provided by the invention. For example, “0.2-5 mg” is a disclosure of 0.2 mg, 0.3 mg, 0.4 mg, 0.5 mg, 0.6 mg etc. up to and including 5.0 mg.

As used in the description herein and throughout the claims that follow, the meaning of “a,” “an,” and “the” includes plural reference unless the context clearly dictates otherwise.

As used herein, “treating” or “treatment” of a condition, disease or disorder or symptoms associated with a condition, disease or disorder refers to an approach for obtaining beneficial or desired results, including clinical results. Beneficial or desired clinical results can include, but are not limited to, alleviation or amelioration of one or more symptoms or conditions, diminishment of extent of condition, disorder or disease, stabilization of the state of condition, disorder or disease, prevention of development of condition, disorder or disease, prevention of spread of condition, disorder or disease, delay or slowing of condition, disorder or disease progression, delay or slowing of condition, disorder or disease onset, amelioration or palliation of the condition, disorder or disease state, and remission, whether partial or total. “Treating” can also mean inhibiting the progression of the condition, disorder or disease, slowing the progression of the condition, disorder or disease temporarily, although in some instances, it involves halting the progression of the condition, disorder or disease permanently.

As used herein, the terms “treat” and “prevent” are not intended to be absolute terms. In various embodiments, treatment can refer to a 10%, 20%, 30%, 40%, 50%, 60%, 70%, 80%, 90%, or 100% reduction in the severity of an established disease, condition, or symptom of the disease or condition. In embodiments, a method for treating a disease is considered to be a treatment if there is a 10% reduction in one or more symptoms of the disease in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition. In embodiments, references to decreasing, reducing, or inhibiting include a change of 10%, 20%, 30%, 40%, 50%, 60%, 70%, 80%, 90% or greater as compared to a control level and such terms can include but do not necessarily include complete elimination. In embodiments, the severity of disease is reduced by at least 10%, as compared, e.g., to the individual before administration or to a control individual not undergoing treatment. In some aspects the severity of disease is reduced by at least 25%, 50%, 75%, 80%, or 90%, or in some cases, no longer detectable using standard diagnostic techniques.

The terms “effective amount,” “effective dose,” etc. refer to the amount of an agent that is sufficient to achieve a desired effect, as described herein. In embodiments, the term “effective” when referring to an amount of cells or a therapeutic compound may refer to a quantity of the cells or the compound that is sufficient to yield an improvement or a desired therapeutic response without undue adverse side effects (such as toxicity, irritation, or allergic response) commensurate with a reasonable benefit/risk ratio when used in the manner of this disclosure. In embodiments, the term “effective” when referring to the generation of a desired cell population may refer to an amount of one or more compounds that is sufficient to result in or promote the production of members of the desired cell population, especially compared to culture conditions that lack the one or more compounds.

As used herein, an “isolated” or “purified” nucleic acid molecule, polynucleotide, polypeptide, or protein, is substantially free of other cellular material, or culture medium when produced by recombinant techniques, or chemical precursors or other chemicals when chemically synthesized. Purified compounds are at least 60% by weight (dry weight) the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis. A purified or isolated polynucleotide (RNA or DNA) is free of the genes or sequences that flank it in its naturally-occurring state. Purified also defines a degree of sterility that is safe for administration to a human subject, e.g., lacking infectious or toxic agents.

Similarly, by “substantially pure” is meant a nucleotide or polypeptide that has been separated from the components that naturally accompany it. Typically, the nucleotides and polypeptides are substantially pure when they are at least 60%, 70%, 80%, 90%, 95%, or even 99%, by weight, free from the proteins and naturally-occurring organic molecules with they are naturally associated.

By “agonist” is meant a chemical that binds to a receptor and activates the receptor to produce a biological response. Whereas an agonist causes an action, an “antagonist” blocks the action of the agonist and an inverse agonist causes an action opposite to that of the agonist. As used herein, the terms “antagonist” and “inhibitor” are used interchangeably to refer to any molecule that counteracts or inhibits, decreases, or suppresses the biological activity of its target molecule. In some embodiments, an agonist is a “superagonist” when it induces or increases the biological activity of its target molecule. In some embodiments, an antagonist is a “superantagonist” when it counteracts or inhibits, decreases, or suppresses the biological activity of its target molecule. Suitable inhibitors, antagonists, agonists include soluble receptors, peptide inhibitors, small molecule inhibitors, ligand fusions, and antibodies.

As used herein, an “antagonist” may refer to an antibody or fragment thereof, peptides, polypeptide or fragments thereof, small molecules, and inhibitory nucleic acids or fragments thereof that interferes with the activity or binding of another, for example, by competing for the one or more binding sites of an agonist, but does not induce an active response.

The term “administering,” as used herein, refers to any mode of transferring, delivering, introducing, or transporting an agent, for example, to a subject in need of treatment for a disease or condition. Such modes include, but are not limited to, oral, topical, intravenous, intraperitoneal, intramuscular, intradermal, intranasal, and subcutaneous administration.

By “co-administer” it is meant that a composition described herein is administered at the same time, just prior to, or just after the administration of additional therapies. The agent or the composition of the disclosure can be administered alone or can be co-administered to the patient. Co-administration is meant to include simultaneous or sequential administration of the compound individually or in combination (more than one compound or agent). The preparations can also be combined, when desired, with other active substances.

As used herein, “sequential administration” includes that the administration of two agents (e.g., the agents or compositions described herein) occurs separately on the same day or do not occur on a same day (e.g., occurs on consecutive days).

As used herein, “concurrent administration” includes overlapping in duration at least in part. For example, when two agents (e.g., any of the agents described herein that has bioactivity) are administered concurrently, their administration occurs within a certain desired time. The agents' administration may begin and end on the same day. The administration of one agent can also precede the administration of a second agent by day(s) as long as both agents are taken on the same day at least once. Similarly, the administration of one agent can extend beyond the administration of a second agent as long as both agents are taken on the same day at least once. The bioactive agents/agents do not have to be taken at the same time each day to include concurrent administration.

As used herein, “intermittent administration” includes the administration of an agent for a period of time (which can be considered a “first period of administration”), followed by a time during which the agent is not taken or is taken at a lower maintenance dose (which can be considered “off-period”) followed by a period during which the agent is administered again (which can be considered a “second period of administration”). Generally, during the second phase of administration, the dosage level of the agent will match that administered during the first period of administration but can be increased or decreased as medically necessary.

As used herein an “alteration” also includes a 2-fold or more change in expression levels or activity of a gene or polypeptide, for example, 5-fold, 10-fold, 20-fold, 30-fold, 40-fold, 50-fold, 100-fold, 500-fold, 1000-fold or more.

As defined herein, the term “inhibition,” “inhibit,” “inhibiting” and the like in reference to a protein-inhibitor the activity or function of the protein (e.g., decreasing the activity or amount of ERK or PKC, or PLC or IP3, decreasing the ability of ERK or PKC, or PLC or IP3 to bind to a receptor, decreasing the ability of a receptor to bind ERK or PKC, or decreasing ERK or PKC signaling upon the binding of ERK or PKC, or PLC or IP3 to a receptor) relative to the activity or function of the protein in the absence of the inhibitor. In embodiments, inhibition refers to reduction of a disease or symptoms of disease (e.g., connective tissue disorder). Similarly an “inhibitor” is a compound or protein that inhibits a target by binding, partially or totally blocking, decreasing, preventing, delaying, inactivating, desensitizing, or down-regulating activity.

As defined herein, the term “activation,” “activate,” “activating” and the like in reference to a protein-activator (e.g., a p38 activator,) interaction means positively affecting (e.g., increasing) the activity or function of the protein (e.g., increasing the activity or amount of p38 or PP1,) relative to the activity or function of the protein in the absence of the activator.

The dosage and frequency (single or multiple doses) administered to a mammal can vary depending upon a variety of factors, for example, whether the mammal suffers from another disease, and its route of administration; size, age, sex, health, body weight, body mass index, and diet of the recipient; nature and extent of symptoms of the disease being treated, kind of concurrent treatment, complications from the disease being treated or other health-related problems. Other therapeutic regimens or agents can be used in conjunction with the methods and agents of this disclosure. Adjustment and manipulation of established dosages (e.g., frequency and duration) are well within the ability of those skilled in the art.

For any agent described herein, the therapeutically effective amount (e.g., effective dose or effective amount) can be initially determined from cell culture assays. Target concentrations will be those concentrations of therapeutic drug(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring agent's effectiveness and adjusting the dosage upwards or downwards, as described above. Adjusting the dose to achieve maximal efficacy in humans based on the methods described above and other methods is well within the capabilities of the ordinarily skilled artisan.

Dosages may be varied depending upon the requirements of the patient and the therapeutic drug being employed. The dose administered to a patient should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the agent. Thereafter, the dosage is increased by small increments until the optimum effect under circumstances is reached. Dosage amounts and intervals can be adjusted individually to provide levels of the administered agent effective for the particular clinical indication being treated. This will provide a therapeutic regimen that is commensurate with the severity of the individual's disease state.

The effective dose of the agent (e.g., pharmacological inhibitor or activator) of the present disclosure for treating vEDS, and/or a related syndrome may be from about 0.001 mg/kg to about 0.01 mg/kg of the agent, from about 0.01 mg/kg to about 0.1 mg/kg of the agent, from about 0.1 mg/kg to about 1.0 mg/kg of the agent, from about 1.0 mg/kg to about 5.0 mg/kg of the agent, from about 5.0 mg/kg to about 10 mg/kg of the agent, from about 10 mg/kg to about 15 mg/kg of the agent, from about 15 mg/kg to about 20 mg/kg of the agent, from about 20 mg/kg to about 25 mg/kg of the agent, from about 25 mg/kg to about 30 mg/kg of the agent, from about 30 mg/kg to about 35 mg/kg of the agent, from about 35 mg/kg to about 40 mg/kg of the agent, from about 40 mg/kg to about 45 mg/kg of the agent, from about 45 mg/kg to about 50 mg/kg of the agent, from about 50 mg/kg to about 55 mg/kg of the agent, from about 55 mg/kg to about 60 mg/kg of the agent, from about 60 mg/kg to about 65 mg/kg of the agent, from about 65 mg/kg to about 70 mg/kg of the agent, from about 70 mg/kg to about 75 mg/kg of the agent, from about 75 mg/kg to about 80 mg/kg of the agent, from about 80 mg/kg to about 85 mg/kg of the agent, from about 85 mg/kg to about 90 mg/kg of the agent, from about 90 mg/kg to about 95 mg/kg of the agent, or from about 95 mg/kg to about 100 mg/kg of the agent.

In some aspects, the present disclosure includes compositions with an effective dose of an agent(s) of the present disclosure in which the agent may be from about 0.1% to about 20% w/v of the composition. A weight percent of a component, unless specifically stated to the contrary, is based on the total weight of the formulation or composition in which the component is included.

For example, the effective dose of an agent disclosed herein may be from about 0.001% -about 0.01%, from about 0.01% -about 0.1%, from about 0.1% -about 1.0%, from about 1.0% -about 2.0%, from about 2.0% -about 3.0%, from about 3.0% -about 4.0%, from about 4.0% -about 5.0%, from about 5.0% -about 6.0%, from about 6.0% -about 7.0%, from about 7.0% -about 8.0%, from about 8.0% -about 9.0%, from about 9.0% -about 10%, from about 10% -about 11%, from about 11% -about 12%, from about 12% -about 13%, from about 13% -about 14%, from about 14% -about 15%, from about 15% -about 16%, from about 16% -about 17%, from about 17% -about 18%, from about 18% -about 19%, or from about 19% -about 20% w/v of the composition.

A “control” sample or value refers to a sample that serves as a reference, usually a known reference, for comparison to a test sample. For example, a test sample can be taken from a test subject, e.g., a subject with vascular Ehlers-Danlos syndrome (or a related syndrome), and compared to samples from known conditions, e.g., a subject (or subjects) that does not have vascular Ehlers-Danlos syndrome (or a related syndrome) (a negative or normal control), or a subject (or subjects) who does have vascular Ehlers-Danlos syndrome (or a related syndrome) (positive control). A control can also represent an average value gathered from a number of tests or results. One of skill in the art will recognize that controls can be designed for assessment of any number of parameters. One of skill in the art will understand which controls are valuable in a given situation and be able to analyze data based on comparisons to control values. Controls are also valuable for determining the significance of data. For example, if values for a given parameter are variable in controls, variation in test samples will not be considered as significant.

The term, “normal amount” with respect to a compound (e.g., a protein or mRNA) refers to a normal amount of the compound in an individual who does not have vascular Ehlers-Danlos syndrome (or a related syndrome) in a healthy or general population. The amount of a compound can be measured in a test sample and compared to the “normal control” level, utilizing techniques such as reference limits, discrimination limits, or risk defining thresholds to define cutoff points and abnormal values (e.g., for vascular Ehlers-Danlos syndrome (or a related syndrome) or a symptom thereof). The normal control level means the level of one or more compounds or combined compounds typically found in a subject known not suffering from vascular Ehlers-Danlos syndrome (or a related syndrome). Such normal control levels and cutoff points may vary based on whether a compound is used alone or in a formula combining with other compounds into an index. Alternatively, the normal control level can be a database of compounds patterns from previously tested subjects who did not develop vascular Ehlers-Danlos syndrome (or a related syndrome)or a particular symptom thereof (e.g., in the event the vascular Ehlers-Danlos syndrome (or a related syndrome) develops or a subject already having vascular Ehlers-Danlos syndrome (or a related syndrome) is tested) over a clinically relevant time horizon.

The level that is determined may be the same as a control level or a cut off level or a threshold level, or may be increased or decreased relative to a control level or a cut off level or a threshold level. In some aspects, the control subject is a matched control of the same species, gender, ethnicity, age group, smoking status, body mass index (BMI), current therapeutic regimen status, medical history, or a combination thereof, but differs from the subject being diagnosed in that the control does not suffer from the disease (or a symptom thereof) in question or is not at risk for the disease.

Relative to a control level, the level that is determined may an increased level. As used herein, the term “increased” with respect to level (e.g., protein or mRNA level) refers to any % increase above a control level. In various embodiments, the increased level may be at least or about a 5% increase, at least or about a 10% increase, at least or about a 15% increase, at least or about a 20% increase, at least or about a 25% increase, at least or about a 30% increase, at least or about a 35% increase, at least or about a 40% increase, at least or about a 45% increase, at least or about a 50% increase, at least or about a 55% increase, at least or about a 60% increase, at least or about a 65% increase, at least or about a 70% increase, at least or about a 75% increase, at least or about a 80% increase, at least or about a 85% increase, at least or about a 90% increase, at least or about a 95% increase, relative to a control level.

Relative to a control level, the level that is determined may a decreased level. As used herein, the term “decreased” with respect to level (e.g., protein or mRNA level) refers to any % decrease below a control level. In various embodiments, the decreased level may be at least or about a 5% decrease, at least or about a 10% decrease, at least or about a 15% decrease, at least or about a 20% decrease, at least or about a 25% decrease, at least or about a 30% decrease, at least or about a 35% decrease, at least or about a 40% decrease, at least or about a 45% decrease, at least or about a 50% decrease, at least or about a 55% decrease, at least or about a 60% decrease, at least or about a 65% decrease, at least or about a 70% decrease, at least or about a 75% decrease, at least or about a 80% decrease, at least or about a 85% decrease, at least or about a 90% decrease, at least or about a 95% decrease, relative to a control level.

The term “sample” as used herein refers to a biological sample obtained for the purpose of evaluation in vitro. In embodiments, the sample may comprise a body fluid. In some embodiments, the body fluid includes, but is not limited to, whole blood, plasma, serum, lymph, breast milk, saliva, mucous, semen, cellular extracts, inflammatory fluids, cerebrospinal fluid, vitreous humor, tears, vitreous, aqueous humor, or urine obtained from the subject. In some aspects, the sample is a composite panel of two or more body fluids. In exemplary aspects, the sample comprises blood or a fraction thereof (e.g., plasma, serum, or a fraction obtained via leukapheresis).

The terms “polypeptide,” “peptide” and “protein” are used interchangeably herein to refer to a polymer of amino acid residues, wherein the polymer may in embodiments be conjugated to a moiety that does not consist of amino acids. The terms also apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed or chemically synthesized as a single moiety.

“Polypeptide fragment” refers to a polypeptide that has an amino-terminal and/or carboxy-terminal deletion, in which the remaining amino acid sequence is usually identical to the corresponding positions in the naturally-occurring sequence. Fragments typically are at least 5, 6, 8 or 10 amino acids long, at least 14 amino acids long, at least 20 amino acids long, at least 50 amino acids long, or at least 70 amino acids long.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. In embodiments, the percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “identical” or percent “identity,” in the context of two or more nucleic acids or polypeptide sequences, refer to two or more sequences or subsequences that are the same or have a specified percentage of amino acid residues or nucleotides that are the same (e.g., 50%, 55%, 60%, 65%, 70%, 75%, 80%, 85%, 90%, 91%, 92%, 93%, 94%, 95%, 96%, 97%, 98%, 99%, or more identity over a specified region, e.g., of an entire polypeptide sequence or an individual domain thereof), when compared and aligned for maximum correspondence over a comparison window, or designated region as measured using a sequence comparison algorithm or by manual alignment and visual inspection. In embodiments, two sequences are 100% identical. In embodiments, two sequences are 100% identical over the entire length of one of the sequences (e.g., the shorter of the two sequences where the sequences have different lengths). In embodiments, identity may refer to the complement of a test sequence. In embodiments, the identity exists over a region that is at least about 10 to about 100, about to about 75, about 30 to about 50 amino acids or nucleotides in length. In embodiments, the identity exists over a region that is at least about 50 amino acids or nucleotides in length, or more preferably over a region that is 100 to 500, 100 to 200, 150 to 200, 175 to 200, 175 to 225, 175 to 250, 200 to 225, 200 to 250 or more amino acids or nucleotides in length.

For sequence comparison, typically one sequence acts as a reference sequence, to which test sequences are compared. In embodiments, when using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window” refers to a segment of any one of the number of contiguous positions (e.g., least about 10 to about 100, about 20 to about 75, about 30 to about 50, 100 to 500, 100 to 200, 150 to 200, 175 to 200, 175 to 225, 175 to 250, 200 to 225, 200 to 250) in which a sequence may be compared to a reference sequence of the same number of contiguous positions after the two sequences are optimally aligned. In embodiments, a comparison window is the entire length of one or both of two aligned sequences. In embodiments, two sequences being compared comprise different lengths, and the comparison window is the entire length of the longer or the shorter of the two sequences. In embodiments relating to two sequences of different lengths, the comparison window includes the entire length of the shorter of the two sequences. In embodiments relating to two sequences of different lengths, the comparison window includes the entire length of the longer of the two sequences.

Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

Non-limiting examples of algorithms that are suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 may be used, with the parameters described herein, to determine percent sequence identity for nucleic acids and proteins. Software for performing BLAST analyses is publicly available through the National Center for Biotechnology Information (NCBI), as is known in the art. An exemplary BLAST algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. In embodiments, the NCBI BLASTN or BLASTP program is used to align sequences. In embodiments, the BLASTN or BLASTP program uses the defaults used by the NCBI. In embodiments, the BLASTN program (for nucleotide sequences) uses as defaults: a word size (W) of 28; an expectation threshold (E) of 10; max matches in a query range set to 0; match/mismatch scores of 1,-2; linear gap costs; the filter for low complexity regions used; and mask for lookup table only used. In embodiments, the BLASTP program (for amino acid sequences) uses as defaults: a word size (W) of 3; an expectation threshold (E) of 10; max matches in a query range set to 0; the BLOSUM62 matrix (see Henikoff & Henikoff, Proc. Natl. Acad. Sci. USA 89:10915 (1992)); gap costs of existence: 11 and extension: 1; and conditional compositional score matrix adjustment.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

“Nucleic acid” refers to nucleotides (e.g., deoxyribonucleotides, ribonucleotides, and 2′-modified nucleotides) and polymers thereof in either single-, double- or multiple-stranded form, or complements thereof. The terms “polynucleotide,” “oligonucleotide,” “oligo” or the like refer, in the usual and customary sense, to a linear sequence of nucleotides. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness.

Nucleic acids, including e.g., nucleic acids with a phosphorothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent, or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, OLIGONUCLEOTIDES AND ANALOGUES: A PRACTICAL APPROACH, Oxford University Press) as well as modifications to the nucleotide bases such as in 5-methyl cytidine or pseudouridine.; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

“Operably linked” refers to a juxtaposition wherein the components so described are in a relationship permitting them to function in their intended manner. A control sequence “operably linked” to a coding sequence is ligated in such a way that expression of the coding sequence is achieved under conditions compatible with the control sequences

As may be used herein, the terms “nucleic acid,” “nucleic acid molecule,” “nucleic acid oligomer,” “oligonucleotide,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides and/or ribonucleotides, and/or analogs, derivatives or modifications thereof. Different polynucleotides may have different three-dimensional structures, and may perform various functions, known or unknown. Non-limiting examples of polynucleotides include genomic DNA, a genome, mitochondrial DNA, a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may comprise natural nucleic acid sequences and variants thereof, artificial nucleic acid sequences, or a combination of such sequences.

The term “amino acid residue,” as used herein, encompasses both naturally-occurring amino acids and non-naturally-occurring amino acids. Examples of non-naturally occurring amino acids include, but are not limited to, D-amino acids (i.e. an amino acid of an opposite chirality to the naturally-occurring form), N-a-methyl amino acids, C-a-methyl amino acids, β-methyl amino acids and D- or L-β-amino acids. Other non-naturally occurring amino acids include, for example, (β-alanine (β-Ala), norleucine (Nle), norvaline (Nva), homoarginine (Har), 4-aminobutyric acid (γ-Abu), 2-aminoisobutyric acid (Aib), 6-aminohexanoic acid (ε-Ahx), ornithine (orn), sarcosine, α-amino isobutyric acid, 3-aminopropionic acid, 2,3-diaminopropionic acid (2,3-diaP), D- or L-phenylglycine, D-(trifluoromethyl)-phenylalanine, and D-p-fluorophenylalanine.

As used herein, “peptide bond” can be a naturally-occurring peptide bond or a non-naturally occurring (i.e. modified) peptide bond. Examples of suitable modified peptide bonds are well known in the art and include, but are not limited to, —CH₂NH—, —CH₂S—, —CH₂CH₂—, —CH═CH— (cis or trans), —COCH₂—, —CH(OH)CH₂—, —CH₂SO—, —CS—NH— and —NH—CO— (i.e. a reversed peptide bond) (see, for example, Spatola, Vega Data Vol. 1, Issue 3, (1983); Spatola, in Chemistry and Biochemistry of Amino Acids Peptides and Proteins, Weinstein, ed., Marcel Dekker, New York, p. 267 (1983); Morley, J. S., Trends Pharm. Sci. pp. 463-468 (1980); Hudson et al., Int. J. Pept. Prot. Res. 14:177-185 (1979); Spatola et al., Life Sci. 38:1243-1249 (1986); Hann, J. Chem. Soc. Perkin Trans. 1307-314 (1982); Almquist et al., J. Med. Chem. 23:1392-1398 (1980); Jennings-White et al., Tetrahedron Lett. 23:2533 (1982); Szelke et al., EP 45665 (1982); Holladay et al., Tetrahedron Lett. 24:4401-4404 (1983); and Hruby, Life Sci. 31:189-199 (1982)).

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

### Vasculopathy

Vasculopathy is a term used to describe a disease affecting blood vessels. It often includes vascular abnormalities caused by degenerative, metabolic and inflammatory conditions, embolic diseases, coagulative disorders, and functional disorders such as posteri or reversible encephalopathy syndrome. The etiology of vasculopathy is generally unknown and the condition is frequently not pathologically proven. Vasculitis, on the other hand, is a more specific term and is defined as inflammation of the wall of a blood vessel.

As used herein, “vasculitis (angiitis or angitis)” refers to inflammation of a blood vessel, e.g., arteritis, phlebitis, or lymphatic vessel, e.g., lymphangitis. Vasculitis can take various forms such as cutaneous vasculitis, urticarial vasculitis, leukocytoclastic vasculitis, livedo vasculitis and nodular vasculitis. Small vessel vasculitis may refer to inflammation of small or medium sized blood or lymphatic vessel, e.g., capillaries, venules, arterioles and arteries.

Vascular Ehlers-Danlos Syndrome (vEDS)

Vascular Ehlers-Danlos Syndrome (vEDS) is an inherited connective tissue disorder caused by heterozygous mutations in the collagen type III alpha 1 chain (COL3A1) gene. The major cause of mortality in vEDS is arterial dissection and/or rupture, but little is known about the pathogenesis of this disease. Effective treatment strategies for this devastating condition do not exist. The current belief is that reduced amounts of collagen III lead directly to the signs and symptoms of vEDS due to an inherent loss of structural integrity of the tissues. However, early pathogenic models of Marfan Syndrome (MFS) also singularly invoked tissue weakness imposed by failed elastogenesis, but subsequent work clearly demonstrated enhanced transforming growth factor beta (TGF-β) signaling in a mouse model deficient in fibrillin-1, the deficient gene product in MFS. Follow-up work went on to show that TGF-β and downstream cellular signaling molecules were major mediators of disease pathology. Further, therapies that attenuate TGF-β signaling and related pathways, such as TGF-β neutralizing antibody (Nab), the angiotensin-II (Ang-II) type 1 receptor blocker (ARB) losartan, or the inhibitor of ERK1/2 activation RDEA119/trametinib, can suppress aortic disease in MFS mice.

However, similar to other heritable vasculopathies such as Marfan syndrome and Loeys-Dietz syndrome, provided herein are signaling abnormalities that are major mediators of disease pathology in vEDS. RNA-seq profiling on the aortas of mice with patient-derived Col3a1 mutations demonstrated elevated PLC/IP3/PKC/ERK signaling compared to wild-type aortas. Immunoblotting of the proximal descending thoracic aorta confirmed elevated PKC and ERK1/2 activation.

### MAPK—p38

p38 mitogen-activated protein kinases are a class of mitogen-activated protein kinases (MAPKs) that are responsive to stress stimuli, such as cytokines, ultraviolet irradiation, heat shock, and osmotic shock, and are involved in cell differentiation, apoptosis and autophagy. Persistent activation of the p38 MAPK pathway in muscle satellite cells (muscle stem cells) due to ageing, impairs muscle regeneration.

p38 MAP Kinase (MAPK), also called RK or CSBP (Cytokinin Specific Binding Protein), is the mammalian orthologue of the yeast Hog1p MAP kinase, which participates in a signaling cascade controlling cellular responses to cytokines and stress.

Four p38 MAP kinases, p38-α (MAPK14), (MAPK11), -γ (MAPK12/ERK6), and -δ (MAPK13/SAPK4), have been identified. Similar to the SAPK/JNK pathway, p38 MAP kinase is activated by a variety of cellular stresses including osmotic shock, inflammatory cytokines, lipopolysaccharides (LPS), Ultraviolet light, and growth factors.

In certain embodiments, p38 comprises the following amino acid sequence (NCBI Accession No: 095433.1 (SEQ ID NO: 1), incorporated herein by reference in its entirety):

| | |
|---|---|
| 1 | MAKWGEGDPR WIVEERADAT NVNNWHWTER DASNWSTDKL KTLFLAVQVQ NEEGKCEVTE |
| 61 | VSKLDGEASI NNRKGKLIFF YEWSVKLNWT GTSKSGVQYK GHVEIPNLSD ENSVDEVEIS |
| 121 | VSLAKDEPDT NLVALMKEEG VKLLREAMGI YISTLKTEFT QGMILPTMNG ESVDPVGQPA |
| 181 | LKTEERKAKP APSKTQARPV GVKIPTCKIT LKETFLTSPE ELYRVFTTQE LVQAFTHAPA |
| 241 | TLEADRGGKF HMVDGNVSGE FTDLVPEKHI VMKWRFKSWP EGHFATITLT FIDKNGETEL |
| 301 | CMEGRGIPAP EEERTROGWQ RYYFEGIKQT FGYGARLF |

In certain embodiments, the p38 comprises the following nucleotide sequence. Four isoforms of p38 are known:

Mapk11 (SEQ ID NO: 2):
cctcttccaggagcttctgggtcgaggttagggcaacatggggagtagatgtcgtcttgtctcctgaaggctccg
tcacttactaagacctagtaagtctactaggaggtgaaccattaacgccaaacgggggcgtcctcctcaccctgg
ccacaaggcgccttcctcagtggtgccctgagttggcgccgtggcgctggttttttaacctagagcctttacttc
cccctccccccaccactaatgttttttgttgttgttctgaaagtattatcaccacgtgctcaagctcacttttga
caatttcccttcatttctgaggaaggggactggcctgggacccggggaagggcagagggggtaggtaatatgcag
aagatgaaggtggaggagtacaagcaagaagtttcatcgtcacccccatgggagggaagcaggctttttcctttg
tcacggtctccgaggggcaggaagctgtgcgtctgtctccgggaaccctcaacctccacccaccccaccccaccc
ccgtttccccgccctccggtctccaggctggggaaatccgccgggcctcggccccgccccgccgtcccgccctgc
tgcgccgccgccccccagcccccaccccgctttgctcagcggtgctgggcgtggggcgcgggccgggtgctgcgc
gcggggatccggggcgctcgctccagctgcttctgtggatatgtcgggtccgcgcgcgggattctaccggcaaga
gctgaacaaaacagtatgggaggtgccgcagcggctgcagggcctacgcccggtgggctccggcgcctacggctc
agtctggtaggggccggcaaggacctggtgggagctgggcgggaaagcgtgtgcgaccctccagcgcgacgtggg
gttcccggcgtggggaggggcctcgccctgcctccgcccccggtctgcacgcttccgatgaatgaatggggggga
ggggacacggaggaaactttcctactagattccactcgctggcaggtagggggggtcttctaatgccctctcccc
cccccccatctccgatccctttcctagtttttccccaaaagactgaatattgcggcgtcacagctggcgggagag
caagtctctggggccgctaaatggaacagtcagccatccgggttatttcgggcaaggtctaagtagctgcagggt
ctaacgttcccctcagactaagtatttgaaaaggggacccccagcggggtgaggagacctttctaatttggaaga
caccaaaattttcctgtggatcatacccgcgcctccagattcctagaagctcagcggcttgagcctaagcggaca
cctactctcttgtctacaccctaccccagtttacccaacaaaggatctcagaatataggtagggcttggagacca
gagattgcagtctcgatcgtgggcagcatccaggcacctgggattggagaacggctgtagatagcccttgttgac
gggctgatcaagggtgtggacactggtatgtgccccacaccgggaatgggggctgagtctcacatcttggatccg
gtgctgtcatccgaagcctcggtactgctactgcaaccctatagaggcaaacagtcctgctttacagggcccaga
gtgagttggtcatgggatctcgccccatgtctgcagcctgtaaacccaccactgagatgggtaggcttgttctgc
cgacactcgctccttattcttgcttctcaggctgtaccccagccctccacagtgagtagagacaggatacaccca
ctccatcacccatacatacccaaggagtcggacagaggaagagatgatgggacatgttgtgtctgggatgttcag
ggactcagcaagtggaggaggataaggttacacgaaaatacattttaaaaaaataatacaataaattagagggaa
ggagagccagggaggacttggagggggcagggaggaaggtgaagcagatactttgcaggggacttggacattgac
tcagatggagttgtctcggggctggaccatctaggagttaccagggaaaagccagatttcccaagggagacctag
gtctgtctccgctgatccgtgaccctgggtttgttcagtcaccctgctgaggcaggccatgggatgccaacttct
tgtaacttgattgtacagaggcagtcaagtaagaggatcctgctgttctagtggccactccccacccacctccac
cccagaagttacttagacattctgcttatcataggaaagccacctttcttctacaactctctagttccgtgagca
tcccaaaccaccatgccccttccccatcttccctcacctggtgggaatgggtggtaccctgtctccattcaccac
tgcaagggaagggtgcatcctgagacgaagagaatcgagggagagaaggaacaggaatcaccaggggagcaggct
gtgtaaaggtactgtagccagggctccttcagtggcttcgcagctgctccgggtcttgaggcagagggatcagaa
cctggtgaaaagccttaggaccaccgctcagtccacgtggcctaggccccgcccacaaagaaagcgtgctctcat
ttgctctttcgaggctctccaggtgaaggatagaagggtaaagaggtgcagaggtggcgggtccagtttctgggg
gagtggaagtgtgtcagagacatcttcacactggggttaaacatttcctactatccatttgctgagttataaatt
gtgtgtgtgtgtgtgtgtgtgtgtatttttttgtttgttttggtttggtttcttgtgagacaatgtctcactagg
ttttccggctggttttgaactcaatctcgattctctcgcctctccctctctccaagagctgggattacaggcatg
aacaccacactcggcttcaattacaaatcaggaagcaataaacacctccggataatcttgcttccgcaatctgat
gtgcggaggcgtgggggtggggcgtccagggctttgtaggtaagcctcccacggcatccgtgaggaaggaggagg
gctgcttgctgtgggagcgcctgtgggtacagtcgtggtattttccatgccgtggccttgttgtgtgtgtgtgtg
gggggggggggggcgctcaggtttgggctagaatcaccggttctctgccagagaacgctttgtccactcaaaggg
gtctcctaagggaggatcaggagggtcggcgtggaggcagcgcggggcgagcaggcggggacaggcttcctaggt
tttagagctagcaacagaggctcactcccgcacccagcgtcgggtggattcaggccaggcgcttgctctccttat
tcgcccaccttgcagctcggcctacgacgcgcggctgcgccagaaggtggctgtaaagaagctgtctcgcccttt
ccaatcgctgatccacgcgaggaggacataccgtgagctgcgcctactcaagcacctgaagcacgagaacgtgag
aggggggaccgaccggtgatgttgggactgaacaagggacgcatcctgcctggggggcgggacctacactatagg
ggcggagcctctggaggtacagtcccatgtgggtggggtccagaccaacgggacgttgcctaaggctaagctgtg
atcctgcctcggttccctgaccaggtcataggacttttggacgtcttcacgccggccacatccatcgaggatttc
agcgaagtgtgagcgtcctgggcaggcgcggtggtagtctggggagcttagtcggagatggggtggtgagactgt
cctaactcctgatcctgcaggtacctcgtgacgaccctgatgggcgccgacctgaataacatcgtcaagtgtcag
gccctgagcgatgagcatgttcaattccttgtctaccagctgctgcgtgggctgaaggtggggcatgtgggctct
ggggtcccctcccccccccgaccgctctgggttgagggcagagcgaggtggcgcctgggctcactcggtctgctc
tcctctgcagtatatccactcggcgggcatcatccaccgggtaggtgcagctgtgggcaagggtgagggtcatcc
actctgcctaggctcatggcctcgccacactcaatgcccggtgcaccctgcaggacctgaagcccagcaatgtag
cggtgaacgaggactgcgagctgagggtaagggcggttgaggggtgcacagacagtggggccacatagagaggta
ggtgggggtcttacacgagatccccgtggtcctccagatcctggactttgggctagcacgccaggctgatgagga
gatgaccggatatgtggccacacggtggtaccgggcgccagagatcatgctaaactggatgcactacaaccagac
aggtgctgtcaacccttaaagcagtggctttgtcctgtaaggatgagcaggctgtgctgacactgtttagggagg
atgacaaagccgtgggggtaggaagaaaggctgaaggggaggagagcatctctggagctctgtgagaggagcaag
ggattggtgggaccagcttttagaaaggcttctaagctgatgggtgtctgtcctcatgcctcttctcaggacagg
gtatagagtaaggcttctactggtctctttgggcctggttatgcctgagggtcactgggctggctcctccccagg
ctcacaggaaggatttggggttggcagactgatagtctccttattctcccccaacagtggacatctggtctgtgg
gctgcatcatggctgaactgctgcaaggaaaggccctctttcctggaaacgactgtatccttggggagcagggtc
cagctggggtccccttctccagggtgggagtggactctggccctttttggacgtctttcctctgccctatgccat
atcacacattacacggggctccctgtcctgagccacagctctacagtgtaccaggggctgggaccggaaacgctg
gccaccaggtgtttcctgagctaggtagacatcgaccagctgaagcgaatcatggaggtggtgggcacgcccagt
cctgaggttctggcaaagatatcctcggagcatgtgagttagtggccatccgtctatctgttggctcttccgacc
aagcaccagtcatgctggggagggagtagtaagcagaaatggcatcgcatgaggaatgtgagcagggagggatgg
agcttgtactaggaacatccaaccccttcagagagaagcaggtgaaccaggaggccatttcaggtagcagagaca
gtgctgacagactcccctgcttatagacactattgaggcagggaagagttgagcttttggcttttgtaggcacca
tcagaagccaaatgaagtcctggagccccccagacttttgttttgttttgtttttgagacagggtttctctgtgt
agccttggctgtcctggaactcactctatagatcaggctggcctcaaactcggagatctgcctgcctttacctcc
tgaatgctgggaataaaggcatgtgtgggaccactgctcggctagggagcgcccaccctgccccgactttgagag
caggagtctggcttgctggggtacctaccaggcctgttggaatgaatgcatctctctaaaggccttctgcccagt
ttgggccaggaaaatagaagttagttagccctccaagctcttcttatgtgtctccaggcccggacatacatccag
tctctgcctcccatgccccagaaggacctcagcagtgtcttccatggagccaaccccctgggtaaggactggtac
tggatctaggctgggcttcagatctagcattggctatggtgctaagcctcactcactcctgcagccatagacctc
cttggaagaatgctggtactagacagcgaccagagggtcagtgcggccgaagccttggcccacgcatacttcagc
cagtaccatgaccctgacgatgagccagaggcagagccctatgatgaaagtgttgaggccaaggagcgcacgctg
gaggagtggaagggtgagcacagggatggggcgtggggcgtgaggatgggaaagagagcagtgtccgtgaacctg
agggttgccttttctcagagcaagattttacctgtttcatgcaggaactccccagaacttccccaaaggtctggg
atggggaggggaagcagggggtggggtgggggaaggaggatggaaacatatctgtttctgagtgagacatgtaag
ggtgtgtgtgtgtgtgtgtgtgtgtgtgtgtgtgtgtgtagcagaggtttggagggaagtgggcagacagcataa
tctcacccttcccccctttttcattcccctccccagagcttacttaccaagaagtccttagcttcaagcccctgg
aaccctcacagctccctggcacccatgaaattgagcagtgaggcattgcctgaagtgggagacctgagcctgtcc
cctctactcagctttccggctttcctcaagaggcgcctctcaggcagccctgacacttaacctgggaccgacccc
cttgccttgagaaactctacacacacacacacacacacacacacacacacacacacacagaggaatgcacggatg
tgtacatatgcctccctcccattatgtgctggagtctgggcagtagtgtttctggacctaccttggtccacccag
gttctccttgggacctccgtgtgtgggatctgagtgtgtccctgtccttgtccaggtctcttctgctcttgagga
ctatctggattgggaggaaggctaatgggccatggtccctggagactctgagggagggggagtcacagtggttgg
agatgctcaaccaggaagtgagtggctgaaagcagaggcaagcccagtgtccctcctaactgagtggcgtttcct
ctggggatgggaaagctgagaccaccatcagattatctatctagtcagaaaagcctgctgaagatttgtggttgc
ctgtggtatgtgggcacatgggcatgagtatgcaccctgggatacacatcagaacaaatgcaggcatgtgctggt
gtgtatgaatctctgagcacccaagaggcagcagccatttctggtctggtgcctaagttggagataactcttgcc
tcctctccccccaagttcctgttttctaatgggtcttgaatcccagctgtgaactagaccaaaggctctgagagg
tgccaagatccattagtttaagcaaatgtaagggtataggagcagcagcaggatcctgagtcacaagggcacaat
gccagctggagagctgatgaagttgggggtctaatctcaaggatgctaggtggccagcacccacggatgtgagca
tggacattcttgccccgggaccttgattcaaagttgacaaacgtctgtccacgttctacctccaccaacccttga
ggttctgcagctggtctggggtgctatggtttgcaagtcacccctcatctccccatcccccagagaggagctgac
caagtaacttttgaggtagggccctgtttgaaaacgcgttgggggtgtaacgcctccctgaagagacaatgatct
cttgcttatcagggaccaaggggctctggatgtcaagtgcctgcaccaagagtgtgcacaataaaggggcatccc
tcttgctcttgtctgtgcaaaaggctctggatcccattgcttctactgattctgcttcctggaggggtggctgga
tctctgtgcctccgtacacttcctgctagcgccaccgactttgaatttattgtgccccttgatcactcaatgaga
gcagcagtcatttctagtactatcgcagcttcttgggggtgtgtgtgtgaagaacccaggagaccagtgcaggac
aagacacactggttagaaggggcaaaaggtgtaagagccagcacactttggtccctgcacattagggactttgta
cattaggaatggtggacagacttggctccctcttactggtgaggctggggattgtccctagagaaaagatctgtt
cttcctcagggattggacaggatatgggaaatagctttccttcctgactaaggcgaaaaggcaactttagcccag
gatacagctcatgtgtcatgtctatgtcctgaatcttggaccgccagagatctgagtctggcaactggtccagcg
gtggaggttggggccgggggtggggtgggggggtggacctagaaggcaagagccaagtccaaagttgggggattg
ggagcgggagcaaaccca
Mapk12 (SEQ ID NO: 3):
gggtgggtggaacagaggtgtgaaggagtcttagaagggccgataggataggacccatcc
ctctggatcttagactgggaagttgagagccagtggccacaacaatggggctggggaagc
aacctgagacgtttgggagactgaaccaaatccctcctccatatagaaggaggcgaacgg
atcagccccaggggcggggcatccttgggaaagacggggagttgagccagtaaaaggaac
catttgggatggggtatgattggtgtatgggcaggtggaccagacctggggcttgagtag
aacctggtggtctcaagttgcctagaatgggtccgacgttctgggaggacaggaaaggct
ggaattaagagaggcacctgctaggacccaagggccaagaagtctagcgagggtgtaggc
tggccagggtgtttcaacgcctcctctcatctcaggtcgagactgagctatggagcagct
ctcccccacaaccccccctttccaagccttccgcccaaccccggaggcctcctcgggttt
ccgcaggggccgagggcaggcgggggtgtcacgtgctcagggcctgggggccggttggtc
agtgggccgggagggaccactttgcgggccagagacaagggtcgggtgggagtcggcggt
tatagagagttcagggtggcgaccccgagagaagagcccgcaaaggaaaatctcagaggc
gggcaggcgggtagccggcgcggagtacgccctgccacgcagtgacccggggcgcgcggg
cggagcccctgatcccgggtccggtcctggggcgcggtgctccggctggggatgagctcc
ccgccacccgcccgcaagggcttttaccgccaggaggtgaccaaaacggcctgggaggtg
cgcgccgtgtaccaagacctgcagcccgttggctctggtgcctatggtgcagtgtggtga
gcgtttgccgggcggggcggcaggcagggcaggaggcagctggcgattacggggccggtt
cgctagccaagccgtccgccccgcagctctgcagtagacagccgcactggcaacaaggtg
gccatcaagaagttgtaccggcccttccagtcggagctgtttgccaagcgcgcctacaga
gagttgcgcctcctcaaacacatgcgccacgagaacgtgagtctaaagcccgccccagat
gcggatattgtgcgctggcctcttctcatcctgccctcccctgcaaatcctgcccacagt
ctctgcttccaagggcctggtacctcctacctagttagctcccagcttgggtaaactttc
aacaactttcaggaggaaagcagaggcagagcccagatccctttctgggtgacactacaa
tttcagcctcttcttcaattgcttggtttcaccttcggtccacctctaccttcgcagccc
aggcagtggtcaggttatggtctaggcctaaggattcctcaggtctcactccaaaacctg
attggacaatcaggttggtgtgaggagggttttcctggaaggggtacttccttaccctac
cctagtctccctttgcccaccacccgagtcttctattccagacatacaggatcctaggca
ttcagggcaaggcaggatactcgaggtgtgcagccttggctctatcatcccagagccttg
ggtctctgtggataaagggatttacggagcagagcatcgccattagtgttgtgggtgtgt
atttcttcacacccatacagaaggcatctcctttcccgtcctacctgcatatggctactc
ctaagcctcttgcatcaccattgctagccatcagagcattcctcccaaccagggacatta
ttgttattgttgttgttattattattattattattattattattattagatatggttctt
cctgaacctgtgagttctgtccctcccacttctgttctgtacagctgccctagtcctgat
gtttcaatcctcctggatcatcagagtcaatagctccaaggaaaaatgaacatctcgtgg
ctaacagcaaactgagccccgttgcttcctcattgctactcaactgatttggtggatgga
taccagccctgctaggtctgcttctttgcttcccagtaaagctggaagcagtaaacaaac
aaacaaacaaactcagtaagcctggcagtggcagcacatgtacctgggggggcagaggca
gatggtccccatgagttcgagtccagcctgatctaaagagttagttctaggacagccagc
ttctacacagagaaacccaaacccaaagccaaacaaaccaaaccaaacagtaaaaggctg
tgttttcttgggtgtaaccacccctatcctcagagcccccaacttccacctacttactta
tcctgaacccttcttgaacggagagctgtggagctcttcgttcactccccacttctttgc
cagtctgtgggtcacccctgccttgtgaagagcctttacatcaagctactgacctgtcct
cacctggctgtggtggctttcaggatgcctcaacacatttcggggaggctgccattgtaa
cctacccttccatgtggaccctgaggtttagataggctgtatgtctcactcacaccagag
atggggttgccacagggccagccctacccactgctgccgccgccgcctcctcctcctccc
ccccactcttcttcttcttcttcttccttctcctcttcctccttttcctcaagctctagt
ccttggctctgggtcacattctataccacaaaagtgcagaattcataatcctttgaagga
cctgccttgacctccggcacactattccttatctattctgcgctcagctcatgggtcaca
tgactaaagcagtcgcccatctgccctttacctcctgtctattcttgccgcattctgtca
cagaaactaggagggcccatccagcatcatatccttgtttaaacctttccacggctgctc
gctcaatggagcaaagccaaagctgcccctgggatgggagtgtgaggccccccttgggct
tgctctgtcctttccttgattccttcttggcctgggcttcggccacttcctttctgtacc
ttactctgtagggactttcactcctgtgatggccatcaggcacctgacctctcatttgct
catttactcatccgtcctgggatttggatatatgcggccctcctcaggacaaactccctt
gaccccggaggtcccgtcccctaaccaagtactgggaagccaatgtcacattgtctatct
gttgtccaactccctcaatcaaaagctgtgtttcaattggtgcttggtctagacagccag
aggggatggtcccaggaatgacctactgggaattcattcctcccaggggacggtccccac
tttggtcttgggtctgcttcattgggaagaaaaaaagtgtgtgtgtgtggtgggggggat
ggggagacaggggagacagaagactaaggggaccggattgctcagaggcctggagccaac
ccagacctgagactgcccctccccccaccccctgagaatcccggctgtcagcctccaaaa
atagcctgtgtctgtgggcaaatgtaccaggatgggggctgagccgacagtggagacagt
agaggagacagggatagttatgggagggaggattttcagcctggagtacagtgccatccc
gggggtgcactgaggccctgcagctcccgtgtgctctctatcccgtctgctacttgttct
tcaggtcattgggctactggatgtgttcacacctgatgagtctctggacgacttcacaga
cttgtaagtgctagtggctgagagggtggcaggaacctgggtggcttctcggtgtcagct
taagatcctgggtcaggttgaggaccttctccccatagcccagcggttctcaaccttccc
aatgctgtgaccctttaatacagtttctcatgtcctggtggcccccccagccagaagatt
gtttagttgctacttcataatggtacttttttttttgggggggggggtccgagacagggt
ttctctgtgtagccctggctgtcctggaactcactctgtagaccaggctggcctcgaact
cagaaatccgcctgcctctgcctcccaagtgctgggattaaaggcgtgcgccaccactgc
cctgcataactgtacttttgctactgatagaaattgtattagaaatgatatgtaggatat
ctgataagggacaagcccccctccccccagggagtcatagcccatgtaaggaatagggtg
cacactgcccacccactcctcttaccacagtctataccccactcctatatgctcctggga
gccatgaacttgtgtgagtgataataccatttttccttgtgtatccctccatctatctta
acatccctctgacctagatgccttgccgctccaacccctgccccatattcctggttaccc
tatgtgtggcaggaggggcctaggcagctcctgggtaccagcttcaggttctaagttggc
ttgagttcttcttcccatggcccaagtgaggactgagatgtatacctgctcattcacatc
tcatcataacccatgtactattggtaacccctggatactgggatgccctagtaaagccag
actgtctatccttctaagcctggggagttttgggtcctcacagtgcctgaggtattggta
taactaagcaatctaaggtgccaatatcaggtgcagcctcagccgctcaccctgcatgtt
gggaattttaaaaccctgaggtggcctgtggtggcacacacacttgcaatcccagcattt
gggagatagagctaggagggacaggattccaaggctattttggcctgcctgagaccctct
ctccaaaaacaaaagcaagggctggagagatggttcagtggttaagagccctgactgctc
ttccaagagcacgtgggtttgattcccagcgcctatctggtagctcacagcttctgcaac
tctagtcccagaagagttgatatcatcttttggtctctgtgagcactgtacatggcgtgg
tccacggatacacgtggaggcaaagcatccaaaaaataaaataaagacaagacaaggctg
atggtagtggtgcaagcctttaattccagcacttagatgacagaggtagaggcagatagc
tctctttgaatttgaggccatcctggtctacacggtgagtttcaggccagacaggactgc
agagtgagacctttcctcaaaaacaaacagagagccctcaaacccaaacaaaatacccct
gacacacagcccactccttgcttgccgttctggaaagaggggatgtgggcatgggttgag
aggtgtaattgtcaaggacagtgtgcccagagggtgggttgtggtctagcacggcatggt
aggggtagaaacatcataggatggtatgacccccccccccccattgctagtgatgaatgg
acactctagggtaatgatttggtgggcttctgcagctacctggtgatgccattcatgggc
actgatctgggcaaactcatgaagcatgagaccctgagtgaagacagaatccagtttctt
gtgtatcagatgttgaaggggctgaaggtaggctttgcagaggtggccatagggaggcgg
acagagcttcccgtttgggcagggtaagggctctgtggcttggagtatccctgagctgac
tgctgctgaactctttctttcccccagtatatccatgcggctggtgtcatccacagagtg
agtcctggcggggctgggagattgcagctgggctcctctccagctgtgtagacagatgtg
ctcaaccccagtgcccatggcttaactatgaagggcaagataggtctgtggtggggatgc
tttccctggggaccaggaaagcccctcttgctcttccccagagggaccaggccagtttta
ggtggccctaataatgtgcagcctgagccctgctctggcaggtgggtcatagtggccacc
tttcttctctcaggacttgaagcctggcaacctggctgtgaatgaggactgtgagctgaa
ggtatcgctgggaggggaagatgaccacggaaggggttgggggaccccttctacttcccc
tatgggtctgcttgacccttggccgaggcaaagggacagaactcagacatttgtgcagag
gctgagcagctattaggcaaagagtggctgaatatccctagtcactcagggctcctaaag
gtgattaggggacggtgaaacagaagaaaacctttggcatttgacctgaggtgggtaggg
ccaggtgtactgaccacagggagaatcctgggggcgcaagaccaaagggtggcaaacagt
ttagcgtagaaactcaagcccagctctcttaaggtcttctggaccatatctggtctatcc
tcagtgtctaagttccacctcctcgatacttctttagatcctagactttggccttgccag
gcaggcagacagtgagatgacaggatatgtggtaacccggtggtatcgggcaccagaggt
catcttgaattggatgcgctacacgcagacaggtaagaggctatccagagactccagtgc
acaccatccacatttacacattcattgtattttaaaattatttttgtagtatcggggatg
gaacccagagtcttattcatgctagacaaatgtcctgtcactgagttacatctcagtccg
tctgtctgtctgtctgtctgtctgtctgtctttctttctttctttctttctttctttctt
tctttctttctttctttctttctttctttctttcgttctttcttccttccttccttcctt
ccttccttccttccttcctttctcttccttcttccttccttccttccttccttccttcct
tccttcctccctccctccctccctccctccctccctccctctctctctctctctctctct
ttctttctttctttctttctttctctccttccttccttccttctttttgagaaagaatat
atatgtgtatgtatgtatatgtatatttggtatggtttggttgctttgaagacagggtct
tactatatagctctggctatttctggaactctctgtaggccaggctggcctcgaactcac
agagatccacttgcctctgcctccttccttccttccttccttccttccttccttccttcc
ttccttccttcccccctctctttctttttacctaccttccttccttccttccttccttcc
ttccttcctttctttcttttttcctgtatagatcctttatagtcctgactggcctggaat
tcaatgggcagaccagactggccttgaatccatagagatctacctgcttctgtctgttaa
gagctgggaattaaaggctgtattaccatgtctggccccttcttaattttttttttttcc
gagacagggtttctctgtgtatccctggctgtcctggaactcactctgtagaccaggctg
gcctcgaactccgaaatctgcctgcctctgcctcccaagtgctgggatcaaaggcgtgcg
ccaccacagcccggctcttaatttttataagaacttattttatgtatatataagtacgta
tctgcatgtatctatacatgcagatacatgtatccatacaccacctgtgtactcggtgcc
tgcagaggccagaaggcggcattggattctctaggagtggagtcacagatggctgtgagc
tgtcatgcagatgctggaaaccaaaccaaggtttctggaatagcatcccatgatgttaac
tgattagccacctctagctcccttcatctctggatagggagccatttataggcactacct
ctgggaggtctgccacccttctcaatgacagcctctgctctgtgcctatgtgcacgtctc
cccttcatgtatacttgtctcccacgtgtggtctctttgtgcatcccccttttcttggcc
ttcacggatatgaatgccacccttgcatgtggggcactgatgggacttctcagtggacat
ttggtccgttggctgcatcatggcggagatgattactgggaagatcctgttcaaaggcaa
tgaccgtatccttctatgtagagcacccgttgacttcggccaagatgtgggtgctgggtg
ggcccccctctgtccctgcattgtattcctgaccttggtacagacctggaccagctgaag
gagatcatgaagatcacagggacgccccctcctgagtttgttcagaagctacagagtgca
gaggtcagtgggagtgggtggtgggctggacttgggcttgggggctggcctatcaccctt
tttccctcacaggccaagaactacatggaaggcctccctgagttagaaaagaaggatttt
gcctctgtcctgaccaacgcaagccctcagggtaccgctggatggggtgaaggtctcagg
gtactgggtgagtatgtcccctcctgtggcttggcctgaattgctcttcccacagctgtg
aatctcctggaaaggatgctggtgctggatgcggaacagcgggtgacagcagctgaggcg
ttaacccatccatactttgagtcccttcgggacactgaggatgaacccaaggcccagaaa
tatgacgactcctttgatgatgtagaccgcacccttgaggaatggaagcgtgagtgtggg
ggttccatacaatgtctggctttggaccctgtgtgccctgggtgagtgagcctctgcccc
tggggtgcaggtcagataagagctttgggaggtcaaagggcattgtggggcagtgcaact
caaccaggttgacttctggctaggggggaaggcagtggtaaagagtgtcttttgaaggag
ctccgacccacaagggctggcaggtgatggatgctgagaactgtggagcaggtgtgtgtg
ctttgtgcccttggcttactaactccctggccgttctctccccaacctctatattgccta
gcatcctcatgtccagaccacactggagctctctctaccactgccggtccttatgacttt
cttcttacacctttaaaatgtatccttacctctccttactccatttaaagacctctcctg
aaataactatatcttatgtactgcccatggaagacagtctattcccatgcaggccaaagg
gcccagcttttccctctcatctctagccctagatccctccatagctctgtgccaaacctc
caattctctctttgagaactggcctcagagaactctctcagtttgcctactgtcccctga
gctgggactagcctcccgtgtccttgccagactgacaggtccaacctggggagccaggag
gtttgaggacaggtccattgcgacatgctatcccttgtcttgtctgcctcagaccccagg
cccttgttcttgcaccctggtatgggaaagactggatgggtagaggttcttgactcatgt
cctgaaagtcctggcaggtgttcttcccttctgtgttgtcgtgtgcttatgagtttggtt
ctgtgtgatgaaatccttctgctgctcaacaaaataactcccctaaccacgggccacaag
atccgttctgtggtctccatctacacactgtggctgggtgcttttgctttcaccatagac
cacaggaggcacacaactgagcccttgagcaggctcacacactttcttctcacttccttt
tttttttaatacaagcctctctctcttattattattattttttgaacacaaccccccctt
tttttcttttgaacatacactttcatcccttgttttcctggggcaatctagcttcacagc
agtcttcagtgtttttaagtggattggcccacttaggattagatttaaggcccatggggt
gctggcttccccccttagttgaaagctgtctaactctatcgtttgcttctcttcgcttcc
cgcaggtgtgacttacaaggaagttctcagcttcaagcctcctaggcagctaggagccag
agttccaaaggagacggctctgtgacgacctctgggtggtttggggggtatccaaaggag
gttggctcggagcttcacggcaccttggcttcccttctctggaaaaggaatcctggttaa
caccccgacagtgcctggagcttgtatcccaagtcttccacctggacatgctgtgtagac
ccttgaatcatgaaccctccatctccaaacctgttcttcggctttcgagtgccccagatg
accctggaagaacatctaagctttctgtccaagacccctacccaacatgggactagcctt
tgaattctggagttgtacatgaaatcagtattcgtgaaaaagcttcagagtgagcagagc
ttaggagacaagtgccagacctgagctctgctcgctctggacaatgccaaggccaactcc
tgagacggaatgagacagaggttttggggacactgactcagggacatcatctcttctgga
agtgggtggattctcttacacccttagcctggaattcgaaccagccatttggtgttgcct
aagtggctgggggcaaataaacccttttgtagatctcccattctggttgtgtggccccat
gccttgagcacccatcagttctccaagctgctctgtgcacaaaaaggagctaacctgagc
tctatgtcctccagctaatagaggtgccagtcccctgactcttcccaggccacagtgctg
gtgccacttagtccttgccaatgcttcaagccacattagggcttggcccctcccttcttg
aatgactgtactcttaaggctcagagtctgagcaggaggtctggagactgatgcaaggga
ggatggtggggaagatgatgttaagacccagatagagggatgcctccttctttctgagtt
tgcagcatttgtcttccaatggtccagacttctagtgaggaaggtcttagctctctacca
gcatttccctggcagcctccctgaacccatactgtcatctgcaagccattagacacgaga
ccacacttgggctggttagatggtccatgttcttcctcatgtcccaaggacaggtgcgac
agggcaggacataagggtgaggtctttcattcccttcatttgacaagttctgggcaaata
cactttgcccagtctaatcttgtcagaggagatgccatga
Mapk 13 (SEQ ID NO: 4):
aacctatgctggtctccagtttactgcgtagtcaagaatgacgttatttctctatccttc
cttcctccctcccttcctcccttcttctccccttccctctcttcccaccctctctatctc
tccctcctttcttctatccctctcttccttctctgttcattctctgtatctctcctctct
agtctctcctctcccttcttcctcagggtctcacctagtctggcattgaactatgtactg
aactggactcaaaatcgtagcaattctcctgtctctgcctctatctcctgtgttgagtac
agacctgagccaccacgccttacatgtgaagggtatatttcttaaactagcccaactagc
aacaagttgagcgcccccccccccgcgccctaaccccattcccaactcttcctacagtct
ctaggcagcctcgcaggcacccctttcccgcagggcacccccctcctccctcagcacctg
cagcaggtggctggtggggcctggggcgttcgccggtgatgggcggagtccggagcgcgg
gcgagcggtgggcgggccggggcgggaacgtacctgggcgaggcggcaggtgcgccgccc
cacgggggaggagcgggcgcggggcgggaggggaccgcggcgggacacccgggccgggag
cccgcgggcagccaccgcaacctcctcgcaggaccgccaccacggggtccgggatgagcc
tcactcggaaaaggggcttctacaagcaggacatcaacaagactgcctgggagctaccca
agacctacctggcgcccgcgcacgtcggcagcggggcctatggagcggtgtggtgagacc
cggggaccccaggaatcagaggagccacgggccacccctcggcagcctccccgagtgagc
ccggtggcagtggggcccctgtgggttgagtcgctggcgaatctggttctccaaggctgt
aactttttaaaagggatctcggagttcgtgggacagatccctgcggttgcagatgaggcc
ccaagaggggaacttgccaaatcgcagagcagggactcctggcctgctctgcatcctgtc
agcaagaactgcttctctccttgctctgccgccccttccttggcgtcgccccctactcac
acacactctctctctctctctctctctctctctctctctctctctctctctttctgctgg
tctccggccagcctggggagcacagaatgggggtggcctaggacagggctggagggatct
ccagtttaggccctttgcgccgcccctccccctcacctgcctgtgaccccgcagctcggc
catcgacaagcggacaggggagaaggtggccatcaagaagctgagccggcccttccagtc
ggagatctttgccaagcgcgcttaccgcgagcttctgctcttgaagcacatgcaccatga
gaacgtaggctgcctcggggtgtggatgtgccggggatcggagggcagggagaggggccc
aggaggggcctgcccagggtcgtgcgcgcagggccaggagaaagaacttcctctgctcca
ccctggtttctcagctctctaaggagagcccaatgagaagatgccagggcctgcccagct
cgcttcccagaaaacacacaaccttttcttgattgtgaggaggaagtaggcacaggggag
cttcgcgattggttctgtgaggactccagtccgatgagcctgggcacttgagctaaagct
gcctccattctggccactctaagtctaacaataaatactgagtatttaatgcatgcgggg
gtgggggctggctaacttttataatcctctccgtaacacatgccaaggcacagaaaggtt
tccccggttctgcagcggcagagctgaggttggaaggggcgggaggcagagcctggtgcc
tgaccatttgaccatcctgtctgatggaaacagaccccgtgacccagctctgggccaggc
agaagggctagggtccatttcaaatagagccttgtacatcgtgaaccctaatcctttcag
ataacctttcctcagtggcctccagcttcgaggtggaggggacttgcctctagctctgga
cagactgacagatgtggctagagacccggggtccaaggctagagctgaggtccagagtcc
caaggacctggagttctcatccttctctcctcttccccccttgaccctcaagatttgtta
gttacaagtgggactgttcaccccaaaccccaaatttgggatcaaacttaccttgttctc
agccatctctccagcccaggcaaatgtcctcttctgcctgggcccccaaactgttcccat
gccagctggggccaccttccgtagcagagggtgacctggatggtggccctgggtggctga
gaccctcaggacagggaagaatggcgttggcagcgcaccctcactggtggcctctctcca
ggtcattgggcttctggatgtcttcacccctgcgtcttcccttcggagcttccatgattt
gtgagtcgggctggattggggtgttggcagatcttaaggctggctcgggaatactttggg
gcagggtagcttccgcgcagaaaggggtccgtccccccccacctagcagttcttgtgcct
ctgcttttacattgagatcttccctggagaggtcccttggctgtgggtggaagggctgta
gggtacctgccccccccccccagagctgtctacacagctcctacccttgttatcagaccc
tggcaccaagaagagctgctggccctcacagggtgtgtactgactctcctcacccattcc
tggtggccaggtgtgactttggacttcatgatacctagagggtttttttttcccctagaa
ggaagtgaggatcagatagcccggacaatctggctgatggggtttggctgttacaaagtg
tcaaagtgtccggttttgctggcaggtgcctgggcaaggatgggtgtttggatgttcctg
agaattccaggtgttagtggcagcttctgccaggcttcacatcctagtcaatgctacaga
ggctgcgggagtgttttggtaccttgtgtgtttatgtgtttaaattggacctctcctgta
gcccagtctggcccccaactacctatgtagacaaggattgcccttgaacttctgatcctc
ctgcctctgattctcaagagctcggattccaggcactggtgctgagaatcaatctcgggg
ttttgtgcgtgcttggcaagagctctaccagcctagctatgtccccagcccactaagagt
ggaaggtgttttgttttgtttttttccagacagggtttctctttgtagccctggctgtcc
tggaactcactctgtagaccaggctggcctcaaactcagaaatccgactgcctctgcctc
ccaagagctgggattaaaggcatgtgccaccactgcccaacaagagtggaaatcttaagc
agagtgctgatactgtctgtcagatttggagggcatggccagcagttctgagaaaagagc
aagaagcccctgggaagaaacaagtgctgagagcccccccccacacacacacaccttgat
ctgagttacagtgtggggtccccagggcagacctgaggcccactcaagccaagctgacag
agctatgtcatctgtttggacatggtggctgagggaggttgggacaggtgactaggagtc
ccaatccctgcctggaatataagtaggaagaagagacctcaaggaagagataatgaactt
gaccttgggtccttgactcttccagagcctgtgaaatcacaggaggggggaacatggatg
tagaccaggctagcatcctctccatgggtgatggctaaacgctggggactaaacatggta
agacaagatacaaggatgttggtccagagggcacatctgtgagaaggcaggaagggtgag
caggtgagaactggagagagaggagggctggcaggcacctgggagtcaacagaaggacac
acaggaggagtcactgttcttgacgtggtgactgtgaccttgagcccaacatattaggac
agacacaggtaccatgactaggttatgagaggcagtgtatttaggtggtggagaaagtga
gataaactgtatatattgtatattacacatacacacatgtacacatacatgtacatatac
atgcacacactctcacacatatatacatatacacatatataaatatacatatacatacac
atatatgtatacatatatgtgtgtgtgttggtatgtgcacatgcttgaaggagcccctga
aatcaaagacagttgtgagttgcctgatgggggtgctgggaattgaacttgggttctcta
ggaaaactagcaggtgctcttaaaagctgagtgattcttccagtgtccagcaatagcttt
cagaagcaaagtccacagcaaggtagtataaatatgtaatcccagcgctcaagaggtagg
tggagccctttgggttcgaggccagccttgtgtacatgttccaggccaggcaaggctaca
tgatgaggccctgtctcaaaataaagggaaaaaaatgtgtgcatgcaagacacatgtgat
cgtgtgtgtgtctatgtgtatgtgagagagagagagagagagagagagactgtgtgtgtg
caagtgtgtctgcacgcgcatgtgcatgtgtgagtgcttgtgtgtgtgcacatgtgcatg
cgtgtgtatgtgcatgtctgtgtgcatgtgtgtgagtgcacaacacacaccttttataat
ctattacttttttttttttttttttttttttggtttttcaagacagggtttctctgtata
gccctggctgtcctggaactcacaactcacttggtagaccaggctgggctcgaactcaga
aatccgcctgcctctgcctcccgagtgctgggattaaaggcatgcgccaccacgcccagc
taatctattactttttgtcttttgttttttatttttttattatttttattatttttttac
tttttgtctttaacgttgctcttaccaccttgagtcacacttcccggttgctgaccctga
cctgtttcacttgagaacctaatagctcgggtcccttcttctccaccatctgaagattgt
ttcctgcttccttcctggcagggcatctgttttatagttagcagagccagctggggtggg
tgtgaccctctgcaggagaaacttttggttagaggtaggaatgctagcagatgctgtggg
cttgctgctggtcagctatgcgaggagggcagagccagggtacctttgtatgaacgcagg
cgtgtggtgatacaaagtgtgacctagtctgttgttcaccctgagctcggccagccaggt
tgctgtggccgacattgcaggagcccaggctgggcactgctcatttttcatgtatcctct
ttgtttgttttctgtgttattgtgttcatatgtagggagctgcatatgtgtatgcagcta
tgtgtacattgtatgtgggtactgaggcttcaactcctgtcttcctcatgtcctatttac
atatgtgtgcttatatgtgcatttatctggaggtcagaggtcaacctctgatgttgttat
tccttaagtgctatctaccttgattgtttttcttatgattgtagttgtgtgtatgtgtgc
gcgcacgtgtgtgtgtgtgtgtgtgtgtgtgtgtgtgtgtttgtgtgtgtgtccctgcat
gagttttgtgcaccatatgtatgcagtgcccacagcagccagaagagggcaccagatcct
ctggaactggagttacaggtgagcctcaagatttggatgctcggagccgggcgtggtggc
acacgcctttaatcccagcacttgggaggcagaggcaggcggatttctgagttcgaggcc
agcttggtctacaaagtgagttccaggacagccagagctacacagagaaaccctgtctca
aaaaaccaaaaaaaaaaaaaaaaaaaaaaaaaaaaaaagatttggatgctcggaactgaa
ccagagtcctctgaaagattaaagccatctcttagtccccttccattttttttttttttt
ttgaggcaaggcctctcactgacttgccaactaaactaggcttgctggctaatgagccct
ggggacccttctgtctcctccctaccagcactggggttacagctgaactccaatacaccc
agcttttatacgtgggtgctaagggttgaactccagacctcctcatgcatctgagcactg
tgtcagctgacccagctctccagccccgggactcactatgtagcccaggctgggtttaac
acgttgcaatctctaagtgctgggattacaggcatgaactatgacacctgagtcgtcttc
tgccttcttgcctggtgttgtcttcaaggttactgttgtgcccgacttctgtccccaccc
caccctgacctctctgaggtcacacctgggcctgtgggaggctgggaactgtgctaaaaa
acactcctgccttccagctacctggtgatgcccttcatgcagacagacctacagaagatc
atggggatggaattcagcgaggataaggtccagtacttggtgtaccagatgctcaaaggt
ctaaaggtgggtggagcctacatggtagccaaggcaacttccttgttgagagccagcccg
ctgttgtggggcagaggagggttctcgatgctggtgtggaagctgacgtctctgattcac
atctctctctctctctcattctctctctctctctctctctctctccctccctccctccct
ccctccctccctttcagtacatccactccgctggcatcgtccacagggtgagtgctttct
ctatcatggttccggggaatgagttttggggtaggaacagggtactctggtggccaaggg
attgggagtgacagttctcaggagacagaggctgtgagtgtttcagccgtgagagggctg
atgtagaatgttgagagaatggctgtgttgagaagttggggagatgtttgtgattgtagg
tgtgtgtgtgtgtgcgcgcgtgcgtgctgcccccaaaacccctagctggcacttcaggct
ctaaggacaggaacatggtggaaaaggatgtgtagcccaatgtcattttacagatgagga
aactgaggcctgaaagagaagtgagctggcccagctcctgttgcagtgtagtgaaggatg
tgtgtatgtgtgtacacgcgagtttgtgtatgcactcatgtgcgtgtgtgtgtgcgtgca
tgtacccatgtgtttatgtgtatgcacttgtgtgtgtgtgtgtgcacgtatgacagatct
gacaccacctgctggctccgcctcagtctgatgttccctttccgtcgtcccctcccagga
cctgaaaccgggcaacctggctgtgaatgaagactgtgagctgaaggtgggtcacagcag
gcccaccaaggatggggaggtggatagaattgtgggaggtaggctctgtccctcagaacc
cctttcctcagacagcttgttatccctgtgtcccttgagccatagatcctggactttggg
ctggcccgccacacagacactgagatgacgggctatgtggtgacccgctggtaccgggcc
cccgaggtgatcctcagctggatgcattacaaccagacaggtcagtgcttggctgccctg
gcagccctggtgacctacactggaggtctccagacagttcagacaacacttttctttcct
tccagtcgacatctggtctgttggttgcatcatggcagaaatgctgactggaaagacact
cttcaagggcaaggactgtatccttgaagaattattcattatacgtagttttattttttc
gacagtagtcctggctggcctagaacttgtgatgtagttcaagctcgtctctgcctcctg
aatgctagggttaaaggtgtgcactgccacacctgacttagtgagtgtcattgtgaaacg
gggtctcctgtgtgtgtcccagagagcatctcaagctcatgacgtagccaaagatgacct
tgaactgctccctttcttcctgacacctcccaagtgctagggtcacaaacgtgtcactat
gccggggatagaacccaggatttcatgcttgctaggcaagcactctatcctctggaggcc
cagcccctagaatggttttaaagatttatttttatttatgtgtatgtctgtgtgagtgca
tggcgtgtgtgcaggtgctctgagaggccagaagagggcaccagatcccctggagtggga
gtcacagggttttttatttttgttttttgttttttaagcactggggcatgggaactgaac
ttgggtcctctggaaaagcatcaagcactctccagcccctggagcagtttgaaatgcaga
taatcaaaagataatgagtcctttctgcccccacctctgctagggaaccctcaggagcca
ggcaggggcagcccactcagctcagaatgaagaagaaccaggggaggggaattaggcgtc
gtaggtctaagtagcctccttctgacctgggctgtctgtcgggaagccctccagggacct
gctccaaccgtgccaagagcctgccgtggttaggtggggctggagccaggagtgtctgcc
aggctggctagagccaggctagaggcagggcaggcaagctcagaacaccgcagagagcaa
ggctgtgccttaactggctgccaagacctggaccagctgacccagatcctgaaagtgact
ggggtgccaggtgccgagttcgtgcagaagctgaaagacaaggcggtgagtggctctgac
tggctctgactggcaccttcttatgggctccctctgccctggcagaccctcttttcctca
tcttatcctggactttcgacagggaagtcatactttctcccctaaggccagaacctgaag
acattccatatttttctgtcttccaggccaaatcctatattcagtccctgccccagagcc
ccaagaaggatttcacacagctcttcccacgcgccagtccgcaaggtgagtcctggctgc
gatccccagagtgcttgcagcccttcctagcagccctctgtcccatgccaggtgtaaggc
tcaccaggtgcggggctcactaggtgcaaggctcaccaggtgcaatgctcaccgggtgca
aggctcaccaggtctggggctcaccaggtgcaaggctcaccaggtctggggctcaccagg
tgcaaggctcaccgggtgcaaggctcaccaggtgcaaggctcaccgggtgcaaggctcac
catgtggtctgtccccgcagctgcagacctgctcgacaagatgctggagctggatgtgga
caagcgtctgaccgctgctcaggcactggctcaccccttctttgaacccttccgggaccc
tgaggaggagacagaggcccagcagccttttgatgatgccttagaacatgagaaactcag
cgtggacgaatggaaacgtaagagatgttcccatcttccctctgcctgcccaccctccag
gcctaaggggacacacctgtccctggtagctgtccatctcctgtggggttttctccttga
gctggtttctactctactatctgagggtgaactctcccaggcccatcctaaagcgcagtt
actttccacagaacacatctacaaagagatctcaaacttcagtcccatagcccggaagga
ctcacggcgacgaagtggcatgaagctgcagtgattgatggctggcctccatgtagccca
ggttggcctccatgtagcccaggctggccttgagtcttcctcctctccttttttgagaca
ggatcttactgtgtagccctggagccctggctagacctgcaactcaagagatccacccac
ctctgttacctgggtggtgggattaaaggcatgcaccactgcgcccagctcaatgcccct
tttctgttggttttgagatagcatctctcaaactcaggcacttcagcactaggaagtctt
gtggtctgcagaagctttccagataaagtcacatcctgaggttcagggggttgtgaactg
gggtggggggtggctggggacaataacttgctactgatggtgcaaaggaggccactcctg
gaccccttgttctgttctctgccctgttagggagccaagtatgttagatcccaggggttg
cagcgcagcaggctgttgctggcaggtattttggatgcctgataagcaatggagacagga
gaccagggcgggtcagtgagtgcagcagcagacatgggttcttcctggctaaggttgcag
acctctgttctttccaagttctttccagtgctctgcactgagaagaagcacctcagagtc
agacggcagtggcagcctgactcctccttctgctttggtagaacgagttcagatacccag
gcttgtttgggtttctgccttgggtttctgacatctggtatcatgtgcaccttaagggag
gaacctgatttcaataacacacagtgtatctgaggggccaaagtagaacccttctgctgt
ctgtgttccctcttccctgggtggcctgccatgggactcatgagtccgaggatctactgt
gacactgacccctacattcaaggctgtgacaagtcctgcagaaaagacctctgttcccta
tcgtttattccagcgctttccagtggggcttcactgtcccagaggctcagtgaaggcagt
taagtcgggtgttttgttagaatctgtgccatataaagtagacacgctggtgaatggcag
gaggtgagttacccataatgcaccgttgttaggccatgtcacgtgttcatgcaagacagt
agccgggggctggctgggggcagagggcagtgtcctaggatctgctctactttataaaca
tgaagcacccaggaaataaagcttatgtgacggcaacagagctcctcggcctcagagatc
tgccacagatgccacagtggaggctgcggcaggcaggattggaacccatgacatcagctg
cacctggatgccaggcccacctacacaccatctgtagtactgcctccccaagccctgtca
ctgccagttgtgtctgtactctgtcccccccaacccccatctgagtggccatgttgtcat
atggggaggagactttgtttgggactgactggtggccagctatcaccctgaccttacagt
tagttgcaaggcaggctcaaggttccccgtttcaaacatctcgttttgagcctccctctt
ggtcccagagaagaggcttggtcttctgtctcaacattcacacctcagtcatttttctca
gattttagttgtccctgctttccagattcctggatttccagatgccagggcggccagccg
aaaggcctaccatggacactgtcagctgagaggctctgacaagccctgaagaaaataaag
gttttttttaagctttatttattattatatctaagtacactgtagctgtcatcagacaca
ccagaagagggcatcagatctcattacggatggctatgagccaccatgtggttgctggga
tttgaactcaggacctttagaagagcagtcggtgctcttaaccaatgagccatctctcca
gtcccagtatttattttgaggcaaggtcaaataatgtagttttggatggcctggaacttg
ttgaatagaccagacagactaaccaggagctcacagagatcctcctgcctctgtcacctg
agtgctgggattgaaggcttataccacacacacactctctagatttgatccagacccttc
ctaaagatacttcaccacaggggcctattttcttgtggcagtgccacacagccctctgcc
tggtgtagaggacccattgtcccccagcagacaatgcagtacggaccttggtctcctctc
cagtatgaactctgtgggaacaaatgcccatgagtgagtgaatggaggcattatgggtgt
ccccaagtcccaaagactatttctctgtctagccagtcctgcggaagagagagagccatg
ggaagcacaggggagat
Mapk14 (SEQ ID NO: 5):
tgtaccttccccttccagtagtgggtattgaacccagggccttgcaaatgctggtcccca
gcctggaagtggccattcatgatgtctacctctgacctcgtgcattggaactccctgtgg
gtccctggaacctccttctgccacttctgcctgaaaacatgtaggagcgcctggagggag
agcctgggctttgaggcaagactaatatttgcctagggggcaggtttcaccagcatcccg
aggtgagccacttgtccttgagcagaccggaatgcgacagctttctggacagggcctgtc
tgttctttcaagttgttcttggaaattgacgactctagtgagcaggttgctcggagttca
aagatcatttggctctcttccccgacgctgtcacagcggctttccgccatggcgccaggc
attcgcccgtgcgaccacagagaggcgcgcgcgccccgcgctgcggcctccacccttggc
gccccggggagcccagtccccgagagttcctgcctcggctccccaagccgcgtccctctt
ctcgcgagatctgccggccctgcgcgctcttctcgcgaccccccgccccgtgggcaggtt
ccatattgggtaagatctggattcagagcggggcctccttggagctgttctcgcgagagt
tccgcgagaggctcccggccgctgcctgtgggatcgccgccactggagcccaagcggggc
gctgaagcgcgagcgggtgtcttgcggcgtcggcgtgcgctccctccccggggagcggct
gcaggaggaccgcggcgggagcagcctcgagccgtgcagccggctccggcaccttgccga
cgctcgtaggagccgccgcggctgacaggggcggcgggtcgcagcctccacacctgcgcg
ggtggcgggcgcggggtccggtctgccgcgggcgggcgcagaggagagcgtgcggctgca
ggcaggagcccccgctcggccacctcctcgccccgctgctgccgctggaagatgtcgcag
gagaggcccacgttctaccggcaggagctgaacaagaccatctgggaggtgcccgaacga
taccagaacctgtccccggtgggctcgggcgcctatggctcggtgtggtgagtgtcgccg
ggcctcgggttaggggtggggtggggggacgcggtggagcaagcagggtggccctgcatg
cctgagggccaggcctgtccccacagctcagcgatgcgcccagcggcaggaattctcctc
cgggcgtgtctgggggtgcagggggcagctggagcgggacgtgtcctgcacccctcgccc
tgcatacgcaggtcgggggcgagccgtgtgcgggagcgggtcagctgcggacgcggtggg
atgcttctctaccctgcgctctccctgctcccaagactggccctcccgggggtcctctgc
tgcagcgcggcctcaggaccctgcctccgggtaccgccactcgggacgcgggagccctgc
cccggggagcagacgagctgggcggagctccagcgaggagctgtagggcaaagcctgacc
cggggactgcggtcacctgaacgaaacttgcaagagaaagcgctcctttcccggaatttt
tttttttttccttttttctcccccttttcccatttttgcaagcctccatcgaaaaatcct
gtctcaggaaacgttcctttgggattcgcacttgaattagcagcaccccgaagagcttaa
aataccgactttatctcggggaccacggtgccagcttgcgtggtgtgtttccgattcggt
tgaaagcgtatttccttttcggggagggagggtggcagcgccttgcaaggacgctccagc
tcgccgcttagtcacataccactgctcatttcagtattgtttgacaaaacagttttccat
accgagcagaggggcgcccctcaagatcaagaaggtaggactagagctggaggtgtagat
agcttttaattgaagtaattcatgggggtgcttttcttttttttaagtcccttagaagat
ttgcggagttttatcagcttgtaagtaccgaggtacgtggtggttttcgggagatcctgg
aacataggaaacgatgctttttagctttccttaacaccgggtgggggaggggggtaggag
tgggggggggcctattttagttccactatggctctccctgtcttgcctggaactcaacaa
attttgtctgccagctctgagaataaaggcttttgctaccatgcatggccctgggagcag
gtttcagattaaatttctaccataaaacttccactccagaaatgttctaaggaactttga
aggattcacgcagtatcttttactatatcttttagagctccctatctgatagtaatctga
tagattcagtagatgagcccttcctgtgttttagtgacttacatgcattcatggtttccg
aaggagtctgggaaatggtttttgttttggaacaaattccaggcttctggatgtgctaac
ctttaagtatgagtcctgtgttacttaaagtcatatcgtgaatttcctgtttggtagaat
cttctgggtaattgagattgtgcaattgtgtccagcaacagatcacattgcatttgctgg
ctcaagctgcttaagctatgttgcagatgataccaacttggacatctttctcacgagtct
acagaatggcagtttacactttaaaggaaatgtcaataatatctctcatgtatttgtgat
tgatctgttgcatcattgtttctctaatagttaccagctccttaagtatctccattcttc
gttcctaattggtacagtaccaggcgtttgtttccatttgaatgctcagggagtgaagct
caggggcttctgggatggtgaatttgcaaggtttcctgtggacaccactgtcctggcaga
agagccagcttcacagtgaagtgccacagtgaaaccaactccacttcctctagtgctgct
gttttctgtgtaatataattgagcacaacctcacaattaaatagacttctaccaaacagc
ttcttaaacataggtagtgctggcccagtgatggaacgcagtgggacagccctggccttg
cctgtgggaggccctgcattccagccccagcagcagtgcaccacacaaagcaggttgctt
gaaataagggcccaaaaggtatgcagcaaaaatcctgtttctgctctcagccctgcatgc
tggtcttccagagacaaccactgtttagttccttgcctgacattccagagaggtgtagtt
tctagcgtagagatttactatgtagcctattgatgaggaaaagatagctgaaattgtgtg
tgacccagcaaatgtcaccttcagttagatttgcaagtgttaacttctctcaagttattt
gaaattgtgttttcaattagctttttaggtgtgactgaaaaatacttttttgtttgtttt
ttgagatgagctgtcctagaattcacattgtagaccagactggcctccaactccgatcag
ctggactttgcctctcagagtgcctggattaaagacttgcaacactacacctgacttctt
gaatttcttttttaatccactttgaggcctgtgttccctttcttcaccttcgttctctga
gatagtttgatgttggggaataatgaagtccatttgaatggatataattgagggggattt
aaactgagctcttgagaagagaggaggctgttctactggtgacagtggaataagtcagga
gggacctgaagtctcttcaaaaaaggaatctctgaattggacatgatacactttgatttg
taacttaatttttttcttttaagtttaagattaattttaatttgtattatgcagtgagcg
acagtaacaaaaaacaagcagcaaactgtagtattaccaactgtgactcaaagttgaatg
agagtctagaaattgagaattggcttgtttttacaaatatctttctttttaaaagtgtaa
agggaactaaactaagttctctaagttttaaggaactgtatttactgcctgaaaagggaa
acaagcaaaatcattaagaccacattgttgcaaagcattgattgtgtggtttgggacata
ggggaaaatctttgtggtgaatttaaggaaatacaaacgggaacgtcagtgtctttcctc
tggggaaaggggctctccagccccttgctggacacacttgggtaccatgccttgggagtg
agaattaagaattacttctagatcattcacagctatttattcattagtctggattacata
acaccaaactctggctagaatcaaggctagattcttgagctaaaagtacagaacgagaca
gctcattttgtcattgggcttgtagaagaatggatggatttaatggctgcattttatggg
ttgcttgctgtgttgtaggcatggagatgcttagtttatgatcggcatgtagacatgtaa
caaacttgtagctaaacatggtaatttcttctaactaaatctgatttagcatgctatata
ggacaagattattccagtaaatataacaaaaataaatgaagctttttttttttaagtatt
aatagccattcaccattagttttaattagccagggaaaaaaaataaaactccacaattag
gtatgacttttcactcttgacgaccgagaaggctgggcctttactattagttgtaataat
agccggtctctcttccttcccttcctgcctttcttcctactttccttcaggacacagcct
tctggtcttgaactcttgatccacttgtctgaggctctctaatgctagggtcataggagt
gggccaccaagctgctttagctgacctatctcccatttactgttttgtgactaaaagtag
actagttaactggatgttaccagtcttaataagcatttaggtttgaggccaacctagcct
cactcagcagagtgaactcagtgccctttctcaagaaaaacaataaataaacaaaaagtg
cccaccaaatatgattggcaagagggtgtgtgtgtgtgtgtgtgtgtgtgtgtgtgtgtg
tatgtatgtgtgtgtgtgtgtgtatgtgtttagatttatttattgttatttgtatgaatg
ttttgtttgaatgtgtatttctacaccacaaagtgcttggtgcctctggagcccagaagg
gggtattggatcctctggacttggagttacagaaggttgtgagccaccagaagcccttgc
cagtgctcataaccactaagcatctcttccccagctcccggcaatggtttgtatggatgg
aggagagttcagctgagctagctgtagagtttattttgttgagttccgtttcccactcat
gatgagcactacaccccaggcctctgagttctgccacttttcagttttgaatattgagga
gtattcttttgatttctctttaattcagcctcagagatgatatgtgttggctttgccttc
tgtggacaatgttgggaattgtgctaaacccttaaagcttaggactcaaatgtcgaggag
ttccctggatgctgtcactttcgtattaactggcatcttaatgtgattctttggtttatc
atgatcttcatttaaggcattatcagtccctctctttctagagtaggtgggttgcttttc
cttctctctctcttctgcatctttagtaacggaaccctgggggctggtgcacactaacca
tgggatccagtgagctgcactccagcctccaggctggcgttttagcctagttctgtggat
gagctggggttaacagaagaccctgtgctgtatgaccatcagctagagctgaatggatct
gggagcaatcagaagggggagggtttgcagctgctaggaatcctacctggtcatatttgc
ctttgtaaagaccaacttctgtgctgaaaaagtaagccaaggtgggttgcagacagagca
gagctggggtctagctccctagcttttcatttctgttccttaactgatcgaggcactaac
taaactgtttgtggtttatgtaagcacaagagagataaatcatggcagggacttgaccta
tgccctgtactcttcatccacaagtccaaaagtgccagagccagaatttttgttgagctc
ggaaattcagggtactccacttcagctctctaaataacatgcttacttcaaaattttttt
ttgttttttttttttgtttgtttgtttgttgttgaagggttttgctgccaaaaggaaggg
atcaaagagattagctgctgagttcagctgaaaaagaaagaggagctgctggcaggctga
gtgttttctaacatctttgacttccaggctttttcttaatacttgctcatattttaagtc
acttgtatcttcctcagagtatccgagcatttaaaatcacatttatcttaatctttggtt
ctgtgtctgttttccattcctgcctttccctcattgtgttacttggcactgatgtttttg
tctgtaagactccaaggaatgttaacttgaatagatgtgaccaatacctgtctacagata
ccttgtaacagctctgctctgtcctgtcttggctgtcttcatgtgctgcacactctctgg
ctctcttgctctcgctcgctcgctctctctctctctctctcgctcgctctatacacgcac
acacacatacacacacacacacacatcccgcctacacacactgctttatcatctggttgc
agaaagtaagttcctgaaaacaggttcccatggcaacataacagaaaggtaatagaacaa
atgtttattctgtctagacctgttgtctgactaactgtagaccctgaacatgtttgctcc
tgtcacactgaacaatgtgattacagatttccttgtttccctcaaaacctcctccttgta
gatcttagtggggttgccactgtgcttgtgacgtgaaggaagctgtgtgtgtgccctact
gcaggtgtatactggttgtgtccccaagcttgcaggccacagcttacctaaaacagcagc
agttgctcctttgttgtcataagttctttagcttagtattggtagtgttaagcgttgttt
ataaagtccaaagaaagcaagggacctggggaggtgccatagtgcttgccgtgaaagcat
ggggccgtgacttggatctagacagcatgtgaaagccggacacagcacatgtgtgagctc
agtgctggggtgcagagacaggaggatgggtggagcctttggactagctggccagctagc
ctagccaagttagtgaggtccagattcagtgagaaaccctttctcaaaacacggtggaag
gcacctaaggtcaacctctgacctacacacatgcacacacaaaagcaggaagagacctag
gttgctttggaagtctttgtagcttttctttctttcatattgttcagtgtcatcatctca
aaactacctttatatttctaaagacagcccttcccctttcaagtgacattctagtagctt
gtcaaatctagggcagcttgatgccagggtatgtattggaaatctgtgtcagagaaccat
ttccatctggtgattcagatctggaaaggatgacatcagcgtgtgcccacaagaggtcag
aaaacaaaacaaaacctcttttgatgtgtgaaggctctgggaggctgttttagttaaatg
gggtgtgagagtaattggtcttgatcttgagccctttgctcggatccacttggaccattg
tcatgctcataatcaggcctttgctctttcctgtaacacgcagcttggggtgtctggctt
gtgcgagtactgagttcatcttgtattactgcattcctgtagcagatctaatttaattcc
cagaaataatttttttattcttagtatcttcagtaggtgactcctctttacccttccccc
ttaaacttcacttagggcttttgctagctctaacttccttgaacctaggtttagaaatac
atcgtttaatctaccagttgccagtgaagactttagttctttctcatctgtaaaaaaaga
aagaaaaatccaactccctagtaatcattgtgcaattctgatgttgacatcttgtgttga
catagttgtgattacctgattgtgtgtgtgtgtatgtgtgtgtgtgtgtgtgtgtgtaaa
tgttcttgcatgtgtgtttgcatatatgcaaagaccagaggttaacttcagttattgtcg
actatcactttctactttattttttgatagtatctctcactggatctggagtgcatggat
tgcttagactagctggccagaggtcctcaggaatcttcctgtttccaggcacatcgtctc
ctacctgttttttgttgctgatgttgtttggtttatgttgatactgggatcaaactcagc
cttcatacttgctgctggagcacttttctgactgaactgtctccccagttctgcccttgt
gtgatgctgcagtctggcttgcttaggtttctgattccttatgtcgtgttctgagtgccc
tatggttgggtgatctcttttgaaatgagcgactcaatgttaaatgatactgataccagg
caaaaggtttaaaaagtaacagtgttatctgagatgttggaaaaatagttgcctggttaa
gaacactggctgttcttccagaggatctgggttcaattcccaggacccacatggcagctc
acaactgtctctaattgtagtcccaggggatccaacaccccacacagacttacatgcaat
acacatgagagttttaaaaattgtgatctgtttttttttttttttttttttttttttttt
tttttttggtatctctgaacttctttacatccccaaacatggtgtaacatagggttcttg
gtttgtgcctgttctgagcagagggtgatactttgttgtgggtctgactgggtattatat
atttaacagcagtgttgtcccctaatcttaagatttatgacaatttcccattgtgacagc
cagatttgtcttataagtggtgccttatgttgggggagggttggaaattagtcaccttta
attgaaaacgttttacacaacagtacatatctagaataattggtcttgactaaaacctat
aggctaacttgaacactgtacctgtcagcagagaggaatgctacacatggggcttccttc
agcatgaccgtcattatcttcaggagaacatgagaaagcattatcgaggtcccaagctta
ctgtggttagaaccgcttgcacagatcgtgtgttggccacgtgggtagctgtatagtgct
gtgaagaaacaatgaatgtgaataactttgttttccttttgtcgtgattcctcttaggac
ttgtaaatgtttctggaaaggaaagcttattgagtatgttagcattaagtccaaatctga
aggtagtgcttttcccagcatctctggttttcttttcttgactttttttttttttttttt
ttttttttttggtttttcgagacagggtttctctgtgtagccctggctgtcctggaaact
tactctgtagaacaggctgggctcgaactcagaaattcgcctgcctcagcctcccgagtg
ctgggattgaaggcgtgtgccatcatgcccggctatctctggttttctcgccctttttct
cctactgtaatctttcagtaggtagctcttttcagagttcaaatatacagtgttcaggcc
agccaggactatatagtgagaccctatctcaaataaaataaaaatgttagaaaagcaaga
gaatttcatgtgaacacagaacaagaagtagcagtgttacccattgacgtgaccagctta
tgcttgacacagtccagatatataagccatccctggcttggagcataccatgcacactag
cctggctgcagatcatggtagtctccttctttctacctctgtctctgctgggattacagg
tgtgcaccactgctcttattttgaaggttcttgttagcattttagtctttaaagtattag
acagttacagagggtgagtttttagtaatctgtggattccagcagtaacaaaaaaattag
ctttattcttgactcagagccttccatttacactcatattttggtgtttcctgaattaac
catggtaatgcgtgaaaggttctggcatggtaataaataattctgtattagaatttgtaa
cttttaatatttaatgctgtgctttatatctttatttctatgtatatgtgtgcctgttca
caagtttgtgtggaagctagaggtgcagtctatcttccctcagacagagtgtcatcagcc
tggatctctagttaggctaactggccaacaagtcccaggaatcctcctcttctgtctcta
cacagtcctggagttacaggtacacaccaccacacccagcacttaagtgtggttctgggg
atagaacttgggttctgacagccccactttgtgtcgtcctcatacttcaaagtgctctgt
tgttctgatatcctgtgtttatctttgggcagttttactcatcatcgccatcatcatttt
gcatcatgactcatttttaagttttctttttctatatatatccattttgggcacattgcc
gtacaaaagtactcttggtggttggcactcctctgttgtgctaggagtggtggtacagac
cagtaattccagtagtcaagaggttgaggcaggatgattttgagtttgaggtcagcgtgg
ggcaccttgtgataatcttatatcaaaagaactagggacatctaatcttgtaaattccga
gtccactgagatgtacaagattttgtagcaccctaccttccctgattagtttttattttt
ttaatgatttgtttgctttctgtgcaatggtgttctgtttgcatcatgtgtgtctttctt
tcttttcctttgctttgatgaagtaccctgacaaaagtaacgtgcaggaaaaaggtttct
cttggctcacctctgaagaatacagtcctttatggtggtggtggtggtggtggtggtggt
ggtggtggtggtggggttaaggcaacaggagcctaaagcagctggtcccagccagtccac
agtcaggaagcagagagtgatgaggtcatgctgctgttcagctccctgttcaggatccca
gccagggaatgactgggtgttcccagcccagtcagtgtaataaagataatccctcacgcc
cagaggcctgtctcccaggtgagtctagatcctgctcagttaacagcactaaccattata
gtgtgtgtgtttatacacacttaggtttaagttttcgatttactgtagtgaagaaaattt
gttgatacctatcagacagtgttccagactcaccagcatgtggaacagactgcttagaaa
taaagaaactgattagggaactatattcacatagtgtggacataaacccagaagtggtgg
ttgtctgtgcactatagtatttaggatggtttcatggaggaaaaaagtattgagagttat
tttgagagctgagtagactaatcagtacagcaggagagaatatcatgtaagtgaacagat
gccaagaggaccagcctgaccccgctgcggctctcagctgtgtttcacagtcactcggga
gtcaagatgtaggtctacaattggactcaggtaactgctttccctaatagttgacttgtg
ttcctgactagcagaacttctgtacaaagcagagagctgttatcttcacttatacagtgg
aagaaacagtatgcctttaatcccagcactcgggaggcagaggcaggcggatttctgagt
tgttccaggacagccagggctacacagagaaaccctgtctcaaaaaaccaaaaaaaaaaa
aacaaaaacaaaaacaaaccaaaaaaccaaaaaacttcctgtttgaagtgttggcttgga
ctgattggaggaagaaagggctgctcttgtacagtcggctgcagtttgtctgcaggcacc
cctgtctttttgactccccagccttctctattcttgaggtcagcagtgatctggttttgg
tatgtgtttgttatttttgttttatgatcaggtcatggctaggtgccaaggataaaagat
aaaccacagcttctgctctctcacagactggagtaggaaacaagtatgcagaggaaaggt
ggttctgagttccgtctttaaaggattagttacaatgtactgccaactgtagtagttctc
aggttccatctaagggttgtagaatccatgtacacttctaacttgtagaaactacaggta
tccatagattgacaaatcatggaggatttcaaggactctcagttacattttaataatctt
ctctttttactattacttcagagatatgctcaacttgcagtgtgagagaaagatctaaaa
atactgagagtttactacatgggaagccccatgctagagactgcaggcacagacttattt
tatagttggatattcatttcaacttggagtatttaaaactaccttctggggctggagagg
tggcttagttgttcagagtttgaactgttcttgcagaggatccaagttttattccagtag
cttataactgcctgtgacctcagattcagacatggcctctacagacatggtactcacatg
cacaagcccccacacagacacaaacaacagttttggggttttttgtttttttgttttttg
tttggagaggaaaggatttattcagcttacacttccacattgctgttcatcaccaaaaga
agtcaggacaggaactcacacagggtaggaacctggaggcagaagctgatgcagaggcca
tggactgatgctgcttactggcttgcttcccctggcttgctcagcttgctttcttttcct
tctttttttttatttttttatttttttaattaggtattttcttcatttacatttcaaatg
ctatcccaaaggtcccccataccctcctccccactttagtttttaatataaagcacattc
aacatacttacttgagcctcttcacagcgttttaggctaggcggggctggtgattccttg
ttttattcctttggaaacagatgcgttaaagctttgtttgggatcatctcctagtaagtg
ccaaactagaactcatattttgactactggtttgttttttgtttgtttgtttgtgtgttt
gttttttgtattgcatggggtacatttaaatttgctaattggaaatttctagatcaataa
aagatttttttttttttaagaaaaagatttcttttatgtgcattgatgttttgcctgcat
gtatgtctgtgtgaagatgtcagttcccctggaactgaagttatagacaattgtgagcta
ccatgtggctgctgagaatagaacccagatcctctggaatagcagccagtgctcttaact
gctgagccatctctccagcccaataaaaaggtttttattattattattattattatttat
tttttgtatatgagtacactgtagctatcttcagacacaccagaagagggcattggatgg
ctgtgagccaccacttggttgctgggaattgaactcaggacctcaggaagagcagtcagt
actcttaaccgctgagccatctctccagcccaaaagatttttttaatgtagagaaataca
ttagaaataccagctttgggctggcaagatggcttagagggtaagagcactgactgctct
tccaaaggtcttgagttcaattcccagcaaccacatgatggctcaactacccataatgag
atttgatgccctcttctggtgcatctgaagacagctacagtgtacagtgtataataataa
ataaaccttaaaaaagaaaaaaaaaagaaaaagaaatactaactttgacttaacattggt
tatggaggtttgtttgtttgtttgtttgggtttttgttttgttgttttgttttttttttt
ttgcccaaaagaaatccaaaccaaaaaacccggtaagttggaatattaagtcatagtgct
tctaacacatctatccaccccagtgtcctgccttagcaacagattacactgtagtgtgtg
aacacccggcttcctgggacagatgctgttgctgagcattgggagagcatctgcctgctt
atcatcagcctggagacgctcacgtcagaattccaagtaatttggtactgagtgcttgcc
atcttctcagtcttaaaggtgtatagaacaaagttggagagcaaattagagaacatctgt
agtctgaataaagaattaagatgtcctgttgaggcagagcttagttccttcccctctact
gaggactgtacttggtggctcagttttctactgtgtagaatgtggcaggaatgagtggct
gcagaaggaatgctgtaaatgagattgtacctcctgtggtgctttttccctgggaacact
gctgaaggagtaggatatccaacatcttgcttacaggctgctttgcaggccatgtgcatc
aggatagctatgaattccacccaacacatgcatagatgacatcattctatcacagttcag
gaggttggacacccctattctagagcatagtttctcagctctgatacactttggcatttg
gggtcaaatagttgttcataattcttggctgttgtatctcctgtaggatgcttagcaagc
agcatctctggacaccacctacttggtgccaggaacattcttctctaagacttggttgtg
acagtccagaatgacttttggatgtcctctagggaacaaaatcaccctttcttttctttt
ttaaaatagtatttatttcatgtatatgagtacactgaagctctcttcagacacactaga
agagggtatcagatctcattatagatggctgtgagccaccatgtggttgctgggaattga
actcaggacctctggaagagcagtcagtgctcttaaccactgagccatctctccagccct
aaatcaccctttctaaagaacttctaggggaagcctgcaaagcagccctgtggagagccc
aagatggcaagcactacagctaagagaacccttggaaaaggatcctccagcctcagcagg
tataaaagtgactgttgggctggagagatgttcagcagttaagagcactggttgcttttc
cagaggacctaagtttggttctccgggcccacatggtagctcacacccatttgtaactcc
aattccaaggcatgtgacttcctcttttgactactgcatgaacatggtacacagatatac
atgtaggtaaaacacgccttcccatgatgaaaatgtaaaaacaattagaagagacttgct
atggtccatctcttcactgcagctttcggggaaatagccagagccaccttgctaaatagc
ttcctagttcttgatccacaacaataggagataataaatcaggtttaaagctccacattt
tgggaatatttgttacccagcactagataataccctaagtgctatcataaaatgtccaga
tattgtataaggtagaaagttctgtacttcgggaagttagcttgtggagacatcagtcct
tgtgtttacccatagttaggtttgtagctcagatttatacagagatggagcctaggaact
attgctatgaaagctttttccatgctggtagctagaccatagtaacttgagtgtaatatg
gttagtctgatattttagatttatttgaagtgagttaattatgattacttaaagaaatat
ttgctattgagattttgaaacagcaaaatggagaaatgtttctagtggtacactgtaatg
aggctttggaaaatagccccttttataaaagaaggcacagtgggagggacataacactca
aaaatgcggtttatacctgtgtggaaatgaccttataaaacccctgattgtgctggagtg
gtaaaataagggcctttacatttgtctgaggctagaggatcctctaaaggcaccttaccc
gtgtggctattgacagaaggcctggttactatctagatctctgcacagggctgctacagt
cttctctagatcagtggttttcaacagaggggacattttgttcccttggaaaccacattt
tgaaagggagggggaatctgggtgtcatggcatgcacttgtaatctgagcactggggcag
cacagaagggcagatcttgggcttgcccagtccagcctacttggtaagttctaggccagt
gagagaagagaccctttctcaaggtaccttttcaatttaaatgaactagacagtgccaga
atagtagtatgtggcatagtcacttgttgagtggattgtagattcctttcctctgggctt
tcagtttgttttttagatggtttaaggcttttctaggagtgtctgcttttggaaatttat
aatttagagcagaatgcatgatttctgttctttccaaatactgctctgatagggagagga
agcagcatgttagtgcatagagctctagggcattagcagcctttgctggagggtttccaa
cccacaagaactatgtctgagtaactcctgcctgtcttgtacatgtaaggtgtattttca
tgggagtgtgtgtcctctatggtaagaattagtatgttgtaatgagagcatgtgattagg
gaggaagcattagacggtcactgagctggtgctgcgttatatgtgcggacatctggtgtg
actgcatcagtgagaatatcacaaaaggcagatttgaaaattacttggtaaaatgaaaaa
tgattgtgatttaggaatgtaggtttcagatttacatatggtgattatgtccgtgtaagt
atcattagaaggaaatgtaattgctactaactagtagtttgcactaagttgaaaacacca
ctaagtgacagagcagcagtgtgagtcacgtatctcactctactaatggcctccatgaga
aatgaaagcttgttttaagaaaaaaagagagagagaaaatggaaacatacactgttcaaa
agagtgtaacaagtttaggatatattagatatttatgctgtatgaatacagtatttccct
atgatagaatcattttctgcttctgaaacttgttcttttgccctcagtacatgaaagagt
cttgctctttgctccatgtgtgtgaacggagaatgtactagaattttaaagtcctgttgt
tggaatttcaagtagtttctagttttcctgtgaccaggctgcaacagatatgtactttga
accctattttgtttgtatctgtgccgatgttttgagatatgattttactctgtagtagac
tggcttctaccatgtcccatctgcctctgtcttccaagcagaaggattataggcatgaac
cactcactactactcccagcttcagtgtgtgtgtgtgtgtgtgtgtgagagagagagaga
gagagagagagagagagagagaggaaggagatccttttttaaagatttatttatatgagt
acactgtcactatcttcagacacaccagaagagagcatcagatcccattagagatggttg
tgagccatgcagttgctgggaattgaactcaggacctctggaagaacaagaagtcaggaa
aagaatgagagaatgagaatgaataaatgaatgagtatgcatcattacagacttagactt
gtggatgtcggaggacaacttgcaggagtcagttctctctccttccactgtgtaggtttc
cggggctgtattctcaggcctggcagtaagtgcctttaccctctcatcttttctttacct
gctgaaccatttcacaggcctcggtatatatggttttttaatccaggatttggagaaatg
tttaactgtctgttggctgtttaattggtcattcctggtatcttgtgcattttgttatcc
caggtagatagtaagttttgtgacatcagctaccatatttactgttttcttttgtattca
taaaataaaataacatcgcttggtggtaggggcactcacctttaatccaaggactggaga
ggcagaggcaggcggatctgtgtgtccaaggttagcctggtctgcagagtgagttccagg
atagccagggctacacagagaaaccctgtcccccaaaaacaaaaccacaaaaaaagaaaa
aagaaagaagccctataaaccaggagtggtggcatgtgtctctaatcccaacacacagta
actgacaagaaggtcatacattcaagccagtgtagaaaatttagccagacactgtctcaa
aataaaacaaacattagtaaagggtatgcggctcagcttactgaccagtatttgtctggt
gtgtgtccaaccctgagggaactgggaagtgctaattgaggaaactggggttttgagccc
tttgtatcacgatggtgagggaaacagatggacttcttgtcctcaacttgcccagggcag
gtggcagtaggagtcattattaatgtctgataattgagttttacaaaatttgctaaacct
gccataaaaactagtaccttaggttagatacagacattatatttgttttatatatgaggt
tggtagaatttaattaacttcctcaagagttattaagtggcaaagctagtattaaaaccc
tgatagtcctcttccagagcccagcccttgtttaactacctggtgggtggtaaataaaca
tatactaatcattgtgattattggggactgtagggtccctgtgcggcatctgtcctccac
tgagtggctagtgaagtcagagggtggaacctgcccctcttgctctggagctggctgcaa
aagagcctgggtagtgatcataatgggcagctgttctgctatccagctgagctgaacttg
taggtgtggccacatcctgctggccctccctgctccccacaggttcatccccaaggagag
gacagagttacaaaggctgcatgaggcatgacactcagagtggaagttgaagaagccaca
ttgtgtgggggccatggccatttcctcagtttctaaccacacactggtaaagggaaggtt
gtcttatggtcttatggagataaagggtgaaagacggagagtgcttgcctcaggtgggta
ggaacccctgtggaggggtgactctgcctatatatacttgaagaataaagatttagttgt
ggggtgggtgcgggtaggcgagaaggaactagcagcagcagatccaaatagtttacagaa
ctgaaagaagccagcacaccagagcaggcctgtggtgggggactactgattgggtgagcc
ctgggattgggtcaactcatctgaaacttagacagcggtaggaaatgtgcatttttattc
tgtattacaagaaaattccttgagtttaaaatagtcatttattattttgaaattgcacgt
cggagggcagcttgtaggacggatcttttcaccttgtcaagttcttaagaattaggtatc
ttctggaccgcccacagtcctgctgcccagaaaagagatttcaagaaaggataggctttg
ggggttttgtgtttatagatggaatttaacccgaaatgaccagttgtgtagttttctcat
ccctgcaaccaaacacctgagaggagaaaggagccagggcttacggtctccatttcacag
ggctcccccgtgcttgcctccatccataccctcgagcagagcacgtgggagggcataccg
tgggctgttctgtacacctgagctggcaggaatgggacagaagagagggtgcccaaccaa
actatctccttccagctaagctctaccaccatagtgaaaacactgaagcttttctctttt
gtttagtatgaggtgatgtactgtgtgttcttgtgtcgtggtgtgcgtgtataggacaga
ggacattttcaagttggttttcgccttttaccatttgagttcctggaatcaaacttaggt
tgtcaggcttgtaccctcatccactgagctatctgcttgggccctcccacctccaaagat
tttagaattctctcaatctggaaatggcacatctgttgttccagtgttcagaaggtggag
gcagaaggatcaggaatttaggattatcttcagcttgagagtgagtgtaaggccaacctg
gatttacctaacagcaaagtggtgctaccagctgggaaccaagtgtttaacccatgcgcc
tgtggaggatagttcacgctgaactgtaacacagctgtaaaggaggttataaagatgaga
agatgaaagccgcccaggaccctcccctgaagaactccatcatcaggagcttatgtagta
caagtatgtgatgcagagaaagtcccagaaagccccatgtccattgtaggattgacagga
ggaagggggagtctgaccatgccaaatgctaggccagaacactggccgtctgacgtgctt
accgagcccgagcccggtagtgggcttgagaagagcagctccagcaggctggattgaagg
atgaatggagctgagaagagcattgtctgtctagctgtgcttctcaaacttaggaactgt
gtgaatcacattgtcacaatgcagactctgctctgtgggtcaagatcctgccttctcagg
gagctcccaggcagagccaggaccactggtctacagagtgcactttgaacagcataggtg
taggttaacagctcttgttttagttagtgcatgaaggctttgagaattgcgctctcttca
gtttctgatttgtagctctaggatggtgtgtccaggagtttccttactaattaatccttg
ggtgaagctgacgtgaatggttcccataccttgagaatttggagattattaagttttgga
gaattttttaaactgtgaggaagatcatcaaaactagttaatgaatatggaaactttcta
gagcacattctgagacaagtgagacatcagtgtgtaaataactcagtggtagagcactcg
cctacccagggttcagttccgtgtgtgtgtgtgtgtgtgtgtgtgtgtgtgtgtgtgtgt
gcgtgcgcgcacgcgcgctcgatgtgtgtccatgtgtatgtcccacgtccccctacgtcc
ccctcctcccctttgtgagtcagcaacacgcaggaggaatgttgttgagaagatgtggga
cggctccaaagcacagtcttgggtttgatcagagggcatgtgttccttagagtaagagag
actatggagagtgttggctgagggcatctggggtgcacagatccacagaagagatggagc
tatatggatctccctttgctagcttctgttctctcaaggaaagggagggagagaagggat
tggaagtttgagaggtggaaaagtcatgagatcttttttcagagtagagaatgaccttgt
gagtggagtcactgtttcagtgtttgcaattatgttcagaagtaaaaacttagaagcagt
ctgattgtactttttttttttttttcaaccgtagctagccttgggtaattaggtggttgt
ggtcacctaaggttagacttgatccaaggagagggtacaagggagcagttaggaggttga
agacaggagagctggagacacaattgatgaggtcagcagcctcgatgcactcaaggtaga
gtagtgaaagcaagccgaacggttggaggggcagtcggtgctggagagagatggctgaaa
tatggccctgcatgaggtggagtttccggagaggtcaggaaacgggatgttggatatgcc
ctccagctggatgatatagttgccaagaatgttatcaaaagttgggagtggaggggtact
tttctgataggacagacttttggagggtgatggaaaattctagtccatagataatagcaa
ccctgaagaagggttgctagcaagggcttttagaaaacaaatagaaagtgatgtcttgaa
gtgccgttaccgcgaagtgcctgttctgtgagagaaatgtctgtggtatagcctcagaaa
ggaggctgggaaattccaagagtgttaccacaaatcaaattgatggcgaggatatgtgta
ggtttgggggtaaaaggaggactcctgagcaggacagactggtgtgcaattgttctgcac
tagccactgcgatgctgcactggcgtcatgttgctgctcatcccacctgtgttcattcag
tggtcactgtgtgcatcactgttgtgagtgcttattctcctctgtcactcttcccacact
tgaggacttcctactgtttgtgcttcagtctctgggaattgtccagcacttggaaggcta
ttctcctggaagcaccgtaatttctggggcctttactttgaccagcacatagtctttaag
attatcagtgtgttggctccttggacaggctgctgcctggtttcatgaaatttactagaa
aacagccatacccattttataggttcctattatttggctgctttcaggcaacaactacaa
gctgaggagttgtgtcagggaccttacctgggctttggagagtaaaatgctactgtctgc
gcctctctggaaaatgcttaccaactgctgatatagacaaaagaggcagaaagaagcatc
tcaaataaataggtgccttctctgggtggtgggtgatgagtcattatagtctttcttttt
gtgtcttgccacaaatttaatttggaaatgaaatagcctgaaaagtactaaaaatagtat
ttggaacactctttttttttgtttttgtttttgttttttttgtttgtttttccagacagg
gtttctctgtatagccctagctgtcctggaactcactttgtagaccaggctggccttgaa
ctcagaaatttgcctgcctctgcctcccaggaggcagaggattaaaggcatgtgccacca
tgcccggcttaaaatagtatttggaacactcttaatggtgggttctgttgttttccttac
atcagtgctgcttttgatacaaagacggggcatcgtgtggcagttaagaagctgtcgaga
ccgtttcagtccatcattcacgccaaaaggacctaccgagagttgcgtctgctgaagcac
atgaaacacgaaaatgtgagtaattccttttcctgcacacactgaaggctttgtatgcaa
tcctgccggtggatgcatgtcacactgtcagagttgggttcatgccgcatgagggctgca
gctttggtgccatggggctgtcagatcatcgcgaggatgaccgagtgcctcttcccagag
ttgtacttttactacaggatctcaagaagcatttaattttctatttttatttagttagtt
tctgtgtatgtttgcctgctggggtttatgtgaatcatgagatgcagtacccttggagac
cagaagatgtcagatctttggaactggactcacagatgcttgtgagccccactgtgggtg
ctgagaaccaaacctggaggctctgcaagagtaacccccactctttgcttagatattagt
gtttggtgtgttttgtttgttttgtttttttctagacacagggtttttctctgtatagcc
ctggctgtcctgcaactcactttgtagaccaggctggcatcgaactcatagagacccacc
tgcctctgaacttcaagtgtttgaattaaaggtgtgagccaccacaactggctgtgattt
gtatttttaacatgtatgtttgtttgtttgtttgtttgtttgtttgtttaagacagggtc
ccagtatgtagccctgattgggttggaattacggagccactctccagccctgtattgctt
tgtatttttgaacaaagcaggcacggggggaatgagaaaagatcagaatacttacactta
atgtagaaattatctcacctttcagtctatcaatattatgcagtgaagaacaaagacttg
ggactgaagagatgtctcagtgcctatgcgcatttgctgctcttgtagaggacccaagat
tggttcccagcacctacattgggtgattcacacctgcctgtaactctggcttcatgggtt
cagtgccttcttcctcttcatccagggatacacacacacacacacacacacacacacacg
cactcacgcacacacatgcaggcacataggggcaggggagagaaagacagacacacacag
agaaagagacacaaaataaatattacataaggcaggaatcccaattcttttttctgtctg
atacactactggctatgttaacaaattctaaaaagatagacggttttttcatggtattat
gtgacgtgataagcaggtgttgtggcacacatctgtatgaacttgggaggtggcagcagg
aagatcaggagtccaagttagcctgggccatatgaaatgctatctgcaaacacccaaacc
ccagaaagaaatgatgttagcaaatataagacagaaacacagtctgggtagaaaatcagg
acactgagcacatgggtgtggctgcacttggagttagttctaactaaaacactggagtgg
tactgtaagaagaccactactttcccactgtttgatttttatgtgttcttgcaggtgatt
ggtctgttggatgtgttcacacccgcaaggtcactggaggaattcaatgacgtgtgagtg
aatcttctgtgtttgcaggctcagcaagaggatggggatgagcagcccatggaactgctc
agcagtatccaacctcagaagattattctccacctattctggtttcttgggggagggata
cataaagtgaatattatgtatggtgcattaggacacagtgtggcccattcagagccaatg
atgagtccttacaaaagttacacagagagggagtatagggaattttggttataattgttt
ttccataaatgcaagtccacataggtgtttccttctttgatccctcctgatgccaggtga
tactgaggctacatactgatacttaatgacatcttccagtctgatgccttctagaaagca
gagcctggaagtcccaggaaccacaagtagaggccaggagaaataaaacagggacaaagg
ggatggtaatgcatggatgtgttagtgagagcacctggctgggaactgccagggtgtagg
agccctacagaatgcacctcggatggggctgttcttctgattgcaactaaggtttgctcc
attgttcatgaatgctttgcatgctgacacacatgtgatgcaagacagcaggtgtcttgt
gcaaatccagccttctcaccaccatagctttcctagtatgattaacacccgtattaatta
tcatcttgacagaagccagaatcacctgatgggcctctgggcatgcgggggggggggggg
ggggcggttgtcttgactattaattgacaagggaagagccatctaaactgtggttgtgac
tgttccctaggcagggcatctgcagttgtgtaagacagaaaaggcagcagagcagtagca
tctagttcatcttgcttgctggtgcagtgtgatgactgtgtacagctgtttcccttaaaa
tgcttatgtcagcttgtttatcaaaagaagaggaaaagacacagccccaccccacctaag
taagagacacactgcgctgcagaaggggaaacatcttaagcacagctgtacgtttggtct
tgttaggattctcgaattcctaagtgcaaaggatgtttattgtaaaagctaaactgtagg
tcatcactgctcttccctaacttcttgtattactaagccacctttctctacgacatgacc
aaagaaactagcttatgtaaagagaaaaatgggttccttacctcctggttttaaatgttt
cacatcatagtcagatgagcccatggcttctaggcttctggtagaggtgccaggtagtgg
tagagagtgttggtataacagaaaccactcatctcatggctaagaagcaaaagaaaggac
ggaccaggatttcacagtctgcagtcacacccctgatgacttaagccttgccatgttgtg
ggaatttggaattttggtttcctcaagaaatgctgtgcttcacagaagtagtgtatgact
gtttttgttttagtcccgggtatggggatatggtgctgccgtgcagcagctgactgtgat
ttgccttgtcctctagcacagtttttttccagctatagatagtttctgcaattgtgtaat
gtttggaattctggaaactttttacaaagtatgtaagtgctagggcctcaatgctaggtt
gatggtgagtggttgtttaggagggctggttgcagttgctagtggcagtgctcaagaaaa
accagaagaaagaaattagattcagggatctctctctctccctcccaccttccctctctc
tatctagtgatgggggtgagcccaggggataaagggtgggaaaaagaacccacaaagtag
caaagaccggctacattacaaagaccggctacattattgcccagaggtttggctgctttt
gtagcacccttggaaaggaagccattgacatggggttgtgtggggttattgctcaccatc
cacactataccaacctcactcctgatgctcccagtgcaaaacctgcttctctccattctt
tctttggaatgtatgcccacaccatttgtcttttccaacagaacaagcagacagatacat
ttgctctcttgtgtcatgtctatagaaaagggaaaaacaagtgaatgccaaagatggagc
aggagaggagaggtggcagcgggagggaaggcagcttggcccctgtacaggagctcaacc
acattagctcacctgcaaatgcaagtttaggaagcaagtgaaaggaagactggcagtgag
taagaacagtgtccgcctgtcctgcagtcctgtctcagcagcagaacagaggctccaccc
acacaggctgagcagactacagagccctgctgcagccggtgcttggaaaagagaatgtgg
tggagtctaaatgagaaacaaatgctgctgcagtcttttgtgaattactttgaaaaacat
tggagttaggtctgagtgttaacctaagttttagtgagggtagactctaacagaaaaaaa
aaaagatttgtggactccaagtattgggttttgctttgattgagcattctggaatcaaaa
gtggacctatttttgctacattaaaagggcactaatgaaggctgatgtctatttgaatgg
ttaaattttattgtctaggtttaacccacccactaccagcatttaccccagtaaataaag
ttgtattcatttattacaactttaaagttagtcttttcaacaagagcagaggcttctgtt
tttacctctagctggtttttaacaactttttaaaagatttatttttttattgtgtatatc
tgtctgtacacatatgtatgtatgtatgtatgtatctggcatgtacatacctgatgtctg
tgtaggtcaggaaagtatgtcatatcttctgctagagttataggtggttgtgagccacag
tgtggagtcctgtgcaagagtagccagtgctcctaacccctgagccctccccagtcctag
ctcttgtttgagcaaagcagctctcaagtgctgtctggtaggtactgtgccctaagtgct
gagtacctgcaagtcagttaacatgctttccatgagaacagtggagagcctgtttcagta
gggagtgtttcattgctgactgtttgtgatgttgataccagtgaaaaggttagcaccctt
ccagaacagagcttatctctgtgttctccacatactatacgtcttcacacaggtaacaag
aatagtgagtgtagtgttgccgattgagcctcgaaaccatttgtgttcactaagaaaaaa
ttgtcgtgcaaacagcatactaaggattagagactaaggctgccagatgcatagcaggct
ccaggtctgcatctgcacttcccatgctagtgagcaaggtgtgccactggctttcctgtg
ctttcccgagcctgtgcgtttatgtttcagttactgcttttcctttcatcagatggctca
catttttgcttcatctgaaagaccacgcgtgtctttaagaaaggagcaatggactggcag
tggactgtttttgcttgcaagtcatggaggtggcttgggaagtgagcacatggaattctg
actgctgtataacagcagttgcaggacaggttgcacattacagagacacaaatgggagat
catttgagcacttctcctgttgttcttagttcttgcctgtactggtcagtgtacaggtat
cattgtgtatcctgaatatttacaaatgcagaaagtttcaaaagactatattactttttt
ttttttttttttttttttggttttttctgagacagggtttctctgtgtagccctggctgt
cctggaactcactctgtagaccaggctggcctcaaactcagaaatctacctgcctctgcc
tcccaagtgctggcattaaaggcgtgcaccaccactgcctcacagtctcccaaacattct
taaccaagctgtcttagctgagtaggcagggggtacttttaggactttttggaaatactt
tcttcaactttcaggaaagccagcttatatcagagtaagtggaaatgggacttccttccc
aacttacaacttttcctccttgatttttttttcccttccttttttccttttctttctttg
tttccttcactcatttatttgagacaggatctctctgtatccctggctgacctgggattc
ccagagatctgccagcctctgcatctctgcttcattcactctttatatttgttagtttat
ctttgtgatggaatctcctgcatcccaggatggcttcaaaatgactgtgtaaccaaaaat
agccttgaactccagatcctcatgcattttcctacctagtttatgcagtgccgggaatag
agccctgggctgcatgccgctgagcaagcactctacctccaccagcttagccatgctcat
tcctctgctcagtcctgaagagacaggaggagtctcgaagtctgctgtgatctagcggat
gcactgcggggcacttagcagctctcattttaataaagctggagagcagttggtcaccaa
gctggccttgctcctgtgcagagtaagcttgggcactgtggttcagttaactagaactga
gggcatcgagtcagcgaatacattctgccaaacacggtcctcatccttgaggaagacagc
aaacatgttctctaatcattgagtttaaatggccacatagtgtgcagtgtggtaggtttg
ggagacccagcatggtctggctctgtgcactctgaccttgtgtaacccactttccacttg
tgtctctctgtctttgtattctttctaatacagacaaccagccacatcccagcaaggcct
ctgcctgctctcctcacagcctggcctctccacacttgctgattggttcccctcagtccc
atacaaggttctctggatcagaacctttattcctctcttgtgccctagctgggctgtgat
ctccaggcactatctgtgccttgctctgcctggtactttgcaaatattgtcaccatatat
ttctgtctgttttctcccttgccttgtaacctctgttagagtagggtgctttcctttatt
cctaattgttagtgtttggagcagtagcaggcctacctgcagatattatttgtagaagaa
atgagtgcaagaactaacaagttctaggacagccagggttacacagagagagagaccttg
tctttaaaacacagaacagtcaatcaaaaagcatgtcccactcaagtggctagaactgta
aaacatgcagatggtctgtgctatcagctgggttgttagcagcatctcaaacttacagag
gacttgagtgtcagtcttgtcaactgacacaacactgtgatgtagacttagcatctcagg
ttgtcagatgagggaacagatcgaaacctttattgaataagaatgtgaacaaaactgctt
ccttcttggtactctggctccatttcctgttacatatccaaggccatacctgcaccactg
tgttctattgtatttaaagcccagtgtgcaactgagagccaataaggaagtagccagtag
gtcagaggtcactggggaaagtaaggggctggaaggattctctctcactgtgaatgaggg
agaagtgtgatcattggttaattgtcagctcctgccgctgttttaagccctgtttaaatc
tggaagacatgctctgccttttcttaattacttttggatagagcccatagtggtggagat
gccaagctataacaagaggtttttgttttgttttgaaccttctttgtattctgattttaa
aaagcctgggatggtgggaatcttaacagttggttgtcacccaagtgtggctctggagca
gggcagtttggagctctgaaaatcgtcaatcctgagtcctacttagtgctggttaagaga
caggttatttgtctcacttaagtttagaggatgccacatcccaagtgagaagcaaactgg
aatccagtcgggaacgtcgtcgttttggccttgagcttacaatataatctcacttaatct
tttaaagatgaggtattggatagctttcccactgcagtgcactccagccttccttaaatt
attttaagaatttactatttagtccagtgataatagcaagcatgtcttctcaaatgccat
taagtacactcaccaggatcagatactgaagagacagacaagtggctctctagtggtgcc
cttttcagattatccattctgcttttatggtactggggtagaacatgtgaggtcatctgc
gttgtgcttctgtatcctgctgatttgagaagtggcttgcctcccatagacagtaactgt
ggtgcttgctcctgtcctatgatgtatgacttgcttttgaactcctcctatatatgagct
gaaatttgatactttggtaatagactgatcttggatcttgtgggaccagcatggaattat
tagctgaaagaactccaccataaatactgctttcggtcaactagtagcccaggaggggag
tggtgtctatttctgtgcaagtgactaattgtacgtacacgatggtttgaccgtctcatc
ctgctcttacaaatgtctgtgataaaatggatcaggagtgcaggatccaccacttatgtc
tcctagctcatcagagacagttgtgtgtgataggaagggggctgggagtgagttacatag
gggctgtggagtctaaccatttgctgggcctgtgtgtttgtgtgatggttgcagatgcgt
gtgcactgtggtatggaggtcagaggacaactttaggaattgagctcaggctttcatggt
gagtgttaccacccacagagttcatcttagtagccctgctctgcattcctacatttatca
gattatatgccactttgaagctaaaaatgcacagtcctgaagcaggtgttgggttggctg
cctctaagcccagcactcaggagggaggcaaaagaatgtcaagggcagctcaggtaaata
gtgagaccctgtctcactagctccaggcatgcgggacctcagttgtaagagttgctgcct
cacatgcacaagacactggttcctcagcaccaccaaaaaacaaatgactgggataaggaa
agacagtcttacttcattcaattttgaggaactgttagggaaattgtccaactatggtga
catctgtaaaatgttaaacttgtatgagtttatgtatgcatgccacagcatgcacgtgga
ggccagagaacaccctacaggagccatttctctccttccatagtgtgggtcctagcatgg
gcctcaggccattaggcttggcagctgcctttgattatgttctttgggggcttggaacac
cactcaaatcaaaactactttgataaaaacacttcaaagagtctagtttactctttgttt
ccaggctggcctcaaactcacaagagatctgcctgcctctgcctcctgagagctgggttt
aaaggagtgcaccaccactgcatggctactctttatttttctaaagaaggttttgtgtgt
atgatagagtatgtgtctgtatgcacatgagtgtagtgcccacagaggccagaaaagagc
attaggtcttctggagcaggcagctctgagctgcccaacatggtttctgggaactgaact
caagtcccctcaacagcaggaagcactttcactgataagccatttctccagcctcttaat
ttactcttaaattatgtgtgaagttcaccttttccttaagacaaaatttaggaaaatggc
taaaatctcatttttaaagtcaaaactaggccaagtccctctaatcttcatatagcagtc
agtcctcctgagcctcgtgaggagaggacccctgcctgccctcacttacacatgcagccc
tggtgctctgctatgctctgtcccatgctgctgctgcagtagtagtgagagtttgtgtgg
cttgagtttgagagcctgttgtcctgagtgtactcataccctcatctcactccataaagc
aggacggacactcagcatcacacctgcgggagctctggactgccttgctcctccttcctt
taagggagagcaggatagtactcataagccttttgctagtcatttttgaaaggattccac
tttgaaaatcttaaaacagcctggggtttaatgaggtaaacataggatgatgggattgca
gatacgtgatcctttattatgtgagtacagttaatcatactgcaaaaatgagacaattga
agggcagggggtgtggcttagaggcctgaagcatcaggtttgatctcaagcactgttgtc
ttgctgtgaaaaaacaacttagagaggagagcatttaatttggggcttggttacagcttt
agaggtaggatgctttcatggggtgcaggggctgggcctggttggggttttgaaacctca
cagtttatcccaacgaccccctccaatcttagttatggttaccattgctgtgatgacatg
ccatgacccaaaagtaagttgggaaggaaaggatttcacttataatttcatatataaagc
actgagggcagcgacttggaagccatggaggaatgctgctgcttactggcttgctcttca
tggtttgtttagcctgctttcttacagaacccagaactaccactccaggggagcatgacc
cctgtgggctgggctctccccgatcaatcactaattaagaaaatgccttacatctagatc
atgtggaggcattttctcaactgaggctccctcctctctgatgactcttgcttgtgtcaa
attgacacaaaagtagctaatacacctactactccttctcaaacagttccataactgagg
accaaacaaatctatgaaccatgggggccattctcattcagaccaccacagtggtgcata
gtccatcatactagcactcaggaagtagaggctggaatcagaaagccaaggtcatctttg
actaagtttgaggtgacctgggccacataagacttgccttagaaaagagaagacagagag
aaagaaggcacttacagtgcacgtgctttctgatagcgtccatccaggtgtctcttcacc
tccgtgctttctttacagatcaggaaaaggacatagcaagtaggagtggtataaatgtgg
agaggatagtgcatgtaggaaggcaaggctggtgtggtacttcttaggcagtctttcctg
catcggtatataacatacatacacacacacacacacacaccccttgtgtttccaggtacc
tggtgacccatctcatgggggcggacctgaacaacatcgtgaagtgccagaagctgaccg
acgaccacgttcagtttctcatctaccagatcctccgagggctgaaggtacaggcagtag
ctttgtcatttggggaagtgagttatctctcccgtgtagtaagaacttagaaaagacagt
ttttcttatgaaaaacttttaaaagagttacagaatcaaccaaccctgctgctcttgtat
ttcccaggccagtttactagaaagcaaatattgtccttccctttgatcctttagtctgaa
tatccaagagacttttagccacattgaaaagttagtagcatcctgatacaacctggtatc
cagtcagtgtgatactgaggctttaaaatattcttcggggctatgagctgcttttatatt
ccttctcaggacttgagaggtagaaaaaagaaggaccactgtgtgcagcctgagtgccag
ccaccaaggctgtctggggcggggggacctcttcagaaccacctttgatgtttagcacat
tggagcccttactgaagtcatcctctagaaagaaaataggctgacaactccagtaggtgg
agtgggaattagagtttgtgtaatggcccgctgtgagcgcgcacagtgcttgactgaggg
gagagcactgcaagcactctagaggatatgacaccaaaaagctgaagatggctttcattc
aacctactttcttctcttttgcagtatatacattcggctgacataattcacagggtatgt
attatacctgacgtggtaattattttggtagtgtgggtggggctagaagagtgacatcta
gttcagatctcagccttgtaactgaaatagtgattttctgcaaccttgttctcagtgtag
acctggtaaaggaacacttgccatttgcctgctcaaggcttggagtctgcctttctctct
gagagctcttccacatgtgtgcatctttaggacctgtagggagcagactctgtgcattgt
tgataagctctctctgcttgctttccaggacctaaagcccagcaacctagctgtgaacga
agactgtgagctcaaggtaagaaagaagagatggactgtttgcttctcttaagtgttccc
cagaatcgtgaccaaccacacttaagccacgacttgctatctaaaaagtcatttgtcctt
aggaatatcagactgtgagacttgaactaggctgaacaagctgtttaagtaactttggaa
gtagatgtcggtatcttgttttcatttttaaaagataattaaattagaaatgcaacacat
taattgtaataaagggatattatagtctaaatgtcagttaagccttcctggaacactgat
aattagataggttcctaaaaggaaaaattgcagatctcagaatgatctaaataaaaccta
agtaaattaaaattaagtctttaaaaagtaagtagaggctgccccctgcccagactgacg
agtcagccctgctgtcccgagcgccatgttgggttcttctagcgttgccgccatgaagaa
ggtggttcagcagctccggctggaggccgggctgaaccgcgtgaaggtttccctggcagc
tgcaggcttgaagcagttctgtctgcagaatgctcaacatgaccctctgctgactggagg
gtcttcaagtacaaatcccttcagaccccagaaagtctgctcctttttgtagtcatctat
cttgaggtttctcaaaccacttttcatgaaccagtgactattcaagagagctaagtttga
agtctgtacaaaagcttctctttaacacgtgccataatacactatcttctgctcgtcagt
ccttaacatctacctctctgaattccatgggtttctgtctcacaaggtttaactatttta
tatacactggctgtagcatacaataaagcagcatacaaaaaaaaaaaaaaaaagtaagta
gctgcccaaggagctaaagaggtcagcaaccctataagaggaacaacaatatgtactaac
cagtaccccccagagcttgtgtcgctagctgcatatgtagcagaggatggcctagtcagc
catcagtgggaggagaggcccctggtctttcgaagattctatgccccagtacaggggaat
gccagggccaggaagcgggagtgggtgggttgtggagcagggcagggggaggatataggg
aactttcgggatagcatttgaaatgtaaatgaagaaaatatctaataaaaaagaaaaaga
aaaaaaggaagtagcactatgcttttttatgattttatttatttatttatttatttattt
atttatttatttatttatttatttatttattttggatttgggtttttcaaggcagcagtt
ctctgtgtatccctgactgtcctagaactcactctgtagaccaggctggcctcagaaatc
cacctgcctctgcctcccaagtgctgggattaaaggcgtgcgccaccactgcccagcaat
gaattttttgaaatgtctgcagggggctagagagatggctcatttcccagcagctacatg
gctgctcacagccatctgcatctagagcatctgatgcccactcttcgtctctgtggacac
tgcacagacttggtgcacagatatccatgcaggcaaaacacccatgtacatgaaatggaa
aaggaaaagaaatgtacttctttgtagagtgttgcggccgccagcagctcgcaacatgaa
cggttcgactgagaaggctactcgagctgtaagagaggaatctagacggggcgaaagaag
aaacggagccaagataaattcattctgatcaaagctcaaattttattgttgggacactag
ttataaaagaagggggaggggacccgatttccgccaaataatctctggtccagtaaaaag
gtgcacgtgtgtggctccgcaggttctagcagtgggcgtgacagaacagatgagcgggaa
gctccacccctgagcaagcaggtttcaggctgggggaggagagactacagtagagttgta
atgtccatgtaaccacagtgatgacatagtaacctccatagtttcaaaaggtttgagtgt
cccctcctagtgacccatgactctcacccttcattcaatgcaggcagccactgacccaat
tctgactctggattaatcttgtttcgtataaatggaactgtcgagggtcatgcacattac
tgaattgtgtgctcatctgtattgtgtgctccagcattcccagttgttcagcagtttcct
tttcataagcacaccaaagtttgtacgctgctgtcctattagtgagcacttgtggttcgg
aatataaacctattgtgaacattcctgtgcacacccgtgtgggcatatgtttttatcctt
gagtaaatgggggggggggttgctttgcttgaagctttatttttaaagtttcttggagtg
ttttatcatgtcatgcattctaagtgaaaactggggtgccctctttctgaaagtgacttt
tctgctttcctggttgtgaggtagcagaacttgctgctcccatgcctgacttctgctctt
gcccttagattctggattttgggctggctcggcacactgatgatgagatgacaggctacg
tggctaccaggtggtaccgagccccagagatcatgctgaattggatgcactataaccaga
caggtactgactgcttcagtcgcctccccagatggggaaggggtgctccctaacatggct
ttcaggtgacttagcagctggggccttacttcttacgtcttccgtgtcattctttctgtg
acactacctgaggggagtggtctcttaatcctaacacctatgtttcctgggaggattcac
catttaccagtaacaaagttttgcccctttgtgagttgcttatttcttttatcttaatta
tattctgagttgcacgacctgtggagtcttttcttgagtgacaattttaataagtttgtt
tctatataaaaaagattgtcaaaccttgggtgtcttaaatcctgacttgaatctctcact
catagttagagtcatttgtgtccatgaatatgaatagaaaccatgtcggtgcttgtatcc
tctttactgttccagtggatatttggtccgtgggctgcatcatggctgagctgttgaccg
gaagaacgttgtttcctggtacagaccgtatcctttaacacgttttggccctgtttctca
tgtgtgtgctgtggtagatgtgtgcttccctaactcattggtggggaagactcagttctt
tgcagttccagcgtggcttttgaggtctgtcctcttgttgagttgctcaccttgccaaga
aaaggctttggggttgagaaaacaagcttagaactcacctgtcatatttttgcagcaagt
ccctttattaatgagtttagtctattcctgaaaatccttggtgagtgtacacggccacct
gtattcagaggtatctggttctcacatttgcttatgtactaaacagactgtgggtccctc
tgctgacacgcccgctgagtcctgtcctatttgctgcttaacaaccaacaaaagaagcag
gaagagagaggccctgctgtgtgctcttgttctctacaccttgttgtccctgtttagagc
cagccccactttagtcccatacttagcaccatctgggttttgggcactaagcactcgttc
ctatctgttgggcacttgtcccatggcttaggaataatatcatttaatgtgattgtgaga
acgcccaccagctctgcttaaggagggtgttaggtgaaaatgttaaaacggatgcttaaa
atccatcttcaggaggagttttgtaaaactctagtagcctgcaactggttttggttgggc
ttttagaaataccatagcgacacatgcttcaccttctaactgcagggtgagctcttgcct
aacacattgagactgtaagctcagttgctgtccacttaggctgcccttctccagggactg
ccatgccactgtgtctgtcgtgacagttttctgctagacaatcttttatggagtggaatc
ctctgctccccggataatgctgtctgctcctgtgcgtgctgcttggtgcagctcactctg
ttattctccactccaggatgtcatcacctgaccatctctattcccttgagattttggctt
gcttacagtccgagctcagagggttaggcagggtttatgatgcaccttgtgctgtactct
gggaccatcctgaagctgatctctcttctcttccttttcccctgctgtctcttcagctgt
cctttggctagtcccagagacacatgccttcataggcagacagactaacagaactatcct
tagggaagtgtctcagttcattgcttttgtagtccatgtatgcacagttagatgctaagg
ccttggaatttcgattttagattttagctttttttacttttgaactatgtggctttgatc
aagttttctaatatccacatctctctatttccttaactgtatacagctcagtgattgtga
ctttactgagtccagagccatcaggaagtgctagcttcaccagtggctgtatcagtagct
tgtcatggccacagacagatagatacctgagagaaatacttattttgggctcatgggttc
agaagtctcggtccatgatgagatagagcatcagggtagcaggagcgtgtggcagaggag
gctgctcacttcatggtggccaggaagcagagagggaacgagagaaggttggggagagga
aagcatccaagaacaaagtaccttctaggacacattcccagtggcctgcttcctctccca
gaccccacctcctaatgttcccagcataaaaaaatagcagcgccagctggggtcagacgc
gcagtaggagacatttgaggtccatactgttaatactccatccctaccttctgaaggcca
tatacttagcttgtagtataaaacacactcagtccatgtccaagaatccagatcctcatt
agtctcccgatactcaaggcaaactctcagctgtagcccctgttacagtcaacagattat
atgcttctccttttcagtggcataaacattctcatccaaaggtaaggaagggccagacca
aagcaaggtgagatggagcattaaagtcccatagtttgacatccatcctccaaggcttgg
gtggcatgagctgagttccaaagagcttgggcagcccagctcctgtcaccctgctggatg
gatccatatgccttctctcttggctcactgcctgtaactgtccacagcccacattctggg
gactcactgggacatccccctcacagcttcatggatcacactgtcaggccactcacagtg
attccaaccctggtgtacactgccctgcttatgccaccccataatccttgggttctgcgt
gcctcgaaaaactagcatcgtgctgccaggctgagcagtagctgggtctttttggaccgt
ggcttcagagacctcagagtagctaagcactatgaccaggtcctaaccagccctcagtgg
gcagggggctctgaggtacctgttctctagggtgttttggacgagtttatactttgcatc
tgggcctacagtagttaggggtctctccaatatccagcatatttggtatctttcctattt
tttcagtacaaatttttctgtttgttgttattggtggtagacatttgttttgttttgttt
tgtttgagacaggctttcttcgtagcccacgctcacctggaactattgtgtatttcatga
tagccatagcctcccagtgctcctcttgccttggtctcctgagctaccgtgccttttttt
tatgctgtgttgggtctggatcccagggccttatgcatgctagaaaaccactttaccagc
tgagtcacatcgctaagcagttctgacaggtgttttgagtaaaagtaggagtttacctgt
gtgcgtttagttttaactgggtgaactggaaatcattagtctagtgggcctatccatttt
tcttaaggtcccaaggagttctaaagttctcttcactactctgtcctgtggtttctagaa
ctcttttcaacgcctctaaagaacatacggaccccactccagccagcctagaaggcctga
ggctgtagcctgtgtggtttgtaaatggaacactgatgagacattttgcagactgctctt
tcgtggagttcaattttctccatgggtttctgtttacctcacttttagtcattgtgcctt
tgaactagtgatcttagcttctaagagctgcctaataagactttttgaggcatcatagat
ctgttaggtgtgtgctgtcctgtgtatggctcatagttgaggagcccgtgtggttaatgt
aacttgatgattgattgacttaacctctacaattttaaagtttataacctatcagtatca
attaggttcatggtgttggacagtcgttgccactgatgactgtgctgtgacttagaaaac
ccttccatccagcttaataagagtggttggtgttctagtggttgtgtaaagaacaaagct
cttctctagaagcctggtcggctataagtatcaagtgaattcagctgatagagattaata
gcaaaagagggagcagggagccccagactgtctctccgggtggaacccttacttactttg
attttgaaatgtgtggttagctaggtatggagtcactgatgccagcacgtcagcttcagc
tcaaggaccgtaagctgtgtggccttttctttgctgtatccaatatagacaatacatttt
cataccagagatcgcagacttgacttgttattttcctgtgtaatcaagtataaatttcct
tcacgcataaaagacctttcccccattgtagtggatgtggtactcctaagacgtgaccta
tggtcaggagaggcaactcagcagttaaagagcatagctgctcttccagtctccaggctg
gtgttcagattctagcacctgcaagatagttcatctgtaactccagtctcggggacccag
tgtcttcttttggtctctgtgggcaccaggcacgcacatagtacagataagcacacaggc
aaaacgctcgtacacataaaaataaatgattttttacagatgtgaacagagggtggaggg
cttctggattacactgctgctgctgcactgatctgcgtactgtcctgggtatattagaac
agtctcagagcagttcgtccttcttcaaacatgctacggaacatcacagatagacagtac
cgtgctctagtgaaccctgtgtctgctctgcccagagggtgaagtaaaatgtgggtggtt
gagcaaacgcactggagtgcacagcatatctagctacccattgctctccattgttaagtt
tccggtcttttcctcatttttagttttttctttccttaaactcagggagggcttatgtat
gccaggcagatactgtaccattgagctgcatctctagaagttggtctcagttttgaaaag
aagttggaccgggcgtggtggcgcacgcctttaatcccagcactcgggaggcagaggcag
gcggatttctgagttccaggacagcctgggctacacagagaaaccctgtctcgaaaacca
gaaagaaagaaagggaaaaaaaaagaagagaaaagaagttgcactgctgatactaaagcc
agtgccttagggttggaaagatggctcagataatttctgaattaaaatttggtattatcg
gggctggtgagatggctcagtgggtaagagcacccgactgctcttccaaaggtccggagt
tcaaatcccagcaaccacatggtggctcacaaccatccgtaacaagatctgatgccctct
tctggtgtgtctgaagacagctacagtgtacttacatataataataaataaataaatctt
taaaaaaaaaaaaaaaaaaaaaaaaaaaaaaaaaaaaatttggtattatcatcttacttg
aaatacattttttttaagatttatttattaatatatacactgtagctgtcttcagacact
ctagaagagggagtcagatcttgttgcagatggctgtgagccaccatgtcgttgctggga
tttgaactctggaccttcggaagagcagtcaggtgctcttacccactgagccatctcacc
agcccccttgaaatacattttaaactgatgataaacttacttctttgtggggaaggaaaa
gctatcccagttgtgaaggatagctcactgtggaaatagcaggctgtactaagtcttgct
gtgcactgtcacatagctgggtggcagctttgccttagctcctaaggagggttttcttcc
ataatgtgtttattaccttccaattcccaggtttgctgtagcagctccagccacagccct
cctcgctgtgcacgatgttcattcatcctttttactacttttttttttttttaaataaac
tgcatagaatgtttttctgggttttgaggataggtcagaacagtgagtcaagtgtttgga
tcagtcggtgtttgccaggctggctgcactgggcaaggctggagagatgctgctgccacc
ctcaaaaccagggtagtcagtctgaaccaaccagagtggtggggtgactgggacagaagt
cttcctccggtggaaaagcagaatggaatgaggagtgagaggtgcaggtaagttacttca
gagagaagtccaacatggagtaaaaccccaggtcattatggagaggagacggagactcag
gagtccaggtcacctcagccacatagtaagtttgaggccatctcaaaaaagcttgtccag
aaagacttcttgttagaacagcccttggaagggacagtgggaaaggaaaaggcttaatac
aggaagagtggaggcttcccttacaactgcataaagaggcatagaagtgcctgagacctt
tgccttgagttgaagctttgagcctattcaagaatagtagtttgaggcaagcagataaaa
gttgagtgacaggtgtcagaaaggcgtggatgtgagggtgaggagctggttgtgatttag
tctgagaatgctcacccctgcagacaagggagaaaggtatggatgagccctgcctgctga
tagatgcatgttcttcaggcagcttggaggtgaagctaagtcagttaggaaacaacccag
acttcacttttcccaaagggaatatgaaacttgcctctttagaaagtccagctgggccgg
gcgtggtggcgcacacctttaatcccagcactccggaggcagaggcaggtggatttctga
gttcaaggccagcctggtctacagagtgagtgccaggacagccagggctacacagagaaa
ccctgtctcgaaaacaaacaaacaaaaaaaaaaaagaaagaaagaaagaaagtccagctg
gttaataaactactttggagtcgtacatggtaaaatcacctcttggagggctggaagagt
ctaatttttttctattaacatacatcctcttgaagtggctaatcagatattcctggggag
tagaaatgtgaatggagctagatttatataaactgagtagcttcttcctgtctgcaagga
agatgctcaactgtcaccattagcatgtcggcagtgttacggcctgagtgagtcagtgcc
agtctcagtccacccactgtccgcacctggagcatctcctcttttccattttcagtagca
ggggttttgttttgttttgttttttcttttaccattctaacttcagaaatatcttgtgtg
agtggacttccttctgggttaactcggttttctgtaggatgccatttttgggcactggct
catcttatcttaaaaattattgactgttgcaggcctgtgaaaactggtgtgtaaatacac
gaatgcacggccttgttgcattccaggaaaactccatgtataagacaggcaccaggctgt
atcttcctgtcacctttctagaattttcttctgttttagcaaaaaaaaaaaatgttcaga
ggcaaaatggccatcttgttgcataagcagttcctgtgactaggcaactgtggcttgcag
acactgttctcttgactgactcatgcttattttgcctctctatatgcaagctaaatcttc
tctatctgttctttaaatattgtatgtgccttttatgtatcagataatctaaaataaggg
atggggttcagccatacattgtagtcccagcacttggatagatgaggcacaaggactctg
agttaagaggctgctgggggattttaggtgacacaggaaggacggctaatgatcctgcag
aacctgacgggctcacacagtctccctatctcaccatcagctgaggtatggagcagctgt
tcagatgctcaaaaggggctgactgaaggcagtgagagtaaatggcctctgatcctccgg
tgaacacatgctcatttgttctgaggttttggatggcagaatgactattccgggcccttt
gcctttctgcttttcaagtctttagtgagtagactttggttactttacatccgttatggt
tgcagcctggtgtctcccatagtggggtagcaccctctgccagctggctgtccctcacat
gttctgtctcctagaggggagaggggaaggtctccagagggcacactcaagtgaacgaat
ggtgtctgggaggagagcaggcctcccaactgtcagattacaagttccttaggtcaggcc
ttagggggatagtcacaaatagatgcatagagtgtgctccgcctctgcttgccagcacac
tctgcttagctacctcctagtgccttggtggaaggggaggtggaggtggaggctgggctc
atggactgagtggcgtggcctcttgtctaagacccaaccacagtgactttacttcctgct
gcatggtggtgacaaatgtgtgtgtgtgtggcggttttttgaacctttctacaggaggag
caagtcatgctgaaagtggccaccctacagaagccttccacccagaatcttccaccatgt
tggccttgtttgggggagggggggactcatttccattattgagattttttttgtttgttg
tgttttattttgctcattattgttttgttttgcttttcacttttattttgtcccgacatt
ttcctttatagtccaccgttagatattgatcagttgaagctcattttaagactcgttgga
accccaggggctgagcttctgaagaaaatctcctcagagtctgtgagttgacgctttgat
tgaattactaccctcgggggcctctgccacttgccttccgtcactctgcactctgacttt
ggaatgcatgtgtgacgtgtgctgtgtgtgatgtgtgcacacatgtgagcctgcatgagc
ccgcttgcatgcatgagtgtgttttattctaaactatccttatctgggtgtggacttaag
atcatgggaacccctttggggcaattgtatgctaataaaactgagagttgagattccagt
tcattccaagagattaatgggaaatttgtgtgtgtgtcctcctgagccctgacttctctc
tgaagttgcccacgtgcttaagagccctctcactttctgctgagcatcctaatctctgac
actgtgtgctagtctgaccttccctctcagtggtgggtgatgtgtgccttgtccctagag
gggctcctcctgggcggggtctttccatgggctcctctcccaccctcagaccacacagaa
cagcttcctcccctcatgacagtctgcagatggctctgaactcctccaggaaccttggtc
aatacagtgaatagcatgaggggccacttggaacttgtgctttctccatggaggccataa
gaaggtgactctgttttactttagccatggttggttggttttttgttgttgttgttggtt
ttttgttttttgttgttgttgtttttgttttttggtttttttttgttttttttgtttttt
ttttttgtttttttgagacagggtttctctatgtagccctggttgtcctggaactcactt
tgtagaccaggctggcctcgaactcagaaatccccctgcctctgcctcccaagtgctggg
attgaaggcttgtgccaccattgcccagccttgttgttgttgtttttgttgttgttggag
gattttttttttgtttttgtttttgttttttaagctgttgtcactttctggggccagaaa
ctttgaggaagctaggaaagcccctccttcctctcatttccgcttagggaaataaatgga
ggagctaagccccagctgtgcacgcctaggttcccctcctaccacttccaatagaagagc
cttctgctcagctaagaaggaagttaagtagctgtaggcaaccagcaacacacgagtcac
ctcccctgacaaaatcaggaccccgaatctgcagtgtagctatacataaaggaccctgga
gcttttgaatgggcctattttattttatttcctttactcctttaagataagaaagcaagc
caggcatggtggcgtacgcctttaatcccagcacttgggaggcagaggcaggcggatttc
tgagttcgaggacagcctgggctatacaaagaaagcctgtctcgtcaaaaaggaaaaaga
gagagagagagagagagagagagagagagagagagagagcaagcaagcaagctggataca
tggcctaataatgagatgtcttttatcttaacatgaagtgggcacatcagatatttgaac
agttctagtgcagcaatggacagaatcaggtaccaagacaaacatcctctcagtgataac
tcagttgtgtacagtagtgagaaaagagcttccctgcataatcgtcagaaatgggtaatg
gcagtctaaatgttggaaaagtatgggcaaggttaagttagtgttgatgacaccacccag
tacttttgaaatctccaaagcaccagctgtgatggtgcaggtctctaaccccagcactta
aaaggcagaggcagaaagcctgggcttaatttgtaagacacctttttaaataaacaaaca
actattgtagatgtagctcagtgacgtcaacatggccatgggtctaaccccagaccccct
gggggtggggagcatacagtacctacatccgggtgagataaagctgactgagtaaaagta
gtcagaaaaatttactaaagaattgtagtcaacagaacactgaataactcattccttctg
gaaagaacagaacagggaagccagtgtagacaaggacacggggtctgaatacagagaatg
caggacagggagaacaggacagaaagctggcagaggcccgaggacagctcactgcttagt
gtgctggcacctgggttaaccagccaaggaaacaaaggcatggctcctgccctcttcttc
agagatacatcttcttgactcccagatgacaaatggctgatcctgtacgtgctgcccgca
aggagagcatgacagttgagcgtgtgagctcacctgaatcctgcgtgtctagttcaccct
gtttgtcccctctctcagtatgcagagcattgttgagtgtggagttttcagagggaagtc
caaagtctgtacagagtgagtcagcccatctctgcactcaggtagaagaagggcaaatgc
atagcagtagtcaccttccagcagggttcgtgctgatgtgcaaggaaaggacgccattgg
cttgtctattaaagatgagattccagctgaaggatgacttgatacttaggtgaggagaaa
ggaagctcactaaatcatgttaaggtttagtttaaaatactatcatcaccttgagaggga
gcgtgggataaactggcagtgggtttccatttcagaagctgtggcctgtacctggtgaat
tggtggcctggctgagggaagttagtagtcctgtcgtttgagtaaggacactgagatggc
ttagcagcagagacctgagttctctcctagggcccacacagtgggcagagaattctacaa
cttggagatcaatcctctgatctccattcattagtggtacgtgtgacctgcccctcccta
ccacacaaatgaacgtgaaaatggaataacggaaacttcatgtaactttgccccgtcaga
taaacacagtgttttaaactttttgttcattgaagttagtgtcttccctcactacaggtg
tgattgtggcatgttctgaagctgtcaaccagactgtagcttcggttttgccttcgccat
cactcctaaagtacccttcttcaccaacctatatatttgtggcatgcattttaagtatat
cactgccaacgtctttgagacttggtgtagctcagcacccacactccaagtttctcagcc
tgtaggggtgtgtctcgtagagcctgtgccatcaacactgaacctcctccccagatagat
ctgttagatgtgttaggtttccagagtttcatcaccattatctccatcttctacttttgc
cactgcagcaaacaggttttagccattgggacttgaaaatggttctctggctggcatcct
ggatcaaggcacagtctgcttccagagaacctaaactatttaacattaaagttgatactt
aagcaaaagtttgttttcatgtgtgtgtggccactcactggctacatgcctaatagctcc
tgccattctccacagaatgttacaacacgcagccttaggcgctggcctggtgtggttggg
agttgctttgcttgggatggcttttgacttaaacaactggagttgatttttacaccgtgt
cttaacaatgcccttgactaggcatcttaagatattaaccagcttcagcagataatgcgt
ctgacggggacaccccctgcttatctcattaacaggatgccaagccatgaggtgagaaca
aacagcatgcacagggaagtctacctcggaggccaccttctcgtggtagtgtctgtgtat
agccagcagtttctaatgtcaccgaatgcttgcatgtgccccaagaaccgttaaagcagt
actggctgtgtgctagcggagtgttggcatttaggatgcagtctcctgagcctgcgaggc
agcgatgcagtgtagggcagtgttccctagtgtttggctttctgatcttgtgcttgaggt
aacaagtgtcgttgcagttgtatgtagttagggtgtgctacagccgtgtcatgggtgcat
ggaacagagttcattagtgtgctttgctctccacccattttacaaccaagagaagactgc
atgcaagcacgcactataaaattccttgtgctaataggtgtgctgggttttttttaaaca
agacaagtaattcatagtaagtatgaacacctggggaatcgagctcttgaccaggtcata
actaacctaactccagtttatgatcagacgtgacagacaaacccttgtagagcattgtgt
gcatttcagctcttgtctgtattgaaaaatggcttttcttacctctattcctttttccat
tctacgtggttaagtaggaagggattgcagccatgtctgtgtctggttcctacaacttca
aattgcagtgagttcgtctgcttcagtaccaactcttcttcatgacttacctgattagga
aaatggtcctggaaaagactgcttggtctcaattctggttttaagcttctgaccctttga
ccagaaagctagtaaagagtagctgattgattatcactctcatccacaggaaacagaact
ctggggagaccgcaagaataaagtcagtggtcacaaatagagggggtcagtggctagaag
aagagtaagcctgaattgagcatcccagacagtggtccatacgggcctcagcctagctca
ttccctgagatcactaacactactgaacatagtcattctgaaagtctgtgtttttacagg
caagaaactacattcagtctctggcccagatgccgaagatgaacttcgcaaatgtattta
ttggtgccaatcccctgggtgagttgatcgtactttgattttgtggtataaattagaaat
ttgaagttttggttttgactaaaaagagtaccttttttttttaagatttatttattatta
tacataagttcactgtagctgacttcaaacacaccaaaagagggcgtcagatctcactac
agatggttgtgagccaccatgtggttgctgggatttgaactccggacctttggaagagca
gcccttacccgctgagccatctcaccagcccaaaagagtactatctttaagtaacttttg
ggtcttaataaatatagctgttactgtctgtctgtattgtcacataaggaaatatcatac
agtgataggcttgtatgcttatatgcttgcagaatatggtggagaggctaagggaagtga
ggaatggggaacagggaattatggggacttgggggagaaggaaggaggtgtgaaccttac
agctgctagtggtaggtggagcccagctgaggacagaaaacaggcacagacacatggtta
atgcccagcagtgggtcctgatgtcctctacactcacagactcgtccccctttcctcccc
tctctgcccttcagggtgtaagcagctgcctttgcagcattgatttggtggctggctcct
tgccttcctgcctccattacctccaggatcacctggcagaggaagtgacagtggtagctc
agattgtgtcagcacaggacagtgtgctaaggttctaattcctctctctggtgtagtagg
cagtagtagatacagagccatcagaccaccaccgtctcctgccatgagaagggaacaagc
aggtggtggctgctgtcccctgcctctcccattcctgcagatgttactgtaaagtcactg
cacttaagtcttcgtgggtcatgtccctttctgagggttctagaagccatgtgccagagt
tacttatcctttgaagggctgatgtctgtggcagtgatttcaaaagctgggagcggaagc
tttcacgaactctgtccatagccacaatactgaggagtggctgtggctgtggcaatgatt
cctgtagcaggagaggtgtgtgcattcttggaggagggggtccagcctatgctacaccat
tcaggcctgaggtgtacatggtactgacaggttgggttaggtttgtggcttccctgtcag
gctcactgggctctctgtaacttaccttgagctattgcatcagataagggtctggggtct
ctgtgatctctaatctttccatgttgattccacccctgaatcctcaccagcagatgtgcg
gtacacagtgtctgagtcttctctgatggagctggtagagatccccatttagacactgag
aagagctgccttacaggagtgaatccaggctgctggagactaaaaggaggctctggccca
tctggtgctcttcacagcctgctcacatccaaacgtgcctggcagcccattgccaagtgc
tggtcagaaggaacatgctgttaccgattgctagcctagccgtccccaacaggctgcaca
ccagcgatataaatattaacagccctgttgtcggtcacaggcttcgcatcactgctagcc
tcaagacacagctgagaacttcacattgccctttgatcttagagtgtctatttatgtctg
cctccatacttgaaaagtcttgttcttacttctgaccttgttcctcatctaagtgctttg
atcctgaggacgtggacctctttttaccccagggtagcaggcacccactcattcatgttc
agtataaagtcagcggatttgtagaacgtgaggatttgtatccaggaagtccaccagtgc
ttcagtgttgaagacagagtcatgtggcctgttgtgccaccatgctcactgtctgcagtg
ctaggtttagatccaaggccttatgcacactttgcaaactttactggctatatcccagcc
ctcctccagcactctaaaactggagagccagcgtatgcatgtttgctgtttcctctgtac
cctcaaagcctgcgtttctgtggataactggtgttcaggctacagggtacataatcactc
agaggagcagaggtatagcatctcttcaggaagttctcctcgacacccatggagccttcc
acgctgaagaaatcatctggagctgtagagatggccaacggggcccttggtctgagtccc
tgcaccaacaggatgcctcacaactgtctttaattacacttacagaggatccagcgccct
ctgctggcttttagattttcagccagcaggcatgcactgtagtggagacatacttaaaac
ttaaatctagctgggcgtggtggcgcacgcctttagtcccagcactcaggaggcagaggc
aggcggatttctgagttcgaggccagcctggtctacagagtgagttccaagacagccagg
gctacacagggaaacactgtctcgaaaaacaaacagaaaaaaaaaccttaaatctaaaaa
cttgtggagtttatttctgttttacttttgggagagaaaaaacctggaagccattattga
aagtaactgataatcaccaggagacaaaccatctccccatattattcacttggggagaaa
cggggaagagctcaagatgccaaccaccttagctcagcataaatggagctcctggctgtg
gtgccccaggtcacagcatcacctgtggcatggtctcacccagtatgcctaatttcactc
ttaccaacaattgaggtggtaaatgtaaagactagagagaaatgggagagagtgcacaca
gcataaggagaggggaaggaagcctccatggtggtgtgccacacctgcagtgtccacaaa
tgaaaaccaaggtggatggagaaggagagctggggggtaggagggggtgtttgtttatta
ttctgggtttcaaacccagaacctcagtaatgtttttaaaatgtgccataggcattcaat
gcgtacatggagggaaggggttacatgcagaagcagacaccttaaaggatgctttgtgtg
gatgtgaaaggtgcttggaacttgcacagtggtggcactgatgtcatgtgttttcctctt
ttctggtgtgacagctgtcgacctactggagaagatgctcgttttggactcagataagag
gatcacagcagcccaagctcttgcgcatgcctactttgctcagtaccacgaccctgatga
tgagcctgttgctgacccttatgaccagtcctttgaaagcagggaccttctcatagatga
gtggaagagtgagtccagggcaggctagcgtgggctttgagcttgctttcattttattca
tgttttgtttgttttctgagccagggtttttctctgtgtagccactcagaggaactcact
ttgtagatcaggctagccttaaactcagagatccatctgtctgtgccttccgagtgctga
gattaaaagcatgtgctaccaagtcccgtttatgtaatattttttaacagtgctggagat
tggacctaggactcacacatatttccaacatacctatgatcccagcacttgggaatcaga
ttaaaagtttaaggttaaaaaaaaaaacagactgaagccgggcattggtgacgcacgcct
ttaattccagcactggggaggcagaggcaggtagatttatgagttcgagaccagcctggt
ctacagagtgagttccaggatagccagggctacatagagaaaccctgtctcaaaacaaac
aaacaaacaaaaaaaaaccaggctgtaaagttattcacagctgtgtaggtagtttgaggc
tatcctgggctacatgagaacctgtctgaataaaaccctcgtggatggttgccagcagct
ttctgagctcactgccagggtctctgatgcgtatgttccctgatgggctgcagacacaga
gtgcagagccttcaaactgacagagctttgaggtgcttggcggttttacgtttacagcat
cttttcaggataaagagggtctggcgtatttcagcacagaactaggacaggagtttttta
tagcttacttttggcctagttagggactgccaggaatggagccacatctccacccccagt
catactccttcataaagcacaaggtcagttctgcagttaggactgcctcgttgtgtgcag
agtgcagtgctgctccagggagcactcagaaggagcaccgtgcatgatggagtggggaga
cagttctgtctgtgtccttgaactggagctgctctgctgcagggcagggcaggagcaagg
caggtgatgtagttcatcagaccactgccaggagggaagggctgagtgtctgcctgccgg
agttgtacgcctcacccttccttccttctttccttgccctaggcctgacctatgatgaag
tcatcagctttgtgccaccaccccttgaccaagaagaaatggagtcctgagcacctggtt
tctgttctgtctatctcacttcactgtgaggggaagaccttctcatgggaactctccaaa
taccattcaagtgcctcttgttgaaagattccttcatggtggaagggggtgcatgtatgt
gttagtgtttgtgtgtgtgtgtgtgtctgtctgttcgtctgtccacctatctttgtggaa
gtcactgtgatggtagtgactttatgagttgtgaatggtccttggcagtctgcctgcttt
ctcagagtctgggcaggccgatgggaactgtcatctccttagggatgtgtgtgttcagtg
caaagtaagaaatatgaaaatatccctgttcttagttaccttgccactttggcttctcct
gtggccctgcctttaccatatcagtgacagagagaggctgcttcaggtctgaggctatcc
ctcagccatgcataaagtccaagagaaccaactggctcctggtctctagcctgtgaccgg
cttgcttaatgtcctcagaacctgacaggtatgttcaaaactgtcagtctgtttgtgcct
taaaagggtgagaagggcgcgtagatagttacagagtctcagctgctgacgttctgagcc
aggcaagtgcacggggctgttggatggccagtggggagctggaaaaaacaaggcagcctt
taggaaggccatggtgcatgtgtgtgcatgcgtgtatgtgcagccgccctccctcacttc
aggagcaagctgtttgctgtgcttacccttcacctcagtgcagaggtctccagtgccgag
cacaggcacctgccatcagtagttcctgtgtcatcttcacatctagcagagcacggatgt
gtttgcatgctgtgctcttggagcttgtcctgtcttctggaagccctggacaaggcgtgt
gaaggcttcccagaagttcctgtccacattgcctccgcccaccgacgccatgggcacact
gctccctcctcctcctccagctactttgtgttgaacacaattgattctccaggtgctcat
ggtgcaggaaaacaggacagacagagagcacctgaacccttgccatctgatgtcaccaat
tcaggaaaacgagtcctctcctaggactatccccggttctggaaatcatgttctcctcac
tcatggtgacaagctaagaaagctgaacaaagggagagacgagagcgcctgaagccagga
gctcctttactatctttctcaaaagggttgttagacacaaaccaagtcatcaaggccccg
ctcctctcctcggaagggtcccccaccccccggcagcttgacactgaatccagtgtcaat
ttggggagaaagcagttttgtcttggaattttgtatgttgtaggaatccttagagagtgt
ggttccttctgatggggagaaagggcaaattattttaatattttgtattttcacctttat
aaacatgaatcctcaggggtgaagaactgtttgcataattttctgaattttgagcacttt
gtgctatataaggacccatatttaagctttgtgtgcagtaagaaagtgtaaagccaattc
cagtgttggacgtgacaggtcttgtgtttaggtcaaggtgtctcctctcagtgcagggac
atgcctgctctgtggggcaggcgaggaccctgaatcatttggagcccagaaggaggcaga
ctggccaggtctcaccacctcagtgtgcagttcaactccatgccatcccatcaagatggg
ttagtagcagtgtctgtttttgaatgccaagtgtgatttccaacaattctgctctggtta
tttcattgaagacatctttgcacatgtgaccatgctgtgttaggggctgtgttccaggga
ctggactcgaagctagaactggcagaagagttctggcatccacagcgcaatgctgccacc
acccagtttcttcatcagaagacaagggaacgagaaaactgctgttcgtttgtatttgtg
aacttggctgtaatctggtatgccataggatgtcagataataccactggttaaaataaag
cctagttttcaaattcagttggtttctcaggtttattctgttcctcttcttggttgtggg
acttttgttgtcagctgtgacctagcacatgtctgacagaccctttctaaagctgcttag
gtggacctgaggggaaggcaggtctttgcagtgagtgagttactgtgcagagagaaccgc
atggggagagtggcctagtgtgcgtccacacgcactgttcttgtacagtctgaaatgaat
gtttttcacatcttcaagtaattggtttgatcagagaatatccacagagtttgttacgta
tcttctgtggctactttcctgataccatgacaattgaaatccgaggggcgaagactgcag
ggctatccaggtagctgcttaatgtttagcaaaagttgctttctgactaaatactctaaa
actttatacaccatgtatttttatctttaagttgccaaagagaaggctaggttgtgtgcc
tgtgagtgcaggatgggaggacatctgtttgactctgctgtcttcatgaccctaaaattg
ttcttcagaactagtgagctgtatcatgcatgagaaggctgcagggcctcacagcaggtt
ctcaggctttgatgacac

### Protein Phosphatase—PP1

A phosphatase is an enzyme that uses water to cleave a phosphoric acid monoester into a phosphate ion and an alcohol. Because a phosphatase enzyme catalyzes the hydrolysis of its substrate, it is a subcategory of hydrolases. Phosphatase enzymes are essential to many biological functions, because phosphorylation (e.g. by protein kinases) and dephosphorylation (by phosphatases) serve diverse roles in cellular regulation and signaling. Whereas phosphatases remove phosphate groups from molecules, kinases catalyze the transfer of phosphate groups to molecules from ATP. Together, kinases and phosphatases direct a form of post-translational modification that is essential to the cell's regulatory network. Phosphatase enzymes are not to be confused with phosphorylase enzymes, which catalyze the transfer of a phosphate group from hydrogen phosphate to an acceptor. Due to their prevalence in cellular regulation, phosphatases are an area of interest for pharmaceutical research.

Within the larger class of phosphatase, there are approximately 104 distinct enzyme families. Phosphatases are classified by substrate specificity and sequence homology in catalytic domains. Despite their classification into over one hundred families, all phosphatases still catalyze the same general hydrolysis reaction.

Protein phosphatase 1 (PP1) belongs to a certain class of phosphatases known as protein serine/threonine phosphatases. This type of phosphatase includes metal-dependent protein phosphatases (PPMs) and aspartate-based phosphatases. PP1 has been found to be important in the control of glycogen metabolism, muscle contraction, cell progression, neuronal activities, splicing of RNA, mitosis, cell division, apoptosis, protein synthesis, and regulation of membrane receptors and channels.

In certain embodiments, PP1 comprises the following amino acid sequence (NCBI Accession No: AAA36508.1 (SEQ ID NO: 6), incorporated herein by reference in its entirety):

| | |
|---|---|
| 1 | MSDSEKLNLD SIIGRLLEVQ GSRPGKNVQL TENEIRGLCL KSREIFLSQP ILLELEAPLK |
| 61 | ICGDIHGQYY DLLRLFEYGG FPPESNYLFL GDYVDRGKQS LETICLLLAY KIKYPENFFL |
| 121 | LRGNHECASI NRIYGFYDEC KRRYNIKLWK TFTDCFNCLP IAAIVDEKIF CCHGGLSPDL |
| 181 | QSMEQIRRIM RPTDVPDQGL LCDLLWSDPD KDVQGWGEND RGVSFTFGAE VVAKFLHKHD |
| 241 | LDLICRAHQV VEDGYEFFAK RQLVTLFSAP NYCGEFDNAG AMMSVDETLM CSFQILKPAD |
| 301 | KNKGKYGQFS GLNPGGRPIT PPRNSAKAKK |

PP1 is know to comprise multiple subunits. In particular, the nucleotide sequences of human subunits A, B and C and mouse subunits A, B and C are as follows: Human PP1 subunit A (SEO ID NO:7):

aggccggaaggaggctgccggagggcgggaggcaggagcgggccaggagctgctgggctggagcggcggc
gccgccatgtccgacagcgagaagctcaacctggactcgatcatcgggcgcctgctggaaggttggtcgg
ggcgggggaggcccgcgtccgccgcgccctggccccctgcccggcccggaagtggcgcgcgggcgggccc
ccggggccagcttagactgggcggggacgggcagcgaggttccgtggccgcagctgcccccccgcggacg
cgcctgagaggtccggggccagctccgacgcgcggagcagctccccccctctcccgggctgggggcgcac
aggtttttggtggcagccccctgcgcacgacagtcgcctcccgaggcgctgagggtagctgggggcttcc
cctgggaggcttgaggccgcggtcccgggaggctcagcagcaacagctgcctgtgggcaaggaggggccg
aggaggcctaggcgcccccggaggtatccatggggccacccgtgtcgcccaccctcctcctgtcagggtc
cagggtcctgacaccccattgcgccccagtgcagggctcgcggcctggcaagaatgtacagctgacagag
aacgagatccgcggtctgtgcctgaaatcccgggagatttttctgagccagcccattcttctggagctgg
aggcacccctcaagatctgcggtgagcccccggcccaggtccccttgccttcctaaaagtggcactggac
cccacagagttcctggagccctcctcccttgagcgcagtcctggccctgggttcaggtctagaccttgca
tctagcttacccggttccctgggtcacaggtgacatacacggccagtactacgaccttctgcgactattt
gagtatggcggtttccctcccgagagcaactacctctttctgggggactatgtggacaggggcaagcagt
ccttggagaccatctgcctgctgctggcctataagatcaagtaccccgagaacttcttcctgctccgtgg
gaaccacgagtgtgccagcatcaaccgcatctatggtttctacgatgagtgtgagtggtctgagcgtctg
gggctgcccgggtcctgaaccgcactctttgccattcagtgggaccttcacattggcctgatatctgaca
gacacttgatagactgtgcgctccacgaaggcggggcccaggtccgttctcgtgcctgccctgcccctcc
tggcatggggtctggggaatggttgctgactgaagggctgcccggattctagtgctttcccccagggccc
tgcctggtgacactccccctacatttgctactaagcacttaggatgtttgcagatgggctctgctgtcca
gaagctgagataaaccagacctggacttggcccggctcatgggtgggggcatgcgaggagtgccaaccct
gaggctggtccagattggaacttagtttccagtggactcgggtgctttagaattacttctggcaggaggc
cgaggcgggaagatcccttggacccagaagctccaagttacagtgagctatgatcttgccactgcactcc
agcctgggtgacagagcaagaccctgtttctttacttttttgagacggagtttcactcttgttgcccagg
ctagagtgcaatggtgtgatctcggctcaccacaacctccgtctcctgggttcaagcgattctcctgcct
cagcctcccaagtagctgggattacaggcatgcgccaccacgcctggctgatttttttgtattttagtag
agacgaggtttctccatgttggtcaggctggtctcgaacttctgacctcaggtgatccgcccgcctcggc
ctcccaaagtgctgggattacaggtcttagccactgcgcctggccagatcctgtcttctttaattaaaat
aaaaagtaactagaattgctcctggaaggctgacaggtggcaggggtggggggtggtttgcatcggtgcc
tgactctctcctcccagccacggctgaatagcccagggaccctggctgagggtggggtaggcaggcacct
cacagccacctccccctgggcaggcaagagacgctacaacatcaaactgtggaaaaccttcactgactgc
ttcaactgcctgcccatcgcggccatagtggacgaaaagatcttctgctgccacggaggtgagggagggg
atggctggggaggaggagcactggccgcatccctcagcacccggcagctctccttaggtctcatctggga
agtcactccttccaggaagtgtcccccataccccactgtgctgggctgcactcacttccttgatgtgtgt
cctacatggagtgggcttgtctcctttctcccgggagctctgtgctcctgagggcagggatgcctttgtg
cctgtttgggaacttgaaaaagctgaggggaaggggatgacttctgcctgtctaggcgcccaggagacca
gtggcgggaccaggagggtggtgggaaaggtggggttcattggccaaggcttcctggagggaggatgtcc
ttgtttgaggaagtgaccttcccccggccccccaggcctgtccccggacctgcagtctatggagcagatt
cggcggatcatgcggcccacagatgtgcctgaccagggcctgctgtgtgacctgctgtggtctgaccctg
acaaggacgtgcagggctggggcgagaacgaccgtggcgtctcttttacctttggagccgaggtggtggc
caagttcctccacaagcacgacttggacctcatctgccgagcacaccaggtgggctggtggctccggcct
gcggcaggggaaggggagggccgtgcctgcgctcgggactgactgctcccatcctcgcccaggtggtaga
agacggctacgagttctttgccaagcggcagctggtgacacttttctcagctcccaactactgtggcgag
tttgacaatgctggcgccatgatgagtgtggacgagaccctcatgtgctctttccaggtgagtgtgggga
agaaaggagccgctaggccctgtggctcagccccatgagcctgaccaacactgtctcttttagatcctca
agcccgccgacaagaacaaggggaagtacgggcagttcagtggcctgaaccctggaggccgacccatcac
cccaccccgcaattccgccaaagccaagaaatagcccccgcacaccaccctgtgccccagatgatggatt
gattgtacagaaatcatgctgccatgctgggggggggtcaccccgacccctcaggcccacctgtcacggg
gaacatggagccttggtgtatttttcttttctttttttaatgaatcaatagcagcgtccagtcccccagg
gctgcttcctgcctgcacctgcggtgactgtgagcaggatcctggggccgaggctgcagctcagggcac
ggcaggccaggtcgtgggtctccagccgtgcttggcctcagggctggcagggatta
tctggtctcttgaataaaggtcaaagctggattctc
Human PP1 subunit B (SEQ ID NO: 8):
gagactcggcgccgtggacgtggagagagggcgacgcacctgcgcgaagacggcagcgggagaagccgct
ccagtgcggttgcgcggcggcccaggcgtcgaggggagcctcggcggggagcgcaccgcgcgcctgcgcg
gagagctgcgtgacgcggcggcgcgcaagggacgtgcggagtgagtggcgctgcgggtggggccgtcggc
ggcgctggtgagctttgcggagctgggcggtgccgaggaggaggaggtggcggcctgggtctgacgcggc
cctgttcgagggggcctctcttgtttatttatttattttccgtgggtgcctccgagtgtgcgcgcgctct
cgctacccggcggggagggggtggggggagggcccgggaaaagggggagttggagccggggtcgaaacgc
cgcgtgacttgtaggtgagagaacgccgagccgtcgccgcagcctccgccgccgagaagcccttgttccc
gctgctgggaaggagagtctgtgccgacaagatggcggacggggagctgaacgtggacagcctcatcacc
cggctgctggagggtgagtgcgcgcctggccgcgggacagagggaggtcgggcaccgccgccgacccctg
cgtccccgtctgccgccggaacgcgaggggacccctttcccgccccgagacgagtctctgggagcgcggc
gcggcggacgaaccgaggagggggcgaggaggctctgggcgcgggggagcggcctctgggagcgcggtca
ggggagatcgggggagagggggccgttcccgcggaccctcgggggccaggcccgccggccgaaggctgtg
cggagcgggtctgcgggagacagcctttcggaaaggccgccgggctccgcgcgccgtggtgctcgcctcc
ggtgacccgaggatggggagaggatgcctgccactgggctttgtgcgtccgtggaatcactgttcttctc
tgttctctttgtgcattgcccttggctgcctccgattgtcgttccgggagtgaattttacgtatatatat
ataaacctgtccttaacaggactgcttcgtctgctctgcatttttacacaatgctatatagaatggattc
ttcttaaagagaggataaaattagcgggaattaatctaccgtttgttacttggcctaaaaaaattgtaag
acaagatatttgatcaggttgaagatgcacctaagaaatgtgatgtatgtagatgggtttgctgaaggga
ttggcttttgcatttgatttgagcagagcttaatttctgcacaaacttagtgaaaatctatttttaaagc
ctgtcttgtattcgaaaggtatttgtaggtctacgcagcatttgaactttccgggctcaagtgttcttat
tgaatagttttatgttggtgggaaggcaaggggggaaaacgaagaggtggactagccgggagttaaatgt
attgattcattgcaggatattattcaaagttaaatgcagtcttctgccaaacagtccctctaccctaaag
ttttcgtcatcaataattgttttgtgagtgtgtagaaaagcctttaatcctttttaaacaatttagaggc
ctctttgttagccgccgtccagcgattgcatatgtactgtttgctttcaaattaccttaagataattgct
agagtgaagcagtatgtacttatatttggaaaatcatcaaagaatattgtaatgggggaggggtaattcc
tttttctcaaagtatgtgtcggtatctcaaacccaagttctgtgagtacctgttctgtaactaaatactg
tgtatttgctccagatgtgaaagccatccagcagtcttaagactgaataggaagcaaaagttttaccagg
aactcttatttaccttttcctttactatcaagttattcctgggaggccccctggtgcctgcagtcagtgt
tcactacctaagactttgtttcagatgtgaacatcttgctgggtctttgagaaatttgctttgaaaataa
ttgtattttgtaactgttttatgagggaaatacagttaatttaaaaatgtgtatgtcttgtttacatttg
ggaagtagaggcctactcctttttattttttttttatttttattttttgagacggagtctcactctgtcg
cccaggctggagtgcaatggcacggtctcagctcactgcaacctccgcctcccgggttcgagcgattctt
ccgcctcagcctcctgagtagctgggattacaggcaccccgccatcatgcccggctaattttttttgtat
ttttgtagagacggaggtttcaccatcttggcctggctggtcttgaactcctgacctcgtgatccacccg
cttcagcctcccaaagggctgggattacaggcgtgagccatcgcgcccggcatatttttatatattttaa
actttgcttgtaaatttaaatattttaaaaatatgtttgtatctcaatttatttatattttatttcattt
tccatatattttgtaggcataaaacagatttatgttttaaaatgtttgtatccttttaaattagacgcac
ctttatatgattacaggtacttaaacttcacgagtgggttgtgcaaggttactagaaaagacactggggg
aaaaataagtgattccatttgtttttgtgaattcaaattaaatatttggtttgtacaatctgttttcctt
ttttgggggggcggggggggggcgggtaagaattgtagtccagatacgtttctctcttaatttagcaagc
gttaacaagttgacctcctccttgccaggcaccagcaatcctaagataagacacagtctcttcactcaag
gagctcccagtctaaacaagtgtctttcaggctttttcagcctcctctctgtcctatgttcagcacacat
tctatcagtacttcttcatcctacagaagcatatttagtttgacagtgcccccaagggattcttaaatat
tctaggtttttcagctccttcccagatccagacctgttgaaagacttgtccagagcagacttgaattttc
tagtaaataattatttttaatcttcaaagaaaattttgtccacctggattttcagtgatattagatgcta
gttagtgatattttgtaaagctgcctagcaagaataactatggttagtgtttttagtgtcaaatgaacgt
gtaagtaaaaatttggcaagaatttctctgcattaggggaaaacattcagcaggacctgatctcatttta
aaatgctgattgtagccccacaacatattcctgattgttttttcttttttactgtattaaatcgcagatc
agctatacacacttctggtgactacattttatgagttgctttttgtttttcttttttaggtgggcgtatt
attgataagttttttttgttttttgtttttttgagacggagttttgcgcttgtcacccaggctggagtgc
ggtggggtgatgttggctcactgcaacctctgcccctgcaggtccaagcgattattctgcctcagcttcc
caagtagctgggactacagcatgccaccacgccccggctaattttgtatttttagtagtttcaccatgtt
ggcctggctggtctcgaactttccatcctcaagtgatccacccgcctcggcctcccaaagtgctgggatt
acaggcatgtgccaccgcgcctgaccttgatatgatattttcaaagagtcctttaggtcattttaataca
aacatgccatttaatgtaaaacagaaatattgattttattatttggacggtgcatattctttcggtatgt
tttcttggattaacttttgcttagaactaaaaaccaaagacttctactttatcccctgtagtgaccctgt
tggccacattcttgtggcttaccatagtgttttgatcatttaaattgaagctagtttgtatgagtattct
ggaaaatccggtaaaagcttcttacttagatgagtggttctcaaagaggggtgggaaatgggagtatagc
agaatcatctggggaacttcattcaaactataagtgctaagtttattttggaggaagtgaagtggaatga
gtagaatctaatgcatttgggaggaggttagtatggaaaactgctgctgggtgaataagagtgccagctc
tgttcccaaccacagtcttgatccaggtatttaaaaactgggatgtgccagttgatatagtatgtctatt
atgtgtcatatatgtgataatatgaaactgtttagaatttgctttttcctgattatgaaagggcagcatt
ctttcaagaagtcccctgtaattccacaatccagagataaccgttgttgaaattttgatatatttttgtt
taaagatacttaacatactttgtaaaattcacatgctttacatcattagaatattctttacatagcttta
tgagttaatttttcatgtagtattacatcatgaacattttcccttgtcattgaaatgtcttcaaaaatac
cgaaaagcgatttgctgtgtgatagaaaagtttttcttaaagtattttttaacatattaccacgaagctg
aacttgactattacaatatgagttgtagaatattagcactgtgtaaagttccatagcaaaatgtgattgt
gttacatttttggcccacttacagttttatgaaaatcgtgggatgtgtttgtacctgtttttgtttttaa
attcatttttcatttaactttgacctgtcagatgtctgaatttgtgacactgcttgtgatctcttagtgc
gtatctcccttttctttcttgtgaaacaggtatagtactcttaagatttagttaggctgacagactacac
ttttaacttttcctattcaagacaggctggaatgcagtggcttgatcatggctcactgcagcctcgactt
cctggtctcaatcgatccttctgcctcaacctccctagtggatgggaccacaggcacatgacaccttggc
tggctaattttttttattttggtagagacggggtctccctatgttggttgcccagtctggtcttgaaccc
ttgtgctcaagcaatcctccagccttggcttcccaaagtgttgggattacaggtgcgaggtgctgcgccc
agcctgtagtgtccttttgaaggttaaaaaaaatgaatggatttgaaaaatgaaatcgattaatgaaagc
atattaaagccctccattttaaaatgacagctttatccagatataattcacataccatacagtttgccta
tttagaggatatgatttttagtatactatagagttgtgcagccatcaccacagtgaactgtataatattt
tcaaacacatataccccccaaaaaaccctgttcccggtagcagacaccccatttctctccattccctccc
ggcccccactaatctacttttttttgtctcttagatttgcctattctggacgtttcatgcaagtgaaatc
atacaatatgttgtcttttgtgactggtttcttttaatgtttttaacattcatctttgttgtagtatgta
tcagtactttattccttttatggctgaataatattccattgtatgtatacaccacatttttaattcattc
atcagttgatggacatttttgtttctttcggctattgtgaatacttgtagtgctgctatcaacatttgtg
tagaagtttttttttgtgggcatatactttcaattccgttgggtatatagctgggagtgagatttctagg
tcatatggtagccctatgtttaactttttgatgagctgccaggttgtttaaggcctctttactgattaaa
ttgtttaattctgacccagcaacctaaaggttaggtggttggaaagaagtgtcagttgttgccgacaacc
tccacttctgtagggaaggtgttttgtgtggatgtgcagctagacttgttagaaaaaagagcatgttggg
gagtgggggagatcacccgaggaatattttatctccttgattcttctggatgagatactagaggagacat
aaggtcgtggcatattttttaactgattcagagattaggtgtttgttttctctgaattaagttcagcaag
taagggaaactgacagtttgaagaagtagcccagaggtgtttttattttgctatctgaatgcagacctgg
agcttttctggtaaatatctaaatccaagatgaagattcgttgggatttaatacattatttaaattgagt
ctagagacaaagccacattaagagtcaggattagtggggaagatagttgcatctagtaggtctttttctt
ccttattctccacgtttattacttttaaaagagtatacattccaatgccatgcatttttaatttttttta
attaattttttttttttttggaggcagggtctggctctgtcacccggttggagtgcagtggcatgaacat
ggctcacctgcagcctcaaccttctgggctcaagtgatccttctacctcagcctcacaagtagctgggac
cacaggcatgcaccactaattaaaaaaaattggtagacacagaatcttactttgttgcccaggccagtct
cagagttctgggattacaggcacccagccccattttcaaacatttctaacaggtaaagtaatacaggtat
gaaggaatgttaagattcttagaaatactgacttaaattctgttatgtatttctgaaaggttattctcga
atgttggtggcatatagggaggcaaggggaagaaatagtttgtaaatgtcagtaggtttagcagtggtgt
ggtagatggggattcccagtggagtcttctaggatttgtgattggatacagattgatacccttaggcagc
agccactgcttctagcagctttaaatagccatttcaaggcttattcaagtataaataaccagtagccagt
tttttcctgatttgatagaggaagtcagtccttggtctagatgggtacttgtcatacagtaggaaaaagg
gcgggggcagttgtcctttattctgcctctgcactattcacatgtggagcacagtgatttgtgagagtat
gtataaccttcgtgaacatcactggcctcagaaccctcccttgccaaatttacctgcatctgtgcaaaga
caagtggagatattgatgatatgtgcatatcccagagggaatagcgtttgctttttgatgcatgcccacc
cttgcataattgggaggttttaacatagcatatttcaccataaaggaattttattctaagaatgttaatc
agaatatccctaagtaacagaagttcccatctggtcagttgcaaggtgaccaaaacactttcttggtttg
agtatagtcatatgttctgtcaaatgatgttttggtcaacagtggacttcatgtaaggtagttagtggtc
ctgtaagattagaatggaactgagaaattcctgtcacttagtgacatcataggcataatgtcatagtgca
atgcattactcttgtttgtggtagtgctggtgtaaacctattgcactgccagttgtgtaaaagtacaaca
ggccacgcgtggtggctcatgcctgtagtcccagcactttgggaggccaaggcaggtggaccacttgagg
tcaggagttcgagaccagcctggccaacatggtgaaaccccatctctactaaaaatacaaaaattagctg
ggcttggtggcatgcatatgcctgtaatcccagttactcaggaggctgaggcaggagaatcacttgaacc
tgggaggtggaggttgcagtgagcaaagattgcaccactgctctccagcctgggcgaaagagtgagactg
catctccaaaaaaaaaaaaaaaaaaaaaaaaaagtataacacaattatatagagtacataatacttgata
atggtaataaacagctatgttatatatttaccttttttttttcttttttttgagacagagtttcgctctt
gttgcccaggctggagtgcagtgacgcgatctcagttcaccacaacctctgcctctcaggttcaagtgat
tctcctgcctcagcctcctgagtagctgggattataggcgtgcaccaccacacctggctaattttttgta
tttttagtagagacggggtttcaccgtgttagccaggatggtattgatctcccgacctcaggtgatccgc
ctgcctcggcctcccaaagtgttgggattacaggcttgagtcactacgcagggccctggaatatattctt
attttaaaaaactagttaaaaccgactcaggcatgtccttcaggtggcattccaggaaaaaaagcttcgt
tatcctgggaggtgatggctccatgcatgttactgcccctgaagaccttccagtgaagatgtggaggcgg
aatatagtgttactgatgatcctgactctgtaggcctaggctaatgtgtgtgtatatggcttagttttta
acaaaagtttaaaaagtgaaaaatatttttaaaaataggaaaaggcttaataggataaggatataaagaa
aatattgtacagctgtatgatatgcttttgttttaagctaatcgttactagaaaattcaaaatgtttaaa
aatttttttaagtttctaaagtaaaaaatgttaagtaagcaaaggttaatttattcttgaagaaagaaaa
atatttttatctttattactgtatatacattgtgccttttctgtgtttagatatgtttagatatacaagc
acattgtgttacagtagcctgcagtattcagtacagtaacatactgtgcatcatgctttgtagcctaaga
gcaatagactataccatctaggtttgtgtaagtccactctgtgatgtttgcacaacaacaaaatcacctc
actgtgcatctctcaggaagtttccctttggtaaatgatgcataactggacttgtctgacaccattttaa
ccttccccctagtcctacctctcattctcaagatttggagaaatcaaaatctaagttttcataataaaag
gttaggtatattgggtgttacatagctttcactaatgtcatatctagcagttgtaatcacgtattttaaa
actttaaatagtaaattaagaaggaatctttataggtacttgtatgtttcctcaatgtggtttgaattga
gcgttttatttaaattactatcaaatacttgagccaaactagcgagacaaaaagaccagaccttttcttt
tttccgagatggagtctcacactgttgtccaggctggagtgcagtggtgcagtctcggctcactgcagcc
tccgcctcctgggttctagcgattctcctgcctcagcctcctgagtagctgggattacaggcgcctgcca
ccatgcccagctaattttctgtatttttagtagtagagatgggatttcactaaatctcgaacacctgacc
ttgtgatttgcccaccttggcctcccaaattgctgggattactggggtgagccaccgcgcccggccgaga
ccagacttctgagggggagaaacttgcaaacctgtcagtgagaaagagtgaggtctgattttagatgaca
gaaaggaatagcttttgtgaatttcggactcccaactactagaattatgtgaatcctgaatgataatcag
atgtttactcagttaagtgtttactcaaatattaataagtattgatacattgggatgccatcttatataa
tgattaacgacacagattctgggtgtatttcagccccagtttcttctgcatatatgatagtaagtgcctt
caaacaattcttgacacattaaaggtcagaaaatgttagccgtcattattgcttagtacaaacctgagct
aggaaatactaacagagagctctttgtgccactgagtcctggaacttccccatactaattttgagaaggc
tttcttgtaagtatgtgacagatactgtttcctccactttctgtttattgcatataaccactattcttgt
atgtaagtacgtataacagtttctttactattcccattattcagcgtttataatctggtttgacatatca
gtgttggtgctgagagggaaaggtatttctaggtaggataaaatggtgaaaagtgatttaacactgttgt
atgcttgcttttggccaggaactatagggcagtgtatcttaaactttgagtcttgagaccctcttacact
caaattctccatatatgtaaagaattgaaaacgagcttttatttatgtgaggtatttaacaatattgcat
tagaagttaaaactgagaatttaaaaatttctgttctcggccgggcatggtagctcacacgtgtaatctc
agcactttgggaggccgaggcgggtggaattgcttgaggccaggagttcaagaccagcctgggcaacatg
gtgaaaccccatctcctaaaaatataaaaaagttagctgggcgtgatgtcacatgcctgtattcccagct
acttgggaggctgaggcacaagaattgcttgaatctgggaggcagaggttgcagtgagctgagattgcgc
cactgcactccagcctgggcgacagagtgagactgtgtctctaaataaataaataaatatctgttcttta
agtagcaacatattacattttagcataaatgttttttatgaagataacttttctaaagcaaaaatgtgag
aagtgccatcattttatgcaaatctaatgtctttttaacagaagacagctgaattcacatctctgttttg
tttcagtcttttgataccttgttttagttgaatatgaagaaagtctgtagtcctacagatacgtagttag
aaaaaggaggagtagtttaatagctgttctagataattgtgggtattctctgatgtttataccaaaaatc
agcaagtggtggttttttaaaggtgaattttaaaagagatgtggaatctgaaattatatccatgaatttt
tatgttctcttacattaaaatccattgatctttcttgtactttaaatggatcttacacatgactttataa
tacgcgttgttcatttggataatattgattcagctgagttatgcagatgtccagatgttgaaacagctta
ttatctaatacagtatccagaaatgacatttgttaatgttacaagatctcatcagaagagtccttaatta
tggagaagctgtcagattcatggtgctgattacaaggtttctagaattttagtttgtaacttgaaagctt
gaattttatcattggcaacaagtactgtcagttgttttccttgaaatgacagggtcactttgttcatgtt
cacgtgtatctgttgaatatctaagtctgaataaccatagtttgtcagttaatactttccagtaaaagtg
tttagtgaaaaaaagtgtctaggtctgcttggaactaaaacagtcacaggagtgctttcctttgcgacaa
ccattgtactttgggatggagcaggagtgctttgtgggcatttccattttatcatgtagattattaaaga
gatgtattcaaagattgaagtttaataaaattgatcatttgtactgctttaccaaagggcatacatgaag
ctgccttttttgtgtttcactgcatgtgtgttgcaatgaacaacacagtgactgactaccagtggggttt
ggtgccactgacttggtgtgtactgagacacttaacagttttacccatccttgcttttgtattatcaaag
cagatgtcagtgcagtggaaaaaaaaaaagtcctttattcctgaaatgtctagccctcacctatatctgt
cacttttcttcttcactcttttctcattatacttgctatttgtttaccaatgtacctgaaatgacttttt
acttatgctttgtgaattcatgaaagccatgggccttctcagaaaaatatttttacatatacacaatatt
acagacaatttgtggtactatacagagccactaaaggccattttggtctctgaatccacgttagttattc
ttttgttttgagacggagtctcactttgtcacccaggctggagtgcagtggcaccatctcagctcactgc
aacctctgcctcccgggttgaagcgattctcctgcctcagcctcccgagtaatctgggattacaggtgcg
cgccaccacacctggcttattttcgtatttttagtagagacggggtttcagcatgttggtcaggctggtc
ttgaactgacctcctgatccgcccaccttagcctcccagaatgctgggattacaggcatgagccaccatg
cccggcccacattaattattgttaacaggcagtcctaagatgagagggaagtacagcagctgcctaattt
tttttcacttaaaaccgtgtgtgtgttgtgtgcatagagaaagatggggaggaaaggaagacagagtggg
agagggagaagaaaagcaagagagaaagaatgaaaaagcaaagggggctggacgtggtggctcacgcctg
taatcccagcactttgggaggccgaggcaggcggatcacgaggtcaggagatcgagaccatcctagctaa
catgctgaaatcccgtctctacttaaaatacaaaaaattagctgggcatagtagcacccacctataatcc
cagctactcgggaggctgaggcaggagaatggcgtgaacccgggaggcagagcttgcagtgagctgagat
tgtgccactgcactcgagcctaggcaacagggcgagactccatctcaaaaaaaaaaaaaaagagagactc
cacattttagccaattgtagaatttttatttttctgcctttttgttgcttaaaagcaggcttttgtgcta
aatatggagaaaatgctaggaaaaaattaggatgtctagatttaactgccattaattagtatgtaatctt
gggggtgggggttgagtaagagttaggataccagtggtcagtgaagattactaagattttatgaggccaa
ggcgagtggatcacttgaggtcaggagttcgagcctgggcacatggcgaaaccccgtctctactaaaaat
acaaaaaattagctgggcatggtggcacacacctgtaatcccacctacccagtagattgaggtaggagat
tcacctgaagccaggaggtggaggtttcagtgagccaagatcatgccattatactccagcctgggtgaca
gagagaccctgtctcaaaaaaaaaaaatttatgagacaccgtgcataggtgttggaattagtgctttttt
cttgccatcttttctgttttagagaggtaatgataagtaagaacttgctggggaaagtagcataaggtta
gtgaaaagtaaaatttagtatacacacaacacacagacacatatatatcacatacccttggatatgtgcg
ttggaaagacaaacctgcaatggaaaagtttttacactgatataaaatgtgaaatataatggttgagtaa
aggacagaattcttttgggagaggatacacattttgcttaattgagattcaaagttaatgttctctgtca
tcaaaactgattgcaagtggtcatctcttgatgctgatctgtagtgagattctatgtatttcatcttaga
agatgtctttttacacagggacatactgcatattcatcacttggaaagcatttatagaattctcctgata
cttgcctttcagcatgtcattacattgcagattaatcacttccagtagagggttaggtggaagctcctcg
gctgcacagttaaatggattttgatatatggaaattttttttgcgcttgagtcaaggtttgaagaatgct
agtttgagtttgaaaaaatgtatctgctgcaaaattgtgtgggaatgaagatctcacttgtcttaacttg
gcagcacgggttatgtataaagcagtttgctgactgggtatggtggctcgcgcctttaatcccagcactt
tgggaggcggaggcaggaggatcacttgagttcagcagtttgagacctgcctgggcaacatagtgagacc
ttgtctttatagaaaattaaattagccaggtgtggtggcatgtgcctatagtcccagctacttgggaggc
tgaggcaggagaatgtcttgagccagtaggctgtagtgagcctcattacaccactgcactctggcctggg
caataaagcaagaccacatctcaaaaagtaaaaaattaaaaaataaagcaacttgctttaattcagacag
tattagttgtcagaatgacttaccacaatggaagtttctcatatatttttttgccttataattttaggct
gaatttattagaaaacatttaggggagctttcattctattttgtagaatagaggctacctcgaattaaaa
ggggaaaaattactagatctgccgcaaaatttaatttctaaaactcttctgtgtttggtaatagtgattg
agggtggtggttcttgtttagaaaaaattcagtatcagccctgaatgcaaataaattacaataaagctgt
cactgctaagccatcaaaatcctgtgtggttggccaaatgaggatattcagcttttattactaacaaata
gtcatgtaactgacagtggttaagttcattagagcaaatgaagttgaccattgatgcctgtggttcagta
atacctgattaagatatttttccacaaaaaaactgcaccagacttcaaacatcctacattttctcagact
ttgtaaaatagtccaactaagtatttttctactttaccatcatcagttgtgacatatcttagcagattac
acttcaggttgtactaaaatagtttttctcagctgctttgagaatattcttgcctctagttgttccacat
agattatttatagagggtaacttttcagtcacttcgaatgaggattggctccccaaataacaagtattgg
taatacctgtcatcgagagccaaggaacaccactcagaaccatttggcttgttttttaattgtttactaa
ttttgctcccagtgttctcaactcttcaagcaccgtttttacagaaaggctaatggcgtttaaaagttta
ttgtctcagctggggtggtggctcacgcctgtaatcccagcactttgggaggccaaggcaggcagatcac
gaggtcaggagattgagatcatcctggctaacatggtgaaaccccgtctctactaaaaatacaaaaaatt
agccgggcgtggtggcgggcacctgtggtcccagctacttgggaggctgaggcaggagaatcgcgtgaac
cagggaggcagagcttgcagtgagccgagatcacaccactgcagtccagcctgggcaacagagcaagact
ccatctcaaaaaaaaaaaaaagtttattgtctcaaaaaatacatagttcttcagcttttccaatcagata
caatttaattaacccaccattgataaatgacttaacttggctaacaaatgggccacttggaaacttgagt
tgcagactcatttttatttttgtgaagaaattctgctgtgatgagatttcattttaattttttaaaaagt
tcccttaccattgctttactatgaattgggaatgctgtggtgagtgctccatctggtaacatcagtatat
gttgtggttttttttttagcacagctacataatgactacaatgccttagcacccaattaaataacataat
ccacactttattgtgtcttaaaaacacaacattcaaagttcactttttttgctttgacatgatgagtatg
cactggtagtaaaagtaaataaaatgttgcagtacagcaataatcgtgacacccatgacgctgccaggtt
gtaactgcatctgtataatttgtgatgcaccgaaaagcagtgtgaagtgatgagagtaccatatgtagtc
catcacaactgctcagctctgccattgtagcatgaatgtgtccagtgataaataagtggctatgcttcat
taaactttatggccaccaaaatttgagtttcatataatttttatatgtcacagaggattcttttgttttg
cttttttgcaatcatttaaaaaatgtaaaaaattattcttaggcccctagccatgcaaacactggcattg
ggctagatttgaccccttggccttagtttgctgaatactggtgtagtttttctgcctttcctaatgtaaa
catgattgtagaaagataaccttactggaagtcaggaaaccttggtgctcccaccaaagttgtgtgactg
attaagtcaggtgaaataaattgggattaggagccatggtgtctagtccttgacaagtcattcctcaatt
tttttaatgtaatcacctgaatttctagtgtatgcataatcacattttgtgtgcctctcggcagccattc
cattgttgaagaggcagccaacacagaaaattgttgaggggttttgatctttgacaagggaagcgtatag
tgtgagctcaccagtcaccctggcttttttccctggagacatttcctagtttgtggcataggggaaatag
atcccaaataaaaagtgacggtctcactgggctaaggaagaaaggtggaagtttggggccactaaagcaa
ttgaagcttaagtccaggagaggagagaaccacataaaatctaacctcaaaatctgcagtcaggtccctc
taaagtcattgactgacttcagaggtacacataagcaagagaagaaacccagcagaaaacagtagctgaa
aagctaaagatctgagcagacatttcagtatctaatgccagagaggcaaaaatggaagttcaaatcctgc
caagttagaggggccttattacaccctgagttttccactgtggccccatcatgaccactgcctaagattg
aaggcaaaactaaaacagaccagccctcaggaaaatgagttactgctgtgctgctgctaaaaggaaacag
ccttctttggagaaaggtattatctaaaacttctacaattttgtatccataattcagtatttcattcacc
aaaataggatatgctgttttggacaagctaatgaaactgaattgaccaaaaaccaatagaaattatctag
tgctgacagaaaaaaaaaagtgtacagaagaaagcatgagagacttatgtattgctgatagacagtggaa
aggtctatcatgtataataggaatcccagaaagagaggagatagacatacaatgggatggaaatattgac
taataattttccaaaacagagatatccaagcacatattaaagaagctgtgtgattccaagcagggtaaat
agagagaaaaccatattgaacatagctgctagaaatccaagagaaagagaaaatgttaaatgctgtcaga
agaaaaaattcattatcttcagggaacaataatatgaatgatggctgatctatcacctgaaactgtggaa
gacagaagacaatagaatggtgttctctttaaagcactgaaagaaaaaaaatgacaacctagaattcttt
atttattttgagatggagttttgctcgtgtggccgaggcttggagtgcaatggcgtgatcttggctaact
gcaacctctgcctcccgggttcaagcgattctcctgcttcagcctcccgagtagctgggattacaggcat
gtgccaccacgcctggctaattttgtatttttagtagaggcagggtttctccatcttggtcaggctggtc
tagaattctaaatctagaatatccttcaatagtaaagtaaaatgaagacaaactaaagctcagaaaatat
attgctgatatgctgacactagagaatattaaagcaagttctttgtgttgcaagaaaataacagataaaa
gcaaagaactgcagaaagaaatgaacaatggtaaggataaatgtgagtaaatacaaattctgactgttta
aaataatagttgtggcttgtgggatttaaaatatgtggaaacaaaaatacgacaactgtagaacaaaagg
ctggaggcgggtggtaatcagtcaaaagtgtatttgtaatctctagagcaacctctaaatgaaaaaagaa
tatataacgaaaagctaatagaggagaaaatggaatagtaaaaaaataactgattactccaaagaaatgc
agggaaagagagaggaatgaagaacagatgggacaaacagaacagatgacagaatggtagacttaaactc
aacttcgtcgatgattatattaaatgtaaaaggagcaaatattggctgggcaccgtggctcacacctgta
atcccagcactttgggaggccaaggcggacaggtcacttgaggtcaggagttcaagaccagcctggccaa
catggtgaaaccctatctctactaaaaatacaaaaattggctgggcgtgggtgcctgtaatcccagctac
tcaggaggctgaggcaggagaatcgcttgaatctgggaggcaaaggtttcagtgagctgagatcatgccg
ctgcactccagcctgggtgacagagtgagactccatctcaaaaagaaaaggagcaaacattgaaattaat
gcaaagctcagaagacctaactattatagatactgtttacaagggatgtattttaaatctaaggacacaa
atgaggaagtaaaaaaaaggggggggggaggggggtgtgggggagacgatatcatgcacacattaatcaa
atgaaagctgacttggctatatttatgttggaaaaagctctaaaaccagaaataacactagaggtaaaga
agggcattatataatgctaaaaggataaatttaatggaaaaatatctaaaatttttatatatgtaatagt
ataggtctaaaactaaaaggaaaggttgacaactattataaaaagttaaaatagacaaaaccacaaccat
agatcagtgcccctctttcagtaactagtagaacaaacaggaaaaaatcagcgaggataaagatttgaac
aacacagccaccctgacctacttggcggacatagaacacttcactcaataatttcaggatatatatgctt
ttcaaaatatgtatgaaacatttactaaaatataccatatgctataccataaagcacccctcagtacatt
tgaaagaaatgacatcacagatgtcagccaaaatccatttaatgaaaaacgtgaaagactgccatactag
agtatataacattgctgagagaaataaataaaaggagagataccatatttgtggattggaagacttgaaa
ttgtgaagataccaattctgtctagacttacctacttattccctgctcccccccccccaccctttttttt
tttttttgaggcagagtttcactcttgttgcccaggctgcagtgcagtggcacgatcgtggctcactgcc
acctctgtctcttgggttcaagcgattctcctgcctcagcctcctgggtagctgggattacaggcacctg
ccatcacgcctggctaatttttgtatttttagtagaggcggagtttcaccatgttggccaggctggtctc
ttcctgacctcaggtgatccacccacctcggcctcccaaagtgctgggattacaggcatgagccactgta
ctcggccttattcccaatttaacacctcacaagtttgggggtggaggggctagacacaagccaattttaa
aacatttgaaagttcaaaaggaaaatctaaagaacaaaattgggatgtttatattactagataccgagac
tttataaaaagccatagtaattaagacattgtggtattgatgcaaaaatagacaaatggggtaatgagct
atatagtggctaaaaacagacctgttcctgtatggtcacctgatttttctacaaagctgccattgcggtt
cagtggggaaaaggatgatctttatgataaatggtgctgaatcaactgggtatctgcttagataaaaacc
ttgacctttacatcaaaccatacacaaaattaatttgagatgggttatagaatgaagtgggctaaaacaa
taaaatatctggaaggaaacataaggaaatatgtttagagaaggcaagaatttcttgaataagatgcaaa
aggccttaaccaaaaaaagattgactaagattttttaagatttaaattcattaaagaacttctgttcatc
aaaagatagcatgaagagatggaaaaagtccagttttaaagaatgaacaaaagacttaaaacgaacttgt
ccaaaaaaaaaaaaaaagataacctaaagggtctattaggcacatgaaaaagatactcaatggccaggtg
cagtggctcacgcctgtaatcccagcactttgggaggccaaggcggggagattatttgaggttaggagtt
cgagatgagcctgagcaacatggtgaaaccctgtctctacaaaaattagctgggcatggtggtgtgcacc
tgtaatcccagctgctcaggaggcaggagaatccttgaacccgggaggtggaggttgcagtgagccatga
tcttgccactacactccagcctgggtgacggagcgagactccatctcaaaaagaaaaaaaagggtacttg
acaaccttggtcctcggggaaatcaaattaaatccaacgaataccaccatatacccaacaaaatgactaa
aattaaaaatactgataatttgcagtatcagtaaagatatggaccaacagtgttaggaggattatgaatt
ggtatagccactttgggaaactgatggtatctactacttaatgaaataaactgacccgagcagttctact
gctttgttctatacatacctaagataaatatgcccatggatattcatagcatttgtaatagccccaaact
ggaaataatccaaatagagtagtaaagatattgtcatatgttcatagaatggaatacaggttgaatattt
tttatccaaaatgcttgggaccccaagtgtttggaatttggatttttttcagatttttgagtatttgtat
tatacttaccagttaagcatccgtaatctgataatctgaaatctgaaatgctccaatgagcattttcttt
gagcatcatattagcactaaaaaaggtttcggttttggagcattttggatttcagattagggatactcag
cctgtatttcaaatgactgttaaacacaattatatggatgtatctcacagatgcaataatgagcaaaaga
gcattcttatgtttctttttatctaaaagtctaattcacaaatacatcaaatcaatatatactgatagca
ataaggatagtaacctcaggcattactgtcattagttagggtacaagggaatcttcagggttgcaggaag
cattctgtaccttgatctggatgatggttactcaggtatatagatagtttaaaattcgttgagttacacc
cataaagttagtatactatatgtatgtaatatgttaaaatattttctaaacagtagtaagtatggtagat
atttgctatcatgggattttatctggtctgaaattactacatgctgaaaatttctaggaaatttttgatc
caaaaattttatgtggtgaagtggaaaaagtactggcctttaagttagaagactaagtctgaatccctct
ctgctgcttctcactcctatcagtcttataaaacctgggcactgtgatcttatgcgagttattgaatctc
tgtcattgtacagtggaaagaatagcttctcttttcagttcctggggtgtgaaaaggattaaatgaaatg
attgatgtgaaggtgctttgcacctaatgaagtatgctgatttataatatttttataatccagcagcgtt
cagatatccaaacactggaatccctcatggatgctgtgatagcagtctagttgccctgtgtaacaggttg
cccggatgatttcttcctttttttcagaagtcttgagagtacataaaggagcaggacttttgaattggaa
gacagctgggattgccctgggacatctgccttattctgtccaacctttggctaatttatttctaatttct
aaaactcttgtcagagttttaaacattgggaaaacctttagcttctctaactgcattcatcaactcttaa
gatgtgactgattattaaatgcacttttatgtgccactaagttggtggggggggctgccaattataattg
taagaagcttttaattgcaagagtctcacaacttcagagatattagttaagggagaaatgtgcatcctac
agttagtgaaaatataaaaagtttctgtgcagtgtgtttttggtaaatttttagtttctgtggatttgct
tgacttacaagttgaagcactttatagttctgtaaaaatgttacacaattacttgagaagtgaaaattat
acagagggaataaaataaatactaggtaaaatattcaccactgtcatttgaatattttaaattgacatat
tgaagtgacccacattagtcttttgacttagtagtcaagttacaactaagttttgataatgtactgttct
tcttgttcattgctgttctctataaacaactaatacacagtagactctcaaacatttgttgaatgagtta
atacagtttgtaagtacttttagctgtgtgtttgttatctgaaagcagtttgagacccttaattcttcca
aggcgaatactttttttttgagacagaatttcgctcttgtctcccaggctggagtgctgtggtgcgatct
cggctcactgcaacctccacctcctgggttcaaggagttctcctgcctcagcctcccaagtagctgagat
tacaggcacccgccaccactcccggctaattcttgtatttttagtaaagacagggtttcaccacgttggc
taggctggtctcaaactcctgacctcaggtgatccacccacctcggcctcccaaagtgctgggattacag
gcatgagccaccgcacctggtcacattttttttttcttaaactaaaatcttccacatttaaaactaaagc
tgagatgtctttttggtgttagaaaaacccaaatatgcatcagaagtaggtaattgatgtttgagaacaa
gcactaatgaagctatatcagttcccataataaaagcagttctgctgatcattgttttgaatttcaggga
tagtcttataaactagcatatttgctaaagcacatttgaatgttgtctgtgtctcctgtttattgcctcc
tttttacttttatattaatatattaatggaatatatagcttttgcatgtggaggcctttttttttttaaa
gcctcttattgacctgacaaaggaatcagcaatttttagcaagaaatatgtggaattttattttactttg
gagatggggtctcactctcacccaggctggagtgcagtggcacgatctcagctcactgtagcctctgcct
tctggggtcaagtgaacgttccacctcaacctctgccttgagtagctgggaccacaagcatgcaccatca
tgcccagctaattttttgtattttgggtagagatggggtttcatcgtgttgcccagggcgatcttgcgct
cctgagctcaagtgatccacccaccttggcctcccaatgtgctgggattacaggtgtgagccaccgtgcc
cggcctttatttttattttgcagagacagggtcttgctctgtcaccaagactgaaatgccctagtgccat
catagctcactgcagcctcaacctcttggcctcaagcaattctctactatgctaggctaattctgttttt
taaaaacatcttttgttgacacagggccttgctttgctgcccaggctggtctcgaactcctggcttcaag
cagtcctcccgctttgaccccctaaagtgctgggattacaggggcaagctactgctcccagcctattttc
ttttcttaaatgctctttcacttaagattaaatgagagctcagctgtaaatttaaggttaaagggagtca
ggggagatggtgatgagaggaaatgattacaagttgagttcagtggggcaaataaggaactcttctgtct
ctccatgcaacattctgttccccttcatcccacaagttactggaaaccatattatcttatggctcagtaa
tgcaaagatgcctcactccatctaaatagcctttagaggttttaatataatgatctattgggaatgtaac
gataagttaatttttaatacatagctgatatcttttttgtatttcttttgatcagaaagtagagatagga
gttgctaaaattttggggactgctggtaacattccaagaaagtgtgtgaagctaaatcacctactgacaa
ttactatttaatctccctttcttcatagcacattgttcttaatgaggctgttttgacatcctactagatg
cgagataagcaggccagaccttaaagggtgcgggctctgaaatgtttttttgtgtgtgttttttttgttg
ttgttttttttctgtttgtttgttttttttttatttgagacggagtctcgctcttgttgcccaggctgga
gtgcaatggggcgatctcagctcactgcaacctctgcccccgggttcaagcgattctcctaactcagcct
cccaattagctgggattacaggcatgcgccaccacacctggctaattttgtatttttggagagatggggt
ttctccatgttgatcaagctggttttgaactcccgacctcaggtgatccacccacctcaacctcccaagg
tgctgggattacaggcatgagccaccgtgcccggccctgaaatcttttaagtctctttggctactaggcc
ccaggttggtcacaggacagcaaaaaacaggactgcatttcatcaacagcagacttgaggagcatcatca
ggaaccagtaacaatgaaaatagaagtcagagatcatagaataacattattttaaaccatgggaccaaat
agggtaatttgctgcctgtgtgacttttctgatttttaaagtatgggcatgactctttttgaaagattat
tatgagtaaattttagaaaactgactgttttatttatcgtttgtcagtacgaggatgtcgtccaggaaag
attgtgcagatgactgaagcagaagttcgaggcttatgtatcaagtctcgggagatctttctcagccagc
ctattcttttggaattggaagcaccgctgaaaatttgtggtatgtaaatgggtaaagttggaaacaactc
ttttgaatcatgtttttcctcccatgtttaagatctagagaaaacattttttgaaatttttgtataaaac
agatcagtaggctttactaaataaaagtgtataaaaattgaaacttataaaaatgacaagttacagacaa
gatgaaaatacttgctatatatcaacagataaaaggagttctatttataatatataaagagttcttataa
ataagagaaaggcgatacagtataaaaataacataggaagaggaaggtcttagaagaaattcaaatggcc
agtaaacataaggacgtcagcttctttttttagtaatcagtgatgtgcaatttaaagcaagtctccaact
ctagtcagattataaaaaaataccaaagattgatgtttagtgttgtcaaggatgtcacgaaaagggcatc
atagtacactggttagaaaaaaataggtgtataatttagagggtcaatatggcgatatctgtggtcttct
aactttgatgttatttaaatttgttttgaagattatactactttataccttgtattcttttaagttttac
agtgacttttttacagtcctttgtacagtgcccagcaaataagagatactcgctaagtaaatgtttgttt
tttgtttgtttggttggtttttctttgagacagagtttcactcttgttgcccaggctggagtacaatggc
acaatcttggctcactgcaacctccacctcccgggctcaagtggttctcctgcctcagactcctgagtag
ctgggattacaggtgtgagccaccacgtctagctgactttttgtatttttagtagagacagggtttctcc
atgttggccaggctggttctgaattcctgacctcaggtgatccacctgtcttggcctcccaaaatgctgg
gattataggcatgagccaccgcagctggccaataaatgtttgttgaatggatcagttaacttagaatatc
acaccttcaagtaagaggtagtagttcagaatttgggggaactaatcaatcccctgtataaacttaagca
aatgtagttacttagaggtgatcttaaaattattagaattaaagtattctttgaaggtgttctttaaaat
gtaatatagaaataacttgaaggtaatggaagttatttgtactacatatttctaaaggtttactatttgt
ctccatatttggtgttaaaaagtgacgtaagttaaactaccagataataaaattgttttagttacctatt
gttgtataataaatcattccaaaactttgtggcttacagcaacattaatttaccatctcacaatttctgt
gggtcaggaattcaggaacagcttagtgggtcctctggctctgggtctctcacaaggctccaattaaggc
gtgatctcgggctgcagtcatctgaaggttgaactgtgaaaggattcagttccttatagactgatggact
aagagcctcagttccaaatgagctgttggtcagaggcctccctcagttctttgctacgtgggatgcttca
ttagacagctggtttcatcagcgagcatggaagaaggtgagagagtgcaagcaagatgaaagtcacagtc
ttttgtaacttaatcttggaagtgacagcccatctcactttcgcttattagaagtaagtcaccaaatata
gcccatactcaagggtaggggaggattacacaggatgtgaacattgggaatcatttcatagaagctgctt
ataacagtaaccaatttttctaaaactgactctttctctttttaggagatattcatggacaatatacaga
tttactgagattatttgaatatggaggtttcccaccagaagccaactatcttttcttaggagattatgtg
gacagaggaaagcagtctttggaaaccatttgtttgctattggcttataaaatcaaatatccagagaact
tctttctcttaagaggaaaccatgagtgtgctagcatcaatcgcatttatggattctatgatgaatgtaa
gtgaaaataaagacttagaaaagtaattcatggaaacattgcctaataattttctggttcagaagagttg
ctttgattcagagttttttcaaaataagatctgtcaggcataggccaggaagaggaacctgaaactggga
aacatgttactaattaaattggttccttgagatctccttggtcatatttcagattgactgcagcttactt
ggctgaagaggctcttttaaaattagtttatctagtaaactatccaggtgaaacagaagtcactgtaggc
agtggcccttggttttctagtgtccattaaggttttaactctcggcattcttgtggtatgacagctttta
ttagtcctgtgtcacaagaattattgagaagttctgtgaaactatttaaaagtcaaagtttaatattatt
agaatttaaatctatgtctgtaaaccttgaaagataaagatactgaatactctggggtatccttatttta
ctctctttttcattagtaggaaatcaacgatttggtattgtatgtttccaagtgttattattaaaatacc
ctactaggttcaaatatggtcttttggttttattagaatacgggagtaagtatttatttggttaattact
acttagaatcaattataatcttcaaattgaattttttattgctgtcttccctattagaatctacttttgc
tttttttcaatccttaattatgttagagacctattcaaactcatatctccttgatttatataatgatttg
acaaagtttgagggtatgttatatgtgcaggcactattgtgaacacagtgcgtgaagcaatacacaatac
agatgatctctgttctcatggagcttacattttttttcccaagtgtttctccagcattgattggccttcc
ctgtccagccaaatagcaggtcatcagcctagctgggactgtatgtccttgtagtgaaaaaaggctgtat
caggcttccaatgcttaatgaaaatagcctttcttttttcttttttcttttcttttctttcttttttttt
ttttttttttttgagacggagtctcgctcttcttgcccaggctggagtgcaatggcacgatctcagctca
ctgcaacctccgcctcccgggttcaagcgattctcctgcctcagcctcccgattagctgggattacaggc
atgtaccacgttccctggctaattttgtatatttagtagtagagatggggtttctctgtgttgatcaggc
tggtcttgaactcccaacctcaggtgatccacccgcctcggcctcccagagtgctgggattacaggtgtg
agccacggcgcccagccatgagcttacattctaatagaaatgattctgtatttgattttgtattgtattt
ttatttctgagatgtagtgtgtggtttgaattatttaaatcagactagcagagagaaagtgggttggagt
acatcaaactacaacattcttcctaaagctacatcagaaatggaggtatatttttaaaggataaaagata
ggcaagaatgtctttggggatagaaatgacatcaaatgaaataatgtatgggagagcaacttgtaaacag
tgctttgagttatgtacttactcattaaggggcaggttgtggcataaaagcacaggaagttagtacttgt
gtctttcatcccttccctagcttagtagttgtaccttattaccaaattcaggaaaatcgagtaactctaa
aaaggtgaacatacacttgaggttgtctttgtcgtagctttttatgctaggctacaggaaaattatagcc
tttgtgcctctatttatacttgctatagtgccaagttgtaattcacacaaaagaatactagttaaacact
tggaaattttccttccttggcaataagtttatactgtgttaatcaatgcattagtgttaaaatggacagt
ttccagaggcaccttttcacttctgcttgcttatttggctaggtatttaactagtccacacactgaacgg
aaaaaaatgaactaagagcctattagactgtttaaaaattgctatattatactgatcttaaaagacaaaa
attgtttgatgtatttcataaaacacctagcacaatctttgggattttttaagtgttaaatttgattagg
tttattttatttgtcagaaattctatgtatttatgatgtacaacatgatgttttgaagtatatatacatt
gcagaatggttaaatcccgctgatttaacatatgcattacctcaaaaagttagcattttgggagaaaaca
acacttaaaatttactcttagcattttacaaagatacaatatagtgttaactgtagtcactgttatacaa
tcgatcttctgtatttattcctactacccaactgatactttgtattctttgaccaacatcttcccatccc
acccctacccccagcacaatctttgaagtcttggtaactttactgaaggtattgaatgtttattgtagtt
gttgcctttttgaaagtacgtaaataacatctttgcattgtagaaaaagttcttctttatagataaacat
gaagaaatgaagaaaattgaaatcattcataatcctgcggctttgagataactgatgagaacagttttag
attttttaatttattctatctgttctttttacaggcaaacgaagatttaatattaaattgtggaagacct
tcactgattgttttaactgtctgcctatagcagccattgtggatgagaagatcttctgttgtcatggagg
tagactagtaaatttgccttacagattttttttttcttctattattcggaaaatacctataataatcaag
aggctgtgggaaaacaaagcagtgcttgtatgaaaactcaagtgattaaaactgttttaaacatggctaa
acctcttttaaaaattagaaatttatttataaatgaaaacgtaatgcttctttaaatacagtttcacata
taaggagtgtaactagagttgtttgagaaagtctcctttgcctcaagttttaacagaaagacacccctgc
tatttattagtaaaaatgagaggatatcttcctgcctatattgaaatctttcatcagaatccaaataaaa
ttgagtttcatattcacatatatcaagaattctaggcttcatataatatatttgctttagtattaacaat
aaaataataaaataatctttggcagattggtaggttactctgtatggcttatgaaataagcttgaagttt
tttgaaagtttatttgaaaaattaatgtatatgtctaacacctagctagtagatagtgtatgatctgacc
atcttgaagagttaaaaacctgttgctaagatacatttatgaaacagataattgagtaataaaaggtatt
taatatgtcagaacttattaagacactatgagaagtcaggaggaggaaagactgggtacatgtgtgtatg
gaggcatgggttttagaggataaataggctttggagaagttaaaaggacttgagacactctttgttgtct
agtttagggaatgtgtaagaactggcctgactagaaggtcaagtacatgctgggaaatgatggaatcaga
ttatggagatttgactattgagcaacggagtttgttgagtgatttaataaccgtattgggtttttgtaca
gggaagtaatttaatggaaatagtgtaagaaaattagtttttcatcaatatacgggaagaattaaagtgg
aaaaacgctgaaaagatagatgcacttgcctaggcccgaggtaataaatggtttattctctggcagaaca
taattcactatattaattgaaatatgttttccagtagacattaaacacgtaatgaattgtagttcttgta
cattagtgtagctaaggatagaagtgtagcagtatggatacgaatgtttagcttcattccttgtgatgat
acttggattgcctatcagagagaaataatagcttatataccacttcaaagattgcagagtatttttatga
gcttttcctgttaatccttctgatcaatagtttatgacttcatgaagataatgaacatgttggagtggga
tgtgaaaagaaggtcttagggaaccaagtgaggcactttcacttagaagttaaaatgtttaagattagtg
gtaagaactggcaaaagtggtaggaagggttgcagttgattatgtttctatgaaggccagggaatgattg
gaaaaaagaatggtagttttttttttccttgggaagtcttttgaaatataaaggaatttcatttccctgg
tagggaaaaatggaaaaaaacaattataatgctgtattatatcaaagtggaattcctaataactctgtgt
tgccctttaagacattaaaaggggcagggcgcggtggcccacgcctgtaatctcagcactttgggaggcc
aaggtggttggatcatgaggtcaagagatcgagaccagcctggccaacatggtgaaaccccgtctctact
aaaaatacaaaaaattagctggatgtggtggcgcgcgtctgtagtcccagctactcgggagactgaggca
ggagaatcgctcgaacccgggaggtggaggttgcagtgagctgagatcacgctactgcactgcagcctgg
gtgacagagtaagattctgtctcgaaaaaaaaaaaagacgttaaaaggaacacttttcagtatctttaat
ttcacatagtgaatctattttgattaaatgctttttattcggtgactgttaaaatattaaaatttttagc
tgttagtactatgtctcatctttttatttataggattgtcaccagacctgcaatctatggagcagattcg
gagaattatgagacctactgatgtccctgatacaggtaagtgtagagagaagtttaattgttttgtgtaa
agtgttttcatatttgaacttgattacatttagtggaagtaggattggcttatgtaataaataaaagagc
ttttttcatagtcaataagccagctacagattactttatttgcagtaatacacaatttttcactttgcac
tgatcctcaaaatgcggaacctttttatttatagtcataatgtagttatttgatcacaaaaaagattttg
ctgttgagtccttatacctatttttctaaattccctcattcatactagcattggttatgttcttcaaaaa
tttgttaaattagacaaaagttttttaattaaaataatctattctttgcttcttaggcaaggatacaact
accttcttttcaaatgtctagtagttttttgttattttctatgtcctctatttgaaatttttagagcttt
ttttcaagggggtgtgggagggatttcaatctgttgcccaggctggccttgaattcctaggctcaagcaa
tcctcctgcctcttctgagtagctaggggactccaggcatgtgccatacctggcttaaatttttagagct
cttaaggctctagagacctgtggtcagttttgtgaaaattacattggaaatctctgggaaggctgggagc
agtggcactcacgcctgtagtcccagcgctttgggaggctgaggtgggcaggatcacttgaggtcgggag
ttccagaccagcctggccaacgtgatgaaaccccatctctactaaaaataacaaaaattagctgggcatg
gtggtgcgttcctgtaatcctagctactcaagaggctgaggcacgagaattgcttgaacctgggagacag
aggttgcagtgagccgagatcacaccactgcactccagcctgggtgacagagtgaaactgtctcaaaaaa
aaaaaaaaaaagaaaaaagaaacaacaatcaccacaaaaaatgctgctcagaactacacatcaaccatta
taaatggtatagatcctctgatgtcagtagccatgtaatacactgtatgttgtaataaattgtgttagga
gcttttttttttttttttttttttttttttgagacagagtcttgccctgtcaccaggctggggggcagtg
gtgcaacctcggctcactgcaagctctgcatcccgggttcaagtgattcttctgcctcagcctcccgagt
agctgggactacaggcgcccgccaccacacccggccaattttttgtatttttagtagagatggggtttca
ccatgttggccaggatggtctcgatctcttgatcttgtgatccacccaccttggcctcccaaagtgctgg
gattacaggcgtgagccaccacacctggcctgtgttatgagcttttttacataacacgacttgttgtgtt
gattattttttaaagtatgcattttatcacagtttttgattgtaattatagaatacatttgactaaacct
aacaaactggtatttaaatagtattttatttaaaatgctgtttttaggcctggtgcactagttcatgctt
gtaattccaacagtttgggaggccaaggcaggaagatcacttgaggtgaggagtcgagaccagcctgggc
aacataacacccatctctacagagaaaaaaaaaatttttttttttaattagccacatgtcgcagcaccgc
ggctactcaggaggctgaggcaggaggatcacttcagcctgggaagttgaggctgcggtgacctgtgatt
gtgctcctgcattctagcctgggtgacacagtgagaccctctctttcaaaaaacacccaaagtgctattt
ttaaaataaggtataatataaatatgtattactggtcattcttcagaatatctgtttatagttcagatgt
aaactgatctagtgtgatatgctaggcttctgtttttagcatgttttatttattttagcgtattttaatg
agaagttttcaagatgctgaaaacagttattttgatctttgatagtgtctgcaacattcagaaatttaat
ttataagttaatacattatttgaagcagcctagttgattgtgatgctagctcattaaacaataaatcttg
actctgaaattctttttgagacagaatttcactcttgtcgcccaggctggagtgcaatggcctgatctcg
gctcactgcaacctccacctcccaggttcaagcagttctcttgcttcagcctcccaagtagctagaataa
caggttcctgccactgtgcccagctaatttttgtatgtttaatagagacagggtttcaccatgttggcca
ggctggtcttgaactcctgacctctggtgatccgcccacctcggcctcccaaagtgctgggattacaggc
gtgagccaccatacctggcctgaaattcttatctagaccctgttaaggttaaagaattccagattgttgt
tgctaccattatttgatttttttaatgcatccagagtgtcatgtttcctaaacctcaaactctattctaa
ttttatacagttttattgtctcactgaaattatagattcttgagatcgagaccatcctggctaacacggt
gaaaccccatctctactaaaaattcgctgggtgtggcagtgggcgcctgtagtcccagctactcgggagg
ctgaggcaggagaatggcatgaacccgggaggtggagcttgcagtgagccgagatcgtgccactgcactc
cagcctgggtaacagagcgagactccgtctcaaaaaaaaaaaaaaaaaaaaagaaattatagattcttaa
taccttagctttctactggaaagaggctggaaagtatgaaagcattattaagcactgggtgggtgtatat
tttcgaagagtcagcttcaaccagaaagggcttaagaattgagactgactattccagtggattttgtgtg
ctagtgaatttcttaaaacaacatacagggagcttgggatatatcaatgatagagattgaatacattgcc
actaattctacaaatggttttataatgcctctatcaagatatgatgtactgtaactgaaaatgaagatgc
cctttcctcctaaaccatactatgatgtttatggattatttaaaattcttaaaactgacttaactcttta
aaaattatcttcttggctgggcgcggtggctcacacttgtaatcccagcactctgggaggccgaagcggg
cggatcacgaggttaggagatcgagaccatcctggctaacacagtgaaaccccatctctactaaaaatac
aaaaaattagccgggcgtggtagcgggcacctgtagtcccagctactcgggaggctgaggcaggagaatg
gcgtgaacccgggaggcagagtttgcagtgagcagagattgcaccactgcatcccagcctgggcaacaga
gcgagactccgtctcaaaaaaatatatatgtatcttctcttagccactgcaattcttagaaaatcaccat
caccatctcaacatccacccttcttttaatatcactacagttggacttttacccactccgttaccctcaa
tcaccaagaaacttgcattgaggtctgatgatttaattgtaagtggcacatttttggtcttcgtcctact
gcccttgttcatggcacatttcctactttaggacgattgtttcccctttcgggatacgtttactatacta
agccctttgtcttgatcattccttctcaatacctaatgttcattctccctcatctacctgcctattaaat
gtttgaattcctcagaattcatttgctggtcttctcactttgtattcctaaggtggtctgagtccctctt
aagatttccactaccattaatatgatgacaatactcaagtaaatatttttaaccctgacctcttttccag
ggctccaaacattaccaaagtcaaggatctgatctgattttgttcttccacatttaaaaaaaaatcctca
gggtctcagtagaaaatgtttttttttcgctcaagtgatctgtagatcaaatacaattccagtgaaatca
caattgagttttttatggaattaggcaattgtttttaatgtttatgtggaagaacaaaagatcaaaaagt
aaccaagacaatcctgaaaaaaaaatggaggatactagccctaccaaatataaagacttattaaaaagct
ctagtctgttattggctcagggataagtaaaatagactaacagaatagactgtggtgctcagaaacagtg
gaaccgtaaggaaaggatgaactgttgaggaaataccactgggttgtttggttattcatatggagaaact
acccaaaagatgaacagccttttccatgtattgttgtgctgtaagtgccatgcacatatttcctatatgt
ggaaagggttggaaagtactaccttaagaggccatggaagattatatttgtatggtttgtatggttgctt
taacaaaagtataagttacataggctggaatacttaggcaagtgatggtcattgtttagtaaaaggtaag
agggcaagaaaggaactgaaatttgatgtagatgtgctatattctataaatctgtaaattttgttcttaa
ttgctgtatttctgtcctttaaaggtttgctctgtgatttgctatggtctgatccagataaggatgtgca
aggctggggagaaaatgatcgtggtgtttcctttacttttggagctgatgtagtcagtaaatttctgaat
cgtcatgatttagatttgatttgtcgagctcatcaggtatgaaatataaaactcttaagctttttctttt
ttctttcttttttttgggggcagacggaggggcagacagattcttgttctgtcacccaggctggagtgca
atggcgcaatctctgctcactgcaacctgtgcctcccgggttcaggcagttcttgtgcctcagcctccca
agtagctgggacgatagccgtgtgccaccacacctggactcttaaacatttattatttgttaacttcaca
taagccctctcattttgttttgaggaaacatatccaagaaagctaaatgagttgtacaaggaaagcagta
ggagaggtggcagagccagttcctggatctcacacttaacaatctagtgatttgttaaccttttatcacc
ctaaaagaggaaatgtttagaaatattttggggctgggcatggtggctcacatctataatcccagcactt
tggcaagctgagatgggtggattgcttaagtacaggagtttgagaccagcctgggcaacaaaatgagatt
ccatctctacaaaaaaaatcaaaaatttagctgggtgtggtggtgcgtgcctgtggtcccagctacgtgg
gaggctgaggtgggaggatcgcttgagcctaggagatcaaggctgcagtgagccatgtttatgtcactgc
tctccagcccttgtgaaagagctagacccagtctcaaaaaaaagaatgtatttttgtagtatacagcaaa
cctaacaaaattttcagtgcttctttgaagctttgtagatatgatttagatttttttttttttttttttt
tttgagatggagtctcactctgtcacccaggctggagtgcagtggcacaatctctgctcactgtaacctc
cgcctcccaggctcaagcgattcttctgcctcagcctccggagcagctgggattacaggcacgcaccacc
acgcctggctcatttttgtatttttagtagagacagggtttcaccatgttggccaggctgcccttgaact
cctgagctcaagtgatctgcgcctcagcctcccaaagtgctgagatgacaggcgtgagcgtgaaccactg
tgcccagcctcatttttatataatctttgtgcataatggcgattttgataagagcaaaccacactttaaa
aaagtatttttgagaccattgaagaatttgattgtggctcagtgcagtggctcatgtctgtaattccagc
actttgggaggctgaggcaggcagatcctttgagccccaggagctggagaccaggctgggcaacatgaca
aaactctgtctccacagaagatacaaaacagccagatgtggtggtgtgcacctgtagtcccagcttgctt
gggaggctgtggcaggaaagtcgcttgagctcaggaggcagaggttgcagtgagtgtagatcgtaccatt
tcactcctgcctgtgcaatgggtgtgaaactcagtctcaaaaaaagagaacatttgattgtagacttggt
aataggtatgaattatttaatcttttaattttttgtttttgtttgaggccgagtctcactctgtcaccta
ggctggagtgcagtggcatgatctcggctcactgcaacttccgcctcccaggttcaagtgattctcctgc
ctcagcctcccgagtagctgggactacaggcgcctgccaccacgcctggctaatttttgtatttttagta
gagacggggtttcactatactggccaggctgatctcaaactcctgaccccaggtgatctgcccacctcag
cctcccaaagtgctgggattacaggtgtgagccactgctcctggccggtatcaattattttagctgtttc
ataatcattaattgttatgtaagaagaatgtctgtgtttttccaggaatgcatacttgagtgtgtagagg
taaaatgacatgcctgggatttgctttaaaataatatcagcaaagagaaaaggagagggagaagataaac
aaatgtagcaaaatcatgaaaagtattgaattaggctgatgagttaatgggagttaattcaagtcgtctt
tatctacttttgtgtatatttgaaatttttcattataaaaacatttatacagttcagggtttttggttgt
cttttatgtccgccaccactaactagattgtgtactctaaggaagggattacaacttgtttttgaatctc
tgttgctttatatagttgctgacattgtagcatagtgtatcatgcacatacagtgttgaataaattaata
aaagcgtatttaattaagtatatgctgtcccacagtacttccctttctttctggagctttactccccatg
acacttactactgctgcctctttctttgatcctaagtgataaaaatgactggctttttattctttgctta
ttgtatattacatgccacttttatttagcaggatttcttggtggttccattgaaatgaggtcatgttccc
acatcctttgattttggcaaagtagttttttgttgttgttgtttgttttttttgtgagacagagtctcac
actgtttcccgggctggtgtgcagtggcaccatctcggctcgctgcaacctccacctcccgggttcaagt
gattctcctgccttagcctcccgagtagctgggactacaggtgtatgccactagcctggctagtttttgt
atttttagtagagacggggtttcactatgttggcctggctggtctcgaactcctgacctcgtgatctgcc
cgcctcggcctcccaaagtgctgggattataggcacgagccacctcgcccggcctgacaaatagtttttt
aaatcagtaaaactgtgtttatgattttgataaatatgccttctattaaataacaaggcagaattgctaa
gtttcctaactaaaatgacccctgataatcagtttttttcagccatagatcttcattcactggtcttttc
aaaaataaatgatgatttttaaaaatcaggtttctgaactacgctagtttgcatggtcttttgttttcca
gaaactgccaaaatccttctattgtgcttattttcaaataattgtctctaaaattctagaaatattccct
ttaaaaaagaaaatttgaaagaatgaacaagtaagcaaactaaaaaaccaagaactaaaaacatggagtc
catgaccagcctgggcaacacagtgaaacacgaaaccctgtctctacaaaaaaatacaaaaaactggctg
ggcaccgtggctcacgcctgaagtcccagcattttgggaggctgaggcgagtggatcacctgaggtcagg
agttcgaggccagcctgaaaaacatggagaaaccccatctctactaaaaatataaaattagctgggcacg
gtggcacatgcctgtaatcccagctacatgggaggctgaggcaggagaatcgcttgaacctgggaggcag
aggttgccgtgagccgagatcgcgccgttgcactccagcctgggcaacaagagcaaaactccatctcgaa
aaaaaaaaagaaaagaaatacaaaaaaattagccaggcatggtggcacacacctgtggtctcagctactc
aggaggctgaggtgggaagatctcttgagccccaggaagttgtgactgcagtaagccaagattgcaccac
tacactccagcctaggcagcagagcgagaccctgtctccacacaagcacatacacagagaaacaactaaa
aacatgagtgccatttgctatgtggagcaggggtcacagggattagataaggtggctatggtcttaggag
aaaaggttcatctaaattgtaccataaggtaatttcagcagaagcatgttcctagtactgaggtaggagt
tattttaataagtaaatgccttcgtaattgcttttttatattagcaagtggtgtcggataattttgctaa
ttaaaagatacctaaaataaatttaaaagtaatcttgcccaccgtaaataaatataaaatatttacatat
tattgtgttcctcaaatttgtagactgagagaaaaattgtttaaattactcagaaattgacaactggttc
tagaaatgttaactcatcaaggccaggcacggtggctcacgcctgtaatcccagcactttgggaggccga
ggcgggcagatgacctgaggtcaggagtttgagatcagcctggccaacatgacaaaacctcatctctact
aaaaatacaaaaattcatcgggcttgctggtgggtgcgtgtagtcccagctatttgggaggctgaggcag
gagaatggcttgatctgggaggcagaggttgtagtgagccgagatcatgccactgcactccagcctgggt
tgacagagcaatattccatctcaaaaaataaaaaataaaaataaaaatacagtctgttggcagttaattt
aaggagcatgatccatgtaaattaaactcaacttagtaatgttttcagtgtgtagccagtagtgttttca
gtgtgtagccatgtaataataatacatattttaaaatcaaagcaagaagtaagctcttcgcaaagtcgtt
cattgtctggttgttgctcatttaattaaggattctgtagttggattataatgccctcatcaaagtaata
attgatgtccctctgaaaaacacgtagattaattaaaataaaattaaatttaaaacttaaaaacaaaaaa
agtacttagtgtcccagctccctgtagtcatgatgcctattagtatgttttgtttcatggggagccttgc
aaatggcatgatttttaagctttcaggagtggtgaatttattattccaattaagctaaattgtttccatt
atagaaaaggcacaccagttttaatgttctaagcaaagtttaagaataaaaatcacaacttccaaaacaa
aacagggtggtttgggggtgaggtggggcacagtctttgccaccttaaccttaattagtatagatggttc
agaattacccaccaataaatgttttttcttctgacatttcctttgacaggtggtggaagatggatatgaa
ttttttgctaaacgacagttggtaaccttattttcagccccaaattactgtggcgagtttgataatgctg
gtggaatgatgagtgtggatgaaactttgatgtgttcatttcaggtatgatgtaaacataaatatataag
aactagaaatctaataaaaactattatcagaattcgttttagatttgtgaagtttccttgagcaagtgtt
gaaagtctgtttaattcagaagtgattaccactcaaactcattagatatttctattttcagtcacaaagt
ttccagcatttgtggtatatatcaaatatgaccgttttagggaacctaagttacgtcagtcattcttgat
tcagatgttttaattgttaaaattcctctatgaccttttgaatagtggcttactaaaagaatgtgaatgt
aatgaatagtttaggcttttgttttcttttgcaaagtaaatgggtggaacagacactgggaaaagaatta
caaactcagatgaaaataaagtaaaatagttccctggctgggcacggtggctcactcctgtaatcccagc
acttcgggaggctgaggtgggcggatcatgaggtcaggtgatcaagaccgtcctggctaacactgtgaaa
ccccatctctactaaaaatacaaaaaaattagccgggcgtggtagcatgcacctatagtcccagctactt
gggaggctgaggcaggagaatgtcgtgaacccgggaggcagaggttgcagtgagccgagattgcaccact
gcacgctagcctgggcgacagagcgagacttggcccccaaaaaaaaatagtttcctgaggaaaaaaaact
ttcaaaataatactaaactacccgtttatgtcacaggaacatttatttatttatttttttcttttgagga
agcacattgtataccatgtacggggggttgtttgctcacttttattataatatttttattaatttatgta
cgtactaggttcaggggttacatgtgcaggtttgttccatgtgtaaattacatattgctgaggtttggtg
tatgaatgatcttatctcccaggtagtgagcatagagtttttcatttcacattccccctcaccctttcca
ctctagtagtccccagtgtctattgttgccatctttatgtccatgtttacccagtgtttagctcctgctt
atgagtgagaatatgtggtatttgattttctcttcctgcattaattggcttaggataaaggcctccagct
gcatccatgttgctgcaaaggacctgatttccttccttaattttatggtcgcatagtattccatggtgta
tatgtaccacattttctttatccagttcactgttgatggacatctaggttgattacatgtctttgctatt
gtgaatagtgctgtgattaacatacaagtgcttgtctttttttgtacatacccaaagtagtgggattgct
gggtcgaatggtggttgttttaagttctttgagaaatctacacactgctttccacggtggctgaactaat
ttacactcccaccagcaatgtttgaacattcctttttctccacaaccttgctgacatctattatctttta
actttttattaatggccattctgactggtgtgagacagtatcttgtggtttgatttacattaatagtgat
gatgagcattttttcatatatttgttggctgcacgtttgttttttgagaagtgtctgttcatgtcctttg
cccatttttaatggggttgttttttgattgttgaattaaatgccttatagagtctggatattagaccttt
gttggatgcatagtttgtattttctctcattctataggttgtctgtttattctgttgatagtttattttg
ctgtgcagaagctctttagtttattttggtcccacttgtcaatcattgttttggttgcagttgcttttgg
ggacttggtcataaattatttcccaaggacagtgtccagaatggtatttcctaggttttcttctagggtt
tttatagtgtgaggtcttatatttaaatctttaatccatcttgagttaatttttgtgtatggtgaaaggg
gtccagtttcagtcttctgcatatggctagccagttatcttagcaccatttatcaaataggcagtctctt
ctccattgcttgttattgttgactttgttcaagatcagatggttgcaggagtacagctttatttctggct
cctccatcctgttccattggtttatatgtctgttttggtaccagtaccatgctgttttggttactgtaag
ccttataatgtaccttgaagtttggtagtgtgacgcttctggctttgttctttttgcttaggatgctttg
gctattcgggctctttttcaatttcatatgaattttagaaaagttcttttctaatgctgtgaaaaatgac
attggtagtttgataagaagagcattgaatctgtaaactactttgggcagtatggagattttaacaatat
tgattcttctatccatgagcatggaatgtttttccatttgtttgcatcacctctgatttctttcagcagt
gctttgtagttgtagagatctttcacctccctggttagctgtattccttgatattctttttgtggctatt
gtaaatgcagttcttgatttggctctcagcttggatgtttttgatgtatagaaatgctactggtttttat
acattgattttgtatcctgaaactgattaacaaagttgttcatcagttccaggcacgttttggcggagtc
tatggggttttctgtgtatagaatcattatcgtcagtaaagattgtttatcttcctctcttcttatttgg
atgccttttatttctttatcttgcctgattactctggcagggactttcagtactctgttgaataggggtg
gtgagaatggtcatcctagtctcttgttccagttctcaaggggaatgcttccagcatttcccttttcagt
acgatgttggctgtgggtttgtcagatggctcttactattttgaggtatgttcctaatttttcagtgcct
aatttgttgagggtttttaacatgaaagggtgttgaattgtattgaaagcctttctgcatctattgagat
gatcatatggtgtttgtttttaattttatgaggtgaatcacatttattgatttgcatatgttgaaacctt
gcaccccaggaataaacccttcttgattgtggtgagttaactttttgatgtgttgctggattcggtttta
gtattttgttgacgattttgcatctgtgttcatcagggatattggcctgaggttttcttttttccttgtg
cctctgctaggttttggtattacaatgatgctcacttcgtagactgagttaggggggaggtcctcttcct
ggattgtttggaatagtttcagtaggattggtactagctctttgtacatctgatataattcagctgtgaa
tctttctgggccagggctttttttggtttataggtttttttattactgattcagttttggaacttgttat
tgttctgttcaggattttaatttcctcctggttcagtcttcggagtttgtgtgtgtttctatgaatttat
tcatttcttaggaggtcatgtgtgtttctatgaattcatttcttgggagcgcatgtgtgtttttgtgaat
gtattcatttattctaggtttcctagtttgtgtgcacaggtgtttataatctctaggtattttgtatgtc
tgtggggttggttgtaatgtcacctttctcatttcagattgtgcttatttggatcttttcttaatctagc
tagcagtctatcttgtttattcttttgaagaaccaacttttggtttcaacatttacatttttaaaagagg
ccagttttagctttagtgtgctcctacaaatcattcttgttcactgagtaaaacagcttcagtactttgc
ttttgcaaaggctattctatgtctttgatgtacacacactgtatttctcttaaaatgcattggggctttc
ccattcctctttctctttatcataatatactgtttccatcaattagtacaattgaatggtaaatggttga
ttgctcatttacaaattaaatatagtaaccataccttatccttccagcagttgtcatagtggtgttggat
taatatattctcttcactaaggcactattatgttctaaatagtttttaaaaaatactttgtacatatttc
tatactgttttagttttggaaaatcatgacttttttatgtataatgtaggttaaagctctatttacatcc
accagaatgagtgtagtaaaaaagccagactgtatcaagttttggcaaggatttggaggcattagaatct
tcatacattaatggtgggaatagagttctcatatgatgcagcagttttacctctaggtatatacccaaga
tatatgaaaacatgttcacacaaaaactgttacataaatgtgcattattccttatagccgaaaaagtcac
acaattcagatgtccatcagctactgaatagataaactgtggtacatccatacaatggactgtacaatgt
acaactcagcaataaaaaggaatgaaggactgatacttcaaaactggttttgaaaacctgagtgaaagaa
gccagtcacaaaagaccacatattatatgatgccagttatatgaaatgttcagaataggcaattctatag
agacacatagtagattagtagttggttagagttagaggatgtggagagcaggaatggggagtgactgctg
atgggaacaagctttctttttggggtgacaaatgttctaaaattagatttatatcaataaagctgttttt
taagtaaaaagatctatttatacataaataaataaggtctgaagggtttgtgacttgtaggttagttacc
ctcctaagttactgtatcagaattatgatgacaacatagatcttctgactcttaaaccagtgttcttttc
ctcacaacagagatacactaagttcaaaaagttttttacttaaatctaaggattggatagttgatagagt
tagtgtacttttgttattaaaatatgagtggaatgacaaagttcagacatttgcacactttaagaaatgt
ttatggaactttgctattctacatttttattgcatttttgcagtttatgccatttattgaaaatcacaat
tgtaacaattttcttaacttctgtacatgaaaaaataacattatttgttaataatttactttaagatatt
gaaaccatctgaaaagaaagctaaataccagtatggtggactgaattctggacgtcctgtcactccacct
cgaacagctaatccgccgaagaaaaggtgaagaaaggaattctgtaaagaaaccatcagatttgttaagg
acatacttcataatatataagtgtgcactgtaaaaccatccagccatttgacaccctttatgatgtcaca
cctttaacttaaggagacgggtaaaggatcttaaatttttttctaatagaaagatgtgctacactgtatt
gtaataagtatactctgttatagtcaacaaagttaaatccaaattcaaaattatccattaaagttacatc
ttcatgtatcacaatttttaaagttgaaaagcatcccagttaaactagatgtgatagttaaaccagatga
aagcatgatgatccatctgtgtaatgtggttttagtgttgcttggttgtttaattattttgagcttgttt
tgtttttgtttgttttcactagaataatggcaaatacttctaatttttttccctaaacatttttaaaagt
gaaatatgggaagagctttacagacattcaccaactattattttcccttgtttatctacttagatatctg
tttaatcttactaagaaaactttcgcctcattacattaaaaaggaattttagagattgattgttttaaaa
aaaaatacgcacattgtccaatccagtgattttaatcatacagtttgactgggcaaactttacagctgat
agtgaatattttgctttatacaggaattgacactgatttggatttgtgcactctaatttttaacttattg
atgctctattgtgcagtagcatttcatttaagataaggctcatatagtattacccaactagttggtaatg
tgattatgtggtaccttggctttaggttttcattcgcacggaacaccttttggcatgcttaacttcctgg
taacaccttcacctgcattggttttctttttcttttttctttctttttttttttttttttttttttgagt
tgttgtttgtttttagatccacagtacatgagaatccttttttgacaagccttggaaagctgacactgtc
tctttttcctccctctatacgaaggatgtatttaaatgaatgctggtcagtgggacattttgtcaactat
gggtattgggtgcttaactgtctaatattgccatgtgaatgttgtatacgattgtaaggcttatgtcact
aaagatttttattctgattttttcataatcaaaggtcatatgatactgtatagacaagctttgtagtgaa
gtatagtagcaataatttctgtacctgatcaagtttattgcagcctttcttttcctatttctttttttta
agggttagtattaacaaatggcaatgagtagaaaagttaacatgaagattttagaaggagagaacttaca
ggacacagatttgtgattctttgactgtgacactattggatgtgattctaaaagcttttattgagcattg
tcaaatttgtaagcttcatagggatggacatcatatctataatgcccttctatatgtgctaccatagatg
tgacatttttgaccttaatatcgtctttgaaaatgttaaattgagaaacctgttaacttacattttatga
attggcacattgtattacttactgcaagagatatttcattttcagcacagtgcaaaagttctttaaaatg
catatgtctttttttctaattccgttttgttttaaagcacattttaaatgtagttttctcatttagtaaa
agttgtctaattgatatgaagcctgactgattttttttttccttacagtgagacatttaagcacacattt
tattcacatagatactatgtccttgacatattgaaatgattcttttctgaaagtattcatgatctgcata
tgatgtattaggttaggtcacaaaggttttatctgaggtgatttaaataacttcctgattggagtgtgta
agctgagcgatttctaataaaattttagttgtacacttttagtagtcatagtgaagcaggtctagaaaat
aagcctttggcagggaaaaagggcaatgttgattaatctcagtattaaaccacattaatctgtatcccat
tgtctggcttttgtaaattcatccaggtcaagactaagtatgttggttaataggaatccttttttttttt
tttaaagactaaatgtgaaaaaataatcactacttaagctaattaatattggtcattaaatttaaaggat
ggaaatttatcatgtttaaaaattattcaagcactcttaaaaccacttaaacagcctccagtcataaaaa
tgtgttctttacaaatatttgcttggcaacacgacttgaaataaataaaactttgtttcttaggagaaaa
tgattctgtaattccagtgtcactaatttatattgttctttcctctgatttttttcaggttagtgatttt
tttgtatacaatttaatccaaatgttatgacattcagaaatcatgaaacacagtagatatctgttataat
gtggtgtatcacatggattataaagcaaagttatggtcgatttctattcttgaaagaatcaactacagtg
aatcctttgcatttgaagccttaacatgcattgctttaattttgcccagggacaaattttaataatcagc
aagactggtttgtgcaaagcgttgagtcatcaggtatttagagcctagccagctacccagtatccatgct
gccatatcccttcattgtaaaaagtacctaaacattcgtgaaatgattttttttagctgaaaaatgctgg
caagaagaattttaaagcttaaaataggtggtaaatttgaagtatgagtgtgttcacgagaaacataggc
ttttcaaaaaaatttttattcaaggcaaagcaaggaacatcttgagatatgtctcaagaatataaagatg
tattattttaagccaaggagctgaaatatatctcagtttataaattcaggtatattctttttgtctccat
ggcaaccataacttttgaaccaaaaaaaattgtttttacatctttatgctgaaaatgtgtttagattagg
aatatggtcgggctgaatttgctgttgctccctaaccaaatccacctcttgttttccttgtgagtccatg
gctaaatcaaagctgcccctgagaagagacttaatccaagcctgattgtactagtggcatcacttagaag
taggctttccctcttcctagtagatctcaatgttttataattccttaaaacagctgaaaattgggacaac
atactttacgcaatgaacagtagttaaataggaaataaactagttccatataagtatacacctagagttt
taattacctttataatgtttcttaaaagtgaaacttagatacaattgtgattggatacttagatactaag
tgaaacttagtgtaacaattttgatctgttaaattggattttacatgtacatttgaatgccagaatttct
aaataaatcccctggttaggaaattttaaaagtcaaagcttgttttcttcaaccactaccttctacattg
gttgacttagaccgtaagctttttaagtttctcattgtaatttaccttctcatgcagattgctgatgttt
tattaaaccttatttttacaaaaatga
Human PP1 subunit C (SEQ ID NO: 9):
aaggagtcggcggccattttgttcttctcgtggttccagtggggagagaaggaggaagtagggagcgggg
tggcaggggggggacccgccgcggctgctgccaccgccgccaccaccgcctctgctcgtggcgtgggaaa
ggaggtgtgagtcccgggcgcgagccggcggcggcgccgctgcgggagggtcggcggtgggaaggcgatg
gcggatttagataaactcaacatcgacagcattatccaacggctgctggaaggtaaggggggcggcgggc
ggctgaagggggaggcgggcctgagggcggcaggcggccccgggggctcgagggaggggagttgggaggc
cggttgggcttgcagcaccgtggggagtgagctcctcgagttggcccacgggccggggactgcagccgga
gggaggccagcccgggattcccaacttgtcgccagctgcggaccctccaggctccgctcattcattgcac
gaggatgcaggcttcctcccggccgtggaatttgaaaactccgctccccacccccacgtttcccgcccct
acccgtttgcccccgcgcactggacgggtctttggagcgatggttggggatcccagtccagcctcctgga
cgacgtgtgccgtgctgtgcgagatggacttttcggtgctcgtcctctcggaaggcagtttgatgagggc
atgagttccgccgggtttgggggctcttttcctgcttgcttttatttttatttttcgtggttctggggtc
ttggggggcccatttcttgttacttggtaacaagcgttcatgtctcaagaggtgtgagaacctgtcttct
cctgccctccaaatccttttaagttgttctaatcttggagaggtctccgcgctcttttcctgagcgtgca
tctgaagaggaagccaacgaggctgacgctatcattatctttccaatgtgccaggctctgtcttaagcac
tttgcgtccatgaattattactgtaatcataactgacgcttctaaagcgcttactgtgtgtcaggcattc
tttgagcacttaagacagatttcatttaattctcccacccattgcaagatatttgcaattttttttttt
ttgttattttacagaagaaactgagatgaagtcacttaaccaaggtcacacagttagtgacagttggatt
tcaaatccacatagccgggagctagaaccctctacagacaacactgtcaccttatgtaggaaggaaggtg
gccagtggttaagtaaaggttaatagaaagctttaaatttcggctcatctaagacttgtgaggtgcctgg
caattgattcatgttgatgtgggagaggggcgaggttcagcttttaaagacggaagctgaaatgagctgc
tttcagattcaagggcatgtcaacggatgctaacatcttatccaagactttgtaagaattcagtcatatc
ccctaacttggacacttcataatgggaacttggcagtttcaactgctttttgcctttatacagtcctaaa
atattgttggactataaatgctgtgagtggtggagtcgagggcatgaacagttggacagctctggtgtta
gaatttacagatcttttaaagtaagttattaaaatgaagctcctgaaaatatctcgtccttttcttaatc
tgtgcattctttccttttatgctgtctttaaaaaaaaaacaacaaaaaaaacactaaatgtcattaagga
cccctgaaatgctcttgaaatgttattgcaggtgaaaaccttgtattgtaggtgaaaaccttgtcattct
tttaatgccaggtatttatataagaaccatactttttcatactagttaatatagttctttaatttattcg
aggcagcagttggtcagaaactgattaaaatagtttgctgtaagaaaggaactttttgttaaatattttc
tttctaaattttaaaattctgcttatctctgcagggatatgtgtggctttatttctctgaggaaacagga
aaagttggaatttgcatggattagaaacattagaattttaagatcatctaaggcttgctaatttaagttc
tggtttgcccataactgcctctattcataaacagcttttgttaattttgatctttaaaattgtaataagt
actttgcctggtaagtaatgatacatctaaaatgagatcattgtttcttaatagttcctcttgttatgtg
gcacttgaaaaccaggttttcttatatttcattttttgctgaaattaataaagcagtgcattttaatgcc
ttgggaataggctaactgcaaagtatacttaaatttctttttttctcgtgggtgttaaattggcagagaa
acagttgaagatgatccattgaggatatgtagatatctaagttaaatgatgttggtaataaattctgttt
tacaattttttatttaaaaaaattaaatttaataatttaaaatagaaatgagagtctcactgtgttgccc
aggctgttctcaaactcctggtttcaagcagtcctcgcacctcgtcctccccaggtgctggggttatagt
catgagccaccgtgcccggcctaaattttattcttttcataatgcccccaaaaccttttttttccctaaa
taagatgtaagtatatctgaaagtagagggaatatgacttgtgactttggccagatttttttacgtgttt
agaattgatcttttaagcttgcgagcagtataacctagttgagaagtagctgagtaatagtactaaaaag
ctaactgaaaagtgaagtcttaagaaaaacagcatgttggtttagatcttagtcactaacctttgcagtt
cttttcttctctgaactctgagtatttctgtaagcgaggcatcacctctgcaacccaccatggatgcttt
tatccaagctctgccttgctgatgataaaacttaaggttgttaactccttagtataactgatgttagtta
tttaaataactacacagtaaaacggcattttgagagaaaataagccagctgttgatttggtctgatgtat
tgaagacaagtgagatttggggcagttttactttgtaaatgacctccttcaaggagccatggtggctagc
attttttttgcatagcaggtggttgtaacttacttcacatcttggtttgcaagaagcatctttgctttga
agaaggccattgtcaaagtgcaagaccatgtcaggctctgcagaattgcctgacttgttttagtgattta
aatatgatctctgccctggctcacagaaagtgcttacagatttaatcctctgatacataagggttgttct
gaaggtagggagtgttcagatattctgaaactctaagattgtttaaataacccatatgttcatctttttg
atgcagtataaaacgttcatattctggtgtttagccatccctggtcctaaagataataaaggacaaacat
aatggtttaaaaaggggaaggtttcttaatatgctaaccaaaaaaaaaaacaaaagtaatctttcctaat
gaacacctgtaattatttatttttttattttattttggagacagagtctcactctgtcacccaggctgga
gtgcagtggtgggatcctggctcactgcaacctctgcctcccgggttcaagcgattttggtgtctcagcc
ccctgagtagctgggattaacaggcatgcaccaccacgctcggccaagtttttgtatttttagtagagat
gaggccaggctggttttgaactcgtgacctcaagtggtctgcccgcctcagactcccaaagtgctgggat
tacaggcatgagccaccgtgcccagcctgtaattatttgtttacagagcaggtaatttttacactcacat
ccaagttgtcagtttctaattccctcattgataagaactctgatgtgagcccttgcccttcctttcccat
cagtatgttggtcatatgcccacttcctctcctcctaggcaaggtgggaaagctctgttggagacagatg
atctgcagcttgcaggagtccttttttaaaccacagcattgaaaaaggattactctccttgtgcccgcag
tgccctcttgcctgtcagtagcaggaggcagcagtgaaatctgacagaattgtgtgtgacgttgtgcttg
cgggggatgtgacacacaggagcgctctgcattctgagcctagggtgttcctcctcataactgggcaaat
ggacatgcagttgtagcccagcagtggtcagctgtagaaattagtgaggtgatttgatgtctttgaaagc
acagtcctattacaacctgtaacctgttgtgcagaatcacagtaccagtcagcccacttgactaggtgag
gattcctgatggtgaagcagggaaattattgatgttatgtggacttcaccatttaacttaatttacaatt
gaggccattagttgacctgagagattaaaatatgaaggagataatattctgggtgtcagtcgctttgtag
ataaagaactggaaagtcattcaagaccaagaaggaaggatttcatttccatggagcttatggaattcca
ggatagagtttgatattttaagatcatacctctgtatccaaagcatcaagaacagatgatgtgtttgtgg
atcttggtgatttgtctgaaatctccagcttccccacttcactggggtttactgtcatccctcttctaga
aggtagtggtcatcaccgacagtaaagcttacagcaaatgaaaactcaaaatgtagtctactcagtggta
tattaccgaaatgtgttcttagactgtgaaaattaaagtttttaatggattggctttactcgaggtgtag
aaaactcaagatgctttattgagaacagtagacagtgttttgcagccacatcctatctgccttgaaaaaa
atggtgtcacttttattttttttactagagactgggtctcactctgtccccaggctggagacctgaactc
ctgggttcaagcgattcttcctcctcatcctcttggctagccggatctataggcacacaccaccacacct
ggctaatttttttttctttttctttttttcttaaagacaggggtgttgcctaggctgttctttaactcct
ggccacaagcagtcctcccacctcagcctcccaaagtgctgggattacaagcgtgaaccacagctccctg
ccttggtgtcacttctcaatgcctgaagtgtaaccccatctgttaggaaaatcaggatgggagagggcag
ggtgtggaaacaattgtgataatcagaacatggaagtctttctgctggtgactttgcagagaaacttaaa
gggttcttttttcttttcttttcttttttttttttttttttttttttgagtctttctttgtcacccaggc
tggagtgcagtggcaggatctcggcccactgctgcctccaccacccaggttcaagcgatttctcctgcct
cagcctcagttgttgagtagctgggactacaggcatgtaccaccatgccctgctaatttttgtatttttg
gtagagatggggttttaccatgttggccaggctggtctcaaactcctgacctcagctgatccacccacct
tggcctcccaaagtgctgggattacaggcgttagacaccgcatccggcccaaaggattcttgataacatt
tttgtggtttatactttttgcagaaaagaactcagtcgtttatgggaatgcttaatgttggaagtattta
ctgttttttgttttggtttgagtcttattttcctaaatctgtcaaatatcatgaggctattgatttcact
gaaatgaatatgatgtggcatattgttaaaattatcacctactttttaacaggattattttaaaacagca
aaatcttcgttggcttatttttataccacttgtgcctttcttaggttctgaaacttcattagaattcagc
tagggcttttttagtatatgctatttctttaatatgtttttatttaaacaattaaggggcctttatgtgg
caaggaaaatacttttttattccttataaataatctcaaacttgtcttaaatttccttccatagtttcca
tctttgctttgctttttgccagcagctaactttcaaaagttagtattgcttatgagaaagagctggtgtt
gtaaataatcaccaatgctattttatttatttatattttttgagacaggatctcactctgtggcccaggc
tggagtgcagtggcatgatctcggctcaatgcagcatctgtctcccgggctcaagtgatcctcccatctt
agcctcccgagtagctgggactacaggcgcacgccaccatgtccagctatattttgtatttttggtagag
actcaatttcaccatgttgcccaggctagtctctaactcctgagctcaagtgatcctcccacctcggcct
cccaaagtgctgggattataggcccaagccaccacatctggctgctattcttttataattcaaattttta
ccagtttgttttcaataaaaatgtgtggaagtttttattattgctcgtgtcagtgatttgccagaaaaat
agatcagatacatttctacagagcaaccttcgcttttaacagtaataatacgttttcctgaatctccctt
tctagccagataaagaaatattaatacttctactgaaaagtcacatccttaggtctaagttggtgttgct
tttttgttgttgtttgtttgtttgttttgagacggagtttgtttgtttgtttgtttgttttgagacaaag
tcttattcttgttccccaggctggagtgtgatgatgcgatctgggctcactgcaacctgccccgtgggtt
ccagcgattctcctgcctcagcctcccgagtagctgggattacaggtgcctgccaccacgcctggctaat
ttttgtatttttagtagagacggggtttcaccatgttggccaggctggtctcgaactccttacctcaggt
gatccgcccgcttcggactcctaaagtgctgggattactggcatgagccaccgtgcctggccgagacgga
gtttctttcttgttgcccaggatggagtgcagtggcacaatctcggctcactgcaacttccacctcccag
gttcaagtgattctcgtgcctcagcctcctgtgtagctggggttacaggcgtgcaccaccatgcccagca
aatttttgtatttttagtagagatggggtttcaccatgttggccaggctggtctcgaactcctaacctca
ggtcatccacccatctcggcctcccaaagtgttgggattacaggcatgagccaccgtgcccaaccttctc
ctttggttttgaagttcaatgtagaacagattcttaattgggaattaagaatgcaccactagatgtagca
tcgcggcctcgtgtccttaagctctgtgaccttggactaagtcactgtaaaatgggagatgacagtgcct
gacttcctaggatcgttgacaggattgaatggcaatataaagcccttagtacagtgcctagtggataagt
gctcagtaaatgttaactacaacgagtaaataattctgtttgatggacattgaaaatagtgaagtgggcc
aggcacggtggcgcacgcctataatcccagctacttgggaggctgaggcaggagaatcacttgaacccag
gaggtggaggttgcagtgagccacgatcgcatcactgcactccagcctggcaacagagtgagactccatc
tcaaaaaaagaaagaaaaaaatagtgaagtgaatttgtatgcttttggctgatatggcaacttttttttt
ttttttttttttcttgagatggagtctcactctgtcgcccaggctggagtgcagttgcacgatctcagct
cactgcaacctccacctcccgggttcaagcgattcttctgcctcagcctccgcagtaaccagccaccacg
cccagcctgatatgacaacttttaagaactctccagtgttcacttggactgccaagccccatttgaaatc
tctgttagacaattcaacgtgaaatttaagagtaattctagattttcatactttgaaaaatactccagta
aagcctggtaagcaagtggccctattgttttataattttatagtgcagaaacaggctttttgctcatcaa
aagtatatagtctgggctgggcacggtggctcacacctgtaatcctggcactttgggcgccgaggcagtg
gatcacctgaggtcgggagttcgagaccagcctgaccaatatggagaaaccccatctgtactaaaagtat
aaaattagctggatgtggtggcgcatgcctgtaatcccaggtactcgggaggctgaggcaggataattgc
ttgaacccgggagacggaggttgcagtgagccgagatcgtgccattgcactccagcctgggcaacaagag
tgaaactccgtctctaaataaataaatatatagtctggaatatatgtttaattctaatgccaccagtcaa
cactacttaaagacttatccagatttcagtcatgcaacaaattttgagcacctgctgtgtgctggctatg
gctggtgtacactacagagcaggactgctgctgtgggtttacattctaccagtgggtccagatgagccgg
cagaagcaaactggcaagtataaaacaaatacaagcatggttgagggggtgtggtgggtagtgacctgat
gttgagagagcctttcaaaaagcatgcagcaagcttgtgaggggagaggagatggatgagagaagctttt
ctccagggtgaagtgtggtatctgagcagaggcctgaagtctgagcagttgtatgataactgatggaaga
tggttttggcagtttctatctttatataacttttttctttctgctaattttagggaatttaagaagcttt
cagaccatgtttgttgatgtttttcataagcaccattctttaaatacaaatctttattttctctggaaaa
gctgcatctaatcttaatattaagagtaatttttaaggccaggtgcagtggctcatgcctgtaatcccgg
cacttggggggccagagcaagaggatcacttgagcctggaaggttgaggctgcagtaagccatgatcatg
ccactgcactccagcctgggcaccaggcttcttcaaaaaagaagaatttttaaattgttgttagtattgc
ttttgctttttactgatctctgaatgtccccacccagaggacaaacagaaacagaataaaggagttaaaa
caaatataaactatgttcaaaggtgacagtggagagcgtccaaagccaaaaaaaaaaattgttagcaaca
ttgatttcatctgatggcattgactaactgcaatagcattcataaaacaaagcttgccattatatggtat
gagctatttatcgcagaaatactctcctagaagacttagagtcagctattcattcataagtgtgggactc
tgctgaaaaaaattcacgttgaaaccagaagcttctaggatttccttcctagccaaaaataaagcttcct
gctgggcagtctgcccaagtttttggttaacttatggtgaaatcactaatgattggcattttctggccaa
tgaagtggggatcccatctttcaccgggagtatttcctgagttctaactctgtatgtgtgtctagagaga
aaaaaaattacttacgaaaaaaacatttttgttgctcagttgctgttttattaaatatgcatctgtttca
tatctttcctcgaagactattattactatgcttttattgcttcttgttgtataatatttttatcagaacg
agacttagttacatattttggggtccaaaagtgaactttataactgtaaaattaaggtacatttagcagt
tccgtgctactttatctgtatctaatgactatccagtctgttacaactggaaccaaaacaaaatttcttt
cccacaactacctacgtgctgacaagtgagttctgttgcaaagtttgtactttgatagaagaaccctaag
tgctctggtggtgtggatgccccatgctgtgcaatgatttctttctcccattattatcttatctgttcaa
attattagggaacgtgctttcttacagtatagcacatgactgacatcctttgccctatatagacatgagc
ataaacacaaatgcaggcccagaaaatgcatggcaaaataagtacaatgaaccaacattttcttaatgaa
atgaaaacaacactaacattttttcataggtaaattaaatcagagatcctcaggaggagatacccatggt
cctctggaaaacactgatgggggagggttctgagtatttccaatcctgtttcttttcttttcttttcttt
ccttttcttttttttttttgagacggagtctcgctgtgttgccaggctggagtgcagtggcgcgatcttg
gctcactgcaacctccgcctcccgggtgcaggcgattctcctgagtagctgggattacaggtgcacgcca
tgttggcgaggatggtctggatctcttgacctcatcatgatctgcccgcctcaccctcccaaagtgctgg
gattacaagcgtgagccattgcatctggcctgcagtcctgtttctaacagcattcgcacagacaggatgg
ctccttttttggaatactataaaagaagtcttacttggtatcgaagatgcaattaagtcttgtctaagat
tttaagattctaattagacattaaaaccatgtttattataagtgtcctgaaaatgaagccactcagaaag
ctagttaaaccatgtttttgcccctttatctaaatctcgtattgtattttgtattttggcttcattggac
taattgcgatttttctcttgcagtgagagggtccaagcctggtaagaatgtccagcttcaggagaatgaa
atcagaggactgtgcttaaagtctcgtgaaatctttctcagtcagcctatcctactagaacttgaagcac
cactcaaaatatgtggtaagtttggggcacacaggttatcttttgttacatgattgattaactgagtcaa
ggatatgttgatgaccttgtattagctagcagaatgattatcctgtttgcgaatagcttggaactactta
aaagtaagttaaatagtagctttagaataaatacctctaatggacttagtttacttagatgtacttcaga
tttttaaaaaaaatattttgctgaaggaataaacattgcaaaagaaaaaaatgaaactaagaatggagag
ctgaggaaaggtctgaactacatggatgtataggaatatgtttaagtttaaagcaacactggttatagct
aaaaatagaacttccttgatactgattttgtggttctgttttctgcataatagaagcaaataatctgtgt
tttaactcaatgtaaaaccaaactttattcgtttgagtaatatattcttatagcatatttgcattgataa
aactttgtgttcatgtttttaaaagtaaaatgttctttggcattatctagcagggagaaattgaaagcta
aatgcattgatgggtggggggtgggggtggtgtgtatgtgtgtatgtgcttttgttcagatggggccaag
actcactagaagtgcagcagttctgctgacacagtacccagagactgagctgtcagagatctgatattga
tcagtcagcctgaaagggataccatgttacttggcaagaatgcatgcttggacataataaccctttccac
agaataaattcttaatagttgaattttatgtgaggttctggacatacacatatacttaaaatttttgtta
agctcagttgaactaaatatgatataagattggaaattacttagtgttggctgcccagttaaactctcat
tactagaggtcaatgccattgtacaatgaatatttaaagaatgtctttccaaagctttgttagtggattg
agctttaagtatgggaacactgtgtaacaaaattcttgcttccaggtgacatccatggacaatactatga
tttgctgcgactttttgagtacggtggtttcccaccagaaagcaactacctgtttcttggggactatgtg
gacaggggaaagcagtcattggagacgatctgcctcttactggcctacaaaataaaatatcctgagaatt
tttttcttctcagagggaaccatgaatgtgccagcatcaacagaatttatggattttatgatgaatgtaa
gtacttttctatatctaaggaagagttattgaaattaagatatttatcataaacaattatcacatacttc
tgttaggaattgtgagagtttagatgcagtggtatcatctctcatgttttctatttcatcaacttgtttt
tagcttagcttttttaggggggtgggggaacggagtctcactctgttgcccaggctggagtgcagtagca
ccatcttacctcattgcaacctctgcctcccaggctcaagcaatcctcctgcctcagcctcccaggtagc
taggactgcagacatgcatcaccacgcttggctaatttgtgtatttttttgaagagatggggtttcacct
tgttgcccaagctgatctgaaactcctggctcaaggaatctgcccgcctaggccactcaaagtactggga
ttactggcgtgagccatgatgcccagccagttttttctctcttgggtagaaaaataacactgccgctgta
ataaaataaggtcttggattcttttatgatgtgatgatcagattaggaagtaatgatcatggtaatggtt
gctttaagatttaaattgttgtattcacagataacttaattcttagtagaaatctgagaaccattccaaa
tagtttttttaagcaactttattatgtaatttcattccataaaattattctgttaaaagtgtacaattca
gcgatttgtagtaaatttatagagttatgcgaccatcaccatagtccccaggcaaccactaatctacttt
ttgtcttttctagagaatattgcatgcaaatggaatcttgcaatacgtaaataattagtttttgaaatta
ttatagtttttctccttctggcatatccacacccaggaagaatggcaataactttcctaaaactaaaatg
tgctgaacaaagattatttttacagtattttacacattttgtgacccattttggattgtttaattaaagg
tatacttcgatttgactattgacaaaacacagttcttaaagctttgaaaataggctgggcacagtggctc
acgcctgtaatcccagcactttgggaggccacagcgggtggatcacaaggtcaagagatcaagaccgtcc
tggccaacatgaggaaacctcatctctactaaaaatacaaaaattagccaggcgtggtggcagatgcctg
tagtcccagctactcaggaggctcaggcaggagaatcacttgaacccaggagtcggaggttgcagtgagc
caaaatcacaccacagtagtccagcctgggtggcagagtgagactccgtctcaaaaaaaaaaaaaaaaaa
aagctttgaaaatatcagttcagtaaagggtttcaagtcagaaaaggttttgaatctttaccttcacctt
ccttttgtgctcatcattgctgccaggacgggtttcaggtttggagtgaagaggtggttaaggaacttca
ggtacttggcatttgctaatatgattatagggatatcagcaaagggttgtccctgtggccctcccctaga
ccacatcgtaacctagagtctccagatgcagtgtttttccatctcaattcacatagatatgttgagctag
cacatttgttagaagtgcatcctagaaagattatattcacaaactgagcccttctactaccaccctttcc
cacaactggatgtttgcttggtctgtgaccttgagatccagctgataagatggggtggcaggaatcatca
gggacaaaaattctatgggttctggactgactgatagcattgaacttgtacatgaaaggtattcaatatt
aacagtgattctgtgtcagagaagtaccagtaagccagttactactccttaggtagtgaaatgcaaggca
ggtgtgatgggttttttttgtttgtttttgagacagagccttaggctggagtgcagaggtgggatcatag
cttactaccacctgtaatttctgggtttaagcaatcctttcacctcagcctccctaagtgctgggattac
agtcatgagccaccacacccagccaacactgtgttttggatacttattccaagtgttactcttgagtttt
ttgttttttgtttttttttttttttttgagacggagtctcgctctgtcgcccaggctggagtgcagggcg
cgatctcgcctcactgcaagctccgcctcctgggttcacgccattctcctgcctcagcctcccgagtagc
tgggactacaggcgcccgccactacgcccggctaattttttgtatttttagtagaggcggggtttcactg
tgttagccaggatggtctcgatctcctgacctcatgatccgcccgcctctgcttcccaaagtgctgggat
tacaggcgtgagccaccgtgcctggcctgctcttgagttaaaggattccttttacatagctgttagccca
gcaaatacttttcatgtttgcactgatgaattcaaatccagttgacctcatttttgtgacgtgtatccta
atgtaatgatactggctgcatcagtttcggtttctacttaatgcagaggcctttaaaaaatgacttagaa
gatcagtgttggttggtatcagtttcgactcctgtttggtcatcaacacaatttgtagatcttatgctca
agcttcctctctctcatcaggcagatataatttgccatccagtctacttgtgggcatgaaagtgcccttg
acataggtggcttttggttgaaggcttgcagacatttaatatatatataaaatacttaactaattttatt
aaaatgaaaagttgtattcaaatgttgattttcatgggacaatttaaaaaccactgcctgttttccaggt
acataagtactttatactaccatttcttttttttttttttttaactttgtaaatgtgatggaaaggctga
ggtatatgttgttgggtaaaacatgagtgtttgttgatctgtttctgctgccgtagctgtctgatgtaca
gtaatgcgaaaccgcattacaggttatggctcagaatcttaggatttggaagctaggcgattaccctggc
taaacctttcaggagccagatgttcctagtggtctgaggtagccttagggaggaaatgaagagagaatag
aaagaggcaggcccacgcttaaagtgctgagagggaaaaggaggaaacaaactgatttttgatggagaag
ccagcaggaatcagcaggagaagctggagtatgtggcataactaaagaatcaaagccggggcgtggtagc
tcacgcctgtaatcccagcactttgggaggccaaggtgggtggatcacttgaggtcaggagttcaacctg
atcacaagaggtcaggaggtctgaccaacatggggaaaacccatctctattaaaaatacaaaaattagct
gtggtggtgcacacctgtagtcgcggacactcaggagattgaggcaggaaaatcactggaacctgggaga
ccatggttgcagtgagccaagattgcgtcgcttcattccccctaggtgatgcagtgagaccccacctcaa
aaaaagtatccagaagtgaagctgaccttggggaaagaggaagtcactgtcactggggagctaagacagc
agctctgtttttataatacaggttaagtattccttaccgggaatgcttggcactagaagtgttgcaggtt
ttttgtttttgtttttaattttggaatatttgtgttataattactggttaaacatccctaatccaaaaat
ctgaaatccagaatgctccagtgagcatttcctttgagcatcatgtgagtgctcaaaaagttccaaattt
tggagcatcttggattaaggatactcatcctgtggtggtagtagtagtagtattgctgttggaaatggga
gttggtggtttttggagattgcccttcagcctttcgtaagaaggagtttgcttttttttgagacggagtt
tcactcttgttgcccatgctggagtgcaatggtgcgatcttggcttactgcaacctctgcctcccaggtt
caagcaattttccttcctcagcctggcgggagaagtttgttaaactttccgaaccccctagtgaaggacc
tctaaagaaatatgcaacagggtgaaccatagtcagtgttagacccagtcattgagaaaccagaaattat
taggagaaaagataacactcctctgcagagagaaggcaaacatttgttgaatttacatttcatttcaagc
cctgtggtaaactccctaagctgcttgatttgcattacagggtgggaggtgtggttcaggtcagttgctt
gggctggctctaactctcaaaggagaggtataggtttaaacctttagatgtccctgtattccccaggaaa
agagactaaggacaaactcttggtttgttttgtttgttttgttttgttttttgagacggagtttcgctct
tgttgcccaagccggagtgcaatggcacaatcaaggctcaccgcaacctccgcctcccaggttcaagtga
ctcttctgccttagcctcccaagtagctgggattacaggcatgcgccaccacgcccagctaatttttata
tttttagtagagacgggatttcttcatgttggtcaggctggtctcaaactcccaacctcagatggtctgc
ccgcctcggcctcccaaagtgctgggattacaggtgtgagccaccgcgcctggccaaactcctggttttt
aaagattgtgcttagtatagtaagtacaggaaacaaaggcatctggacaattggacaatcctcaaccaat
ccagagaaatttgttcttgcaaggtatgtgactaggttttaaactcagaacatcagtttcctgtagaaac
agtgcatagaggcatatttctgtcagaactgcactctgactccagaatgtgtggatgcctgtgctgttga
gggaacatgaccatcctgtgacatcaggcctgtgggagcatgtggagtctgtgcagtgttggaaacaaat
gccctgtagtgcctgtgagaggcagccactgccgtgtgatcattgtgcctcctgctagagtactgagggc
tggggatcctctggtgtcagtaggttattgaggggcatatatgggggatgggtgctaactctcaggtttc
ctcagaaggaaggtcataacgccacatgtgtaagttcgtaattgaattcagaagtaggggactagctgcg
gtgagattggacagagcagacaatttcaaacagaattgatgctcccagataattcctgggatttgatggc
agtccttaatcattatcaaatctggaattctggtttcatgcttctgtggaatttaaaaagatttcatgtg
aaaatgtaagtcattgcttttagatcagatatgtgtctctcctaaaccccttccctctaggagagtcttt
ctttaaagagatggggtctcactatgttgcccaagatggtcttgaactcctgggctcaagccatcctcct
acctcagcctcccgaagtgctgggattacaggtctgacccaccacgcccagctcttattttaaaagagcc
ttaaaataagatggttttattaagattcttaataaaaacatagtttcatttgcttaaaaggcacccagaa
tcttgctttgggggaagttaatcctgagagaatgagcctacggaacagtaatactctctttttaatgtaa
tactctctttttaatcacaggtaaaagaagatacaacattaaactatggaaaactttcacagactgtttt
aactgtttaccgatagcagccatcgtggatgagaagatattctgctgtcatggaggtaggtgggctgatt
tttgttccaaataggttttgatcccttccaaagggtgaacactttaacatttgtgagttatcattgggat
gactttctctgacagaacactctcctttgttgatgtgttgcattgcactttcactctgatacagccagga
tcatttggataaaaatcttaaggaattgatttattacagggaattctagactctgtaagctaggtttgaa
aatagagcagaaaatttttatttttattttattttttgtaaggatgagttctcaccacgttgcccagtct
ggtcacaaattcctgggctcaagcagtcctcctgcctcggacttccaaagtgctgggttacaggtgtaag
ccactgtgcccggcttagaaaagtttttggttttttttgtttttgtttttgttttttgagacagagtctc
gctctctcacccaggctggagcgcagtggcaccatctcggctcactgcaagctctgcctcccaggttcac
gccattctcctgcctcagcctcctgagtagctgggactgcagccacccgccaccacgcttggctaatttt
ttgtatttttagtagagacggggcttcaccgtgttagccaggatggtctcaatctcctgacctcgtgatc
tgcccgcctcggcctcccaaagtgctgggattacagacttgagccaccacgcccggcagaagagtgtttt
taactataaaatgaacaatgtgtaatttgttgtgccagagatcctctagaacactcaagatgtgagtaga
ttttcccttgattgttttttatcctctcagaactttctctgacttggattttaaaagttattttggccag
gcatagtggctcgcatctgtaattccagcactttggagaggccaagccaggaggattgcttgatgccatg
aatatgagaacagcctataacatatcgagacccctgtcattagaaaaagttgtaaaagttagccaggtgt
aatggtgcacacctgtagccgtagctatttgggaggctgaggcgggaggatcgcttgaacccaggggttt
aaggctgcagtgagctatgatcgcaccactgcactccagcctaggcaacagagcaagaccctatctctga
aaatatatacatatatatgttataagctgttttgctttgctcagtggttgtgctttctataatgtaatta
aaatagtaattgcaaagagaatttcttttcctgtatggtctgaaattaatcagccatcaaaaagcctgca
taatgtcttttatctctgtgcttctgtacttaggttttggtagagttggatacatggtttttaactctta
caatatgcatcccatattgtaactcgatacaataagctcttgtagattttagccatcaaaaagcctgcat
aatgtcttttatctctgtgcttctgtacttaggttttggtagagttggatacatggtttttaactcttac
aatatgcatcccatattgtaactcgatacaataagctcttgtagattttaatggtctgtgaacagtgcag
gtttcctagagaactacctcacactgagcatccttatcttctcagctcagtcattataagccatcaatca
taacatcgctcattattgtcatcaactgggggaagcccatagaagagttttttaaatcactggattttat
tttacagtgttctgtataatataattgtttccatgtactgtttaaacctactgcaccccatgcattactg
catgtaattctagtgtgtatccagttgtcctttttctgtgttttttttctttaaccaaaagtaaataaaa
tataatcatagatttcagtttttatgtcaggattattgaatttctctgtagacaaatggaacttttgaag
gggtagtatccttttaaaagttctgctttcttcattttttttttttcctcattgaataggtttatcacca
gatcttcaatctatggagcagattcggcgaattatgcgaccaactgatgtaccagatcaaggtcttcttt
gtgatcttttgtggtctgaccccgataaagatgtcttaggctggggtgaaaatgacagaggagtgtcctt
cacatttggtgcagaagtggttgcaaaatttctccataagcatgatttggatcttatatgtagagcccat
caggtattgattttgaattttacatgtacacttaagagcatgtgtgtgagcacagattctccttgttgat
tttggtgggtaggtattgcttatttatacaaagtgtttctggaacacaagatatcaggctcaatgggaaa
ctgaaacatggttttactcacctaatatttgcacctatttatattgaaaaaattttctcttcaaggtggt
tgaagatggatatgaattttttgcaaagaggcagttggtcactctgttttctgcgcccaattattgcgga
gagtttgacaatgcaggtgccatgatgagtgtggatgaaacactaatgtgttcttttcaggtagagcatg
ctttcaacatgatttccttttaatatgtttacactttgctgattgaaaaatataattgctactttttgcc
tagtgttcacttatgctgagtaggctaagtttttattttcccttaaccagtgtgtattatagttgaataa
tgtgtggatcccttgtaaaagaaaaaattaattatggcaccttttcccagtgttgaattataaatttttg
cattgcatattggctccttaatgcttatggctcttttactgtaaatctaaaaccttaatgacaatgtaat
gttcagctaaaattaaatcaccacttgaatgtgaaaagtactgagtgccttgtgctatgtgtttacttag
ttctgccctctagtctaacttccacaaaacttctgttaatgtaccatgacatcatttggccttcagttgg
cacaaaagcattatttagaatctgcctcagcctttttctcatttgcttattatgatacttgtacaacttc
aagccagcctagtcataaacataaatacagccaccaaatcatgtggcactcatcagttaaatgaattaaa
atagttttttaccaattatcagagagaacttgcagctttcaaaattccaaagattgtgcaaggatcccct
acaggttggagaaacacttttgtaaagaactttttcccctgtatcagaaggagtagctaaagatactacc
aggtgtagacttcagaagattgtcattatactcagaggcaaaataatttgtccagataatagattttatc
tgttcgtcatagcgttaattaccaaatgtttacattcaacatagcttgtgttaatatgctaggggggctt
ttacagttgttggggcagttgtgaacaccatctgaacagaagatctgaatgaaatttaaggactcttaag
atttggggttgagtatatttcctaaattaactgtcttttgaatctccagattttaaagcctgcagagaaa
aagaagccaaatgccacgagacctgtaacgcctccaaggggtatgatcacaaagcaagcaaagaaataga
tgtcgttttgacactgcctagtcgggacttgtaacatagagtatataaccttcatttttaagactgtaat
gtgtactggtcagcttgctcagatagatctgtgtttgtgggggcccttccttccatttttgatttagtga
atggcatttgctggttataacagcaaatgaaagactcttcactccaaaaagaaaagtgttttgtttttta
attctctgttccttttgcaaacaattttaatgatggtgttaaagctgtacaccccaggacagtttatcct
gtctgaggagtaagtgtacaattgatcttttttaattcagtacaacccataatcatgtaaatgctcattt
tctttaggacataaagagagccctagggtgctctgaatctgtacatgttcttgtcataaaatgcatactg
ttgatacaaaccactgtgaacattttttatttgagaattttgtttcaaagggattgctttttcctctcat
tgtcttgttatgtacaaactagtttttatagctatcaacattaggagtaactttcaaccttgccagcatc
actggtatgatgtatatttaattaaagcacacttttccccgaccgtatacttaaaatgacaaagccattc
ttttaaatatttgtgactctttcctaaagccaaagtttctgttgaattatgttttgacacacccctaagt
acaaggtggtatggttgtatacacatgctgccttcttggggattcaaaaacaggtttttgattttgaata
gcaattagtgatatagtgctgtttaagctactaacgataaaaggtaataacattttatacaatttccata
tagtctattcattaagtaatctttttacagttgcatcaggcctgaacccgtccattcagaaagcttcaaa
ttatagaaacaatactgttctatacgagtgaccgattatgctttctttggcctacattctttattctgcg
gtgaagttgaggcttataagttaaaacaaaggaactaacttactgtccaccagtttatacagaactcaca
gtacctatgacttttttaaactaagatctgttaaaaaagaaatctgtttcaacagatgaccgtgtacaat
accgtgtggtgaaaatgaattcagacttattaaatgatgaacttgttaaatcttctcagtgtctatttat
cagcacaatacacacaggagaactgttgatggcatattgaatagattttcctgaataaattgctctggaa
accacacggttgcattttggttttgcttctttcaaatagtctgttttccaaagatggattataatacaga
tactaatactctttgagttgtaacctggagacatctatttgtgtaatagagtgatgaggtggaacgatcc
tcctctaccatttggggagatggggattaatctgtgtccatagttttcttactgtctgggttcacttgaa
cttagtggcgaatttgaaagcagctcactttgttttctgagtcagccacctatattttcatgcacagttc
tttgatctcttcccccttccctggtttatatggaacacagtatcatgtcatattttgtcacaagtatgta
tcttaatttctcaaaggtacgctaaaaatgagtgaagagcatgtcatttgaaaagctttttttcctgtta
caaagtggtctccctgagggcgttggcagaggacctagcgaggaactctttgaaggacacagaagagcac
agggccatggactcccctctgctaacaagccccggtcctgaggcatctggaccctgccatgctcccatca
aagagggaagggggacctcattacctgcctgctccacattgacaatatccaaacttgtgctgtttcactt
ttacgtaatattctcccagacttctttgactataggaagaaaacagtgaatgataattgtgtcaaaggca
aattctgcacatctttatgtcctgaatgttaatgaattgatactaggatttcaaaatgctataaagtaaa
attgggctgctctactaggattatatcattattggagtgtggtatcaggctcaagtttttcttaaggaac
atttatcaagtactaaggattatatcattattggagtgtggtatctggctcaaatttttcttaaggaaca
ttggtcaagtagaagacaagattaatctttggttttaggacctgagatcattaggagagactcagtttga
aatctgtttgacagcagaacttgagatgtgtgtgataacccaagtcttggcccctcctacctgatttgag
tgagtgacctgaatgctgagccactgtgttcctagctatgaaacgtccatagcagaagccgtctcacatt
gtgaatcttgagtgaagttgtgtggtgtctttagaatgctcaactcggtggtatttgctaaatgtttgct
tttggttatcttatcgatcccagttctttcgtgtctttgcacatctcggttggctgagtttgaagtgtca
cttgcctttgctttgctttgcacctgataccaagccatggatagctaagtatgtctgagcatttactgca
taagggaacaccatcatggatgttttcagcatatgcaaaagaagtaactaggaaagaggaagttgtaatt
attagtgtgatactattctattaagtgttttggtttggttttgtttttgtcaagtattctgggtcacaag
atgacagcaccattacaaaaccatacagaaaggcaggtgctaggcatccttagtcgctcaatcatctatg
taataccctcccatagaaattgctgatttggtacacaaactaggcctgcccatgtctagttgcagtggac
agcacagatgctcacatataacccttgttcttgaagtaccttgatttaaggccagagttctcgagtaaca
tacacataaatgggatatggaacacagggactaggaaaaaattactgaatgctgagccatgaagaaggca
catgatttaaactgatggaaaggaatatcaatcaaatttctcaatgtgggtccatgatgagcgtttgaaa
gggaattcagtttgctgggaagtgaagatacaagctaccaagttaaggcatcatgataccaaaaaacaca
aaggaaccattgcagggaagtgctgcatgaaaaatcttaggtttattttggcagtttgcagacaccatgt
gaattgagacacggagacaagggccatgtaatccaagtatcacctgaaattagtagaagggacaaagctg
aggatctgcatcagaacaaagaaggaatgtcttcctcatgtttgggtctatagaagacattaaagaaaac
ttccagaaagtgggtttgaggccgggcatggtggctcacacctgtaatcccagcactttgggaggctgag
gcaggcggattacaaggtcaggagatcgagaccatcctggctaacacggtgaaaccctgtctctactaaa
aaaaatagaaaaaattagccgggcatggtgatgggcgcatgtagtcccagttactcaggagcctgaggca
ggagaatggcgtgaacctgggaggtggagcttgcagtgagccgagatcacgccactgcactccagcctgg
gcgacagagcgagactcttaaacaaacaaagtgggtttgaagtgctatgtctccattttcctatcttgag
gaaaagacccaaaactcaacctcatgtaccagctggcttgaatctgcaggggtggttttgatgagtaaga
actttcagtcctgcaaatcagatgtatgcatcagcccagactggaagaacatggcaccctctgccccagg
ctttggtttccttctcacccacagaagaggaataactagaatacttaacgatgggattgtgaggattagc
ccaccaaaaatctacaacttgccttagtcataagcattcaggaaacagccctggtataagaccaatgaat
agccctaagatttagaccaatggcaacacacaggaactttgattaggaaaacttgggccaggcacaggca
gaagtcacttttcattcatccttgccaacgtgtgagcttcagaagagtggaaatgatggactatcaacac
cttcagcctacgactcaaatagaattgagtacttgcattagtgcatccctgaattagtgggtggttcata
gttgtgtgatcacagagatgactgccatcccatctgatcagctgtagctccaacggacttgttgtcatcc
agatgtattacaagtattccaataatttcaaacacaggtaagtatttcagttgtgagaattatgtcagtt
atttgaaagataaatatgttagaggattaaagcacttcagttaatcatgtaaacagttttgtaatcaaaa
agttaaaactaggctgggtgcagtggctcatgcctgtaattccagcgtgattgggaggccgaggtgagtg
gatcacttgaggtcacgagtatgagaccagcctggccaacatggtgaaatcctgtctccattgaaaatac
aaaaatcagccaggtgtggtggcacatgcctgtaatcccagctactcgggaggctgaggcaggagaatcg
cttgaacctgggaagcagaggttgcagtgagccaagactctgccactgcactccagactgggcaacagag
cgagactctatctcaaaaaaaaaaaaaagttaaaactaagaaagtttgcagtaatagggaaatagaattt
tacctacataagttcaagaactttatactcttgaactaacaaaagtttgtaagaattttaaaatataagg
catgcaaaatatcaggaatgaatcagtacggcaagtcttttaagtgcatctcagaaagcatcctgaggca
ctgcccaaagcttgagattgaggggttgtgtaaaggcactggcatcaaccagattttttaagtctaggaa
ttgagaaggaagacataagtctttaccttgctattgggatggaacaaccaatttggtggtaggaccagcc
aaaaagcaacttgcctttccaccaacctcaaagtgagcatctatttagtattacaaacatgcagataccg
caggagcaataaagcgacaggtggtgctaagaaggtagcatactggccgggcacggtggctcacgcctgt
aatcccagcactttgggaggccaaggcaggcagatcacctgaggtcaggagttcaagaccagactgggca
acatggagaaatcccatctctaataaaaatacaaaattagcctggcatggtggtgacacatgcctgtaat
cccagctactcaggaggctgaggcaggagaatcgcttgaacccaggaggcagaggttgcagtgagctgag
atcgcaccattgcactacagcctgggcaacgagcaaaactccgtctcaaaaaagaaaaaaagtagcatac
cagcccagcatcaaacttgggagaattctgatgattgtaataattaaatggtatcaattaaatttttttc
cagtatgggcaacatggtgacaccccgtctctattaaaaatataataatacaaaaaattagccaggcatg
gtggtgcacatttgtggtcccagctacttgggaggctaaggtgggaggatcgcttgagcctgggaggcgg
aggttgcaatgacctgagatcgcgccactgcactccagcctgggcgacagagtgagaccctgtctcaaaa
aataatttttgatagaatttcaaattcattcttgtgttccaaacaatcagtttcctaatagcgatattta
gctatgaccaccaaatactgattttaatactcattcagccaaaatgcatatttggtgcaactcttgaaac
aattctaaaatgtcacttaaaagtgcgtattccttttttttttctagccacccaattagcacagtatcaa
acaccatgtctcccagagaggaccatcccacacaccagttgttcagggctggtgaacaagagaacagtaa
actggtcgttatattcttggacagactggcttgacctttccctggcaagttaggtaggtgtggcagaggg
agcgggtgggataaagggtgtttttgctgaggttggcagaagatttttaccatctgatggggctttgctc
attcaccagctccacaagcccctcaaggagtcctgttccatacctgctgttagtttaccataatgaaatc
acttcccaattttttttttttttttttttttttttttttgagacagagtcttgctctgtcgcccaggctg
gagtgcagtagtaccatctcggctcattgcaagctccacctcccgggctcaagtgattctcctgcctcag
cctcccaagtagctgggattacaggtgcgtgccacacacccagctaatttttatatttttagcagagacg
gggttttactacgttggccaggctggtctcaaactcctgacctcaagtgattcgcctgcctcagcctccc
aaagtgctgggattacaggccttcccaagttttgattactgacctacaaacaccacatatcttctctagt
gcttgcccttcagattgagaggtggaatggacagatcgtagactcacatggtcctccgctcaggaggctg
tccagcataagtgaaggacccagattcagcgtggatcacagggttagctggattttactcactagcttgg
cttagattggtttcagatctgccccatgttcctctcactgttctagggccattgggtattacttaggagg
gtggactgaggagaggagagcaagcaggcaatcttaagatttgctgcagtgcaaattactcattgggtgc
acattttggttgccatgaatgctgtgtttttatacttttatttttagaagccataatcttgctgaggtca
atataatcttgctagaatgaaagtttttttgttttcgtttgtttttgagttgtagtcttgctctggcacc
caggctggagtgcagtggcacaatcttggctcactgcaacctccacctcccaagttcaaacgattctccc
acctcagcctccagagtagctggaactacaggtgcccgccaccacgccgagctaatttttgtacttttag
tagagacaggatttcaccatgttggccaggctggtctcaaacccctggacctcaggtgatgcgcctgcat
tggcctcccaaagtgtgagccactgcgcctggcacaagaatgaaagttttgagaaagtgatctgcattct
ccactccagaaaaccagaattcctcttaagacaatgctggtcgaacttttttgacaggtggaacccatca
aacaaaataatattggaggcctcatgtaaaatgaataaaatcagaacagctctgttaaaagcaggggctg
ggggctgcttgtccccgaacagcttccacctccaaagattgaaaccacgctctaggaaaaatattggctg
ctacatacaggcctgtgtttgtagaccagcattagtaagatgttagtacaacttattaggtagagccttc
cctgattttgtgatgccttgtctaagatccaaatagctaagaagttcaatccaagtattctccaagtaga
aagtcaacacatggccaggcacggtggctcacgcctgtaatcccaacactttgtgaggccgaggtgggca
gattgcctgagctcgagagttcaaaaccagcctgagcaacacggtgaaactcatctctactaaaatgcaa
aataattagccgggcgtggcagcgtgtgcctgcagtccaagctactcaggaggctgaggcaggagaattg
cttgaacccaggaggtggtggttgcagtgagccaagatcatgccactgctttccagcctggacaacagag
tgacactacatctccaaaaaaaaaagaaagtcaacatgtaactaccattttccaagttatttgctggttt
tcatttcttttttctttttttgagatggagtttcgttcttgttgcccaggctggaatgcaatggtgcaat
ctcggctcaccgcaacctccacctcccaagttcaagcaattctcctgcttcagcctcccaagtagctggg
attataggcatatgccaccacgcctggctaattttgtatttttgtagtagagatggggtttctccatgtt
ggccaggctggtctcgaactccctacctcaggtgttccacccgccttgccctcacaaagtgctgggatta
caggtgtgagccaccacgcccagcctggttttcatttcttagcactgtggtaacagacacaggagatcag
aaaagcattttgcagatttgaacaaagcatcctttaggagaccagggtttttcatcaaggggatttcagt
gccccccctttttttttttttttttttttttttttttttgagacagtttctctgtcgcccaggctggagt
gcactagtgctaacttggctcagtgcaacctccgcttcccggattcaagtgattctcctgcctcagcctc
ccgagtacctggaattacaggcacccaccaccacacgtggctaatttttgtatttttagttgagacgggt
tttcaccacatttgccaggctggtctcgaactcctgacctcaagccatccacccaccttggcctcccgaa
gtgctgagatgacatgcatgaaccaccacgcccggcctgtgtgtctgtttttcaatgtgtgtgtatctat
aaggtatattgtcattttatatgttattttatataatatatatggtcccgtgtcacttaacgatgggaat
acattgtgagaagtatgtcattaggcaatttcatcattgtgcaaatcaccatagtgtccccacacaaacc
tagatgacataagctatacggtagagcctattgctcccaggctacaaacgtgcactggaccctgtaggca
attataacacaatggtaagtatttctgtatctaaacatatataaatatatagaaaagctacagtaaaaat
atggtattatagtcttatgggaccaccatcacatatgcggtccatcattgactgaaaactcgttaggtgg
catgtgactgtatttttaatggtttgttgttgttttgttgttgttgtttgagacagagtttcattcttgt
tgcccaggctggagtgcaatggtgcgatctcggctcactgcaacctccgtcccgcaagttcaagcaattc
tcctgcctcggcctcccaagtagctgggaccacatgcatgcaccaccacgcccagctaactttgtatttt
tagtacagatggggttttttccatgttggtcaggctggtctcgaactcccaacctcaggtgatcctcctg
cctcggcctcccaaagtgttgggattacaggcatgagccactgcccctggctagtgtattttttaaatga
aatgtcttaatacaaaagaatacataaaacacccacatacccatcgcctgggttaagaagtagaatatta
ccaataacttaagagcccgtgtgcgtacctccccagttacatacttacccatctccctcgagagttaacc
atgatcttgaatttgaaagtaatcacttccttacttttcttcattgtgtttgtatccctaagcaatatac
tgttaaattttgccaatttatttatttttatagaaacagggtcatgctatattgcttaggctggtctgga
actcctggcctcaagagatcttcctgcctcgggctcccaagtagctgggattataggtgcaaaccacagt
accaggcaatttttgagcttcatagaaagagaaatatatgtatttctgtaaacagtttccttcagcattt
aaaaatttttatttatttatttattttattttgagacagagtctcactctgtcgcccaggctggaatgca
gtggcaccatcactcactgcaacctccgcctcctgagttcaagcaattctcctgcctcagcctcctgagc
agctgggattacaggcacatgccaccatgcctggctaatttttgtatttttagtagagacggggtttcac
catgttgtccaggctggtctcgaactcctggcttcaagtgatccacctgcctcagcctccttttaaagtg
ctgggattacagacgtcagccatcctgcccggcctaaacatttttatttatattttagagagacagggtc
ttgccttgttgccgaggctggtcttgaatttctgagctcaagtgatccgcccaccttagcctcccaaagt
gctaggattacaggcatgagccacctcacccagcaagcattttttttttttttttttttttttttttgag
acagagtctcattctgtcacccaggctggagtgcagtggtgcaatctccgctcactgcaagctctgcccc
ctgggttcatgccattctcctgcctcagcctcctgagtagctgggactacaggcacccaccaccacaccc
agctaattttttgtatttttagtagagatggggtttcaccatgttagccaggatggtctcaatcttctga
ccttgtgatccacccgccttggcctcccaaagtgctgggattacaggtgtgagccaccgcacccagccta
atttttgtttttcaaaacaaaaatatttttgttttgaaaatttttgaaatgtttaatagcgacagggttt
caccatgttggccaggctgggctcaaactcctgacctcaggtgatccgcccaccttggcctcccaaagtg
ctgggattacaggcctgagccactgcacctggccttaatttgcattattaatattagaggttatctttgc
cctttttcctattctctttgggaaatataatgaggtgtattagacctcattctgtctttcatttcacttt
aacatcttgtttttttccatctctttccctatggtttgcattcttgataatgtcctcagctctatcttct
agtttactaattttttcttcagttgtgcttaatctaatccatgcactgaatttttaattttaattactac
atttttatctcaagttctattgggttttttgttttcctttattattattattattattattattattatt
attattattattttggagttttttgtttttcacaatccacttggtcattcttcattatctattatttctt
gctaatctttttaaaacttttaaaatttgaccaggcgcagtagctcatgcctgtaatcccagcactttgg
gaggctgagtcgggtggatcacttgaggtcaggagttcaagaccagcctagccaacatagtgaaacccca
tctctactaaaaatacaaaaattagccaggcacctgtaatcccagctactggggaggctgaggcaggaga
atcacttgaacgagggaggcaaaggttgcagtgagccgagatcacaccactgcattccagcctgggcaac
agagtgagactccgtctcaaaaaaaaaaaattatttgaccccatggccttctggtctgacgttttttggc
cgggcaccatggctcatgcctgtaatctcaacactttgggagctcgaggtgggtggattatgagatcagg
agttcaagacaaacctggccaatatagtgaaactccatctctactaaaaatacaaaaaattagtcaggca
tggtggcacatgcctgtaatcccagctactcgggaggctgaggcagagaactgcttgaacccgggaggtg
gaggttgtagtcagccgggatcgcgatactgcactccagcctgggcaacagagggagactctgtctcaaa
aaaaaaaaagttttatttctctaaatataataaacatggttattttatgttctatctgattattccaata
tctgaagtgtttgtgggtttgtatctatcatttgtgtgtttttgtggggggttttgccattcttctgaat
gtttctatcatctgttgtttctattgttcctcatagttgtttctctgtgtgtgttgtgattttttgacag
agttgcccattttcttcacatctttatccatgggattctctgcggcctgaattgaagttgagctcctcca
gagaagatcagagtttgcctctgctggtagactggggcagtatcaacctagactttaaatcctcacttgc
tcacttgacatttttcttttcttttcttttttttttttttttttgagatggactctcgctctgtcaccca
agctggagtgcagtggtgcgatctcagctcactgactgcaacctccaccccagattcaagcaattttcct
gtctcagcctcccaagtagcaggatttacaggtgtctgccaccatgcccggctaatttttgtacttttag
taaaggcggggtttcaccatgttggccaagctgttctcgagctcctgacctcaagtaatctgcctacctt
ggcctcccaaagtgctgggattacaggtgtaagccaccatgcccagacccacttgacatttttcagatca
tacaagtctcatgaaattaggccacaaacctgcagatcctggttgtgaattctcaggagaaaattgtttt
gccaagagtaatggagatcccattggtaataactgtaatattttcattgtgcatgtgtttgttttgatga
ataaacattggagtcatatatatctttcagccatga
Mouse PP1 subunit A (SEQ ID NO: 10):
aggagagggcccggagctggtgggccggagcggcggcgccgccatgtccgacagcgagaagctcaacctg
gactccatcatcgggcgcctgctggaaggtgggcaggggtagggaaggtgcgctcgcgccgagcccgggc
ccctcgccgtctggaagtggcgcgcgggcggcccgccgctcccgggggctagcgtcgcttgggcggggac
aggccgtgaggatccatggccacggcaccccccccccccgcgggcgcctgggctttggagcgcgctcaga
ggccgggggtgcacgagtgcccgggactcagggtgaggctcctgcgaagctagctgggaagggatcccga
agcgaggaagcttcggctgacaggctgctgagcacaggcgtttctctggaggccctccgggtggctaagg
ggtcggatcttgcctttgaaggcctgacgcaaaggtttcggggtgagggggtgggggctcacttgcaaca
gttgcccctgttaggggggcgcgacacctaccctcgtccggtctgcgaggtcagggtcccaagaccctat
tccgtactcagtgcagggctcacggcctgggaagaacgtgcagctgacagagaacgagatccgtggtctg
tgcctcaaatcccgggagattttcctgagccagcccattcttctggagcttgaggcgcccctcaagatct
gtggtaaatccttgcacagaacccttttgcttcctgaaaatagcccctactcgtgccccattaaccgtgt
tcctccaccctttaacctattgctggccctggggtcaagtttacatcttgcacctacctagttttctctg
gttcccaggtgacatccatggccagtactatgaccttctacggctgtttgagtatggtggcttccctcca
gagagcaactacctcttcttgggggattatgtagatcggggcaagcagtctttggagaccatctgcctgt
tgctggcctataagatcagatacccggagaatttctttctacttcgtgggaaccatgagtgtgccagcat
caaccgcatttatggcttctatgatgaatgtaagtgctttgaatgttgctgtctgcccagaactacattc
cctgctcttcatgggccccctacattggtctagtgccaaaagacccccaatgatgagccctgtagggccc
cagcaacacgtctgtcgtccttggcgctcaggaggggcctggtgaatggatgtacatgcagggctgccca
tttgctaaagccacagccctgcctgatgacacccccttctagtacattccttagtaaacatttaggatgc
ctgtatgaataaaacagacccactcctggtttggcctcatccatgggaccagagtgggtaccaccatcat
ggttgggccagacgagttcaggtgttggaattctggaaggtagatgagggtagagcaaagagaagaatgt
aagctttctttgcttttcggtctttttttctcagctttgtccctggatgggtgcacagaaacctagactg
gggttgggggttgggggtgtagtcttccctatgagccgcttccttttgcgcaggcaagagaagataccac
atcaaactgtggaagacgttcactgactgcttcaactgcctgcccattgcagccattgtggatgagaaga
tcttctgctgccacgggggtgagagtggttgtgggtctgtgcttgggttcttgggccacatccttagggg
ccttgtaaagttaacagaactcttctcagttccctggaatagcgctccttgaatctcatcttggatatcg
cttattaggggagattaccacacccccgtttgtttttgagactggctttctgggtaaaaccctgacagtc
ctggaactcactttgtagacctggttggccttgaactcactagagctcctgcctatctctgactcccaag
tgatgggattaaaggcatgctccacccaccagtttggttgcactctttcctgttgtggcatgaggcatcc
tgtagtcaactcattaatctccttctgcccccccacccccgaggtctgctatctgtacttctaagtggtg
gtgtcatataaagtgagttccaggacagccaaggccacagagaaatggggccagtggggaagtaatggat
tctgtctccctgaacttaaggagcccagattagcggtgggaacagtggagctgtcccaccccagagctgg
gcacaggacagcacttggtttaaggaacactattcccactccaccacccccaggcctgtctccagacttg
caatccatggagcagattaggcgtattatgcggcccacagacgtgcctgaccagggcctactgtgtgatc
tcctgtggtctgaccctgacaaggatgttcaaggctggggcgagaatgaccgtggtgtctcctttacctt
tggggctgaggtggtagccaagttcctgcacaagcatgatttggacctcatctgcagagcacatcaggtg
ggctggtggcctggggcgggctgggggtgggggtggggtgcctaacttacagactaaaccgttcctgccc
tggcccaggttgtagaagatggctatgagttctttgccaagagacagttggtgacactcttctcagctcc
caactactgtggagagtttgacaatgctggtgccatgatgagtgtggatgagaccctcatgtgttccttc
caggtgggtgtggggaagaggctgcccaatagacgcctggctcaaccccaggaccctgatgagtgttttc
tcttccagatcctcaagcccgctgataagaataagggcaagtatgggcagttcagcggcctgaaccccgg
aggccggcccatcactccaccccgcaattctgccaaagccaagaaatagcctccatgtgctgcccttctg
ccccagatcgtttgtacagaaatcatgctgccatgggtcacactggcctctcaggcccacccgtcacggg
gaacacacagcgttaagtgtctttcctttattttttaaagaatcaatagcagcatctaatctcccagggc
tccctcccaccagcacctgtggtggctgcaagtggaatcctggggccaaggctgcagctcagggcaatgg
cagaccagattgtgggtctccagccttgcatggctggcagccagatcctggggcaacccatctggtctct
tgaataaaggtcaaagctggattctc
Mouse PP1 subunit B (SEQ ID NO: 11):
ctgttcgttgcagtggaacgcctgagagagggaatcgctcctgcgcgttagcaaccgcggaggggagggc
ggggagctggagaggcaggaaggggacgtggcgcgcgtgcgcgcagttcggcgcggctaggcggcccgca
agggcggaggggagggagtgacgctgagggcggggctgtccgtggcgctgccgagctttgcggagctggg
cggtgccgaggaggaggtggaggaggcggtggcggctggggtctgacgcggcccggttcctgacgcggcc
gctgctcggcggggagggggtggggggagggcccgcgcccggggggagttggagccggggtcgaaacgcc
gcgtgactcgtaggtgagaacgccgagccgccgccgccgccgagaagccctgttaacgctttagggagga
gagtctggtgccgacaagatggcggacggggagctgaacgtggacagcctcatcacccgcctgctggagg
gtgagtgcggcgccggctcgtctccccacccggccgcggggcccttcccgctcgagctgaggtcggagag
gagggcaggcgggcgggcgggcgggcgggccgggtgccccaggaggccaggagcgcgggctggcgggcgg
gcaggcggggaggccgggccgagcgaggccggaggcccatcggctggtacagtcttcccgagaggcggcc
gggctccgcttggccgctgtgctggcctctgcagaccgttccgaagaaggggcgaggatgcctgccgctg
ggctttgtgtgtccgtggaatgtgtatttctcggttctctttgtgcgttgcccttggcagcctccgatgg
tcgttccggtagtgaattttacataaaaccttcccagtaaggctctcggatcgtttgcattttactggat
gctctagaaagaattagggagaggatacgatgactagtttaccatttattatctggagtataaaaaaaca
gtaaaaatcctgaatcaggttaaagacgtccgttagaaatacgaaacgcccggatacatgtgctgaaggg
gttgatttgggggcttttttttttttttaaacatgtttgagccgagtttaatttctgcacaaacttagca
aatgtctgttgtgaataatctcttgagcttaaaaggtattttacaagtgttctcagcatttgaagttccg
ggttcttatgcttttatcgattagttttgtgttggtgggaacataaggagaaaagaggagatggagaggc
cagtagttaaatgtattggtccgttgggggatactgttcgaagttaaaggcgggtcttggtcaaaagaac
ccttttacgctataaaatttgcgtcgtcactaattgtggagtgtgtaaaaaagaattgcgtttttgagtg
gttaagagtcctttttgtcagcctcctaatgaatgcgtgtgtttactacagcgaagctgtttgtgtttgg
aattttaccaaagagcgtggagaggggtgacttttctttaactagttaaagtacgaagtatttattctct
gtttaaaacaacaacaacaacaaaacaactctgtttatatgctccagatgtaaaagctttccagcagcct
taaggctgagaaaaaggcagaacttttaccaggaactctttcttatttaccttctcctttagtatcaagt
tattcctgggaggccccctggtgcttgctgtgtttactgcttgggattttgtggtctttaattgatagaa
acatcttgctgggtctgtgagaaatttgtttagttaaaaaaaatttatgcttgtagtacatggctggctc
tattagggaaatatagttattttaaatctttatatcttgttactcttcggagaaggggaatttaagccca
ccactgttttcatataatctaaattttaaattaaatttggatcagattttatcgcagtttattaatattt
ttaatttttcataaataccttatagcagatgcatgtattttaaaccgatctagaagcctttaaatcaaat
gtacttctatacagttacagatgcttaaatacaactaatagagtgggagttgtagagaaaaaggggaaag
tagcctgagctatatcattgttaaatatttggttctatcatcagtcaccagcacccaccagtcccccctt
cttgggtaagaatacgttctttttttctaatatagcaagtgtttattgactgcactgccaggcaccagga
gtcaaagataaggctcactcctcactcaagcagctcagtataaacaagtggcttctcagtgtgtgctagc
cttcctgctcttcctgctttgaatgtacaatttattagtagttttgttacattcttgttccttaatccag
acgaagactgcttttgttgttactctaaaatattaccagtccctccttccctccccaagcctaaacctgt
tcaaataaattttgaagtcttggattttctagtgagtaaattcaatcttgaaaaaaaaattttttttgag
acagggtttctctgtacctggctatcctggaacttgctctgtagaccaggctgcctcaaactctgcctcc
ccaaatactgggaattagagacatgcaccacctctgccgggcagcctactttagttttcagtattacatg
ctaatttaactcaaattcaagtggtgctattctgtaaagttgcctcgcagaacagaataacctggttata
tattttgtgcgtcctaaatgaaagtgaacattaaaagatacataattatagttaaagatgtctaggttat
atattttgtgtgtcctaaatgaaagtgaacattaaaagatatgtaattatagttaaagacgtctatccat
agcaagactacagttagtggtcattgtattgaaagtgtgaagagaaccttcgagaatgtgtgctctaagg
accagtatgataaatatgacatccttggtagataaaatgtgtggattcagtgtgttcagccatttcacag
tgtatgcacttaaaatatgtgttcctaaaagatgcagtgttgtctatcaattaaagattgcttttggctg
ggcaaaaagttgtggctttaatcccagcacccagtaggctgaggcaggcagatctctgagctctagacca
gcaaaacacaacaggaaaaaaaccttgcttttgtaaaaatgtttttttgcattaagagaaaaaaattgat
cagaacctagtctcattttaaagtgtttactttatgttacagtttattcatttaaaaatatattaaactc
tgatggccagttaagtgcactcactttcagtaactactttttatgttactttgttcgatgttagaccatt
taaataaaaagactagaacattgtttggactgggtgtgcttctccagtattattttcttggtttaagttt
tacttacaactaaacaataactgttttgccatctgtagtgaccctactatagtcttgtgatttatcataa
ttgattacataagttcagattagttctgtgagtgagctagaaagcctgcctagtaaaaggttttgcttag
agtggttgttctctagatatttgaagaggaagtgggagtcacctgggggccttaggtaacattccaaatg
agtttattttgaaagaagtcaaaagtaagtagaatcctgatactttgtgaaagagaatcaaatggtgccc
ttggaggccagaagatgccctagaactggaattacgatgtgatgagccgccctgtgggtgctgagatttg
aacccaggtcttctgcaagaacagccaggctcttaacggctgagccattgctccagcccaaaccttccct
tttaaagacaactttgagatgcagttcatatcccacatggtttgcctattgctgctgtgttagtcagtga
gttttagtgtgtccacagtgttttgcagctatcaccactgtcagctttagaacattctccctcccttcca
cctccccccacccccacaagccctgtttcttttttcttcatctgtctccctctaggcagtagagcggatc
ggatcgttaatctgccttctttctccagatttgccggtttatgcacatattatgtaaatgaaagtgtata
taatgtggtcttttgtgactaattttcttttgagttttttgagttcaagtttttgagacaaggtgttact
gtgttgcctttgctgtccccaaactcagagatcctcctgggattaaaggctctgtaactcctgggcctct
tgtaatgttttcaagtttcaactatgttataaaatgtattagtacttcattccttttatggctgaataat
actccattgtgtctgtgtaacaattttcagaaatactcactggttgatggctatttgagttgttttccct
tttgacctttgaggacgatcctgctctcaacttgtttttgagtggatttaggctttcagttccctaggct
agattgctgggaggggaattgttacatggtaactcggtgtttagacttttggaaaactggagagagttaa
agcttctttattgattacattgcttcaagttcaagcccagtagtttaaaagacaggttgaagcaggtgcc
agctgctctggctgcctcttccttcacagggaacatcttcatactgtgtgtgaacatgaggtaactcatt
agaagtaagagcaggctggagagtagatcattggaggtagactttcttctcttgagtgttctgggtgagt
catagatagaccaatccctgtttgtccagaaagtaaaggaaactgatttgatttgagaaaaaaaccaaaa
aaccaaaccaaaacaagaaaaacctccagagatattttctgtgacatttccaaataattcattgaaattt
agagacctattcgaattactatctgcggagagaagagttactcccacccctcctttaattactaaaggac
attatgttgaagatactctaccggtgagatacttctccagctcttttatttttatttatttatttattta
tttatttatttaatttttaacaagttttgctaagagagtagccattctggccctgagcttgccctgtatc
ccaagcagaccttgaactttgaattttctgacctggactcccaagtagctaggattacaggtgtatgccc
tcatactctttacatttatcatttgactattccattttcagtggccatggtggcaaattcataccagtaa
atagaaacgttaaggtatttttatatgatgaaactcccattgagtatttctgaaacaaaagttttagaat
taattcagtgtccgtaggcaagaaatggtttactgaagtatgtttggtagactcttgactatcctgggtt
ttttagctttgttagggtgcaaatttctaacctttggcaaccacttttttttgtaactataaataaccat
ttcagggcttgtttcttttagaaaaaaaataggatccatgaaggttggaaagagaggtcaaggtcactaa
tctttctgtgtatcacttcattctgaaactgagttgccatgtttccaaagttgtttttagtagatgaagt
ctaatttttacagctgttttctgaagtacattccaaaatagaatctccatttgattaatgagatgggaaa
actgagatagtctttattctattatgggaagtaaaagacccaaatggagaaacctattctgaagggaaag
aagttataacagcactagctaggtttggtgggggatgatctttaattcttggtaggaatttgtaatgcac
gggtgttgtcctttcttctagcttttcatgtgtgtagcacagtgatttataaagtagtcctaacttccct
gagtcactggccagagagattttcaaatttccttgccctgtataataaggaaggacaagaagacgtgtgc
acagcgggaacatgcttttggtggttggtccgtgttaagattgtaacagggaagttttgacccagcttat
ttcaccataaaggagttttattccaagagcattaaaatatcccagtgtaacagaagctaccaccagaata
cctacaaaacataagaacttttatgattaatttatttgatacaatagtcattgcaatttagtatgtgatt
atttgtgaaaaaaataacttcataaattattgaggatgttgttattcatttgtgtaaacctaaagtctgc
acaagagacagctaaattcttacctctctttttttcattgttgttggttattgtattttgtttgcttgtt
ttgagacggggtctaatgtagcctttgatggtttggaattttctgtgtagataaagctggtctggaactt
gcagggatttgcctgcctctgcctttgcctcctgggtgtcaggttcagtctgttttgacccatggccttg
gctgaaatatatgaagaaaatctgtatttgtattgtatctatgttggtattttaaaattacatttatttt
gtatgcatgcatgcatgtttgtaaaagtcagagggcagcatttggaagctggttctttgcttctaccgtg
ttggatctggggtgccaactcagctaatcaggcttggaaacaagcagctttgcctgtatctgtcttttta
ctgaattagaaaatggtgtctttgccagccatggtgcacatatgtaatgtccaacactggaaaggcagag
gcaggcacatctctgtgagttaaagttcaccctgatctccacagtgaattccaggtcagccagggctcat
agtgagataccatctcaagtaagtaagtaagtaaatagccaactgttttagacaattgtgagtactttct
cgtactatcaagaatcaacaaggggctttttcaattgcaatacaaattataatttttttctacattaacc
taaatctatcttgtactgtaaatgttctacacatgacttggtgatgggcattggtcgtttggaaaatggc
tcactgaattatatagatttaaactcatataaatgtcagtagagctcatcagaagagacttttttccatt
tttattaggtatttagctcatttacatttccaatgctataccaaaagtcccccatacccacccaccccca
ctcccctactcacccactccccctatttggccctggcgttcccctgtactggggcatataaagtttgcgt
gtccaatggtcctctctttccagtgatggccgactaggccatcttttgatacatatgcagctagagtcaa
gagctccggggtactggttagttcataatgttgttccacctatagggttgcagatccctttagctccttg
ggtactttctctagctcctccattgggagccctgtgatccatccattagctgactgtgagcatccacttc
tgtgtttgctaggccccggcgtagtctcacaagagacagctacatctgggtcctttcgataaaatcttgc
tagtatatgcaatggtgtcagcgtttggatgctgattatggggtggatccctggatatggcagtctctac
atggtccatcctttcatctcagctccaaactttgtctttgtcactccttccatgggtgttttgttcccaa
ttctaaggaggggcatagtgtccacactttagtcttcattcttcttgagtttcatgtgttggacatagta
ctaccggaggatccagcaatacctctcctgggcatatatccagaagatgccccaactggtaagaaggaca
catgctccactatgttcatagcagccttatttataatagccagaagctggaaagaacccagatgcccctc
aacagaggaatggatacagaaaatgtggaacatctacacaatggagtactactcagctattaaaaagaat
gaatttatgaaattcctaggcaaatggatggacctggagggcatcatcctgagtgaggtaacacattcac
aaaggaactcacacaatatgtactcactgataagtggatattagccccaaacctaggatacccaagatat
aagatacaatttgctaaacacagaagagactttaagaatgggaggaggtattagaggagacaaggtttcc
tgaaatcttgaacttcatcattgcctacaagtactgttttctttgaaatgattttgtttgatttgagact
atttgttaattatgtaagtctggtaacaacagttttcagtcatgtattttcctttgaggcaattactgtg
ttgtatgcagtaggtatacgtctggatactttgcattttcttgtatagacaaagatataatttcaggagc
taggtgtaattgatcatttttactgtttttatctcagtattttacagaattgcatggcagtgaacacact
agattcctagtagtttgatacctctggtcaggtgtgtgtatcagagtgtgtagagtgttccttcaatgtc
agcacaagaggagttcttactttgaactggatacttctttattcctgagcagcagcatcccagcctgttc
atcaacatcttccattcttcatgctacaccacctgttttgtttgccagtgtgtgggtgggggaggggagt
attacttagagtgtatcacagatccctttgagaattcatgaaaaacactaaaagtgttacttttgctctt
gttgctaatagacttttgtactcgatatagaagaattgcctatacagaatttaggtactctagagtagct
gacattagtcagtgatggggtgatgaggggaggtgggtcgtaaatgagatggaaatatctaagatttcgt
gaggcacacctgcaaaggtatagaattggttccttttattttcctgttgtttttttctgttagagtaaat
tggagaaagtacaggtgacagtgaaagataaactctagtgtgcatacaggatgcatgaagatacatcaca
catctttcttagttcaaaagactgtagtagaaaattttcacataaaaattgtactaaagtggttaagtac
agtttgggtttatgtaattactggaatggaattcttttggagagactaacattttacttaagattcaaag
ttagccttttcagtattgatcatcacatttgacttaagtgctcatctgctagtggtgatctgtaatgaga
ttttatgtatttaatcttggaagatgtcctttcacagttagagccaccaaatattaacagtagtctataa
gcatatgattttaattgagcatattcattacttagaacacatttatacaattatttagacatttgtcttt
taacatgctgtgttacatatgattttcacagctgggtgaaagctgcttagctatacggtaaacccaattt
taatgtggaaattgcttttgcacctaaggtctgaaggaggctgtaccttgaattaagaaacaatttgctg
tagatgtatgagaataaagatctaacttattttgacttttgatagcatgggaaatgaaaatgctattgtt
aaaatgacatgcactatgtttgcaaactgtggtgggggagttgttaaagtttgaagttgaacttgttaag
tagcattaagttctgggtagtgaggtcacacatatgtacctgtaactcagtagtctgaggtaggaagatc
ttgaagtcagtgtagattacatatccagaccctacctaaaactattctgtagtggtaatgattaagaata
attttcatgggttgaaaagattagcagttatgaacacgtgctgttcttacacaagatctatatttggttc
ccagcacccaacttccagtaactccgatgctaagggatgccacacctctggtctccgtagtacttgcact
catatgctcataacctacacacagatacatttattcgcataaaataattttcattccgaaacctttatta
agttaaaattactgccataatcacaaaactagaaatggttaagtttatgaggacaaaagaaattggttac
tgatactactggtttgataacatgataggttaaggtatttttctgcagagtaactatcataggtttttaa
agagcctttacattttcacaaactttatagtttgtccaactaaggcctttccccctccccattttatttt
taccaccattgggtctgctattagttatactgagttgttttttctcagcttctttgaaagcattcttgct
gtagttccacactggctatcctagaggctaactttgcagtctcagtttgaacactagctcctcaaacagt
ttagagtgttagtaacaactgccaactaaccaggactatgaaaaaacactcaaagccagctttttaattg
ttgatattgtcctcagtttccttaattggctgatagtctttaaaattgtctcaagatatttttttaaaga
gcatcccaatcaaacattgacctaacattggtaaaagagcttacctcagttggctgacacttgaaaattc
ctttttgaattccaagtttgaggccagccagaactacgtggtgtgaccctgactcaaaaacaaaactaag
caaccaaacacatgaagaatgctaccagagctaccagtcatgccctttaggtaacaacagaaaggataat
ggcccatctgtcaccgaaactacgaaatcccaaaccgaatggcttttaaacagcaaaagaaaaagaaata
cttagctagaattccaaatctagtaagaccatccttcaccggtaaaagcaagttagaactgagaaaattg
gttggttaaagggtctggaaagatggctcagtggctaagagcagggctgtagtgtgagtcccagcactca
catggcagcttgcaaacatccgagatctgagcttcgttggcatctgtaggaggcatttcaccaatgtgat
acacatacatacatgcaggcaaaacactcactcgtacacatacaattttaaaatagagggaaaaggtaag
gaggagcctggcatggtggcacaggcctatttttaaaatgtattgtgaggagagggacaggggatggatg
agttcttgcaaagcaccagaaaagtggaatccaaaagatcaaaggccagcctgggttacatgagaccttg
tttcaaaagcaaacagtaaatgaaagacaaccagtggaggtgcaaataaaatgagttggtgtccccaccc
caagaaatgcaaaaaagaaagaaaaaggaaataaagagcagataggttagactggaaacaaatagtagag
cgagaaagtctgacctgacttgtctttacaagtgaatgtaaattaaaaagaatctaatcaataccccaaa
atcttgagcaaatttgtgtcaccatgtaacccattaatgtgtgttaataattacgatcttaaaaacattt
gcatgttgaatctggctatgtataaaagatagcatgtcaaaaactaaccggcttgttttaggagtccaaa
ttcactaatgctgagaatgtcagttaaggtaaatcatgttaactatatatatatataaagacttagtaaa
ttagaaacaagcttatcttcaagataaggaacataaagatatagctgtcagacctgacatgagttgctga
tgctatttcctggattcaggaacaacattacttctgttcctggaagttctggccaattcaacaaggaaga
gaaaataaacaaggattatatcttagtcactgttctgtggctgtgaagagactttatgaccaaggcaact
tttggtttttcgagacatggtttctctgtatagccctggctgtcctggagctcactttgtagaccaggct
ggcctcgaactcagaaatccgcctgcctctgcctcccgagtgttgggattaaaggcgtgcgccaccacgc
ccggccctgcaacttttttttttttttttaattattttatatgagtacactgtagctgtcttcagacaca
cctgaagagggtatcagatctcattcattacagatggttgtgagccaccatgtggttgctgggaattgaa
ctcaggacctctggaagaaggtcaaaagcaatcaaaagcaaaaatcaatctccaaccatccaagtgtctg
ggatccaactcatgatcttctgggctcctccaagagcttgggtcatttctccagccatgccctttgtagc
acccgtgtcgtcctctaggctccagatgcctgtactccactgctgctgctcttggtggtcatctcatggt
actggcatctccaaaacactgcatgaccccttcagtcctgggcggtcaattgcaactgaggctgcacttt
caccaatggccttccatggcctctcacagtgccaagcctcagctgctctgctcaactccttcatgccttc
aaaaccagtaccacctgggtgacccttacacattaccaagtccagccacagcacaaggtacaactttggc
tatatctggaacaaagccactgtgctctcagaaaacacttcccagaagatgtcacctcaatgatgctggt
ctcttcttaatcaccgctaatttcttagctccagctaaccagcatcaatagtcccagtaatgcaaagttt
ttgcttttgtagttctggtatcttgttaatcacagctgattcttcagccccagctaaccagaactacaga
atcttcacaatcaaaacagcaatggccttgaaaagagtctttaattttccctttgaaatttcacaaacca
gacctccatcttctgcagtgttctcaacattatcttccaagctcctacacaatatccgacagagctctta
acaaaggatggatcttcaagcccaaagttccaaagtcctccctcagtcctccccaaaacatggccaggtt
gtcacaggaataccccactatgctgataccaatttgtcttagtcagggtttctattcctgcacaaacatc
atgaccaagaagcaagttggggaggaaagggtttatttggcttacacttccatactgctgttcatcacca
aaggaagtcgggactggaactcaagcaggtcagggagcaggagctgatgcagaggccatggagggatgtt
ctttactggcttgcctcccctggcttgctcatcctgctctcttatagaacccaagactgccagcccagag
atggtcccacccacaaggggcctttcccccttgatcactaattgagaaaatgccttacagttggatctca
tggaggcgtttcctcaactgaagctcctttctctgtgataactccagctgtgtcaagttgacacaagttg
ccagtacagcaagaaagcataatgataaaatataggaccaaagcaaaactgacaccaagcaggatagact
ccaagttctgtatctgtgtctgatgtcaaaagtgttcttcagatgtccagttcctttcagttttgttgac
tgcaacacacctctctcctggactagttccacaatctgataacaactctccttggcaggtatcccatgac
tctgacctccagcatcttgggttcttcaacactgtccaggcttcactcacaccttcatgcagtctatact
tgaagtctttgaagtctcctctgcaatgacattattccaaaactctttaattagcctcaggcagatgctt
aggacaggggcaaaaagttgctacattctttgccaagttatcacaagaatgttctctgggtcagctgata
aaactcttgtgctgtgctgacatcattcaaattagcatacttgggtgtgtggtatagttaaccattacta
tctttcaggctcctactagaatgttaagccccacctttcaactgctttcctaatccatatcctaaaagtc
tgcttctaccaacaagcggccggtcaagcttgtcacagcaataccactcctggtaacaacttcttagtta
ttctccccttgctgtgtgagaccccagtgaccaagacaactttttttttctccctctagacagggtttct
ctttgtagcctggctgtcctgggactctctgtaaaccgtgccggcctggaactcactattcacctgcctg
tggttcccaagtgctgggattaaaggcatgcactatcatgcctgactgcaaggcaacttttataaaagaa
agcatttaattgtgggcttgtttacagtttgagaggtttactccaaggtcattatggtaggaagcagata
gcatggtgctagagcagcagctgcgtggtggtggggtgggcacagtggactgagaactgagcttgggctt
ttggaacttgaaagcacagccctagcgacttgccttctccaacaaggacacacctccaatcctttcctaa
tagttcatcagctggggactaagcatgcaaggatttgagccctatagggcacattctcattcaaaccacc
atggattagaaaaggattcaaacttttatagcaaatgattggccttaaaattaaaaaaaaaaactgtaaa
cttatttaataatatacaaagtcagtatacaaaattaattcctatatatattacaattacataatatcac
atagtgggaaactaaaacatttatactagtcttctcaaaatcagcttgatgaaaaatactcaagactgcc
atagtaaatgcttcaagaggaaaataaaccttgttgaggaaaacaaccaaagcttgtaacatagcccagc
aagtaaaggcacacacaggcatgcacacaagagaaattaaataaaagaataaatggctaataaaaggcaa
gggaaggtcatgggttagaagaagacttaaaattaagacatcagttctactagactcagttcatttcaca
aatatcccacaagccctacagatttgggggtcaaacaaacacaggccaattttaaaactttgggaaacat
aaagatctagattaccctaagaatgttaaagatcagaattgaagtgttcttaatccatgtattgaggctt
tacaaaaaactatgatggtgtgatgatgtaaagagactcaggcactaagaagggtggtggttgttttata
agctgccgatgctgtctaaggagggaaaggatcacctacaacacgtgctataactctgggctgaagagat
ggctcagcaattaagggtcttccagaggccctacgtacccatataatagctcacaactgtctgtaactct
aatttttttggatctaacatctgtgtctttcaccagcactgcatacatgggtatatagacatatatgcag
gcaaaacacctatacacataaaataatcttaaaaaacaaattgacttttacttcaagcaatataacaacc
taacttatgatatacaattaattaaccaaatgtcaaaaaaaaaaaaaaaagaaaaagaaatgctcttagc
aagggtagtggtttcttggtaagatgtaaaaagctctaagaagcgaagacaagattaacctagttgaaat
tgttttaaaatttatgttcattaaaaaacttctttttgttgaaacagtattaagagaaggggaaaggtcc
atcgcgctttaaagaataaataagcaatatttaaatggcccactcattataggaaaaggcactcaaaacc
ttagtgatcagggaaatgcatgtcagtaaatgttgctacatagccaggagagccattaaaatgtaaaata
gggataatagctagtgttggcaaagttacagagcagcatgatggtttattataaattgatacaatcacta
cgtaaactgctggtgtctcttaagtaaaagccttctctgtgactttgttctactcagtgttaaggcatag
gagaaatgtacttgaagttcttgataacattgatggtagcaccaaagtgacccgtccttgagtggagtac
tgcaggtgaccgtacagttacctgggtagtaggtgtcacagaaaagattatgagcaaaagagtttatttc
tgttttctaaaattcaaaaacaggtcagattattctgtagtgaaggaatcactacacttggacattgtta
tttagggcaaaaaggaataattgtgactgcagggaacagcatatcctgagctgagaagtagcatacttgg
gtgtgtggtatagttaaccattcatagagttatatatttgggcgtagttatgtatacagtaccttagaat
gtcttttgtttagctatagtatagtagatgtttgctatattttgattgtacttagtggctttattgcaga
ctgaaggattttaccatgaaattttatctataaagtgaagaaagtactggttgtgacatgttctgaattg
ttcctgttgttttttgctttctaagtgttgtgaaatccaagtgttgtgatcctaagttatcaaatctctt
attgtatagtgagataacttgggctttctgggataccaaaagggccaaatgacaggagatgatagtgttc
tgagcctaatgaggtttgcagccttgactgtatctttataatgtgatgtgaccttacatctgaaccttta
attcaaacactggagtcccgtgaatattgtgggctgtgacaggaacagatgatttctttatggtacctcc
acccctccaaaggtgactgagaaatgtgacccaagagtactctttgacctgagaggtcttcagggtatca
ttctgaactggaagaattgagctgctcaaggactgccttggttctctaacatttggccaattcactttga
gatttgatcacttgctaaaggcctttgaagcctttagtttctgatagcatttcatcagtccctaagatct
acttcagtcgaaaatgtacctttatgtgctactaggtggggagaaaaattaatcctaagaagctgttggt
tagaaatacaacttcagctggcgtggtgcgcgcctttaatcccagcatttgggaggcagaggcaggcaga
tttctgagttcgaggccagcctggtctacagagtgagtcccaggacagcctgggacacagagaaaccctg
tctcgaaaaaccaaaaagaaagcaagaaagaaatacaacttcagccaccatcattgcaacatgattttat
ttgatttttagttttgtagattaatgcattttaaactttgtaaatatgttaactgtttagaattacttgg
gggttcaaaattaaacctatagctggggaaatgggtctgcagataaaagttctttgcccaacatggagac
caccatgtggttgctgggatttgaattcaggaccttcagaagagcagtcactgaaccatctctccagctc
cagagtccttttcacctcaagcacatgccattgtgccaactttaaatgtttttgtggtaaccaaaagtgc
ttttgttggcttcattgctactatttttaacaactaatacacagtgggcttccaagtacttagtgaattg
atgacttctgaacaatcatttttagttctgcatttacttgaccagaaacttttatctttctcagtgcata
cttttcccacttaaactaaaagtttaccaaattaaaactaatgtcaaacctagtatggtgccacacacct
tgggagacagagacaagtagatctctgagctcaaagtcagccagagctacacagtgaagcagtaaaaaat
acatacatacatacatgcatgcatgcatacaggcaaacaaaatccaaatgccaaaaccaggtgtagtggt
agatgcctgtaagagctagggctcttgggagatagaagttagaggatccagagttcaaggccatttcttg
ctacacagtgagttcagactcagcccagtctatatgagatcttgtcttaagcaaacaagggagtgtgtgt
gtgtgtgtgtgtgtgtgtgtgtgtgtgttggtccttccttccttgcttgaacacattgaatcaagggcat
gtgtatgcaagtgtgaggacagggacagcctcttggtgtcattccttagttgcctccactttaattttgt
cttttgagacagtctctcactggcctaagaattcacccttgaattcaaagtaacaaacaatcagcaaaac
tacttttttaaaaatacaaacccaacacagctttaaatgaatgctcagattacagttgtgaaaaacttgg
gtttttctaagcagtttaggctggctggccagggagagccccagggctctacctcctgtttctttcttcc
tggtgctgggatttcttcaccctgctttttaacatgggtttttatgcttttaagatagtattattagctg
gcatatttgccaaggtacatattaaatgacccacattcatcttttgatttaattataattgccattaagt
ttcaacttatgaacaatgttgtattcttttatctttcttgctagtttgatgttaaatgtagtcacagaat
ataatacaattttaatacgggtgcctgggttgacttttggatttttgatacaggctttttctgaagttca
ttctgtagaccaggctggccgaaaaatcatggaatagagatccagctgcctctgtctcttgagtggtggg
atcaagggtgtgtgccaccactgcccagcctgactttttaagcctgtttcagtctgacaaatcatagcag
ttactggggggaggggaggaagggattgaggtgggtaaagaagggggggaatcatttataagtggcaaat
agcaggggtggcatgttgtagtgtttattttaatcaacaactagacatgtggtcggtaggcttaggtgct
gctgatacttgatggagctgtttgaagctggatcatctcactctgttcatggctgctggctccctagtga
tgctgttcttcctgctcagtgcaaggcttgatctaatgggagtaagctaggccttcagagatgttagcct
tgaagtctattaagtatcttggtctactagctcatgctgtgtccttggaaaccagcaacattgggaatta
atatcacagttcataaaataaactcattgttagccatacaacccagtccatgtgtgatttttttttcctt
ttttttttttttttgtcttcaaaatatggatgtagctactatctttaagcatctttattttatggataat
tttgcaaagctgatgagcttcctcattgtttttaccagtacgaggatgtcgtccaggaaagattgtacaa
atgactgaagcagaagtccgagggttgtgtatcaagtctcgtgaaatctttctcagccagcctattcttt
tggaattggaagcaccactgaagatttgtggtatgtaaatggggaaagttaaaaatgagttttccaaagc
acattttctcctgtatatatagtaagaatgacttagtgaaaacttctaaatttgataggtttattaaatt
ggaatccattggaattcataataatgataggttagaagcaacagaattgctatttgtagtatataaaatg
gccctggaaataaaaagaatagtgttaagaaaaatgggcagattctaggagattgctaaaatcagtggac
tgcagcttaaaacacatccctagctcatcaaattacaaacaaaaagtgccagagattgatatttagtgct
tttaaggatgtgatgaaattggcattctagaatgctgcttagaaacagttaatagggccaggcagtggtg
gcgtacgcctttaatcccagtactccagaggtgcaggcaggcagatctctgagttcaaggccagcctgga
ctacagatctgagttctagggtagcaaggactgtacagaaaaacctgcctggaaaaacaagaggaaagaa
gaaagagtcatggaatcttgagtgtcagcgtggcaggatccatggtcttctagcttgtagtgattttctt
ttcttttcttctctcttcttttcttttcttttttcaagatttatttatttattatatgtaagtacactgt
agctgtcttcagacgcaccagaagaggcaccagatcttgttatggatggttgtgagccaccatatggttg
ctgggatttgaactcaggaccttcagaagagcagttggtgctcttacccgctgagccatctctctagccc
aaatgattttcaagatactacttagacgttgtgtttgttctcttaagtcttatagttgttttagcccaag
acaaacacttagagagaaattattacattaaaagtagcatttttctgtagttcacgtacaacaggaagtg
gtataaatactacagcatgctggcgtttcctttggtgatacaggcagtctttgtaatggttaaagtgtgt
ctgaagaaacttctgtgcacttagaatagcttttgccatatgcaaaaatgtcctttttgcttgtttctaa
gcttgtttctaccttgaatccatataagatgtttggggcattttcttctaactgctcagaaatgtttttt
gctaatggggtactacttactgtcctttcccaggctgctgccctttccataccagggttccactgagctc
agtatcataatgtcggggcaaaggcactgggaccctggcacctggtgtggaggagaaatttgtccctctc
tggggattgttttctggtctgttgttaggagcaaagtgggaagggacacattccatcaacactcaaagct
ccataatgtccctcagctggggttgcagggtagtggaggctaggagtatctcctgacctcagcacatcag
gcttctaggatagtgtcagagaagcagctctgggcaaagtgacagggtttggggaaaagtcacaggggac
tgcagtaagtttggtcattatgtaggtcgagtcaggagaaataggtgaccatgttcttcttggacaagga
cgacttgtggaagggctccctagcgtccaggctgagcgttatcccagtggttgcctacttgttccaactt
ggagggcatcctcattccagtggctattgtagaatcattgtggttagattgagggtactgcagatttaac
atacatctattgttctttgtattctctttctctgtggggtttattaagtcatgtgtggtgccttgtacag
tatccgtcaaataaataggtgcaaagtagatgttggttagtgtgcttagaacatcacccttaaagcaaaa
gcagtagtttgaaactttgtagaaatgttcagaatcttgtgattattataaataaaataataacattaag
ttgaaaatataataagccccaaaatatatttagaacactaatgctgaacattatcacttgacacatcaca
ttgtaaaccatcggtattttacatctatgatcacatgactggtgggagttagagcttgctgctgccactc
agcatctcccaagtgttgtactttcactagcatgggaaagtcagcggagctacagtgcttataaagtgag
agtcttgtaaatgaaaccattttaagtcattgtttgtatttcttactagtaccccttcggtgttacttaa
tacagcaataatttgaggtaatagaagtattttagctcttgatatttggtataacaagtatgacttaagt
ttagctatcggctaataagagtaactgtgccagttgtctattattttattagaaattgcctgacattttt
gtggggcacaacaatattagcttatcatcccagtttttataaggtagggattcagacacagctttgctag
atcttcaggttctaagtcctagaagcataaaattgcaatcagttgttctaacctgacttcttttggtttt
aggagacattcatggacaatatacagacttactaagattatttgaatatggaggttttccaccagaagcc
aactatcttttcttaggagattatgtggacagaggaaagcagtctttggaaaccatctgtttgctattgg
cttacaaaatcaaatacccggagaacttctttcttctaagaggaaaccatgagtgtgctagcatcaatcg
catttatggattctacgatgagtgtaagtgggatttcaaacacaggagaggaatttccatatgcttgtac
agttcagatgaatttcttcaacttagagaaactaggctttttgttttttaaaataggatctcagacaaag
acccagaaagaagagcctaaagctaggaaacacattaaattgttttcttgggatctccttggtcatgttt
tcaggctggctacagcttagttgactgaaggaggcctcttttaattagtttatctggtaaaatctccaag
tgaaacaaatacaggtcagtagtctccatttttctgctgtttgatagggttcgctttttacatttattca
ttattaatatgttttgtgtgtgtgtgtatgtgcatgcacacacatacagtgcatacatggtatcatgaca
cagatttggaggtcagaggacagcttatagttagtcctttccttgtaactagaagttggaagtagagaaa
tggctcagcagttaggagcccttgctggtcttcagaggccttcggtttggttcccagcacccacatcagg
agactcacaactgcttttagctccaagttcagggaatcccattctctttatggtctgggcaaccacttca
tgcacatggttaatacacagaggcaagtaggtacacatgcatacacataaatagaatgctaaataaaagt
caaactttattattaggattttaatctgtttatatacctagagtggttactgagtccttggttatatagt
gttcttgtttgttaatcagtctgatttgctgctgtgtagcagtctttccagtgtcaactctgcatttgga
tttgggttttccgtttaattaaattaaatatgggaataggtgtatttagtcacctatagttaagtcattt
ctagtagttttcaaatcaaatttatttttgactgctaaattatctattagaatctattttttcttaaata
tggtagtcttcaacaaactcatagcttaattcctgtgatgatataagaaatgtttgtaaatgtgttgtat
atgctaatcacttctaacattggaatgtggtaaaattcaaggcaagttactttaaaaaaacagaatttat
gttatactaggaatcattctgtatttgagttttacattacatttttgtgactttaaagtcaggcttgtgc
agaggaagtaagttgaagtacatcaggtgatagccatctttctatgaggtatattagtatgttcttagaa
gataacttaaggcctgaatgtccctaagtgtagaaaggacatcagatggaacgatgtgtgggaaaactgc
ttgtaaacagtctataaattgtacttaatcattaaaaggtagaataggacataaaacatgaaattaatag
ttgtgactttcatcctttctccggcttggtagttgaacttaatgccaaatcaggtaactctcagtggaag
ctgcctttgttagagctgggctatgggaaaattatagccttgtgtcttttcacttgccgtagtgacaagg
tgtaatttgttctaaagtgaataccacttaggcacttggaagtgttcctttgctggcagtaggtttatac
ttcattactcagtgcattaagttaacgtggactagctccagaggtaccattttcacttatgtctacttac
ttagctaggaaatgtattgaatgggaagaaattagctaatattctattagatgatttcagaagtattata
ctatagtgatttttataagataaagttgtttgatgaatgtgtttcatagaacagtacagtctttgaagtt
tctgcaactctacagaaggtattgaatgttttattccccttttagaactatctaagaaaaaaaaaataca
tgtggtgataaacctgaatgctggacaagtactcagatatgagctacatctccagtgcatgaaactgaac
atagcttattcacttacagaaaaaggggctaagtaattgtcttagtcagggtctctattcctgcacaaac
atcatgaccaagaagcaagttggggaggaaagggtttattcggcttacacttccatactgctgctcatca
ccaaaggaagtcagatctggaactcaagcagggcaggaagcaggagctgatgcagaggccatggagggat
gttctttactggcttgcctcccctggtttgctcagcctgctctcttatagaacccaagactgccagccca
gagatggtcccacccacaaggggcctttcccccttgatcactaattgagaaaatgccttacagttggatc
tcatggaggcatttcctcaactgaagctcctttctctgtgataactccagctgtgtcaagttgacacaaa
attagccagtacagtaatcaagcacagagggaaaaaaaacagttattgattctttaattctctgtgtttt
ccctttttataggtaaacgaagatttaatattaaattgtggaagacattcactgattgttttaactgtct
gcctatagctgctattgttgatgagaaaatcttctgttgtcatggaggtagagtaatacatttgctgttt
agaagagaactgtaataatgacagtactgtgggaaagctgagtgtggcaggaggtgtagagcaactagaa
gccagcttgattccaggccaggcaggcttaggaacaccccgtctcaaaataggaaattcttcactgggcc
ttacagagaatgcctcctatacaagtgttaacagaagagtggtccacacacactggaatcccaggactca
gaggctgaggcagaaggatctcaaggccatcctgggccatagattctacctcagaaagaaaagagaaggg
aaagagaaaaggtaattagatgtgtgtggagtatttttactttgcctcaatttatgcttgaccagctggt
cagatgatttcctgtggtgctgggatctagctcatacctcagattttaataaaaggtacttggccatcat
taagaatgagaggctaattatttttctgtctgtattaactaccttttatcaaactctaaacataattcat
acagctttccaagttttgtgcactacgccaacatgttaggaaatgaggacattagaatatagagattaga
atttagatcaaaaacagttttcagctctccagctttagcacccagctttaccttggtgtgtttctcattt
atgctctgaggatcataaattgccatgactaatgactttctaaagagcttcaaatgtgtagtagtcatga
acaatttagtcttctaaagatgaaagttttgtaagtttacacacatgagaactccctgaagaatatgcag
taaacttttctatgatagtacgtgtacacacacacacacacacacacacacacacaaaaagtttttcaaa
agttagggaagattatggtaagggcttgctttgaaagtcctgtgacctaaattcaatccttaatcaggga
tcttgaggccaatgaggtttcttccttagaacaaggtagaagcaagtacacatttactctcagcacccag
gaggcaggtaggtatagttctgagttcaaggccatcctggcctatgtaccaaggatcatgccagccaggg
ctttatagtgagaatcagtctcaccacacacacgcatacatgagcacacatttacacacactatggaaca
atttaatgatatacaggaaaaatggtttcttggtatcatacatggtagtgtcttttgggaagcatagagg
tggaaatgctgaaagatggtggcgaggcctgagacgtgagtggtttattctgacagcacagttcactcta
ttagttgaaatgttgttttccatagatactgaccattaaaggcattggcagagttgaggtgggaatatgg
cagtgtggaatatgtgtatttagcttcatttcttgtggtacttgaattacctaccagagaaataatacct
tatgttctactttacagatatattcttgtgcttttttttatccttctgattattagtttaatgcacattt
ttgcagataatgagactgttgaggtgggatatggggaaaggttcctaggggaccaagagaggcgtataac
ttgcaagtaaaaatgctttatggggatctggtggtcatgaaggtacagaagcagtaggaaggttgcaact
aagtgtgttctcaggaagggggagggcaactgcaaaaaagtagtagagtttctccttggaatctctcgaa
gaataatgatttatttccttggtagaaagatagaaaagatagcatgagtataattctgctagatagcaag
tctctttctttctatatataggactgtcaccagacctacaatctatggaacagattcggagaattatgag
acccactgacgtacctgatacaggttagtatatacagaagttcagttcattgctttacataagatgttga
cgtgcttggacctaattatatttagtgaaagtcacttagaaacctagtttcatccctgaaaacagttaac
aaagtgatcagagcaagaaaaggagccgccctgccagtcttaattaaagtcattaatgtgttcatgtctt
cgtgataaatcagtaaagtattgcaatataggctggtgctcagtagtagtgtaatgcaaggagtggataa
gctgtgtacaggaaatgctagccagaaaggaaataaaggcctccacgtattacatatacttgagtatgcc
acacacatatttcctgtatgcagaagtcaacagaaagtactaccctaggaagctagagaaagttgtactt
taacaaagactggtttagactggagtgcctagggacaagggatgatcattgaagtttaagaaggcaggag
agatgtgctgtaaatgtgtgctgtattctgtgcttgcttctatttcactgttggtttgtttgtttttttt
ctgttttctaaaggtttgctttgtgacttactgtggtccgacccagataaggatgtgcaaggctggggag
aaaatgaccgtggtgtttcttttacttttggagctgatgtagtcagtaaatttctgaatcgtcatgattt
agacttgatttgtcgagctcatcaggtatacagtatacgatattttattgttaatttcacacattttcat
ttattggtaggtaggtagataatttaccctgatgtcttttagtttctgtgacacttgaccaaggcagctt
ataaaagaaagaatttaattttgagctcacagattctgtgaccattgtggcatggaaaggggtggcaggc
aggtaggcatggctctggagcagtagctgacaatgtccatcttgaggcaactgccatggggcagagagcg
agaactaagaatggcttcggcttttgaagtctcaaaaatcccaccctaatacttcctaacagttccacct
tctgaggaccaaacattcaaatatatgaactgataacctagggaaggtagtgggggaagggggttagaga
gttggctcagtgatttaatagcgctggcttttcttctagaggacccaggtttgattctcagcatattatg
tgtcagctcaaaactgtctgaaactccagtcccaggagaccagacacctctagcctccaatagcaccagg
catgcaagccatgcagacacacttgtagcaaaacacccatacacataaaataattaactacaaacttaaa
aggtaatatgacttatatgtgaagaacatagatgaggaggcaagactctagatctcgcaagtcagtacca
ggcagtgttagcttaggcatctcactctgagtgattatgtttcaaaagagaacattctgtagtacagtga
gctaactgcagccccacagtgctttgctggtttccaggtggtctgttctctttgtggcctctgtgtgata
tggtaataatctgatactacccaaatgtgtcagtattgtttcttacaaaaatgaactgtgatatgagaat
ttttctgagacagttgtggaatgcaattataaactggttaataaatattttagcttaaaaaaaaagtcat
tattgatagtgtaaaagagtgtccactgttcaaagtgttcatgatccgtaaaccataattggtttataat
atattggctgaaaatattttttacctcttattttaattttgtttttaaagtgtgtattttattttctatg
tgtaaatgctttgctttcatgtatgtgcaggcactataggcttgcctagtgcctacagcggtcagatgaa
cgtgccagatctcctggaacctagagtttcagatggttgtgagctagtgtgagtgtttgagaactgaatt
caagtccatctgagagagctacaggtgctcttaactgctgagctgcgtcttcagctctgttggcctctct
tctttgttggtttggttttgtagattaaaaaaagttccatgtgtgtatgtggattgtgtgcaagtggaag
attgattttgtttcgttttctaccatattgggaataaggccatggtctcagcacctgatactgagaggct
attgttgttgtctggccgtcgcttcattagcctgcattagcaatgtggttcaaatgactcactaataagt
gcttttcttattttctttgacaggtggtggaagacggatatgaattttttgctaaacgacagttggtaac
cttattttctgccccaaattactgtggcgagtttgacaatgctggtggtatgatgagtgtggatgagact
ttgatgtgttcattccaggtaagacaggaaatgaaaatgtctgtctgtcttcccttcccttctttccttc
ccctttctcttttctttgagataggatctcccactctagcctgggctgaccttgaatttgcacttgctgc
tttcagaaactttaggttgcaccattcttatttcagatgtttaaactattaatccttctgttatatactg
gacagtgaataattactaaaggaatggaaatggcaagctggaaaggtggctcagtgagtaaagtgtttgc
tgtggaaggttgcagacctgagtctggatcttcagtacccatggagcagtggcaagtgcctgtaatgtca
ggctaaggaggtagagacaggagggtaccagggtgttgctgcctagccaccttaacccgttaatggactt
caggtttatcaaagcagctgaggaagacacccaacattggctgacctttctgtcatacacaggtatcctc
ctacacagaaaagaatgaatgcagtggataaaataggtttctgtttccttttgcaagtttagtgtatgag
ataggcattggggaagaaagagatgaaaataagataagagtgtttacctaaaagggggaatttccagtca
tctttagtacacttacagaaacatgcacatttcaaaggattcgctcttgctttgttgtgctgcttgttca
aaaccattcttaattgctggttggtaaagctcacttctgccacatgtacacattgggattgcattcagaa
ctctcattacctcagaactctctgaggattcatacattcctcatataaaatggcacagtatttacatata
acccatacatagccttcctttccgtaagatttaagtgatttctaaattgtttacaatgcctgctgtttaa
attagaatcatgttgttggcctggtggtagtaatgcattttaatcccagtacttgggaggcagagacagg
cagatatgtatgagctcaaggtcagctggatctacagagtgggttccaggacagccatggctatatagag
aaccctgaggggaaaaaacaacaaaaaacaaaaccaaaaaaaattcatgatgtttagagaaggccagaaa
tgaccatagacgttcaatccagaaacttctttttctgaattttactgtaatatagttcaatccaacagct
gaactaagaggttgcagaccactggtgtgtacaactgtcatcaaggtttcccttgtattaaactatatcc
atcaactacagttgaatgtcaaattgttggtatcttcattatatattaagggttctaaccaggtcttgtc
cttcagcagttgtcataaggagtcagaataatgttatttcttttacttggatactctagtatgtagagta
gttttaaggaactagagagatagcttagcagctaaaaccactagctgctcttcattcagcagacagatgt
tcaattcccaacacccacatggcagctaacaattgtaactccagttcctgggaatccaacaccttcacac
agacataaatgtaggcaaaacacccgtgcacataaagttaaaaataaattattttttttaaactacataa
tttttccatacttttttaataagtaaaactcttatttgtgctcactataattagtgtaataaaagtgtca
actgtttatgcatgatgtggttgcatatgtctttaatcccagcagtcaggaaacagagacaggcacttat
ctttgagttcaaggcttacctgcattatatagcaagtttgaagtcaacaagatgacatactcagaccctg
tctttaagaaacaaaatgtagccaggcagaggcaggcggatttctgagttcaaggccagcctggtctaca
aagtgagttccaggacagccagggctaaaataaaaaacaaacaaacaaaaaagaagcaaaatgttgcaag
ggcgtggtggaacagtttaaaacttaatactaatgatggaaatgcaaattactgcagctgccttggaaca
cagtttggcagttaattaaagggttaaacattgtgtcattagatgactgagcacaattagatctatacaa
aatcttttataacattatttataataggtaaaagtggaaacaatcccagagcctagctgaagaattgata
aataaaatgggctatccacccatacaatagaacattttagcaataaaagcggctacagatcctgacagat
agcgcagcatggaagaacctattcacacagatcatgttatttgctaacagtattaatgatgctaatagtt
ggcatcagttatatgaaatattgtagactagacaagtccagaaggacataaggtgggtgagtgattgcta
acgcttacgaggaaggagatggagagccagaatgtcatctgactaagaacaaggagttttgggggtttgg
acacaaactactaaaatcagataactgtagtaaatatatactatactaagtaagtaaataaacatactaa
aaactgtacatgttaaatggttaaacatgatgatatggagtgggccatggtggtgcatgcctttgatccc
agcagtcgggagcagatctctgagttcaaagctagcatggtttacagagtgaattttaggacagccagga
ctacacagaggaaccttgttgccggggcaggggtcaggggtagggaggaggtgtgtaggggagacttagt
cttgtggaatataccataaaaaaactttaagtaagacttacctgtatatgaaatatacagtggcttacgc
ctataatctcaacattcaaaatggtgaggcagaaggattgccacaaatttgaggttggcctgactacacc
aacagcgtatctcaaaaaacctgggggaaaggtcagatttagatgagaacctagggtttttgactcagac
cagtattttttcctctctaataaggtgaactagtagagaagattctttaaaacttaaatttaagcctcag
ttatgaggctgtctatctaagctgtgtttctagaatctaaagcaaatgttaagcaagtgttcttccttgg
gggagggcggtgctgcagaccaaatccaggcacttcctattgagctgctcggccaaacttacacttcaat
ctaagaaatgtttgtgcagaattgctaaggtatttttattttcactggacagttgatggcatttattgaa
agaataatattgttaataaacacattatttatttatttatttaaagatattgaaaccatctgaaaagaaa
gctaagtaccagtatggtgggctgaattctggacgtcctgtcactccgcctcgaacagctaatccaccga
agaaaaggtgaagacaggaattctagaaagagaaaccatcagatttgttaaggacatacttcataatata
taagtgtgcactgtaaaaccatccagccattcgacaccctttatgatgtcacacctttaacttaaggaga
cggtaaaggatcttaaatttttttctaatagaaagatgtgctacactgtattgtaataagtatactctgt
tataatattcaacaaaattaaatccaaattcaaaagtatccattaaagttctatcttctcatatcacagt
ttttaaagttgaaaagcatcccagttaaactagccctgttagtgacccagatgaaagcatgaagatccat
ctgtgtaatgtggttttagtggtgcttggttgtttcattattttgagcttgttttgttttgtttgttttt
gctagaataatggcatctacttttcctatttttccctaaacatttttaaaagtgaaaatgggaagagctt
taaagacattcaccaactattcttttcctttacttatctacttaagtaactgttggatcttactaagaaa
acttacccctcattacagtaaaaaggaactttagaggtcgataggttttaaaaatatacaaactatctga
tccattgattttaatcaaacagtttgactgggcaaactttgcagctgataatgagtatttcgctttttac
aaaattgccactgatttggatttgtgcactctaatctttaatttattgatgctctattgtgcagtagcat
ttcatttaagataaggctcatatagtaatatccaaaactagttggtaatgtgattatgtggtactttggc
tttgggttctaattcgcacgaaacaccttttggcatgcttaactttctggtattaccctcacctgcattg
gttttgttttttggggtttttgttgtttgtttgtttttagattcacagaacatgagaatcctttttgaga
agccttggatagctggctctcttctttccctctctctatgtgaaggatgtatttaaatgaacgctggtca
gtgggacatttgtcagctctgaatattgggtgcttcactaataattgccatgtgaatgttgttttgactg
taaggctatgtcactaaagatttttactctgcgttttcataatcaaaggtcatgatgtgtatagacatgc
tttgtagtgaagtatagtagcaataatttctgtatgtgatcaagagtttattgcattatttctttccctg
ttctcttttttttttttttaagggttagcattaacaaatgtcaaggagtagcaaagtcaacaaagatttt
agaaggaggaggaactaagagcatacacagacttatgattctttggatgtgacacttattggatgtgatt
ctaaagtcttttattgaacattgtcaaattcgtacgcttcataggatggacataatgtttatataatgcc
cttcttatgtgttaccatagatgtgtgaaaccttatagcgtccttgaaagtgttaaattgagaactctgt
taacattttatggattgacacattatattactgcaagtaacatttgattttcagcacagtgcaaaagttc
tttaaaatgcatatgtctttttttttctaattccattttgtttaaagcacattttaaatgtagttttctc
atttagtaaaagttgtctaattgatacaacgtctgagtgattattctgtgttgttttgttttacagtgag
atatgtaagcacaagttgacatagactgaagcatagacagtctctgagctgtagccatgttctattaggt
cacacatgcttttatttaatgcgattggataacttacatactagagtaaacgaacaattgtttactcaaa
caattgctaataggattttagatgttatctctgagtaatcaatacttaaggtagctcaagaaaataagcc
ttagtctcaatattagttaatgtacactaatttgtatcttaaactgttttgttttttgtaaatgttcatt
caaattaaaactaggggcgaaaagtaagcaaattagtattggttgttaaaggatagacatttaccatgtt
ggaaaattattcagacctcttaaaactactttacagcttctcatatataagtactcagtacatcatgtgc
tcctaagaatgataacacagattattaattatactagtttactgacaaagtcacaaagacaaacagtaaa
atacaggctacctttaaccccatgattggtgaggcagacagagacaggtgaatctatgacttgaagccta
gactacatgttacagtgtctgtttactagatgaaaagctacaagcaattgcagagaatttgggttcaatt
ctttttttttttttttacacgtattttcctcaaattacatttccaatgctatctcaaaagtcccccacac
cctgggttctattcttaacactcagaaggctgtctgtatggctaagccaaggggatctgctgttttatgc
cctctgagggcaccaggcacacacatggcacacatggtgggtacatagacatacatataggcaaaatcct
catacacataaaaataaaatgtttaaaggatttttttattaagtgttgagcaaataaaatgagttttttg
attgga
Mouse PP1 subunit C (SEQ ID NO: 12):
gtggtggccagaaaatcacagaagaagccagcccagagcctcaaggacacatcaaggtgacttcatttgt
cccaggcccttcttaagccactcaggctgacaggcaatgacaactagcatatgttgatttaaaaaaattg
gaagaaggggttgggatggggtggccttgcatatcccctaatccttgcactttccggaggcagagcagga
ggatctctgagagttggagtccagtctggggtatacagcaagtttctaaccaactagagctacaagtttc
agagacaggtgttagaagctttgaggaccacacagaactcagatctctgcctgcctctgcttgccaaggg
gattaaggcatgcattgctacacttagcgagctgccacgtggatgctaggaattgaacctgggtcctctg
gaagactagacagtgttcttaaccactgagccgtctctccagcctcaattttttttttttgtttgttttt
cagacagggttgctctgtgtagccctggctctcctggcactcactctgtagagcagagtggcctcgagct
ccaagagctacctgccacttcctcccaagtgctgggattaagggcatgggccaccaacgcactaaaaacc
tgttttgttcatatttcctaaagggagtggcatgtggctcagctggtaatgttcacctagcatgcccaaa
gccctgttttctgtctccagcatgacattcagcaggttcagggaccacacattggatttcaacattctgg
gggtgcaggcaagaggatcagtagttgaaggtcaccctctgatagctacaggttcctggacaaagttcag
gctctctaatgtgagcttctttactcacaagagagccagttctctggggtgtggctatggactctggggc
atggctatgaactctggagggaacatgtcccctgtatcatcagcttggtttgtaattttccttcacccct
caccacatggatgggatgacatacatacctgtcacttggcatctagataattatgacacaatgtgtgggc
ataaacattttaaatctttctattgcttccccaggagtgacacacaccctatgtgccaaagtctgctggg
ctccgaaggctccatttagggacttattaaacaaaaggctgttgtttcaggctctgcccagttctagtcc
tcctcctggaatttagcaactgtgtgaccttcagcaaacaacccagcctttccgtgcctaactttatctg
taaaatgggaacggtgggaatatatcatctctaggattgtatcggggattccatcagctgactttgtttc
gttcgagacaagattttgctgttagccttgactggtctggaacttgcctcaaattctcaaagatctgcct
acctctgcctccccagtcctggaaataaagtcctatctaaccatgcccagctacttcatcatattttatt
ttatatgtacgtctgctagaaggtcagaggacaacctgtggtagcttcctctctctccattaggatgtgg
ggcatggaactctaattggttggcaggcatggagggaatctcttgtctgcagagctgtttagatagcctc
ccacaaagagacagattttggctcatactagtaagagtggtgtgtgtttgtctctccatcccacaaaatg
agtctcccgggggagagaaaggctttctctgtcttgctcaacctgctccacccagaatagacactccttg
tgaatcagtcttacatatccctggccctatggtgtccctgggaagctaccctgccctggagggagtccaa
gattccaaaaactcagtgaagaccacattgacctccacagattcacagcagggcttagcatcgaccccag
gatgtcaagaagcctggtgcaccagctgccttggggtgatgaatgaccatttgtgtaatggtgtgcaaaa
ccatagcttgggacaatgagttggtgagtgggcaaagtggagcagagggctgaggagagctggggagaat
cctgaaaccccactgtcgggggctagcatcaggaaaagggctcagatctagatgggtgcatcacacacca
tgctgcggggccacgggaagacagagcttgtagaaaatgagtctctctatggggagacaaccttgcaagg
cattcatggaaaggaatcctcagatccatccctcttgctttgagaaacatgaactggaggtggggctggg
ggagaacattttgtaaattttctggcgtgctactgggaagagataaatgggtcagatgggtgtgcatttt
tcttccataggcagctagcctgcgaggcagcagagttggggacccctcaggtgaaggggtgcccctgggg
agtcaaggaagctagaggcaagcagcagatgaatgcgctgaggcccagagcgcacgcacagcaacctccc
aggaaatgtgcaaattcccattttatctgctccggcacgctcattcaacccagctccggctaaatacagg
ggaaatctcttaaattattatataactttctgagttcaagaaagataattcaaaagataggtagtagcaa
cccttacagaattctcccaacctctctctaaaatgtaaaagagcattagcgcacacaagaagctcacaca
aattacttaatgtgccactcacagtggattaacatttttttggcttatcttgggggcttcttttttccaa
atattgtatcgtagatgctttattgcagtattatgaaattttgccagtgctgggaattgaacccaaggtc
tcacacatactcgacaagcattctcaccacggagccgtatatctagctcttcttgttcaccaatgtgcca
gagtatttgaatccccaacgttccaacctcctcctctttatgattctccacaggcaagcaaccccacccc
agattggctccgaggggtgattcagtgttgaactcccaccccgccatgtcggtcagacttctgtccccaa
gcagcagagccggaaggagctggtgagcctcgcagcgcgcagcagactgacaaagtccaccttcaacttg
cgcgcgccaacttcacaacttccctctcccgccacagggggtctatttcccccgcccgtttggcggaagg
atcccgcgcgcggtggcggagtcgcgctcaaccgtctccgcccgggctccgccccacccccgccctacgc
gctcaaaccccgccccttcccggcgccggccccgcccccttcccgcgcgctctatgggcccgtaaagaag
tcccggccgggccgctgcactcccgcgcgcgcatccgtgcgccgcccgaggctggcctgaggagtcggcg
gccatcttgttcttctcgtggttcccagtggcgagaggaggaggaagcccggagcggagcggggcggctg
cgtgaggaaggaggacgagtgagaccccggggcgagcgggcggcggcgccgctgctgctgctgctgctgc
gggagggtcggcggcgggacggcgatggcggatatcgacaaactcaacatcgacagcatcatccaacggc
tgctggaaggtgagcggcgcgcgggtgggtggcggggcaggcccggccggtgggggatcaggcccagtgg
aggggccggtcttttctcggaagccgctttggatctggggcgcgcatacaagcgcctccgggtttgggga
ccccattattttttatcactcccctgtccttgctttcacatttatcatctggagtccaaggactcatcag
agggacttggcttggcaggacgacgtcccagtgcgatgagagtccgtctcctgcattttcctaccccctc
caccctcccctcaaagccatccatcctccagcgttctttcccccttgaacgtgcatccgcacacggaccc
agtccgggtgacagtgtggctctctctgatcatcagattgttgtgtccctgaggacagggggacgctgcc
gcgttcagcgagcacccgccaggtaggtgctgtgttgttattatcctggaagtgggtttcgcaagagaaa
ctcccaagacctcgctggaccaagaattaacagttaaaagtcaggtttcaaaccccggggtagctaggaa
cctagagagaacctgccgccttccaactgttcaccccgtgggaacccaggatgtccagtggttgaggaga
ggttcaaaaacaaaaacccagctttccttttgtccgacttgggggtttctgccgtgtggtttcctacttt
ggagatttcaaatttgacataggtctggatcctgctaatcacagatcccacaacatccgctgaagctaaa
gaggagaagatccagtaaccttgatttgggctcttgacactagcttgctttggcccaaaatacttttgga
gtggtggacagctgcgcaattctggtggtagatgtcttcaagtaaatcactgacagaaaagggaaaaggt
gtctttttaaaatctgggcatttccccggggggcgggggggtgtttttcaagacagggtttctcagtatg
gtcctggggctgtcctggaactcactctgtagagcaggctggcctcaaactcagaaatccgcctgcctct
gcctccctagggcacggattaaaggcttgggccaccactgcccagagcattgcctcctctttaatgtttt
gtttacatagggtttcttggctttggtttcattaaaacatggtctctccagtcgtggttatcctggcact
ctttgtgtagaccaggctagcctcaaactcacagatccttcttcctctccctccccaattaaaggcattt
ttattaccatgcctccttttattacagggtctctctcaggggccagagctgagttaaccttctgatcctt
ctgcttcgtctcacatactagattttataggccactcagcctgacttatttttttcctatgcagtgtgtg
catacatacagaccaaagcacacgtgaggaggttagagggtagttgggcgtctttgtgtggattctgggg
atcaatctcaggtcaggcttgcaaagcagattttctactgactaaatcatctcaccagcttccctttctc
cctcttttaattatttttttaaattttctgtatgtattttgtttgtgtgcatgtgcgcgcacatgcatgc
accgtacgcatgtcttacccttatgaatgtctgaaaagggcattgggtccttgggtattggagttgcaga
tagttgttagctgtcacatatgggtgctggaaatggagcccagtttatctccaagaacatcaagcactct
tagcggctgaaccatcacttcactcccccagcacccccttttcttgttggtttttttttttttttttttg
tctttgtttttcgagacagggtttctctgtatactctgtatacagagtttctctggaactcactttgtag
accaggctggtctcgaactcagaaatccgccttccgagtgtgggattaaaggcgtgcaccaccacgcccg
gcttcagcaccccctttttaaatgagacagtttcattatgtagcccaggctggtctctgtctcaactatc
cagagtactgggattaaaaactttttaccacccctgtcttctcatagtttcttaatagtttttaagttct
gagactgggtagcattgtgttagcaaaggctggcctggattttcagagatctatctgcctctgtctcctg
agtgctggcattcaaagctgaactaccatgtttggcctggtttattcttttctgttgaacttaacagagt
aagccactgtttaaacacgttgagaatgactggctgggtggtatatgtatttgtttctaacttatggact
tgcttgacggaagccacaggatctgctcaggatgcgtatagttctcaatcagaggacgctggcattgggg
ttgtgatgatttcaaaacctttaaatctagagatagctcagccggttttggctcttgcccttaatcctga
agtcctgatttgatcctctggattgacatgagattcaggagagaaccagactttgagggttgtctccacc
ccacccccaacagctccagcaagcatggccttgcacacatgcatataacagaatggtataaaaaaaataa
gggtttaattctgaaagggggagataagatttgtcccttggagccgggtgcctttaatcccggcacttgg
gaggcagaggcaggccggtttctgagttcgaggccagcctggtctacaaagtgagttctagaacagccag
ggatacacacagaaaccctgtctccaaaaacaaacaaacaaaaaactggaaagctgtcttagggctaaga
gtacacagtactcatctagaggatccaagctcagcttccagcactcatcccaggtagcccagtgcttgct
actccagtaccaaggagtcagacaccagcttctcaaggcacacacatacatggctaacccacacagacat
gattaaaaatacattttaaactgacagtggtggcacatgcctttaatcccagcactgagaaacagagaca
acttaatctctgagttcaaggccagtctggcttacagaagaagtgccagactatccaacgctagctactg
tagtccctggcacttaggtgggggcaggaggtcgaggcagtagcataatgaaatgagttcctaagggtct
agcctgaactacagattctgtgtaaacaggcaggttgggttgggttcctaggcactgcggttctgtggca
ctctgaatgcagtgggtttccctgagtgtcaccctgggtggtcagccacactggcggaagatctgtgtta
gtcctcagcacagtgttagggacatgctagagcagaaaatagactagctgtgtgtgaaggtagcaccctg
ctctgctgttttagtggaaaatgggaattggggtgctttcttcatgaaacctctagtgttttttcttgag
tagcaagtggttttaatttctccacatggcaatgtgcaagaacatgtttctgtaaatgaaggtcattgtc
agcgcatggtgggaagtagctctgctaatacagattgctctccagtcctcgtctctgaaggcatctgact
gccctaccgttctctaaggacttgtgtatgtgtcttttcggtgctcctgagggccagggtcctaaatgaa
agacaggtgagggtgatgggcttgtgggtctggaagcagatcctagactgagcagagggcacacaacaca
gcaagatcatcattaacgggcatcctgccctccctccacaccaacaggtatatgtccccatactttacct
ggcaggcagggcaggaccctctcctcaatctgtagatgtttgagtcttaaaaaaacttaatttgggtgag
acatcggtggcccaaacctttaatcccaggactttggaggcagagtgatgtctgagtttgagaatagcat
gatctatagccaggcatagtggtgcctttaatcccagcactcaggagacagaggcaggggcaggcggatt
tctgagttcgaggccagcctggtctacagagtgagttccaggacagccagggctatacagagaaaccctg
tctccaaaggaaaaaaaaaaaagagagaatagcatgatctacagagatagttccaggacagctagggcta
cacagagaaaccatctccttcctcctccaccctccaaaaacctccctttgtcaggtatggctcctagagc
cttcactgccccttcacttttgcaaggccaggctgcaggtcagtcctaggcaggtgggacacacaggaca
gccacctctcaaaggaaagcagctggcctgtgtgatcccacagtggctgggttggggtggtcagtgcact
ttaatatctgagggaaaagagctatgtgtgttttttactgctttgattcaggctgggtcacaggaagaaa
ggttgggtgggaactgcctcatagctgaggaagccccacaggcccactgaagtaaagcaaatcccggttc
cctgagctagaattctgggagggatttttgtcttcagtcttggtcacagttgtatcgggagtgtgaaggc
aaggtactgattctgatgatgtttcccacctctgtgtcagggttgctgacatccttgtttttgaagggtg
tattttaggacttacaggaagtagaagctcatggggtccaatgtgcaaggcactgatgctgagatgcggc
attcagctgtggaaagattctcaggctgccctccaacttccttcacagctgagatgaccttgaactctgc
tgatcctcctgcctcccggtgaaatacagtgttcaaatggcttagtgttcattatagaaaacaagatacg
tccttgggacgttttttggttggagcttttttagccacatcctggttgcccacgtagaggagatcagtgt
tgtcatgcatgacagcagcagtgccccagcgaggtgggagggtagtgggagacagtggggagagccatgc
acttttgctgttggggggaaaaaggagttgttgaggagtttctttagcttgcatgttttgtagaaaataa
ttcagtttgcaagaaagtgttcagtgtggcgagtactaagtttgtttggtttgagatctttttctacatg
tttttaaaataccttgaaggtagtctttcaccaaggaatgacttaaatttgttttttatttgttgaaaaa
aaaggtctcatcactatgtagatcaggctggccttggactcagatctgcctacctggggctagatgtatg
cttggccttggcagtggctgcttaggtaaggttgtggtcagacagcaagggctgctgactcgccccttct
gtgcagctcttagctccaaatcacattgtaattcatctagggctttgtcctgtccttttattacttattt
tttaattgagatggagtttcctgtgcctcggctggccaggagggtctgtgtagatgaggaccttgaactc
ccgatcctcttggtacacagcccagttttaatgctggggcagaaccccagggcttggtgcattacgagcc
ttgagggtgctaccaaccctgtggaggcttttctatccctcagtacatctccttggcttccttctgtcat
gctgtcttgcttgttgctagactgtggaaagttactgctgcctagagaggtgctagggctgcagactacc
agcagcagcagtagactaccagcagcagcagtaacctgattaagctcagggtttatgaactttggggttt
gttgtcagtgagatttttagtgaaatttctctgtgtgctgatgttagtgatgttccaggtaagtgcagta
tctacagaacagcctttgttgttagctgccagccgacctaaataaatgtcccttttctaggtacacacaa
gagcctcattgggaacagcaggctgacccagcaatagtaagagccagagaataaggttagatggacaggt
cctgggtgttaattgtgggaaagtcatattgcttgatactgagagctaaagtagatcttgccaatttggc
agctttaagacatacccaatgctcactggcaaaaaaggcttgtagtcctatgcttattttgacaaccact
ccccccctcccccccccaatgccctacccctgacttttgaatcaaggttttcttggggctggttggcaac
caggtgttgccagccattcaggaagtataatgtattcttggggaaggggacagcctcagttgaagagagg
tctgggaagtcctgaagggtgagaagttgttgccagctgtggaatgtgctatgtcagtgaatgctacata
gacagggtgttgttagctagagaagaggggtgaagctgcaggaggatcataaatttcagggttagactgg
gctacataatgagacttggtgtcaaacagttggttgtggtgacatacagctttaatcctattatcgaagt
ggcaggcagatccctgagttgaagaccggccagggctacataggaagaccttgtttcaaaaatcttttaa
aaaaattgcactcaaactgtctaaaaatggcagtggatgttttcagaatcaaggaatattagcaacaccc
agctagctctctgcagtagaaccctgtatgtatgtaacaaagcaagcctgtgggctgtaccaggagaagc
cagatacgaagattatcaccaaagacacaagagtgggccactcgtgcacaaatgcagagctctcctgaag
aaaattcctgttgagaccagaagcttccaggagttcccttctaaccaaaaataagcctcctgctgggcgg
catgcccaggtttttggccggtgaggcaggaatcctcctgtctctcctccgagtgctgcttgtcttgtct
ggctccagtggcaaaggaaagcctttgttgcttagctgctgtcctgccttccctcaaagactcactgcag
ggctacctccatctcagaattggccatgcctgcttagcctaggctgcatagccccagaggggactgatga
gaggctcggtggataaagtgtgctgcataagcctgacatctgagtttaattcctggaaccagcaaatggg
aagaagagagccaactccacaaatttgtcctctgacctactcaccctcaaccccaacccccataaaattt
aaggggaggactggggaagatagttcagcctgtaaaattgctgcagaaacaaggccctgtatttgacccc
accagaaacagtttggtttggtttatttgagacagggtttatctccatagccctggctgtcttggaactc
tccatgtagtgctggggttaaaggcatgtgtcactaccgccaggctcagaacctattttctaaggtgtct
gttaccccagtactaggggatggagaggcgggatcctagtgtagccaaagaatgaaatagaaaaaaaaag
acgagagtacacccgatgtcaattcccagtggccacatcttttgtcttacagcccagttatatttggggg
ggactgaagctgctcagaggggtccattccttgctggctccctgtcctatctcataatgttttttcctgc
tttgagggcccatccttttcttgtcttttccctgaaaaactaatgttgctttgcataccatttcatccct
gatcagcttgttccttcggagaagcggggatgtctaaagtcgtacaggctgaagggcagcttccctgagg
ctctggtttccaagcatgggtacagctttgcagccttgagtggactctgcttggtccatagcaggcctct
cttgggagtgccattgctctgctgctacagggtcaaaagctgtgtttgcctgtagacttttctgctctta
cctagctgtcaggacagatgattttatgaccgagcctgcaattggacaaaatttaagctagtgagatcag
tcagatttaggattctgctaaaagcaaatctgttgactggtttatgatagacagatgcagtcttaaatgg
gctgcttagcctgggtctgctgaactcaagtgaagtgcctgcgtcctttgtcttctgtacaaaagagcag
agactcagattctagctcactgaaggtggtcaggcccctgtgccaggcagccttgcggtaagcagggcct
gcggcagccgttccctgaagctgctgccacttgagagctgtggggagttcagatccttggcagttggagt
tgttcttcagtctgcataatgattatctgatctcgagtaaccggaacccacacagatgtctccactgctg
tcaccctcgcctcctgaggagagtgggctgtggccagaccttaccgaggccgtgaacagagagggctcag
gagctgccatgactggtgctaggctgggagttgttctctttggatgcctgggaggcctttttcttaatag
gcactgacttaatgctggtgtcctatttggatatgaatgccagaggagaacctcagggtgtgtcattaat
tcttcaagggtgatttggctttgggttttttttttttccccttttagctaggctttctcactgactcact
aggtagattaggctgcctggttggctttccagatgtgtctgtttccctctccagcactgggattctagat
gtgtgccatcaagcctggctccccatgccccactccctcttctgtgagttctggaacttaaacataagtt
ctaatgcttgcaaggcagtcttttatcagctgggccatctatctagcttctaggacatttcttaaaattg
atttatgtgtctggtggttgtgcctgcatgtatgttagtgcagtctcatcggaggccagaggggtattaa
tagatccctagaactagagttagagttgtgactgactgacacgttgctgtcggactgaacctgggttctc
tgcaggagcagtcagtgctcttaaccagtgagctcagcagcattttaaagtgaaatgcagacaacacgca
gaaattagggaccatggagttgcacttggtcctgaaacatcattgctggagacatgtcctgaacatttct
gaggacatacttggtgatatgactgtgacctttcccactgcaaacttgagtccattctaaaagtagagag
gcctggaagcagaagggaccgtcttggtacagggcacgagtgtgtcctagggactctgaaacggccagat
acgggcagggtcagggcaaccttggagctgctgtaaaggagcctaacatagcttgaagtatacaactgac
attccagatctcagttgagaccatactggccagccagaaaactgatttcagttacatttggccaactcct
atctaacaggtgtttattatgagaaaaaaaatattcaatatcacaccacgccacccttctcaagttaaca
ttttttctaagattcatttttgttattgacgtgtctgtgcatttgagtgcaggcggcagccttggccaga
agctacaagcagtaattagttagctgcttgccgtgggtgtagctgctcttagctgctgctgtcctgaatt
gaacatttccatgtgaggctgtgtcctgactgctgtttctctccacagtgagagggtccaagccaggcaa
gaatgtccagctccaggagaacgagatccgaggactctgcctgaagtctcgggagatcttcctcagtcag
cctatccttttagaacttgaagcaccactcaagatatgtggtgagtgtgggactcccgttactcagctgg
gggcacactggtggcctttgatgttctctgaaggagcatgggcatgggggggtgcggtggggggcctggt
ctagtttggctcactctgctgtaggagcaggtacagaccgtcctctagagtcgcacagtctaggtgggta
ttgcatctctgactcagaataaactgtgtgtttagagcgaggagaccttggctttgaccttgcagaagtt
gaaaagccatggccaaggtgggtgtcttgaatgtaagtgttgtgagggggcccgtctcactggacagtga
caaaggatgtgttcacataaatccctgaagctgagctcaggggactgcagtacttggcaggaaacaggtg
agacaggatactctttaccacaaataaattaattagttgtatctggggggagctctggacctgtgcattt
aaattttacatggtcagttgaactaaatatgctacaaccgggaaattcgtgttggcggtgaacgttgtag
atgccattgtcatatgagatgagtgtctgaggggcgtcttgccaaggttttgtctgtttgctgatttgtg
acactgatgttctcacccttaggtgacatccacgggcagtactatgatttgctccgtctgtttgaatacg
gtggctttcctccagagagcaactatttgtttctcggggactatgtggacaggggcaagcagtccctgga
gacaatctgcctcttgctggcctacaaaatcaagtatccggagaacttctttcttctcagagggaaccac
gagtgcgccagcatcaataggatctacggattttatgatgagtgtaagtactttgcctctgaggaaccag
ggctagaaggcagcgggtagcaggcctgggagttggagctgctgcagcatacctacccacgtcctggttt
agggagagcagcctgacctgggtgctgatggggttggacggaaatagtcttgatggcacacggctttgct
cttagtgcagaccccagagcttgctccatctgctctgcttttcacagctttggttagactggttctggat
agagagagcatctgctgaaagcatgggtcagtggcttgtgagttgagagcagcaccacagttgcaggcag
tgctgctctgctgtccagccttatatgtgggtggatcttgcagtgaacagcttggttttgaaatggatgt
tgtagttttcttgtagtgcagctttatttaggaaagggacaataacttttctggaagatgcataatggag
aaaaaacgctttaaaaactacattttgatttagtgtgtctgtctttgctggagccagagtgtagagtcag
ctgtctccaccctgaccttggttgccaggcttgcacctttccttctcagccccgcgctggctggctggct
ggcttccttcctttccctttctttctttctttctttctttctttctttctttctttctttctttctttct
ttctttctttgtttttgtttttggggtttctctgtgtatccctggctctcctggaactcactctgtagat
caagctggccttgaaatccacccacctctgccttctggagtgctaggattcaaggtgtgccacatcacct
ccaccctcccccttctcctttttgtttttcctcagtttttgtttgtttcgagacagggtttctctgtata
gccctggctgtactggaactcactctgtagaccaggttggcctcgaactcagaaatccgcctgcctctgc
ctcccaagtgctgggattaaaggcatgcgctaccatggcccggctttgtttgtttttttatttaaagtat
gtgagtacactgtggttgtcttcagatgccccagaagagggcatcagatcccattacagatggttgtgag
ccaccatgtggttggtgggaattgcactcaggacctctggaagagcagtcagtactcttaactgctgagc
catctctccagcccccaattttattttaaaataatactttgtacatattctcatatttgaggttatctca
ttttacttttttttctgaagtgtagaccacaaccttggctccagttgcagcttcctgaccttggtggggg
tggggggtgcccaccccacacagccctggattgtttagtagaactgcactttgaactcatcattaattaa
aaatgattccaaagccttagaaagatttggtgtgggaaaaagaactggcttgggggagggctttcatggt
ttcacacaaacataaaatggcaggagttttgagactctgggctgtttccgccagaaaagggtttggactg
gattggcaggtgcttaagggagtccattggtttagcacttgctaatatggggcacacacacacacatgca
gcagcttgtagttagggctgcccctgttttctagattgctagagcgagcttgagttcagcatggagtctt
ctcacctcagacaacacagagttttaatccagccctcagctaaggcatgagtgacattgggctcagcaga
gaatgagagggaccggatggattttggagtggagtcaaggccatctggcctcagtttccgcattgaagtt
cttactattagctattggtaattggaggtgttgactgtgttcattgaagtttccagaattatccagggaa
tctgtggcctccccagggctgaaatgcaagtttcctcggaaggatctgaatgctgggtgctggtggtggg
tggtctgcccagcttagaaggcgtggcttaggaaggctgttgctaggtgccgctgaactctaaaaggcat
accgtggccctttagatgtccctgtgttctctgttaaagaggacagagtcctagtgtagtggcaaactca
acatactgctactcaagtgtgagatggcccaggagcagaggctgccctgtggtgcgcagtccttggccag
ttagagaatgtaggaggggctacggcttatgaactgtgaactccaaccatagttttcatacagtggtttt
aaagggcacacgctctgacaccccgttataaccctggtgcttaagagggagtatattgagtgtagactgt
taggaccaagcaccggatgtgcccagattgagctttctgattgcccagagtccctggccctcctgaagtc
agggtggggctcctgtctggctgcagcagcttcacagctaaggcagctgaaggaagagctgacaacacag
gaggacgtggcggccttcagaagtagaagttccctcaaggtcaaggttccacaaaccagatgaggtttca
agtagagtgggtgctgtgcgctaatgtggctggctgaggtgccctgtctggagctctggggacgcgtttg
gttctggatgagtcctgtaccccagccctgtgggcctgtgggtgcccccatgtttcagttgcttgaaagg
tggccttagagagcaaaggtgaagagcagtagggcgactgtctgcgttctcacaggtaaaagaagataca
acattaagctgtggaaaacgttcacagactgttttaactgcttgccgatagcagccatcgtggacgagaa
gatattctgctgtcatggaggtatgtgacatggaggtgttgccttcacctaggcagctctaccccttctg
aaaagtcagctgggcacggtgaccctggccaggccactgtggtgccaaggcttcttcacagtgatacagc
ccggtcatttggatcaagtgtgaggaagcaggggttataggttgggaagagaacgagactttaaatacag
gacgagccctagtgccctcttctcagccatgtgtctgcctgaaccctgactgtgctcttcccaaaagctc
tgctccgttcctcagctgttctgtgtgtgaggtaataaaggcattgcaaagatgtggaggtttgggggtg
attggaaatgaagcagctatccaaaaacccacataatgtattaaggcgtagtctctatgccctcttggat
cgggtacttagagaacaagactgttagatctgttgagtgacatttggtggtccatggaccacaggctcgg
aaggagctgcctcacagctgagcattgtcgtgcctgtgcccggccattgcatctggtgatggtggcaccc
tctgtgttcccttcctgccttctattgggagaagtgtggattcagtggttgggtgttgcggggctctgtg
ttgaggtgactgtctctgaccagcacaccaacagcagtacagtacatttcgtgcattgtttcccccaaaa
ccaagttaaaaacccacaggtttcaccgcagcatggcagtgccagcctcctcagcaggaggggctggcca
catgctcaggccaatgctgtctgcacttcagggggtttcctttctcgttgaataggtttatcaccagatc
ttcaatctatggagcagattcggcgaattatgagaccaactgatgtaccagatcaaggtcttctttgtga
tcttttgtggtctgaccccgataaagatgtcttaggctggggtgaaaatgacagaggagtgtccttcaca
tttggtgcagaagtggttgcaaaatttctccataagcatgatttggatcttatatgtagagcccatcagg
tactgactttgactttacatgtacacttagagcatgggtgtgagccgaggctccttgttgattttggtgg
gtaggtatggcttgccacacagtgtgcctccggggcgagcggcagcaggccgagctcagtggggaaggag
aggggcagctcgtgccccagctgctcacactgacacggtctctccacaggtggttgaagatggctatgag
ttttttgcaaagaggcagttagtcactctgttttctgcacccaactactgtggcgagtttgacaatgcag
gcgccatgatgagtgtggatgagaccctcatgtgttccttccaggtgagactgtggccacttttcatgtg
cttgcacctctgctgagcaggctgccctttcttttttaggaatatttatcccatggaaaaagtgtgtggg
ctcttcctaaagaaaggaaaaataatacggaaccttttcccaggattgacgttaggtgtagttgaattct
ttaagtgggtggaagcaagcggtgtgttacagtgtcacttatagtcagtgcttttggtgagaagagggag
ttggggtgcctctggctggcctggaacacaccctgtagaccaggctggccatgaatgtaagagagattta
cttgcactctgcctcccaagtgccaacacatcagcttgtgccatgatatcgtctcctgtgctggccccgg
ttcttacatttcctccctatagtgggcttgatccatttcaagctcacgcagttgttaatgttactgtagc
tgtggaagtcccgaggcaggcctcagcactccccagagactgcagactccagtgccttccacagagcttc
aaggttgtgggaagctctagacttgagtttgtcacttggccccagcagaaactgttttccagatcacata
ttctggctgtatgtcatggcacttctccaccaaaatgctcagggaccagtcggtccagggtatgttctag
agggcttttcagcatttagggggcagcagcattaggcagaacttctagatggagatctgaacagagctga
ggtccaggtatagctctcctaaggttcttctttctttcctctttacagattttaaagcctgcagagaaaa
agaagcccaacgccacgagacctgtcacaccaccacggggtatgatcacaaagcaagcaaagaaatagat
gtcacttgacactgcctggttgggacttgtaacatagcgttcataaccttcctttttaaactgtgatgtg
ctggtcagcttgcccaggtagacctgtctgtcgggccctcctccatttgattactgctggcacttgctgg
ttatagcagcaagccaagcacttcattctcaagagagcattttgttttgaacctctgttccctttgtgga
cagctctgatgatggtgttaagctgtacaccctggcaggttatcctgtctgaggagaaagtgtacaattg
atctttttttaatttagtataagtcatgaataatgtaaatgcctgttttctttaggatataaagagagcc
ttagagtgcgtgagtctctacatgtaattgtcataaatgcattctgttgatacaaaccactgtgaacaat
tttttttccagtttgtttgaaagggactgctttccctcattgtcttgtcatgtacaaactagtgtctgca
gctgtggcagcaggagtgacctgcctgccgccagccctgcccagactatctgaagcacactccttcccac
tgcacatttaataatgattaaagccattcttttcaatgtctgtgattccttcctaaagccaaagtttctg
ttggactgtatggcacgccctggggatgaggtggccagggcatcgaggctgcgtgcacaggccgcctccc
tccgtggggcctcagaagcaggttattttaactagcaatagtggtatagtgctgagtaagctattaatga
tggaagttaatgacactttgtacagttcccatatagtctattcactgagtgatctttttacagttggatc
aggcctgaacccgtccattcagaaagcttcaaattatagaaacaacactgtcctatacgagtgaccgata
atgctttctttggctacattctttattctgcggtgacattgaggcttataaatcaaaaggaactaacttg
ccgtccaccggtttatacagaactcacagtatctatgacttttttaaactacgacctgttaaatgaatct
gtttgcacagatgcccgtgtacaatgccatgtgctgagaatggtttcagacttattaaatgcaagcttgt
taacttg

In certain embodiments, the PP1 comprises one or more of the above subunits, or a sequence that has at least 85, 90, 95, 96, 97 or 98 sequence identity to one of the above listed subunit sequences. For example, the PP1 may comprises one, two, three or more of the the above sequences of human subunits A, B and/or C, or mouse subnits A, B and/or C, or a sequence that has at least 85, 90, 95, 96, 97 or 98 sequence identity to one of the above listed subunit sequences.

### MAP/ERK Pathway

The MAPK/ERK pathway (also known as the Ras-Raf-MEK-ERK pathway) is a chain of proteins in the cell that communicates a signal from a receptor on the surface of the cell to the DNA in the nucleus of the cell.

The signal starts when a signaling molecule binds to the receptor on the cell surface and ends when the DNA in the nucleus expresses a protein and produces some change in the cell, such as cell division. The pathway includes many proteins, including MAPK (mitogen-activated protein kinases, originally called ERK, extracellular signal-regulated kinases), which communicate by adding phosphate groups to a neighboring protein, which acts as an “on” or “off” switch.

The term “ERK” refers to any human ERK1 or ERK2 gene or protein. ERK1 is known by several names including, for example, mitogen-activated protein kinase 3, extracellular signal-regulated kinase 1, insulin-stimulated MAP2 kinase, MAP kinase 1, MAPK 1, p44-ERK1, ERT2, p44-MAPK or microtubule-associated protein 2 kinase.

ERK2 is known by several names including, for example, mitogen-activated protein kinase 1, extracellular signal-regulated kinase 2, mitogen-activated protein kinase 2, MAP kinase 2, MAPK 2, p42-MAPK, or ERT1.

In certain embodiments, ERK1 comprises the following amino acid sequence (NCBI Accession No: P27361.4 (SEQ ID NO: 13), incorporated herein by reference in its entirety):

| | |
|---|---|
| 1 | MAAAAAQGGG GGEPRRTEGV GPGVPGEVEM VKGQPFDVGP RYTQLQYIGE GAYGMVSSAY |
| 61 | DHVRKTRVAI KKISPFEHQT YCQRTLREIQ ILLRFRHENV IGIRDILRAS TLEAMRDVYI |
| 121 | VQDLMETDLY KLLKSQQLSN DHICYFLYQI LRGLKYIHSA NVLHRDLKPS NLLINTTCDL |
| 181 | KICDFGLARI ADPEHDHTGF LTEYVATRWY RAPEIMLNSK GYTKSIDIWS VGCILAEMLS |
| 241 | NRPIFPGKHY LDQLNHILGI LGSPSQEDLN CIINMKARNY LQSLPSKTKV AWAKLFPKSD |
| 301 | SKALDLLDRM LTFNPNKRIT VEEALAHPYL EQYYDPTDEP VAEEPFTFAM ELDDLPKERL |
| 361 | KELIFQETAR FQPGVLEAP |

In certain embodiments, the ERK1 comprises the following nucleotide sequence (NCBI Accession No: X60188.1 (SEQ ID NO: 14), incorporated herein by reference in its entirety):

| | |
|---|---|
| 1 | cgttcctcgg cgccgccggg gccccagagg gcagcggcag caacagcagc agcagcagca |
| 61 | gcgggagtgg agatggcggc ggcggcggct caggggggcg ggggcgggga gccccgtaga |
| 121 | accgaggggg tcggcccggg ggtcccgggg gaggtggaga tggtgaaggg gcagccgttc |
| 181 | gacgtgggcc cgcgctacac gcagttgcag tacatcggcg agggcgcgta cggcatggtc |
| 241 | agctcggcct atgaccacgt gcgcaagact cgcgtggcca tcaagaagat cagccccttc |
| 301 | gaacatcaga cctactgcca gcgcacgctc cgggagatcc agatcctgct gcgcttccgc |
| 361 | catgagaatg tcatcggcat ccgagacatt ctgcgggcgt ccaccctgga agccatgaga |
| 421 | gatgtctaca ttgtgcagga cctgatggag actgacctgt acaagttgct gaaaagccag |
| 481 | cagctgagca atgaccatat ctgctacttc ctctaccaga tcctgcgggg cctcaagtac |
| 541 | atccactccg ccaacgtgct ccaccgagat ctaaagccct ccaacctgct cagcaacacc |
| 601 | acctgcgacc ttaagattty tgatttcggc ctggcccgga ttgccgatcc tgagcatgac |
| 661 | cacaccggct tcctgacgga gtatgtggct acgcgctggt accgggcccc agagatcatg |
| 721 | ctgaactcca agggctatac caagtccatc gacatctggt ctgtgggctg cattctggct |
| 781 | gagatgctct ctaaccggcc catcttccct ggcaagcact acctggatca gctcaaccac |
| 841 | attctgggca tcctgggctc cccatcccag gaggacctga attgtatcat caacatgaag |
| 901 | gcccgaaact acctacagtc tctgccctcc aagaccaagg tggcttgggc caagcttttc |
| 961 | cccaagtcag actccaaagc ccttgacctg ctggaccgga tcttaacctt taaccccaat |
| 1021 | aaacggatca cagtggagga agcgctggct cacccctacc tggagcagta ctatgacccg |
| 1081 | acggatgagc cagtggccga ggagcccttc accttcgcca tggagctgga tgacctacct |
| 1141 | aaggagcggc tgaaggagct catcttccag gagacagcac gcttccagcc cggagtgctg |
| 1201 | gaggccccct agcccagaca gacatctctg caccctgggg cctggacctg cctcctgcct |
| 1261 | gcccctctcc cgccagactg ttagaaaatg gacactgtgc ccagcccgga ccttggcagc |
| 1321 | ccaggccggg gtggagcatg ggcctggcca cctctctcct ttgctgaggc ctccagcttc |
| 1381 | aggcaggcca aggccttctc ctccccaccc gccctcccca cggggcctcg ggagctcagg |
| 1441 | tcgccccagt tcaatctccc gctgctgctg ctgctgcgcc cttaccttcc ccagcgtccc |
| 1501 | agtctctggc agttctggaa tggaagggtt ctggctgccc caacctgctg aagggcagag |
| 1561 | gtggagggtg gggggcgctg agtagggact cagggccatg cctgcccccc tcatctcatt |
| 1621 | caaaccccac cctagtttcc ctgaaggaac attccttagt ctcaagggct agcatccctg |
| 1681 | aggagccagg ccgggccgaa tcccctccct gtcaaagctg tcacttcgcg tgccctcgct |
| 1741 | gcttctgtgt gtggtgagca gaagtggagc tggggggcgt ggagagcccg gcgcccctgc |
| 1801 | cacctccctg acccgtctaa tatataaata tagagatgtg tctatggctg aaaaaaaaaa |
| 1861 | aaaaaa |

In certain embodiments, ERK2 comprises the following amino acid sequence (NCBI Accession No: P28482.3 (SEQ ID NO: 15), incorporated herein by reference in its entirety):

| | |
|---|---|
| 1 | MAAAAAAGAG PEMVRGQVFD VGPRYTNLSY IGEGAYGMVC SAYDNVNKVR VAIKKISPFE |
| 61 | HQTYCQRTLR EIKILLRFRH ENIIGINDII RAPTIEQMKD VYIVQDLMET DLYKLLKTQH |
| 121 | LSNDHICYFL YQILRGLKYI HSANVLHRDL KPSNLLLNTT CDLKICDFGL ARVADPDHDH |
| 181 | TGFLTEYVAT RWYRAPEIML NSKGYTKSID IWSVGCILAE MLSNRPIFPG KHYLDQLNHI |
| 241 | LGILGSPSQE DLNCIINLKA RNYLLSLPHK NKVPWNRLFP NADSKALDLL DKMLTFNPHK |
| 301 | RIEVEQALAH PYLEQYYDPS DEPIAEAPFK FDMELDDLPK EKLKELIFEE TARFQPGYRS |

In certain embodiments, the ERK2 comprises the following nucleotide sequence (NCBI Accession No: NM 138957.3 (SEQ ID NO: 16), incorporated herein by reference in its entirety):

| | |
|---|---|
| 1 | gcccctccct ccgcccgccc gccggcccgc ccgtcagtct ggcaggcagg caggcaatcg |
| 61 | gtccgagtgg ctgtcggctc ttcagctctc ccgctcggcg tcttccttcc tcctcccggt |
| 121 | cagcgtcggc ggctgcaccg gcggcggcgc agtccctgcg ggaggggcga caagagctga |
| 181 | gcggcggccg ccgagcgtcg agctcagcgc ggcggaggcg gcggcggccc ggcagccaac |
| 241 | atggcggcgg cggcggcggc gggcgcgggc ccggagatgg tccgcgggca ggtgttcgac |
| 301 | gtggggccgc gctacaccaa cctctcgtac atcggcgagg gcgcctacgg catggtgtgc |
| 361 | tctgcttatg ataatgtcaa caaagttcga gtagctatca agaaaatcag cccctttgag |
| 421 | caccagacct actgccagag aaccctgagg gagataaaaa tcttactgcg cttcagacat |
| 481 | gagaacatca ttggaatcaa tgacattatt cgagcaccaa ccatcgagca aatgaaagat |
| 541 | gtatatatag tacaggacct catggaaaca gatctttaca agctcttgaa gacacaacac |
| 601 | ctcagcaatg accatatctg ctattttctc taccagatcc tcagagggtt aaaatatatc |
| 661 | cattcagcta acgttctgca ccgtgacctc aagccttcca acctgctgct caacaccacc |
| 721 | tgtgatctca agatctgtga ctttggcctg gcccgtgttg cagatccaga ccatgatcac |
| 781 | acagggttcc tgacagaata tgtggccaca cgttggtaca gggctccaga aattatgttg |
| 841 | aattccaagg gctacaccaa gtccattgat atttggtctg taggctgcat tctggcagaa |
| 901 | atgctttcta acaggcccat ctttccaggg aagcattatc ttgaccagct gaaccacatt |
| 961 | ttgggtattc ttggatcccc atcacaagaa gacctgaatt gtataataaa tttaaaagct |
| 1021 | aggaactatt tgctttctct tccacacaaa aataaggtgc catggaacag gctgttccca |
| 1081 | aatgctgact ccaaagctct ggacttattg gacaaaatgt tgacattcaa cccacacaag |
| 1141 | aggattgaag tagaacaggc tctggcccac ccatatctgg agcagtatta cgacccgagt |
| 1201 | gacgagccca tcgccgaagc accattcaag ttcgacatgg aattggatga cttgcctaag |
| 1261 | gaaaagctca aagaactaat ttttgaagag actgctagat tccagccagg atacagatct |
| 1321 | taaatttgtc aggtacctgg agtttaatac agtgagctct agcaagggag gcgctgcctt |
| 1381 | ttgtttctag aatattatgt tcctcaaggt ccattatttt gtattctttt ccaagctcct |
| 1441 | tattggaagg tattttttta aatttagaat taaaaattat ttagaaagtt acatataaaa |
| 1501 | aaaaaaaaaa aaaa |

### Ehlers-Danlos Syndromes (EDSs)

Ehlers—Danlos syndromes (EDSs) are a group of genetic connective tissue disorders. Symptoms may include loose joints, stretchy skin, and abnormal scar formation. These can be noticed at birth or in early childhood. Complications may include aortic dissection, joint dislocations, scoliosis, chronic pain, or early osteoarthritis.

EDSs are due to a mutation in one of more than a dozen different genes. The specific gene affected determines the specific EDS. Some cases result from a new mutation occurring during early development, while others are inherited in an autosomal dominant or recessive manner. This results in defects in the structure or processing of collagen. The diagnosis may be confirmed with genetic testing or a skin biopsy. People may be misdiagnosed with hypochondriasis, depression, or chronic fatigue syndrome.

To date, no cure is known, however, physical therapy and bracing may help strengthen muscles and support joints. While some disorders result in a normal life expectancy, those that affect blood vessels generally result in a shorter life expectancy. EDSs affect about one in 5,000 people globally, and the prognosis depends on the specific disorder.

### EDS Classification

Hypermobile EDS (type 3 hEDS) is characterized primarily by joint hypermobility affecting both large and small joints, which may lead to recurrent joint dislocations and subluxations (partial dislocation). In general, people with this type have soft, smooth, and velvety skin with easy bruising and chronic pain of the muscles and/or bones. The mutation that causes this type of EDS is unknown. Less skin involvement is seen than other types. No genetic test for this type is available.

Classical EDS (type 1 cEDS) is associated with extremely elastic (stretchy), smooth skin that is fragile and bruises easily; wide, atrophic scars (flat or depressed scars); and joint hypermobility. Molluscoid pseudotumors (calcified hematomas over pressure points such as the elbow) and spheroids (fat-containing cysts on forearms and shins) are also frequently seen. Hypotonia and delayed motor development may occur. The mutation that causes this type of EDS is in the genes COL5A1, COL5A2, and COL1A1. It involves the skin more than hEDS.

Vascular EDS (type 4 vEDS) is characterized by thin, translucent skin that is extremely fragile and bruises easily. Arteries and certain organs such as the intestines and uterus are also fragile and prone to rupture. People with this type typically have short stature, and thin scalp hair. It also has characteristic facial features including large eyes, an undersized chin, sunken cheeks, a thin nose and lips, and ears without lobes. Joint hypermobility is present, but generally confined to the small joints (fingers, toes). Other common features include club foot, tendon and/or muscle rupture, acrogeria (premature aging of the skin of the hands and feet), early onset varicose veins, pneumothorax (collapse of a lung), recession of the gums, and a decreased amount of fat under the skin. Is can be caused by the mutations in the COL3A1 gene.

Kyphoscoliosis EDS (type 6 kEDS) is associated with severe hypotonia at birth, delayed motor development, progressive scoliosis (present from birth), and scleral fragility. Affected people may also have easy bruising, fragile arteries that are prone to rupture, unusually small corneas, and osteopenia (low bone density). Other common features include a “marfanoid habitus” which is characterized by long, slender fingers (arachnodactyly), unusually long limbs, and a sunken chest (pectus excavatum) or protruding chest (pectus carinatum). It can be caused by mutations in the gene PLOD1.

Arthrochalasia EDS (types 7A & B aEDS) is characterized by severe joint hypermobility and congenital hip dislocation. Other common features include fragile, elastic skin with easy bruising, hypotonia, kyphoscoliosis (kyphosis and scoliosis), and mild osteopenia. Type-I collagen is usually affected. It is very rare, with about 30 cases reported. It is more severe than the hypermobility type. Mutations in the genes COL1A1 and COL1A2 cause it.

Dermatosparaxis EDS (type 7C dEDS) is associated with extremely fragile skin leading to severe bruising and scarring; saggy, redundant skin, especially on the face; and hernias. It is extremely rare, with around 10 cases reported.

Brittle cornea syndrome is characterized by thin corneaa, early-onset progressive keratoglobus or keratoconus, and blue sclerae. Classic symptoms, such as hypermobile joints and hyperelastic skin, are also seen often.

Classical-like EDS (type 1 cEDS) is characterized by skin hyperextensibility with velvety skin texture and absence of atrophic scarring, generalized joint hypermobility with or without recurrent dislocations (most often shoulder and ankle), and easily bruised skin or spontaneous ecchymoses (discolorations of the skin resulting from bleeding underneath).

Spondylodysplastic EDS (spEDS) is characterized by short stature (progressive in childhood), muscle hypotonia (ranging from severe congenital, to mild later-onset), and bowing of limbs.

Musculocontractural EDS (mcEDS) is characterized by congenital multiple contractures, characteristically adduction-flexion contractures and/or talipes equinovarus (clubfoot), characteristic craniofacial features, which are evident at birth or in early infancy, and skin features such as skin hyperextensibility, bruising, skin fragility with atrophic scars, and increased palmar wrinkling.

Myopathic EDS (mEDS) is characterized by congenital muscle hypotonia and/or muscle atrophy that improves with age, proximal joint contractures (joints of the knee, hip and elbow), and hypermobility of distal joints (joints of the ankles, wrists, feet and hands).

Periodontal EDS (pEDS) is characterized by severe and intractable periodontitis of early onset (childhood or adolescence), lack of attached gingiva, pretibial plaques, and family history of a first-degree relative who meets clinical criteria.

Cardiac-valvular EDS (cvEDS) is characterized by severe progressive cardiac-valvular problems (aortic valve, mitral valve), skin problems (hyperextensibility, atrophic scars, thin skin, easy bruising), and joint hypermobility (generalized or restricted to small joints).

Methods for Treating vEDS and Related Syndromes

Included herein is a method of preventing or treating a vEDS and related syndromes in a subject in need thereof. In further embodiments, the method comprises administering to the subject an effective amount of the composition comprising the agent (e.g., For example, methods for preventing or treating vEDS and related syndromes include administering a composition comprising an agent that increases the activity or expression of a mitogen-activated protein kinase (MAPK), a protein phosphatase (PP), or a combination thereof.

In certain aspects, a small molecule compound therapeutic agent (e.g. MAPK agonist) is administered to a subject in accordance with the present methods.

In certain aspects, anisomycin or a substituted anisomycin compound or other p38 MAPK activator (MAPK agonist) is administered to a subject in accordance with the present methods, for example to prevent or treat vEDS. Exemplary substituted anisomycin compounds for administration to a subject in accordance with the present methods include for example 4-O-dodecanoyl-3-O-carba moyldeacetylanisomycin; 3-O-methylcarbamoyl-deacetylanisomycin; 4-O-acetyl-3-O-carbamoyldeacetylanisomycin; 4-O-hexanoyl-3-O-carbamoyldeacetylanisomycin; 4-O-heptanoyl-3-O-carbamoyldeacetylanisomycin; 3-O-methoxymethyldeacetylanisomycin and/or 3-O-carbamoyldeacytylanisomycin.

In certain aspects, a microcystin compound is administered to a subject in accordance with the present methods, for example to prevent or treat vEDS. In certain embodiments, the microcystin compound is selected one or more of MC-LR, MC-LA, MC-LF, MC-LW, MC-YR, and MC-RR.

In additional aspects, C2 Ceramide (also known as D-erythro-Sphingosine, N-Acetyl and may function as a PP1 activator) may be used in the present pharmaceutical compositions and administered to a subject in accordance with the present methods, for example to prevent or treat vEDS.

In additional aspects, 4-benzenesulfonyl fluoride (including a 4-benzenesulfonyl fluoride salt such as 4-benzenesulfonyl fluoride hydrochloride) may be used in the present pharmaceutical compositions and administered to a subject in accordance with the present methods, for example to prevent or treat vEDS.

MAPK agonists that may be utilized in the present methods (e.g. administered to a subject in need thereof to treat vEDS) and pharmaceutical compositions also have been disclosed e.g. in WO2010/033906 including Formulae XI, XI(a) and XI(b) thereof. In certain other aspects, however, the present therapeutic methods and compostions may not include one or more compounds disclosed WO2010/033906 including Formulae XI, XI(a) and XI(b) thereof.

Additional MPK agonists or other agents that may be used in the present therapeutic methods and pharmaceutical compositions include for example anandamide, angiotensin II, amsomycin, aurintricarboxyhc acid, 1,1-dimethylbiguamde, interlukin-11, isoproterenol, lactosyl ceramide, leukotriene D4, lipoxin A4, platelet activating factor-16, N-acetyl-D-erythro-sphingosine, N- hexanoyl-D-erythro-sphingosine, N-octanoyl-D-erythro-sphingosine, sphingosylphosphorylcholine and TNF-alpha.

Suitable agents for use in the present methods and pharmaceutical compositions (including to administer in a therapeutically effective amount to treat a vasculopathy such as vascular Ehlers-Danlos Syndrome (vEDS)) also can be determined empirically, for example in an assay (including an in vitro assay) that shows an increase of activity or expression of a mitogen-activated protein kinase (MAPK) or a protein phosphatase (PP) in the presence of a candidate compound relative to a control, for instance at least about a 5, 10, 20, 30, 40, or 50 percent or more increase in activity or expression of a mitogen-activated protein kinase (MAPK) or a protein phosphatase (PP) relative to a control. A control may be the same assay conducted without the candidate compound. See also U.S. Pat. No. 9,624,196 for assays that can be used for assessing p38 MAP kinase activity of candidate compounds.

In other embodiments, the methods for treating vEDS and related syndroms comprise administering to a subject a composition comprising an agent that increases the activity or expression of a mitogen-activated protein kinase (MAPK), a protein phosphatase (PP), or a combination thereof, produced according to the methods described herein, in combination with methods for controlling the outset of symptoms. In particular, the combination treatment can include administering readily known treatments. Additionally, combination therapy may include hormonal and/or chemotherapy (e.g. taxane-based) treatment (therapy).

The described composition can be administered as a pharmaceutically or physiologically acceptable preparation or composition containing a physiologically acceptable carrier, excipient, or diluent, and administered to the tissues of the recipient organism of interest, including humans and non-human animals.

The agent that increases the activity or expression of a mitogen-activated protein kinase (MAPK), a protein phosphatase (PP), or a combination thereof (e.g., a composition comprising the agent can be prepared by re-suspending in a suitable liquid or solution such as sterile physiological saline or other physiologically acceptable injectable aqueous liquids. The amounts of the components to be used in such compositions can be routinely determined by those having skill in the art.

In examples, for injectable administration, the composition (e.g., a composition comprising the agent that increases the activity or expression of a mitogen-activated protein kinase (MAPK), a protein phosphatase (PP), or a combination thereof is in sterile solution or suspension or can be resuspended in pharmaceutically- and physiologically-acceptable aqueous or oleaginous vehicles, which may contain preservatives, stabilizers, and material for rendering the solution or suspension isotonic with body fluids (i.e. blood) of the recipient. Non-limiting examples of excipients suitable for use include water, phosphate buffered saline, pH 7.4, 0.15 M aqueous sodium chloride solution, dextrose, glycerol, dilute ethanol, and the like, and mixtures thereof. Illustrative stabilizers are polyethylene glycol, proteins, saccharides, amino acids, inorganic acids, and organic acids, which may be used either on their own or as admixtures. The amounts or quantities, as well as the routes of administration used, are determined on an individual basis, and correspond to the amounts used in similar types of applications or indications known to those of skill in the art.

In embodiments, a therapeutically effective amount of the composition (e.g., a composition comprising an agent that increases the activity or expression of a mitogen-activated protein kinase (MAPK), a protein phosphatase (PP), or a combination thereof in humans can be any therapeutically effective amount. In one embodiment, the composition (e.g., a composition comprising the agent is administered thrice daily, twice daily, once daily, fourteen days on (four times daily, thrice daily or twice daily, or once daily) and 7 days off in a 3-week cycle, up to five or seven days on (four times daily, thrice daily or twice daily, or once daily) and 14-16 days off in 3 week cycle, or once every two days, or once a week, or once every 2 weeks, or once every 3 weeks.

In an embodiment, the composition (e.g., a composition comprising the agent is administered once a week, or once every two weeks, or once every 3 weeks or once every 4 weeks for at least 1 week, in some embodiments for 1 to 4 weeks, from 2 to 6 weeks, from 2 to 8 weeks, from 2 to 10 weeks, or from 2 to 12 weeks, 2 to 16 weeks, or longer (e.g., 1, 2, 3, 4, 5, 6, 7, 8, 9, 10, 11, 12, 36, 48, or more weeks).

Additional advantages of the methods described herein include that the agent that increases the activity or expression of a mitogen-activated protein kinase (MAPK), a protein phosphatase (PP), or a combination thereof (e.g., a composition comprising agent) can be injected systemically, as opposed to local delivery. Additional advantages include that patients requiring treatment typically require at least 1 local injections, and the injections are about 7 days apart. The compositions and methods described herein provide that patients require about 1 injection(s), systemically. In some examples, the injections can be every week.

### Pharmaceutical Compositions and Formulations

The present invention provides pharmaceutical compositions comprising an effective amount of a composition (e.g., a composition comprising an agent that increases the activity or expression of a mitogen-activated protein kinase (MAPK), a protein phosphatase (PP), or a combination thereof) and at least one pharmaceutically acceptable excipient or carrier, wherein the effective amount is as described above in connection with the methods of the invention.

In one embodiment, the composition (e.g., a composition comprising an agent that increases the activity or expression of a mitogen-activated protein kinase (MAPK), a protein phosphatase (PP), or a combination thereof) is further combined with at least one additional therapeutic agent in a single dosage form.

The term “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. Examples of pharmaceutically acceptable excipients include, without limitation, sterile liquids, water, buffered saline, ethanol, polyol (for example, glycerol, propylene glycol, liquid polyethylene glycol and the like), oils, detergents, suspending agents, carbohydrates (e.g., glucose, lactose, sucrose or dextran), antioxidants (e.g., ascorbic acid or glutathione), chelating agents, low molecular weight proteins, or suitable mixtures thereof

A pharmaceutical composition can be provided in bulk or in dosage unit form. It is especially advantageous to formulate pharmaceutical compositions in dosage unit form for ease of administration and uniformity of dosage. The term “dosage unit form” as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved. A dosage unit form can be an ampoule, a vial, a suppository, a dragee, a tablet, a capsule, an IV bag, or a single pump on an aerosol inhaler.

In therapeutic applications, the dosages vary depending on the agent, the age, weight, and clinical condition of the recipient patient, and the experience and judgment of the clinician or practitioner administering the therapy, among other factors affecting the selected dosage. Generally, the dose should be a therapeutically effective amount. Dosages can be provided in mg/kg/day units of measurement (which dose may be adjusted for the patient's weight in kg, body surface area in m², and age in years). Exemplary doses and dosages regimens for the compositions in methods of treating muscle diseases or disorders are described herein.

The pharmaceutical compositions can take any suitable form (e.g, liquids, aerosols, solutions, inhalants, mists, sprays; or solids, powders, ointments, pastes, creams, lotions, gels, patches and the like) for administration by any desired route (e.g, pulmonary, inhalation, intranasal, oral, buccal, sublingual, parenteral, subcutaneous, intravenous, intramuscular, intraperitoneal, intrapleural, intrathecal, transdermal, transmucosal, rectal, and the like). For example, a pharmaceutical composition of the invention may be in the form of an aqueous solution or powder for aerosol administration by inhalation or insufflation (either through the mouth or the nose), in the form of a tablet or capsule for oral administration; in the form of a sterile aqueous solution or dispersion suitable for administration by either direct injection or by addition to sterile infusion fluids for intravenous infusion; or in the form of a lotion, cream, foam, patch, suspension, solution, or suppository for transdermal or transmucosal administration.

In embodiments, the pharmaceutical composition comprises an injectable form.

A pharmaceutical composition can be in the form of an orally acceptable dosage form including, but not limited to, capsules, tablets, buccal forms, troches, lozenges, and oral liquids in the form of emulsions, aqueous suspensions, dispersions or solutions. Capsules may contain mixtures of a compound of the present invention with inert fillers and/or diluents such as the pharmaceutically acceptable starches (e.g., corn, potato or tapioca starch), sugars, artificial sweetening agents, powdered celluloses, such as crystalline and microcrystalline celluloses, flours, gelatins, gums, etc.

A pharmaceutical composition can be in the form of a sterile aqueous solution or dispersion suitable for parenteral administration. The term parenteral as used herein includes subcutaneous, intracutaneous, intravenous, intramuscular, intra-articular, intraarterial, intrasynovial, intrasternal, intrathecal, intralesional and intracranial injection or infusion techniques.

A pharmaceutical composition can be in the form of a sterile aqueous solution or dispersion suitable for administration by either direct injection or by addition to sterile infusion fluids for intravenous infusion, and comprises a solvent or dispersion medium containing, water, ethanol, a polyol (e.g., glycerol, propylene glycol and liquid polyethylene glycol), suitable mixtures thereof, or one or more vegetable oils. Solutions or suspensions of the compound of the present invention as a free base or pharmacologically acceptable salt can be prepared in water suitably mixed with a surfactant. Examples of suitable surfactants are given below. Dispersions can also be prepared, for example, in glycerol, liquid polyethylene glycols and mixtures of the same in oils.

The pharmaceutical compositions for use in the methods of the present invention can further comprise one or more additives in addition to any carrier or diluent (such as lactose or mannitol) that is present in the formulation. The one or more additives can comprise or consist of one or more surfactants. Surfactants typically have one or more long aliphatic chains such as fatty acids which enables them to insert directly into the lipid structures of cells to enhance drug penetration and absorption. An empirical parameter commonly used to characterize the relative hydrophilicity and hydrophobicity of surfactants is the hydrophilic-lipophilic balance (“HLB” value). Surfactants with lower HLB values are more hydrophobic, and have greater solubility in oils, while surfactants with higher HLB values are more hydrophilic, and have greater solubility in aqueous solutions. Thus, hydrophilic surfactants are generally considered to be those compounds having an HLB value greater than about 10, and hydrophobic surfactants are generally those having an HLB value less than about 10. However, these HLB values are merely a guide since for many surfactants, the HLB values can differ by as much as about 8 HLB units, depending upon the empirical method chosen to determine the HLB value. All percentages and ratios used herein, unless otherwise indicated, are by weight.

Other features and advantages of the present invention are apparent from the different examples. The provided examples illustrate different components and methodology useful in practicing the present invention. The examples do not limit the claimed invention. Based on the present disclosure the skilled artisan can identify and employ other components and methodology useful for practicing the present invention.

### Kits Comprising the Agent for Increasing the Activity or Expression of a Mitogen-Activated Protein Kinase (Mapk), a Protein Phosphatase (Pp), or a Combination Thereof

In aspects, a kit for increasing the activity or expression of MAP, PP (e.g., PP1), or p38 is provided. In embodiments, the kit comprises the agent and reagents.

In embodiments, components of the kit are suitable for delivery (e.g., local injection) to a subject.

The present invention also provides packaging and kits comprising pharmaceutical compositions for use in the methods of the present invention. The kit can comprise one or more containers selected from the group consisting of a bottle, a vial, an ampoule, a blister pack, and a syringe. The kit can further include one or more of instructions for use in treating and/or preventing a disease, condition or disorder of the present invention (e.g., a vEDS and related syndromes), one or more syringes, one or more applicators, or a sterile solution suitable for reconstituting a pharmaceutical composition of the present invention.

### EXAMPLES

The following examples illustrate certain specific embodiments of the invention and are not meant to limit the scope of the invention.

Embodiments herein are further illustrated by the following examples and detailed protocols. However, the examples are merely intended to illustrate embodiments and are not to be construed to limit the scope herein. The contents of all references and published patents and patent applications cited throughout this application are hereby incorporated by reference.

As described in the following Examples, vEDS is an inherited connective tissue disorder caused by heterozygous mutations in the COL3A1 gene, resulting in spontaneous vascular and/or organ rupture. The PLC/IP3/PKC/ERK pathway is activated in vulnerable vascular segments in a mouse model (Col3a1G939D/+) of vEDS. Attenuation of this signaling axis using either IP3/PKC or MEK/ERK inhibitors affords overt protection from vascular rupture in vEDS mice. Genetic modifiers of vascular rupture were identified in vEDS after observing that, compared to a C57BL/6J (BL6) background, vEDS mice on a 129S6/SvEvTac (129) background show near-complete life-long protection from vascular rupture and premature lethality. This protection was not associated with improved aortic wall biomechanical strength, histologic architecture, or changes in blood pressure but rather was associated with decreased activation of PKC/ERK signaling and essentially complete normalization of genome-wide mRNA expression changes as assessed by RNAseq of the descending thoracic aorta, the most vulnerable aortic segment. This unanticipated dissociation between structural tissue integrity and phenotypic outcome provided the first evidence that a nonproductive cellular response to an altered extracellular matrix, rather than the matrix deficiency per se, is the dominant determinant of vascular disease in vEDS, boding well for the identification of pharmacologic interventions.

Genome-wide genotyping of intercrossed BL6/129 vEDS mice stratified by survival identified a single significant protective locus on mouse chromosome 11 (OR=0.2412, p=3.73E-5)M. ap2k6, encoding a p38-activating kinase, emerged as the only likely candidate gene based on both expression in the aorta and predicted functional strain-specific sequence variation (p. G76E). Protected 129 vEDS mice showed higher expression of Map2k6 in the aorta and increased phosphorylation of p38. Increased activation of p38 associated with increased activity of its substrate PP1 in the aortic wall, a phosphatase that dephosphorylates and deactivates pPKC and pERK. Inhibition of this protective axis using a selective p38 inhibitor significantly accelerated vascular rupture in vEDS mice in a PKC and ERK-dependent manner. These results both validate and extend the understanding of cellular signaling events that culminate in vascular rupture in vEDS and define a pathway of natural potent protective disease modification. Pharmacologic interventions that mimic nature's successful modification strategy will afford substantial protection to patients with vEDS.

### Example 1: 129 Background Protects Col3a1^{G938D/+} Mice from Aortic Rupture

Heterozygous mice were characterized for a disease-causing Col3a1 allele (Col3a1/^{G938D/+}) on a pure BL6 mouse background. These mice recapitulate severe vEDS phenotypes, with spontaneous death due to aortic rupture, leading to a median survival of only 45 days(13). The BL6 vEDS mice were backcrossed onto a 129 background to assess the impact of mouse strain on disease severity.

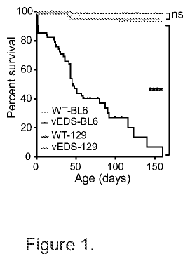

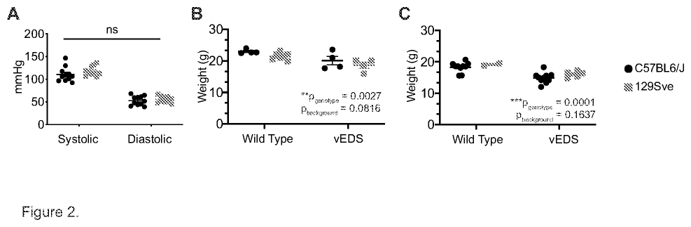

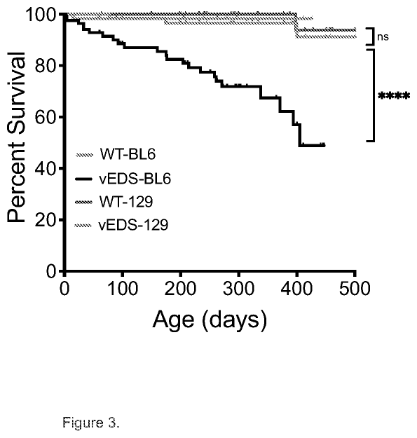

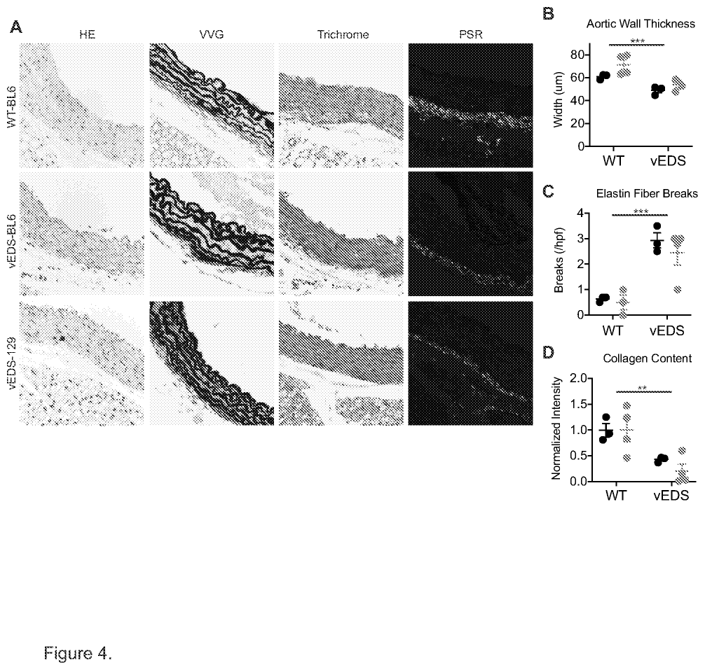

Remarkably, the 129 background led to essentially complete protection from aortic rupture and premature death, with the long-term performance of these mice indistinguishable from that observed for wild-type littermates (93% vs 98% survival after 6 months) (FIG. 1). This was independent of hemodynamic status, weight, and gender (FIGS. 2A-2C), although both BL6 and 129 vEDS mice are smaller than their wild-type littermates. The same protective effect of the 129 background was also observed in a less severe vEDS mouse model (Col3a1^{G209S/+}) (FIG. 3). Although aortic wall architecture is relatively preserved in BL6 vEDS mice, minor alterations included occasional elastic fiber breaks, decreased aortic wall thickness, and decreased collagen content at 2 months of age (13). Each of these aortic wall abnormalities was maintained in 129 vEDS aortas, despite their improved survival (FIGS. 4A-4D).

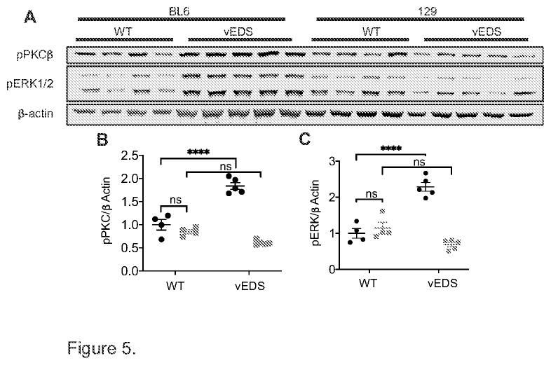

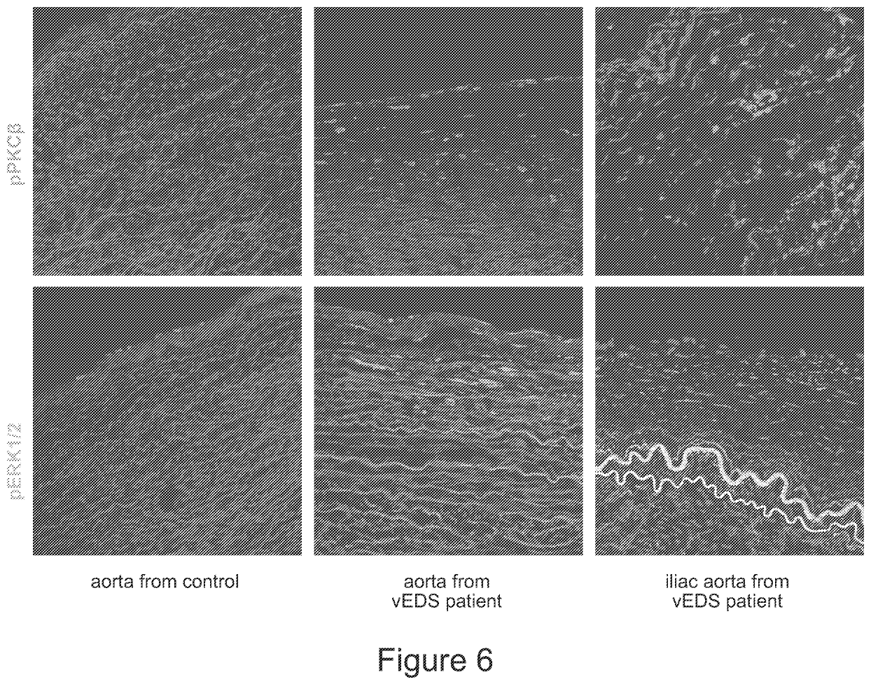

Given that the PLC/IP3/PKC/ERK axis was implicated in vEDS aortic pathology, the status of PKC and ERK activation was investigated in the 129 vEDS aortas by western blot analysis. Activation of ERK1/2 and PKC was accentuated in BL6 vEDS aortas, while the level of activation in 129 vEDS aortas was not different from wildtype littermate aortas (FIGS. 5A-5C). There was no difference in activation status of these signaling molecules in wild type animals on the two backgrounds. Moreover, vascular tissue from humans with vEDS also demonstrated increased levels of PKC and ERK activation, closely resembling the susceptible BL6 vEDS mouse aortas (FIG. 6). This indicated that the method by which 129 vEDS aortas are able to prevent vascular rupture had direct relevance to the human condition.

### Example 2: Identification of a Genetic Modifier Using Mixed Background Mice

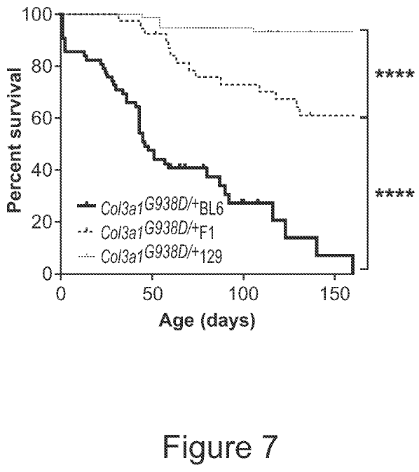

To identify variants in genes that modify vascular rupture risk in vEDS, animals were stratified based on survival. Extremes of this distribution were investigated to maximize both signal intensity and power, using the Col3a1^{G938D/+} model which shows early death due to vascular rupture. Greater than 63% of all vEDS mice on the BL6 background die by 50 days of age, while >90% of vEDS mice on the 129 background live longer than 6 months. Due to ambiguity regarding the cause of perinatal lethality and the associated difficulty in reliable sample collection, a “severe phenotype” was defined as mice that died from aortic rupture between 1 and 7 weeks and defined a “mild phenotype” as mice that showed survival past 24 weeks of age. vEDS mice that have been backcrossed one time (F1 generation) showed improved long-term survival similar to completely backcrossed 129 vEDS mice, suggesting the presence of a genetic modifier that acts in an autosomal dominant manner (FIG. 7).

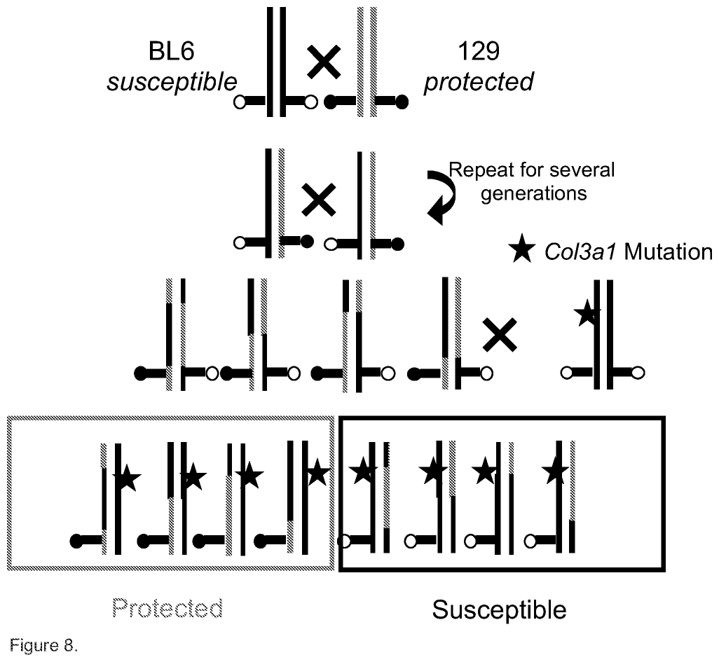

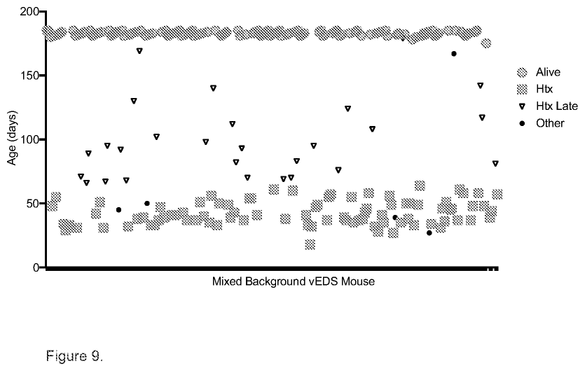

Wild-type BL6 and 129 animals were intercrossed, and subsequently their progeny for 4 generations, to introduce extensive recombination between the strain-specific chromosomes, with expansion of numbers of mice in each generation. These mixed genetic mice were bred to BL6 vEDS mice to produce a large population with a mixed but mostly BL6 genetic background, from which an autosomal dominant protective allele was identified (FIG. 8). This mixed-background population stratified into “mild” and “severe” phenotypic groups as expected (FIG. 9). Male vEDS mice were more likely than female vEDS mice to be categorized as “severe” (p<0.0001).

91 mixed background vEDS mice were genotyped with a mild phenotype, and 96 mixed background vEDS mice with a severe phenotype. Sufficient animals of both sexes were included for use in combined and parallel sex-specific analyses, due to evidence in humans that males with vEDS have a more severe vascular phenotype, as well the possibility of sex-specific modification mechanisms (8,13). A linkage disequilibrium (LD) block-pruned set of 603 SNPs was used to perform logistic regression GWAS, assuming full dominance for the minor allele and using sex as a covariate.

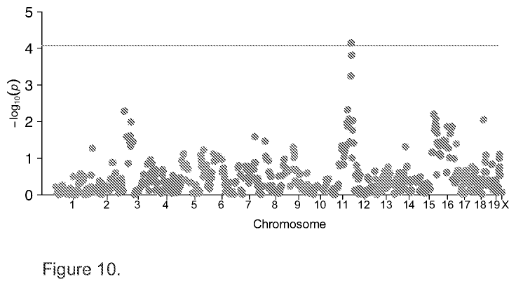

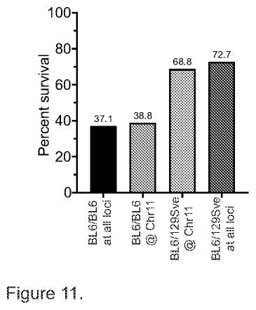

1 locus was identified that was linked with protection from aortic rupture on chromosome 11, with a peak p-value of 7.081E-03 (FIG. 10). The odds ratio at the locus is 0.22293, indicating that the presence of one 129 allele at the locus decreases the odds of early death due to aortic rupture (FIG. 11).

Surprisingly, despite the sex specific effects in the incidence of aortic rupture, no association signal on the chromosome X was observed. This suggested that the increased severity in males is not attributable to an X-linked trait but rather an autosomal trait that is sex-limited (i.e. androgen production). Male and female mice were separated and GWAS logistic analysis was performed in each cohort, hypothesizing that there were sex-specific autosomal modifiers of disease. Unfortunately, when split by sex, the cohorts individually are under-powered, and no suggestive signals are revealed.

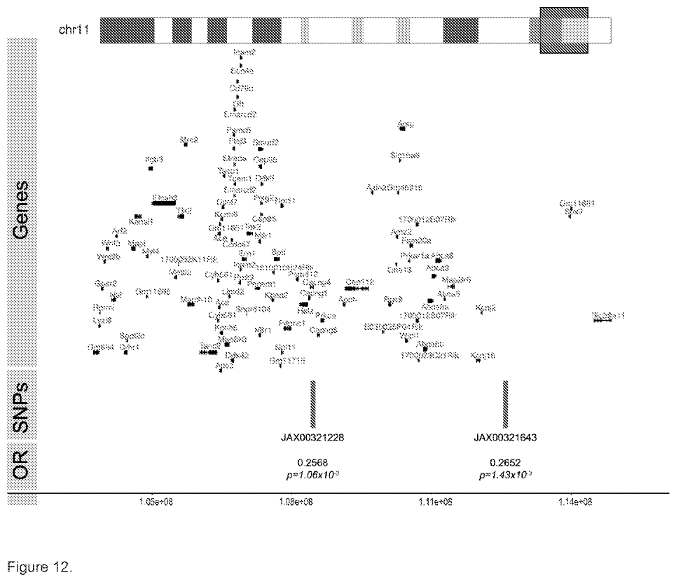

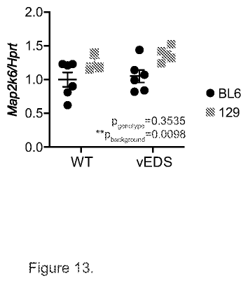

A candidate functional variant within the locus was investigated that differed between the backgrounds. The area of formal analysis was a 10.1 Mb region, including the region between the closest neighboring upstream and downstream SNP markers below the peak on chromosome 11 (103,513,550-113,599,747 bp) (FIG. 12). Within this region, genes were filtered that were expressed in the aorta that had functional differences between BL6 and 129-defined as splice variants, predicted functional missense variants, or expression differences between the two backgrounds. 9 genes in this locus were differentially expressed between BL6 and 129, as determined by RNA transcriptome sequencing of wild type BL6 and 129 proximal descending aortas. 5 genes at this locus contained a variant that was predicted to have a functional effect (Table 1). The missense variant in Map2k6 (rs51129320; n.11:110490856-110490856G>A; c.G227A; p.G76E; PROVEAN score −3.66) became the top candidate variant. Map2k6, which encodes for Map kinase kinase-6 (MKK6), is a p38-activating kinase known to affect MAPK/ERK signaling. Map2k6 was expressed at a higher level in 129 mice compared to BL6 (FIG. 13). Interestingly, this exact variant had previously been identified to modify Marfan syndrome phenotypes in mouse models, albeit in the opposite direction(10). This may relate to a different location for aneurysm in Marfan syndrome (the aortic root) compared to vEDS (the descending aorta or peripheral arteries). It may also relate to a different repertoire of growth factors that are implicated in Marfan syndrome, prominently including transforming growth factor β (TGFβ).

### Example 3: Map2k6 Affects Vascular Rupture Risk in vEDS

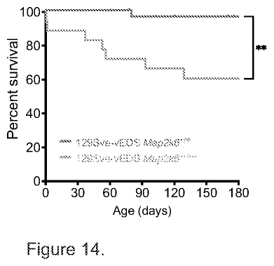

To assess the role of Map2k6 on vascular rupture risk in vEDS, BL6 and 129 background vEDS mice were crossed to Map2k6^{-/-} mice to generate vEDS mice haploinsufficient or fully deficient for Map2k6. On a BL6 background, zero vEDS mice survived past P1 with one copy of Map2k6 deleted (Col3a1^{G938D/+}Map2k6^{+/-}), suggesting that the loss of one Map2k6 allele in BL6 vEDS mice was significantly lethal in the prenatal or early postnatal period (Table 2, below). 129 background vEDS mice with a complete loss of Map2k6 have an elevated risk of vascular rupture (FIG. 14). These data established that the function of Map2k6 is critical for survival in vEDS.

TABLE 2
Loss of one copy of Map2k6 results in early death
in BL6 vEDS mice. Significant differences were determined
using Fishers Exact Test (p = 0.0045).

| | vEDS | WT |
|---|---|---|
| Observed | | |
| M2K6 +/+ | 5 | 34 |
| M2K6 +/− | 0 | 18 |
| Expected | | |
| M2K6 +/+ | 8 | 21 |
| M2K6 +/− | 8 | 21 |

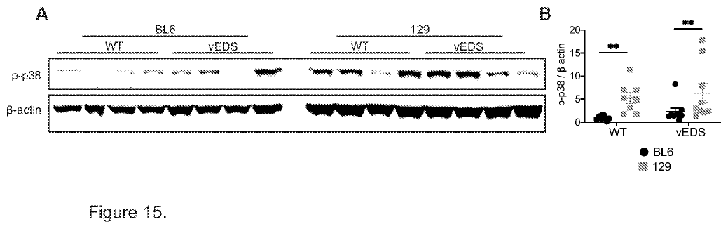

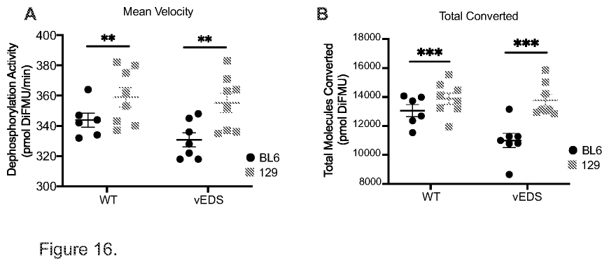

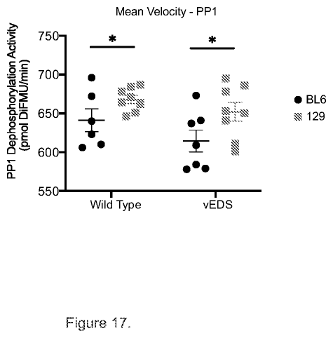

Previous work described that the Map2k6 BL6 allele leads to decreased function compared to the 129 allele (10). MKK6, together with MAP3K4, is known to increase p38 activation, which in turn negatively regulates PKC and ERK activation via protein phosphatase 1 and 2A (PP1/2A) (14-16). Within the 129 background, the increased MKK6 activity lead to increased p38 activation and hence increased PP1/2A activity, with consequent reduction in PKC and ERK activation (FIGS. 4A-4D). Indeed, immunoblots showed that enhanced p38 activation was seen in 129 background mice (FIG. 15A). Furthermore, protein phosphatase activity was increased in 129 background mice compared to BL6 background mice (FIGS. 16A-16B). The observed difference in phosphatase activity was mostly driven by differences in PP1 activity (FIG. 17).

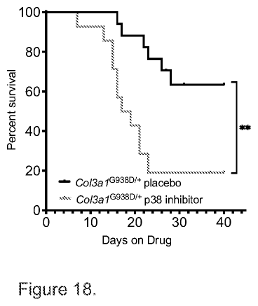

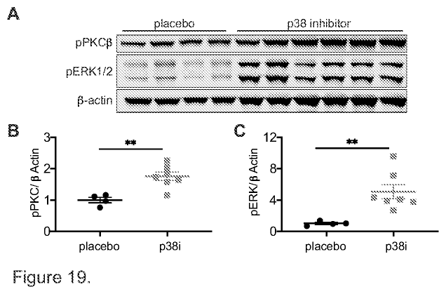

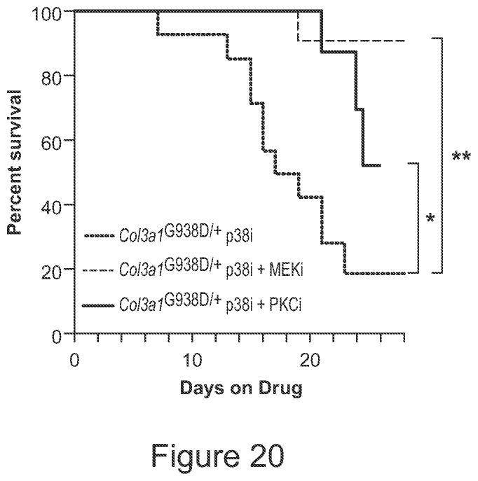

The hypothesis that the elevated p38 and PP1 activity led to protection from vascular rupture in vEDS was investigated. To examine the role of elevated p38 activity on vascular rupture directly, BL6 vEDS mice were treated with SB203580, a selective p38 inhibitor. Treatment with the p38 inhibitor significantly increased the rate of death in BL6 vEDS mice (FIG. 18), suggesting that p38 activity has a significant protective effect on the risk of vascular rupture in vEDS mice. The elevated risk of rupture in BL6 vEDS mice treated with a p38 inhibitor correlated with the expected increase in PKC and ERK activation (FIGS. 19A-19C). The elevated risk of rupture was also rescued by concomitant treatment with either a MEK inhibitor (cobimetinib) or a PKC inhibitor (ruboxistaurin), demonstrating that p38 prevents vascular rupture in a PKC/ERK dependent manner (FIG. 20). These data suggest that p38 activity and PP1 activity, both of which are elevated in 129 background vEDS mice, offer significant protection from vascular rupture in susceptible vEDS mice.

### DISCUSSION

Provided herein, it was demonstrated that the 129 background attenuates the PLC/PKC/ERK signaling cascade in association with prevention of vascular events and premature death in vEDS mice. Remarkably, this rescue on the 129 background is not dependent upon an improvement in collagen deposition or inherent structural integrity of the aortic wall. Instead, phenotypic rescue appears to be dependent on the biochemical response of vascular cells. This bodes well for the development of pharmacologic strategies that can mimic this mechanism of protection. As such, p38 or PP1 activation may represent novel therapeutic strategies for reducing rupture risk in vEDS. This confluence of discovery-based and hypothesis-driven interrogations informs disease pathogenesis of vEDS and provides added confidence regarding the potential of therapeutic strategies targeting the PKC/ERK1/2 axis of activation.

GWAS was used in inbred mouse strains with differing disease severity and rigorous follow up biochemical and functional analyses to identify and validate a genetic modifier that protects vEDS mice from vascular rupture. This association directly implicated a region on chromosome 11 which contained only one functional variant between the two strains of interest. Map2k6 expression was higher in 129 background mice and it contained a variant which led to elevated MKK6 activity. Correlating with improved survival, increased p38 phosphorylation and elevated PP1/2A activity was observed on the 129 background, suggesting that activation of this pathway was protective in vEDS.

A critical test of hypothesis was to show that directed provocations altered the survival of the vEDS mice in a predictable and robust manner. vEDS mice were deficient in either Map2k6 or p38 activity significantly worsened survival. Together these data indicated that Map2k6 was responsible, at least in part, for the protective effect that the 129 background has on vEDS vascular phenotypes. This work defines MKK6/p38 as a negative regulator of the altered molecular signaling axis that is responsible for the increase in vascular rupture risk in vEDS mice.

The chromosome 11 locus, as defined by the 10.1 Mb interval between the neighboring SNPs to the two genome-wide significant SNPs, contained 117 genes. The Map2k6 variant was based on the hypothesis that the modifier would lead to a functional protein difference in a gene expressed in the aorta, however this is not enough to definitively exclude other genes at this locus. Importantly, all of the experimental results confirmed that Map2k6 played a protective role in vascular rupture risk, as deletion of Map2k6 or inhibition of its direct downstream target, p38, significantly increased the risk of early death and vascular rupture. While these data support a role for MKK6 and p38 in vascular rupture risk, they do not exclude any of the other genes at this locus. Furthermore, this variant alone does not explain the full protective effect of the 129 background on vEDS rupture risk, suggesting that there are other relevant variants that remain to be identified.

The work herein provides that p38 is working to negatively regulate the PKC/ERK axis via induction of PP1 activity. p38 has also previously been implicated in the negative regulation of androgen receptor activity (17,18). Exploration of the effects of the Map2k6 variant on androgen receptor signaling will be important for understanding the sexual dimorphism identified in this study.

While the genetic background affects vEDS phenotypes in an opposite manner as MFS phenotypes, robust unbiased whole genome association analysis showed genome-wide significance for the same locus and putative single gene variation. MFS aortic disease exclusively affects the aortic root, while vEDS vascular disease can occur in any medium or large arterial segment, generally excluding the aortic root. Furthermore, p38 has been demonstrated to potentiate SMAD signaling, which plays a critical role in MFS but not in vEDS (19-21).

Methods

Mice

Mice were maintained either on a C57BL/6J background (#000664, The Jackson Laboratory) or on a 129S6/SvEvTac background (#129SVE, Taconic Biosciences). Mice were considered to be 129S6/SvEvTac after backcrossing for a minimum of 4 generations. F1 and mixed mice were generated by interbreeding pure C57BL/6J and 129S6/SvEvTac mice. To identify the dominant genetic locus, mixed background wild-type females were bred to Col3a1^{G938D/+} male mice on a pure BL6 background. Mice haploinsufficient or knockout for Map2k6 were originally obtained from the Jackson Laboratory (#008382) and backcrossed in the lab for a minimum of 10 generations. Restriction enzymes were used to detect the presence or absence of the Col3a1 mutation. The G209S mutation leads to the loss of an AvaII cut site and the G938D mutation leads to the gain of a BamHI cut site (AvaII R0153L; BamHI-HF R3136S, New England Biolabs). All mice found dead were assessed for cause of death by necropsy, noting in particular hemothorax and hemoperitoneum.

Histology and Immunofluorescence

Mice were euthanized by isoflurane inhalation and the left common iliac artery was transected to allow for drainage. PBS and PBS containing 4% paraformaldehyde (PFA) was flushed through the left ventricle. The heart and thoracic aorta were removed en block and fixed in 4% PFA overnight at 4° C. Aortas were submitted for paraffin fixation and longitudinal sections 5 micrometers thick were mounted on glass slides and stained with hematoxylin & eosin (HE), Verhoeff-van Giesen (VVG), Masson's Trichrome, or Picrosirius red (PSR). Slides were imaged at 20× and 40× magnification using a Nikon Eclipse E400 microscope. Collagen content was determined by polarized PSR intensity (22) and elastin breaks were counted by a researcher blinded to genotype and treatment arm using only VVG-stained sections where elastin breaks were clearly visualized.

Human samples were obtained from surgical pathology records. Tissue samples were submitted for paraffin fixation after fixation in formaldehyde. Longitudinal sections 5 micrometers thick were mounted on glass slides. Slides were deparaffinized and incubated in antigen retrieval solution in a pressure cooker for 1 minute (Vector labs, H3300). After this antigen retrieval step, sections were incubated with 1% BSA for 1 hour at room temperature. Primary antibodies were diluted at 1:200 in blocking buffer (1% BSA) and incubated overnight at 4° C. Three consecutive washes were performed prior to incubation with anti-rabbit secondary antibody conjugated to Alexa Fluor 594 (Invitrogen, R37119) or Alexa Fluor 488 (Invitrogen, A21206) at 1:100 for 1 hour. Slides were again washed 3 times, incubated with DAPI nuclear stain at 1:40,000 for 10 minutes, and washed again prior to mounting with VECTASHIELD Hard Set Mounting Media (H-1400). The following primary antibodies were used: anti-phospho ERK1/2 (Cell Signaling Technology, 4370), anti-PKCβ (phospho 5660) (Abcam, 75837). Images were acquired on a Zeiss LSM780-FCS confocal microscope at ×20 magnification and are presented as maximal intensity projection. Image adjustments to enhance visualization of information present in the original were applied equally across samples.

### Western Blot

Descending thoracic aortas (distal to the left subclavian branch and proximal to the diaphragm) from mice that did not die from aortic rupture and did not have any overt pathology at the time of planned sacrifice were harvested, snap frozen in liquid nitrogen, and stored at −80° C. until processed. Protein was extracted using an automatic bead homogenizer in conjunction with a Protein Extraction Kit (Full Moon Biosystems). All protein lysis buffers contained both PhosSTOP and Complete™, Mini, EDTA-free Protease Inhibitor Cocktail (Roche). Western blotting was performed using LI-COR buffer and species appropriate secondary antibodies conjugated to IR-dye700 or IRdye-800 (LI-COR Biosciences), according to the manufacturer's guidelines and analyzed using LI-COR Odyssey. The following primary antibodies were used: anti-β-Actin (8H10D10) (Cell Signaling Technology, 3700), anti-phospho ERK1/2 (Cell Signaling Technology, 4370), anti-PKCβ (phospho 5660) (Abcam, 75837), anti-phospho p38 (Cell Signaling Technology, 4511).

### RNAseq

RNA was isolated from the proximal descending thoracic aorta of three mice for each condition, flushed in PBS, and directly stored into TRIzol (Invitrogen). RNA was extracted according to manufacturer's instructions and purified using the PureLink RNA Mini Kit (Invitrogen). Library prep was performed using TruSeq Stranded Total RNA with Ribo-Zero (Illumina). Sequencing was run on an Illumina HiSeq2500 using standard protocols.

Illumina's CASAVA 1.8.4 was used to convert BCL files to FASTQ files. Default parameters were used. rsem-1.3.0 was used for running the alignments as well as generating gene and transcript expression levels. The data was aligned to “mm10” reference genome. EBseq was used for Differential Expression analysis and default parameters were used(25).

### Gene Expression

Aortas were dissected as described above, flushed in PBS, and directly stored in TRIzol (Invitrogen). RNA was extracted according to the manufacturer's instructions and purified with RNAeasy mini columns (Qiagen). Complementary DNA (cDNA) was generated using TaqMan High Capacity cDNA Reverse Transcription reagents (Applied Biosystems) and qPCR was performed in triplicate with TaqMan Universal PCR Master Mix (Applied Biosystems). The following TaqMan probes were used: Mm00803694 ml (Map2k6), Mm00446968 ml (Hprt). Relative quantification for each transcript was obtained by normalizing against Hprt transcript abundance according to the formula 2^{(-Ct)}/2^{(-Ct Hprf)}. All expression levels were normalized to untreated wild-type control expression levels.

### Delivery of Medication

For drug trials in the Col3a1/^{G938D/+} mice, mice were initiated on medication at weaning and continued until the end of the trial. Cobimetinib (GDC-0973/R0551404, Active Biochem) was dissolved in drinking water and filtered to reach a final concentration of 0.02 g/L giving an estimated dose of 2 mg/kg/day. Ruboxistaurin (LY333531 HCl, Selleck Chemicals) was mixed with powdered food (LabDiet) to give a concentration of 0.1 mg/g giving an estimated dose of 8 mg/kg/day. SB203580 (Selleck Chemicals) was administered at a dose of 5 mg/kg/d every 3 days by intraperitoneal injection. Animals receiving placebo (5% Tween in PBS) were also injected every 3 days by intraperitoneal injection.

### Blood Pressure Analysis

Blood pressures were measured by tail cuff plethysmography one week prior to completion of a study. To measure and record blood pressures, the BP-2000 Blood Pressure Analysis system was utilized. This method utilizes variations in the amount of light transmitted through the tail as the basic signal that is analyzed to determine the blood pressure and pulse rate. After the software determines the pulse rate, it inflates the occlusion cuff and records diastolic pressure when the wave form starts to decrease and systolic pressure when the waveform remains at a steady value. If either measurement is unclear to the software, it is not recorded. Mice were habituated to the system for three days prior to collection in which 10-15 measurements were obtained and averaged.

### Mouse Whole Genome Analysis

Genotyping was performed using the GigaMUGA array, which has been optimized to interrogate SNPs that allow discrimination of very closely related mouse strains, such as C57BL6/J and 129SveTac. Standard GoldenGate chemistry was applied on an iScan mircroarray scanner. SNPs were called using GenomeStudio version 2011.1, Genotyping Module version 1.9.4, and GenTrain Version 1.0 with Genome Build 37. A total of 143,446 SNPs were attempted per individual (137,746 autosomal, 5,601 X chromosome, and 99 Y chromosome SNPs).

SNPs with less than 90% call rate were filtered as assay failures, which eliminated 3,942. SNPs were filtered as uninformative using the following criteria: if the Minor Allele Frequency (MAF) equaled 0 (removing 97,595 SNPs), if the heterozygote rate (AB Freq) was greater than 80% or equal to 0 (removing an additional 707 SNPs), or if the homozygote rate (AA Freq or BB Freq) was equal to 0 (removing an additional 6,461 SNPs). Genotype frequencies were investigated for parental strains (B6, 129) and F1 samples (50/50 mix of B6 and 129) and any SNP that did not have the 2 parental strains as opposite homozygotes was filtered as uninformative (AA or BB Freq=0) and any SNP that did not have F1 samples as heterozygotes was filtered as uninformative (AB Freq=0). In total, 32,218 SNPs were released per individual. Of the 5,573,714 released SNPs, only 7,917 no calls were made, indicating a missing data rate of 0.14%. PLINK software was utilized to prune SNPs to include only one per LD block using default values. PLINK software was used to calculate genome wide association using logistic analysis, covariate for sex, and an autosomal dominant allele assumption.

### Phosphatase Assay

Descending thoracic aortas (distal to the left subclavian branch and proximal to the diaphragm) from mice that did not die from aortic rupture and did not have any overt pathology at the time of planned sacrifice (at 2 months of age for all samples unless otherwise stated) were harvested, snap frozen in liquid nitrogen, and stored at −80° C. until processed. Protein was extracted using an automatic bead homogenizer in conjunction with a Protein Extraction Kit (Full Moon Biosystems). All protein lysis buffers for downstream phosphatase assay analysis contained Complete™, Mini, EDTA-free Protease Inhibitor Cocktail (Roche). Lysed protein was subjected to a direct fluorescence-based assay for detecting serine/threonine phosphatase activity (RediPlate™ 96 EnzChek serine/threonine phosphatases Assay Kit, Molecular Probes) according to the manufacturer's instructions. Briefly, appropriate buffers for either the serine/threonine phosphatases PP1 and PP2A were added to a 96-well microplate preloaded with inhibitors of phosphatases other than serine/threonine phosphatases, and with the fluorogenic serine/threonine phosphate substrate DiFMUP (6,8-difluoro-4-methyl-umbelliferyl phosphate), from which DiFMU is generated. The fluorescence emitted by converted DiFMU was measured using a fluorescence microplate reader, with excitation at 355±20 nm and emission at 460±12.5 nm.

### Statistics

All data points are presented for quantitative data, with an overlay of the mean with SEM. All statistical analysis was performed using GraphPad Prism 8 unless otherwise noted. For data that did not pass Shapiro-Wilk normality tests, Kruskal-Wallis (nonparametric) testswere performed to evaluate significance between groups using Dunn's multiple comparison test with a p-value of <0.05 considered statistically significant. For data that did pass normality, two-way or one-way ANOVA was used with multiple comparisons, as noted in each legend.

For single comparisons, if the Shapiro-Wilk normality test was passed, then two-tailed unpaired t-tests were performed. If Shapiro-Wilk normality test did not pass, then Mann-Whitney nonparametric tests were performed.

Kaplan-Meier survival curves were compared using a log-rank (Mantel-Cox) test. Mice were censored only if unrelated to the outcome, such as for planned biochemical or histologic analysis or if the authors were directed to euthanize them by animal care staff, for malocclusion, fight wounds, or genital prolapse.

### REFERENCES

1. Shalhub S, Byers P H, Hicks K L, et al. A multi-institutional experience in the aortic and arterial pathology in individuals with genetically confirmed vascular Ehlers-Danlos syndrome. J Vasc Surg. May 2019. doi:10.1016/J.JVS.2019.01.069
2. Pepin M, Schwarze U, Superti-Furga A, Byers PH. Clinical and Genetic Features of Ehlers-Danlos Syndrome Type IV, the Vascular Type. N Engl J Med. 2000; 342(342):673-680. doi:10.1056/NEJM200003093421001
3. Pepin M G, Schwarze U, Rice K M, Liu M, Leistritz Dru, Byers PH. Survival is affected by mutation type and molecular mechanism in vascular Ehlers-Danlos syndrome (EDS type IV). Genet Med. 2014; 16(12):881-888. doi:10.1038/gim.2014.72
4. Smith L B, Hadoke PWF, Dyer E, et al. Haploinsufficiency of the murine Col3a1 locus causes aortic dissection: A novel model of the vascular type of EhlersDanlos syndrome. Cardiovasc Res. 2011; 90(1):182-190. doi:10.1093/cvr/cvq356
5. Smith L T, Schwarze U, Goldstein J, Byers PH. Mutations in the COL3A1 gene result in the Ehlers-Danlos syndrome type IV and alterations in the size and distribution of the major collagen fibrils of the dermis. J Invest Dermatol. 1997; 108(3):241-247. doi:10.1111/1523-1747.ep12286441
6. Leistritz D F, Pepin M G, Schwarze U, Byers PH. COL3A1 haploinsufficiency results in a variety of Ehlers-Danlos syndrome type IV with delayed onset of complications and longer life expectancy. Genet Med. 2011; 13(8):717-722. doi:10.1097/GIM.0b013e3182180c89
7. Cortini F, Marinelli B, Romi S, et al. A New COL3A1 Mutation in Ehlers-Danlos Syndrome Vascular Type With Different Phenotypes in the Same Family. Vasc Endovascular Surg. 2017; 51(3):141-145. doi:10.1177/1538574417692114
8. Byers P H, Belmont J, Black J, et al. Diagnosis, natural history, and management in vascular Ehlers-Danlos syndrome; Diagnosis, natural history, and management in vascular Ehlers-Danlos syndrome. Am J Med Genet Part C (Seminars Med Genet. 2017; 175:40-47. doi:10.1002/ajmg.c.31553
9. Kuivaniemi H, Tromp G. Type III collagen (COL3A1): Gene and protein structure, tissue distribution, and associated diseases. Gene. 2019; 707:151-171. doi:10.1016/j.gene.2019.05.003
10. Doyle J J, Doyle A J, Wardlow R, et al. Identification of Major Genetic Modifiers of Aortic Aneursym in Marfan Syndrome. Baltimore, MD; 2020.
11. Lima B L, Santos EJC, Fernandes G R, et al. A new mouse model for marfan syndrome presents phenotypic variability associated with the genetic background and overall levels of Fbn1 expression. PLoS One. 2010; 5(11):e14136. doi:10.1371/journal.pone.0014136
12. Fernandes G R, Massironi SMG, Pereira L V. Identification of Loci Modulating the Cardiovascular and Skeletal Phenotypes of Marfan Syndrome in Mice. Sci Rep. 2016; 6:22426. doi:10.1038/5rep22426
13. Bowen C J, Giadrosic JFC, Burger Z, et al. Targetable cellular signaling events mediate vascular pathology in vascular Ehlers-Danlos syndrome. J Clin Invest. 2020; 130(2):686-698. doi:10.1172/JC1130730
14. Zarubin T, Han J. Activation and signaling of the p38 MAP kinase pathway. Cell Res. 2005; 15(1):11-18. doi:10.1038/sj.cr.7290257
15. Han J, Lee J D, Jiang Y, Li Z, Feng L, Ulevitch R J. Characterization of the structure and function of a novel MAP kinase kinase (MKK6). J Biol Chem. 1996; 271(6):2886-2891. doi:10.1074/jbc.271.6.2886
16. Keranen L M, Dutil E M, Newton A C. Protein kinase C is regulated in vivo by three functionally distinct phosphorylations. Curr Biol. 1995; 5(12):1394-1403. doi:10.1016/50960-9822(95)00277-6
17. Gioeli D, Black B E, Gordon V, et al. Stress Kinase Signaling Regulates Androgen Receptor Phosphorylation, Transcription, and Localization. Mol Endocrinol. 2006; 20(3):503-515. doi:10.1210/me.2005-0351
18. Koryakina Y, Ta H Q, Gioeli D. Androgen receptor phosphorylation: Biological context and functional consequences. Endocr Relat Cancer. 2014;21(4):T131. doi:10.1530/ERC-13-0472
19. Carta L, Smaldone S, Zilberberg L, et al. p38 MAPK is an early determinant of promiscuous Smad2/3 signaling in the aortas of fibrillin-1 (Fbn1)-null mice. J Biol Chem. 2009; 284(9):5630-5636. doi:10.1074/jbc.M806962200
20. Wrighton K H, Lin X, Feng X H. Phospho-control of TGF-β superfamily signaling. Cell Res. 2009; 19(1):8-20. doi:10.1038/cr.2008.327
21. Hayes S A, Huang X, Kambhampati S, Platanias L C, Bergan R C. p38 MAP kinase modulates Smad-dependent changes in human prostate cell adhesion. Oncogene. 2003; 22(31):4841-4850. doi:10.1038/sj.onc.1206730
22. Vogel B, Siebert H, Hofmann U, Frantz S. Determination of collagen content within picrosirius red stained paraffin-embedded tissue sections using fluorescence microscopy. MethodsX 2015; 2:124-134. doi:10.1016/J.MEX.2015.02.007
23. Daugherty A, Rateri D L, Charo I F, Owens A P, Howatt D A, Cassis L A. Angiotensin II infusion promotes ascending aortic aneurysms: attenuation by CCR2 deficiency in apoE ^{-/-} mice. Clin Sci. 2010; 118(11):681-689. doi:10.1042/CS20090372
24. Isaacs M N, Anidjar S N, Dobrin P B, Eichorst M B, Graham G P. Correlation of inflammatory infiltrate with the enlargement of experimental aortic aneurysms. J Vasc Surg. 1992;16(2):a35585. doi:10.1067/mva.1992.35585
25. Leng N, Dawson J A, Thomson J A, et al. EBSeq: an empirical Bayes hierarchical model for inference in RNA-seq experiments. Bioinformatics. 2013; 29(8):1035-1043. doi:10.1093/bioinformatics/btt087

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All references, e.g. ,U U.S. patents, U.S. patent application publications, PCT patent applications designating the U.S., published foreign patents and patent applications cited herein are incorporated herein by reference in their entireties. Genbank and NCBI submissions indicated by accession number cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

While this invention has been particularly shown and described with references to preferred embodiments thereof, it will be understood by those skilled in the art that various changes in form and details may be made therein without departing from the scope of the invention encompassed by the appended claims.

1. A method of treating or preventing a vasculopathy in a subject, the method comprising:
administering an effective amount of an agent, wherein the agent increases the activity or expression of a mitogen-activated protein kinase (MAPK), a protein phosphatase (PP), or a combination thereof, and
thereby treating the vasculopathy.

2. The method of claim 1, wherein the vasculopathy comprises vascular Ehlers-Danlos Syndrome (vEDS).

3. The method of claim 1, wherein the agent comprises an antibody or fragment thereof, a polypeptide, a small molecule, a nucleic acid molecule, or any combination thereof.

4. The method of claim 1, wherein the MAPK comprises p38.

5. The method of claim 4, wherein the p38 comprises p38-α, p38-β, p38-γ, or p38-δ.

6. The method of claim 1, further comprising administering an agent that decreases the activity or expression of extracellular signal-regulated kinase (ERK), protein kinase C (PKC).

7. The method of claim 1, wherein the protein phosphatase comprises protein phosphatase 1 (PP1), protein phosphatase 2A (PP2A), or a combination thereof.

8. The method of claim 1, wherein the agent comprises a small molecule.

9. The method of claim 1, wherein the effective amount of the agent is from about 0.001 mg/kg to 250 mg/kg body weight.

10. The method of claim 1, wherein the subject comprises a level of MAPK or PP protein or mRNA that is different than a normal control.

11. The method of claim 10, wherein the subject comprises a level of E MAPK or PP protein or mRNA that is at least about 10% higher compared to a normal control.

12. The method of claim 10, wherein the subject comprises a level of MAPK or PP activity that is at least about 10% higher compared to a normal control.

13. The method of claim 10, wherein the level is in a test sample obtained from the subject.

14. The method of claim 13, wherein the test sample comprises blood, serum, plasma, saliva, tears, vitreous, cerebrospinal fluid, sweat, cerebrospinal fluid, or urine.

15. A method of treating or preventing a vasculopathy in a subject, the method comprising:
administering an effective amount of an agent, wherein the agent increases the activity of mitogen-activated protein kinase (MAPK) pathway signaling, wherein the MAPK comprises Map2k6, and
thereby treating the vasculopathy.

16. The method of claim 15, wherein the vasculopathy comprises vascular Ehlers-Danlos Syndrome (vEDS).

17. The method of claim 15, wherein the agent comprises an antibody or fragment thereof, a polypeptide, a small molecule, a nucleic acid molecule, or any combination thereof.

18. A pharmaceutical composition for the treatment of a vasculopathy, the composition comprising an effective amount an agent, wherein the agent increases the activity or expression of e mitogen-activated protein kinase (MAPK), a protein phosphatase (PP), or a combination thereof, and thereby treating the vasculopathy.

19. The pharmaceutical composition of claim 18, wherein the agent comprises an antibody or fragment thereof, a polypeptide, a small molecule, a nucleic acid molecule, or any combination thereof.

20. A kit comprising 1) a pharmaceutical composition of claim 18, and 2) written instructions for treating the vasculopathy.


SEQUENCE LISTING (16 sequences)

SEQ ID NO: 1 - protein, 338 aa, Homo sapiens
MAKWGEGDPRWIVEERADATNVNNWHWTERDASNWSTDKLKTLFLAVQVQNEEGKCEVTEVSKLDGEASINNRKGKLIFFYEWSVKLNWTGTSKSGVQYKGHVEIPNLSDENSVDEVEISVSLAKDEPDTNLVALMKEEGVKLLREAMGIYISTLKTEFTQGMILPTMNGESVDPVGQPALKTEERKAKPAPSKTQARPVGVKIPTCKITLKETFLTSPEELYRVFTTQELVQAFTHAPATLEADRGGKFHMVDGNVSGEFTDLVPEKHIVMKWRFKSWPEGHFATITLTFIDKNGETELCMEGRGIPAPEEERTRQGWQRYYFEGIKQTFGYGARLF

SEQ ID NO: 2 - DNA, 8343 bp, Homo sapiens
cctcttccaggagcttctgggtcgaggttagggcaacatggggagtagatgtcgtcttgtctcctgaaggctccgtcacttactaagacctagtaagtctactaggaggtgaaccattaacgccaaacgggggcgtcctcctcaccctggccacaaggcgccttcctcagtggtgccctgagttggcgccgtggcgctggttttttaacctagagcctttacttccccctccccccaccactaatgttttttgttgttgttctgaaagtattatcaccacgtgctcaagctcacttttgacaatttcccttcatttctgaggaaggggactggcctgggacccggggaagggcagagggggtaggtaatatgcagaagatgaaggtggaggagtacaagcaagaagtttcatcgtcacccccatgggagggaagcaggctttttcctttgtcacggtctccgaggggcaggaagctgtgcgtctgtctccgggaaccctcaacctccacccaccccaccccacccccgtttccccgccctccggtctccaggctggggaaatccgccgggcctcggccccgccccgccgtcccgccctgctgcgccgccgccccccagcccccaccccgctttgctcagcggtgctgggcgtggggcgcgggccgggtgctgcgcgcggggatccggggcgctcgctccagctgcttctgtggatatgtcgggtccgcgcgcgggattctaccggcaagagctgaacaaaacagtatgggaggtgccgcagcggctgcagggcctacgcccggtgggctccggcgcctacggctcagtctggtaggggccggcaaggacctggtgggagctgggcgggaaagcgtgtgcgaccctccagcgcgacgtggggttcccggcgtggggaggggcctcgccctgcctccgcccccggtctgcacgcttccgatgaatgaatgggggggaggggacacggaggaaactttcctactagattccactcgctggcaggtagggggggtcttctaatgccctctcccccccccccatctccgatccctttcctagtttttccccaaaagactgaatattgcggcgtcacagctggcgggagagcaagtctctggggccgctaaatggaacagtcagccatccgggttatttcgggcaaggtctaagtagctgcagggtctaacgttcccctcagactaagtatttgaaaaggggacccccagcggggtgaggagacctttctaatttggaagacaccaaaattttcctgtggatcatacccgcgcctccagattcctagaagctcagcggcttgagcctaagcggacacctactctcttgtctacaccctaccccagtttacccaacaaaggatctcagaatataggtagggcttggagaccagagattgcagtctcgatcgtgggcagcatccaggcacctgggattggagaacggctgtagatagcccttgttgacgggctgatcaagggtgtggacactggtatgtgccccacaccgggaatgggggctgagtctcacatcttggatccggtgctgtcatccgaagcctcggtactgctactgcaaccctatagaggcaaacagtcctgctttacagggcccagagtgagttggtcatgggatctcgccccatgtctgcagcctgtaaacccaccactgagatgggtaggcttgttctgccgacactcgctccttattcttgcttctcaggctgtaccccagccctccacagtgagtagagacaggatacacccactccatcacccatacatacccaaggagtcggacagaggaagagatgatgggacatgttgtgtctgggatgttcagggactcagcaagtggaggaggataaggttacacgaaaatacattttaaaaaaataatacaataaattagagggaaggagagccagggaggacttggagggggcagggaggaaggtgaagcagatactttgcaggggacttggacattgactcagatggagttgtctcggggctggaccatctaggagttaccagggaaaagccagatttcccaagggagacctaggtctgtctccgctgatccgtgaccctgggtttgttcagtcaccctgctgaggcaggccatgggatgccaacttcttgtaacttgattgtacagaggcagtcaagtaagaggatcctgctgttctagtggccactccccacccacctccaccccagaagttacttagacattctgcttatcataggaaagccacctttcttctacaactctctagttccgtgagcatcccaaaccaccatgccccttccccatcttccctcacctggtgggaatgggtggtaccctgtctccattcaccactgcaagggaagggtgcatcctgagacgaagagaatcgagggagagaaggaacaggaatcaccaggggagcaggctgtgtaaaggtactgtagccagggctccttcagtggcttcgcagctgctccgggtcttgaggcagagggatcagaacctggtgaaaagccttaggaccaccgctcagtccacgtggcctaggccccgcccacaaagaaagcgtgctctcatttgctctttcgaggctctccaggtgaaggatagaagggtaaagaggtgcagaggtggcgggtccagtttctgggggagtggaagtgtgtcagagacatcttcacactggggttaaacatttcctactatccatttgctgagttataaattgtgtgtgtgtgtgtgtgtgtgtgtatttttttgtttgttttggtttggtttcttgtgagacaatgtctcactaggttttccggctggttttgaactcaatctcgattctctcgcctctccctctctccaagagctgggattacaggcatgaacaccacactcggcttcaattacaaatcaggaagcaataaacacctccggataatcttgcttccgcaatctgatgtgcggaggcgtgggggtggggcgtccagggctttgtaggtaagcctcccacggcatccgtgaggaaggaggagggctgcttgctgtgggagcgcctgtgggtacagtcgtggtattttccatgccgtggccttgttgtgtgtgtgtgtggggggggggggggcgctcaggtttgggctagaatcaccggttctctgccagagaacgctttgtccactcaaaggggtctcctaagggaggatcaggagggtcggcgtggaggcagcgcggggcgagcaggcggggacaggcttcctaggttttagagctagcaacagaggctcactcccgcacccagcgtcgggtggattcaggccaggcgcttgctctcctcattcgcccaccttgcagctcggcctacgacgcgcggctgcgccagaaggtggctgtaaagaagctgtctcgccctttccaatcgctgatccacgcgaggaggacataccgtgagctgcgcctactcaagcacctgaagcacgagaacgtgagaggggggaccgaccggtgatgttgggactgaacaagggacgcatcctgcctggggggcgggacctacactataggggcggagcctctggaggtacagtcccatgtgggtggggtccagaccaacgggacgttgcctaaggctaagctgtgatcctgcctcggttccctgaccaggtcataggacttttggacgtcttcacgccggccacatccatcgaggatttcagcgaagtgtgagcgtcctgggcaggcgcggtggtagtctggggagcttagtcggagatggggtggtgagactgtcctaactcctgatcctgcaggtacctcgtgacgaccctgatgggcgccgacctgaataacatcgtcaagtgtcaggccctgagcgatgagcatgttcaattccttgtctaccagctgctgcgtgggctgaaggtggggcatgtgggctctggggtcccctcccccccccgaccgctctgggttgagggcagagcgaggtggcgcctgggctcactcggtctgctctcctctgcagtatatccactcggcgggcatcatccaccgggtaggtgcagctgtgggcaagggtgagggtcatccactctgcctaggctcatggcctcgccacactcaatgcccggtgcaccctgcaggacctgaagcccagcaatgtagcggtgaacgaggactgcgagctgagggtaagggcggttgaggggtgcacagacagtggggccacatagagaggtaggtgggggtcttacacgagatccccgtggtcctccagatcctggactttgggctagcacgccaggctgatgaggagatgaccggatatgtggccacacggtggtaccgggcgccagagatcatgctaaactggatgcactacaaccagacaggtgctgtcaacccttaaagcagtggctttgtcctgtaaggatgagcaggctgtgctgacactgtttagggaggatgacaaagccgtgggggtaggaagaaaggctgaaggggaggagagcatctctggagctctgtgagaggagcaagggattggtgggaccagcttttagaaaggcttctaagctgatgggtgtctgtcctcatgcctcttctcaggacagggtatagagtaaggcttctactggtctctttgggcctggttatgcctgagggtcactgggctggctcctccccaggctcacaggaaggatttggggttggcagactgatagtctccttattctcccccaacagtggacatctggtctgtgggctgcatcatggctgaactgctgcaaggaaaggccctctttcctggaaacgactgtatccttggggagcagggtccagctggggtccccttctccagggtgggagtggactctggccctttttggacgtctttcctctgccctatgccatatcacacattacacggggctccctgtcctgagccacagctctacagtgtaccaggggctgggaccggaaacgctggccaccaggtgtttcctgagctaggtagacatcgaccagctgaagcgaatcatggaggtggtgggcacgcccagtcctgaggttctggcaaagatatcctcggagcatgtgagttagtggccatccgtctatctgttggctcttccgaccaagcaccagtcatgctggggagggagtagtaagcagaaatggcatcgcatgaggaatgtgagcagggagggatggagcttgtactaggaacatccaaccccttcagagagaagcaggtgaaccaggaggccatttcaggtagcagagacagtgctgacagactcccctgcttatagacactattgaggcagggaagagttgagcttttggcttttgtaggcaccatcagaagccaaatgaagtcctggagccccccagacttttgttttgttttgtttttgagacagggtttctctgtgtagccttggctgtcctggaactcactctatagatcaggctggcctcaaactcggagatctgcctgcctttacctcctgaatgctgggaataaaggcatgtgtgggaccactgctcggctagggagcgcccaccctgccccgactttgagagcaggagtctggcttgctggggtacctaccaggcctgttggaatgaatgcatctctctaaaggccttctgcccagtttgggccaggaaaatagaagttagttagccctccaagctcttcttatgtgtctccaggcccggacatacatccagtctctgcctcccatgccccagaaggacctcagcagtgtcttccatggagccaaccccctgggtaaggactggtactggatctaggctgggcttcagatctagcattggctatggtgctaagcctcactcactcctgcagccatagacctccttggaagaatgctggtactagacagcgaccagagggtcagtgcggccgaagccttggcccacgcatacttcagccagtaccatgaccctgacgatgagccagaggcagagccctatgatgaaagtgttgaggccaaggagcgcacgctggaggagtggaagggtgagcacagggatggggcgtggggcgtgaggatgggaaagagagcagtgtccgtgaacctgagggttgccttttctcagagcaagattttacctgtttcatgcaggaactccccagaacttccccaaaggtctgggatggggaggggaagcagggggtggggtgggggaaggaggatggaaacatatctgtttctgagtgagacatgtaagggtgtgtgtgtgtgtgtgtgtgtgtgtgtgtgtgtgtgtagcagaggtttggagggaagtgggcagacagcataatctcacccttcccccctttttcattcccctccccagagcttacttaccaagaagtccttagcttcaagcccctggaaccctcacagctccctggcacccatgaaattgagcagtgaggcattgcctgaagtgggagacctgagcctgtcccctctactcagctttccggctttcctcaagaggcgcctctcaggcagccctgacacttaacctgggaccgacccccttgccttgagaaactctacacacacacacacacacacacacacacacacacacacacagaggaatgcacggatgtgtacatatgcctccctcccattatgtgctggagtctgggcagtagtgtttctggacctaccttggtccacccaggttctccttgggacctccgtgtgtgggatctgagtgtgtccctgtccttgtccaggtctcttctgctcttgaggactatctggattgggaggaaggctaatgggccatggtccctggagactctgagggagggggagtcacagtggttggagatgctcaaccaggaagtgagtggctgaaagcagaggcaagcccagtgtccctcctaactgagtggcgtttcctctggggatgggaaagctgagaccaccatcagattatctatctagtcagaaaagcctgctgaagatttgtggttgcctgtggtatgtgggcacatgggcatgagtatgcaccctgggatacacatcagaacaaatgcaggcatgtgctggtgtgtatgaatctctgagcacccaagaggcagcagccatttctggtctggtgcctaagttggagataactcttgcctcctctccccccaagttcctgttttctaatgggtcttgaatcccagctgtgaactagaccaaaggctctgagaggtgccaagatccattagtttaagcaaatgtaagggtataggagcagcagcaggatcctgagtcacaagggcacaatgccagctggagagctgatgaagttgggggtctaatctcaaggatgctaggtggccagcacccacggatgtgagcatggacattcttgccccgggaccttgattcaaagttgacaaacgtctgtccacgttctacctccaccaacccttgaggttctgcagctggtctggggtgctatggtttgcaagtcacccctcatctccccatcccccagagaggagctgaccaagtaacttttgaggtagggccctgtttgaaaacgcgttgggggtgtaacgcctccctgaagagacaatgatctcttgcttatcagggaccaaggggctctggatgtcaagtgcctgcaccaagagtgtgcacaataaaggggcatccctcttgctcttgtctgtgcaaaaggctctggatcccattgcttctactgattctgcttcctggaggggtggctggatctctgtgcctccgtacacttcctgctagcgccaccgactttgaatttattgtgccccttgatcactcaatgagagcagcagtcatttctagtactatcgcagcttcttgggggtgtgtgtgtgaagaacccaggagaccagtgcaggacaagacacactggttagaaggggcaaaaggtgtaagagccagcacactttggtccctgcacattagggactttgtacattaggaatggtggacagacttggctccctcttactggtgaggctggggattgtccctagagaaaagatctgttcttcctcagggattggacaggatatgggaaatagctttccttcctgactaaggcgaaaaggcaactttagcccaggatacagctcatgtgtcatgtctatgtcctgaatcttggaccgccagagatctgagtctggcaactggtccagcggtggaggttggggccgggggtggggtgggggggtggacctagaaggcaagagccaagtccaaagttgggggattgggagcgggagcaaaccca

SEQ ID NO: 3 - DNA, 11320 bp, Homo sapiens
gggtgggtggaacagaggtgtgaaggagtcttagaagggccgataggataggacccatccctctggatcttagactgggaagttgagagccagtggccacaacaatggggctggggaagcaacctgagacgtttgggagactgaaccaaatccctcctccatatagaaggaggcgaacggatcagccccaggggcggggcatccttgggaaagacggggagttgagccagtaaaaggaaccatttgggatggggtatgattggtgtatgggcaggtggaccagacctggggcttgagtagaacctggtggtctcaagttgcctagaatgggtccgacgttctgggaggacaggaaaggctggaattaagagaggcacctgctaggacccaagggccaagaagtctagcgagggtgtaggctggccagggtgtttcaacgcctcctctcatctcaggtcgagactgagctatggagcagctctcccccacaaccccccctttccaagccttccgcccaaccccggaggcctcctcgggtttccgcaggggccgagggcaggcgggggtgtcacgtgctcagggcctgggggccggttggtcagtgggccgggagggaccactttgcgggccagagacaagggtcgggtgggagttggcggttatagagagttcagggtggcgaccccgagagaagagcccgcaaaggaaaatctcagaggcgggcaggcgggtagccggcgcggagtacgccctgccacgcagtgacccggggcgcgcgggcggagcccctgatcccgggtccggtcctggggcgcggtgctccggctggggatgagctccccgccacccgcccgcaagggcttttaccgccaggaggtgaccaaaacggcctgggaggtgcgcgccgtgtaccaagacctgcagcccgttggctctggtgcctatggtgcagtgtggtgagcgtttgccgggcggggcggcaggcagggcaggaggcagctggcgattacggggccggttcgctagccaagccgtccgccccgcagctctgcagtagacagccgcactggcaacaaggtggccatcaagaagttgtaccggcccttccagtcggagctgtttgccaagcgcgcctacagagagttgcgcctcctcaaacacatgcgccacgagaacgtgagtctaaagcccgccccagatgcggatattgtgcgctggcctcttctcatcctgccctcccctgcaaatcctgcccacagtctctgcttccaagggcctggtacctcctacctagttagctcccagcttgggtaaactttcaacaactttcaggaggaaagcagaggcagagcccagatccctttctgggtgacactacaatttcagcctcttcttcaattgcttggtttcaccttcggtccacctctaccttcgcagcccaggcagtggtcaggttatggtctaggcctaaggattcctcaggtctcactccaaaacctgattggacaatcaggttggtgtgaggagggttttcctggaaggggtacttccttaccctaccctagtctccctttgcccaccacccgagtcttctattccagacatacaggatcctaggcattcagggcaaggcaggatactcgaggtgtgcagccttggctctatcatcccagagccttgggtctctgtggataaagggatttacggagcagagcatcgccattagtgttgtgggtgtgtatttcttcacacccatacagaaggcatctcctttcccgtcctacctgcatatggctactcctaagcctcttgcatcaccattgctagccatcagagcattcctcccaaccagggacattattgttattgttgttgttattattattattattattattattattattagatatggttcttcctgaacctgtgagttctgtccctcccacttctgttctgtacagctgccctagtcctgatgtttcaatcctcctggatcatcagagtcaatagctccaaggaaaaatgaacatctcgtggctaacagcaaactgagccccgttgcttcctcattgctactcaactgatttggtggatggataccagccctgctaggtctgcttctttgcttcccagtaaagctggaagcagtaaacaaacaaacaaacaaactcagtaagcctggcagtggcagcacatgtacctgggggggcagaggcagatggtccccatgagttcgagtccagcctgatctaaagagttagttctaggacagccagcttctacacagagaaacccaaacccaaagccaaacaaaccaaaccaaacagtaaaaggctgtgttttcttgggtgtaaccacccctatcctcagagcccccaacttccacctacttacttatcctgaacccttcttgaacggagagctgtggagctcttcgttcactccccacttctttgccagtctgtgggtcacccctgccttgtgaagagcctttacatcaagctactgacctgtcctcacctggctgtggtggctttcaggatgcctcaacacatttcggggaggctgccattgtaacctacccttccatgtggaccctgaggtttagataggctgtatgtctcactcacaccagagatggggttgccacagggccagccctacccactgctgccgccgccgcctcctcctcctcccccccactcctcctcctcctcctcctccttctcctcttcctccttttcctcaagctctagtccttggctctgggtcacattctataccacaaaagtgcagaattcataatcctttgaaggacctgccttgacctccggcacactattccttatctattctgcgctcagctcatgggtcacacctgtgagttatctgtctccaggcctttcctgtccttgctcttcccgctagctttggccatgactaaagcagtcgcccatctgccctttacctcctgtctattcttgccgcattctgtcacagaaactaggagggcccatccagcatcatatccttgtttaaacctttccacggctgctcgctcaatggagcaaagccaaagctgcccctgggatgggagtgtgaggccccccttgggcttgctctgtcctttccttgattccttcttggcctgggcttcggccacttcctttctgtaccttactctgtagggactttcactcctgtgatggccatcaggcacctgacctctcatttgctcatttactcatccgtcctgggatttggatatatgcggccctcctcaggacaaactcccttgaccccggaggtcccgtcccctaaccaagtactgggaagccaatgtcacattgtctatctgttgtccaactccctcaatcaaaagctgtgtttcaattggtgcttggtctagacagccagaggggatggtcccaggaatgacctactgggaattcattcctcccaggggacggtccccactttggtcttgggtctgcttcattgggaagaaaaaaagtgtgtgtgtgtggtgggggggatggggagacaggggagacagaagactaaggggaccggattgctcagaggcctggagccaacccagacctgagactgcccctccccccaccccctgagaatcccggctgtcagcctccaaaaatagcctgtgtctgtgggcaaatgtaccaggatgggggctgagccgacagtggagacagtagaggagacagggatagttatgggagggaggattttcagcctggagtacagtgccatcccgggggtgcactgaggccctgcagctcccgtgtgctctctatcccgtctgctacttgttcttcaggtcattgggctactggatgtgttcacacctgatgagtctctggacgacttcacagacttgtaagtgctagtggctgagagggtggcaggaacctgggtggcttctcggtgtcagcttaagatcctgggtcaggttgaggaccttctccccatagcccagcggttctcaaccttcccaatgctgtgaccctttaatacagtttctcatgtcctggtggcccccccagccagaagattgtttagttgctacttcataatggtacttttttttttgggggggggggtccgagacagggtttctctgtgtagccctggctgtcctggaactcactctgtagaccaggctggcctcgaactcagaaatccgcctgcctctgcctcccaagtgctgggattaaaggcgtgcgccaccactgccctgcataactgtacttttgctactgatagaaattgtattagaaatgatatgtaggatatctgataagggacaagcccccctccccccagggagtcatagcccatgtaaggaatagggtgcacactgcccacccactcctcttaccacagtctataccccactcctatatgctcctgggagccatgaacttgtgtgagtgataataccatttttccttgtgtatccctccatctatcttaacatccctctgacctagatgccttgccgctccaacccctgccccatattcctggttaccctatgtgtggcaggaggggcctaggcagctcctgggtaccagcttcaggttctaagttggcttgagttcttcttcccatggcccaagtgaggactgagatgtatacctgctcattcacatctcatcataacccatgtactattggtaacccctggatactgggatgccctagtaaagccagactgtctatccttctaagcctggggagttttgggtcctcacagtgcctgaggtattggtataactaagcaatctaaggtgccaatatcaggtgcagcctcagccgctcaccctgcatgttgggaattttaaaaccctgaggtggcctgtggtggcacacacacttgcaatcccagcatttgggagatagagctaggagggacaggattccaaggctattttggcctgcctgagaccctctctccaaaaacaaaagcaagggctggagagatggttcagtggttaagagccctgactgctcttccaagagcacgtgggtttgattcccagcgcctatctggtagctcacagcttctgcaactctagtcccagaagagttgatatcatcttttggtctctgtgagcactgtacatggcgtggtccacggatacacgtggaggcaaagcatccaaaaaataaaataaagacaagacaaggctgatggtagtggtgcaagcctttaattccagcacttagatgacagaggtagaggcagatagctctctttgaatttgaggccatcctggtctacacggtgagtttcaggccagacaggactgcagagtgagacctttcctcaaaaacaaacagagagccctcaaacccaaacaaaatacccctgacacacagcccactccttgcttgccgttctggaaagaggggatgtgggcatgggttgagaggtgtaattgtcaaggacagtgtgcccagagggtgggttgtggtctagcacggcatggtaggggtagaaacatcataggatggtatgacccccccccccccattgctagtgatgaatggacactctagggtaatgatttggtgggcttctgcagctacctggtgatgccattcatgggcactgatctgggcaaactcatgaagcatgagaccctgagtgaagacagaatccagtttcttgtgtatcagatgttgaaggggctgaaggtaggctttgcagaggtggccatagggaggcggacagagcttcccgtttgggcagggtaagggctctgtggcttggagtatccctgagctgactgctgctgaactctttctttcccccagtatatccatgcggctggtgtcatccacagagtgagtcctggcggggctgggagattgcagctgggctcctctccagctgtgtagacagatgtgctcaaccccagtgcccatggcttaactatgaagggcaagataggtctgtggtggggatgctttccctggggaccaggaaagcccctcttgctcttccccagagggaccaggccagttttaggtggccctaataatgtgcagcctgagccctgctctggcaggtgggtcatagtggccacctttcttctctcaggacttgaagcctggcaacctggctgtgaatgaggactgtgagctgaaggtatcgctgggaggggaagatgaccacggaaggggttgggggaccccttctacttcccctatgggtctgcttgacccttggccgaggcaaagggacagaactcagacatttgtgcagaggctgagcagctattaggcaaagagtggctgaatatccctagtcactcagggctcctaaaggtgattaggggacggtgaaacagaagaaaacctttggcatttgacctgaggtgggtagggccaggtgtactgaccacagggagaatcctgggggcgcaagaccaaagggtggcaaacagtttagcgtagaaactcaagcccagctctcttaaggtcttctggaccatatctggtctatcctcagtgtctaagttccacctcctcgatacttctttagatcctagactttggccttgccaggcaggcagacagtgagatgacaggatatgtggtaacccggtggtatcgggcaccagaggtcatcttgaattggatgcgctacacgcagacaggtaagaggctatccagagactccagtgcacaccatccacatttacacattcattgtattttaaaattatttttgtagtatcggggatggaacccagagtcttattcatgctagacaaatgtcctgtcactgagttacatctcagtccgtctgtctgtctgtctgtctgtctgtctgtctttctttctttctttctttctttctttctttctttctttctttctttctttctttctttctttcgttctttcttccttccttccttccttccttccttccttccttcctttctcttccttcttccttccttccttccttccttccttccttccttcctccctccctccctccctccctccctccctccctctctctctctctctctctctttctttctttctttctttctttctctccttccttccttccttctttttgagaaagaatatatatgtgtatgtatgtatatgtatatttggtatggtttggttgctttgaagacagggtcttactatatagctctggctatttctggaactctctgtaggccaggctggcctcgaactcacagagatccacttgcctctgcctccttccttccttccttccttccttccttccttccttccttccttccttcccccctctctttctttttacctaccttccttccttccttccttccttccttccttcctttctttcttttttcctgtatagatcctttatagtcctgactggcctggaattcaatgggcagaccagactggccttgaatccatagagatctacctgcttctgtctgttaagagctgggaattaaaggctgtattaccatgtctggccccttcttaattttttttttttccgagacagggtttctctgtgtatccctggctgtcctggaactcactctgtagaccaggctggcctcgaactccgaaatctgcctgcctctgcctcccaagtgctgggatcaaaggcgtgcgccaccacagcccggctcttaatttttataagaacttattttatgtatatataagtacgtatctgcatgtatctatacatgcagatacatgtatccatacaccacctgtgtactcggtgcctgcagaggccagaaggcggcattggattctctaggagtggagtcacagatggctgtgagctgtcatgcagatgctggaaaccaaaccaaggtttctggaatagcatcccatgatgttaactgattagccacctctagctcccttcatctctggatagggagccatttataggcactacctctgggaggtctgccacccttctcaatgacagcctctgctctgtgcctatgtgcacgtctccccttcatgtatacttgtctcccacgtgtggtctctttgtgcatcccccttttcttggccttcacggatatgaatgccacccttgcatgtggggcactgatgggacttctcagtggacatttggtccgttggctgcatcatggcggagatgattactgggaagatcctgttcaaaggcaatgaccgtatccttctatgtagagcacccgttgacttcggccaagatgtgggtgctgggtgggcccccctctgtccctgcattgtattcctgaccttggtacagacctggaccagctgaaggagatcatgaagatcacagggacgccccctcctgagtttgttcagaagctacagagtgcagaggtcagtgggagtgggtggtgggctggacttgggcttgggggctggcctatcaccctttttccctcacaggccaagaactacatggaaggcctccctgagttagaaaagaaggattttgcctctgtcctgaccaacgcaagccctcagggtaccgctggatggggtgaaggtctcagggtactgggtgagtatgtcccctcctgtggcttggcctgaattgctcttcccacagctgtgaatctcctggaaaggatgctggtgctggatgcggaacagcgggtgacagcagctgaggcgttaacccatccatactttgagtcccttcgggacactgaggatgaacccaaggcccagaaatatgacgactcctttgatgatgtagaccgcacccttgaggaatggaagcgtgagtgtgggggttccatacaatgtctggctttggaccctgtgtgccctgggtgagtgagcctctgcccctggggtgcaggtcagataagagctttgggaggtcaaagggcattgtggggcagtgcaactcaaccaggttgacttctggctaggggggaaggcagtggtaaagagtgtcttttgaaggagctccgacccacaagggctggcaggtgatggatgctgagaactgtggagcaggtgtgtgtgctttgtgcccttggcttactaactccctggccgttctctccccaacctctatattgcctagcatcctcatgtccagaccacactggagctctctctaccactgccggtccttatgactttcttcttacacctttaaaatgtatccttacctctccttactccatttaaagacctctcctgaaataactatatcttatgtactgcccatggaagacagtctattcccatgcaggccaaagggcccagcttttccctctcatctctagccctagatccctccatagctctgtgccaaacctccaattctctctttgagaactggcctcagagaactctctcagtttgcctactgtcccctgagctgggactagcctcccgtgtccttgccagactgacaggtccaacctggggagccaggaggtttgaggacaggtccattgcgacatgctatcccttgtcttgtctgcctcagaccccaggcccttgttcttgcaccctggtatgggaaagactggatgggtagaggttcttgactcatgtcctgaaagtcctggcaggtgttcttcccttctgtgttgtcgtgtgcttatgagtttggttctgtgtgatgaaatccttctgctgctcaacaaaataactcccctaaccacgggccacaagatccgttctgtggtctccatctacacactgtggctgggtgcttttgctttcaccatagaccacaggaggcacacaactgagcccttgagcaggctcacacactttcttctcacttccttttttttttaatacaagcctctctctcttattattattattttttgaacacaaccccccctttttttcttttgaacatacactttcatcccttgttttcctggggcaatctagcttcacagcagtcttcagtgtttttaagtggattggcccacttaggattagatttaaggcccatggggtgctggcttccccccttagttgaaagctgtctaactctatcgtttgcttctcttcgcttcccgcaggtgtgacttacaaggaagttctcagcttcaagcctcctaggcagctaggagccagagttccaaaggagacggctctgtgacgacctctgggtggtttggggggtatccaaaggaggttggctcggagcttcacggcaccttggcttcccttctctggaaaaggaatcctggttaacaccccgacagtgcctggagcttgtatcccaagtcttccacctggacatgctgtgtagacccttgaatcatgaaccctccatctccaaacctgttcttcggctttcgagtgccccagatgaccctggaagaacatctaagctttctgtccaagacccctacccaacatgggactagcctttgaattctggagttgtacatgaaatcagtattcgtgaaaaagcttcagagtgagcagagcttaggagacaagtgccagacctgagctctgctcgctctggacaatgccaaggccaactcctgagacggaatgagacagaggttttggggacactgactcagggacatcatctcttctggaagtgggtggattctcttacacccttagcctggaattcgaaccagccatttggtgttgcctaagtggctgggggcaaataaacccttttgtagatctcccattctggttgtgtggccccatgccttgagcacccatcagttctccaagctgctctgtgcacaaaaaggagctaacctgagctctatgtcctccagctaatagaggtgccagtcccctgactcttcccaggccacagtgctggtgccacttagtccttgccaatgcttcaagccacattagggcttggcccctcccttcttgaatgactgtactcttaaggctcagagtctgagcaggaggtctggagactgatgcaagggaggatggtggggaagatgatgttaagacccagatagagggatgcctccttctttctgagtttgcagcatttgtcttccaatggtccagacttctagtgaggaaggtcttagctctctaccagcatttccctggcagcctccctgaacccatactgtcatctgcaagccattagacacgagaccacacttgggctggttagatggtccatgttcttcctcatgtcccaaggacaggtgcgacagggcaggacataagggtgaggtctttcattcccttcatttgacaagttctgggcaaatacactttgcccagtctaatcttgtcagaggagatgccatga

SEQ ID NO: 4 - DNA, 12137 bp, Homo sapiens
aacctatgctggtctccagtttactgcgtagtcaagaatgacgttatttctctatccttccttcctccctcccttcctcccttcttctccccttccctctcttcccaccctctctatctctccctcctttcttctatccctctcttccttctctgttcattctctgtatctctcctctctagtctctcctctcccttcttcctcagggtctcacctagtctggcattgaactatgtactgaactggactcaaaatcgtagcaattctcctgtctctgcctctatctcctgtgttgagtacagacctgagccaccacgccttacatgtgaagggtatatttcttaaactagcccaactagcaacaagttgagcgcccccccccccgcgccctaaccccattcccaactcttcctacagtctctaggcagcctcgcaggcacccctttcccgcagggcacccccctcctccctcagcacctgcagcaggtggctggtggggcctggggcgttcgccggtgatgggcggagtccggagcgcgggcgagcggtgggcgggccggggcgggaacgtacctgggcgaggcggcaggtgcgccgccccacgggggaggagcgggcgcggggcgggaggggaccgcggcgggacacccgggccgggagcccgcgggcagccaccgcaacctcctcgcaggaccgccaccacggggtccgggatgagcctcactcggaaaaggggcttctacaagcaggacatcaacaagactgcctgggagctacccaagacctacctggcgcccgcgcacgtcggcagcggggcctatggagcggtgtggtgagacccggggaccccaggaatcagaggagccacgggccacccctcggcagcctccccgagtgagcccggtggcagtggggcccctgtgggttgagtcgctggcgaatctggttctccaaggctgtaactttttaaaagggatctcggagttcgtgggacagatccctgcggttgcagatgaggccccaagaggggaacttgccaaatcgcagagcagggactcctggcctgctctgcatcctgtcagcaagaactgcttctctccttgctctgccgccccttccttggcgtcgccccctactcacacacactctctctctctctctctctctctctctctctctctctctctctctttctgctggtctccggccagcctggggagcacagaatgggggtggcctaggacagggctggagggatctccagtttaggccctttgcgccgcccctccccctcacctgcctgtgaccccgcagctcggccatcgacaagcggacaggggagaaggtggccatcaagaagctgagccggcccttccagtcggagatctttgccaagcgcgcttaccgcgagcttctgctcttgaagcacatgcaccatgagaacgtaggctgcctcggggtgtggatgtgccggggatcggagggcagggagaggggcccaggaggggcctgcccagggtcgtgcgcgcagggccaggagaaagaacttcctctgctccaccctggtttctcagctctctaaggagagcccaatgagaagatgccagggcctgcccagctcgcttcccagaaaacacacaaccttttcttgattgtgaggaggaagtaggcacaggggagcttcgcgattggttctgtgaggactccagtccgatgagcctgggcacttgagctaaagctgcctccattctggccactctaagtctaacaataaatactgagtatttaatgcatgcgggggtgggggctggctaacttttataatcctctccgtaacacatgccaaggcacagaaaggtttccccggttctgcagcggcagagctgaggttggaaggggcgggaggcagagcctggtgcctgaccatttgaccatcctgtctgatggaaacagaccccgtgacccagctctgggccaggcagaagggctagggtccatttcaaatagagccttgtacatcgtgaaccctaatcctttcagataacctttcctcagtggcctccagcttcgaggtggaggggacttgcctctagctctggacagactgacagatgtggctagagacccggggtccaaggctagagctgaggtccagagtcccaaggacctggagttctcatccttctctcctcttccccccttgaccctcaagatttgttagttacaagtgggactgttcaccccaaaccccaaatttgggatcaaacttaccttgttctcagccatctctccagcccaggcaaatgtcctcttctgcctgggcccccaaactgttcccatgccagctggggccaccttccgtagcagagggtgacctggatggtggccctgggtggctgagaccctcaggacagggaagaatggcgttggcagcgcaccctcactggtggcctctctccaggtcattgggcttctggatgtcttcacccctgcgtcttcccttcggagcttccatgatttgtgagtcgggctggattggggtgttggcagatcttaaggctggctcgggaatactttggggcagggtagcttccgcgcagaaaggggtccgtccccccccacctagcagttcttgtgcctctgcttttacattgagatcttccctggagaggtcccttggctgtgggtggaagggctgtagggtacctgccccccccccccagagctgtctacacagctcctacccttgttatcagaccctggcaccaagaagagctgctggccctcacagggtgtgtactgactctcctcacccattcctggtggccaggtgtgactttggacttcatgatacctagagggtttttttttcccctagaaggaagtgaggatcagatagcccggacaatctggctgatggggtttggctgttacaaagtgtcaaagtgtccggttttgctggcaggtgcctgggcaaggatgggtgtttggatgttcctgagaattccaggtgttagtggcagcttctgccaggcttcacatcctagtcaatgctacagaggctgcgggagtgttttggtaccttgtgtgtttatgtgtttaaattggacctctcctgtagcccagtctggcccccaactacctatgtagacaaggattgcccttgaacttctgatcctcctgcctctgattctcaagagctcggattccaggcactggtgctgagaatcaatctcggggttttgtgcgtgcttggcaagagctctaccagcctagctatgtccccagcccactaagagtggaaggtgttttgttttgtttttttccagacagggtttctctttgtagccctggctgtcctggaactcactctgtagaccaggctggcctcaaactcagaaatccgactgcctctgcctcccaagagctgggattaaaggcatgtgccaccactgcccaacaagagtggaaatcttaagcagagtgctgatactgtctgtcagatttggagggcatggccagcagttctgagaaaagagcaagaagcccctgggaagaaacaagtgctgagagcccccccccacacacacacaccttgatctgagttacagtgtggggtccccagggcagacctgaggcccactcaagccaagctgacagagctatgtcatctgtttggacatggtggctgagggaggttgggacaggtgactaggagtcccaatccctgcctggaatataagtaggaagaagagacctcaaggaagagataatgaacttgaccttgggtccttgactcttccagagcctgtgaaatcacaggaggggggaacatggatgtagaccaggctagcatcctctccatgggtgatggctaaacgctggggactaaacatggtaagacaagatacaaggatgttggtccagagggcacatctgtgagaaggcaggaagggtgagcaggtgagaactggagagagaggagggctggcaggcacctgggagtcaacagaaggacacacaggaggagtcactgttcttgacgtggtgactgtgaccttgagcccaacatattaggacagacacaggtaccatgactaggttatgagaggcagtgtatttaggtggtggagaaagtgagataaactgtatatattgtatattacacatacacacatgtacacatacatgtacatatacatgcacacactctcacacatatatacatatacacatatataaatatacatatacatacacatatatgtatacatatatgtgtgtgtgttggtatgtgcacatgcttgaaggagcccctgaaatcaaagacagttgtgagttgcctgatgggggtgctgggaattgaacttgggttctctaggaaaactagcaggtgctcttaaaagctgagtgattcttccagtgtccagcaatagctttcagaagcaaagtccacagcaaggtagtataaatatgtaatcccagcgctcaagaggtaggtggagccctttgggttcgaggccagccttgtgtacatgttccaggccaggcaaggctacatgatgaggccctgtctcaaaataaagggaaaaaaatgtgtgcatgcaagacacatgtgatcgtgtgtgtgtctatgtgtatgtgagagagagagagagagagagagagactgtgtgtgtgcaagtgtgtctgcacgcgcatgtgcatgtgtgagtgcttgtgtgtgtgcacatgtgcatgcgtgtgtatgtgcatgtctgtgtgcatgtgtgtgagtgcacaacacacaccttttataatctattacttttttttttttttttttttttttggtttttcaagacagggtttctctgtatagccctggctgtcctggaactcacaactcacttggtagaccaggctgggctcgaactcagaaatccgcctgcctctgcctcccgagtgctgggattaaaggcatgcgccaccacgcccagctaatctattactttttgtctttgtttttttatttttttattatttttattatttttttactttttgtctttaacgttgctcttaccaccttgagtcacacttcccggttgctgaccctgacctgtttcacttgagaacctaatagctcgggtcccttcttctccaccatctgaagattgtttcctgcttccttcctggcagggcatctgttttatagttagcagagccagctggggtgggtgtgaccctctgcaggagaaacttttggttagaggtaggaatgctagcagatgctgtgggcttgctgctggtcagctatgcgaggagggcagagccagggtacctttgtatgaacgcaggcgtgtggtgatacaaagtgtgacctagtctgttgttcaccctgagctcggccagccaggttgctgtggccgacattgcaggagcccaggctgggcactgctcatttttcatgtatcctctttgtttgttttctgtgttattgtgttcatatgtagggagctgcatatgtgtatgcagctatgtgtacattgtatgtgggtactgaggcttcaactcctgtcttcctcatgtcctatttacatatgtgtgcttatatgtgcatttatctggaggtcagaggtcaacctctgatgttgttattccttaagtgctatctaccttgattgtttttcttatgattgtagttgtgtgtatgtgtgcgcgcacgtgtgtgtgtgtgtgtgtgtgtgtgtgtgtgtgtttgtgtgtgtgtccctgcatgagttttgtgcaccatatgtatgcagtgcccacagcagccagaagagggcaccagatcctctggaactggagttacaggtgagcctcaagatttggatgctcggagccgggcgtggtggcacacgcctttaatcccagcacttgggaggcagaggcaggcggatttctgagttcgaggccagcttggtctacaaagtgagttccaggacagccagagctacacagagaaaccctgtctcaaaaaaccaaaaaaaaaaaaaaaaaaaaaaaaaaaaaaagatttggatgctcggaactgaaccagagtcctctgaaagattaaagccatctcttagtccccttccatttttttttttttttttgaggcaaggcctctcactgacttgccaactaaactaggcttgctggctaatgagccctggggacccttctgtctcctccctaccagcactggggttacagctgaactccaatacacccagcttttatacgtgggtgctaagggttgaactccagacctcctcatgcatctgagcactgtgtcagctgacccagctctccagccccgggactcactatgtagcccaggctgggtttaacacgttgcaatctctaagtgctgggattacaggcatgaactatgacacctgagtcgtcttctgccttcttgcctggtgttgtcttcaaggttactgttgtgcccgacttctgtccccaccccaccctgacctctctgaggtcacacctgggcctgtgggaggctgggaactgtgctaaaaaacactcctgccttccagctacctggtgatgcccttcatgcagacagacctacagaagatcatggggatggaattcagcgaggataaggtccagtacttggtgtaccagatgctcaaaggtctaaaggtgggtggagcctacatggtagccaaggcaacttccttgttgagagccagcccgctgttgtggggcagaggagggttctcgatgctggtgtggaagctgacgtctctgattcacatctctctctctctctcattctctctctctctctctctctctctccctccctccctccctccctccctccctttcagtacatccactccgctggcatcgtccacagggtgagtgctttctctatcatggttccggggaatgagttttggggtaggaacagggtactctggtggccaagggattgggagtgacagttctcaggagacagaggctgtgagtgtttcagccgtgagagggctgatgtagaatgttgagagaatggctgtgttgagaagttggggagatgtttgtgattgtaggtgtgtgtgtgtgtgcgcgcgtgcgtgctgcccccaaaacccctagctggcacttcaggctctaaggacaggaacatggtggaaaaggatgtgtagcccaatgtcattttacagatgaggaaactgaggcctgaaagagaagtgagctggcccagctcctgttgcagtgtagtgaaggatgtgtgtatgtgtgtacacgcgagtttgtgtatgcactcatgtgcgtgtgtgtgtgcgtgcatgtacccatgtgtttatgtgtatgcacttgtgtgtgtgtgtgtgcacgtatgacagatctgacaccacctgctggctccgcctcagtctgatgttccctttccgtcgtcccctcccaggacctgaaaccgggcaacctggctgtgaatgaagactgtgagctgaaggtgggtcacagcaggcccaccaaggatggggaggtggatagaattgtgggaggtaggctctgtccctcagaacccctttcctcagacagcttgttatccctgtgtcccttgagccatagatcctggactttgggctggcccgccacacagacactgagatgacgggctatgtggtgacccgctggtaccgggcccccgaggtgatcctcagctggatgcattacaaccagacaggtcagtgcttggctgccctggcagccctggtgacctacactggaggtctccagacagttcagacaacacttttctttccttccagtcgacatctggtctgttggttgcatcatggcagaaatgctgactggaaagacactcttcaagggcaaggactgtatccttgaagaattattcattatacgtagttttattttttcgacagtagtcctggctggcctagaacttgtgatgtagttcaagctcgtctctgcctcctgaatgctagggttaaaggtgtgcactgccacacctgacttagtgagtgtcattgtgaaacggggtctcctgtgtgtgtcccagagagcatctcaagctcatgacgtagccaaagatgaccttgaactgctccctttcttcctgacacctcccaagtgctagggtcacaaacgtgtcactatgccggggatagaacccaggatttcatgcttgctaggcaagcactctatcctctggaggcccagcccctagaatggttttaaagatttatttttatttatgtgtatgtctgtgtgagtgcatggcgtgtgtgcaggtgctctgagaggccagaagagggcaccagatcccctggagtgggagtcacagggttttttatttttgttttttgttttttaagcactggggcatgggaactgaacttgggtcctctggaaaagcatcaagcactctccagcccctggagcagtttgaaatgcagataatcaaaagataatgagtcctttctgcccccacctctgctagggaaccctcaggagccaggcaggggcagcccactcagctcagaatgaagaagaaccaggggaggggaattaggcgtcgtaggtctaagtagcctccttctgacctgggctgtctgtcgggaagccctccagggacctgctccaaccgtgccaagagcctgccgtggttaggtggggctggagccaggagtgtctgccaggctggctagagccaggctagaggcagggcaggcaagctcagaacaccgcagagagcaaggctgtgccttaactggctgccaagacctggaccagctgacccagatcctgaaagtgactggggtgccaggtgccgagttcgtgcagaagctgaaagacaaggcggtgagtggctctgactggctctgactggcaccttcttatgggctccctctgccctggcagaccctcttttcctcatcttatcctggactttcgacagggaagtcatactttctcccctaaggccagaacctgaagacattccatatttttctgtcttccaggccaaatcctatattcagtccctgccccagagccccaagaaggatttcacacagctcttcccacgcgccagtccgcaaggtgagtcctggctgcgatccccagagtgcttgcagcccttcctagcagccctctgtcccatgccaggtgtaaggctcaccaggtgcggggctcactaggtgcaaggctcaccaggtgcaatgctcaccgggtgcaaggctcaccaggtctggggctcaccaggtgcaaggctcaccaggtctggggctcaccaggtgcaaggctcaccgggtgcaaggctcaccaggtgcaaggctcaccgggtgcaaggctcaccatgtggtctgtccccgcagctgcagacctgctcgacaagatgctggagctggatgtggacaagcgtctgaccgctgctcaggcactggctcaccccttctttgaacccttccgggaccctgaggaggagacagaggcccagcagccttttgatgatgccttagaacatgagaaactcagcgtggacgaatggaaacgtaagagatgttcccatcttccctctgcctgcccaccctccaggcctaaggggacacacctgtccctggtagctgtccatctcctgtggggttttctccttgagctggtttctactctactatctgagggtgaactctcccaggcccatcctaaagcgcagttactttccacagaacacatctacaaagagatctcaaacttcagtcccatagcccggaaggactcacggcgacgaagtggcatgaagctgcagtgattgatggctggcctccatgtagcccaggttggcctccatgtagcccaggctggccttgagtcttcctcctctccttttttgagacaggatcttactgtgtagccctggagccctggctagacctgcaactcaagagatccacccacctctgttacctgggtggtgggattaaaggcatgcaccactgcgcccagctcaatgccccttttctgttggttttgagatagcatctctcaaactcaggcacttcagcactaggaagtcttgtggtctgcagaagctttccagataaagtcacatcctgaggttcagggggttgtgaactggggtggggggtggctggggacaataacttgctactgatggtgcaaaggaggccactcctggaccccttgttctgttctctgccctgttagggagccaagtatgttagatcccaggggttgcagcgcagcaggctgttgctggcaggtattttggatgcctgataagcaatggagacaggagaccagggcgggtcagtgagtgcagcagcagacatgggttcttcctggctaaggttgcagacctctgttctttccaagttctttccagtgctctgcactgagaagaagcacctcagagtcagacggcagtggcagcctgactcctccttctgctttggtagaacgagttcagatacccaggcttgtttgggtttctgccttgggtttctgacatctggtatcatgtgcaccttaagggaggaacctgatttcaataacacacagtgtatctgaggggccaaagtagaacccttctgctgtctgtgttccctcttccctgggtggcctgccatgggactcatgagtccgaggatctactgtgacactgacccctacattcaaggctgtgacaagtcctgcagaaaagacctctgttccctatcgtttattccagcgctttccagtggggcttcactgtcccagaggctcagtgaaggcagttaagtcgggtgttttgttagaatctgtgccatataaagtagacacgctggtgaatggcaggaggtgagttacccataatgcaccgttgttaggccatgtcacgtgttcatgcaagacagtagccgggggctggctgggggcagagggcagtgtcctaggatctgctctactttataaacatgaagcacccaggaaataaagcttatgtgacggcaacagagctcctcggcctcagagatctgccacagatgccacagtggaggctgcggcaggcaggattggaacccatgacatcagctgcacctggatgccaggcccacctacacaccatctgtagtactgcctccccaagccctgtcactgccagttgtgtctgtactctgtcccccccaacccccatctgagtggccatgttgtcatatggggaggagactttgtttgggactgactggtggccagctatcaccctgaccttacagttagttgcaaggcaggctcaaggttccccgtttcaaacatctcgttttgagcctccctcttggtcccagagaagaggcttggtcttctgtctcaacattcacacctcagtcatttttctcagattttagttgtccctgctttccagattcctggatttccagatgccagggcggccagccgaaaggcctaccatggacactgtcagctgagaggctctgacaagccctgaagaaaataaaggttttttttaagctttatttattattatatctaagtacactgtagctgtcatcagacacaccagaagagggcatcagatctcattacggatggctatgagccaccatgtggttgctgggatttgaactcaggacctttagaagagcagtcggtgctcttaaccaatgagccatctctccagtcccagtatttattttgaggcaaggtcaaataatgtagttttggatggcctggaacttgttgaatagaccagacagactaaccaggagctcacagagatcctcctgcctctgtcacctgagtgctgggattgaaggcttataccacacacacactctctagatttgatccagacccttcctaaagatacttcaccacaggggcctattttcttgtggcagtgccacacagccctctgcctggtgtagaggacccattgtcccccagcagacaatgcagtacggaccttggtctcctctccagtatgaactctgtgggaacaaatgcccatgagtgagtgaatggaggcattatgggtgtccccaagtcccaaagactatttctctgtctagccagtcctgcggaagagagagagccatgggaagcacaggggagat

SEQ ID NO: 5 - DNA, 58278 bp, Homo sapiens
tgtaccttccccttccagtagtgggtattgaacccagggccttgcaaatgctggtccccagcctggaagtggccattcatgatgtctacctctgacctcgtgcattggaactccctgtgggtccctggaacctccttctgccacttctgcctgaaaacatgtaggagcgcctggagggagagcctgggctttgaggcaagactaatatttgcctagggggcaggtttcaccagcatcccgaggtgagccacttgtccttgagcagaccggaatgcgacagctttctggacagggcctgtctgttctttcaagttgttcttggaaattgacgactctagtgagcaggttgctcggagttcaaagatcatttggctctcttccccgacgctgtcacagcggctttccgccatggcgccaggcattcgcccgtgcgaccacagagaggcgcgcgcgccccgcgctgcggcctccacccttggcgccccggggagcccagtccccgagagttcctgcctcggctccccaagccgcgtccctcttctcgcgagatctgccggccctgcgcgctcttctcgcgaccccccgccccgtgggcaggttccatattgggtaagatctggattcagagcggggcctccttggagctgttctcgcgagagttccgcgagaggctcccggccgctgcctgtgggatcgccgccactggagcccaagcggggcgctgaagcgcgagcgggtgtcttgcggcgtcggcgtgcgctccctccccggggagcggctgcaggaggaccgcggcgggagcagcctcgagccgtgcagccggctccggcaccttgccgacgctcgtaggagccgccgcggctgacaggggcggcgggtcgcagcctccacacctgcgcgggtggcgggcgcggggtccggtctgccgcgggcgggcgcagaggagagcgtgcggctgcaggcaggagcccccgctcggccacctcctcgccccgctgctgccgctggaagatgtcgcaggagaggcccacgttctaccggcaggagctgaacaagaccatctgggaggtgcccgaacgataccagaacctgtccccggtgggctcgggcgcctatggctcggtgtggtgagtgtcgccgggcctcgggttaggggtggggtggggggacgcggtggagcaagcagggtggccctgcatgcctgagggccaggcctgtccccacagctcagcgatgcgcccagcggcaggaattctcctccgggcgtgtctgggggtgcagggggcagctggagcgggacgtgtcctgcacccctcgccctgcatacgcaggtcgggggcgagccgtgtgcgggagcgggtcagctgcggacgcggtgggatgcttctctaccctgcgctctccctgctcccaagactggccctcccgggggtcctctgctgcagcgcggcctcaggaccctgcctccgggtaccgccactcgggacgcgggagccctgccccggggagcagacgagctgggcggagctccagcgaggagctgtagggcaaagcctgacccggggactgcggtcacctgaacgaaacttgcaagagaaagcgctcctttcccggaatttttttttttttccttttttctcccccttttcccatttttgcaagcctccatcgaaaaatcctgtctcaggaaacgttcctttgggattcgcacttgaattagcagcaccccgaagagcttaaaataccgactttatctcggggaccacggtgccagcttgcgtggtgtgtttccgattcggttgaaagcgtatttccttttcggggagggagggtggcagcgccttgcaaggacgctccagctcgccgcttagtcacataccactgctcatttcagtattgtttgacaaaacagttttccataccgagcagaggggcgcccctcaagatcaagaaggtaggactagagctggaggtgtagatagcttttaattgaagtaattcatgggggtgcttttcttttttttaagtcccttagaagatttgcggagttttatcagcttgtaagtaccgaggtacgtggtggttttcgggagatcctggaacataggaaacgatgctttttagctttccttaacaccgggtgggggaggggggtaggagtgggggggggcctattttagttccactatggctctccctgtcttgcctggaactcaacaaattttgtctgccagctctgagaataaaggcttttgctaccatgcatggccctgggagcaggtttcagattaaatttctaccataaaacttccactccagaaatgttctaaggaactttgaaggattcacgcagtatcttttactatatcttttagagctccctatctgatagtaatctgatagattcagtagatgagcccttcctgtgttttagtgacttacatgcattcatggtttccgaaggagtctgggaaatggtttttgttttggaacaaattccaggcttctggatgtgctaacctttaagtatgagtcctgtgttacttaaagtcatatcgtgaatttcctgtttggtagaatcttctgggtaattgagattgtgcaattgtgtccagcaacagatcacattgcatttgctggctcaagctgcttaagctatgttgcagatgataccaacttggacatctttctcacgagtctacagaatggcagtttacactttaaaggaaatgtcaataatatctctcatgtatttgtgattgatctgttgcatcattgtttctctaatagttaccagctccttaagtatctccattcttcgttcctaattggtacagtaccaggcgtttgtttccatttgaatgctcagggagtgaagctcaggggcttctgggatggtgaatttgcaaggtttcctgtggacaccactgtcctggcagaagagccagcttcacagtgaagtgccacagtgaaaccaactccacttcctctagtgctgctgttttctgtgtaatataattgagcacaacctcacaattaaatagacttctaccaaacagcttcttaaacataggtagtgctggcccagtgatggaacgcagtgggacagccctggccttgcctgtgggaggccctgcattccagccccagcagcagtgcaccacacaaagcaggttgcttgaaataagggcccaaaaggtatgcagcaaaaatcctgtttctgctctcagccctgcatgctggtcttccagagacaaccactgtttagttccttgcctgacattccagagaggtgtagtttctagcgtagagatttactatgtagcctattgatgaggaaaagatagctgaaattgtgtgtgacccagcaaatgtcaccttcagttagatttgcaagtgttaacttctctcaagttatttgaaattgtgttttcaattagctttttaggtgtgactgaaaaatacttttttgtttgttttttgagatgagctgtcctagaattcacattgtagaccagactggcctccaactccgatcagctggactttgcctctcagagtgcctggattaaagacttgcaacactacacctgacttcttgaatttcttttttaatccactttgaggcctgtgttccctttcttcaccttcgttctctgagatagtttgatgttggggaataatgaagtccatttgaatggatataattgagggggatttaaactgagctcttgagaagagaggaggctgttctactggtgacagtggaataagtcaggagggacctgaagtctcttcaaaaaaggaatctctgaattggacatgatacactttgatttgtaacttaatttttttcttttaagtttaagattaattttaatttgtattatgcagtgagcgacagtaacaaaaaacaagcagcaaactgtagtattaccaactgtgactcaaagttgaatgagagtctagaaattgagaattggcttgtttttacaaatatctttctttttaaaagtgtaaagggaactaaactaagttctctaagttttaaggaactgtatttactgcctgaaaagggaaacaagcaaaatcattaagaccacattgttgcaaagcattgattgtgtggtttgggacataggggaaaatctttgtggtgaatttaaggaaatacaaacgggaacgtcagtgtctttcctctggggaaaggggctctccagccccttgctggacacacttgggtaccatgccttgggagtgagaattaagaattacttctagatcattcacagctatttattcattagtctggattacataacaccaaactctggctagaatcaaggctagattcttgagctaaaagtacagaacgagacagctcattttgtcattgggcttgtagaagaatggatggatttaatggctgcattttatgggttgcttgctgtgttgtaggcatggagatgcttagtttatgatcggcatgtagacatgtaacaaacttgtagctaaacatggtaatttcttctaactaaatctgatttagcatgctatataggacaagattattccagtaaatataacaaaaataaatgaagctttttttttttaagtattaatagccattcaccattagttttaattagccagggaaaaaaaataaaactccacaattaggtatgacttttcactcttgacgaccgagaaggctgggcctttactattagttgtaataatagccggtctctcttccttcccttcctgcctttcttcctactttccttcaggacacagccttctggtcttgaactcttgatccacttgtctgaggctctctaatgctagggtcataggagtgggccaccaagctgctttagctgacctatctcccatttactgttttgtgactaaaagtagactagttaactggatgttaccagtcttaataagcatttaggtttgaggccaacctagcctcactcagcagagtgaactcagtgccctttctcaagaaaaacaataaataaacaaaaagtgcccaccaaatatgattggcaagagggtgtgtgtgtgtgtgtgtgtgtgtgtgtgtgtgtgtatgtatgtgtgtgtgtgtgtgtatgtgtttagatttatttattgttatttgtatgaatgttttgtttgaatgtgtatttctacaccacaaagtgcttggtgcctctggagcccagaagggggtattggatcctctggacttggagttacagaaggttgtgagccaccagaagcccttgccagtgctcataaccactaagcatctcttccccagctcccggcaatggtttgtatggatggaggagagttcagctgagctagctgtagagtttattttgttgagttccgtttcccactcatgatgagcactacaccccaggcctctgagttctgccacttttcagttttgaatattgaggagtattcttttgatttctctttaattcagcctcagagatgatatgtgttggctttgccttctgtggacaatgttgggaattgtgctaaacccttaaagcttaggactcaaatgtcgaggagttccctggatgctgtcactttcgtattaactggcatcttaatgtgattctttggtttatcatgatcttcatttaaggcattatcagtccctctctttctagagtaggtgggttgcttttccttctctctctcttctgcatctttagtaacggaaccctgggggctggtgcacactaaccatgggatccagtgagctgcactccagcctccaggctggcgttttagcctagttctgtggatgagctggggttaacagaagaccctgtgctgtatgaccatcagctagagctgaatggatctgggagcaatcagaagggggagggtttgcagctgctaggaatcctacctggtcatatttgcctttgtaaagaccaacttctgtgctgaaaaagtaagccaaggtgggttgcagacagagcagagctggggtctagctccctagcttttcatttctgttccttaactgatcgaggcactaactaaactgtttgtggtttatgtaagcacaagagagataaatcatggcagggacttgacctatgccctgtactcttcatccacaagtccaaaagtgccagagccagaatttttgttgagctcggaaattcagggtactccacttcagctctctaaataacatgcttacttcaaaatttttttttgttttttttttttgtttgtttgtttgttgttgaagggttttgctgccaaaaggaagggatcaaagagattagctgctgagttcagctgaaaaagaaagaggagctgctggcaggctgagtgttttctaacatctttgacttccaggctttttcttaatacttgctcatattttaagtcacttgtatcttcctcagagtatccgagcatttaaaatcacatttatcttaatctttggttctgtgtctgttttccattcctgcctttccctcattgtgttacttggcactgatgtttttgtctgtaagactccaaggaatgttaacttgaatagatgtgaccaatacctgtctacagataccttgtaacagctctgctctgtcctgtcttggctgtcttcatgtgctgcacactctctggctctcttgctctcgctcgctcgctctctctctctctctctcgctcgctctatacacgcacacacacatacacacacacacacacatcccgcctacacacactgctttatcatctggttgcagaaagtaagttcctgaaaacaggttcccatggcaacataacagaaaggtaatagaacaaatgtttattctgtctagacctgttgtctgactaactgtagaccctgaacatgtttgctcctgtcacactgaacaatgtgattacagatttccttgtttccctcaaaacctcctccttgtagatcttagtggggttgccactgtgcttgtgacgtgaaggaagctgtgtgtgtgccctactgcaggtgtatactggttgtgtccccaagcttgcaggccacagcttacctaaaacagcagcagttgctcctttgttgtcataagttctttagcttagtattggtagtgttaagcgttgtttataaagtccaaagaaagcaagggacctggggaggtgccatagtgcttgccgtgaaagcatggggccgtgacttggatctagacagcatgtgaaagccggacacagcacatgtgtgagctcagtgctggggtgcagagacaggaggatgggtggagcctttggactagctggccagctagcctagccaagttagtgaggtccagattcagtgagaaaccctttctcaaaacacggtggaaggcacctaaggtcaacctctgacctacacacatgcacacacaaaagcaggaagagacctaggttgctttggaagtctttgtagcttttctttctttcatattgttcagtgtcatcatctcaaaactacctttatatttctaaagacagcccttcccctttcaagtgacattctagtagcttgtcaaatctagggcagcttgatgccagggtatgtattggaaatctgtgtcagagaaccatttccatctggtgattcagatctggaaaggatgacatcagcgtgtgcccacaagaggtcagaaaacaaaacaaaacctcttttgatgtgtgaaggctctgggaggctgttttagttaaatggggtgtgagagtaattggtcttgatcttgagccctttgctcggatccacttggaccattgtcatgctcataatcaggcctttgctctttcctgtaacacgcagcttggggtgtctggcttgtgcgagtactgagttcatcttgtattactgcattcctgtagcagatctaatttaattcccagaaataatttttttattcttagtatcttcagtaggtgactcctctttacccttcccccttaaacttcacttagggcttttgctagctctaacttccttgaacctaggtttagaaatacatcgtttaatctaccagttgccagtgaagactttagttctttctcatctgtaaaaaaagaaagaaaaatccaactccctagtaatcattgtgcaattctgatgttgacatcttgtgttgacatagttgtgattacctgattgtgtgtgtgtgtatgtgtgtgtgtgtgtgtgtgtgtaaatgttcttgcatgtgtgtttgcatatatgcaaagaccagaggttaacttcagttattgtcgactatcactttctactttattttttgatagtatctctcactggatctggagtgcatggattgcttagactagctggccagaggtcctcaggaatcttcctgtttccaggcacatcgtctcctacctgttttttgttgctgatgttgtttggtttatgttgatactgggatcaaactcagccttcatacttgctgctggagcacttttctgactgaactgtctccccagttctgcccttgtgtgatgctgcagtctggcttgcttaggtttctgattccttatgtcgtgttctgagtgccctatggttgggtgatctcttttgaaatgagcgactcaatgttaaatgatactgataccaggcaaaaggtttaaaaagtaacagtgttatctgagatgttggaaaaatagttgcctggttaagaacactggctgttcttccagaggatctgggttcaattcccaggacccacatggcagctcacaactgtctctaattgtagtcccaggggatccaacaccccacacagacttacatgcaatacacatgagagttttaaaaattgtgatctgtttttttttttttttttttttttttttttttttttttggtatctctgaacttctttacatccccaaacatggtgtaacatagggttcttggtttgtgcctgttctgagcagagggtgatactttgttgtgggtctgactgggtattatatatttaacagcagtgttgtcccctaatcttaagatttatgacaatttcccattgtgacagccagatttgtcttataagtggtgccttatgttgggggagggttggaaattagtcacctttaattgaaaacgttttacacaacagtacatatctagaataattggtcttgactaaaacctataggctaacttgaacactgtacctgtcagcagagaggaatgctacacatggggcttccttcagcatgaccgtcattatcttcaggagaacatgagaaagcattatcgaggtcccaagcttactgtggttagaaccgcttgcacagatcgtgtgttggccacgtgggtagctgtatagtgctgtgaagaaacaatgaatgtgaataactttgttttccttttgtcgtgattcctcttaggacttgtaaatgtttctggaaaggaaagcttattgagtatgttagcattaagtccaaatctgaaggtagtgcttttcccagcatctctggttttcttttcttgacttttttttttttttttttttttttttttggtttttcgagacagggtttctctgtgtagccctggctgtcctggaaacttactctgtagaacaggctgggctcgaactcagaaattcgcctgcctcagcctcccgagtgctgggattgaaggcgtgtgccatcatgcccggctatctctggttttctcgccctttttctcctactgtaatctttcagtaggtagctcttttcagagttcaaatatacagtgttcaggccagccaggactatatagtgagaccctatctcaaataaaataaaaatgttagaaaagcaagagaatttcatgtgaacacagaacaagaagtagcagtgttacccattgacgtgaccagcttatgcttgacacagtccagatatataagccatccctggcttggagcataccatgcacactagcctggctgcagatcatggtagtctccttctttctacctctgtctctgctgggattacaggtgtgcaccactgctcttattttgaaggttcttgttagcattttagtctttaaagtattagacagttacagagggtgagtttttagtaatctgtggattccagcagtaacaaaaaaattagctttattcttgactcagagccttccatttacactcatattttggtgtttcctgaattaaccatggtaatgcgtgaaaggttctggcatggtaataaataattctgtattagaatttgtaacttttaatatttaatgctgtgctttatatctttatttctatgtatatgtgtgcctgttcacaagtttgtgtggaagctagaggtgcagtctatcttccctcagacagagtgtcatcagcctggatctctagttaggctaactggccaacaagtcccaggaatcctcctcttctgtctctacacagtcctggagttacaggtacacaccaccacacccagcacttaagtgtggttctggggatagaacttgggttctgacagccccactttgtgtcgtcctcatacttcaaagtgctctgttgttctgatatcctgtgtttatctttgggcagttttactcatcatcgccatcatcattttgcatcatgactcatttttaagttttctttttctatatatatccattttgggcacattgccgtacaaaagtactcttggtggttggcactcctctgttgtgctaggagtggtggtacagaccagtaattccagtagtcaagaggttgaggcaggatgattttgagtttgaggtcagcgtggggcaccttgtgataatcttatatcaaaagaactagggacatctaatcttgtaaattccgagtccactgagatgtacaagattttgtagcaccctaccttccctgattagtttttatttttttaatgatttgtttgctttctgtgcaatggtgttctgtttgcatcatgtgtgtctttctttcttttcctttgctttgatgaagtaccctgacaaaagtaacgtgcaggaaaaaggtttctcttggctcacctctgaagaatacagtcctttatggtggtggtggtggtggtggtggtggtggtggtggtggtggggttaaggcaacaggagcctaaagcagctggtcccagccagtccacagtcaggaagcagagagtgatgaggtcatgctgctgttcagctccctgttcaggatcccagccagggaatgactgggtgttcccagcccagtcagtgtaataaagataatccctcacgcccagaggcctgtctcccaggtgagtctagatcctgctcagttaacagcactaaccattatagtgtgtgtgtttatacacacttaggtttaagttttcgatttactgtagtgaagaaaatttgttgatacctatcagacagtgttccagactcaccagcatgtggaacagactgcttagaaataaagaaactgattagggaactatattcacatagtgtggacataaacccagaagtggtggttgtctgtgcactatagtatttaggatggtttcatggaggaaaaaagtattgagagttattttgagagctgagtagactaatcagtacagcaggagagaatatcatgtaagtgaacagatgccaagaggaccagcctgaccccgctgcggctctcagctgtgtttcacagtcactcgggagtcaagatgtaggtctacaattggactcaggtaactgctttccctaatagttgacttgtgttcctgactagcagaacttctgtacaaagcagagagctgttatcttcacttatacagtggaagaaacagtatgcctttaatcccagcactcgggaggcagaggcaggcggatttctgagttgttccaggacagccagggctacacagagaaaccctgtctcaaaaaaccaaaaaaaaaaaaacaaaaacaaaaacaaaccaaaaaaccaaaaaacttcctgtttgaagtgttggcttggactgattggaggaagaaagggctgctcttgtacagtcggctgcagtttgtctgcaggcacccctgtctttttgactccccagccttctctattcttgaggtcagcagtgatctggttttggtatgtgtttgttatttttgttttatgatcaggtcatggctaggtgccaaggataaaagataaaccacagcttctgctctctcacagactggagtaggaaacaagtatgcagaggaaaggtggttctgagttccgtctttaaaggattagttacaatgtactgccaactgtagtagttctcaggttccatctaagggttgtagaatccatgtacacttctaacttgtagaaactacaggtatccatagattgacaaatcatggaggatttcaaggactctcagttacattttaataatcttctctttttactattacttcagagatatgctcaacttgcagtgtgagagaaagatctaaaaatactgagagtttactacatgggaagccccatgctagagactgcaggcacagacttattttatagttggatattcatttcaacttggagtatttaaaactaccttctggggctggagaggtggcttagttgttcagagtttgaactgttcttgcagaggatccaagttttattccagtagcttataactgcctgtgacctcagattcagacatggcctctacagacatggtactcacatgcacaagcccccacacagacacaaacaacagttttggggttttttgtttttttgttttttgtttggagaggaaaggatttattcagcttacacttccacattgctgttcatcaccaaaagaagtcaggacaggaactcacacagggtaggaacctggaggcagaagctgatgcagaggccatggactgatgctgcttactggcttgcttcccctggcttgctcagcttgctttcttttccttctttttttttatttttttatttttttaattaggtattttcttcatttacatttcaaatgctatcccaaaggtcccccataccctcctccccactttagtttttaatataaagcacattcaacatacttacttgagcctcttcacagcgttttaggctaggcggggctggtgattccttgttttattcctttggaaacagatgcgttaaagctttgtttgggatcatctcctagtaagtgccaaactagaactcatattttgactactggtttgttttttgtttgtttgtttgtgtgtttgttttttgtattgcatggggtacatttaaatttgctaattggaaatttctagatcaataaaagatttttttttttttaagaaaaagatttcttttatgtgcattgatgttttgcctgcatgtatgtctgtgtgaagatgtcagttcccctggaactgaagttatagacaattgtgagctaccatgtggctgctgagaatagaacccagatcctctggaatagcagccagtgctcttaactgctgagccatctctccagcccaataaaaaggtttttattattattattattattatttattttttgtatatgagtacactgtagctatcttcagacacaccagaagagggcattggatggctgtgagccaccacttggttgctgggaattgaactcaggacctcaggaagagcagtcagtactcttaaccgctgagccatctctccagcccaaaagatttttttaatgtagagaaatacattagaaataccagctttgggctggcaagatggcttagagggtaagagcactgactgctcttccaaaggtcttgagttcaattcccagcaaccacatgatggctcaactacccataatgagatttgatgccctcttctggtgcatctgaagacagctacagtgtacagtgtataataataaataaaccttaaaaaagaaaaaaaaaagaaaaagaaatactaactttgacttaacattggttatggaggtttgtttgtttgtttgtttgggtttttgttttgttgttttgtttttttttttttgcccaaaagaaatccaaaccaaaaaacccggtaagttggaatattaagtcatagtgcttctaacacatctatccaccccagtgtcctgccttagcaacagattacactgtagtgtgtgaacacccggcttcctgggacagatgctgttgctgagcattgggagagcatctgcctgcttatcatcagcctggagacgctcacgtcagaattccaagtaatttggtactgagtgcttgccatcttctcagtcttaaaggtgtatagaacaaagttggagagcaaattagagaacatctgtagtctgaataaagaattaagatgtcctgttgaggcagagcttagttccttcccctctactgaggactgtacttggtggctcagttttctactgtgtagaatgtggcaggaatgagtggctgcagaaggaatgctgtaaatgagattgtacctcctgtggtgctttttccctgggaacactgctgaaggagtaggatatccaacatcttgcttacaggctgctttgcaggccatgtgcatcaggatagctatgaattccacccaacacatgcatagatgacatcattctatcacagttcaggaggttggacacccctattctagagcatagtttctcagctctgatacactttggcatttggggtcaaatagttgttcataattcttggctgttgtatctcctgtaggatgcttagcaagcagcatctctggacaccacctacttggtgccaggaacattcttctctaagacttggttgtgacagtccagaatgacttttggatgtcctctagggaacaaaatcaccctttcttttcttttttaaaatagtatttatttcatgtatatgagtacactgaagctctcttcagacacactagaagagggtatcagatctcattatagatggctgtgagccaccatgtggttgctgggaattgaactcaggacctctggaagagcagtcagtgctcttaaccactgagccatctctccagccctaaatcaccctttctaaagaacttctaggggaagcctgcaaagcagccctgtggagagcccaagatggcaagcactacagctaagagaacccttggaaaaggatcctccagcctcagcaggtataaaagtgactgttgggctggagagatgttcagcagttaagagcactggttgcttttccagaggacctaagtttggttctccgggcccacatggtagctcacacccatttgtaactccaattccaaggcatgtgacttcctcttttgactactgcatgaacatggtacacagatatacatgtaggtaaaacacgccttcccatgatgaaaatgtaaaaacaattagaagagacttgctatggtccatctcttcactgcagctttcggggaaatagccagagccaccttgctaaatagcttcctagttcttgatccacaacaataggagataataaatcaggtttaaagctccacattttgggaatatttgttacccagcactagataataccctaagtgctatcataaaatgtccagatattgtataaggtagaaagttctgtacttcgggaagttagcttgtggagacatcagtccttgtgtttacccatagttaggtttgtagctcagatttatacagagatggagcctaggaactattgctatgaaagctttttccatgctggtagctagaccatagtaacttgagtgtaatatggttagtctgatattttagatttatttgaagtgagttaattatgattacttaaagaaatatttgctattgagattttgaaacagcaaaatggagaaatgtttctagtggtacactgtaatgaggctttggaaaatagccccttttataaaagaaggcacagtgggagggacataacactcaaaaatgcggtttatacctgtgtggaaatgaccttataaaacccctgattgtgctggagtggtaaaataagggcctttacatttgtctgaggctagaggatcctctaaaggcaccttacccgtgtggctattgacagaaggcctggttactatctagatctctgcacagggctgctacagtcttctctagatcagtggttttcaacagaggggacattttgttcccttggaaaccacattttgaaagggagggggaatctgggtgtcatggcatgcacttgtaatctgagcactggggcagcacagaagggcagatcttgggcttgcccagtccagcctacttggtaagttctaggccagtgagagaagagaccctttctcaaggtaccttttcaatttaaatgaactagacagtgccagaatagtagtatgtggcatagtcacttgttgagtggattgtagattcctttcctctgggctttcagtttgttttttagatggtttaaggcttttctaggagtgtctgcttttggaaatttataatttagagcagaatgcatgatttctgttctttccaaatactgctctgatagggagaggaagcagcatgttagtgcatagagctctagggcattagcagcctttgctggagggtttccaacccacaagaactatgtctgagtaactcctgcctgtcttgtacatgtaaggtgtattttcatgggagtgtgtgtcctctatggtaagaattagtatgttgtaatgagagcatgtgattagggaggaagcattagacggtcactgagctggtgctgcgttatatgtgcggacatctggtgtgactgcatcagtgagaatatcacaaaaggcagatttgaaaattacttggtaaaatgaaaaatgattgtgatttaggaatgtaggtttcagatttacatatggtgattatgtccgtgtaagtatcattagaaggaaatgtaattgctactaactagtagtttgcactaagttgaaaacaccactaagtgacagagcagcagtgtgagtcacgtatctcactctactaatggcctccatgagaaatgaaagcttgttttaagaaaaaaagagagagagaaaatggaaacatacactgttcaaaagagtgtaacaagtttaggatatattagatatttatgctgtatgaatacagtatttccctatgatagaatcattttctgcttctgaaacttgttcttttgccctcagtacatgaaagagtcttgctctttgctccatgtgtgtgaacggagaatgtactagaattttaaagtcctgttgttggaatttcaagtagtttctagttttcctgtgaccaggctgcaacagatatgtactttgaaccctattttgtttgtatctgtgccgatgttttgagatatgattttactctgtagtagactggcttctaccatgtcccatctgcctctgtcttccaagcagaaggattataggcatgaaccactcactactactcccagcttcagtgtgtgtgtgtgtgtgtgtgtgagagagagagagagagagagagagagagagagagaggaaggagatccttttttaaagatttatttatatgagtacactgtcactatcttcagacacaccagaagagagcatcagatcccattagagatggttgtgagccatgcagttgctgggaattgaactcaggacctctggaagaacaagaagtcaggaaaagaatgagagaatgagaatgaataaatgaatgagtatgcatcattacagacttagacttgtggatgtcggaggacaacttgcaggagtcagttctctctccttccactgtgtaggtttccggggctgtattctcaggcctggcagtaagtgcctttaccctctcatcttttctttacctgctgaaccatttcacaggcctcggtatatatggttttttaatccaggatttggagaaatgtttaactgtctgttggctgtttaattggtcattcctggtatcttgtgcattttgttatcccaggtagatagtaagttttgtgacatcagctaccatatttactgttttcttttgtattcataaaataaaataacatcgcttggtggtaggggcactcacctttaatccaaggactggagaggcagaggcaggcggatctgtgtgtccaaggttagcctggtctgcagagtgagttccaggatagccagggctacacagagaaaccctgtcccccaaaaacaaaaccacaaaaaaagaaaaaagaaagaagccctataaaccaggagtggtggcatgtgtctctaatcccaacacacagtaactgacaagaaggtcatacattcaagccagtgtagaaaatttagccagacactgtctcaaaataaaacaaacattagtaaagggtatgcggctcagcttactgaccagtatttgtctggtgtgtgtccaaccctgagggaactgggaagtgctaattgaggaaactggggttttgagccctttgtatcacgatggtgagggaaacagatggacttcttgtcctcaacttgcccagggcaggtggcagtaggagtcattattaatgtctgataattgagttttacaaaatttgctaaacctgccataaaaactagtaccttaggttagatacagacattatatttgttttatatatgaggttggtagaatttaattaacttcctcaagagttattaagtggcaaagctagtattaaaaccctgatagtcctcttccagagcccagcccttgtttaactacctggtgggtggtaaataaacatatactaatcattgtgattattggggactgtagggtccctgtgcggcatctgtcctccactgagtggctagtgaagtcagagggtggaacctgcccctcttgctctggagctggctgcaaaagagcctgggtagtgatcataatgggcagctgttctgctatccagctgagctgaacttgtaggtgtggccacatcctgctggccctccctgctccccacaggttcatccccaaggagaggacagagttacaaaggctgcatgaggcatgacactcagagtggaagttgaagaagccacattgtgtgggggccatggccatttcctcagtttctaaccacacactggtaaagggaaggttgtcttatggtcttatggagataaagggtgaaagacggagagtgcttgcctcaggtgggtaggaacccctgtggaggggtgactctgcctatatatacttgaagaataaagatttagttgtggggtgggtgcgggtaggcgagaaggaactagcagcagcagatccaaatagtttacagaactgaaagaagccagcacaccagagcaggcctgtggtgggggactactgattgggtgagccctgggattgggtcaactcatctgaaacttagacagcggtaggaaatgtgcatttttattctgtattacaagaaaattccttgagtttaaaatagtcatttattattttgaaattgcacgtcggagggcagcttgtaggacggatcttttcaccttgtcaagttcttaagaattaggtatcttctggaccgcccacagtcctgctgcccagaaaagagatttcaagaaaggataggctttgggggttttgtgtttatagatggaatttaacccgaaatgaccagttgtgtagttttctcatccctgcaaccaaacacctgagaggagaaaggagccagggcttacggtctccatttcacagggctcccccgtgcttgcctccatccataccctcgagcagagcacgtgggagggcataccgtgggctgttctgtacacctgagctggcaggaatgggacagaagagagggtgcccaaccaaactatctccttccagctaagctctaccaccatagtgaaaacactgaagcttttctcttttgtttagtatgaggtgatgtactgtgtgttcttgtgtcgtggtgtgcgtgtataggacagaggacattttcaagttggttttcgccttttaccatttgagttcctggaatcaaacttaggttgtcaggcttgtaccctcatccactgagctatctgcttgggccctcccacctccaaagattttagaattctctcaatctggaaatggcacatctgttgttccagtgttcagaaggtggaggcagaaggatcaggaatttaggattatcttcagcttgagagtgagtgtaaggccaacctggatttacctaacagcaaagtggtgctaccagctgggaaccaagtgtttaacccatgcgcctgtggaggatagttcacgctgaactgtaacacagctgtaaaggaggttataaagatgagaagatgaaagccgcccaggaccctcccctgaagaactccatcatcaggagcttatgtagtacaagtatgtgatgcagagaaagtcccagaaagccccatgtccattgtaggattgacaggaggaagggggagtctgaccatgccaaatgctaggccagaacactggccgtctgacgtgcttaccgagcccgagcccggtagtgggcttgagaagagcagctccagcaggctggattgaaggatgaatggagctgagaagagcattgtctgtctagctgtgcttctcaaacttaggaactgtgtgaatcacattgtcacaatgcagactctgctctgtgggtcaagatcctgccttctcagggagctcccaggcagagccaggaccactggtctacagagtgcactttgaacagcataggtgtaggttaacagctcttgttttagttagtgcatgaaggctttgagaattgcgctctcttcagtttctgatttgtagctctaggatggtgtgtccaggagtttccttactaattaatccttgggtgaagctgacgtgaatggttcccataccttgagaatttggagattattaagttttggagaattttttaaactgtgaggaagatcatcaaaactagttaatgaatatggaaactttctagagcacattctgagacaagtgagacatcagtgtgtaaataactcagtggtagagcactcgcctacccagggttcagttccgtgtgtgtgtgtgtgtgtgtgtgtgtgtgtgtgtgtgtgtgcgtgcgcgcacgcgcgctcgatgtgtgtccatgtgtatgtcccacgtccccctacgtccccctcctcccctttgtgagtcagcaacacgcaggaggaatgttgttgagaagatgtgggacggctccaaagcacagtcttgggtttgatcagagggcatgtgttccttagagtaagagagactatggagagtgttggctgagggcatctggggtgcacagatccacagaagagatggagctatatggatctccctttgctagcttctgttctctcaaggaaagggagggagagaagggattggaagtttgagaggtggaaaagtcatgagatcttttttcagagtagagaatgaccttgtgagtggagtcactgtttcagtgtttgcaattatgttcagaagtaaaaacttagaagcagtctgattgtactttttttttttttttcaaccgtagctagccttgggtaattaggtggttgtggtcacctaaggttagacttgatccaaggagagggtacaagggagcagttaggaggttgaagacaggagagctggagacacaattgatgaggtcagcagcctcgatgcactcaaggtagagtagtgaaagcaagccgaacggttggaggggcagtcggtgctggagagagatggctgaaatatggccctgcatgaggtggagtttccggagaggtcaggaaacgggatgttggatatgccctccagctggatgatatagttgccaagaatgttatcaaaagttgggagtggaggggtacttttctgataggacagacttttggagggtgatggaaaattctagtccatagataatagcaaccctgaagaagggttgctagcaagggcttttagaaaacaaatagaaagtgatgtcttgaagtgccgttaccgcgaagtgcctgttctgtgagagaaatgtctgtggtatagcctcagaaaggaggctgggaaattccaagagtgttaccacaaatcaaattgatggcgaggatatgtgtaggtttgggggtaaaaggaggactcctgagcaggacagactggtgtgcaattgttctgcactagccactgcgatgctgcactggcgtcatgttgctgctcatcccacctgtgttcattcagtggtcactgtgtgcatcactgttgtgagtgcttattctcctctgtcactcttcccacacttgaggacttcctactgtttgtgcttcagtctctgggaattgtccagcacttggaaggctattctcctggaagcaccgtaatttctggggcctttactttgaccagcacatagtctttaagattatcagtgtgttggctccttggacaggctgctgcctggtttcatgaaatttactagaaaacagccatacccattttataggttcctattatttggctgctttcaggcaacaactacaagctgaggagttgtgtcagggaccttacctgggctttggagagtaaaatgctactgtctgcgcctctctggaaaatgcttaccaactgctgatatagacaaaagaggcagaaagaagcatctcaaataaataggtgccttctctgggtggtgggtgatgagtcattatagtctttctttttgtgtcttgccacaaatttaatttggaaatgaaatagcctgaaaagtactaaaaatagtatttggaacactctttttttttgtttttgtttttgttttttttgtttgtttttccagacagggtttctctgtatagccctagctgtcctggaactcactttgtagaccaggctggccttgaactcagaaatttgcctgcctctgcctcccaggaggcagaggattaaaggcatgtgccaccatgcccggcttaaaatagtatttggaacactcttaatggtgggttctgttgttttccttacatcagtgctgcttttgatacaaagacggggcatcgtgtggcagttaagaagctgtcgagaccgtttcagtccatcattcacgccaaaaggacctaccgagagttgcgtctgctgaagcacatgaaacacgaaaatgtgagtaattccttttcctgcacacactgaaggctttgtatgcaatcctgccggtggatgcatgtcacactgtcagagttgggttcatgccgcatgagggctgcagctttggtgccatggggctgtcagatcatcgcgaggatgaccgagtgcctcttcccagagttgtacttttactacaggatctcaagaagcatttaattttctatttttatttagttagtttctgtgtatgtttgcctgctggggtttatgtgaatcatgagatgcagtacccttggagaccagaagatgtcagatctttggaactggactcacagatgcttgtgagccccactgtgggtgctgagaaccaaacctggaggctctgcaagagtaacccccactctttgcttagatattagtgtttggtgtgttttgtttgttttgtttttttctagacacagggtttttctctgtatagccctggctgtcctgcaactcactttgtagaccaggctggcatcgaactcatagagacccacctgcctctgaacttcaagtgtttgaattaaaggtgtgagccaccacaactggctgtgatttgtatttttaacatgtatgtttgtttgtttgtttgtttgtttgtttgtttaagacagggtcccagtatgtagccctgattgggttggaattacggagccactctccagccctgtattgctttgtatttttgaacaaagcaggcacggggggaatgagaaaagatcagaatacttacacttaatgtagaaattatctcacctttcagtctatcaatattatgcagtgaagaacaaagacttgggactgaagagatgtctcagtgcctatgcgcatttgctgctcttgtagaggacccaagattggttcccagcacctacattgggtgattcacacctgcctgtaactctggcttcatgggttcagtgccttcttcctcttcatccagggatacacacacacacacacacacacacacacacgcactcacgcacacacatgcaggcacataggggcaggggagagaaagacagacacacacagagaaagagacacaaaataaatattacataaggcaggaatcccaattcttttttctgtctgatacactactggctatgttaacaaattctaaaaagatagacggttttttcatggtattatgtgacgtgataagcaggtgttgtggcacacatctgtatgaacttgggaggtggcagcaggaagatcaggagtccaagttagcctgggccatatgaaatgctatctgcaaacacccaaaccccagaaagaaatgatgttagcaaatataagacagaaacacagtctgggtagaaaatcaggacactgagcacatgggtgtggctgcacttggagttagttctaactaaaacactggagtggtactgtaagaagaccactactttcccactgtttgatttttatgtgttcttgcaggtgattggtctgttggatgtgttcacacccgcaaggtcactggaggaattcaatgacgtgtgagtgaatcttctgtgtttgcaggctcagcaagaggatggggatgagcagcccatggaactgctcagcagtatccaacctcagaagattattctccacctattctggtttcttgggggagggatacataaagtgaatattatgtatggtgcattaggacacagtgtggcccattcagagccaatgatgagtccttacaaaagttacacagagagggagtatagggaattttggttataattgtttttccataaatgcaagtccacataggtgtttccttctttgatccctcctgatgccaggtgatactgaggctacatactgatacttaatgacatcttccagtctgatgccttctagaaagcagagcctggaagtcccaggaaccacaagtagaggccaggagaaataaaacagggacaaaggggatggtaatgcatggatgtgttagtgagagcacctggctgggaactgccagggtgtaggagccctacagaatgcacctcggatggggctgttcttctgattgcaactaaggtttgctccattgttcatgaatgctttgcatgctgacacacatgtgatgcaagacagcaggtgtcttgtgcaaatccagccttctcaccaccatagctttcctagtatgattaacacccgtattaattatcatcttgacagaagccagaatcacctgatgggcctctgggcatgcggggggggggggggggggcggttgtcttgactattaattgacaagggaagagccatctaaactgtggttgtgactgttccctaggcagggcatctgcagttgtgtaagacagaaaaggcagcagagcagtagcatctagttcatcttgcttgctggtgcagtgtgatgactgtgtacagctgtttcccttaaaatgcttatgtcagcttgtttatcaaaagaagaggaaaagacacagccccaccccacctaagtaagagacacactgcgctgcagaaggggaaacatcttaagcacagctgtacgtttggtcttgttaggattctcgaattcctaagtgcaaaggatgtttattgtaaaagctaaactgtaggtcatcactgctcttccctaacttcttgtattactaagccacctttctctacgacatgaccaaagaaactagcttatgtaaagagaaaaatgggttccttacctcctggttttaaatgtttcacatcatagtcagatgagcccatggcttctaggcttctggtagaggtgccaggtagtggtagagagtgttggtataacagaaaccactcatctcatggctaagaagcaaaagaaaggacggaccaggatttcacagtctgcagtcacacccctgatgacttaagccttgccatgttgtgggaatttggaattttggtttcctcaagaaatgctgtgcttcacagaagtagtgtatgactgtttttgttttagtcccgggtatggggatatggtgctgccgtgcagcagctgactgtgatttgccttgtcctctagcacagtttttttccagctatagatagtttctgcaattgtgtaatgtttggaattctggaaactttttacaaagtatgtaagtgctagggcctcaatgctaggttgatggtgagtggttgtttaggagggctggttgcagttgctagtggcagtgctcaagaaaaaccagaagaaagaaattagattcagggatctctctctctccctcccaccttccctctctctatctagtgatgggggtgagcccaggggataaagggtgggaaaaagaacccacaaagtagcaaagaccggctacattacaaagaccggctacattattgcccagaggtttggctgcttttgtagcacccttggaaaggaagccattgacatggggttgtgtggggttattgctcaccatccacactataccaacctcactcctgatgctcccagtgcaaaacctgcttctctccattctttctttggaatgtatgcccacaccatttgtcttttccaacagaacaagcagacagatacatttgctctcttgtgtcatgtctatagaaaagggaaaaacaagtgaatgccaaagatggagcaggagaggagaggtggcagcgggagggaaggcagcttggcccctgtacaggagctcaaccacattagctcacctgcaaatgcaagtttaggaagcaagtgaaaggaagactggcagtgagtaagaacagtgtccgcctgtcctgcagtcctgtctcagcagcagaacagaggctccacccacacaggctgagcagactacagagccctgctgcagccggtgcttggaaaagagaatgtggtggagtctaaatgagaaacaaatgctgctgcagtcttttgtgaattactttgaaaaacattggagttaggtctgagtgttaacctaagttttagtgagggtagactctaacagaaaaaaaaaaagatttgtggactccaagtattgggttttgctttgattgagcattctggaatcaaaagtggacctatttttgctacattaaaagggcactaatgaaggctgatgtctatttgaatggttaaattttattgtctaggtttaacccacccactaccagcatttaccccagtaaataaagttgtattcatttattacaactttaaagttagtcttttcaacaagagcagaggcttctgtttttacctctagctggtttttaacaactttttaaaagatttatttttttattgtgtatatctgtctgtacacatatgtatgtatgtatgtatgtatctggcatgtacatacctgatgtctgtgtaggtcaggaaagtatgtcatatcttctgctagagttataggtggttgtgagccacagtgtggagtcctgtgcaagagtagccagtgctcctaacccctgagccctccccagtcctagctcttgtttgagcaaagcagctctcaagtgctgtctggtaggtactgtgccctaagtgctgagtacctgcaagtcagttaacatgctttccatgagaacagtggagagcctgtttcagtagggagtgtttcattgctgactgtttgtgatgttgataccagtgaaaaggttagcacccttccagaacagagcttatctctgtgttctccacatactatacgtcttcacacaggtaacaagaatagtgagtgtagtgttgccgattgagcctcgaaaccatttgtgttcactaagaaaaaattgtcgtgcaaacagcatactaaggattagagactaaggctgccagatgcatagcaggctccaggtctgcatctgcacttcccatgctagtgagcaaggtgtgccactggctttcctgtgctttcccgagcctgtgcgtttatgtttcagttactgcttttcctttcatcagatggctcacatttttgcttcatctgaaagaccacgcgtgtctttaagaaaggagcaatggactggcagtggactgtttttgcttgcaagtcatggaggtggcttgggaagtgagcacatggaattctgactgctgtataacagcagttgcaggacaggttgcacattacagagacacaaatgggagatcatttgagcacttctcctgttgttcttagttcttgcctgtactggtcagtgtacaggtatcattgtgtatcctgaatatttacaaatgcagaaagtttcaaaagactatattacttttttttttttttttttttttttggttttttctgagacagggtttctctgtgtagccctggctgtcctggaactcactctgtagaccaggctggcctcaaactcagaaatctacctgcctctgcctcccaagtgctggcattaaaggcgtgcaccaccactgcctcacagtctcccaaacattcttaaccaagctgtcttagctgagtaggcagggggtacttttaggactttttggaaatactttcttcaactttcaggaaagccagcttatatcagagtaagtggaaatgggacttccttcccaacttacaacttttcctccttgatttttttttcccttccttttttccttttctttctttgtttccttcactcatttatttgagacaggatctctctgtatccctggctgacctgggattcccagagatctgccagcctctgcatctctgcttcattcactctttatatttgttagtttatctttgtgatggaatctcctgcatcccaggatggcttcaaaatgactgtgtaaccaaaaatagccttgaactccagatcctcatgcattttcctacctagtttatgcagtgccgggaatagagccctgggctgcatgccgctgagcaagcactctacctccaccagcttagccatgctcattcctctgctcagtcctgaagagacaggaggagtctcgaagtctgctgtgatctagcggatgcactgcggggcacttagcagctctcattttaataaagctggagagcagttggtcaccaagctggccttgctcctgtgcagagtaagcttgggcactgtggttcagttaactagaactgagggcatcgagtcagcgaatacattctgccaaacacggtcctcatccttgaggaagacagcaaacatgttctctaatcattgagtttaaatggccacatagtgtgcagtgtggtaggtttgggagacccagcatggtctggctctgtgcactctgaccttgtgtaacccactttccacttgtgtctctctgtctttgtattctttctaatacagacaaccagccacatcccagcaaggcctctgcctgctctcctcacagcctggcctctccacacttgctgattggttcccctcagtcccatacaaggttctctggatcagaacctttattcctctcttgtgccctagctgggctgtgatctccaggcactatctgtgccttgctctgcctggtactttgcaaatattgtcaccatatatttctgtctgttttctcccttgccttgtaacctctgttagagtagggtgctttcctttattcctaattgttagtgtttggagcagtagcaggcctacctgcagatattatttgtagaagaaatgagtgcaagaactaacaagttctaggacagccagggttacacagagagagagaccttgtctttaaaacacagaacagtcaatcaaaaagcatgtcccactcaagtggctagaactgtaaaacatgcagatggtctgtgctatcagctgggttgttagcagcatctcaaacttacagaggacttgagtgtcagtcttgtcaactgacacaacactgtgatgtagacttagcatctcaggttgtcagatgagggaacagatcgaaacctttattgaataagaatgtgaacaaaactgcttccttcttggtactctggctccatttcctgttacatatccaaggccatacctgcaccactgtgttctattgtatttaaagcccagtgtgcaactgagagccaataaggaagtagccagtaggtcagaggtcactggggaaagtaaggggctggaaggattctctctcactgtgaatgagggagaagtgtgatcattggttaattgtcagctcctgccgctgttttaagccctgtttaaatctggaagacatgctctgccttttcttaattacttttggatagagcccatagtggtggagatgccaagctataacaagaggtttttgttttgttttgaaccttctttgtattctgattttaaaaagcctgggatggtgggaatcttaacagttggttgtcacccaagtgtggctctggagcagggcagtttggagctctgaaaatcgtcaatcctgagtcctacttagtgctggttaagagacaggttatttgtctcacttaagtttagaggatgccacatcccaagtgagaagcaaactggaatccagtcgggaacgtcgtcgttttggccttgagcttacaatataatctcacttaatcttttaaagatgaggtattggatagctttcccactgcagtgcactccagccttccttaaattattttaagaatttactatttagtccagtgataatagcaagcatgtcttctcaaatgccattaagtacactcaccaggatcagatactgaagagacagacaagtggctctctagtggtgcccttttcagattatccattctgcttttatggtactggggtagaacatgtgaggtcatctgcgttgtgcttctgtatcctgctgatttgagaagtggcttgcctcccatagacagtaactgtggtgcttgctcctgtcctatgatgtatgacttgcttttgaactcctcctatatatgagctgaaatttgatactttggtaatagactgatcttggatcttgtgggaccagcatggaattattagctgaaagaactccaccataaatactgctttcggtcaactagtagcccaggaggggagtggtgtctatttctgtgcaagtgactaattgtacgtacacgatggtttgaccgtctcatcctgctcttacaaatgtctgtgataaaatggatcaggagtgcaggatccaccacttatgtctcctagctcatcagagacagttgtgtgtgataggaagggggctgggagtgagttacataggggctgtggagtctaaccatttgctgggcctgtgtgtttgtgtgatggttgcagatgcgtgtgcactgtggtatggaggtcagaggacaactttaggaattgagctcaggctttcatggtgagtgttaccacccacagagttcatcttagtagccctgctctgcattcctacatttatcagattatatgccactttgaagctaaaaatgcacagtcctgaagcaggtgttgggttggctgcctctaagcccagcactcaggagggaggcaaaagaatgtcaagggcagctcaggtaaatagtgagaccctgtctcactagctccaggcatgcgggacctcagttgtaagagttgctgcctcacatgcacaagacactggttcctcagcaccaccaaaaaacaaatgactgggataaggaaagacagtcttacttcattcaattttgaggaactgttagggaaattgtccaactatggtgacatctgtaaaatgttaaacttgtatgagtttatgtatgcatgccacagcatgcacgtggaggccagagaacaccctacaggagccatttctctccttccatagtgtgggtcctagcatgggcctcaggccattaggcttggcagctgcctttgattatgttctttgggggcttggaacaccactcaaatcaaaactactttgataaaaacacttcaaagagtctagtttactctttgttttgttttttgttttgggggtttctctttgtatccctggctgtcctggaaccgctctgtagaccaggctggcctcaaactcacaagagatctgcctgcctctgcctcctgagagctgggtttaaaggagtgcaccaccactgcatggctactctttatttttctaaagaaggttttgtgtgtatgatagagtatgtgtctgtatgcacatgagtgtagtgcccacagaggccagaaaagagcattaggtcttctggagcaggcagctctgagctgcccaacatggtttctgggaactgaactcaagtcccctcaacagcaggaagcactttcactgataagccatttctccagcctcttaatttactcttaaattatgtgtgaagttcaccttttccttaagacaaaatttaggaaaatggctaaaatctcatttttaaagtcaaaactaggccaagtccctctaatcttcatatagcagtcagtcctcctgagcctcgtgaggagaggacccctgcctgccctcacttacacatgcagccctggtgctctgctatgctctgtcccatgctgctgctgcagtagtagtgagagtttgtgtggcttgagtttgagagcctgttgtcctgagtgtactcataccctcatctcactccataaagcaggacggacactcagcatcacacctgcgggagctctggactgccttgctcctccttcctttaagggagagcaggatagtactcataagccttttgctagtcatttttgaaaggattccactttgaaaatcttaaaacagcctggggtttaatgaggtaaacataggatgatgggattgcagatacgtgatcctttattatgtgagtacagttaatcatactgcaaaaatgagacaattgaagggcagggggtgtggcttagaggcctgaagcatcaggtttgatctcaagcactgttgtcttgctgtgaaaaaacaacttagagaggagagcatttaatttggggcttggttacagctttagaggtaggatgctttcatggggtgcaggggctgggcctggttggggttttgaaacctcacagtttatcccaacgaccccctccaatcttagttatggttaccattgctgtgatgacatgccatgacccaaaagtaagttgggaaggaaaggatttcacttataatttcatatataaagcactgagggcagcgacttggaagccatggaggaatgctgctgcttactggcttgctcttcatggtttgtttagcctgctttcttacagaacccagaactaccactccaggggagcatgacccctgtgggctgggctctccccgatcaatcactaattaagaaaatgccttacatctagatcatgtggaggcattttctcaactgaggctccctcctctctgatgactcttgcttgtgtcaaattgacacaaaagtagctaatacacctactactccttctcaaacagttccataactgaggaccaaacaaatctatgaaccatgggggccattctcattcagaccaccacagtggtgcatagtccatcatactagcactcaggaagtagaggctggaatcagaaagccaaggtcatctttgactaagtttgaggtgacctgggccacataagacttgccttagaaaagagaagacagagagaaagaaggcacttacagtgcacgtgctttctgatagcgtccatccaggtgtctcttcacctccgtgctttctttacagatcaggaaaaggacatagcaagtaggagtggtataaatgtggagaggatagtgcatgtaggaaggcaaggctggtgtggtacttcttaggcagtctttcctgcatcggtatataacatacatacacacacacacacacacaccccttgtgtttccaggtacctggtgacccatctcatgggggcggacctgaacaacatcgtgaagtgccagaagctgaccgacgaccacgttcagtttctcatctaccagatcctccgagggctgaaggtacaggcagtagctttgtcatttggggaagtgagttatctctcccgtgtagtaagaacttagaaaagacagtttttcttatgaaaaacttttaaaagagttacagaatcaaccaaccctgctgctcttgtatttcccaggccagtttactagaaagcaaatattgtccttccctttgatcctttagtctgaatatccaagagacttttagccacattgaaaagttagtagcatcctgatacaacctggtatccagtcagtgtgatactgaggctttaaaatattcttcggggctatgagctgcttttatattccttctcaggacttgagaggtagaaaaaagaaggaccactgtgtgcagcctgagtgccagccaccaaggctgtctggggcggggggacctcttcagaaccacctttgatgtttagcacattggagcccttactgaagtcatcctctagaaagaaaataggctgacaactccagtaggtggagtgggaattagagtttgtgtaatggcccgctgtgagcgcgcacagtgcttgactgaggggagagcactgcaagcactctagaggatatgacaccaaaaagctgaagatggctttcattcaacctactttcttctcttttgcagtatatacattcggctgacataattcacagggtatgtattatacctgacgtggtaattattttggtagtgtgggtggggctagaagagtgacatctagttcagatctcagccttgtaactgaaatagtgattttctgcaaccttgttctcagtgtagacctggtaaaggaacacttgccatttgcctgctcaaggcttggagtctgcctttctctctgagagctcttccacatgtgtgcatctttaggacctgtagggagcagactctgtgcattgttgataagctctctctgcttgctttccaggacctaaagcccagcaacctagctgtgaacgaagactgtgagctcaaggtaagaaagaagagatggactgtttgcttctcttaagtgttccccagaatcgtgaccaaccacacttaagccacgacttgctatctaaaaagtcatttgtccttaggaatatcagactgtgagacttgaactaggctgaacaagctgtttaagtaactttggaagtagatgtcggtatcttgttttcatttttaaaagataattaaattagaaatgcaacacattaattgtaataaagggatattatagtctaaatgtcagttaagccttcctggaacactgataattagataggttcctaaaaggaaaaattgcagatctcagaatgatctaaataaaacctaagtaaattaaaattaagtctttaaaaagtaagtagaggctgccccctgcccagactgacgagtcagccctgctgtcccgagcgccatgttgggttcttctagcgttgccgccatgaagaaggtggttcagcagctccggctggaggccgggctgaaccgcgtgaaggtttccctggcagctgcaggcttgaagcagttctgtctgcagaatgctcaacatgaccctctgctgactggagggtcttcaagtacaaatcccttcagaccccagaaagtctgctcctttttgtagtcatctatcttgaggtttctcaaaccacttttcatgaaccagtgactattcaagagagctaagtttgaagtctgtacaaaagcttctctttaacacgtgccataatacactatcttctgctcgtcagtccttaacatctacctctctgaattccatgggtttctgtctcacaaggtttaactattttatatacactggctgtagcatacaataaagcagcatacaaaaaaaaaaaaaaaaagtaagtagctgcccaaggagctaaagaggtcagcaaccctataagaggaacaacaatatgtactaaccagtaccccccagagcttgtgtcgctagctgcatatgtagcagaggatggcctagtcagccatcagtgggaggagaggcccctggtctttcgaagattctatgccccagtacaggggaatgccagggccaggaagcgggagtgggtgggttgtggagcagggcagggggaggatatagggaactttcgggatagcatttgaaatgtaaatgaagaaaatatctaataaaaaagaaaaagaaaaaaaggaagtagcactatgcttttttatgattttatttatttatttatttatttatttatttatttatttatttatttatttatttattttggatttgggtttttcaaggcagcagttctctgtgtatccctgactgtcctagaactcactctgtagaccaggctggcctcagaaatccacctgcctctgcctcccaagtgctgggattaaaggcgtgcgccaccactgcccagcaatgaattttttgaaatgtctgcagggggctagagagatggctcatttcccagcagctacatggctgctcacagccatctgcatctagagcatctgatgcccactcttcgtctctgtggacactgcacagacttggtgcacagatatccatgcaggcaaaacacccatgtacatgaaatggaaaaggaaaagaaatgtacttctttgtagagtgttgcggccgccagcagctcgcaacatgaacggttcgactgagaaggctactcgagctgtaagagaggaatctagacggggcgaaagaagaaacggagccaagataaattcattctgatcaaagctcaaattttattgttgggacactagttataaaagaagggggaggggacccgatttccgccaaataatctctggtccagtaaaaaggtgcacgtgtgtggctccgcaggttctagcagtgggcgtgacagaacagatgagcgggaagctccacccctgagcaagcaggtttcaggctgggggaggagagactacagtagagttgtaatgtccatgtaaccacagtgatgacatagtaacctccatagtttcaaaaggtttgagtgtcccctcctagtgacccatgactctcacccttcattcaatgcaggcagccactgacccaattctgactctggattaatcttgtttcgtataaatggaactgtcgagggtcatgcacattactgaattgtgtgctcatctgtattgtgtgctccagcattcccagttgttcagcagtttccttttcataagcacaccaaagtttgtacgctgctgtcctattagtgagcacttgtggttcggaatataaacctattgtgaacattcctgtgcacacccgtgtgggcatatgtttttatccttgagtaaatgggggggggggttgctttgcttgaagctttatttttaaagtttcttggagtgttttatcatgtcatgcattctaagtgaaaactggggtgccctctttctgaaagtgacttttctgctttcctggttgtgaggtagcagaacttgctgctcccatgcctgacttctgctcttgcccttagattctggattttgggctggctcggcacactgatgatgagatgacaggctacgtggctaccaggtggtaccgagccccagagatcatgctgaattggatgcactataaccagacaggtactgactgcttcagtcgcctccccagatggggaaggggtgctccctaacatggctttcaggtgacttagcagctggggccttacttcttacgtcttccgtgtcattctttctgtgacactacctgaggggagtggtctcttaatcctaacacctatgtttcctgggaggattcaccatttaccagtaacaaagttttgcccctttgtgagttgcttatttcttttatcttaattatattctgagttgcacgacctgtggagtcttttcttgagtgacaattttaataagtttgtttctatataaaaaagattgtcaaaccttgggtgtcttaaatcctgacttgaatctctcactcatagttagagtcatttgtgtccatgaatatgaatagaaaccatgtcggtgcttgtatcctctttactgttccagtggatatttggtccgtgggctgcatcatggctgagctgttgaccggaagaacgttgtttcctggtacagaccgtatcctttaacacgttttggccctgtttctcatgtgtgtgctgtggtagatgtgtgcttccctaactcattggtggggaagactcagttctttgcagttccagcgtggcttttgaggtctgtcctcttgttgagttgctcaccttgccaagaaaaggctttggggttgagaaaacaagcttagaactcacctgtcatatttttgcagcaagtccctttattaatgagtttagtctattcctgaaaatccttggtgagtgtacacggccacctgtattcagaggtatctggttctcacatttgcttatgtactaaacagactgtgggtccctctgctgacacgcccgctgagtcctgtcctatttgctgcttaacaaccaacaaaagaagcaggaagagagaggccctgctgtgtgctcttgttctctacaccttgttgtccctgtttagagccagccccactttagtcccatacttagcaccatctgggttttgggcactaagcactcgttcctatctgttgggcacttgtcccatggcttaggaataatatcatttaatgtgattgtgagaacgcccaccagctctgcttaaggagggtgttaggtgaaaatgttaaaacggatgcttaaaatccatcttcaggaggagttttgtaaaactctagtagcctgcaactggttttggttgggcttttagaaataccatagcgacacatgcttcaccttctaactgcagggtgagctcttgcctaacacattgagactgtaagctcagttgctgtccacttaggctgcccttctccagggactgccatgccactgtgtctgtcgtgacagttttctgctagacaatcttttatggagtggaatcctctgctccccggataatgctgtctgctcctgtgcgtgctgcttggtgcagctcactctgttattctccactccaggatgtcatcacctgaccatctctattcccttgagattttggcttgcttacagtccgagctcagagggttaggcagggtttatgatgcaccttgtgctgtactctgggaccatcctgaagctgatctctcttctcttccttttcccctgctgtctcttcagctgtcctttggctagtcccagagacacatgccttcataggcagacagactaacagaactatccttagggaagtgtctcagttcattgcttttgtagtccatgtatgcacagttagatgctaaggccttggaatttcgattttagattttagctttttttacttttgaactatgtggctttgatcaagttttctaatatccacatctctctatttccttaactgtatacagctcagtgattgtgactttactgagtccagagccatcaggaagtgctagcttcaccagtggctgtatcagtagcttgtcatggccacagacagatagatacctgagagaaatacttattttgggctcatgggttcagaagtctcggtccatgatgagatagagcatcagggtagcaggagcgtgtggcagaggaggctgctcacttcatggtggccaggaagcagagagggaacgagagaaggttggggagaggaaagcatccaagaacaaagtaccttctaggacacattcccagtggcctgcttcctctcccagaccccacctcctaatgttcccagcataaaaaaatagcagcgccagctggggtcagacgcgcagtaggagacatttgaggtccatactgttaatactccatccctaccttctgaaggccatatacttagcttgtagtataaaacacactcagtccatgtccaagaatccagatcctcattagtctcccgatactcaaggcaaactctcagctgtagcccctgttacagtcaacagattatatgcttctccttttcagtggcataaacattctcatccaaaggtaaggaagggccagaccaaagcaaggtgagatggagcattaaagtcccatagtttgacatccatcctccaaggcttgggtggcatgagctgagttccaaagagcttgggcagcccagctcctgtcaccctgctggatggatccatatgccttctctcttggctcactgcctgtaactgtccacagcccacattctggggactcactgggacatccccctcacagcttcatggatcacactgtcaggccactcacagtgattccaaccctggtgtacactgccctgcttatgccaccccataatccttgggttctgcgtgcctcgaaaaactagcatcgtgctgccaggctgagcagtagctgggtctttttggaccgtggcttcagagacctcagagtagctaagcactatgaccaggtcctaaccagccctcagtgggcagggggctctgaggtacctgttctctagggtgttttggacgagtttatactttgcatctgggcctacagtagttaggggtctctccaatatccagcatatttggtatctttcctattttttcagtacaaatttttctgtttgttgttattggtggtagacatttgttttgttttgttttgtttgagacaggctttcttcgtagcccacgctcacctggaactattgtgtatttcatgatagccatagcctcccagtgctcctcttgccttggtctcctgagctaccgtgccttttttttatgctgtgttgggtctggatcccagggccttatgcatgctagaaaaccactttaccagctgagtcacatcgctaagcagttctgacaggtgttttgagtaaaagtaggagtttacctgtgtgcgtttagttttaactgggtgaactggaaatcattagtctagtgggcctatccatttttcttaaggtcccaaggagttctaaagttctcttcactactctgtcctgtggtttctagaactcttttcaacgcctctaaagaacatacggaccccactccagccagcctagaaggcctgaggctgtagcctgtgtggtttgtaaatggaacactgatgagacattttgcagactgctctttcgtggagttcaattttctccatgggtttctgtttacctcacttttagtcattgtgcctttgaactagtgatcttagcttctaagagctgcctaataagactttttgaggcatcatagatctgttaggtgtgtgctgtcctgtgtatggctcatagttgaggagcccgtgtggttaatgtaacttgatgattgattgacttaacctctacaattttaaagtttataacctatcagtatcaattaggttcatggtgttggacagtcgttgccactgatgactgtgctgtgacttagaaaacccttccatccagcttaataagagtggttggtgttctagtggttgtgtaaagaacaaagctcttctctagaagcctggtcggctataagtatcaagtgaattcagctgatagagattaatagcaaaagagggagcagggagccccagactgtctctccgggtggaacccttacttactttgattttgaaatgtgtggttagctaggtatggagtcactgatgccagcacgtcagcttcagctcaaggaccgtaagctgtgtggccttttctttgctgtatccaatatagacaatacattttcataccagagatcgcagacttgacttgttattttcctgtgtaatcaagtataaatttccttcacgcataaaagacctttcccccattgtagtggatgtggtactcctaagacgtgacctatggtcaggagaggcaactcagcagttaaagagcatagctgctcttccagtctccaggctggtgttcagattctagcacctgcaagatagttcatctgtaactccagtctcggggacccagtgtcttcttttggtctctgtgggcaccaggcacgcacatagtacagataagcacacaggcaaaacgctcgtacacataaaaataaatgattttttacagatgtgaacagagggtggagggcttctggattacactgctgctgctgcactgatctgcgtactgtcctgggtatattagaacagtctcagagcagttcgtccttcttcaaacatgctacggaacatcacagatagacagtaccgtgctctagtgaaccctgtgtctgctctgcccagagggtgaagtaaaatgtgggtggttgagcaaacgcactggagtgcacagcatatctagctacccattgctctccattgttaagtttccggtcttttcctcatttttagttttttctttccttaaactcagggagggcttatgtatgccaggcagatactgtaccattgagctgcatctctagaagttggtctcagttttgaaaagaagttggaccgggcgtggtggcgcacgcctttaatcccagcactcgggaggcagaggcaggcggatttctgagttccaggacagcctgggctacacagagaaaccctgtctcgaaaaccagaaagaaagaaagggaaaaaaaaagaagagaaaagaagttgcactgctgatactaaagccagtgccttagggttggaaagatggctcagataatttctgaattaaaatttggtattatcggggctggtgagatggctcagtgggtaagagcacccgactgctcttccaaaggtccggagttcaaatcccagcaaccacatggtggctcacaaccatccgtaacaagatctgatgccctcttctggtgtgtctgaagacagctacagtgtacttacatataataataaataaataaatctttaaaaaaaaaaaaaaaaaaaaaaaaaaaaaaaaaaaaatttggtattatcatcttacttgaaatacattttttttaagatttatttattaatatatacactgtagctgtcttcagacactctagaagagggagtcagatcttgttgcagatggctgtgagccaccatgtcgttgctgggatttgaactctggaccttcggaagagcagtcaggtgctcttacccactgagccatctcaccagcccccttgaaatacattttaaactgatgataaacttacttctttgtggggaaggaaaagctatcccagttgtgaaggatagctcactgtggaaatagcaggctgtactaagtcttgctgtgcactgtcacatagctgggtggcagctttgccttagctcctaaggagggttttcttccataatgtgtttattaccttccaattcccaggtttgctgtagcagctccagccacagccctcctcgctgtgcacgatgttcattcatcctttttactacttttttttttttttaaataaactgcatagaatgtttttctgggttttgaggataggtcagaacagtgagtcaagtgtttggatcagtcggtgtttgccaggctggctgcactgggcaaggctggagagatgctgctgccaccctcaaaaccagggtagtcagtctgaaccaaccagagtggtggggtgactgggacagaagtcttcctccggtggaaaagcagaatggaatgaggagtgagaggtgcaggtaagttacttcagagagaagtccaacatggagtaaaaccccaggtcattatggagaggagacggagactcaggagtccaggtcacctcagccacatagtaagtttgaggccatctcaaaaaagcttgtccagaaagacttcttgttagaacagcccttggaagggacagtgggaaaggaaaaggcttaatacaggaagagtggaggcttcccttacaactgcataaagaggcatagaagtgcctgagacctttgccttgagttgaagctttgagcctattcaagaatagtagtttgaggcaagcagataaaagttgagtgacaggtgtcagaaaggcgtggatgtgagggtgaggagctggttgtgatttagtctgagaatgctcacccctgcagacaagggagaaaggtatggatgagccctgcctgctgatagatgcatgttcttcaggcagcttggaggtgaagctaagtcagttaggaaacaacccagacttcacttttcccaaagggaatatgaaacttgcctctttagaaagtccagctgggccgggcgtggtggcgcacacctttaatcccagcactccggaggcagaggcaggtggatttctgagttcaaggccagcctggtctacagagtgagtgccaggacagccagggctacacagagaaaccctgtctcgaaaacaaacaaacaaaaaaaaaaaagaaagaaagaaagaaagtccagctggttaataaactactttggagtcgtacatggtaaaatcacctcttggagggctggaagagtctaatttttttctattaacatacatcctcttgaagtggctaatcagatattcctggggagtagaaatgtgaatggagctagatttatataaactgagtagcttcttcctgtctgcaaggaagatgctcaactgtcaccattagcatgtcggcagtgttacggcctgagtgagtcagtgccagtctcagtccacccactgtccgcacctggagcatctcctcttttccattttcagtagcaggggttttgttttgttttgttttttcttttaccattctaacttcagaaatatcttgtgtgagtggacttccttctgggttaactcggttttctgtaggatgccatttttgggcactggctcatcttatcttaaaaattattgactgttgcaggcctgtgaaaactggtgtgtaaatacacgaatgcacggccttgttgcattccaggaaaactccatgtataagacaggcaccaggctgtatcttcctgtcacctttctagaattttcttctgttttagcaaaaaaaaaaaatgttcagaggcaaaatggccatcttgttgcataagcagttcctgtgactaggcaactgtggcttgcagacactgttctcttgactgactcatgcttattttgcctctctatatgcaagctaaatcttctctatctgttctttaaatattgtatgtgccttttatgtatcagataatctaaaataagggatggggttcagccatacattgtagtcccagcacttggatagatgaggcacaaggactctgagttaagaggctgctgggggattttaggtgacacaggaaggacggctaatgatcctgcagaacctgacgggctcacacagtctccctatctcaccatcagctgaggtatggagcagctgttcagatgctcaaaaggggctgactgaaggcagtgagagtaaatggcctctgatcctccggtgaacacatgctcatttgttctgaggttttggatggcagaatgactattccgggccctttgcctttctgcttttcaagtctttagtgagtagactttggttactttacatccgttatggttgcagcctggtgtctcccatagtggggtagcaccctctgccagctggctgtccctcacatgttctgtctcctagaggggagaggggaaggtctccagagggcacactcaagtgaacgaatggtgtctgggaggagagcaggcctcccaactgtcagattacaagttccttaggtcaggccttagggggatagtcacaaatagatgcatagagtgtgctccgcctctgcttgccagcacactctgcttagctacctcctagtgccttggtggaaggggaggtggaggtggaggctgggctcatggactgagtggcgtggcctcttgtctaagacccaaccacagtgactttacttcctgctgcatggtggtgacaaatgtgtgtgtgtgtggcggttttttgaacctttctacaggaggagcaagtcatgctgaaagtggccaccctacagaagccttccacccagaatcttccaccatgttggccttgtttgggggagggggggactcatttccattattgagattttttttgtttgttgtgttttattttgctcattattgttttgttttgcttttcacttttattttgtcccgacattttcctttatagtccaccgttagatattgatcagttgaagctcattttaagactcgttggaaccccaggggctgagcttctgaagaaaatctcctcagagtctgtgagttgacgctttgattgaattactaccctcgggggcctctgccacttgccttccgtcactctgcactctgactttggaatgcatgtgtgacgtgtgctgtgtgtgatgtgtgcacacatgtgagcctgcatgagcccgcttgcatgcatgagtgtgttttattctaaactatccttatctgggtgtggacttaagatcatgggaacccctttggggcaattgtatgctaataaaactgagagttgagattccagttcattccaagagattaatgggaaatttgtgtgtgtgtcctcctgagccctgacttctctctgaagttgcccacgtgcttaagagccctctcactttctgctgagcatcctaatctctgacactgtgtgctagtctgaccttccctctcagtggtgggtgatgtgtgccttgtccctagaggggctcctcctgggcggggtctttccatgggctcctctcccaccctcagaccacacagaacagcttcctcccctcatgacagtctgcagatggctctgaactcctccaggaaccttggtcaatacagtgaatagcatgaggggccacttggaacttgtgctttctccatggaggccataagaaggtgactctgttttactttagccatggttggttggttttttgttgttgttgttggttttttgttttttgttgttgttgtttttgttttttggtttttttttgttttttttgttttttttttttgtttttttgagacagggtttctctatgtagccctggttgtcctggaactcactttgtagaccaggctggcctcgaactcagaaatccccctgcctctgcctcccaagtgctgggattgaaggcttgtgccaccattgcccagccttgttgttgttgtttttgttgttgttggaggattttttttttgtttttgtttttgttttttaagctgttgtcactttctggggccagaaactttgaggaagctaggaaagcccctccttcctctcatttccgcttagggaaataaatggaggagctaagccccagctgtgcacgcctaggttcccctcctaccacttccaatagaagagccttctgctcagctaagaaggaagttaagtagctgtaggcaaccagcaacacacgagtcacctcccctgacaaaatcaggaccccgaatctgcagtgtagctatacataaaggaccctggagcttttgaatgggcctattttattttatttcctttactcctttaagataagaaagcaagccaggcatggtggcgtacgcctttaatcccagcacttgggaggcagaggcaggcggatttctgagttcgaggacagcctgggctatacaaagaaagcctgtctcgtcaaaaaggaaaaagagagagagagagagagagagagagagagagagagagagagcaagcaagcaagctggatacatggcctaataatgagatgtcttttatcttaacatgaagtgggcacatcagatatttgaacagttctagtgcagcaatggacagaatcaggtaccaagacaaacatcctctcagtgataactcagttgtgtacagtagtgagaaaagagcttccctgcataatcgtcagaaatgggtaatggcagtctaaatgttggaaaagtatgggcaaggttaagttagtgttgatgacaccacccagtacttttgaaatctccaaagcaccagctgtgatggtgcaggtctctaaccccagcacttaaaaggcagaggcagaaagcctgggcttaatttgtaagacacctttttaaataaacaaacaactattgtagatgtagctcagtgacgtcaacatggccatgggtctaaccccagaccccctgggggtggggagcatacagtacctacatccgggtgagataaagctgactgagtaaaagtagtcagaaaaatttactaaagaattgtagtcaacagaacactgaataactcattccttctggaaagaacagaacagggaagccagtgtagacaaggacacggggtctgaatacagagaatgcaggacagggagaacaggacagaaagctggcagaggcccgaggacagctcactgcttagtgtgctggcacctgggttaaccagccaaggaaacaaaggcatggctcctgccctcttcttcagagatacatcttcttgactcccagatgacaaatggctgatcctgtacgtgctgcccgcaaggagagcatgacagttgagcgtgtgagctcacctgaatcctgcgtgtctagttcaccctgtttgtcccctctctcagtatgcagagcattgttgagtgtggagttttcagagggaagtccaaagtctgtacagagtgagtcagcccatctctgcactcaggtagaagaagggcaaatgcatagcagtagtcaccttccagcagggttcgtgctgatgtgcaaggaaaggacgccattggcttgtctattaaagatgagattccagctgaaggatgacttgatacttaggtgaggagaaaggaagctcactaaatcatgttaaggtttagtttaaaatactatcatcaccttgagagggagcgtgggataaactggcagtgggtttccatttcagaagctgtggcctgtacctggtgaattggtggcctggctgagggaagttagtagtcctgtcgtttgagtaaggacactgagatggcttagcagcagagacctgagttctctcctagggcccacacagtgggcagagaattctacaacttggagatcaatcctctgatctccattcattagtggtacgtgtgacctgcccctccctaccacacaaatgaacgtgaaaatggaataacggaaacttcatgtaactttgccccgtcagataaacacagtgttttaaactttttgttcattgaagttagtgtcttccctcactacaggtgtgattgtggcatgttctgaagctgtcaaccagactgtagcttcggttttgccttcgccatcactcctaaagtacccttcttcaccaacctatatatttgtggcatgcattttaagtatatcactgccaacgtctttgagacttggtgtagctcagcacccacactccaagtttctcagcctgtaggggtgtgtctcgtagagcctgtgccatcaacactgaacctcctccccagatagatctgttagatgtgttaggtttccagagtttcatcaccattatctccatcttctacttttgccactgcagcaaacaggttttagccattgggacttgaaaatggttctctggctggcatcctggatcaaggcacagtctgcttccagagaacctaaactatttaacattaaagttgatacttaagcaaaagtttgttttcatgtgtgtgtggccactcactggctacatgcctaatagctcctgccattctccacagaatgttacaacacgcagccttaggcgctggcctggtgtggttgggagttgctttgcttgggatggcttttgacttaaacaactggagttgatttttacaccgtgtcttaacaatgcccttgactaggcatcttaagatattaaccagcttcagcagataatgcgtctgacggggacaccccctgcttatctcattaacaggatgccaagccatgaggtgagaacaaacagcatgcacagggaagtctacctcggaggccaccttctcgtggtagtgtctgtgtatagccagcagtttctaatgtcaccgaatgcttgcatgtgccccaagaaccgttaaagcagtactggctgtgtgctagcggagtgttggcatttaggatgcagtctcctgagcctgcgaggcagcgatgcagtgtagggcagtgttccctagtgtttggctttctgatcttgtgcttgaggtaacaagtgtcgttgcagttgtatgtagttagggtgtgctacagccgtgtcatgggtgcatggaacagagttcattagtgtgctttgctctccacccattttacaaccaagagaagactgcatgcaagcacgcactataaaattccttgtgctaataggtgtgctgggttttttttaaacaagacaagtaattcatagtaagtatgaacacctggggaatcgagctcttgaccaggtcataactaacctaactccagtttatgatcagacgtgacagacaaacccttgtagagcattgtgtgcatttcagctcttgtctgtattgaaaaatggcttttcttacctctattcctttttccattctacgtggttaagtaggaagggattgcagccatgtctgtgtctggttcctacaacttcaaattgcagtgagttcgtctgcttcagtaccaactcttcttcatgacttacctgattaggaaaatggtcctggaaaagactgcttggtctcaattctggttttaagcttctgaccctttgaccagaaagctagtaaagagtagctgattgattatcactctcatccacaggaaacagaactctggggagaccgcaagaataaagtcagtggtcacaaatagagggggtcagtggctagaagaagagtaagcctgaattgagcatcccagacagtggtccatacgggcctcagcctagctcattccctgagatcactaacactactgaacatagtcattctgaaagtctgtgtttttacaggcaagaaactacattcagtctctggcccagatgccgaagatgaacttcgcaaatgtatttattggtgccaatcccctgggtgagttgatcgtactttgattttgtggtataaattagaaatttgaagttttggttttgactaaaaagagtaccttttttttttaagatttatttattattatacataagttcactgtagctgacttcaaacacaccaaaagagggcgtcagatctcactacagatggttgtgagccaccatgtggttgctgggatttgaactccggacctttggaagagcagcccttacccgctgagccatctcaccagcccaaaagagtactatctttaagtaacttttgggtcttaataaatatagctgttactgtctgtctgtattgtcacataaggaaatatcatacagtgataggcttgtatgcttatatgcttgcagaatatggtggagaggctaagggaagtgaggaatggggaacagggaattatggggacttgggggagaaggaaggaggtgtgaaccttacagctgctagtggtaggtggagcccagctgaggacagaaaacaggcacagacacatggttaatgcccagcagtgggtcctgatgtcctctacactcacagactcgtccccctttcctcccctctctgcccttcagggtgtaagcagctgcctttgcagcattgatttggtggctggctccttgccttcctgcctccattacctccaggatcacctggcagaggaagtgacagtggtagctcagattgtgtcagcacaggacagtgtgctaaggttctaattcctctctctggtgtagtaggcagtagtagatacagagccatcagaccaccaccgtctcctgccatgagaagggaacaagcaggtggtggctgctgtcccctgcctctcccattcctgcagatgttactgtaaagtcactgcacttaagtcttcgtgggtcatgtccctttctgagggttctagaagccatgtgccagagttacttatcctttgaagggctgatgtctgtggcagtgatttcaaaagctgggagcggaagctttcacgaactctgtccatagccacaatactgaggagtggctgtggctgtggcaatgattcctgtagcaggagaggtgtgtgcattcttggaggagggggtccagcctatgctacaccattcaggcctgaggtgtacatggtactgacaggttgggttaggtttgtggcttccctgtcaggctcactgggctctctgtaacttaccttgagctattgcatcagataagggtctggggtctctgtgatctctaatctttccatgttgattccacccctgaatcctcaccagcagatgtgcggtacacagtgtctgagtcttctctgatggagctggtagagatccccatttagacactgagaagagctgccttacaggagtgaatccaggctgctggagactaaaaggaggctctggcccatctggtgctcttcacagcctgctcacatccaaacgtgcctggcagcccattgccaagtgctggtcagaaggaacatgctgttaccgattgctagcctagccgtccccaacaggctgcacaccagcgatataaatattaacagccctgttgtcggtcacaggcttcgcatcactgctagcctcaagacacagctgagaacttcacattgccctttgatcttagagtgtctatttatgtctgcctccatacttgaaaagtcttgttcttacttctgaccttgttcctcatctaagtgctttgatcctgaggacgtggacctctttttaccccagggtagcaggcacccactcattcatgttcagtataaagtcagcggatttgtagaacgtgaggatttgtatccaggaagtccaccagtgcttcagtgttgaagacagagtcatgtggcctgttgtgccaccatgctcactgtctgcagtgctaggtttagatccaaggccttatgcacactttgcaaactttactggctatatcccagccctcctccagcactctaaaactggagagccagcgtatgcatgtttgctgtttcctctgtaccctcaaagcctgcgtttctgtggataactggtgttcaggctacagggtacataatcactcagaggagcagaggtatagcatctcttcaggaagttctcctcgacacccatggagccttccacgctgaagaaatcatctggagctgtagagatggccaacggggcccttggtctgagtccctgcaccaacaggatgcctcacaactgtctttaattacacttacagaggatccagcgccctctgctggcttttagattttcagccagcaggcatgcactgtagtggagacatacttaaaacttaaatctagctgggcgtggtggcgcacgcctttagtcccagcactcaggaggcagaggcaggcggatttctgagttcgaggccagcctggtctacagagtgagttccaagacagccagggctacacagggaaacactgtctcgaaaaacaaacagaaaaaaaaaccttaaatctaaaaacttgtggagtttatttctgttttacttttgggagagaaaaaacctggaagccattattgaaagtaactgataatcaccaggagacaaaccatctccccatattattcacttggggagaaacggggaagagctcaagatgccaaccaccttagctcagcataaatggagctcctggctgtggtgccccaggtcacagcatcacctgtggcatggtctcacccagtatgcctaatttcactcttaccaacaattgaggtggtaaatgtaaagactagagagaaatgggagagagtgcacacagcataaggagaggggaaggaagcctccatggtggtgtgccacacctgcagtgtccacaaatgaaaaccaaggtggatggagaaggagagctggggggtaggagggggtgtttgtttattattctgggtttcaaacccagaacctcagtaatgtttttaaaatgtgccataggcattcaatgcgtacatggagggaaggggttacatgcagaagcagacaccttaaaggatgctttgtgtggatgtgaaaggtgcttggaacttgcacagtggtggcactgatgtcatgtgttttcctcttttctggtgtgacagctgtcgacctactggagaagatgctcgttttggactcagataagaggatcacagcagcccaagctcttgcgcatgcctactttgctcagtaccacgaccctgatgatgagcctgttgctgacccttatgaccagtcctttgaaagcagggaccttctcatagatgagtggaagagtgagtccagggcaggctagcgtgggctttgagcttgctttcattttattcatgttttgtttgttttctgagccagggtttttctctgtgtagccactcagaggaactcactttgtagatcaggctagccttaaactcagagatccatctgtctgtgccttccgagtgctgagattaaaagcatgtgctaccaagtcccgtttatgtaatattttttaacagtgctggagattggacctaggactcacacatatttccaacatacctatgatcccagcacttgggaatcagattaaaagtttaaggttaaaaaaaaaaacagactgaagccgggcattggtgacgcacgcctttaattccagcactggggaggcagaggcaggtagatttatgagttcgagaccagcctggtctacagagtgagttccaggatagccagggctacatagagaaaccctgtctcaaaacaaacaaacaaacaaaaaaaaaccaggctgtaaagttattcacagctgtgtaggtagtttgaggctatcctgggctacatgagaacctgtctgaataaaaccctcgtggatggttgccagcagctttctgagctcactgccagggtctctgatgcgtatgttccctgatgggctgcagacacagagtgcagagccttcaaactgacagagctttgaggtgcttggcggttttacgtttacagcatcttttcaggataaagagggtctggcgtatttcagcacagaactaggacaggagttttttatagcttacttttggcctagttagggactgccaggaatggagccacatctccacccccagtcatactccttcataaagcacaaggtcagttctgcagttaggactgcctcgttgtgtgcagagtgcagtgctgctccagggagcactcagaaggagcaccgtgcatgatggagtggggagacagttctgtctgtgtccttgaactggagctgctctgctgcagggcagggcaggagcaaggcaggtgatgtagttcatcagaccactgccaggagggaagggctgagtgtctgcctgccggagttgtacgcctcacccttccttccttctttccttgccctaggcctgacctatgatgaagtcatcagctttgtgccaccaccccttgaccaagaagaaatggagtcctgagcacctggtttctgttctgtctatctcacttcactgtgaggggaagaccttctcatgggaactctccaaataccattcaagtgcctcttgttgaaagattccttcatggtggaagggggtgcatgtatgtgttagtgtttgtgtgtgtgtgtgtgtctgtctgttcgtctgtccacctatctttgtggaagtcactgtgatggtagtgactttatgagttgtgaatggtccttggcagtctgcctgctttctcagagtctgggcaggccgatgggaactgtcatctccttagggatgtgtgtgttcagtgcaaagtaagaaatatgaaaatatccctgttcttagttaccttgccactttggcttctcctgtggccctgcctttaccatatcagtgacagagagaggctgcttcaggtctgaggctatccctcagccatgcataaagtccaagagaaccaactggctcctggtctctagcctgtgaccggcttgcttaatgtcctcagaacctgacaggtatgttcaaaactgtcagtctgtttgtgccttaaaagggtgagaagggcgcgtagatagttacagagtctcagctgctgacgttctgagccaggcaagtgcacggggctgttggatggccagtggggagctggaaaaaacaaggcagcctttaggaaggccatggtgcatgtgtgtgcatgcgtgtatgtgcagccgccctccctcacttcaggagcaagctgtttgctgtgcttacccttcacctcagtgcagaggtctccagtgccgagcacaggcacctgccatcagtagttcctgtgtcatcttcacatctagcagagcacggatgtgtttgcatgctgtgctcttggagcttgtcctgtcttctggaagccctggacaaggcgtgtgaaggcttcccagaagttcctgtccacattgcctccgcccaccgacgccatgggcacactgctccctcctcctcctccagctactttgtgttgaacacaattgattctccaggtgctcatggtgcaggaaaacaggacagacagagagcacctgaacccttgccatctgatgtcaccaattcaggaaaacgagtcctctcctaggactatccccggttctggaaatcatgttctcctcactcatggtgacaagctaagaaagctgaacaaagggagagacgagagcgcctgaagccaggagctcctttactatctttctcaaaagggttgttagacacaaaccaagtcatcaaggccccgctcctctcctcggaagggtcccccaccccccggcagcttgacactgaatccagtgtcaatttggggagaaagcagttttgtcttggaattttgtatgttgtaggaatccttagagagtgtggttccttctgatggggagaaagggcaaattattttaatattttgtattttcacctttataaacatgaatcctcaggggtgaagaactgtttgcataattttctgaattttgagcactttgtgctatataaggacccatatttaagctttgtgtgcagtaagaaagtgtaaagccaattccagtgttggacgtgacaggtcttgtgtttaggtcaaggtgtctcctctcagtgcagggacatgcctgctctgtggggcaggcgaggaccctgaatcatttggagcccagaaggaggcagactggccaggtctcaccacctcagtgtgcagttcaactccatgccatcccatcaagatgggttagtagcagtgtctgtttttgaatgccaagtgtgatttccaacaattctgctctggttatttcattgaagacatctttgcacatgtgaccatgctgtgttaggggctgtgttccagggactggactcgaagctagaactggcagaagagttctggcatccacagcgcaatgctgccaccacccagtttcttcatcagaagacaagggaacgagaaaactgctgttcgtttgtatttgtgaacttggctgtaatctggtatgccataggatgtcagataataccactggttaaaataaagcctagttttcaaattcagttggtttctcaggtttattctgttcctcttcttggttgtgggacttttgttgtcagctgtgacctagcacatgtctgacagaccctttctaaagctgcttaggtggacctgaggggaaggcaggtctttgcagtgagtgagttactgtgcagagagaaccgcatggggagagtggcctagtgtgcgtccacacgcactgttcttgtacagtctgaaatgaatgtttttcacatcttcaagtaattggtttgatcagagaatatccacagagtttgttacgtatcttctgtggctactttcctgataccatgacaattgaaatccgaggggcgaagactgcagggctatccaggtagctgcttaatgtttagcaaaagttgctttctgactaaatactctaaaactttatacaccatgtatttttatctttaagttgccaaagagaaggctaggttgtgtgcctgtgagtgcaggatgggaggacatctgtttgactctgctgtcttcatgaccctaaaattgttcttcagaactagtgagctgtatcatgcatgagaaggctgcagggcctcacagcaggttctcaggctttgatgacac

SEQ ID NO: 6 - protein, 330 aa, Homo sapiens
MSDSEKLNLDSIIGRLLEVQGSRPGKNVQLTENEIRGLCLKSREIFLSQPILLELEAPLKICGDIHGQYYDLLRLFEYGGFPPESNYLFLGDYVDRGKQSLETICLLLAYKIKYPENFFLLRGNHECASINRIYGFYDECKRRYNIKLWKTFTDCFNCLPIAAIVDEKIFCCHGGLSPDLQSMEQIRRIMRPTDVPDQGLLCDLLWSDPDKDVQGWGENDRGVSFTFGAEVVAKFLHKHDLDLICRAHQVVEDGYEFFAKRQLVTLFSAPNYCGEFDNAGAMMSVDETLMCSFQILKPADKNKGKYGQFSGLNPGGRPITPPRNSAKAKK

SEQ ID NO: 7 - DNA, 3676 bp, Homo sapiens
aggccggaaggaggctgccggagggcgggaggcaggagcgggccaggagctgctgggctggagcggcggcgccgccatgtccgacagcgagaagctcaacctggactcgatcatcgggcgcctgctggaaggttggtcggggcgggggaggcccgcgtccgccgcgccctggccccctgcccggcccggaagtggcgcgcgggcgggcccccggggccagcttagactgggcggggacgggcagcgaggttccgtggccgcagctgcccccccgcggacgcgcctgagaggtccggggccagctccgacgcgcggagcagctccccccctctcccgggctgggggcgcacgggttgcctgggctcttggggcgagggagaaacccgggaagcccggtccaggaaggggtcctgcgctgtgaggtttttggtggcagccccctgcgcacgacagtcgcctcccgaggcgctgagggtagctgggggcttcccctgggaggcttgaggccgcggtcccgggaggctcagcagcaacagctgcctgtgggcaaggaggggccgaggaggcctaggcgcccccggaggtatccatggggccacccgtgtcgcccaccctcctcctgtcagggtccagggtcctgacaccccattgcgccccagtgcagggctcgcggcctggcaagaatgtacagctgacagagaacgagatccgcggtctgtgcctgaaatcccgggagatttttctgagccagcccattcttctggagctggaggcacccctcaagatctgcggtgagcccccggcccaggtccccttgccttcctaaaagtggcactggaccccacagagttcctggagccctcctcccttgagcgcagtcctggccctgggttcaggtctagaccttgcatctagcttacccggttccctgggtcacaggtgacatacacggccagtactacgaccttctgcgactatttgagtatggcggtttccctcccgagagcaactacctctttctgggggactatgtggacaggggcaagcagtccttggagaccatctgcctgctgctggcctataagatcaagtaccccgagaacttcttcctgctccgtgggaaccacgagtgtgccagcatcaaccgcatctatggtttctacgatgagtgtgagtggtctgagcgtctggggctgcccgggtcctgaaccgcactctttgccattcagtgggaccttcacattggcctgatatctgacagacacttgatagactgtgcgctccacgaaggcggggcccaggtccgttctcgtgcctgccctgcccctcctggcatggggtctggggaatggttgctgactgaagggctgcccggattctagtgctttcccccagggccctgcctggtgacactccccctacatttgctactaagcacttaggatgtttgcagatgggctctgctgtccagaagctgagataaaccagacctggacttggcccggctcatgggtgggggcatgcgaggagtgccaaccctgaggctggtccagattggaacttagtttccagtggactcgggtgctttagaattacttctggcaggaggccgaggcgggaagatcccttggacccagaagctccaagttacagtgagctatgatcttgccactgcactccagcctgggtgacagagcaagaccctgtttctttacttttttgagacggagtttcactcttgttgcccaggctagagtgcaatggtgtgatctcggctcaccacaacctccgtctcctgggttcaagcgattctcctgcctcagcctcccaagtagctgggattacaggcatgcgccaccacgcctggctgatttttttgtattttagtagagacgaggtttctccatgttggtcaggctggtctcgaacttctgacctcaggtgatccgcccgcctcggcctcccaaagtgctgggattacaggtcttagccactgcgcctggccagatcctgtcttctttaattaaaataaaaagtaactagaattgctcctggaaggctgacaggtggcaggggtggggggtggtttgcatcggtgcctgactctctcctcccagccacggctgaatagcccagggaccctggctgagggtggggtaggcaggcacctcacagccacctccccctgggcaggcaagagacgctacaacatcaaactgtggaaaaccttcactgactgcttcaactgcctgcccatcgcggccatagtggacgaaaagatcttctgctgccacggaggtgagggaggggatggctggggaggaggagcactggccgcatccctcagcacccggcagctctccttaggtctcatctgggaagtcactccttccaggaagtgtcccccataccccactgtgctgggctgcactcacttccttgatgtgtgtcctacatggagtgggcttgtctcctttctcccgggagctctgtgctcctgagggcagggatgcctttgtgcctgtttgggaacttgaaaaagctgaggggaaggggatgacttctgcctgtctaggcgcccaggagaccagtggcgggaccaggagggtggtgggaaaggtggggttcattggccaaggcttcctggagggaggatgtccttgtttgaggaagtgaccttcccccggccccccaggcctgtccccggacctgcagtctatggagcagattcggcggatcatgcggcccacagatgtgcctgaccagggcctgctgtgtgacctgctgtggtctgaccctgacaaggacgtgcagggctggggcgagaacgaccgtggcgtctcttttacctttggagccgaggtggtggccaagttcctccacaagcacgacttggacctcatctgccgagcacaccaggtgggctggtggctccggcctgcggcaggggaaggggagggccgtgcctgcgctcgggactgactgctcccatcctcgcccaggtggtagaagacggctacgagttctttgccaagcggcagctggtgacacttttctcagctcccaactactgtggcgagtttgacaatgctggcgccatgatgagtgtggacgagaccctcatgtgctctttccaggtgagtgtggggaagaaaggagccgctaggccctgtggctcagccccatgagcctgaccaacactgtctcttttagatcctcaagcccgccgacaagaacaaggggaagtacgggcagttcagtggcctgaaccctggaggccgacccatcaccccaccccgcaattccgccaaagccaagaaatagcccccgcacaccaccctgtgccccagatgatggattgattgtacagaaatcatgctgccatgctgggggggggtcaccccgacccctcaggcccacctgtcacggggaacatggagccttggtgtatttttcttttctttttttaatgaatcaatagcagcgtccagtcccccagggctgcttcctgcctgcacctgcggtgactgtgagcaggatcctggggccgaggctgcagctcagggcaacggcaggccaggtcgtgggtctccagccgtgcttggcctcagggctggcagccggatcctggggcaacccatctggtctcttgaataaaggtcaaagctggattctc

SEQ ID NO: 8 - DNA, 51337 bp, Homo sapiens
gagactcggcgccgtggacgtggagagagggcgacgcacctgcgcgaagacggcagcgggagaagccgctccagtgcggttgcgcggcggcccaggcgtcgaggggagcctcggcggggagcgcaccgcgcgcctgcgcggagagctgcgtgacgcggcggcgcgcaagggacgtgcggagtgagtggcgctgcgggtggggccgtcggcggcgctggtgagctttgcggagctgggcggtgccgaggaggaggaggtggcggcctgggtctgacgcggccctgttcgagggggcctctcttgtttatttatttattttccgtgggtgcctccgagtgtgcgcgcgctctcgctacccggcggggagggggtggggggagggcccgggaaaagggggagttggagccggggtcgaaacgccgcgtgacttgtaggtgagagaacgccgagccgtcgccgcagcctccgccgccgagaagcccttgttcccgctgctgggaaggagagtctgtgccgacaagatggcggacggggagctgaacgtggacagcctcatcacccggctgctggagggtgagtgcgcgcctggccgcgggacagagggaggtcgggcaccgccgccgacccctgcgtccccgtctgccgccggaacgcgaggggacccctttcccgccccgagacgagtctctgggagcgcggcgcggcggacgaaccgaggagggggcgaggaggctctgggcgcgggggagcggcctctgggagcgcggtcaggggagatcgggggagagggggccgttcccgcggaccctcgggggccaggcccgccggccgaaggctgtgcggagcgggtctgcgggagacagcctttcggaaaggccgccgggctccgcgcgccgtggtgctcgcctccggtgacccgaggatggggagaggatgcctgccactgggctttgtgcgtccgtggaatcactgttcttctctgttctctttgtgcattgcccttggctgcctccgattgtcgttccgggagtgaattttacgtatatatatataaacctgtccttaacaggactgcttcgtctgctctgcatttttacacaatgctatatagaatggattcttcttaaagagaggataaaattagcgggaattaatctaccgtttgttacttggcctaaaaaaattgtaagacaagatatttgatcaggttgaagatgcacctaagaaatgtgatgtatgtagatgggtttgctgaagggattggcttttgcatttgatttgagcagagcttaatttctgcacaaacttagtgaaaatctatttttaaagcctgtcttgtattcgaaaggtatttgtaggtctacgcagcatttgaactttccgggctcaagtgttcttattgaatagttttatgttggtgggaaggcaaggggggaaaacgaagaggtggactagccgggagttaaatgtattgattcattgcaggatattattcaaagttaaatgcagtcttctgccaaacagtccctctaccctaaagttttcgtcatcaataattgttttgtgagtgtgtagaaaagcctttaatcctttttaaacaatttagaggcctctttgttagccgccgtccagcgattgcatatgtactgtttgctttcaaattaccttaagataattgctagagtgaagcagtatgtacttatatttggaaaatcatcaaagaatattgtaatgggggaggggtaattcctttttctcaaagtatgtgtcggtatctcaaacccaagttctgtgagtacctgttctgtaactaaatactgtgtatttgctccagatgtgaaagccatccagcagtcttaagactgaataggaagcaaaagttttaccaggaactcttatttaccttttcctttactatcaagttattcctgggaggccccctggtgcctgcagtcagtgttcactacctaagactttgtttcagatgtgaacatcttgctgggtctttgagaaatttgctttgaaaataattgtattttgtaactgttttatgagggaaatacagttaatttaaaaatgtgtatgtcttgtttacatttgggaagtagaggcctactcctttttattttttttttatttttattttttgagacggagtctcactctgtcgcccaggctggagtgcaatggcacggtctcagctcactgcaacctccgcctcccgggttcgagcgattcttccgcctcagcctcctgagtagctgggattacaggcaccccgccatcatgcccggctaattttttttgtatttttgtagagacggaggtttcaccatcttggcctggctggtcttgaactcctgacctcgtgatccacccgcttcagcctcccaaagggctgggattacaggcgtgagccatcgcgcccggcatatttttatatattttaaactttgcttgtaaatttaaatattttaaaaatatgtttgtatctcaatttatttatattttatttcattttccatatattttgtaggcataaaacagatttatgttttaaaatgtttgtatccttttaaattagacgcacctttatatgattacaggtacttaaacttcacgagtgggttgtgcaaggttactagaaaagacactgggggaaaaataagtgattccatttgtttttgtgaattcaaattaaatatttggtttgtacaatctgttttccttttttgggggggcggggggggggcgggtaagaattgtagtccagatacgtttctctcttaatttagcaagcgttaacaagttgacctcctccttgccaggcaccagcaatcctaagataagacacagtctcttcactcaaggagctcccagtctaaacaagtgtctttcaggctttttcagcctcctctctgtcctatgttcagcacacattctatcagtacttcttcatcctacagaagcatatttagtttgacagtgcccccaagggattcttaaatattctaggtttttcagctccttcccagatccagacctgttgaaagacttgtccagagcagacttgaattttctagtaaataattatttttaatcttcaaagaaaattttgtccacctggattttcagtgatattagatgctagttagtgatattttgtaaagctgcctagcaagaataactatggttagtgtttttagtgtcaaatgaacgtgtaagtaaaaatttggcaagaatttctctgcattaggggaaaacattcagcaggacctgatctcattttaaaatgctgattgtagccccacaacatattcctgattgttttttcttttttactgtattaaatcgcagatcagctatacacacttctggtgactacattttatgagttgctttttgtttttcttttttaggtgggcgtattattgataagttttttttgttttttgtttttttgagacggagttttgcgcttgtcacccaggctggagtgcggtggggtgatgttggctcactgcaacctctgcccctgcaggtccaagcgattattctgcctcagcttcccaagtagctgggactacagcatgccaccacgccccggctaattttgtatttttagtagtttcaccatgttggcctggctggtctcgaactttccatcctcaagtgatccacccgcctcggcctcccaaagtgctgggattacaggcatgtgccaccgcgcctgaccttgatatgatattttcaaagagtcctttaggtcattttaatacaaacatgccatttaatgtaaaacagaaatattgattttattatttggacggtgcatattctttcggtatgttttcttggattaacttttgcttagaactaaaaaccaaagacttctactttatcccctgtagtgaccctgttggccacattcttgtggcttaccatagtgttttgatcatttaaattgaagctagtttgtatgagtattctggaaaatccggtaaaagcttcttacttagatgagtggttctcaaagaggggtgggaaatgggagtatagcagaatcatctggggaacttcattcaaactataagtgctaagtttattttggaggaagtgaagtggaatgagtagaatctaatgcatttgggaggaggttagtatggaaaactgctgctgggtgaataagagtgccagctctgttcccaaccacagtcttgatccaggtatttaaaaactgggatgtgccagttgatatagtatgtctattatgtgtcatatatgtgataatatgaaactgtttagaatttgctttttcctgattatgaaagggcagcattctttcaagaagtcccctgtaattccacaatccagagataaccgttgttgaaattttgatatatttttgtttaaagatacttaacatactttgtaaaattcacatgctttacatcattagaatattctttacatagctttatgagttaatttttcatgtagtattacatcatgaacattttcccttgtcattgaaatgtcttcaaaaataccgaaaagcgatttgctgtgtgatagaaaagtttttcttaaagtattttttaacatattaccacgaagctgaacttgactattacaatatgagttgtagaatattagcactgtgtaaagttccatagcaaaatgtgattgtgttacatttttggcccacttacagttttatgaaaatcgtgggatgtgtttgtacctgttttgttttttaaattcatttttcatttaactttgacctgtcagatgtctgaatttgtgacactgcttgtgatctcttagtgcgtatctcccttttctttcttgtgaaacaggtatagtactcttaagatttagttaggctgacagactacacttttaacttttcctattcaagacaggctggaatgcagtggcttgatcatggctcactgcagcctcgacttcctggtctcaatcgatccttctgcctcaacctccctagtggatgggaccacaggcacatgacaccttggctggctaattttttttattttggtagagacggggtctccctatgttggttgcccagtctggtcttgaacccttgtgctcaagcaatcctccagccttggcttcccaaagtgttgggattacaggtgcgaggtgctgcgcccagcctgtagtgtccttttgaaggttaaaaaaaatgaatggatttgaaaaatgaaatcgattaatgaaagcatattaaagccctccattttaaaatgacagctttatccagatataattcacataccatacagtttgcctatttagaggatatgatttttagtatactatagagttgtgcagccatcaccacagtgaactgtataatattttcaaacacatataccccccaaaaaaccctgttcccggtagcagacaccccatttctctccattccctcccggcccccactaatctacttttttttgtctcttagatttgcctattctggacgtttcatgcaagtgaaatcatacaatatgttgtcttttgtgactggtttcttttaatgtttttaacattcatctttgttgtagtatgtatcagtactttattccttttatggctgaataatattccattgtatgtatacaccacatttttaattcattcatcagttgatggacattttgttttctttcggctattgtgaatacttgtagtgctgctatcaacatttgtgtagaagtttttttttgtgggcatatactttcaattccgttgggtatatagctgggagtgagatttctaggtcatatggtagccctatgtttaactttttgatgagctgccaggttgtttaaggcctctttactgattaaattgtttaattctgacccagcaacctaaaggttaggtggttggaaagaagtgtcagttgttgccgacaacctccacttctgtagggaaggtgttttgtgtggatgtgcagctagacttgttagaaaaaagagcatgttggggagtgggggagatcacccgaggaatattttatctccttgattcttctggatgagatactagaggagacataaggtcgtggcatattttttaactgattcagagattaggtgtttgttttctctgaattaagttcagcaagtaagggaaactgacagtttgaagaagtagcccagaggtgtttttattttgctatctgaatgcagacctggagcttttctggtaaatatctaaatccaagatgaagattcgttgggatttaatacattatttaaattgagtctagagacaaagccacattaagagtcaggattagtggggaagatagttgcatctagtaggtctttttcttccttattctccacgtttattacttttaaaagagtatacattccaatgccatgcatttttaattttttttaattaattttttttttttttggaggcagggtctggctctgtcacccggttggagtgcagtggcatgaacatggctcacctgcagcctcaaccttctgggctcaagtgatccttctacctcagcctcacaagtagctgggaccacaggcatgcaccactaattaaaaaaaattggtagacacagaatcttactttgttgcccaggccagtctcagagttctgggattacaggcacccagccccattttcaaacatttctaacaggtaaagtaatacaggtatgaaggaatgttaagattcttagaaatactgacttaaattctgttatgtatttctgaaaggttattctcgaatgttggtggcatatagggaggcaaggggaagaaatagtttgtaaatgtcagtaggtttagcagtggtgtggtagatggggattcccagtggagtcttctaggatttgtgattggatacagattgatacccttaggcagcagccactgcttctagcagctttaaatagccatttcaaggcttattcaagtataaataaccagtagccagttttttcctgatttgatagaggaagtcagtccttggtctagatgggtacttgtcatacagtaggaaaaagggcgggggcagttgtcctttattctgcctctgcactattcacatgtggagcacagtgatttgtgagagtatgtataaccttcgtgaacatcactggcctcagaaccctcccttgccaaatttacctgcatctgtgcaaagacaagtggagatattgatgatatgtgcatatcccagagggaatagcgtttgctttttgatgcatgcccacccttgcataattgggaggttttaacatagcatatttcaccataaaggaattttattctaagaatgttaatcagaatatccctaagtaacagaagttcccatctggtcagttgcaaggtgaccaaaacactttcttggtttgagtatagtcatatgttctgtcaaatgatgttttggtcaacagtggacttcatgtaaggtagttagtggtcctgtaagattagaatggaactgagaaattcctgtcacttagtgacatcataggcataatgtcatagtgcaatgcattactcttgtttgtggtagtgctggtgtaaacctattgcactgccagttgtgtaaaagtacaacaggccacgcgtggtggctcatgcctgtagtcccagcactttgggaggccaaggcaggtggaccacttgaggtcaggagttcgagaccagcctggccaacatggtgaaaccccatctctactaaaaatacaaaaattagctgggcttggtggcatgcatatgcctgtaatcccagttactcaggaggctgaggcaggagaatcacttgaacctgggaggtggaggttgcagtgagcaaagattgcaccactgctctccagcctgggcgaaagagtgagactgcatctccaaaaaaaaaaaaaaaaaaaaaaaaaagtataacacaattatatagagtacataatacttgataatggtaataaacagctatgttatatatttaccttttttttttcttttttttgagacagagtttcgctcttgttgcccaggctggagtgcagtgacgcgatctcagttcaccacaacctctgcctctcaggttcaagtgattctcctgcctcagcctcctgagtagctgggattataggcgtgcaccaccacacctggctaattttttgtatttttagtagagacggggtttcaccgtgttagccaggatggtattgatctcccgacctcaggtgatccgcctgcctcggcctcccaaagtgttgggattacaggcttgagtcactacgcagggccctggaatatattcttattttaaaaaactagttaaaaccgactcaggcatgtccttcaggtggcattccaggaaaaaaagcttcgttatcctgggaggtgatggctccatgcatgttactgcccctgaagaccttccagtgaagatgtggaggcggaatatagtgttactgatgatcctgactctgtaggcctaggctaatgtgtgtgtatatggcttagtttttaacaaaagtttaaaaagtgaaaaatatttttaaaaataggaaaaggcttaataggataaggatataaagaaaatattgtacagctgtatgatatgcttttgttttaagctaatcgttactagaaaattcaaaatgtttaaaaatttttttaagtttctaaagtaaaaaatgttaagtaagcaaaggttaatttattcttgaagaaagaaaaatatttttatctttattactgtatatacattgtgccttttctgtgtttagatatgtttagatatacaagcacattgtgttacagtagcctgcagtattcagtacagtaacatactgtgcatcatgctttgtagcctaagagcaatagactataccatctaggtttgtgtaagtccactctgtgatgtttgcacaacaacaaaatcacctcactgtgcatctctcaggaagtttccctttggtaaatgatgcataactggacttgtctgacaccattttaaccttccccctagtcctacctctcattctcaagatttggagaaatcaaaatctaagttttcataataaaaggttaggtatattgggtgttacatagctttcactaatgtcatatctagcagttgtaatcacgtattttaaaactttaaatagtaaattaagaaggaatctttataggtacttgtatgtttcctcaatgtggtttgaattgagcgttttatttaaattactatcaaatacttgagccaaactagcgagacaaaaagaccagaccttttcttttttccgagatggagtctcacactgttgtccaggctggagtgcagtggtgcagtctcggctcactgcagcctccgcctcctgggttctagcgattctcctgcctcagcctcctgagtagctgggattacaggcgcctgccaccatgcccagctaattttctgtatttttagtagtagagatgggatttcactaaatctcgaacacctgaccttgtgatttgcccaccttggcctcccaaattgctgggattactggggtgagccaccgcgcccggccgagaccagacttctgagggggagaaacttgcaaacctgtcagtgagaaagagtgaggtctgattttagatgacagaaaggaatagcttttgtgaatttcggactcccaactactagaattatgtgaatcctgaatgataatcagatgtttactcagttaagtgtttactcaaatattaataagtattgatacattgggatgccatcttatataatgattaacgacacagattctgggtgtatttcagccccagtttcttctgcatatatgatagtaagtgccttcaaacaattcttgacacattaaaggtcagaaaatgttagccgtcattattgcttagtacaaacctgagctaggaaatactaacagagagctctttgtgccactgagtcctggaacttccccatactaattttgagaaggctttcttgtaagtatgtgacagatactgtttcctccactttctgtttattgcatataaccactattcttgtatgtaagtacgtataacagtttctttactattcccattattcagcgtttataatctggtttgacatatcagtgttggtgctgagagggaaaggtatttctaggtaggataaaatggtgaaaagtgatttaacactgttgtatgcttgcttttggccaggaactatagggcagtgtatcttaaactttgagtcttgagaccctcttacactcaaattctccatatatgtaaagaattgaaaacgagcttttatttatgtgaggtatttaacaatattgcattagaagttaaaactgagaatttaaaaatttctgttctcggccgggcatggtagctcacacgtgtaatctcagcactttgggaggccgaggcgggtggaattgcttgaggccaggagttcaagaccagcctgggcaacatggtgaaaccccatctcctaaaaatataaaaaagttagctgggcgtgatgtcacatgcctgtattcccagctacttgggaggctgaggcacaagaattgcttgaatctgggaggcagaggttgcagtgagctgagattgcgccactgcactccagcctgggcgacagagtgagactgtgtctctaaataaataaataaatatctgttctttaagtagcaacatattacattttagcataaatgttttttatgaagataacttttctaaagcaaaaatgtgagaagtgccatcattttatgcaaatctaatgtctttttaacagaagacagctgaattcacatctctgttttgtttcagtcttttgataccttgttttagttgaatatgaagaaagtctgtagtcctacagatacgtagttagaaaaaggaggagtagtttaatagctgttctagataattgtgggtattctctgatgtttataccaaaaatcagcaagtggtggttttttaaaggtgaattttaaaagagatgtggaatctgaaattatatccatgaatttttatgttctcttacattaaaatccattgatctttcttgtactttaaatggatcttacacatgactttataatacgcgttgttcatttggataatattgattcagctgagttatgcagatgtccagatgttgaaacagcttattatctaatacagtatccagaaatgacatttgttaatgttacaagatctcatcagaagagtccttaattatggagaagctgtcagattcatggtgctgattacaaggtttctagaattttagtttgtaacttgaaagcttgaattttatcattggcaacaagtactgtcagttgttttccttgaaatgacagggtcactttgttcatgttcacgtgtatctgttgaatatctaagtctgaataaccatagtttgtcagttaatactttccagtaaaagtgtttagtgaaaaaaagtgtctaggtctgcttggaactaaaacagtcacaggagtgctttcctttgcgacaaccattgtactttgggatggagcaggagtgctttgtgggcatttccattttatcatgtagattattaaagagatgtattcaaagattgaagtttaataaaattgatcatttgtactgctttaccaaagggcatacatgaagctgccttttttgtgtttcactgcatgtgtgttgcaatgaacaacacagtgactgactaccagtggggtttggtgccactgacttggtgtgtactgagacacttaacagttttacccatccttgcttttgtattatcaaagcagatgtcagtgcagtggaaaaaaaaaaagtcctttattcctgaaatgtctagccctcacctatatctgtcacttttcttcttcactcttttctcattatacttgctatttgtttaccaatgtacctgaaatgactttttacttatgctttgtgaattcatgaaagccatgggccttctcagaaaaatatttttacatatacacaatattacagacaatttgtggtactatacagagccactaaaggccattttggtctctgaatccacgttagttattcttttgttttgagacggagtctcactttgtcacccaggctggagtgcagtggcaccatctcagctcactgcaacctctgcctcccgggttgaagcgattctcctgcctcagcctcccgagtaatctgggattacaggtgcgcgccaccacacctggcttattttcgtatttttagtagagacggggtttcagcatgttggtcaggctggtcttgaactgacctcctgatccgcccaccttagcctcccagaatgctgggattacaggcatgagccaccatgcccggcccacattaattattgttaacaggcagtcctaagatgagagggaagtacagcagctgcctaattttttttcacttaaaaccgtgtgtgtgttgtgtgcatagagaaagatggggaggaaaggaagacagagtgggagagggagaagaaaagcaagagagaaagaatgaaaaagcaaagggggctggacgtggtggctcacgcctgtaatcccagcactttgggaggccgaggcaggcggatcacgaggtcaggagatcgagaccatcctagctaacatgctgaaatcccgtctctacttaaaatacaaaaaattagctgggcatagtagcacccacctataatcccagctactcgggaggctgaggcaggagaatggcgtgaacccgggaggcagagcttgcagtgagctgagattgtgccactgcactcgagcctaggcaacagggcgagactccatctcaaaaaaaaaaaaaaagagagactccacattttagccaattgtagaatttttatttttctgcctttttgttgcttaaaagcaggcttttgtgctaaatatggagaaaatgctaggaaaaaattaggatgtctagatttaactgccattaattagtatgtaatcttgggggtgggggttgagtaagagttagaataccagtggtcagtgaagattactaagattttatgaggccaaggcgagtggatcacttgaggtcaggagttcgagcctgggcacatggcgaaaccccgtctctactaaaaatacaaaaaattagctgggcatggtggcacacacctgtaatcccacctacccagtagattgaggtaggagattcacctgaagccaggaggtggaggtttcagtgagccaagatcatgccattatactccagcctgggtgacagagagaccctgtctcaaaaaaaaaaaatttatgagacaccgtgcataggtgttggaattagtgcttttttcttgccatcttttctgttttagagaggtaatgataagtaagaacttgctggggaaagtagcataaggttagtgaaaagtaaaatttagtatacacacaacacacagacacatatatatcacatacccttggatatgtgcgttggaaagacaaacctgcaatggaaaagtttttacactgatataaaatgtgaaatataatggttgagtaaaggacagaattcttttgggagaggatacacattttgcttaattgagattcaaagttaatgttctctgtcatcaaaactgattgcaagtggtcatctcttgatgctgatctgtagtgagattctatgtatttcatcttagaagatgtctttttacacagggacatactgcatattcatcacttggaaagcatttatagaattctcctgatacttgcctttcagcatgtcattacattgcagattaatcacttccagtagagggttaggtggaagctcctcggctgcacagttaaatggattttgatatatggaaattttttttgcgcttgagtcaaggtttgaagaatgctagtttgagtttgaaaaaatgtatctgctgcaaaattgtgtgggaatgaagatctcacttgtcttaacttggcagcacgggttatgtataaagcagtttgctgactgggtatggtggctcgcgcctttaatcccagcactttgggaggcggaggcaggaggatcacttgagttcagcagtttgagacctgcctgggcaacatagtgagaccttgtctttatagaaaattaaattagccaggtgtggtggcatgtgcctatagtcccagctacttgggaggctgaggcaggagaatgtcttgagccagtaggctgtagtgagcctcattacaccactgcactctggcctgggcaataaagcaagaccacatctcaaaaagtaaaaaattaaaaaataaagcaacttgctttaattcagacagtattagttgtcagaatgacttaccacaatggaagtttctcatatatttttttgccttataattttaggctgaatttattagaaaacatttaggggagctttcattctattttgtagaatagaggctacctcgaattaaaaggggaaaaattactagatctgccgcaaaatttaatttctaaaactcttctgtgtttggtaatagtgattgagggtggtggttcttgtttagaaaaaattcagtatcagccctgaatgcaaataaattacaataaagctgtcactgctaagccatcaaaatcctgtgtggttggccaaatgaggatattcagcttttattactaacaaatagtcatgtaactgacagtggttaagttcattagagcaaatgaagttgaccattgatgcctgtggttcagtaatacctgattaagatatttttccacaaaaaaactgcaccagacttcaaacatcctacattttctcagactttgtaaaatagtccaactaagtatttttctactttaccatcatcagttgtgacatatcttagcagattacacttcaggttgtactaaaatagtttttctcagctgctttgagaatattcttgcctctagttgttccacatagattatttatagagggtaacttttcagtcacttcgaatgaggattggctccccaaataacaagtattggtaatacctgtcatcgagagccaaggaacaccactcagaaccatttggcttgttttttaattgtttactaattttgctcccagtgttctcaactcttcaagcaccgtttttacagaaaggctaatggcgtttaaaagtttattgtctcagctggggtggtggctcacgcctgtaatcccagcactttgggaggccaaggcaggcagatcacgaggtcaggagattgagatcatcctggctaacatggtgaaaccccgtctctactaaaaatacaaaaaattagccgggcgtggtggcgggcacctgtggtcccagctacttgggaggctgaggcaggagaatcgcgtgaaccagggaggcagagcttgcagtgagccgagatcacaccactgcagtccagcctgggcaacagagcaagactccatctcaaaaaaaaaaaaaagtttattgtctcaaaaaatacatagttcttcagcttttccaatcagatacaatttaattaacccaccattgataaatgacttaacttggctaacaaatgggccacttggaaacttgagttgcagactcatttttatttttgtgaagaaattctgctgtgatgagatttcattttaattttttaaaaagttcccttaccattgctttactatgaattgggaatgctgtggtgagtgctccatctggtaacatcagtatatgttgtggttttttttttagcacagctacataatgactacaatgccttagcacccaattaaataacataatccacactttattgtgtcttaaaaacacaacattcaaagttcactttttttgctttgacatgatgagtatgcactggtagtaaaagtaaataaaatgttgcagtacagcaataatcgtgacacccatgacgctgccaggttgtaactgcatctgtataatttgtgatgcaccgaaaagcagtgtgaagtgatgagagtaccatatgtagtccatcacaactgctcagctctgccattgtagcatgaatgtgtccagtgataaataagtggctatgcttcattaaactttatggccaccaaaatttgagtttcatataatttttatatgtcacagaggattcttttgttttgcttttttgcaatcatttaaaaaatgtaaaaaattattcttaggcccctagccatgcaaacactggcattgggctagatttgaccccttggccttagtttgctgaatactggtgtagtttttctgcctttcctaatgtaaacatgattgtagaaagataaccttactggaagtcaggaaaccttggtgctcccaccaaagttgtgtgactgattaagtcaggtgaaataaattgggattaggagccatggtgtctagtccttgacaagtcattcctcaatttttttaatgtaatcacctgaatttctagtgtatgcataatcacattttgtgtgcctctcggcagccattccattgttgaagaggcagccaacacagaaaattgttgaggggttttgatctttgacaagggaagcgtatagtgtgagctcaccagtcaccctggcttttttccctggagacatttcctagtttgtggcataggggaaatagatcccaaataaaaagtgacggtctcactgggctaaggaagaaaggtggaagtttggggccactaaagcaattgaagcttaagtccaggagaggagagaaccacataaaatctaacctcaaaatctgcagtcaggtccctctaaagtcattgactgacttcagaggtacacataagcaagagaagaaacccagcagaaaacagtagctgaaaagctaaagatctgagcagacatttcagtatctaatgccagagaggcaaaaatggaagttcaaatcctgccaagttagaggggccttattacaccctgagttttccactgtggccccatcatgaccactgcctaagattgaaggcaaaactaaaacagaccagccctcaggaaaatgagttactgctgtgctgctgctaaaaggaaacagccttctttggagaaaggtattatctaaaacttctacaattttgtatccataattcagtatttcattcaccaaaataggatatgctgttttggacaagctaatgaaactgaattgaccaaaaaccaatagaaattatctagtgctgacagaaaaaaaaaagtgtacagaagaaagcatgagagacttatgtattgctgatagacagtggaaaggtctatcatgtataataggaatcccagaaagagaggagatagacatacaatgggatggaaatattgactaataattttccaaaacagagatatccaagcacatattaaagaagctgtgtgattccaagcagggtaaatagagagaaaaccatattgaacatagctgctagaaatccaagagaaagagaaaatgttaaatgctgtcagaagaaaaaattcattatcttcagggaacaataatatgaatgatggctgatctatcacctgaaactgtggaagacagaagacaatagaatggtgttctctttaaagcactgaaagaaaaaaaatgacaacctagaattctttatttattttgagatggagttttgctcgtgtggccgaggcttggagtgcaatggcgtgatcttggctaactgcaacctctgcctcccgggttcaagcgattctcctgcttcagcctcccgagtagctgggattacaggcatgtgccaccacgcctggctaattttgtatttttagtagaggcagggtttctccatcttggtcaggctggtctagaattctaaatctagaatatccttcaatagtaaagtaaaatgaagacaaactaaagctcagaaaatatattgctgatatgctgacactagagaatattaaagcaagttctttgtgttgcaagaaaataacagataaaagcaaagaactgcagaaagaaatgaacaatggtaaggataaatgtgagtaaatacaaattctgactgtttaaaataatagttgtggcttgtgggatttaaaatatgtggaaacaaaaatacgacaactgtagaacaaaaggctggaggcgggtggtaatcagtcaaaagtgtatttgtaatctctagagcaacctctaaatgaaaaaagaatatataacgaaaagctaatagaggagaaaatggaatagtaaaaaaataactgattactccaaagaaatgcagggaaagagagaggaatgaagaacagatgggacaaacagaacagatgacagaatggtagacttaaactcaacttcgtcgatgattatattaaatgtaaaaggagcaaatattggctgggcaccgtggctcacacctgtaatcccagcactttgggaggccaaggcggacaggtcacttgaggtcaggagttcaagaccagcctggccaacatggtgaaaccctatctctactaaaaatacaaaaattggctgggcgtgggtgcctgtaatcccagctactcaggaggctgaggcaggagaatcgcttgaatctgggaggcaaaggtttcagtgagctgagatcatgccgctgcactccagcctgggtgacagagtgagactccatctcaaaaagaaaaggagcaaacattgaaattaatgcaaagctcagaagacctaactattatagatactgtttacaagggatgtattttaaatctaaggacacaaatgaggaagtaaaaaaaaggggggggggaggggggtgtgggggagacgatatcatgcacacattaatcaaatgaaagctgacttggctatatttatgttggaaaaagctctaaaaccagaaataacactagaggtaaagaagggcattatataatgctaaaaggataaatttaatggaaaaatatctaaaatttttatatatgtaatagtataggtctaaaactaaaaggaaaggttgacaactattataaaaagttaaaatagacaaaaccacaaccatagatcagtgcccctctttcagtaactagtagaacaaacaggaaaaaatcagcgaggataaagatttgaacaacacagccaccctgacctacttggcggacatagaacacttcactcaataatttcaggatatatatgcttttcaaaatatgtatgaaacatttactaaaatataccatatgctataccataaagcacccctcagtacatttgaaagaaatgacatcacagatgtcagccaaaatccatttaatgaaaaacgtgaaagactgccatactagagtatataacattgctgagagaaataaataaaaggagagataccatatttgtggattggaagacttgaaattgtgaagataccaattctgtctagacttacctacttattccctgctcccccccccccaccctttttttttttttttgaggcagagtttcactcttgttgcccaggctgcagtgcagtggcacgatcgtggctcactgccacctctgtctcttgggttcaagcgattctcctgcctcagcctcctgggtagctgggattacaggcacctgccatcacgcctggctaatttttgtatttttagtagaggcggagtttcaccatgttggccaggctggtctcttcctgacctcaggtgatccacccacctcggcctcccaaagtgctgggattacaggcatgagccactgtactcggccttattcccaatttaacacctcacaagtttgggggtggaggggctagacacaagccaattttaaaacatttgaaagttcaaaaggaaaatctaaagaacaaaattgggatgtttatattactagataccgagactttataaaaagccatagtaattaagacattgtggtattgatgcaaaaatagacaaatggggtaatgagctatatagtggctaaaaacagacctgttcctgtatggtcacctgatttttctacaaagctgccattgcggttcagtggggaaaaggatgatctttatgataaatggtgctgaatcaactgggtatctgcttagataaaaaccttgacctttacatcaaaccatacacaaaattaatttgagatgggttatagaatgaagtgggctaaaacaataaaatatctggaaggaaacataaggaaatatgtttagagaaggcaagaatttcttgaataagatgcaaaaggccttaaccaaaaaaagattgactaagattttttaagatttaaattcattaaagaacttctgttcatcaaaagatagcatgaagagatggaaaaagtccagttttaaagaatgaacaaaagacttaaaacgaacttgtccaaaaaaaaaaaaaaagataacctaaagggtctattaggcacatgaaaaagatactcaatggccaggtgcagtggctcacgcctgtaatcccagcactttgggaggccaaggcggggagattatttgaggttaggagttcgagatgagcctgagcaacatggtgaaaccctgtctctacaaaaattagctgggcatggtggtgtgcacctgtaatcccagctgctcaggaggcaggagaatccttgaacccgggaggtggaggttgcagtgagccatgatcttgccactacactccagcctgggtgacggagcgagactccatctcaaaaagaaaaaaaagggtacttgacaaccttggtcctcggggaaatcaaattaaatccaacgaataccaccatatacccaacaaaatgactaaaattaaaaatactgataatttgcagtatcagtaaagatatggaccaacagtgttaggaggattatgaattggtatagccactttgggaaactgatggtatctactacttaatgaaataaactgacccgagcagttctactgctttgttctatacatacctaagataaatatgcccatggatattcatagcatttgtaatagccccaaactggaaataatccaaatagagtagtaaagatattgtcatatgttcatagaatggaatacaggttgaatattttttatccaaaatgcttgggaccccaagtgtttggaatttggatttttttcagatttttgagtatttgtattatacttaccagttaagcatccgtaatctgataatctgaaatctgaaatgctccaatgagcattttctttgagcatcatattagcactaaaaaaggtttcggttttggagcattttggatttcagattagggatactcagcctgtatttcaaatgactgttaaacacaattatatggatgtatctcacagatgcaataatgagcaaaagagcattcttatgtttctttttatctaaaagtctaattcacaaatacatcaaatcaatatatactgatagcaataaggatagtaacctcaggcattactgtcattagttagggtacaagggaatcttcagggttgcaggaagcattctgtaccttgatctggatgatggttactcaggtatatagatagtttaaaattcgttgagttacacccataaagttagtatactatatgtatgtaatatgttaaaatattttctaaacagtagtaagtatggtagatatttgctatcatgggattttatctggtctgaaattactacatgctgaaaatttctaggaaatttttgatccaaaaattttatgtggtgaagtggaaaaagtactggcctttaagttagaagactaagtctgaatccctctctgctgcttctcactcctatcagtcttataaaacctgggcactgtgatcttatgcgagttattgaatctctgtcattgtacagtggaaagaatagcttctcttttcagttcctggggtgtgaaaaggattaaatgaaatgattgatgtgaaggtgctttgcacctaatgaagtatgctgatttataatatttttataatccagcagcgttcagatatccaaacactggaatccctcatggatgctgtgatagcagtctagttgccctgtgtaacaggttgcccggatgatttcttcctttttttcagaagtcttgagagtacataaaggagcaggacttttgaattggaagacagctgggattgccctgggacatctgccttattctgtccaacctttggctaatttatttctaatttctaaaactcttgtcagagttttaaacattgggaaaacctttagcttctctaactgcattcatcaactcttaagatgtgactgattattaaatgcacttttatgtgccactaagttggtggggggggctgccaattataattgtaagaagcttttaattgcaagagtctcacaacttcagagatattagttaagggagaaatgtgcatcctacagttagtgaaaatataaaaagtttctgtgcagtgtgtttttggtaaatttttagtttctgtggatttgcttgacttacaagttgaagcactttatagttctgtaaaaatgttacacaattacttgagaagtgaaaattatacagagggaataaaataaatactaggtaaaatattcaccactgtcatttgaatattttaaattgacatattgaagtgacccacattagtcttttgacttagtagtcaagttacaactaagttttgataatgtactgttcttcttgttcattgctgttctctataaacaactaatacacagtagactctcaaacatttgttgaatgagttaatacagtttgtaagtacttttagctgtgtgtttgttatctgaaagcagtttgagacccttaattcttccaaggcgaatactttttttttgagacagaatttcgctcttgtctcccaggctggagtgctgtggtgcgatctcggctcactgcaacctccacctcctgggttcaaggagttctcctgcctcagcctcccaagtagctgagattacaggcacccgccaccactcccggctaattcttgtatttttagtaaagacagggtttcaccacgttggctaggctggtctcaaactcctgacctcaggtgatccacccacctcggcctcccaaagtgctgggattacaggcatgagccaccgcacctggtcacattttttttttcttaaactaaaatcttccacatttaaaactaaagctgagatgtctttttggtgttagaaaaacccaaatatgcatcagaagtaggtaattgatgtttgagaacaagcactaatgaagctatatcagttcccataataaaagcagttctgctgatcattgttttgaatttcagggatagtcttataaactagcatatttgctaaagcacatttgaatgttgtctgtgtctcctgtttattgcctcctttttacttttatattaatatattaatggaatatatagcttttgcatgtggaggcctttttttttttaaagcctcttattgacctgacaaaggaatcagcaatttttagcaagaaatatgtggaattttattttactttggagatggggtctcactctcacccaggctggagtgcagtggcacgatctcagctcactgtagcctctgccttctggggtcaagtgaacgttccacctcaacctctgccttgagtagctgggaccacaagcatgcaccatcatgcccagctaattttttgtattttgggtagagatggggtttcatcgtgttgcccagggcgatcttgcgctcctgagctcaagtgatccacccaccttggcctcccaatgtgctgggattacaggtgtgagccaccgtgcccggcctttatttttattttgcagagacagggtcttgctctgtcaccaagactgaaatgccctagtgccatcatagctcactgcagcctcaacctcttggcctcaagcaattctctactatgctaggctaattctgttttttaaaaacatcttttgttgacacagggccttgctttgctgcccaggctggtctcgaactcctggcttcaagcagtcctcccgctttgaccccctaaagtgctgggattacaggggcaagctactgctcccagcctattttcttttcttaaatgctctttcacttaagattaaatgagagctcagctgtaaatttaaggttaaagggagtcaggggagatggtgatgagaggaaatgattacaagttgagttcagtggggcaaataaggaactcttctgtctctccatgcaacattctgttccccttcatcccacaagttactggaaaccatattatcttatggctcagtaatgcaaagatgcctcactccatctaaatagcctttagaggttttaatataatgatctattgggaatgtaacgataagttaatttttaatacatagctgatatcttttttgtatttcttttgatcagaaagtagagataggagttgctaaaattttggggactgctggtaacattccaagaaagtgtgtgaagctaaatcacctactgacaattactatttaatctccctttcttcatagcacattgttcttaatgaggctgttttgacatcctactagatgcgagataagcaggccagaccttaaagggtgcgggctctgaaatgtttttttgtgtgtgttttttttgttgttgttttttttctgtttgtttgttttttttttatttgagacggagtctcgctcttgttgcccaggctggagtgcaatggggcgatctcagctcactgcaacctctgcccccgggttcaagcgattctcctaactcagcctcccaattagctgggattacaggcatgcgccaccacacctggctaattttgtatttttggagagatggggtttctccatgttgatcaagctggttttgaactcccgacctcaggtgatccacccacctcaacctcccaaggtgctgggattacaggcatgagccaccgtgcccggccctgaaatcttttaagtctctttggctactaggccccaggttggtcacaggacagcaaaaaacaggactgcatttcatcaacagcagacttgaggagcatcatcaggaaccagtaacaatgaaaatagaagtcagagatcatagaataacattattttaaaccatgggaccaaatagggtaatttgctgcctgtgtgacttttctgatttttaaagtatgggcatgactctttttgaaagattattatgagtaaattttagaaaactgactgttttatttatcgtttgtcagtacgaggatgtcgtccaggaaagattgtgcagatgactgaagcagaagttcgaggcttatgtatcaagtctcgggagatctttctcagccagcctattcttttggaattggaagcaccgctgaaaatttgtggtatgtaaatgggtaaagttggaaacaactcttttgaatcatgtttttcctcccatgtttaagatctagagaaaacattttttgaaatttttgtataaaacagatcagtaggctttactaaataaaagtgtataaaaattgaaacttataaaaatgacaagttacagacaagatgaaaatacttgctatatatcaacagataaaaggagttctatttataatatataaagagttcttataaataagagaaaggcgatacagtataaaaataacataggaagaggaaggtcttagaagaaattcaaatggccagtaaacataaggacgtcagcttctttttttagtaatcagtgatgtgcaatttaaagcaagtctccaactctagtcagattataaaaaaataccaaagattgatgtttagtgttgtcaaggatgtcacgaaaagggcatcatagtacactggttagaaaaaaataggtgtataatttagagggtcaatatggcgatatctgtggtcttctaactttgatgttatttaaatttgttttgaagattatactactttataccttgtattcttttaagttttacagtgacttttttacagtcctttgtacagtgcccagcaaataagagatactcgctaagtaaatgtttgttttttgtttgtttggttggtttttctttgagacagagtttcactcttgttgcccaggctggagtacaatggcacaatcttggctcactgcaacctccacctcccgggctcaagtggttctcctgcctcagactcctgagtagctgggattacaggtgtgagccaccacgtctagctgactttttgtatttttagtagagacagggtttctccatgttggccaggctggttctgaattcctgacctcaggtgatccacctgtcttggcctcccaaaatgctgggattataggcatgagccaccgcagctggccaataaatgtttgttgaatggatcagttaacttagaatatcacaccttcaagtaagaggtagtagttcagaatttgggggaactaatcaatcccctgtataaacttaagcaaatgtagttacttagaggtgatcttaaaattattagaattaaagtattctttgaaggtgttctttaaaatgtaatatagaaataacttgaaggtaatggaagttatttgtactacatatttctaaaggtttactatttgtctccatatttggtgttaaaaagtgacgtaagttaaactaccagataataaaattgttttagttacctattgttgtataataaatcattccaaaactttgtggcttacagcaacattaatttaccatctcacaatttctgtgggtcaggaattcaggaacagcttagtgggtcctctggctctgggtctctcacaaggctccaattaaggcgtgatctcgggctgcagtcatctgaaggttgaactgtgaaaggattcagttccttatagactgatggactaagagcctcagttccaaatgagctgttggtcagaggcctccctcagttctttgctacgtgggatgcttcattagacagctggtttcatcagcgagcatggaagaaggtgagagagtgcaagcaagatgaaagtcacagtcttttgtaacttaatcttggaagtgacagcccatctcactttcgcttattagaagtaagtcaccaaatatagcccatactcaagggtaggggaggattacacaggatgtgaacattgggaatcatttcatagaagctgcttataacagtaaccaatttttctaaaactgactctttctctttttaggagatattcatggacaatatacagatttactgagattatttgaatatggaggtttcccaccagaagccaactatcttttcttaggagattatgtggacagaggaaagcagtctttggaaaccatttgtttgctattggcttataaaatcaaatatccagagaacttctttctcttaagaggaaaccatgagtgtgctagcatcaatcgcatttatggattctatgatgaatgtaagtgaaaataaagacttagaaaagtaattcatggaaacattgcctaataattttctggttcagaagagttgctttgattcagagttttttcaaaataagatctgtcaggcataggccaggaagaggaacctgaaactgggaaacatgttactaattaaattggttccttgagatctccttggtcatatttcagattgactgcagcttacttggctgaagaggctcttttaaaattagtttatctagtaaactatccaggtgaaacagaagtcactgtaggcagtggcccttggttttctagtgtccattaaggttttaactctcggcattcttgtggtatgacagcttttattagtcctgtgtcacaagaattattgagaagttctgtgaaactatttaaaagtcaaagtttaatattattagaatttaaatctatgtctgtaaaccttgaaagataaagatactgaatactctggggtatccttattttactctctttttcattagtaggaaatcaacgatttggtattgtatgtttccaagtgttattattaaaataccctactaggttcaaatatggtcttttggttttattagaatacgggagtaagtatttatttggttaattactacttagaatcaattataatcttcaaattgaattttttattgctgtcttccctattagaatctacttttgctttttttcaatccttaattatgttagagacctattcaaactcatatctccttgatttatataatgatttgacaaagtttgagggtatgttatatgtgcaggcactattgtgaacacagtgcgtgaagcaatacacaatacagatgatctctgttctcatggagcttacattttttttcccaagtgtttctccagcattgattggccttccctgtccagccaaatagcaggtcatcagcctagctgggactgtatgtccttgtagtgaaaaaaggctgtatcaggcttccaatgcttaatgaaaatagcctttcttttttcttttttcttttcttttctttctttttttttttttttttttttgagacggagtctcgctcttcttgcccaggctggagtgcaatggcacgatctcagctcactgcaacctccgcctcccgggttcaagcgattctcctgcctcagcctcccgattagctgggattacaggcatgtaccacgttccctggctaattttgtatatttagtagtagagatggggtttctctgtgttgatcaggctggtcttgaactcccaacctcaggtgatccacccgcctcggcctcccagagtgctgggattacaggtgtgagccacggcgcccagccatgagcttacattctaatagaaatgattctgtatttgattttgtattgtatttttatttctgagatgtagtgtgtggtttgaattatttaaatcagactagcagagagaaagtgggttggagtacatcaaactacaacattcttcctaaagctacatcagaaatggaggtatatttttaaaggataaaagataggcaagaatgtctttggggatagaaatgacatcaaatgaaataatgtatgggagagcaacttgtaaacagtgctttgagttatgtacttactcattaaggggcaggttgtggcataaaagcacaggaagttagtacttgtgtctttcatcccttccctagcttagtagttgtaccttattaccaaattcaggaaaatcgagtaactctaaaaaggtgaacatacacttgaggttgtctttgtcgtagctttttatgctaggctacaggaaaattatagcctttgtgcctctatttatacttgctatagtgccaagttgtaattcacacaaaagaatactagttaaacacttggaaattttccttccttggcaataagtttatactgtgttaatcaatgcattagtgttaaaatggacagtttccagaggcaccttttcacttctgcttgcttatttggctaggtatttaactagtccacacactgaacggaaaaaaatgaactaagagcctattagactgtttaaaaattgctatattatactgatcttaaaagacaaaaattgtttgatgtatttcataaaacacctagcacaatctttgggattttttaagtgttaaatttgattaggtttattttatttgtcagaaattctatgtatttatgatgtacaacatgatgttttgaagtatatatacattgcagaatggttaaatcccgctgatttaacatatgcattacctcaaaaagttagcattttgggagaaaacaacacttaaaatttactcttagcattttacaaagatacaatatagtgttaactgtagtcactgttatacaatcgatcttctgtatttattcctactacccaactgatactttgtattctttgaccaacatcttcccatcccacccctacccccagcacaatctttgaagtcttggtaactttactgaaggtattgaatgtttattgtagttgttgcctttttgaaagtacgtaaataacatctttgcattgtagaaaaagttcttctttatagataaacatgaagaaatgaagaaaattgaaatcattcataatcctgcggctttgagataactgatgagaacagttttagattttttaatttattctatctgttctttttacaggcaaacgaagatttaatattaaattgtggaagaccttcactgattgttttaactgtctgcctatagcagccattgtggatgagaagatcttctgttgtcatggaggtagactagtaaatttgccttacagattttttttttcttctattattcggaaaatacctataataatcaagaggctgtgggaaaacaaagcagtgcttgtatgaaaactcaagtgattaaaactgttttaaacatggctaaacctcttttaaaaattagaaatttatttataaatgaaaacgtaatgcttctttaaatacagtttcacatataaggagtgtaactagagttgtttgagaaagtctcctttgcctcaagttttaacagaaagacacccctgctatttattagtaaaaatgagaggatatcttcctgcctatattgaaatctttcatcagaatccaaataaaattgagtttcatattcacatatatcaagaattctaggcttcatataatatatttgctttagtattaacaataaaataataaaataatctttggcagattggtaggttactctgtatggcttatgaaataagcttgaagttttttgaaagtttatttgaaaaattaatgtatatgtctaacacctagctagtagatagtgtatgatctgaccatcttgaagagttaaaaacctgttgctaagatacatttatgaaacagataattgagtaataaaaggtatttaatatgtcagaacttattaagacactatgagaagtcaggaggaggaaagactgggtacatgtgtgtatggaggcatgggttttagaggataaataggctttggagaagttaaaaggacttgagacactctttgttgtctagtttagggaatgtgtaagaactggcctgactagaaggtcaagtacatgctgggaaatgatggaatcagattatggagatttgactattgagcaacggagtttgttgagtgatttaataaccgtattgggtttttgtacagggaagtaatttaatggaaatagtgtaagaaaattagtttttcatcaatatacgggaagaattaaagtggaaaaacgctgaaaagatagatgcacttgcctaggcccgaggtaataaatggtttattctctggcagaacataattcactatattaattgaaatatgttttccagtagacattaaacacgtaatgaattgtagttcttgtacattagtgtagctaaggatagaagtgtagcagtatggatacgaatgtttagcttcattccttgtgatgatacttggattgcctatcagagagaaataatagcttatataccacttcaaagattgcagagtatttttatgagcttttcctgttaatccttctgatcaatagtttatgacttcatgaagataatgaacatgttggagtgggatgtgaaaagaaggtcttagggaaccaagtgaggcactttcacttagaagttaaaatgtttaagattagtggtaagaactggcaaaagtggtaggaagggttgcagttgattatgtttctatgaaggccagggaatgattggaaaaaagaatggtagttttttttttccttgggaagtcttttgaaatataaaggaatttcatttccctggtagggaaaaatggaaaaaaacaattataatgctgtattatatcaaagtggaattcctaataactctgtgttgccctttaagacattaaaaggggcagggcgcggtggcccacgcctgtaatctcagcactttgggaggccaaggtggttggatcatgaggtcaagagatcgagaccagcctggccaacatggtgaaaccccgtctctactaaaaatacaaaaaattagctggatgtggtggcgcgcgtctgtagtcccagctactcgggagactgaggcaggagaatcgctcgaacccgggaggtggaggttgcagtgagctgagatcacgctactgcactgcagcctgggtgacagagtaagattctgtctcgaaaaaaaaaaaagacgttaaaaggaacacttttcagtatctttaatttcacatagtgaatctattttgattaaatgctttttattcggtgactgttaaaatattaaaatttttagctgttagtactatgtctcatctttttatttataggattgtcaccagacctgcaatctatggagcagattcggagaattatgagacctactgatgtccctgatacaggtaagtgtagagagaagtttaattgttttgtgtaaagtgttttcatatttgaacttgattacatttagtggaagtaggattggcttatgtaataaataaaagagcttttttcatagtcaataagccagctacagattactttatttgcagtaatacacaatttttcactttgcactgatcctcaaaatgcggaacctttttatttatagtcataatgtagttatttgatcacaaaaaagattttgctgttgagtccttatacctatttttctaaattccctcattcatactagcattggttatgttcttcaaaaatttgttaaattagacaaaagttttttaattaaaataatctattctttgcttcttaggcaaggatacaactaccttcttttcaaatgtctagtagttttttgttattttctatgtcctctatttgaaatttttagagctttttttcaagggggtgtgggagggatttcaatctgttgcccaggctggccttgaattcctaggctcaagcaatcctcctgcctcttctgagtagctaggggactccaggcatgtgccatacctggcttaaatttttagagctcttaaggctctagagacctgtggtcagttttgtgaaaattacattggaaatctctgggaaggctgggagcagtggcactcacgcctgtagtcccagcgctttgggaggctgaggtgggcaggatcacttgaggtcgggagttccagaccagcctggccaacgtgatgaaaccccatctctactaaaaataacaaaaattagctgggcatggtggtgcgttcctgtaatcctagctactcaagaggctgaggcacgagaattgcttgaacctgggagacagaggttgcagtgagccgagatcacaccactgcactccagcctgggtgacagagtgaaactgtctcaaaaaaaaaaaaaaaaagaaaaaagaaacaacaatcaccacaaaaaatgctgctcagaactacacatcaaccattataaatggtatagatcctctgatgtcagtagccatgtaatacactgtatgttgtaataaattgtgttaggagcttttttttttttttttttttttttttttgagacagagtcttgccctgtcaccaggctggggggcagtggtgcaacctcggctcactgcaagctctgcatcccgggttcaagtgattcttctgcctcagcctcccgagtagctgggactacaggcgcccgccaccacacccggccaattttttgtatttttagtagagatggggtttcaccatgttggccaggatggtctcgatctcttgatcttgtgatccacccaccttggcctcccaaagtgctgggattacaggcgtgagccaccacacctggcctgtgttatgagcttttttacataacacgacttgttgtgttgattattttttaaagtatgcattttatcacagtttttgattgtaattatagaatacatttgactaaacctaacaaactggtatttaaatagtattttatttaaaatgctgtttttaggcctggtgcactagttcatgcttgtaattccaacagtttgggaggccaaggcaggaagatcacttgaggtgaggagtcgagaccagcctgggcaacataacacccatctctacagagaaaaaaaaaatttttttttttaattagccacatgtcgcagcaccgcggctactcaggaggctgaggcaggaggatcacttcagcctgggaagttgaggctgcggtgacctgtgattgtgctcctgcattctagcctgggtgacacagtgagaccctctctttcaaaaaacacccaaagtgctatttttaaaataaggtataatataaatatgtattactggtcattcttcagaatatctgtttatagttcagatgtaaactgatctagtgtgatatgctaggcttctgtttttagcatgttttatttattttagcgtattttaatgagaagttttcaagatgctgaaaacagttattttgatctttgatagtgtctgcaacattcagaaatttaatttataagttaatacattatttgaagcagcctagttgattgtgatgctagctcattaaacaataaatcttgactctgaaattctttttgagacagaatttcactcttgtcgcccaggctggagtgcaatggcctgatctcggctcactgcaacctccacctcccaggttcaagcagttctcttgcttcagcctcccaagtagctagaataacaggttcctgccactgtgcccagctaatttttgtatgtttaatagagacagggtttcaccatgttggccaggctggtcttgaactcctgacctctggtgatccgcccacctcggcctcccaaagtgctgggattacaggcgtgagccaccatacctggcctgaaattcttatctagaccctgttaaggttaaagaattccagattgttgttgctaccattatttgatttttttaatgcatccagagtgtcatgtttcctaaacctcaaactctattctaattttatacagttttattgtctcactgaaattatagattcttgagatcgagaccatcctggctaacacggtgaaaccccatctctactaaaaattcgctgggtgtggcagtgggcgcctgtagtcccagctactcgggaggctgaggcaggagaatggcatgaacccgggaggtggagcttgcagtgagccgagatcgtgccactgcactccagcctgggtaacagagcgagactccgtctcaaaaaaaaaaaaaaaaaaaaagaaattatagattcttaataccttagctttctactggaaagaggctggaaagtatgaaagcattattaagcactgggtgggtgtatattttcgaagagtcagcttcaaccagaaagggcttaagaattgagactgactattccagtggattttgtgtgctagtgaatttcttaaaacaacatacagggagcttgggatatatcaatgatagagattgaatacattgccactaattctacaaatggttttataatgcctctatcaagatatgatgtactgtaactgaaaatgaagatgccctttcctcctaaaccatactatgatgtttatggattatttaaaattcttaaaactgacttaactctttaaaaattatcttcttggctgggcgcggtggctcacacttgtaatcccagcactctgggaggccgaagcgggcggatcacgaggttaggagatcgagaccatcctggctaacacagtgaaaccccatctctactaaaaatacaaaaaattagccgggcgtggtagcgggcacctgtagtcccagctactcgggaggctgaggcaggagaatggcgtgaacccgggaggcagagtttgcagtgagcagagattgcaccactgcatcccagcctgggcaacagagcgagactccgtctcaaaaaaatatatatgtatcttctcttagccactgcaattcttagaaaatcaccatcaccatctcaacatccacccttcttttaatatcactacagttggacttttacccactccgttaccctcaatcaccaagaaacttgcattgaggtctgatgatttaattgtaagtggcacatttttggtcttcgtcctactgcccttgttcatggcacatttcctactttaggacgattgtttcccctttcgggatacgtttactatactaagccctttgtcttgatcattccttctcaatacctaatgttcattctccctcatctacctgcctattaaatgtttgaattcctcagaattcatttgctggtcttctcactttgtattcctaaggtggtctgagtccctcttaagatttccactaccattaatatgatgacaatactcaagtaaatatttttaaccctgacctcttttccagggctccaaacattaccaaagtcaaggatctgatctgattttgttcttccacatttaaaaaaaaatcctcagggtctcagtagaaaatgtttttttttcgctcaagtgatctgtagatcaaatacaattccagtgaaatcacaattgagttttttatggaattaggcaattgtttttaatgtttatgtggaagaacaaaagatcaaaaagtaaccaagacaatcctgaaaaaaaaatggaggatactagccctaccaaatataaagacttattaaaaagctctagtctgttattggctcagggataagtaaaatagactaacagaatagactgtggtgctcagaaacagtggaaccgtaaggaaaggatgaactgttgaggaaataccactgggttgtttggttattcatatggagaaactacccaaaagatgaacagccttttccatgtattgttgtgctgtaagtgccatgcacatatttcctatatgtggaaagggttggaaagtactaccttaagaggccatggaagattatatttgtatggtttgtatggttgctttaacaaaagtataagttacataggctggaatacttaggcaagtgatggtcattgtttagtaaaaggtaagagggcaagaaaggaactgaaatttgatgtagatgtgctatattctataaatctgtaaattttgttcttaattgctgtatttctgtcctttaaaggtttgctctgtgatttgctatggtctgatccagataaggatgtgcaaggctggggagaaaatgatcgtggtgtttcctttacttttggagctgatgtagtcagtaaatttctgaatcgtcatgatttagatttgatttgtcgagctcatcaggtatgaaatataaaactcttaagctttttcttttttctttcttttttttgggggcagacggaggggcagacagattcttgttctgtcacccaggctggagtgcaatggcgcaatctctgctcactgcaacctgtgcctcccgggttcaggcagttcttgtgcctcagcctcccaagtagctgggacgatagccgtgtgccaccacacctggactcttaaacatttattatttgttaacttcacataagccctctcattttgttttgaggaaacatatccaagaaagctaaatgagttgtacaaggaaagcagtaggagaggtggcagagccagttcctggatctcacacttaacaatctagtgatttgttaaccttttatcaccctaaaagaggaaatgtttagaaatattttggggctgggcatggtggctcacatctataatcccagcactttggcaagctgagatgggtggattgcttaagtacaggagtttgagaccagcctgggcaacaaaatgagattccatctctacaaaaaaaatcaaaaatttagctgggtgtggtggtgcgtgcctgtggtcccagctacgtgggaggctgaggtgggaggatcgcttgagcctaggagatcaaggctgcagtgagccatgtttatgtcactgctctccagcccttgtgaaagagctagacccagtctcaaaaaaaagaatgtatttttgtagtatacagcaaacctaacaaaattttcagtgcttctttgaagctttgtagatatgatttagatttttttttttttttttttttttgagatggagtctcactctgtcacccaggctggagtgcagtggcacaatctctgctcactgtaacctccgcctcccaggctcaagcgattcttctgcctcagcctccggagcagctgggattacaggcacgcaccaccacgcctggctcatttttgtatttttagtagagacagggtttcaccatgttggccaggctgcccttgaactcctgagctcaagtgatctgcgcctcagcctcccaaagtgctgagatgacaggcgtgagcgtgaaccactgtgcccagcctcatttttatataatctttgtgcataatggcgattttgataagagcaaaccacactttaaaaaagtatttttgagaccattgaagaatttgattgtggctcagtgcagtggctcatgtctgtaattccagcactttgggaggctgaggcaggcagatcctttgagccccaggagctggagaccaggctgggcaacatgacaaaactctgtctccacagaagatacaaaacagccagatgtggtggtgtgcacctgtagtcccagcttgcttgggaggctgtggcaggaaagtcgcttgagctcaggaggcagaggttgcagtgagtgtagatcgtaccatttcactcctgcctgtgcaatgggtgtgaaactcagtctcaaaaaaagagaacatttgattgtagacttggtaataggtatgaattatttaatcttttaattttttgtttttgtttgaggccgagtctcactctgtcacctaggctggagtgcagtggcatgatctcggctcactgcaacttccgcctcccaggttcaagtgattctcctgcctcagcctcccgagtagctgggactacaggcgcctgccaccacgcctggctaatttttgtatttttagtagagacggggtttcactatactggccaggctgatctcaaactcctgaccccaggtgatctgcccacctcagcctcccaaagtgctgggattacaggtgtgagccactgctcctggccggtatcaattattttagctgtttcataatcattaattgttatgtaagaagaatgtctgtgtttttccaggaatgcatacttgagtgtgtagaggtaaaatgacatgcctgggatttgctttaaaataatatcagcaaagagaaaaggagagggagaagataaacaaatgtagcaaaatcatgaaaagtattgaattaggctgatgagttaatgggagttaattcaagtcgtctttatctacttttgtgtatatttgaaatttttcattataaaaacatttatacagttcagggtttttggttgtcttttatgtccgccaccactaactagattgtgtactctaaggaagggattacaacttgtttttgaatctctgttgctttatatagttgctgacattgtagcatagtgtatcatgcacatacagtgttgaataaattaataaaagcgtatttaattaagtatatgctgtcccacagtacttccctttctttctggagctttactccccatgacacttactactgctgcctctttctttgatcctaagtgataaaaatgactggctttttattctttgcttattgtatattacatgccacttttatttagcaggatttcttggtggttccattgaaatgaggtcatgttcccacatcctttgattttggcaaagtagttttttgttgttgttgtttgttttttttgtgagacagagtctcacactgtttcccgggctggtgtgcagtggcaccatctcggctcgctgcaacctccacctcccgggttcaagtgattctcctgccttagcctcccgagtagctgggactacaggtgtatgccactagcctggctagtttttgtatttttagtagagacggggtttcactatgttggcctggctggtctcgaactcctgacctcgtgatctgcccgcctcggcctcccaaagtgctgggattataggcacgagccacctcgcccggcctgacaaatagttttttaaatcagtaaaactgtgtttatgattttgataaatatgccttctattaaataacaaggcagaattgctaagtttcctaactaaaatgacccctgataatcagtttttttcagccatagatcttcattcactggtcttttcaaaaataaatgatgatttttaaaaatcaggtttctgaactacgctagtttgcatggtcttttgttttccagaaactgccaaaatccttctattgtgcttattttcaaataattgtctctaaaattctagaaatattccctttaaaaaagaaaatttgaaagaatgaacaagtaagcaaactaaaaaaccaagaactaaaaacatggagtccatgaccagcctgggcaacacagtgaaacacgaaaccctgtctctacaaaaaaatacaaaaaactggctgggcaccgtggctcacgcctgaagtcccagcattttgggaggctgaggcgagtggatcacctgaggtcaggagttcgaggccagcctgaaaaacatggagaaaccccatctctactaaaaatataaaattagctgggcacggtggcacatgcctgtaatcccagctacatgggaggctgaggcaggagaatcgcttgaacctgggaggcagaggttgccgtgagccgagatcgcgccgttgcactccagcctgggcaacaagagcaaaactccatctcgaaaaaaaaaaagaaaagaaatacaaaaaaattagccaggcatggtggcacacacctgtggtctcagctactcaggaggctgaggtgggaagatctcttgagccccaggaagttgtgactgcagtaagccaagattgcaccactacactccagcctaggcagcagagcgagaccctgtctccacacaagcacatacacagagaaacaactaaaaacatgagtgccatttgctatgtggagcaggggtcacagggattagataaggtggctatggtcttaggagaaaaggttcatctaaattgtaccataaggtaatttcagcagaagcatgttcctagtactgaggtaggagttattttaataagtaaatgccttcgtaattgcttttttatattagcaagtggtgtcggataattttgctaattaaaagatacctaaaataaatttaaaagtaatcttgcccaccgtaaataaatataaaatatttacatattattgtgttcctcaaatttgtagactgagagaaaaattgtttaaattactcagaaattgacaactggttctagaaatgttaactcatcaaggccaggcacggtggctcacgcctgtaatcccagcactttgggaggccgaggcgggcagatgacctgaggtcaggagtttgagatcagcctggccaacatgacaaaacctcatctctactaaaaatacaaaaattcatcgggcttgctggtgggtgcgtgtagtcccagctatttgggaggctgaggcaggagaatggcttgatctgggaggcagaggttgtagtgagccgagatcatgccactgcactccagcctgggttgacagagcaatattccatctcaaaaaataaaaaataaaaataaaaatacagtctgttggcagttaatttaaggagcatgatccatgtaaattaaactcaacttagtaatgttttcagtgtgtagccagtagtgttttcagtgtgtagccatgtaataataatacatattttaaaatcaaagcaagaagtaagctcttcgcaaagtcgttcattgtctggttgttgctcatttaattaaggattctgtagttggattataatgccctcatcaaagtaataattgatgtccctctgaaaaacacgtagattaattaaaataaaattaaatttaaaacttaaaaacaaaaaaagtacttagtgtcccagctccctgtagtcatgatgcctattagtatgttttgtttcatggggagccttgcaaatggcatgatttttaagctttcaggagtggtgaatttattattccaattaagctaaattgtttccattatagaaaaggcacaccagttttaatgttctaagcaaagtttaagaataaaaatcacaacttccaaaacaaaacagggtggtttgggggtgaggtggggcacagtctttgccaccttaaccttaattagtatagatggttcagaattacccaccaataaatgttttttcttctgacatttcctttgacaggtggtggaagatggatatgaattttttgctaaacgacagttggtaaccttattttcagccccaaattactgtggcgagtttgataatgctggtggaatgatgagtgtggatgaaactttgatgtgttcatttcaggtatgatgtaaacataaatatataagaactagaaatctaataaaaactattatcagaattcgttttagatttgtgaagtttccttgagcaagtgttgaaagtctgtttaattcagaagtgattaccactcaaactcattagatatttctattttcagtcacaaagtttccagcatttgtggtatatatcaaatatgaccgttttagggaacctaagttacgtcagtcattcttgattcagatgttttaattgttaaaattcctctatgaccttttgaatagtggcttactaaaagaatgtgaatgtaatgaatagtttaggcttttgttttcttttgcaaagtaaatgggtggaacagacactgggaaaagaattacaaactcagatgaaaataaagtaaaatagttccctggctgggcacggtggctcactcctgtaatcccagcacttcgggaggctgaggtgggcggatcatgaggtcaggtgatcaagaccgtcctggctaacactgtgaaaccccatctctactaaaaatacaaaaaaattagccgggcgtggtagcatgcacctatagtcccagctacttgggaggctgaggcaggagaatgtcgtgaacccgggaggcagaggttgcagtgagccgagattgcaccactgcacgctagcctgggcgacagagcgagacttggcccccaaaaaaaaatagtttcctgaggaaaaaaaactttcaaaataatactaaactacccgtttatgtcacaggaacatttatttatttatttttttcttttgaggaagcacattgtataccatgtacggggggttgtttgctcacttttattataatatttttattaatttatgtacgtactaggttcaggggttacatgtgcaggtttgttccatgtgtaaattacatattgctgaggtttggtgtatgaatgatcttatctcccaggtagtgagcatagagtttttcatttcacattccccctcaccctttccactctagtagtccccagtgtctattgttgccatctttatgtccatgtttacccagtgtttagctcctgcttatgagtgagaatatgtggtatttgattttctcttcctgcattaattggcttaggataaaggcctccagctgcatccatgttgctgcaaaggacctgatttccttccttaattttatggtcgcatagtattccatggtgtatatgtaccacattttctttatccagttcactgttgatggacatctaggttgattacatgtctttgctattgtgaatagtgctgtgattaacatacaagtgcttgtctttttttgtacatacccaaagtagtgggattgctgggtcgaatggtggttgttttaagttctttgagaaatctacacactgctttccacggtggctgaactaatttacactcccaccagcaatgtttgaacattcctttttctccacaaccttgctgacatctattatcttttaactttttattaatggccattctgactggtgtgagacagtatcttgtggtttgatttacattaatagtgatgatgagcattttttcatatatttgttggctgcacgtttgttttttgagaagtgtctgttcatgtcctttgcccatttttaatggggttgttttttgattgttgaattaaatgccttatagagtctggatattagacctttgttggatgcatagtttgtattttctctcattctataggttgtctgtttattctgttgatagtttattttgctgtgcagaagctctttagtttattttggtcccacttgtcaatcattgttttggttgcagttgcttttggggacttggtcataaattatttcccaaggacagtgtccagaatggtatttcctaggttttcttctagggtttttatagtgtgaggtcttatatttaaatctttaatccatcttgagttaatttttgtgtatggtgaaaggggtccagtttcagtcttctgcatatggctagccagttatcttagcaccatttatcaaataggcagtctcttctccattgcttgttattgttgactttgttcaagatcagatggttgcaggagtacagctttatttctggctcctccatcctgttccattggtttatatgtctgttttggtaccagtaccatgctgttttggttactgtaagccttataatgtaccttgaagtttggtagtgtgacgcttctggctttgttctttttgcttaggatgctttggctattcgggctctttttcaatttcatatgaattttagaaaagttcttttctaatgctgtgaaaaatgacattggtagtttgataagaagagcattgaatctgtaaactactttgggcagtatggagattttaacaatattgattcttctatccatgagcatggaatgtttttccatttgtttgcatcacctctgatttctttcagcagtgctttgtagttgtagagatctttcacctccctggttagctgtattccttgatattctttttgtggctattgtaaatgcagttcttgatttggctctcagcttggatgtttttgatgtatagaaatgctactggtttttatacattgattttgtatcctgaaactgattaacaaagttgttcatcagttccaggcacgttttggcggagtctatggggttttctgtgtatagaatcattatcgtcagtaaagattgtttatcttcctctcttcttatttggatgccttttatttctttatcttgcctgattactctggcagggactttcagtactctgttgaataggggtggtgagaatggtcatcctagtctcttgttccagttctcaaggggaatgcttccagcatttcccttttcagtacgatgttggctgtgggtttgtcagatggctcttactattttgaggtatgttcctaatttttcagtgcctaatttgttgagggtttttaacatgaaagggtgttgaattgtattgaaagcctttctgcatctattgagatgatcatatggtgtttgtttttaattttatgaggtgaatcacatttattgatttgcatatgttgaaaccttgcaccccaggaataaacccttcttgattgtggtgagttaactttttgatgtgttgctggattcggttttagtattttgttgacgattttgcatctgtgttcatcagggatattggcctgaggttttcttttttccttgtgcctctgctaggttttggtattacaatgatgctcacttcgtagactgagttaggggggaggtcctcttcctggattgtttggaatagtttcagtaggattggtactagctctttgtacatctgatataattcagctgtgaatctttctgggccagggctttttttggtttataggtttttttattactgattcagttttggaacttgttattgttctgttcaggattttaatttcctcctggttcagtcttcggagtttgtgtgtgtttctatgaatttattcatttcttaggaggtcatgtgtgtttctatgaattcatttcttgggagcgcatgtgtgtttttgtgaatgtattcatttattctaggtttcctagtttgtgtgcacaggtgtttataatctctaggtattttgtatgtctgtggggttggttgtaatgtcacctttctcatttcagattgtgcttatttggatcttttcttaatctagctagcagtctatcttgtttattcttttgaagaaccaacttttggtttcaacatttacatttttaaaagaggccagttttagctttagtgtgctcctacaaatcattcttgttcactgagtaaaacagcttcagtactttgcttttgcaaaggctattctatgtctttgatgtacacacactgtatttctcttaaaatgcattggggctttcccattcctctttctctttatcataatatactgtttccatcaattagtacaattgaatggtaaatggttgattgctcatttacaaattaaatatagtaaccataccttatccttccagcagttgtcatagtggtgttggattaatatattctcttcactaaggcactattatgttctaaatagtttttaaaaaatactttgtacatatttctatactgttttagttttggaaaatcatgacttttttatgtataatgtaggttaaagctctatttacatccaccagaatgagtgtagtaaaaaagccagactgtatcaagttttggcaaggatttggaggcattagaatcttcatacattaatggtgggaatagagttctcatatgatgcagcagttttacctctaggtatatacccaagatatatgaaaacatgttcacacaaaaactgttacataaatgtgcattattccttatagccgaaaaagtcacacaattcagatgtccatcagctactgaatagataaactgtggtacatccatacaatggactgtacaatgtacaactcagcaataaaaaggaatgaaggactgatacttcaaaactggttttgaaaacctgagtgaaagaagccagtcacaaaagaccacatattatatgatgccagttatatgaaatgttcagaataggcaattctatagagacacatagtagattagtagttggttagagttagaggatgtggagagcaggaatggggagtgactgctgatgggaacaagctttctttttggggtgacaaatgttctaaaattagatttatatcaataaagctgttttttaagtaaaaagatctatttatacataaataaataaggtctgaagggtttgtgacttgtaggttagttaccctcctaagttactgtatcagaattatgatgacaacatagatcttctgactcttaaaccagtgttcttttcctcacaacagagatacactaagttcaaaaagttttttacttaaatctaaggattggatagttgatagagttagtgtacttttgttattaaaatatgagtggaatgacaaagttcagacatttgcacactttaagaaatgtttatggaactttgctattctacatttttattgcatttttgcagtttatgccatttattgaaaatcacaattgtaacaattttcttaacttctgtacatgaaaaaataacattatttgttaataatttactttaagatattgaaaccatctgaaaagaaagctaaataccagtatggtggactgaattctggacgtcctgtcactccacctcgaacagctaatccgccgaagaaaaggtgaagaaaggaattctgtaaagaaaccatcagatttgttaaggacatacttcataatatataagtgtgcactgtaaaaccatccagccatttgacaccctttatgatgtcacacctttaacttaaggagacgggtaaaggatcttaaatttttttctaatagaaagatgtgctacactgtattgtaataagtatactctgttatagtcaacaaagttaaatccaaattcaaaattatccattaaagttacatcttcatgtatcacaatttttaaagttgaaaagcatcccagttaaactagatgtgatagttaaaccagatgaaagcatgatgatccatctgtgtaatgtggttttagtgttgcttggttgtttaattattttgagcttgttttgtttttgtttgttttcactagaataatggcaaatacttctaatttttttccctaaacatttttaaaagtgaaatatgggaagagctttacagacattcaccaactattattttcccttgtttatctacttagatatctgtttaatcttactaagaaaactttcgcctcattacattaaaaaggaattttagagattgattgttttaaaaaaaaatacgcacattgtccaatccagtgattttaatcatacagtttgactgggcaaactttacagctgatagtgaatattttgctttatacaggaattgacactgatttggatttgtgcactctaatttttaacttattgatgctctattgtgcagtagcatttcatttaagataaggctcatatagtattacccaactagttggtaatgtgattatgtggtaccttggctttaggttttcattcgcacggaacaccttttggcatgcttaacttcctggtaacaccttcacctgcattggttttctttttcttttttctttctttttttttttttttttttttttgagttgttgtttgtttttagatccacagtacatgagaatccttttttgacaagccttggaaagctgacactgtctctttttcctccctctatacgaaggatgtatttaaatgaatgctggtcagtgggacattttgtcaactatgggtattgggtgcttaactgtctaatattgccatgtgaatgttgtatacgattgtaaggcttatgtcactaaagatttttattctgattttttcataatcaaaggtcatatgatactgtatagacaagctttgtagtgaagtatagtagcaataatttctgtacctgatcaagtttattgcagcctttcttttcctatttcttttttttaagggttagtattaacaaatggcaatgagtagaaaagttaacatgaagattttagaaggagagaacttacaggacacagatttgtgattctttgactgtgacactattggatgtgattctaaaagcttttattgagcattgtcaaatttgtaagcttcatagggatggacatcatatctataatgcccttctatatgtgctaccatagatgtgacatttttgaccttaatatcgtctttgaaaatgttaaattgagaaacctgttaacttacattttatgaattggcacattgtattacttactgcaagagatatttcattttcagcacagtgcaaaagttctttaaaatgcatatgtctttttttctaattccgttttgttttaaagcacattttaaatgtagttttctcatttagtaaaagttgtctaattgatatgaagcctgactgattttttttttccttacagtgagacatttaagcacacattttattcacatagatactatgtccttgacatattgaaatgattcttttctgaaagtattcatgatctgcatatgatgtattaggttaggtcacaaaggttttatctgaggtgatttaaataacttcctgattggagtgtgtaagctgagcgatttctaataaaattttagttgtacacttttagtagtcatagtgaagcaggtctagaaaataagcctttggcagggaaaaagggcaatgttgattaatctcagtattaaaccacattaatctgtatcccattgtctggcttttgtaaattcatccaggtcaagactaagtatgttggttaataggaatccttttttttttttttaaagactaaatgtgaaaaaataatcactacttaagctaattaatattggtcattaaatttaaaggatggaaatttatcatgtttaaaaattattcaagcactcttaaaaccacttaaacagcctccagtcataaaaatgtgttctttacaaatatttgcttggcaacacgacttgaaataaataaaactttgtttcttaggagaaaatgattctgtaattccagtgtcactaatttatattgttctttcctctgatttttttcaggttagtgatttttttgtatacaatttaatccaaatgttatgacattcagaaatcatgaaacacagtagatatctgttataatgtggtgtatcacatggattataaagcaaagttatggtcgatttctattcttgaaagaatcaactacagtgaatcctttgcatttgaagccttaacatgcattgctttaattttgcccagggacaaattttaataatcagcaagactggtttgtgcaaagcgttgagtcatcaggtatttagagcctagccagctacccagtatccatgctgccatatcccttcattgtaaaaagtacctaaacattcgtgaaatgattttttttagctgaaaaatgctggcaagaagaattttaaagcttaaaataggtggtaaatttgaagtatgagtgtgttcacgagaaacataggcttttcaaaaaaatttttattcaaggcaaagcaaggaacatcttgagatatgtctcaagaatataaagatgtattattttaagccaaggagctgaaatatatctcagtttataaattcaggtatattctttttgtctccatggcaaccataacttttgaaccaaaaaaaattgtttttacatctttatgctgaaaatgtgtttagattaggaatatggtcgggctgaatttgctgttgctccctaaccaaatccacctcttgttttccttgtgagtccatggctaaatcaaagctgcccctgagaagagacttaatccaagcctgattgtactagtggcatcacttagaagtaggctttccctcttcctagtagatctcaatgttttataattccttaaaacagctgaaaattgggacaacatactttacgcaatgaacagtagttaaataggaaataaactagttccatataagtatacacctagagttttaattacctttataatgtttcttaaaagtgaaacttagatacaattgtgattggatacttagatactaagtgaaacttagtgtaacaattttgatctgttaaattggattttacatgtacatttgaatgccagaatttctaaataaatcccctggttaggaaattttaaaagtcaaagcttgttttcttcaaccactaccttctacattggttgacttagaccgtaagctttttaagtttctcattgtaatttaccttctcatgcagattgctgatgttttattaaaccttatttttacaaaaatga

SEQ ID NO: 9 - DNA, 34546 bp, Homo sapiens
aaggagtcggcggccattttgttcttctcgtggttccagtggggagagaaggaggaagtagggagcggggtggcaggggggggacccgccgcggctgctgccaccgccgccaccaccgcctctgctcgtggcgtgggaaaggaggtgtgagtcccgggcgcgagccggcggcggcgccgctgcgggagggtcggcggtgggaaggcgatggcggatttagataaactcaacatcgacagcattatccaacggctgctggaaggtaaggggggcggcgggcggctgaagggggaggcgggcctgagggcggcaggcggccccgggggctcgagggaggggagttgggaggccggttgggcttgcagcaccgtggggagtgagctcctcgagttggcccacgggccggggactgcagccggagggaggccagcccgggattcccaacttgtcgccagctgcggaccctccaggctccgctcattcattgcacgaggatgcaggcttcctcccggccgtggaatttgaaaactccgctccccacccccacgtttcccgcccctacccgtttgcccccgcgcactggacgggtctttggagcgatggttggggatcccagtccagcctcctggacgacgtgtgccgtgctgtgcgagatggacttttcggtgctcgtcctctcggaaggcagtttgatgagggcatgagttccgccgggtttgggggctcttttcctgcttgcttttatttttatttttcgtggttctggggtcttggggggcccatttcttgttacttggtaacaagcgttcatgtctcaagaggtgtgagaacctgtcttcgcctgccctccaaatccttttaagttgttctaatcttggagaggtctccgcgctcttttcctgagcgtgcatctgaagaggaagccaacgaggctgacgctatcattatctttccaatgtgccaggctctgtcttaagcactttgcgtccatgaattattactgtaatcataactgacgcttctaaagcgcttactgtgtgtcaggcattctttgagcacttaagacagatttcatttaattctcccacccattgcaagatatttgcaattttttttgtttttgttattttacagaagaaactgagatgaagtcacttaaccaaggtcacacagttagtgacagttggatttcaaatccacatagccgggagctagaaccctctacagacaacactgtcaccttatgtaggaaggaaggtggccagtggttaagtaaaggttaatagaaagctttaaatttcggctcatctaagacttgtgaggtgcctggcaattgattcatgttgatgtgggagaggggcgaggttcagcttttaaagacggaagctgaaatgagctgctttcagattcaagggcatgtcaacggatgctaacatcttatccaagactttgtaagaattcagtcatatcccctaacttggacacttcataatgggaacttggcagtttcaactgctttttgcctttatacagtcctaaaatattgttggactataaatgctgtgagtggtggagtcgagggcatgaacagttggacagctctggtgttagaatttacagatcttttaaagtaagttattaaaatgaagctcctgaaaatatctcgtccttttcttaatctgtgcattctttccttttatgctgtctttaaaaaaaaaacaacaaaaaaaacactaaatgtcattaaggacccctgaaatgctcttgaaatgttattgcaggtgaaaaccttgtattgtaggtgaaaaccttgtcattcttttaatgccaggtatttatataagaaccatactttttcatactagttaatatagttctttaatttattcgaggcagcagttggtcagaaactgattaaaatagtttgctgtaagaaaggaactttttgttaaatattttctttctaaattttaaaattctgcttatctctgcagggatatgtgtggctttatttctctgaggaaacaggaaaagttggaatttgcatggattagaaacattagaattttaagatcatctaaggcttgctaatttaagttctggtttgcccataactgcctctattcataaacagcttttgttaattttgatctttaaaattgtaataagtactttgcctggtaagtaatgatacatctaaaatgagatcattgtttcttaatagttcctcttgttatgtggcacttgaaaaccaggttttcttatatttcattttttgctgaaattaataaagcagtgcattttaatgccttgggaataggctaactgcaaagtatacttaaatttctttttttctcgtgggtgttaaattggcagagaaacagttgaagatgatccattgaggatatgtagatatctaagttaaatgatgttggtaataaattctgttttacaattttttatttaaaaaaattaaatttaataatttaaaatagaaatgagagtctcactgtgttgcccaggctgttctcaaactcctggtttcaagcagtcctcgcacctcgtcctccccaggtgctggggttatagtcatgagccaccgtgcccggcctaaattttattcttttcataatgcccccaaaaccttttttttccctaaataagatgtaagtatatctgaaagtagagggaatatgacttgtgactttggccagatttttttacgtgtttagaattgatcttttaagcttgcgagcagtataacctagttgagaagtagctgagtaatagtactaaaaagctaactgaaaagtgaagtcttaagaaaaacagcatgttggtttagatcttagtcactaacctttgcagttcttttcttctctgaactctgagtatttctgtaagcgaggcatcacctctgcaacccaccatggatgcttttatccaagctctgccttgctgatgataaaacttaaggttgttaactccttagtataactgatgttagttatttaaataactacacagtaaaacggcattttgagagaaaataagccagctgttgatttggtctgatgtattgaagacaagtgagatttggggcagttttactttgtaaatgacctccttcaaggagccatggtggctagcattttttttgcatagcaggtggttgtaacttacttcacatcttggtttgcaagaagcatctttgctttgaagaaggccattgtcaaagtgcaagaccatgtcaggctctgcagaattgcctgacttgttttagtgatttaaatatgatctctgccctggctcacagaaagtgcttacagatttaatcctctgatacataagggttgttctgaaggtagggagtgttcagatattctgaaactctaagattgtttaaataacccatatgttcatctttttgatgcagtataaaacgttcatattctggtgtttagccatccctggtcctaaagataataaaggacaaacataatggtttaaaaaggggaaggtttcttaatatgctaaccaaaaaaaaaaacaaaagtaatctttcctaatgaacacctgtaattatttatttttttattttattttggagacagagtctcactctgtcacccaggctggagtgcagtggtgggatcctggctcactgcaacctctgcctcccgggttcaagcgattttggtgtctcagccccctgagtagctgggattaacaggcatgcaccaccacgctcggccaagtttttgtatttttagtagagatgaggccaggctggttttgaactcgtgacctcaagtggtctgcccgcctcagactcccaaagtgctgggattacaggcatgagccaccgtgcccagcctgtaattatttgtttacagagcaggtaatttttacactcacatccaagttgtcagtttctaattccctcattgataagaactctgatgtgagcccttgcccttcctttcccatcagtatgttggtcatatgcccacttcctctcctcctaggcaaggtgggaaagctctgttggagacagatgatctgcagcttgcaggagtccttttttaaaccacagcattgaaaaaggattactctccttgtgcccgcagtgccctcttgcctgtcagtagcaggaggcagcagtgaaatctgacagaattgtgtgtgacgttgtgcttgcgggggatgtgacacacaggagcgctctgcattctgagcctagggtgttcctcctcataactgggcaaatggacatgcagttgtagcccagcagtggtcagctgtagaaattagtgaggtgatttgatgtctttgaaagcacagtcctattacaacctgtaacctgttgtgcagaatcacagtaccagtcagcccacttgactaggtgaggattcctgatggtgaagcagggaaattattgatgttatgtggacttcaccatttaacttaatttacaattgaggccattagttgacctgagagattaaaatatgaaggagataatattctgggtgtcagtcgctttgtagataaagaactggaaagtcattcaagaccaagaaggaaggatttcatttccatggagcttatggaattccaggatagagtttgatattttaagatcatacctctgtatccaaagcatcaagaacagatgatgtgtttgtggatcttggtgatttgtctgaaatctccagcttccccacttcactggggtttactgtcatccctcttctagaaggtagtggtcatcaccgacagtaaagcttacagcaaatgaaaactcaaaatgtagtctactcagtggtatattaccgaaatgtgttcttagactgtgaaaattaaagtttttaatggattggctttactcgaggtgtagaaaactcaagatgctttattgagaacagtagacagtgttttgcagccacatcctatctgccttgaaaaaaatggtgtcacttttattttttttactagagactgggtctcactctgtccccaggctggagacctgaactcctgggttcaagcgattcttcctcctcatcctcttggctagccggatctataggcacacaccaccacacctggctaatttttttttctttttctttttttcttaaagacaggggtgttgcctaggctgttctttaactcctggccacaagcagtcctcccacctcagcctcccaaagtgctgggattacaagcgtgaaccacagctccctgccttggtgtcacttctcaatgcctgaagtgtaaccccatctgttaggaaaatcaggatgggagagggcagggtgtggaaacaattgtgataatcagaacatggaagtctttctgctggtgactttgcagagaaacttaaagggttcttttttcttttcttttcttttttttttttttttttttttttgagtctttctttgtcacccaggctggagtgcagtggcaggatctcggcccactgctgcctccaccacccaggttcaagcgatttctcctgcctcagcctcagttgttgagtagctgggactacaggcatgtaccaccatgccctgctaatttttgtatttttggtagagatggggttttaccatgttggccaggctggtctcaaactcctgacctcagctgatccacccaccttggcctcccaaagtgctgggattacaggcgttagacaccgcatccggcccaaaggattcttgataacatttttgtggtttatactttttgcagaaaagaactcagtcgtttatgggaatgcttaatgttggaagtatttactgttttttgttttggtttgagtcttattttcctaaatctgtcaaatatcatgaggctattgatttcactgaaatgaatatgatgtggcatattgttaaaattatcacctactttttaacaggattattttaaaacagcaaaatcttcgttggcttatttttataccacttgtgcctttcttaggttctgaaacttcattagaattcagctagggcttttttagtatatgctatttctttaatatgtttttatttaaacaattaaggggcctttatgtggcaaggaaaatacttttttattccttataaataatctcaaacttgtcttaaatttccttccatagtttccatctttgctttgctttttgccagcagctaactttcaaaagttagtattgcttatgagaaagagctggtgttgtaaataatcaccaatgctattttatttatttatattttttgagacaggatctcactctgtggcccaggctggagtgcagtggcatgatctcggctcaatgcagcatctgtctcccgggctcaagtgatcctcccatcttagcctcccgagtagctgggactacaggcgcacgccaccatgtccagctatattttgtatttttggtagagactcaatttcaccatgttgcccaggctagtctctaactcctgagctcaagtgatcctcccacctcggcctcccaaagtgctgggattataggcccaagccaccacatctggctgctattcttttataattcaaatttttaccagtttgttttcaataaaaatgtgtggaagtttttattattgctcgtgtcagtgatttgccagaaaaatagatcagatacatttctacagagcaaccttcgcttttaacagtaataatacgttttcctgaatctccctttctagccagataaagaaatattaatacttctactgaaaagtcacatccttaggtctaagttggtgttgcttttttgttgttgtttgtttgtttgttttgagacggagtttgtttgtttgtttgtttgttttgagacaaagtcttattcttgttccccaggctggagtgtgatgatgcgatctgggctcactgcaacctgccccgtgggttccagcgattctcctgcctcagcctcccgagtagctgggattacaggtgcctgccaccacgcctggctaatttttgtatttttagtagagacggggtttcaccatgttggccaggctggtctcgaactccttacctcaggtgatccgcccgcttcggactcctaaagtgctgggattactggcatgagccaccgtgcctggccgagacggagtttctttcttgttgcccaggatggagtgcagtggcacaatctcggctcactgcaacttccacctcccaggttcaagtgattctcgtgcctcagcctcctgtgtagctggggttacaggcgtgcaccaccatgcccagcaaatttttgtatttttagtagagatggggtttcaccatgttggccaggctggtctcgaactcctaacctcaggtcatccacccatctcggcctcccaaagtgttgggattacaggcatgagccaccgtgcccaaccttctcctttggttttgaagttcaatgtagaacagattcttaattgggaattaagaatgcaccactagatgtagcatcgcggcctcgtgtccttaagctctgtgaccttggactaagtcactgtaaaatgggagatgacagtgcctgacttcctaggatcgttgacaggattgaatggcaatataaagcccttagtacagtgcctagtggataagtgctcagtaaatgttaactacaacgagtaaataattctgtttgatggacattgaaaatagtgaagtgggccaggcacggtggcgcacgcctataatcccagctacttgggaggctgaggcaggagaatcacttgaacccaggaggtggaggttgcagtgagccacgatcgcatcactgcactccagcctggcaacagagtgagactccatctcaaaaaaagaaagaaaaaaatagtgaagtgaatttgtatgcttttggctgatatggcaactttttttttttttttttttttcttgagatggagtctcactctgtcgcccaggctggagtgcagttgcacgatctcagctcactgcaacctccacctcccgggttcaagcgattcttctgcctcagcctccgcagtaaccagccaccacgcccagcctgatatgacaacttttaagaactctccagtgttcacttggactgccaagccccatttgaaatctctgttagacaattcaacgtgaaatttaagagtaattctagattttcatactttgaaaaatactccagtaaagcctggtaagcaagtggccctattgttttataattttatagtgcagaaacaggctttttgctcatcaaaagtatatagtctgggctgggcacggtggctcacacctgtaatcctggcactttgggcgccgaggcagtggatcacctgaggtcgggagttcgagaccagcctgaccaatatggagaaaccccatctgtactaaaagtataaaattagctggatgtggtggcgcatgcctgtaatcccaggtactcgggaggctgaggcaggataattgcttgaacccgggagacggaggttgcagtgagccgagatcgtgccattgcactccagcctgggcaacaagagtgaaactccgtctctaaataaataaatatatagtctggaatatatgtttaattctaatgccaccagtcaacactacttaaagacttatccagatttcagtcatgcaacaaattttgagcacctgctgtgtgctggctatggctggtgtacactacagagcaggactgctgctgtgggtttacattctaccagtgggtccagatgagccggcagaagcaaactggcaagtataaaacaaatacaagcatggttgagggggtgtggtgggtagtgacctgatgttgagagagcctttcaaaaagcatgcagcaagcttgtgaggggagaggagatggatgagagaagcttttctccagggtgaagtgtggtatctgagcagaggcctgaagtctgagcagttgtatgataactgatggaagatggttttggcagtttctatctttatataacttttttctttctgctaattttagggaatttaagaagctttcagaccatgtttgttgatgtttttcataagcaccattctttaaatacaaatctttattttctctggaaaagctgcatctaatcttaatattaagagtaatttttaaggccaggtgcagtggctcatgcctgtaatcccggcacttggggggccagagcaagaggatcacttgagcctggaaggttgaggctgcagtaagccatgatcatgccactgcactccagcctgggcaccaggcttcttcaaaaaagaagaatttttaaattgttgttagtattgcttttgctttttactgatctctgaatgtccccacccagaggacaaacagaaacagaataaaggagttaaaacaaatataaactatgttcaaaggtgacagtggagagcgtccaaagccaaaaaaaaaaattgttagcaacattgatttcatctgatggcattgactaactgcaatagcattcataaaacaaagcttgccattatatggtatgagctatttatcgcagaaatactctcctagaagacttagagtcagctattcattcataagtgtgggactctgctgaaaaaaattcacgttgaaaccagaagcttctaggatttccttcctagccaaaaataaagcttcctgctgggcagtctgcccaagtttttggttaacttatggtgaaatcactaatgattggcattttctggccaatgaagtggggatcccatctttcaccgggagtatttcctgagttctaactctgtatgtgtgtctagagagaaaaaaaattacttacgaaaaaaacatttttgttgctcagttgctgttttattaaatatgcatctgtttcatatctttcctcgaagactattattactatgcttttattgcttcttgttgtataatatttttatcagaacgagacttagttacatattttggggtccaaaagtgaactttataactgtaaaattaaggtacatttagcagttccgtgctactttatctgtatctaatgactatccagtctgttacaactggaaccaaaacaaaatttctttcccacaactacctacgtgctgacaagtgagttctgttgcaaagtttgtactttgatagaagaaccctaagtgctctggtggtgtggatgccccatgctgtgcaatgatttctttctcccattattatcttatctgttcaaattattagggaacgtgctttcttacagtatagcacatgactgacatcctttgccctatatagacatgagcataaacacaaatgcaggcccagaaaatgcatggcaaaataagtacaatgaaccaacattttcttaatgaaatgaaaacaacactaacattttttcataggtaaattaaatcagagatcctcaggaggagatacccatggtcctctggaaaacactgatgggggagggttctgagtatttccaatcctgtttcttttcttttcttttctttccttttcttttttttttttgagacggagtctcgctgtgttgccaggctggagtgcagtggcgcgatcttggctcactgcaacctccgcctcccgggtgcaggcgattctcctgagtagctgggattacaggtgcacgccatgttggcgaggatggtctggatctcttgacctcatcatgatctgcccgcctcaccctcccaaagtgctgggattacaagcgtgagccattgcatctggcctgcagtcctgtttctaacagcattcgcacagacaggatggctccttttttggaatactataaaagaagtcttacttggtatcgaagatgcaattaagtcttgtctaagattttaagattctaattagacattaaaaccatgtttattataagtgtcctgaaaatgaagccactcagaaagctagttaaaccatgtttttgcccctttatctaaatctcgtattgtattttgtattttggcttcattggactaattgcgatttttctcttgcagtgagagggtccaagcctggtaagaatgtccagcttcaggagaatgaaatcagaggactgtgcttaaagtctcgtgaaatctttctcagtcagcctatcctactagaacttgaagcaccactcaaaatatgtggtaagtttggggcacacaggttatcttttgttacatgattgattaactgagtcaaggatatgttgatgaccttgtattagctagcagaatgattatcctgtttgcgaatagcttggaactacttaaaagtaagttaaatagtagctttagaataaatacctctaatggacttagtttacttagatgtacttcagatttttaaaaaaaatattttgctgaaggaataaacattgcaaaagaaaaaaatgaaactaagaatggagagctgaggaaaggtctgaactacatggatgtataggaatatgtttaagtttaaagcaacactggttatagctaaaaatagaacttccttgatactgattttgtggttctgttttctgcataatagaagcaaataatctgtgttttaactcaatgtaaaaccaaactttattcgtttgagtaatatattcttatagcatatttgcattgataaaactttgtgttcatgtttttaaaagtaaaatgttctttggcattatctagcagggagaaattgaaagctaaatgcattgatgggtggggggtgggggtggtgtgtatgtgtgtatgtgcttttgttcagatggggccaagactcactagaagtgcagcagttctgctgacacagtacccagagactgagctgtcagagatctgatattgatcagtcagcctgaaagggataccatgttacttggcaagaatgcatgcttggacataataaccctttccacagaataaattcttaatagttgaattttatgtgaggttctggacatacacatatacttaaaatttttgttaagctcagttgaactaaatatgatataagattggaaattacttagtgttggctgcccagttaaactctcattactagaggtcaatgccattgtacaatgaatatttaaagaatgtctttccaaagctttgttagtggattgagctttaagtatgggaacactgtgtaacaaaattcttgcttccaggtgacatccatggacaatactatgatttgctgcgactttttgagtacggtggtttcccaccagaaagcaactacctgtttcttggggactatgtggacaggggaaagcagtcattggagacgatctgcctcttactggcctacaaaataaaatatcctgagaatttttttcttctcagagggaaccatgaatgtgccagcatcaacagaatttatggattttatgatgaatgtaagtacttttctatatctaaggaagagttattgaaattaagatatttatcataaacaattatcacatacttctgttaggaattgtgagagtttagatgcagtggtatcatctctcatgttttctatttcatcaacttgtttttagcttagcttttttaggggggtgggggaacggagtctcactctgttgcccaggctggagtgcagtagcaccatcttacctcattgcaacctctgcctcccaggctcaagcaatcctcctgcctcagcctcccaggtagctaggactgcagacatgcatcaccacgcttggctaatttgtgtatttttttgaagagatggggtttcaccttgttgcccaagctgatctgaaactcctggctcaaggaatctgcccgcctaggccactcaaagtactgggattactggcgtgagccatgatgcccagccagttttttctctcttgggtagaaaaataacactgccgctgtaataaaataaggtcttggattcttttatgatgtgatgatcagattaggaagtaatgatcatggtaatggttgctttaagatttaaattgttgtattcacagataacttaattcttagtagaaatctgagaaccattccaaatagtttttttaagcaactttattatgtaatttcattccataaaattattctgttaaaagtgtacaattcagcgatttgtagtaaatttatagagttatgcgaccatcaccatagtccccaggcaaccactaatctactttttgtcttttctagagaatattgcatgcaaatggaatcttgcaatacgtaaataattagtttttgaaattattatagtttttctccttctggcatatccacacccaggaagaatggcaataactttcctaaaactaaaatgtgctgaacaaagattatttttacagtattttacacattttgtgacccattttggattgtttaattaaaggtatacttcgatttgactattgacaaaacacagttcttaaagctttgaaaataggctgggcacagtggctcacgcctgtaatcccagcactttgggaggccacagcgggtggatcacaaggtcaagagatcaagaccgtcctggccaacatgaggaaacctcatctctactaaaaatacaaaaattagccaggcgtggtggcagatgcctgtagtcccagctactcaggaggctcaggcaggagaatcacttgaacccaggagtcggaggttgcagtgagccaaaatcacaccacagtagtccagcctgggtggcagagtgagactccgtctcaaaaaaaaaaaaaaaaaaaagctttgaaaatatcagttcagtaaagggtttcaagtcagaaaaggttttgaatctttaccttcaccttccttttgtgctcatcattgctgccaggacgggtttcaggtttggagtgaagaggtggttaaggaacttcaggtacttggcatttgctaatatgattatagggatatcagcaaagggttgtccctgtggccctcccctagaccacatcgtaacctagagtctccagatgcagtgtttttccatctcaattcacatagatatgttgagctagcacatttgttagaagtgcatcctagaaagattatattcacaaactgagcccttctactaccaccctttcccacaactggatgtttgcttggtctgtgaccttgagatccagctgataagatggggtggcaggaatcatcagggacaaaaattctatgggttctggactgactgatagcattgaacttgtacatgaaaggtattcaatattaacagtgattctgtgtcagagaagtaccagtaagccagttactactccttaggtagtgaaatgcaaggcaggtgtgatgggttttttttgtttgtttttgagacagagccttaggctggagtgcagaggtgggatcatagcttactaccacctgtaatttctgggtttaagcaatcctttcacctcagcctccctaagtgctgggattacagtcatgagccaccacacccagccaacactgtgttttggatacttattccaagtgttactcttgagttttttgttttttgtttttttttttttttttgagacggagtctcgctctgtcgcccaggctggagtgcagggcgcgatctcgcctcactgcaagctccgcctcctgggttcacgccattctcctgcctcagcctcccgagtagctgggactacaggcgcccgccactacgcccggctaattttttgtatttttagtagaggcggggtttcactgtgttagccaggatggtctcgatctcctgacctcatgatccgcccgcctctgcttcccaaagtgctgggattacaggcgtgagccaccgtgcctggcctgctcttgagttaaaggattccttttacatagctgttagcccagcaaatacttttcatgtttgcactgatgaattcaaatccagttgacctcatttttgtgacgtgtatcctaatgtaatgatactggctgcatcagtttcggtttctacttaatgcagaggcctttaaaaaatgacttagaagatcagtgttggttggtatcagtttcgactcctgtttggtcatcaacacaatttgtagatcttatgctcaagcttcctctctctcatcaggcagatataatttgccatccagtctacttgtgggcatgaaagtgcccttgacataggtggcttttggttgaaggcttgcagacatttaatatatatataaaatacttaactaattttattaaaatgaaaagttgtattcaaatgttgattttcatgggacaatttaaaaaccactgcctgttttccaggtacataagtactttatactaccatttcttttttttttttttttaactttgtaaatgtgatggaaaggctgaggtatatgttgttgggtaaaacatgagtgtttgttgatctgtttctgctgccgtagctgtctgatgtacagtaatgcgaaaccgcattacaggttatggctcagaatcttaggatttggaagctaggcgattaccctggctaaacctttcaggagccagatgttcctagtggtctgaggtagccttagggaggaaatgaagagagaatagaaagaggcaggcccacgcttaaagtgctgagagggaaaaggaggaaacaaactgatttttgatggagaagccagcaggaatcagcaggagaagctggagtatgtggcataactaaagaatcaaagccggggcgtggtagctcacgcctgtaatcccagcactttgggaggccaaggtgggtggatcacttgaggtcaggagttcaacctgatcacaagaggtcaggaggtctgaccaacatggggaaaacccatctctattaaaaatacaaaaattagctgtggtggtgcacacctgtagtcgcggacactcaggagattgaggcaggaaaatcactggaacctgggagaccatggttgcagtgagccaagattgcgtcgcttcattccccctaggtgatgcagtgagaccccacctcaaaaaaagtatccagaagtgaagctgaccttggggaaagaggaagtcactgtcactggggagctaagacagcagctctgtttttataatacaggttaagtattccttaccgggaatgcttggcactagaagtgttgcaggttttttgtttttgtttttaattttggaatatttgtgttataattactggttaaacatccctaatccaaaaatctgaaatccagaatgctccagtgagcatttcctttgagcatcatgtgagtgctcaaaaagttccaaattttggagcatcttggattaaggatactcatcctgtggtggtagtagtagtagtattgctgttggaaatgggagttggtggtttttggagattgcccttcagcctttcgtaagaaggagtttgcttttttttgagacggagtttcactcttgttgcccatgctggagtgcaatggtgcgatcttggcttactgcaacctctgcctcccaggttcaagcaattttccttcctcagcctggcgggagaagtttgttaaactttccgaaccccctagtgaaggacctctaaagaaatatgcaacagggtgaaccatagtcagtgttagacccagtcattgagaaaccagaaattattaggagaaaagataacactcctctgcagagagaaggcaaacatttgttgaatttacatttcatttcaagccctgtggtaaactccctaagctgcttgatttgcattacagggtgggaggtgtggttcaggtcagttgcttgggctggctctaactctcaaaggagaggtataggtttaaacctttagatgtccctgtattccccaggaaaagagactaaggacaaactcttggtttgttttgtttgttttgttttgttttttgagacggagtttcgctcttgttgcccaagccggagtgcaatggcacaatcaaggctcaccgcaacctccgcctcccaggttcaagtgactcttctgccttagcctcccaagtagctgggattacaggcatgcgccaccacgcccagctaatttttatatttttagtagagacgggatttcttcatgttggtcaggctggtctcaaactcccaacctcagatggtctgcccgcctcggcctcccaaagtgctgggattacaggtgtgagccaccgcgcctggccaaactcctggtttttaaagattgtgcttagtatagtaagtacaggaaacaaaggcatctggacaattggacaatcctcaaccaatccagagaaatttgttcttgcaaggtatgtgactaggttttaaactcagaacatcagtttcctgtagaaacagtgcatagaggcatatttctgtcagaactgcactctgactccagaatgtgtggatgcctgtgctgttgagggaacatgaccatcctgtgacatcaggcctgtgggagcatgtggagtctgtgcagtgttggaaacaaatgccctgtagtgcctgtgagaggcagccactgccgtgtgatcattgtgcctcctgctagagtactgagggctggggatcctctggtgtcagtaggttattgaggggcatatatgggggatgggtgctaactctcaggtttcctcagaaggaaggtcataacgccacatgtgtaagttcgtaattgaattcagaagtaggggactagctgcggtgagattggacagagcagacaatttcaaacagaattgatgctcccagataattcctgggatttgatggcagtccttaatcattatcaaatctggaattctggtttcatgcttctgtggaatttaaaaagatttcatgtgaaaatgtaagtcattgcttttagatcagatatgtgtctctcctaaaccccttccctctaggagagtctttctttaaagagatggggtctcactatgttgcccaagatggtcttgaactcctgggctcaagccatcctcctacctcagcctcccgaagtgctgggattacaggtctgacccaccacgcccagctcttattttaaaagagccttaaaataagatggttttattaagattcttaataaaaacatagtttcatttgcttaaaaggcacccagaatcttgctttgggggaagttaatcctgagagaatgagcctacggaacagtaatactctctttttaatgtaatactctctttttaatcacaggtaaaagaagatacaacattaaactatggaaaactttcacagactgttttaactgtttaccgatagcagccatcgtggatgagaagatattctgctgtcatggaggtaggtgggctgatttttgttccaaataggttttgatcccttccaaagggtgaacactttaacatttgtgagttatcattgggatgactttctctgacagaacactctcctttgttgatgtgttgcattgcactttcactctgatacagccaggatcatttggataaaaatcttaaggaattgatttattacagggaattctagactctgtaagctaggtttgaaaatagagcagaaaatttttatttttattttattttttgtaaggatgagttctcaccacgttgcccagtctggtcacaaattcctgggctcaagcagtcctcctgcctcggacttccaaagtgctgggttacaggtgtaagccactgtgcccggcttagaaaagtttttggttttttttgtttttgtttttgttttttgagacagagtctcgctctctcacccaggctggagcgcagtggcaccatctcggctcactgcaagctctgcctcccaggttcacgccattctcctgcctcagcctcctgagtagctgggactgcagccacccgccaccacgcttggctaattttttgtatttttagtagagacggggcttcaccgtgttagccaggatggtctcaatctcctgacctcgtgatctgcccgcctcggcctcccaaagtgctgggattacagacttgagccaccacgcccggcagaagagtgtttttaactataaaatgaacaatgtgtaatttgttgtgccagagatcctctagaacactcaagatgtgagtagattttcccttgattgttttttatcctctcagaactttctctgacttggattttaaaagttattttggccaggcatagtggctcgcatctgtaattccagcactttggagaggccaagccaggaggattgcttgatgccatgaatatgagaacagcctataacatatcgagacccctgtcattagaaaaagttgtaaaagttagccaggtgtaatggtgcacacctgtagccgtagctatttgggaggctgaggcgggaggatcgcttgaacccaggggtttaaggctgcagtgagctatgatcgcaccactgcactccagcctaggcaacagagcaagaccctatctctgaaaatatatacatatatatgttataagctgttttgctttgctcagtggttgtgctttctataatgtaattaaaatagtaattgcaaagagaatttcttttcctgtatggtctgaaattaatcagccatcaaaaagcctgcataatgtcttttatctctgtgcttctgtacttaggttttggtagagttggatacatggtttttaactcttacaatatgcatcccatattgtaactcgatacaataagctcttgtagattttagccatcaaaaagcctgcataatgtcttttatctctgtgcttctgtacttaggttttggtagagttggatacatggtttttaactcttacaatatgcatcccatattgtaactcgatacaataagctcttgtagattttaatggtctgtgaacagtgcaggtttcctagagaactacctcacactgagcatccttatcttctcagctcagtcattataagccatcaatcataacatcgctcattattgtcatcaactgggggaagcccatagaagagttttttaaatcactggattttattttacagtgttctgtataatataattgtttccatgtactgtttaaacctactgcaccccatgcattactgcatgtaattctagtgtgtatccagttgtcctttttctgtgtttttttcttttaaccaaaagtaaataaaatataatcatagatttcagtttttatgtcaggattattgaatttctctgtagacaaatggaacttttgaaggggtagtatccttttaaaagttctgctttcttcattttttttttttcctcattgaataggtttatcaccagatcttcaatctatggagcagattcggcgaattatgcgaccaactgatgtaccagatcaaggtcttctttgtgatcttttgtggtctgaccccgataaagatgtcttaggctggggtgaaaatgacagaggagtgtccttcacatttggtgcagaagtggttgcaaaatttctccataagcatgatttggatcttatatgtagagcccatcaggtattgattttgaattttacatgtacacttaagagcatgtgtgtgagcacagattctccttgttgattttggtgggtaggtattgcttatttatacaaagtgtttctggaacacaagatatcaggctcaatgggaaactgaaacatggttttactcacctaatatttgcacctatttatattgaaaaaattttctcttcaaggtggttgaagatggatatgaattttttgcaaagaggcagttggtcactctgttttctgcgcccaattattgcggagagtttgacaatgcaggtgccatgatgagtgtggatgaaacactaatgtgttcttttcaggtagagcatgctttcaacatgatttccttttaatatgtttacactttgctgattgaaaaatataattgctactttttgcctagtgttcacttatgctgagtaggctaagtttttattttcccttaaccagtgtgtattatagttgaataatgtgtggatcccttgtaaaagaaaaaattaattatggcaccttttcccagtgttgaattataaatttttgcattgcatattggctccttaatgcttatggctcttttactgtaaatctaaaaccttaatgacaatgtaatgttcagctaaaattaaatcaccacttgaatgtgaaaagtactgagtgccttgtgctatgtgtttacttagttctgccctctagtctaacttccacaaaacttctgttaatgtaccatgacatcatttggccttcagttggcacaaaagcattatttagaatctgcctcagcctttttctcatttgcttattatgatacttgtacaacttcaagccagcctagtcataaacataaatacagccaccaaatcatgtggcactcatcagttaaatgaattaaaatagttttttaccaattatcagagagaacttgcagctttcaaaattccaaagattgtgcaaggatcccctacaggttggagaaacacttttgtaaagaactttttcccctgtatcagaaggagtagctaaagatactaccaggtgtagacttcagaagattgtcattatactcagaggcaaaataatttgtccagataatagattttatctgttcgtcatagcgttaattaccaaatgtttacattcaacatagcttgtgttaatatgctaggggggcttttacagttgttggggcagttgtgaacaccatctgaacagaagatctgaatgaaatttaaggactcttaagatttggggttgagtatatttcctaaattaactgtcttttgaatctccagattttaaagcctgcagagaaaaagaagccaaatgccacgagacctgtaacgcctccaaggggtatgatcacaaagcaagcaaagaaatagatgtcgttttgacactgcctagtcgggacttgtaacatagagtatataaccttcatttttaagactgtaatgtgtactggtcagcttgctcagatagatctgtgtttgtgggggcccttccttccatttttgatttagtgaatggcatttgctggttataacagcaaatgaaagactcttcactccaaaaagaaaagtgttttgttttttaattctctgttccttttgcaaacaattttaatgatggtgttaaagctgtacaccccaggacagtttatcctgtctgaggagtaagtgtacaattgatcttttttaattcagtacaacccataatcatgtaaatgctcattttctttaggacataaagagagccctagggtgctctgaatctgtacatgttcttgtcataaaatgcatactgttgatacaaaccactgtgaacattttttatttgagaattttgtttcaaagggattgctttttcctctcattgtcttgttatgtacaaactagtttttatagctatcaacattaggagtaactttcaaccttgccagcatcactggtatgatgtatatttaattaaagcacacttttccccgaccgtatacttaaaatgacaaagccattcttttaaatatttgtgactctttcctaaagccaaagtttctgttgaattatgttttgacacacccctaagtacaaggtggtatggttgtatacacatgctgccttcttggggattcaaaaacaggtttttgattttgaatagcaattagtgatatagtgctgtttaagctactaacgataaaaggtaataacattttatacaatttccatatagtctattcattaagtaatctttttacagttgcatcaggcctgaacccgtccattcagaaagcttcaaattatagaaacaatactgttctatacgagtgaccgattatgctttctttggcctacattctttattctgcggtgaagttgaggcttataagttaaaacaaaggaactaacttactgtccaccagtttatacagaactcacagtacctatgacttttttaaactaagatctgttaaaaaagaaatctgtttcaacagatgaccgtgtacaataccgtgtggtgaaaatgaattcagacttattaaatgatgaacttgttaaatcttctcagtgtctatttatcagcacaatacacacaggagaactgttgatggcatattgaatagattttcctgaataaattgctctggaaaccacacggttgcattttggttttgcttctttcaaatagtctgttttccaaagatggattataatacagatactaatactctttgagttgtaacctggagacatctatttgtgtaatagagtgatgaggtggaacgatcctcctctaccatttggggagatggggattaatctgtgtccatagttttcttactgtctgggttcacttgaacttagtggcgaatttgaaagcagctcactttgttttctgagtcagccacctatattttcatgcacagttctttgatctcttcccccttccctggtttatatggaacacagtatcatgtcatattttgtcacaagtatgtatcttaatttctcaaaggtacgctaaaaatgagtgaagagcatgtcatttgaaaagctttttttcctgttacaaagtggtctccctgagggcgttggcagaggacctagcgaggaactctttgaaggacacagaagagcacagggccatggactcccctctgctaacaagccccggtcctgaggcatctggaccctgccatgctcccatcaaagagggaagggggacctcattacctgcctgctccacattgacaatatccaaacttgtgctgtttcacttttacgtaatattctcccagacttctttgactataggaagaaaacagtgaatgataattgtgtcaaaggcaaattctgcacatctttatgtcctgaatgttaatgaattgatactaggatttcaaaatgctataaagtaaaattgggctgctctactaggattatatcattattggagtgtggtatcaggctcaagtttttcttaaggaacatttatcaagtactaaggattatatcattattggagtgtggtatctggctcaaatttttcttaaggaacattggtcaagtagaagacaagattaatctttggttttaggacctgagatcattaggagagactcagtttgaaatctgtttgacagcagaacttgagatgtgtgtgataacccaagtcttggcccctcctacctgatttgagtgagtgacctgaatgctgagccactgtgttcctagctatgaaacgtccatagcagaagccgtctcacattgtgaatcttgagtgaagttgtgtggtgtctttagaatgctcaactcggtggtatttgctaaatgtttgcttttggttatcttatcgatcccagttctttcgtgtctttgcacatctcggttggctgagtttgaagtgtcacttgcctttgctttgctttgcacctgataccaagccatggatagctaagtatgtctgagcatttactgcataagggaacaccatcatggatgttttcagcatatgcaaaagaagtaactaggaaagaggaagttgtaattattagtgtgatactattctattaagtgttttggtttggttttgtttttgtcaagtattctgggtcacaagatgacagcaccattacaaaaccatacagaaaggcaggtgctaggcatccttagtcgctcaatcatctatgtaataccctcccatagaaattgctgatttggtacacaaactaggcctgcccatgtctagttgcagtggacagcacagatgctcacatataacccttgttcttgaagtaccttgatttaaggccagagttctcgagtaacatacacataaatgggatatggaacacagggactaggaaaaaattactgaatgctgagccatgaagaaggcacatgatttaaactgatggaaaggaatatcaatcaaatttctcaatgtgggtccatgatgagcgtttgaaagggaattcagtttgctgggaagtgaagatacaagctaccaagttaaggcatcatgataccaaaaaacacaaaggaaccattgcagggaagtgctgcatgaaaaatcttaggtttattttggcagtttgcagacaccatgtgaattgagacacggagacaagggccatgtaatccaagtatcacctgaaattagtagaagggacaaagctgaggatctgcatcagaacaaagaaggaatgtcttcctcatgtttgggtctatagaagacattaaagaaaacttccagaaagtgggtttgaggccgggcatggtggctcacacctgtaatcccagcactttgggaggctgaggcaggcggattacaaggtcaggagatcgagaccatcctggctaacacggtgaaaccctgtctctactaaaaaaaatagaaaaaattagccgggcatggtgatgggcgcatgtagtcccagttactcaggagcctgaggcaggagaatggcgtgaacctgggaggtggagcttgcagtgagccgagatcacgccactgcactccagcctgggcgacagagcgagactcttaaacaaacaaagtgggtttgaagtgctatgtctccattttcctatcttgaggaaaagacccaaaactcaacctcatgtaccagctggcttgaatctgcaggggtggttttgatgagtaagaactttcagtcctgcaaatcagatgtatgcatcagcccagactggaagaacatggcaccctctgccccaggctttggtttccttctcacccacagaagaggaataactagaatacttaacgatgggattgtgaggattagcccaccaaaaatctacaacttgccttagtcataagcattcaggaaacagccctggtataagaccaatgaatagccctaagatttagaccaatggcaacacacaggaactttgattaggaaaacttgggccaggcacaggcagaagtcacttttcattcatccttgccaacgtgtgagcttcagaagagtggaaatgatggactatcaacaccttcagcctacgactcaaatagaattgagtacttgcattagtgcatccctgaattagtgggtggttcatagttgtgtgatcacagagatgactgccatcccatctgatcagctgtagctccaacggacttgttgtcatccagatgtattacaagtattccaataatttcaaacacaggtaagtatttcagttgtgagaattatgtcagttatttgaaagataaatatgttagaggattaaagcacttcagttaatcatgtaaacagttttgtaatcaaaaagttaaaactaggctgggtgcagtggctcatgcctgtaattccagcgtgattgggaggccgaggtgagtggatcacttgaggtcacgagtatgagaccagcctggccaacatggtgaaatcctgtctccattgaaaatacaaaaatcagccaggtgtggtggcacatgcctgtaatcccagctactcgggaggctgaggcaggagaatcgcttgaacctgggaagcagaggttgcagtgagccaagactctgccactgcactccagactgggcaacagagcgagactctatctcaaaaaaaaaaaaaagttaaaactaagaaagtttgcagtaatagggaaatagaattttacctacataagttcaagaactttatactcttgaactaacaaaagtttgtaagaattttaaaatataaggcatgcaaaatatcaggaatgaatcagtacggcaagtcttttaagtgcatctcagaaagcatcctgaggcactgcccaaagcttgagattgaggggttgtgtaaaggcactggcatcaaccagattttttaagtctaggaattgagaaggaagacataagtctttaccttgctattgggatggaacaaccaatttggtggtaggaccagccaaaaagcaacttgcctttccaccaacctcaaagtgagcatctatttagtattacaaacatgcagataccgcaggagcaataaagcgacaggtggtgctaagaaggtagcatactggccgggcacggtggctcacgcctgtaatcccagcactttgggaggccaaggcaggcagatcacctgaggtcaggagttcaagaccagactgggcaacatggagaaatcccatctctaataaaaatacaaaattagcctggcatggtggtgacacatgcctgtaatcccagctactcaggaggctgaggcaggagaatcgcttgaacccaggaggcagaggttgcagtgagctgagatcgcaccattgcactacagcctgggcaacgagcaaaactccgtctcaaaaaagaaaaaaagtagcataccagcccagcatcaaacttgggagaattctgatgattgtaataattaaatggtatcaattaaatttttttccagtatgggcaacatggtgacaccccgtctctattaaaaatataataatacaaaaaattagccaggcatggtggtgcacatttgtggtcccagctacttgggaggctaaggtgggaggatcgcttgagcctgggaggcggaggttgcaatgacctgagatcgcgccactgcactccagcctgggcgacagagtgagaccctgtctcaaaaaataatttttgatagaatttcaaattcattcttgtgttccaaacaatcagtttcctaatagcgatatttagctatgaccaccaaatactgattttaatactcattcagccaaaatgcatatttggtgcaactcttgaaacaattctaaaatgtcacttaaaagtgcgtattccttttttttttctagccacccaattagcacagtatcaaacaccatgtctcccagagaggaccatcccacacaccagttgttcagggctggtgaacaagagaacagtaaactggtcgttatattcttggacagactggcttgacctttccctggcaagttaggtaggtgtggcagagggagcgggtgggataaagggtgtttttgctgaggttggcagaagatttttaccatctgatggggctttgctcattcaccagctccacaagcccctcaaggagtcctgttccatacctgctgttagtttaccataatgaaatcacttcccaattttttttttttttttttttttttttttttgagacagagtcttgctctgtcgcccaggctggagtgcagtagtaccatctcggctcattgcaagctccacctcccgggctcaagtgattctcctgcctcagcctcccaagtagctgggattacaggtgcgtgccacacacccagctaatttttatatttttagcagagacggggttttactacgttggccaggctggtctcaaactcctgacctcaagtgattcgcctgcctcagcctcccaaagtgctgggattacaggccttcccaagttttgattactgacctacaaacaccacatatcttctctagtgcttgcccttcagattgagaggtggaatggacagatcgtagactcacatggtcctccgctcaggaggctgtccagcataagtgaaggacccagattcagcgtggatcacagggttagctggattttactcactagcttggcttagattggtttcagatctgccccatgttcctctcactgttctagggccattgggtattacttaggagggtggactgaggagaggagagcaagcaggcaatcttaagatttgctgcagtgcaaattactcattgggtgcacattttggttgccatgaatgctgtgtttttatacttttatttttagaagccataatcttgctgaggtcaatataatcttgctagaatgaaagtttttttgttttcgtttgtttttgagttgtagtcttgctctggcacccaggctggagtgcagtggcacaatcttggctcactgcaacctccacctcccaagttcaaacgattctcccacctcagcctccagagtagctggaactacaggtgcccgccaccacgccgagctaatttttgtacttttagtagagacaggatttcaccatgttggccaggctggtctcaaacccctggacctcaggtgatgcgcctgcattggcctcccaaagtgtgagccactgcgcctggcacaagaatgaaagttttgagaaagtgatctgcattctccactccagaaaaccagaattcctcttaagacaatgctggtcgaacttttttgacaggtggaacccatcaaacaaaataatattggaggcctcatgtaaaatgaataaaatcagaacagctctgttaaaagcaggggctgggggctgcttgtccccgaacagcttccacctccaaagattgaaaccacgctctaggaaaaatattggctgctacatacaggcctgtgtttgtagaccagcattagtaagatgttagtacaacttattaggtagagccttccctgattttgtgatgccttgtctaagatccaaatagctaagaagttcaatccaagtattctccaagtagaaagtcaacacatggccaggcacggtggctcacgcctgtaatcccaacactttgtgaggccgaggtgggcagattgcctgagctcgagagttcaaaaccagcctgagcaacacggtgaaactcatctctactaaaatgcaaaataattagccgggcgtggcagcgtgtgcctgcagtccaagctactcaggaggctgaggcaggagaattgcttgaacccaggaggtggtggttgcagtgagccaagatcatgccactgctttccagcctggacaacagagtgacactacatctccaaaaaaaaaagaaagtcaacatgtaactaccattttccaagttatttgctggttttcatttcttttttctttttttgagatggagtttcgttcttgttgcccaggctggaatgcaatggtgcaatctcggctcaccgcaacctccacctcccaagttcaagcaattctcctgcttcagcctcccaagtagctgggattataggcatatgccaccacgcctggctaattttgtatttttgtagtagagatggggtttctccatgttggccaggctggtctcgaactccctacctcaggtgttccacccgccttgccctcacaaagtgctgggattacaggtgtgagccaccacgcccagcctggttttcatttcttagcactgtggtaacagacacaggagatcagaaaagcattttgcagatttgaacaaagcatcctttaggagaccagggtttttcatcaaggggatttcagtgccccccctttttttttttttttttttttttttttttttgagacagtttctctgtcgcccaggctggagtgcactagtgctaacttggctcagtgcaacctccgcttcccggattcaagtgattctcctgcctcagcctcccgagtacctggaattacaggcacccaccaccacacgtggctaatttttgtatttttagttgagacgggttttcaccacatttgccaggctggtctcgaactcctgacctcaagccatccacccaccttggcctcccgaagtgctgagatgacatgcatgaaccaccacgcccggcctgtgtgtctgtttttcaatgtgtgtgtatctataaggtatattgtcattttatatgttattttatataatatatatggtcccgtgtcacttaacgatgggaatacattgtgagaagtatgtcattaggcaatttcatcattgtgcaaatcaccatagtgtccccacacaaacctagatgacataagctatacggtagagcctattgctcccaggctacaaacgtgcactggaccctgtaggcaattataacacaatggtaagtatttctgtatctaaacatatataaatatatagaaaagctacagtaaaaatatggtattatagtcttatgggaccaccatcacatatgcggtccatcattgactgaaaactcgttaggtggcatgtgactgtatttttaatggtttgttgttgttttgttgttgttgtttgagacagagtttcattcttgttgcccaggctggagtgcaatggtgcgatctcggctcactgcaacctccgtcccgcaagttcaagcaattctcctgcctcggcctcccaagtagctgggaccacatgcatgcaccaccacgcccagctaactttgtatttttagtacagatggggttttttccatgttggtcaggctggtctcgaactcccaacctcaggtgatcctcctgcctcggcctcccaaagtgttgggattacaggcatgagccactgcccctggctagtgtattttttaaatgaaatgtcttaatacaaaagaatacataaaacacccacatacccatcgcctgggttaagaagtagaatattaccaataacttaagagcccgtgtgcgtacctccccagttacatacttacccatctccctcgagagttaaccatgatcttgaatttgaaagtaatcacttccttacttttcttcattgtgtttgtatccctaagcaatatactgttaaattttgccaatttatttatttttatagaaacagggtcatgctatattgcttaggctggtctggaactcctggcctcaagagatcttcctgcctcgggctcccaagtagctgggattataggtgcaaaccacagtaccaggcaatttttgagcttcatagaaagagaaatatatgtatttctgtaaacagtttccttcagcatttaaaaatttttatttatttatttattttattttgagacagagtctcactctgtcgcccaggctggaatgcagtggcaccatcactcactgcaacctccgcctcctgagttcaagcaattctcctgcctcagcctcctgagcagctgggattacaggcacatgccaccatgcctggctaatttttgtatttttagtagagacggggtttcaccatgttgtccaggctggtctcgaactcctggcttcaagtgatccacctgcctcagcctccttttaaagtgctgggattacagacgtcagccatcctgcccggcctaaacatttttatttatattttagagagacagggtcttgccttgttgccgaggctggtcttgaatttctgagctcaagtgatccgcccaccttagcctcccaaagtgctaggattacaggcatgagccacctcacccagcaagcattttttttttttttttttttttttttttgagacagagtctcattctgtcacccaggctggagtgcagtggtgcaatctccgctcactgcaagctctgccccctgggttcatgccattctcctgcctcagcctcctgagtagctgggactacaggcacccaccaccacacccagctaattttttgtatttttagtagagatggggtttcaccatgttagccaggatggtctcaatcttctgaccttgtgatccacccgccttggcctcccaaagtgctgggattacaggtgtgagccaccgcacccagcctaatttttgtttttcaaaacaaaaatatttttgttttgaaaatttttgaaatgtttaatagcgacagggtttcaccatgttggccaggctgggctcaaactcctgacctcaggtgatccgcccaccttggcctcccaaagtgctgggattacaggcctgagccactgcacctggccttaatttgcattattaatattagaggttatctttgccctttttcctattctctttgggaaatataatgaggtgtattagacctcattctgtctttcatttcactttaacatcttgtttttttccatctctttccctatggtttgcattcttgataatgtcctcagctctatcttctagtttactaattttttcttcagttgtgcttaatctaatccatgcactgaatttttaattttaattactacatttttatctcaagttctattgggttttttgttttcctttattattattattattattattattattattattattattattttggagttttttgtttttcacaatccacttggtcattcttcattatctattatttcttgctaatctttttaaaacttttaaaatttgaccaggcgcagtagctcatgcctgtaatcccagcactttgggaggctgagtcgggtggatcacttgaggtcaggagttcaagaccagcctagccaacatagtgaaaccccatctctactaaaaatacaaaaattagccaggcacctgtaatcccagctactggggaggctgaggcaggagaatcacttgaacgagggaggcaaaggttgcagtgagccgagatcacaccactgcattccagcctgggcaacagagtgagactccgtctcaaaaaaaaaaaattatttgaccccatggccttctggtctgacgttttttggccgggcaccatggctcatgcctgtaatctcaacactttgggagctcgaggtgggtggattatgagatcaggagttcaagacaaacctggccaatatagtgaaactccatctctactaaaaatacaaaaaattagtcaggcatggtggcacatgcctgtaatcccagctactcgggaggctgaggcagagaactgcttgaacccgggaggtggaggttgtagtcagccgggatcgcgatactgcactccagcctgggcaacagagggagactctgtctcaaaaaaaaaaaagttttatttctctaaatataataaacatggttattttatgttctatctgattattccaatatctgaagtgtttgtgggtttgtatctatcatttgtgtgtttttgtggggggttttgccattcttctgaatgtttctatcatctgttgtttctattgttcctcatagttgtttctctgtgtgtgttgtgattttttgacagagttgcccattttcttcacatctttatccatgggattctctgcggcctgaattgaagttgagctcctccagagaagatcagagtttgcctctgctggtagactggggcagtatcaacctagactttaaatcctcacttgctcacttgacatttttcttttcttttcttttttttttttttttttgagatggactctcgctctgtcacccaagctggagtgcagtggtgcgatctcagctcactgactgcaacctccaccccagattcaagcaattttcctgtctcagcctcccaagtagcaggatttacaggtgtctgccaccatgcccggctaatttttgtacttttagtaaaggcggggtttcaccatgttggccaagctgttctcgagctcctgacctcaagtaatctgcctaccttggcctcccaaagtgctgggattacaggtgtaagccaccatgcccagacccacttgacatttttcagatcatacaagtctcatgaaattaggccacaaacctgcagatcctggttgtgaattctcaggagaaaattgttttgccaagagtaatggagatcccattggtaataactgtaatattttcattgtgcatgtgtttgttttgatgaataaacattggagtcatatatatctttcagccatga

SEQ ID NO: 10 - DNA, 3246 bp, Mus musculus
aggagagggcccggagctggtgggccggagcggcggcgccgccatgtccgacagcgagaagctcaacctggactccatcatcgggcgcctgctggaaggtgggcaggggtagggaaggtgcgctcgcgccgagcccgggcccctcgccgtctggaagtggcgcgcgggcggcccgccgctcccgggggctagcgtcgcttgggcggggacaggccgtgaggatccatggccacggcaccccccccccccgcgggcgcctgggctttggagcgcgctcagaggccgggggtgcacgagtgcccgggactcagggtgaggctcctgcgaagctagctgggaagggatcccgaagcgaggaagcttcggctgacaggctgctgagcacaggcgtttctctggaggccctccgggtggctaaggggtcggatcttgcctttgaaggcctgacgcaaaggtttcggggtgagggggtgggggctcacttgcaacagttgcccctgttaggggggcgcgacacctaccctcgtccggtctgcgaggtcagggtcccaagaccctattccgtactcagtgcagggctcacggcctgggaagaacgtgcagctgacagagaacgagatccgtggtctgtgcctcaaatcccgggagattttcctgagccagcccattcttctggagcttgaggcgcccctcaagatctgtggtaaatccttgcacagaacccttttgcttcctgaaaatagcccctactcgtgccccattaaccgtgttcctccaccctttaacctattgctggccctggggtcaagtttacatcttgcacctacctagttttctctggttcccaggtgacatccatggccagtactatgaccttctacggctgtttgagtatggtggcttccctccagagagcaactacctcttcttgggggattatgtagatcggggcaagcagtctttggagaccatctgcctgttgctggcctataagatcagatacccggagaatttctttctacttcgtgggaaccatgagtgtgccagcatcaaccgcatttatggcttctatgatgaatgtaagtgctttgaatgttgctgtctgcccagaactacattccctgctcttcatgggccccctacattggtctagtgccaaaagacccccaatgatgagccctgtagggccccagcaacacgtctgtcgtccttggcgctcaggaggggcctggtgaatggatgtacatgcagggctgcccatttgctaaagccacagccctgcctgatgacacccccttctagtacattccttagtaaacatttaggatgcctgtatgaataaaacagacccactcctggtttggcctcatccatgggaccagagtgggtaccaccatcatggttgggccagacgagttcaggtgttggaattctggaaggtagatgagggtagagcaaagagaagaatgtaagctttctttgcttttcggtctttttttctcagctttgtccctggatgggtgcacagaaacctagactggggttgggggttgggggtgtagtcttccctatgagccgcttccttttgcgcaggcaagagaagatacaacatcaaactgtggaagacgttcactgactgcttcaactgcctgcccattgcagccattgtggatgagaagatcttctgctgccacgggggtgagagtggttgtgggtctgtgcttgggttcttgggccacatccttaggggccttgtaaagttaacagaactcttctcagttccctggaatagcgctccttgaatctcatcttggatatcgcttattaggggagattaccacacccccgtttgtttttgagactggctttctgggtaaaaccctgacagtcctggaactcactttgtagacctggttggccttgaactcactagagctcctgcctatctctgactcccaagtgatgggattaaaggcatgctccacccaccagtttggttgcactctttcctgttgtggcatgaggcatcctgtagtcaactcattaatctccttctgcccccccacccccgaggtctgctatctgtacttctaagtggtggtgtcatataaagtgagttccaggacagccaaggccacagagaaatggggccagtggggaagtaatggattctgtctccctgaacttaaggagcccagattagcggtgggaacagtggagctgtcccaccccagagctgggcacaggacagcacttggtttaaggaacactattcccactccaccacccccaggcctgtctccagacttgcaatccatggagcagattaggcgtattatgcggcccacagacgtgcctgaccagggcctactgtgtgatctcctgtggtctgaccctgacaaggatgttcaaggctggggcgagaatgaccgtggtgtctcctttacctttggggctgaggtggtagccaagttcctgcacaagcatgatttggacctcatctgcagagcacatcaggtgggctggtggcctggggcgggctgggggtgggggtggggtgcctaacttacagactaaaccgttcctgccctggcccaggttgtagaagatggctatgagttctttgccaagagacagttggtgacactcttctcagctcccaactactgtggagagtttgacaatgctggtgccatgatgagtgtggatgagaccctcatgtgttccttccaggtgggtgtggggaagaggctgcccaatagacgcctggctcaaccccaggaccctgatgagtgttttctcttccagatcctcaagcccgctgataagaataagggcaagtatgggcagttcagcggcctgaaccccggaggccggcccatcactccaccccgcaattctgccaaagccaagaaatagcctccatgtgctgcccttctgccccagatcgtttgtacagaaatcatgctgccatgggtcacactggcctctcaggcccacccgtcacggggaacacacagcgttaagtgtctttcctttattttttaaagaatcaatagcagcatctaatctcccagggctccctcccaccagcacctgtggtggctgcaagtggaatcctggggccaaggctgcagctcagggcaatggcagaccagattgtgggtctccagccttgcatggctggcagccagatcctggggcaacccatctggtctcttgaataaaggtcaaagctggattctc

SEQ ID NO: 11 - DNA, 34866 bp, Mus musculus
ctgttcgttgcagtggaacgcctgagagagggaatcgctcctgcgcgttagcaaccgcggaggggagggcggggagctggagaggcaggaaggggacgtggcgcgcgtgcgcgcagttcggcgcggctaggcggcccgcaagggcggaggggagggagtgacgctgagggcggggctgtccgtggcgctgccgagctttgcggagctgggcggtgccgaggaggaggtggaggaggcggtggcggctggggtctgacgcggcccggttcctgacgcggcccggttcctgggggcctgcttgtttatttatttatttccagtgggcgccgccagcgtgtgcgcgcgctgtcgctgctcggcggggagggggtggggggagggcccgcgcccggggggagttggagccggggtcgaaacgccgcgtgactcgtaggtgagaacgccgagccgccgccgccgccgagaagccctgttaacgctttagggaggagagtctggtgccgacaagatggcggacggggagctgaacgtggacagcctcatcacccgcctgctggagggtgagtgcggcgccggctcgtctccccacccggccgcggggcccttcccgctcgagctgaggtcggagaggagggcaggcgggcgggcgggcgggcgggccgggtgccccaggaggccaggagcgcgggctggcgggcgggcaggcggggaggccgggccgagcgaggccggaggcccatcggctggtacagtcttcccgagaggcggccgggctccgcttggccgctgtgctggcctctgcagaccgttccgaagaaggggcgaggatgcctgccgctgggctttgtgtgtccgtggaatgtgtatttctcggttctctttgtgcgttgcccttggcagcctccgatggtcgttccggtagtgaattttacataaaaccttcccagtaaggctctcggatcgtttgcattttactggatgctctagaaagaattagggagaggatacgatgactagtttaccatttattatctggagtataaaaaaacagtaaaaatcctgaatcaggttaaagacgtccgttagaaatacgaaacgcccggatacatgtgctgaaggggttgatttgggggcttttttttttttttaaacatgtttgagccgagtttaatttctgcacaaacttagcaaatgtctgttgtgaataatctcttgagcttaaaaggtattttacaagtgttctcagcatttgaagttccgggttcttatgcttttatcgattagttttgtgttggtgggaacataaggagaaaagaggagatggagaggccagtagttaaatgtattggtccgttgggggatactgttcgaagttaaaggcgggtcttggtcaaaagaacccttttacgctataaaatttgcgtcgtcactaattgtggagtgtgtaaaaaagaattgcgtttttgagtggttaagagtcctttttgtcagcctcctaatgaatgcgtgtgtttactacagcgaagctgtttgtgtttggaattttaccaaagagcgtggagaggggtgacttttctttaactagttaaagtacgaagtatttattctctgtttaaaacaacaacaacaacaaaacaactctgtttatatgctccagatgtaaaagctttccagcagccttaaggctgagaaaaaggcagaacttttaccaggaactctttcttatttaccttctcctttagtatcaagttattcctgggaggccccctggtgcttgctgtgtttactgcttgggattttgtggtctttaattgatagaaacatcttgctgggtctgtgagaaatttgtttagttaaaaaaaatttatgcttgtagtacatggctggctctattagggaaatatagttattttaaatctttatatcttgttactcttcggagaaggggaatttaagcccaccactgttttcatataatctaaattttaaattaaatttggatcagattttatcgcagtttattaatatttttaatttttcataaataccttatagcagatgcatgtattttaaaccgatctagaagcctttaaatcaaatgtacttctatacagttacagatgcttaaatacaactaatagagtgggagttgtagagaaaaaggggaaagtagcctgagctatatcattgttaaatatttggttctatcatcagtcaccagcacccaccagtccccccttcttgggtaagaatacgttctttttttctaatatagcaagtgtttattgactgcactgccaggcaccaggagtcaaagataaggctcactcctcactcaagcagctcagtataaacaagtggcttctcagtgtgtgctagccttcctgctcttcctgctttgaatgtacaatttattagtagttttgttacattcttgttccttaatccagacgaagactgcttttgttgttactctaaaatattaccagtccctccttccctccccaagcctaaacctgttcaaataaattttgaagtcttggattttctagtgagtaaattcaatcttgaaaaaaaaattttttttgagacagggtttctctgtacctggctatcctggaacttgctctgtagaccaggctgcctcaaactctgcctccccaaatactgggaattagagacatgcaccacctctgccgggcagcctactttagttttcagtattacatgctaatttaactcaaattcaagtggtgctattctgtaaagttgcctcgcagaacagaataacctggttatatattttgtgcgtcctaaatgaaagtgaacattaaaagatacataattatagttaaagatgtctaggttatatattttgtgtgtcctaaatgaaagtgaacattaaaagatatgtaattatagttaaagacgtctatccatagcaagactacagttagtggtcattgtattgaaagtgtgaagagaaccttcgagaatgtgtgctctaaggaccagtatgataaatatgacatccttggtagataaaatgtgtggattcagtgtgttcagccatttcacagtgtatgcacttaaaatatgtgttcctaaaagatgcagtgttgtctatcaattaaagattgcttttggctgggcaaaaagttgtggctttaatcccagcacccagtaggctgaggcaggcagatctctgagctctagaccagcaaaacacaacaggaaaaaaaccttgcttttgtaaaaatgtttttttgcattaagagaaaaaaattgatcagaacctagtctcattttaaagtgtttactttatgttacagtttattcatttaaaaatatattaaactctgatggccagttaagtgcactcactttcagtaactactttttatgttactttgttcgatgttagaccatttaaataaaaagactagaacattgtttggactgggtgtgcttctccagtattattttcttggtttaagttttacttacaactaaacaataactgttttgccatctgtagtgaccctactatagtcttgtgatttatcataattgattacataagttcagattagttctgtgagtgagctagaaagcctgcctagtaaaaggttttgcttagagtggttgttctctagatatttgaagaggaagtgggagtcacctgggggccttaggtaacattccaaatgagtttattttgaaagaagtcaaaagtaagtagaatcctgatactttgtgaaagagaatcaaatggtgcccttggaggccagaagatgccctagaactggaattacgatgtgatgagccgccctgtgggtgctgagatttgaacccaggtcttctgcaagaacagccaggctcttaacggctgagccattgctccagcccaaaccttcccttttaaagacaactttgagatgcagttcatatcccacatggtttgcctattgctgctgtgttagtcagtgagttttagtgtgtccacagtgttttgcagctatcaccactgtcagctttagaacattctccctcccttccacctccccccacccccacaagccctgtttcttttttcttcatctgtctccctctaggcagtagagcggatcggatcgttaatctgccttctttctccagatttgccggtttatgcacatattatgtaaatgaaagtgtatataatgtggtcttttgtgactaattttcttttgagttttttgagttcaagtttttgagacaaggtgttactgtgttgcctttgctgtccccaaactcagagatcctcctgggattaaaggctctgtaactcctgggcctcttgtaatgttttcaagtttcaactatgttataaaatgtattagtacttcattccttttatggctgaataatactccattgtgtctgtgtaacaattttcagaaatactcactggttgatggctatttgagttgttttcccttttgacctttgaggacgatcctgctctcaacttgtttttgagtggatttaggctttcagttccctaggctagattgctgggaggggaattgttacatggtaactcggtgtttagacttttggaaaactggagagagttaaagcttctttattgattacattgcttcaagttcaagcccagtagtttaaaagacaggttgaagcaggtgccagctgctctggctgcctcttccttcacagggaacatcttcatactgtgtgtgaacatgaggtaactcattagaagtaagagcaggctggagagtagatcattggaggtagactttcttctcttgagtgttctgggtgagtcatagatagaccaatccctgtttgtccagaaagtaaaggaaactgatttgatttgagaaaaaaaccaaaaaaccaaaccaaaacaagaaaaacctccagagatattttctgtgacatttccaaataattcattgaaatttagagacctattcgaattactatctgcggagagaagagttactcccacccctcctttaattactaaaggacattatgttgaagatactctaccggtgagatacttctccagctcttttatttttatttatttatttatttatttatttatttaatttttaacaagttttgctaagagagtagccattctggccctgagcttgccctgtatcccaagcagaccttgaactttgaattttctgacctggactcccaagtagctaggattacaggtgtatgccctcatactctttacatttatcatttgactattccattttcagtggccatggtggcaaattcataccagtaaatagaaacgttaaggtatttttatatgatgaaactcccattgagtatttctgaaacaaaagttttagaattaattcagtgtccgtaggcaagaaatggtttactgaagtatgtttggtagactcttgactatcctgggttttttagctttgttagggtgcaaatttctaacctttggcaaccacttttttttgtaactataaataaccatttcagggcttgtttcttttagaaaaaaaataggatccatgaaggttggaaagagaggtcaaggtcactaatctttctgtgtatcacttcattctgaaactgagttgccatgtttccaaagttgtttttagtagatgaagtctaatttttacagctgttttctgaagtacattccaaaatagaatctccatttgattaatgagatgggaaaactgagatagtctttattctattatgggaagtaaaagacccaaatggagaaacctattctgaagggaaagaagttataacagcactagctaggtttggtgggggatgatctttaattcttggtaggaatttgtaatgcacgggtgttgtcctttcttctagcttttcatgtgtgtagcacagtgatttataaagtagtcctaacttccctgagtcactggccagagagattttcaaatttccttgccctgtataataaggaaggacaagaagacgtgtgcacagcgggaacatgcttttggtggttggtccgtgttaagattgtaacagggaagttttgacccagcttatttcaccataaaggagttttattccaagagcattaaaatatcccagtgtaacagaagctaccaccagaatacctacaaaacataagaacttttatgattaatttatttgatacaatagtcattgcaatttagtatgtgattatttgtgaaaaaaataacttcataaattattgaggatgttgttattcatttgtgtaaacctaaagtctgcacaagagacagctaaattcttacctctctttttttcattgttgttggttattgtattttgtttgcttgttttgagacggggtctaatgtagcctttgatggtttggaattttctgtgtagataaagctggtctggaacttgcagggatttgcctgcctctgcctttgcctcctgggtgtcaggttcagtctgttttgacccatggccttggctgaaatatatgaagaaaatctgtatttgtattgtatctatgttggtattttaaaattacatttattttgtatgcatgcatgcatgtttgtaaaagtcagagggcagcatttggaagctggttctttgcttctaccgtgttggatctggggtgccaactcagctaatcaggcttggaaacaagcagctttgcctgtatctgtctttttactgaattagaaaatggtgtctttgccagccatggtgcacatatgtaatgtccaacactggaaaggcagaggcaggcacatctctgtgagttaaagttcaccctgatctccacagtgaattccaggtcagccagggctcatagtgagataccatctcaagtaagtaagtaagtaaatagccaactgttttagacaattgtgagtactttctcgtactatcaagaatcaacaaggggctttttcaattgcaatacaaattataatttttttctacattaacctaaatctatcttgtactgtaaatgttctacacatgacttggtgatgggcattggtcgtttggaaaatggctcactgaattatatagatttaaactcatataaatgtcagtagagctcatcagaagagacttttttccatttttattaggtatttagctcatttacatttccaatgctataccaaaagtcccccatacccacccacccccactcccctactcacccactccccctatttggccctggcgttcccctgtactggggcatataaagtttgcgtgtccaatggtcctctctttccagtgatggccgactaggccatcttttgatacatatgcagctagagtcaagagctccggggtactggttagttcataatgttgttccacctatagggttgcagatccctttagctccttgggtactttctctagctcctccattgggagccctgtgatccatccattagctgactgtgagcatccacttctgtgtttgctaggccccggcgtagtctcacaagagacagctacatctgggtcctttcgataaaatcttgctagtatatgcaatggtgtcagcgtttggatgctgattatggggtggatccctggatatggcagtctctacatggtccatcctttcatctcagctccaaactttgtctttgtcactccttccatgggtgttttgttcccaattctaaggaggggcatagtgtccacactttagtcttcattcttcttgagtttcatgtgttggacatagtactaccggaggatccagcaatacctctcctgggcatatatccagaagatgccccaactggtaagaaggacacatgctccactatgttcatagcagccttatttataatagccagaagctggaaagaacccagatgcccctcaacagaggaatggatacagaaaatgtggaacatctacacaatggagtactactcagctattaaaaagaatgaatttatgaaattcctaggcaaatggatggacctggagggcatcatcctgagtgaggtaacacattcacaaaggaactcacacaatatgtactcactgataagtggatattagccccaaacctaggatacccaagatataagatacaatttgctaaacacagaagagactttaagaatgggaggaggtattagaggagacaaggtttcctgaaatcttgaacttcatcattgcctacaagtactgttttctttgaaatgattttgtttgatttgagactatttgttaattatgtaagtctggtaacaacagttttcagtcatgtattttcctttgaggcaattactgtgttgtatgcagtaggtatacgtctggatactttgcattttcttgtatagacaaagatataatttcaggagctaggtgtaattgatcatttttactgtttttatctcagtattttacagaattgcatggcagtgaacacactagattcctagtagtttgatacctctggtcaggtgtgtgtatcagagtgtgtagagtgttccttcaatgtcagcacaagaggagttcttactttgaactggatacttctttattcctgagcagcagcatcccagcctgttcatcaacatcttccattcttcatgctacaccacctgttttgtttgccagtgtgtgggtgggggaggggagtattacttagagtgtatcacagatccctttgagaattcatgaaaaacactaaaagtgttacttttgctcttgttgctaatagacttttgtactcgatatagaagaattgcctatacagaatttaggtactctagagtagctgacattagtcagtgatggggtgatgaggggaggtgggtcgtaaatgagatggaaatatctaagatttcgtgaggcacacctgcaaaggtatagaattggttccttttattttcctgttgtttttttctgttagagtaaattggagaaagtacaggtgacagtgaaagataaactctagtgtgcatacaggatgcatgaagatacatcacacatctttcttagttcaaaagactgtagtagaaaattttcacataaaaattgtactaaagtggttaagtacagtttgggtttatgtaattactggaatggaattcttttggagagactaacattttacttaagattcaaagttagccttttcagtattgatcatcacatttgacttaagtgctcatctgctagtggtgatctgtaatgagattttatgtatttaatcttggaagatgtcctttcacagttagagccaccaaatattaacagtagtctataagcatatgattttaattgagcatattcattacttagaacacatttatacaattatttagacatttgtcttttaacatgctgtgttacatatgattttcacagctgggtgaaagctgcttagctatacggtaaacccaattttaatgtggaaattgcttttgcacctaaggtctgaaggaggctgtaccttgaattaagaaacaatttgctgtagatgtatgagaataaagatctaacttattttgacttttgatagcatgggaaatgaaaatgctattgttaaaatgacatgcactatgtttgcaaactgtggtgggggagttgttaaagtttgaagttgaacttgttaagtagcattaagttctgggtagtgaggtcacacatatgtacctgtaactcagtagtctgaggtaggaagatcttgaagtcagtgtagattacatatccagaccctacctaaaactattctgtagtggtaatgattaagaataattttcatgggttgaaaagattagcagttatgaacacgtgctgttcttacacaagatctatatttggttcccagcacccaacttccagtaactccgatgctaagggatgccacacctctggtctccgtagtacttgcactcatatgctcataacctacacacagatacatttattcgcataaaataattttcattccgaaacctttattaagttaaaattactgccataatcacaaaactagaaatggttaagtttatgaggacaaaagaaattggttactgatactactggtttgataacatgataggttaaggtatttttctgcagagtaactatcataggtttttaaagagcctttacattttcacaaactttatagtttgtccaactaaggcctttccccctccccattttatttttaccaccattgggtctgctattagttatactgagttgttttttctcagcttctttgaaagcattcttgctgtagttccacactggctatcctagaggctaactttgcagtctcagtttgaacactagctcctcaaacagtttagagtgttagtaacaactgccaactaaccaggactatgaaaaaacactcaaagccagctttttaattgttgatattgtcctcagtttccttaattggctgatagtctttaaaattgtctcaagatatttttttaaagagcatcccaatcaaacattgacctaacattggtaaaagagcttacctcagttggctgacacttgaaaattcctttttgaattccaagtttgaggccagccagaactacgtggtgtgaccctgactcaaaaacaaaactaagcaaccaaacacatgaagaatgctaccagagctaccagtcatgccctttaggtaacaacagaaaggataatggcccatctgtcaccgaaactacgaaatcccaaaccgaatggcttttaaacagcaaaagaaaaagaaatacttagctagaattccaaatctagtaagaccatccttcaccggtaaaagcaagttagaactgagaaaattggttggttaaagggtctggaaagatggctcagtggctaagagcagggctgtagtgtgagtcccagcactcacatggcagcttgcaaacatccgagatctgagcttcgttggcatctgtaggaggcatttcaccaatgtgatacacatacatacatgcaggcaaaacactcactcgtacacatacaattttaaaatagagggaaaaggtaaggaggagcctggcatggtggcacaggcctatttttaaaatgtattgtgaggagagggacaggggatggatgagttcttgcaaagcaccagaaaagtggaatccaaaagatcaaaggccagcctgggttacatgagaccttgtttcaaaagcaaacagtaaatgaaagacaaccagtggaggtgcaaataaaatgagttggtgtccccaccccaagaaatgcaaaaaagaaagaaaaaggaaataaagagcagataggttagactggaaacaaatagtagagcgagaaagtctgacctgacttgtctttacaagtgaatgtaaattaaaaagaatctaatcaataccccaaaatcttgagcaaatttgtgtcaccatgtaacccattaatgtgtgttaataattacgatcttaaaaacatttgcatgttgaatctggctatgtataaaagatagcatgtcaaaaactaaccggcttgttttaggagtccaaattcactaatgctgagaatgtcagttaaggtaaatcatgttaactatatatatatataaagacttagtaaattagaaacaagcttatcttcaagataaggaacataaagatatagctgtcagacctgacatgagttgctgatgctatttcctggattcaggaacaacattacttctgttcctggaagttctggccaattcaacaaggaagagaaaataaacaaggattatatcttagtcactgttctgtggctgtgaagagactttatgaccaaggcaacttttggtttttcgagacatggtttctctgtatagccctggctgtcctggagctcactttgtagaccaggctggcctcgaactcagaaatccgcctgcctctgcctcccgagtgttgggattaaaggcgtgcgccaccacgcccggccctgcaacttttttttttttttttaattattttatatgagtacactgtagctgtcttcagacacacctgaagagggtatcagatctcattcattacagatggttgtgagccaccatgtggttgctgggaattgaactcaggacctctggaagaaggtcaaaagcaatcaaaagcaaaaatcaatctccaaccatccaagtgtctgggatccaactcatgatcttctgggctcctccaagagcttgggtcatttctccagccatgccctttgtagcacccgtgtcgtcctctaggctccagatgcctgtactccactgctgctgctcttggtggtcatctcatggtactggcatctccaaaacactgcatgaccccttcagtcctgggcggtcaattgcaactgaggctgcactttcaccaatggccttccatggcctctcacagtgccaagcctcagctgctctgctcaactccttcatgccttcaaaaccagtaccacctgggtgacccttacacattaccaagtccagccacagcacaaggtacaactttggctatatctggaacaaagccactgtgctctcagaaaacacttcccagaagatgtcacctcaatgatgctggtctcttcttaatcaccgctaatttcttagctccagctaaccagcatcaatagtcccagtaatgcaaagtttttgcttttgtagttctggtatcttgttaatcacagctgattcttcagccccagctaaccagaactacagaatcttcacaatcaaaacagcaatggccttgaaaagagtctttaattttccctttgaaatttcacaaaccagacctccatcttctgcagtgttctcaacattatcttccaagctcctacacaatatccgacagagctcttaacaaaggatggatcttcaagcccaaagttccaaagtcctccctcagtcctccccaaaacatggccaggttgtcacaggaataccccactatgctgataccaatttgtcttagtcagggtttctattcctgcacaaacatcatgaccaagaagcaagttggggaggaaagggtttatttggcttacacttccatactgctgttcatcaccaaaggaagtcgggactggaactcaagcaggtcagggagcaggagctgatgcagaggccatggagggatgttctttactggcttgcctcccctggcttgctcatcctgctctcttatagaacccaagactgccagcccagagatggtcccacccacaaggggcctttcccccttgatcactaattgagaaaatgccttacagttggatctcatggaggcgtttcctcaactgaagctcctttctctgtgataactccagctgtgtcaagttgacacaagttgccagtacagcaagaaagcataatgataaaatataggaccaaagcaaaactgacaccaagcaggatagactccaagttctgtatctgtgtctgatgtcaaaagtgttcttcagatgtccagttcctttcagttttgttgactgcaacacacctctctcctggactagttccacaatctgataacaactctccttggcaggtatcccatgactctgacctccagcatcttgggttcttcaacactgtccaggcttcactcacaccttcatgcagtctatacttgaagtctttgaagtctcctctgcaatgacattattccaaaactctttaattagcctcaggcagatgcttaggacaggggcaaaaagttgctacattctttgccaagttatcacaagaatgttctctgggtcagctgataaaactcttgtgctgtgctgacatcattcaaattagcatacttgggtgtgtggtatagttaaccattactatctttcaggctcctactagaatgttaagccccacctttcaactgctttcctaatccatatcctaaaagtctgcttctaccaacaagcggccggtcaagcttgtcacagcaataccactcctggtaacaacttcttagttattctccccttgctgtgtgagaccccagtgaccaagacaactttttttttctccctctagacagggtttctctttgtagcctggctgtcctgggactctctgtaaaccgtgccggcctggaactcactattcacctgcctgtggttcccaagtgctgggattaaaggcatgcactatcatgcctgactgcaaggcaacttttataaaagaaagcatttaattgtgggcttgtttacagtttgagaggtttactccaaggtcattatggtaggaagcagatagcatggtgctagagcagcagctgcgtggtggtggggtgggcacagtggactgagaactgagcttgggcttttggaacttgaaagcacagccctagcgacttgccttctccaacaaggacacacctccaatcctttcctaatagttcatcagctggggactaagcatgcaaggatttgagccctatagggcacattctcattcaaaccaccatggattagaaaaggattcaaacttttatagcaaatgattggccttaaaattaaaaaaaaaaactgtaaacttatttaataatatacaaagtcagtatacaaaattaattcctatatatattacaattacataatatcacatagtgggaaactaaaacatttatactagtcttctcaaaatcagcttgatgaaaaatactcaagactgccatagtaaatgcttcaagaggaaaataaaccttgttgaggaaaacaaccaaagcttgtaacatagcccagcaagtaaaggcacacacaggcatgcacacaagagaaattaaataaaagaataaatggctaataaaaggcaagggaaggtcatgggttagaagaagacttaaaattaagacatcagttctactagactcagttcatttcacaaatatcccacaagccctacagatttgggggtcaaacaaacacaggccaattttaaaactttgggaaacataaagatctagattaccctaagaatgttaaagatcagaattgaagtgttcttaatccatgtattgaggctttacaaaaaactatgatggtgtgatgatgtaaagagactcaggcactaagaagggtggtggttgttttataagctgccgatgctgtctaaggagggaaaggatcacctacaacacgtgctataactctgggctgaagagatggctcagcaattaagggtcttccagaggccctacgtacccatataatagctcacaactgtctgtaactctaatttttttggatctaacatctgtgtctttcaccagcactgcatacatgggtatatagacatatatgcaggcaaaacacctatacacataaaataatcttaaaaaacaaattgacttttacttcaagcaatataacaacctaacttatgatatacaattaattaaccaaatgtcaaaaaaaaaaaaaaaagaaaaagaaatgctcttagcaagggtagtggtttcttggtaagatgtaaaaagctctaagaagcgaagacaagattaacctagttgaaattgttttaaaatttatgttcattaaaaaacttctttttgttgaaacagtattaagagaaggggaaaggtccatcgcgctttaaagaataaataagcaatatttaaatggcccactcattataggaaaaggcactcaaaaccttagtgatcagggaaatgcatgtcagtaaatgttgctacatagccaggagagccattaaaatgtaaaatagggataatagctagtgttggcaaagttacagagcagcatgatggtttattataaattgatacaatcactacgtaaactgctggtgtctcttaagtaaaagccttctctgtgactttgttctactcagtgttaaggcataggagaaatgtacttgaagttcttgataacattgatggtagcaccaaagtgacccgtccttgagtggagtactgcaggtgaccgtacagttacctgggtagtaggtgtcacagaaaagattatgagcaaaagagtttatttctgttttctaaaattcaaaaacaggtcagattattctgtagtgaaggaatcactacacttggacattgttatttagggcaaaaaggaataattgtgactgcagggaacagcatatcctgagctgagaagtagcatacttgggtgtgtggtatagttaaccattcatagagttatatatttgggcgtagttatgtatacagtaccttagaatgtcttttgtttagctatagtatagtagatgtttgctatattttgattgtacttagtggctttattgcagactgaaggattttaccatgaaattttatctataaagtgaagaaagtactggttgtgacatgttctgaattgttcctgttgttttttgctttctaagtgttgtgaaatccaagtgttgtgatcctaagttatcaaatctcttattgtatagtgagataacttgggctttctgggataccaaaagggccaaatgacaggagatgatagtgttctgagcctaatgaggtttgcagccttgactgtatctttataatgtgatgtgaccttacatctgaacctttaattcaaacactggagtcccgtgaatattgtgggctgtgacaggaacagatgatttctttatggtacctccacccctccaaaggtgactgagaaatgtgacccaagagtactctttgacctgagaggtcttcagggtatcattctgaactggaagaattgagctgctcaaggactgccttggttctctaacatttggccaattcactttgagatttgatcacttgctaaaggcctttgaagcctttagtttctgatagcatttcatcagtccctaagatctacttcagtcgaaaatgtacctttatgtgctactaggtggggagaaaaattaatcctaagaagctgttggttagaaatacaacttcagctggcgtggtgcgcgcctttaatcccagcatttgggaggcagaggcaggcagatttctgagttcgaggccagcctggtctacagagtgagtcccaggacagcctgggacacagagaaaccctgtctcgaaaaaccaaaaagaaagcaagaaagaaatacaacttcagccaccatcattgcaacatgattttatttgatttttagttttgtagattaatgcattttaaactttgtaaatatgttaactgtttagaattacttgggggttcaaaattaaacctatagctggggaaatgggtctgcagataaaagttctttgcccaacatggagaccaccatgtggttgctgggatttgaattcaggaccttcagaagagcagtcactgaaccatctctccagctccagagtccttttcacctcaagcacatgccattgtgccaactttaaatgtttttgtggtaaccaaaagtgcttttgttggcttcattgctactatttttaacaactaatacacagtgggcttccaagtacttagtgaattgatgacttctgaacaatcatttttagttctgcatttacttgaccagaaacttttatctttctcagtgcatacttttcccacttaaactaaaagtttaccaaattaaaactaatgtcaaacctagtatggtgccacacaccttgggagacagagacaagtagatctctgagctcaaagtcagccagagctacacagtgaagcagtaaaaaatacatacatacatacatgcatgcatgcatacaggcaaacaaaatccaaatgccaaaaccaggtgtagtggtagatgcctgtaagagctagggctcttgggagatagaagttagaggatccagagttcaaggccatttcttgctacacagtgagttcagactcagcccagtctatatgagatcttgtcttaagcaaacaagggagtgtgtgtgtgtgtgtgtgtgtgtgtgtgtgtgtgttggtccttccttccttgcttgaacacattgaatcaagggcatgtgtatgcaagtgtgaggacagggacagcctcttggtgtcattccttagttgcctccactttaattttgtcttttgagacagtctctcactggcctaagaattcacccttgaattcaaagtaacaaacaatcagcaaaactacttttttaaaaatacaaacccaacacagctttaaatgaatgctcagattacagttgtgaaaaacttgggtttttctaagcagtttaggctggctggccagggagagccccagggctctacctcctgtttctgtcttcctggtgctgggatttcttcaccctgctttttaacatgggtttttatgcttttaagatagtattattagctggcatatttgccaaggtacatattaaatgacccacattcatcttttgatttaattataattgccattaagtttcaacttatgaacaatgttgtattcttttatctttcttgctagtttgatgttaaatgtagtcacagaatataatacaattttaatacgggtgcctgggttgacttttggatttttgatacaggctttttctgaagttcattctgtagaccaggctggccgaaaaatcatggaatagagatccagctgcctctgtctcttgagtggtgggatcaagggtgtgtgccaccactgcccagcctgactttttaagcctgtttcagtctgacaaatcatagcagttactggggggaggggaggaagggattgaggtgggtaaagaagggggggaatcatttataagtggcaaatagcaggggtggcatgttgtagtgtttattttaatcaacaactagacatgtggtcggtaggcttaggtgctgctgatacttgatggagctgtttgaagctggatcatctcactctgttcatggctgctggctccctagtgatgctgttcttcctgctcagtgcaaggcttgatctaatgggagtaagctaggccttcagagatgttagccttgaagtctattaagtatcttggtctactagctcatgctgtgtccttggaaaccagcaacattgggaattaatatcacagttcataaaataaactcattgttagccatacaacccagtccatgtgtgatttttttttccttttttttttttttttgtcttcaaaatatggatgtagctactatctttaagcatctttattttatggataattttgcaaagctgatgagcttcctcattgtttttaccagtacgaggatgtcgtccaggaaagattgtacaaatgactgaagcagaagtccgagggttgtgtatcaagtctcgtgaaatctttctcagccagcctattcttttggaattggaagcaccactgaagatttgtggtatgtaaatggggaaagttaaaaatgagttttccaaagcacattttctcctgtatatatagtaagaatgacttagtgaaaacttctaaatttgataggtttattaaattggaatccattggaattcataataatgataggttagaagcaacagaattgctatttgtagtatataaaatggccctggaaataaaaagaatagtgttaagaaaaatgggcagattctaggagattgctaaaatcagtggactgcagcttaaaacacatccctagctcatcaaattacaaacaaaaagtgccagagattgatatttagtgcttttaaggatgtgatgaaattggcattctagaatgctgcttagaaacagttaatagggccaggcagtggtggcgtacgcctttaatcccagtactccagaggtgcaggcaggcagatctctgagttcaaggccagcctggactacagatctgagttctagggtagcaaggactgtacagaaaaacctgcctggaaaaacaagaggaaagaagaaagagtcatggaatcttgagtgtcagcgtggcaggatccatggtcttctagcttgtagtgattttcttttcttttcttctctcttcttttcttttcttttttcaagatttatttatttattatatgtaagtacactgtagctgtcttcagacgcaccagaagaggcaccagatcttgttatggatggttgtgagccaccatatggttgctgggatttgaactcaggaccttcagaagagcagttggtgctcttacccgctgagccatctctctagcccaaatgattttcaagatactacttagacgttgtgtttgttctcttaagtcttatagttgttttagcccaagacaaacacttagagagaaattattacattaaaagtagcatttttctgtagttcacgtacaacaggaagtggtataaatactacagcatgctggcgtttcctttggtgatacaggcagtctttgtaatggttaaagtgtgtctgaagaaacttctgtgcacttagaatagcttttgccatatgcaaaaatgtcctttttgcttgtttctaagcttgtttctaccttgaatccatataagatgtttggggcattttcttctaactgctcagaaatgttttttgctaatggggtactacttactgtcctttcccaggctgctgccctttccataccagggttccactgagctcagtatcataatgtcggggcaaaggcactgggaccctggcacctggtgtggaggagaaatttgtccctctctggggattgttttctggtctgttgttaggagcaaagtgggaagggacacattccatcaacactcaaagctccataatgtccctcagctggggttgcagggtagtggaggctaggagtatctcctgacctcagcacatcaggcttctaggatagtgtcagagaagcagctctgggcaaagtgacagggtttggggaaaagtcacaggggactgcagtaagtttggtcattatgtaggtcgagtcaggagaaataggtgaccatgttcttcttggacaaggacgacttgtggaagggctccctagcgtccaggctgagcgttatcccagtggttgcctacttgttccaacttggagggcatcctcattccagtggctattgtagaatcattgtggttagattgagggtactgcagatttaacatacatctattgttctttgtattctctttctctgtggggtttattaagtcatgtgtggtgccttgtacagtatccgtcaaataaataggtgcaaagtagatgttggttagtgtgcttagaacatcacccttaaagcaaaagcagtagtttgaaactttgtagaaatgttcagaatcttgtgattattataaataaaataataacattaagttgaaaatataataagccccaaaatatatttagaacactaatgctgaacattatcacttgacacatcacattgtaaaccatcggtattttacatctatgatcacatgactggtgggagttagagcttgctgctgccactcagcatctcccaagtgttgtactttcactagcatgggaaagtcagcggagctacagtgcttataaagtgagagtcttgtaaatgaaaccattttaagtcattgtttgtatttcttactagtaccccttcggtgttacttaatacagcaataatttgaggtaatagaagtattttagctcttgatatttggtataacaagtatgacttaagtttagctatcggctaataagagtaactgtgccagttgtctattattttattagaaattgcctgacatttttgtggggcacaacaatattagcttatcatcccagtttttataaggtagggattcagacacagctttgctagatcttcaggttctaagtcctagaagcataaaattgcaatcagttgttctaacctgacttcttttggttttaggagacattcatggacaatatacagacttactaagattatttgaatatggaggttttccaccagaagccaactatcttttcttaggagattatgtggacagaggaaagcagtctttggaaaccatctgtttgctattggcttacaaaatcaaatacccggagaacttctttcttctaagaggaaaccatgagtgtgctagcatcaatcgcatttatggattctacgatgagtgtaagtgggatttcaaacacaggagaggaatttccatatgcttgtacagttcagatgaatttcttcaacttagagaaactaggctttttgttttttaaaataggatctcagacaaagacccagaaagaagagcctaaagctaggaaacacattaaattgttttcttgggatctccttggtcatgttttcaggctggctacagcttagttgactgaaggaggcctcttttaattagtttatctggtaaaatctccaagtgaaacaaatacaggtcagtagtctccatttttctgctgtttgatagggttcgctttttacatttattcattattaatatgttttgtgtgtgtgtgtatgtgcatgcacacacatacagtgcatacatggtatcatgacacagatttggaggtcagaggacagcttatagttagtcctttccttgtaactagaagttggaagtagagaaatggctcagcagttaggagcccttgctggtcttcagaggccttcggtttggttcccagcacccacatcaggagactcacaactgcttttagctccaagttcagggaatcccattctctttatggtctgggcaaccacttcatgcacatggttaatacacagaggcaagtaggtacacatgcatacacataaatagaatgctaaataaaagtcaaactttattattaggattttaatctgtttatatacctagagtggttactgagtccttggttatatagtgttcttgtttgttaatcagtctgatttgctgctgtgtagcagtctttccagtgtcaactctgcatttggatttgggttttccgtttaattaaattaaatatgggaataggtgtatttagtcacctatagttaagtcatttctagtagttttcaaatcaaatttatttttgactgctaaattatctattagaatctattttttcttaaatatggtagtcttcaacaaactcatagcttaattcctgtgatgatataagaaatgtttgtaaatgtgttgtatatgctaatcacttctaacattggaatgtggtaaaattcaaggcaagttactttaaaaaaacagaatttatgttatactaggaatcattctgtatttgagttttacattacatttttgtgactttaaagtcaggcttgtgcagaggaagtaagttgaagtacatcaggtgatagccatctttctatgaggtatattagtatgttcttagaagataacttaaggcctgaatgtccctaagtgtagaaaggacatcagatggaacgatgtgtgggaaaactgcttgtaaacagtctataaattgtacttaatcattaaaaggtagaataggacataaaacatgaaattaatagttgtgactttcatcctttctccggcttggtagttgaacttaatgccaaatcaggtaactctcagtggaagctgcctttgttagagctgggctatgggaaaattatagccttgtgtcttttcacttgccgtagtgacaaggtgtaatttgttctaaagtgaataccacttaggcacttggaagtgttcctttgctggcagtaggtttatacttcattactcagtgcattaagttaacgtggactagctccagaggtaccattttcacttatgtctacttacttagctaggaaatgtattgaatgggaagaaattagctaatattctattagatgatttcagaagtattatactatagtgatttttataagataaagttgtttgatgaatgtgtttcatagaacagtacagtctttgaagtttctgcaactctacagaaggtattgaatgttttattccccttttagaactatctaagaaaaaaaaaatacatgtggtgataaacctgaatgctggacaagtactcagatatgagctacatctccagtgcatgaaactgaacatagcttattcacttacagaaaaaggggctaagtaattgtcttagtcagggtctctattcctgcacaaacatcatgaccaagaagcaagttggggaggaaagggtttattcggcttacacttccatactgctgctcatcaccaaaggaagtcagatctggaactcaagcagggcaggaagcaggagctgatgcagaggccatggagggatgttctttactggcttgcctcccctggtttgctcagcctgctctcttatagaacccaagactgccagcccagagatggtcccacccacaaggggcctttcccccttgatcactaattgagaaaatgccttacagttggatctcatggaggcatttcctcaactgaagctcctttctctgtgataactccagctgtgtcaagttgacacaaaattagccagtacagtaatcaagcacagagggaaaaaaaacagttattgattctttaattctctgtgttttccctttttataggtaaacgaagatttaatattaaattgtggaagacattcactgattgttttaactgtctgcctatagctgctattgttgatgagaaaatcttctgttgtcatggaggtagagtaatacatttgctgtttagaagagaactgtaataatgacagtactgtgggaaagctgagtgtggcaggaggtgtagagcaactagaagccagcttgattccaggccaggcaggcttaggaacaccccgtctcaaaataggaaattcttcactgggccttacagagaatgcctcctatacaagtgttaacagaagagtggtccacacacactggaatcccaggactcagaggctgaggcagaaggatctcaaggccatcctgggccatagattctacctcagaaagaaaagagaagggaaagagaaaaggtaattagatgtgtgtggagtatttttactttgcctcaatttatgcttgaccagctggtcagatgatttcctgtggtgctgggatctagctcatacctcagattttaataaaaggtacttggccatcattaagaatgagaggctaattatttttctgtctgtattaactaccttttatcaaactctaaacataattcatacagctttccaagttttgtgcactacgccaacatgttaggaaatgaggacattagaatatagagattagaatttagatcaaaaacagttttcagctctccagctttagcacccagctttaccttggtgtgtttctcatttatgctctgaggatcataaattgccatgactaatgactttctaaagagcttcaaatgtgtagtagtcatgaacaatttagtcttctaaagatgaaagttttgtaagtttacacacatgagaactccctgaagaatatgcagtaaacttttctatgatagtacgtgtacacacacacacacacacacacacacacacaaaaagtttttcaaaagttagggaagattatggtaagggcttgctttgaaagtcctgtgacctaaattcaatccttaatcagggatcttgaggccaatgaggtttcttccttagaacaaggtagaagcaagtacacatttactctcagcacccaggaggcaggtaggtatagttctgagttcaaggccatcctggcctatgtaccaaggatcatgccagccagggctttatagtgagaatcagtctcaccacacacacgcatacatgagcacacatttacacacactatggaacaatttaatgatatacaggaaaaatggtttcttggtatcatacatggtagtgtcttttgggaagcatagaggtggaaatgctgaaagatggtggcgaggcctgagacgtgagtggtttattctgacagcacagttcactctattagttgaaatgttgttttccatagatactgaccattaaaggcattggcagagttgaggtgggaatatggcagtgtggaatatgtgtatttagcttcatttcttgtggtacttgaattacctaccagagaaataataccttatgttctactttacagatatattcttgtgcttttttttatccttctgattattagtttaatgcacatttttgcagataatgagactgttgaggtgggatatggggaaaggttcctaggggaccaagagaggcgtataacttgcaagtaaaaatgctttatggggatctggtggtcatgaaggtacagaagcagtaggaaggttgcaactaagtgtgttctcaggaagggggagggcaactgcaaaaaagtagtagagtttctccttggaatctctcgaagaataatgatttatttccttggtagaaagatagaaaagatagcatgagtataattctgctagatagcaagtctctttctttctatatataggactgtcaccagacctacaatctatggaacagattcggagaattatgagacccactgacgtacctgatacaggttagtatatacagaagttcagttcattgctttacataagatgttgacgtgcttggacctaattatatttagtgaaagtcacttagaaacctagtttcatccctgaaaacagttaacaaagtgatcagagcaagaaaaggagccgccctgccagtcttaattaaagtcattaatgtgttcatgtcttcgtgataaatcagtaaagtattgcaatataggctggtgctcagtagtagtgtaatgcaaggagtggataagctgtgtacaggaaatgctagccagaaaggaaataaaggcctccacgtattacatatacttgagtatgccacacacatatttcctgtatgcagaagtcaacagaaagtactaccctaggaagctagagaaagttgtactttaacaaagactggtttagactggagtgcctagggacaagggatgatcattgaagtttaagaaggcaggagagatgtgctgtaaatgtgtgctgtattctgtgcttgcttctatttcactgttggtttgtttgttttttttctgttttctaaaggtttgctttgtgacttactgtggtccgacccagataaggatgtgcaaggctggggagaaaatgaccgtggtgtttcttttacttttggagctgatgtagtcagtaaatttctgaatcgtcatgatttagacttgatttgtcgagctcatcaggtatacagtatacgatattttattgttaatttcacacattttcatttattggtaggtaggtagataatttaccctgatgtcttttagtttctgtgacacttgaccaaggcagcttataaaagaaagaatttaattttgagctcacagattctgtgaccattgtggcatggaaaggggtggcaggcaggtaggcatggctctggagcagtagctgacaatgtccatcttgaggcaactgccatggggcagagagcgagaactaagaatggcttcggcttttgaagtctcaaaaatcccaccctaatacttcctaacagttccaccttctgaggaccaaacattcaaatatatgaactgataacctagggaaggtagtgggggaagggggttagagagttggctcagtgatttaatagcgctggcttttcttctagaggacccaggtttgattctcagcatattatgtgtcagctcaaaactgtctgaaactccagtcccaggagaccagacacctctagcctccaatagcaccaggcatgcaagccatgcagacacacttgtagcaaaacacccatacacataaaataattaactacaaacttaaaaggtaatatgacttatatgtgaagaacatagatgaggaggcaagactctagatctcgcaagtcagtaccaggcagtgttagcttaggcatctcactctgagtgattatgtttcaaaagagaacattctgtagtacagtgagctaactgcagccccacagtgctttgctggtttccaggtggtctgttctctttgtggcctctgtgtgatatggtaataatctgatactacccaaatgtgtcagtattgtttcttacaaaaatgaactgtgatatgagaatttttctgagacagttgtggaatgcaattataaactggttaataaatattttagcttaaaaaaaaagtcattattgatagtgtaaaagagtgtccactgttcaaagtgttcatgatccgtaaaccataattggtttataatatattggctgaaaatattttttacctcttattttaattttgtttttaaagtgtgtattttattttctatgtgtaaatgctttgctttcatgtatgtgcaggcactataggcttgcctagtgcctacagcggtcagatgaacgtgccagatctcctggaacctagagtttcagatggttgtgagctagtgtgagtgtttgagaactgaattcaagtccatctgagagagctacaggtgctcttaactgctgagctgcgtcttcagctctgttggcctctcttctttgttggtttggttttgtagattaaaaaaagttccatgtgtgtatgtggattgtgtgcaagtggaagattgattttgtttcgttttctaccatattgggaataaggccatggtctcagcacctgatactgagaggctttgttgttgtctggccgtcgcttcattagcctgcattagcatatgtggttcaaatgactcactaataagtgcttttcttattttctttgacaggtggtggaagacggatatgaattttttgctaaacgacagttggtaaccttattttctgccccaaattactgtggcgagtttgacaatgctggtggtatgatgagtgtggatgagactttgatgtgttcattccaggtaagacaggaaatgaaaatgtctgtctgtcttcccttcccttctttccttcccctttctcttttctttgagataggatctcccactctagcctgggctgaccttgaatttgcacttgctgctttcagaaactttaggttgcaccattcttatttcagatgtttaaactattaatccttctgttatatactggacagtgaataattactaaaggaatggaaatggcaagctggaaaggtggctcagtgagtaaagtgtttgctgtggaaggttgcagacctgagtctggatcttcagtacccatggagcagtggcaagtgcctgtaatgtcaggctaaggaggtagagacaggagggtaccagggtgttgctgcctagccaccttaacccgttaatggacttcaggtttatcaaagcagctgaggaagacacccaacattggctgacctttctgtcatacacaggtatcctcctacacagaaaagaatgaatgcagtggataaaataggtttctgtttccttttgcaagtttagtgtatgagataggcattggggaagaaagagatgaaaataagataagagtgtttacctaaaagggggaatttccagtcatctttagtacacttacagaaacatgcacatttcaaaggattcgctcttgctttgttgtgctgcttgttcaaaaccattcttaattgctggttggtaaagctcacttctgccacatgtacacattgggattgcattcagaactctcattacctcagaactctctgaggattcatacattcctcatataaaatggcacagtatttacatataacccatacatagccttcctttccgtaagatttaagtgatttctaaattgtttacaatgcctgctgtttaaattagaatcatgttgttggcctggtggtagtaatgcattttaatcccagtacttgggaggcagagacaggcagatatgtatgagctcaaggtcagctggatctacagagtgggttccaggacagccatggctatatagagaaccctgaggggaaaaaacaacaaaaaacaaaaccaaaaaaaattcatgatgtttagagaaggccagaaatgaccatagacgttcaatccagaaacttctttttctgaattttactgtaatatagttcaatccaacagctgaactaagaggttgcagaccactggtgtgtacaactgtcatcaaggtttcccttgtattaaactatatccatcaactacagttgaatgtcaaattgttggtatcttcattatatattaagggttctaaccaggtcttgtccttcagcagttgtcataaggagtcagaataatgttatttcttttacttggatactctagtatgtagagtagttttaaggaactagagagatagcttagcagctaaaaccactagctgctcttcattcagcagacagatgttcaattcccaacacccacatggcagctaacaattgtaactccagttcctgggaatccaacaccttcacacagacataaatgtaggcaaaacacccgtgcacataaagttaaaaataaattattttttttaaactacataatttttccatacttttttaataagtaaaactcttatttgtgctcactataattagtgtaataaaagtgtcaactgtttatgcatgatgtggttgcatatgtctttaatcccagcagtcaggaaacagagacaggcacttatctttgagttcaaggcttacctgcattatatagcaagtttgaagtcaacaagatgacatactcagaccctgtctttaagaaacaaaatgtagccaggcagaggcaggcggatttctgagttcaaggccagcctggtctacaaagtgagttccaggacagccagggctaaaataaaaaacaaacaaacaaaaaagaagcaaaatgttgcaagggcgtggtggaacagtttaaaacttaatactaatgatggaaatgcaaattactgcagctgccttggaacacagtttggcagttaattaaagggttaaacattgtgtcattagatgactgagcacaattagatctatacaaaatcttttataacattatttataataggtaaaagtggaaacaatcccagagcctagctgaagaattgataaataaaatgggctatccacccatacaatagaacattttagcaataaaagcggctacagatcctgacagatagcgcagcatggaagaacctattcacacagatcatgttatttgctaacagtattaatgatgctaatagttggcatcagttatatgaaatattgtagactagacaagtccagaaggacataaggtgggtgagtgattgctaacgcttacgaggaaggagatggagagccagaatgtcatctgactaagaacaaggagttttgggggtttggacacaaactactaaaatcagataactgtagtaaatatatactatactaagtaagtaaataaacatactaaaaactgtacatgttaaatggttaaacatgatgatatggagtgggccatggtggtgcatgcctttgatcccagcagtcgggagcagatctctgagttcaaagctagcatggtttacagagtgaattttaggacagccaggactacacagaggaaccttgttgccggggcaggggtcaggggtagggaggaggtgtgtaggggagacttagtcttgtggaatataccataaaaaaactttaagtaagacttacctgtatatgaaatatacagtggcttacgcctataatctcaacattcaaaatggtgaggcagaaggattgccacaaatttgaggttggcctgactacaccaacagcgtatctcaaaaaacctgggggaaaggtcagatttagatgagaacctagggtttttgactcagaccagtattttttcctctctaataaggtgaactagtagagaagattctttaaaacttaaatttaagcctcagttatgaggctgtctatctaagctgtgtttctagaatctaaagcaaatgttaagcaagtgttcttccttgggggagggcggtgctgcagaccaaatccaggcacttcctattgagctgctcggccaaacttacacttcaatctaagaaatgtttgtgcagaattgctaaggtatttttattttcactggacagttgatggcatttattgaaagaataatattgttaataaacacattatttatttatttatttaaagatattgaaaccatctgaaaagaaagctaagtaccagtatggtgggctgaattctggacgtcctgtcactccgcctcgaacagctaatccaccgaagaaaaggtgaagacaggaattctagaaagagaaaccatcagatttgttaaggacatacttcataatatataagtgtgcactgtaaaaccatccagccattcgacaccctttatgatgtcacacctttaacttaaggagacggtaaaggatcttaaatttttttctaatagaaagatgtgctacactgtattgtaataagtatactctgttataatattcaacaaaattaaatccaaattcaaaagtatccattaaagttctatcttctcatatcacagtttttaaagttgaaaagcatcccagttaaactagccctgttagtgacccagatgaaagcatgaagatccatctgtgtaatgtggttttagtggtgcttggttgtttcattattttgagcttgttttgttttgtttgtttttgctagaataatggcatctacttttcctatttttccctaaacatttttaaaagtgaaaatgggaagagctttaaagacattcaccaactattcttttcctttacttatctacttaagtaactgttggatcttactaagaaaacttacccctcattacagtaaaaaggaactttagaggtcgataggttttaaaaatatacaaactatctgatccattgattttaatcaaacagtttgactgggcaaactttgcagctgataatgagtatttcgctttttacaaaattgccactgatttggatttgtgcactctaatctttaatttattgatgctctattgtgcagtagcatttcatttaagataaggctcatatagtaatatccaaaactagttggtaatgtgattatgtggtactttggctttgggttctaattcgcacgaaacaccttttggcatgcttaactttctggtattaccctcacctgcattggttttgttttttggggtttttgttgtttgtttgtttttagattcacagaacatgagaatcctttttgacaagccttggatagctggctctcttctttccctctctctatgtgaaggatgtatttaaatgaacgctggtcagtgggacatttgtcagctctgaatattgggtgcttcactaataattgccatgtgaatgttgttttgactgtaaggctatgtcactaaagatttttactctgcgttttcataatcaaaggtcatgatgtgtatagacatgctttgtagtgaagtatagtagcaataatttctgtatgtgatcaagagtttattgcattatttctttccctgttctcttttttttttttttaagggttagcattaacaaatgtcaaggagtagcaaagtcaacaaagattttagaaggaggaggaactaagagcatacacagacttatgattctttggatgtgacacttattggatgtgattctaaagtcttttattgaacattgtcaaattcgtacgcttcataggatggacataatgtttatataatgcccttcttatgtgttaccatagatgtgtgaaaccttatagcgtccttgaaagtgttaaattgagaactctgttaacattttatggattgacacattatattactgcaagtaacatttgattttcagcacagtgcaaaagttctttaaaatgcatatgtctttttttttctaattccattttgtttaaagcacattttaaatgtagttttctcatttagtaaaagttgtctaattgatacaacgtctgagtgattattctgtgttgttttgttttacagtgagatatgtaagcacaagttgacatagactgaagcatagacagtctctgagctgtagccatgttctattaggtcacacatgcttttatttaatgcgattggataacttacatactagagtaaacgaacaattgtttactcaaacaattgctaataggattttagatgttatctctgagtaatcaatacttaaggtagctcaagaaaataagccttagtctcaatattagttaatgtacactaatttgtatcttaaactgttttgttttttgtaaatgttcattcaaattaaaactaggggcgaaaagtaagcaaattagtattggttgttaaaggatagacatttaccatgttggaaaattattcagacctcttaaaactactttacagcttctcatatataagtactcagtacatcatgtgctcctaagaatgataacacagattattaattatactagtttactgacaaagtcacaaagacaaacagtaaaatacaggctacctttaaccccatgattggtgaggcagacagagacaggtgaatctatgacttgaagcctagactacatgttacagtgtctgtttactagatgaaaagctacaagcaattgcagagaatttgggttcaattctttttttttttttttacacgtattttcctcaaattacatttccaatgctatctcaaaagtcccccacaccctgggttctattcttaacactcagaaggctgtctgtatggctaagccaaggggatctgctgttttatgccctctgagggcaccaggcacacacatggcacacatggtgggtacatagacatacatataggcaaaatcctcatacacataaaaataaaatgtttaaaggatttttttattaagtgttgagcaaataaaatgagttttttgattgga

SEQ ID NO: 12 - DNA, 20307 bp, Mus musculus
gtggtggccagaaaatcacagaagaagccagcccagagcctcaaggacacatcaaggtgacttcatttgtcccaggcccttcttaagccactcaggctgacaggcaatgacaactagcatatgttgatttaaaaaaattggaagaaggggttgggatggggtggccttgcatatcccctaatccttgcactttccggaggcagagcaggaggatctctgagagttggagtccagtctggggtatacagcaagtttctaaccaactagagctacaagtttcagagacaggtgttagaagctttgaggaccacacagaactcagatctctgcctgcctctgcttgccaaggggattaaggcatgcattgctacacttagcgagctgccacgtggatgctaggaattgaacctgggtcctctggaagactagacagtgttcttaaccactgagccgtctctccagcctcaattttttttttttgtttgtttttcagacagggttgctctgtgtagccctggctctcctggcactcactctgtagagcagagtggcctcgagctccaagagctacctgccacttcctcccaagtgctgggattaagggcatgggccaccaacgcactaaaaacctgttttgttcatatttcctaaagggagtggcatgtggctcagctggtaatgttcacctagcatgcccaaagccctgttttctgtctccagcatgacattcagcaggttcagggaccacacattggatttcaacattctgggggtgcaggcaagaggatcagtagttgaaggtcaccctctgatagctacaggttcctggacaaagttcaggctctctaatgtgagcttctttactcacaagagagccagttctctggggtgtggctatggactctggggcatggctatgaactctggagggaacatgtcccctgtatcatcagcttggtttgtaattttccttcacccctcaccacatggatgggatgacatacatacctgtcacttggcatctagataattatgacacaatgtgtgggcataaacattttaaatctttctattgcttccccaggagtgacacacaccctatgtgccaaagtctgctgggctccgaaggctccatttagggacttattaaacaaaaggctgttgtttcaggctctgcccagttctagtcctcctcctggaatttagcaactgtgtgaccttcagcaaacaacccagcctttccgtgcctaactttatctgtaaaatgggaacggtgggaatatatcatctctaggattgtatcggggattccatcagctgactttgtttcgttcgagacaagattttgctgttagccttgactggtctggaacttgcctcaaattctcaaagatctgcctacctctgcctccccagtcctggaaataaagtcctatctaaccatgcccagctacttcatcatattttattttatatgtacgtctgctagaaggtcagaggacaacctgtggtagcttcctctctctccattaggatgtggggcatggaactctaattggttggcaggcatggagggaatctcttgtctgcagagctgtttagatagcctcccacaaagagacagattttggctcatactagtaagagtggtgtgtgtttgtctctccatcccacaaaatgagtctcccgggggagagaaaggctttctctgtcttgctcaacctgctccacccagaatagacactccttgtgaatcagtcttacatatccctggccctatggtgtccctgggaagctaccctgccctggagggagtccaagattccaaaaactcagtgaagaccacattgacctccacagattcacagcagggcttagcatcgaccccaggatgtcaagaagcctggtgcaccagctgccttggggtgatgaatgaccatttgtgtaatggtgtgcaaaaccatagcttgggacaatgagttggtgagtgggcaaagtggagcagagggctgaggagagctggggagaatcctgaaaccccactgtcgggggctagcatcaggaaaagggctcagatctagatgggtgcatcacacaccatgctgcggggccacgggaagacagagcttgtagaaaatgagtctctctatggggagacaaccttgcaaggcattcatggaaaggaatcctcagatccatccctcttgctttgagaaacatgaactggaggtggggctgggggagaacattttgtaaattttctggcgtgctactgggaagagataaatgggtcagatgggtgtgcatttttcttccataggcagctagcctgcgaggcagcagagttggggacccctcaggtgaaggggtgcccctggggagtcaaggaagctagaggcaagcagcagatgaatgcgctgaggcccagagcgcacgcacagcaacctcccaggaaatgtgcaaattcccattttatctgctccggcacgctcattcaacccagctccggctaaatacaggggaaatctcttaaattattatataactttctgagttcaagaaagataattcaaaagataggtagtagcaacccttacagaattctcccaacctctctctaaaatgtaaaagagcattagcgcacacaagaagctcacacaaattacttaatgtgccactcacagtggattaacatttttttggcttatcttgggggcttcttttttccaaatattgtatcgtagatgctttattgcagtattatgaaattttgccagtgctgggaattgaacccaaggtctcacacatactcgacaagcattctcaccacggagccgtatatctagctcttcttgttcaccaatgtgccagagtatttgaatccccaacgttccaacctcctcctctttatgattctccacaggcaagcaaccccaccccagattggctccgaggggtgattcagtgttgaactcccaccccgccatgtcggtcagacttctgtccccaagcagcagagccggaaggagctggtgagcctcgcagcgcgcagcagactgacaaagtccaccttcaacttgcgcgcgccaacttcacaacttccctctcccgccacagggggtctatttcccccgcccgtttggcggaaggatcccgcgcgcggtggcggagtcgcgctcaaccgtctccgcccgggctccgccccacccccgccctacgcgctcaaaccccgccccttcccggcgccggccccgcccccttcccgcgcgctctatgggcccgtaaagaagtcccggccgggccgctgcactcccgcgcgcgcatccgtgcgccgcccgaggctggcctgaggagtcggcggccatcttgttcttctcgtggttcccagtggcgagaggaggaggaagcccggagcggagcggggcggctggggggggtggacccgccgcggctgctgctgccaccgccgccgccgccaccaccgctcgtggggctcgtggcgtgaggaaggaggacgagtgagaccccggggcgagcgggcggcggcgccgctgctgctgctgctgctgcgggagggtcggcggcgggacggcgatggcggatatcgacaaactcaacatcgacagcatcatccaacggctgctggaaggtgagcggcgcgcgggtgggtggcggggcaggcccggccggtgggggatcaggcccagtggaggggccggtcttttctcggaagccgctttggatctggggcgcgcatacaagcgcctccgggtttggggaccccattattttttatcactcccctgtccttgctttcacatttatcatctggagtccaaggactcatcagagggacttggcttggcaggacgacgtcccagtgcgatgagagtccgtctcctgcattttcctaccccctccaccctcccctcaaagccatccatcctccagcgttctttcccccttgaacgtgcatccgcacacggacccagtccgggtgacagtgtggctctctctgatcatcagattgttgtgtccctgaggacagggggacgctgccgcgttcagcgagcacccgccaggtaggtgctgtgttgttattatcctggaagtgggtttcgcaagagaaactcccaagacctcgctggaccaagaattaacagttaaaagtcaggtttcaaaccccggggtagctaggaacctagagagaacctgccgccttccaactgttcaccccgtgggaacccaggatgtccagtggttgaggagaggttcaaaaacaaaaacccagctttccttttgtccgacttgggggtttctgccgtgtggtttcctactttggagatttcaaatttgacataggtctggatcctgctaatcacagatcccacaacatccgctgaagctaaagaggagaagatccagtaaccttgatttgggctcttgacactagcttgctttggcccaaaatacttttggagtggtggacagctgcgcaattctggtggtagatgtcttcaagtaaatcactgacagaaaagggaaaaggtgtctttttaaaatctgggcatttccccggggggcgggggggtgtttttcaagacagggtttctcagtatggtcctggggctgtcctggaactcactctgtagagcaggctggcctcaaactcagaaatccgcctgcctctgcctccctagggcacggattaaaggcttgggccaccactgcccagagcattgcctcctctttaatgttttgtttacatagggtttcttggctttggtttcattaaaacatggtctctccagtcgtggttatcctggcactctttgtgtagaccaggctagcctcaaactcacagatccttcttcctctccctccccaattaaaggcatttttattaccatgcctccttttattacagggtctctctcaggggccagagctgagttaaccttctgatccttctgcttcgtctcacatactagattttataggccactcagcctgacttatttttttcctatgcagtgtgtgcatacatacagaccaaagcacacgtgaggaggttagagggtagttgggcgtctttgtgtggattctggggatcaatctcaggtcaggcttgcaaagcagattttctactgactaaatcatctcaccagcttccctttctccctcttttaattatttttttaaattttctgtatgtattttgtttgtgtgcatgtgcgcgcacatgcatgcaccgtacgcatgtcttacccttatgaatgtctgaaaagggcattgggtccttgggtattggagttgcagatagttgttagctgtcacatatgggtgctggaaatggagcccagtttatctccaagaacatcaagcactcttagcggctgaaccatcacttcactcccccagcacccccttttcttgttggtttttttttttttttttttgtctttgtttttcgagacagggtttctctgtatactctgtatacagagtttctctggaactcactttgtagaccaggctggtctcgaactcagaaatccgccttccgagtgtgggattaaaggcgtgcaccaccacgcccggcttcagcaccccctttttaaatgagacagtttcattatgtagcccaggctggtctctgtctcaactatccagagtactgggattaaaaactttttaccacccctgtcttctcatagtttcttaatagtttttaagttctgagactgggtagcattgtgttagcaaaggctggcctggattttcagagatctatctgcctctgtctcctgagtgctggcattcaaagctgaactaccatgtttggcctggtttattcttttctgttgaacttaacagagtaagccactgtttaaacacgttgagaatgactggctgggtggtatatgtatttgtttctaacttatggacttgcttgacggaagccacaggatctgctcaggatgcgtatagttctcaatcagaggacgctggcattggggttgtgatgatttcaaaacctttaaatctagagatagctcagccggttttggctcttgcccttaatcctgaagtcctgatttgatcctctggattgacatgagattcaggagagaaccagactttgagggttgtctccaccccacccccaacagctccagcaagcatggccttgcacacatgcatataacagaatggtataaaaaaaataagggtttaattctgaaagggggagataagatttgtcccttggagccgggtgcctttaatcccggcacttgggaggcagaggcaggccggtttctgagttcgaggccagcctggtctacaaagtgagttctagaacagccagggatacacacagaaaccctgtctccaaaaacaaacaaacaaaaaactggaaagctgtcttagggctaagagtacacagtactcatctagaggatccaagctcagcttccagcactcatcccaggtagcccagtgcttgctactccagtaccaaggagtcagacaccagcttctcaaggcacacacatacatggctaacccacacagacatgattaaaaatacattttaaactgacagtggtggcacatgcctttaatcccagcactgagaaacagagacaacttaatctctgagttcaaggccagtctggcttacagaagaagtgccagactatccaacgctagctactgtagtccctggcacttaggtgggggcaggaggtcgaggcagtagcataatgaaatgagttcctaagggtctagcctgaactacagattctgtgtaaacaggcaggttgggttgggttcctaggcactgcggttctgtggcactctgaatgcagtgggtttccctgagtgtcaccctgggtggtcagccacactggcggaagatctgtgttagtcctcagcacagtgttagggacatgctagagcagaaaatagactagctgtgtgtgaaggtagcaccctgctctgctgttttagtggaaaatgggaattggggtgctttcttcatgaaacctctagtgttttttcttgagtagcaagtggttttaatttctccacatggcaatgtgcaagaacatgtttctgtaaatgaaggtcattgtcagcgcatggtgggaagtagctctgctaatacagattgctctccagtcctcgtctctgaaggcatctgactgccctaccgttctctaaggacttgtgtatgtgtcttttcggtgctcctgagggccagggtcctaaatgaaagacaggtgagggtgatgggcttgtgggtctggaagcagatcctagactgagcagagggcacacaacacagcaagatcatcattaacgggcatcctgccctccctccacaccaacaggtatatgtccccatactttacctggcaggcagggcaggaccctctcctcaatctgtagatgtttgagtcttaaaaaaacttaatttgggtgagacatcggtggcccaaacctttaatcccaggactttggaggcagagtgatgtctgagtttgagaatagcatgatctatagccaggcatagtggtgcctttaatcccagcactcaggagacagaggcaggggcaggcggatttctgagttcgaggccagcctggtctacagagtgagttccaggacagccagggctatacagagaaaccctgtctccaaaggaaaaaaaaaaaagagagaatagcatgatctacagagatagttccaggacagctagggctacacagagaaaccatctccttcctcctccaccctccaaaaacctccctttgtcaggtatggctcctagagccttcactgccccttcacttttgcaaggccaggctgcaggtcagtcctaggcaggtgggacacacaggacagccacctctcaaaggaaagcagctggcctgtgtgatcccacagtggctgggttggggtggtcagtgcactttaatatctgagggaaaagagctatgtgtgttttttactgctttgattcaggctgggtcacaggaagaaaggttgggtgggaactgcctcatagctgaggaagccccacaggcccactgaagtaaagcaaatcccggttccctgagctagaattctgggagggatttttgtcttcagtcttggtcacagttgtatcgggagtgtgaaggcaaggtactgattctgatgatgtttcccacctctgtgtcagggttgctgacatccttgtttttgaagggtgtattttaggacttacaggaagtagaagctcatggggtccaatgtgcaaggcactgatgctgagatgcggcattcagctgtggaaagattctcaggctgccctccaacttccttcacagctgagatgaccttgaactctgctgatcctcctgcctcccggtgaaatacagtgttcaaatggcttagtgttcattatagaaaacaagatacgtccttgggacgttttttggttggagcttttttagccacatcctggttgcccacgtagaggagatcagtgttgtcatgcatgacagcagcagtgccccagcgaggtgggagggtagtgggagacagtggggagagccatgcacttttgctgttggggggaaaaaggagttgttgaggagtttctttagcttgcatgttttgtagaaaataattcagtttgcaagaaagtgttcagtgtggcgagtactaagtttgtttggtttgagatctttttctacatgtttttaaaataccttgaaggtagtctttcaccaaggaatgacttaaatttgttttttatttgttgaaaaaaaaggtctcatcactatgtagatcaggctggccttggactcagatctgcctacctggggctagatgtatgcttggccttggcagtggctgcttaggtaaggttgtggtcagacagcaagggctgctgactcgccccttctgtgcagctcttagctccaaatcacattgtaattcatctagggctttgtcctgtccttttattacttattttttaattgagatggagtttcctgtgcctcggctggccaggagggtctgtgtagatgaggaccttgaactcccgatcctcttggtacacagcccagttttaatgctggggcagaaccccagggcttggtgcattacgagccttgagggtgctaccaaccctgtggaggcttttctatccctcagtacatctccttggcttccttctgtcatgctgtcttgcttgttgctagactgtggaaagttactgctgcctagagaggtgctagggctgcagactaccagcagcagcagtagactaccagcagcagcagtaacctgattaagctcagggtttatgaactttggggtttgttgtcagtgagatttttagtgaaatttctctgtgtgctgatgttagtgatgttccaggtaagtgcagtatctacagaacagcctttgttgttagctgccagccgacctaaataaatgtcccttttctaggtacacacaagagcctcattgggaacagcaggctgacccagcaatagtaagagccagagaataaggttagatggacaggtcctgggtgttaattgtgggaaagtcatattgcttgatactgagagctaaagtagatcttgccaatttggcagctttaagacatacccaatgctcactggcaaaaaaggcttgtagtcctatgcttattttgacaaccactccccccctcccccccccaatgccctacccctgacttttgaatcaaggttttcttggggctggttggcaaccaggtgttgccagccattcaggaagtataatgtattcttggggaaggggacagcctcagttgaagagaggtctgggaagtcctgaagggtgagaagttgttgccagctgtggaatgtgctatgtcagtgaatgctacatagacagggtgttgttagctagagaagaggggtgaagctgcaggaggatcataaatttcagggttagactgggctacataatgagacttggtgtcaaacagttggttgtggtgacatacagctttaatcctattatcgaagtggcaggcagatccctgagttgaagaccggccagggctacataggaagaccttgtttcaaaaatcttttaaaaaaattgcactcaaactgtctaaaaatggcagtggatgttttcagaatcaaggaatattagcaacacccagctagctctctgcagtagaaccctgtatgtatgtaacaaagcaagcctgtgggctgtaccaggagaagccagatacgaagattatcaccaaagacacaagagtgggccactcgtgcacaaatgcagagctctcctgaagaaaattcctgttgagaccagaagcttccaggagttcccttctaaccaaaaataagcctcctgctgggcggcatgcccaggtttttggccggtgaggcaggaatcctcctgtctctcctccgagtgctgcttgtcttgtctggctccagtggcaaaggaaagcctttgttgcttagctgctgtcctgccttccctcaaagactcactgcagggctacctccatctcagaattggccatgcctgcttagcctaggctgcatagccccagaggggactgatgagaggctcggtggataaagtgtgctgcataagcctgacatctgagtttaattcctggaaccagcaaatgggaagaagagagccaactccacaaatttgtcctctgacctactcaccctcaaccccaacccccataaaatttaaggggaggactggggaagatagttcagcctgtaaaattgctgcagaaacaaggccctgtatttgaccccaccagaaacagtttggtttggtttatttgagacagggtttatctccatagccctggctgtcttggaactctccatgtagtgctggggttaaaggcatgtgtcactaccgccaggctcagaacctattttctaaggtgtctgttaccccagtactaggggatggagaggcgggatcctagtgtagccaaagaatgaaatagaaaaaaaaagacgagagtacacccgatgtcaattcccagtggccacatcttttgtcttacagcccagttatatttgggggggactgaagctgctcagaggggtccattccttgctggctccctgtcctatctcataatgttttttcctgctttgagggcccatccttttcttgtcttttccctgaaaaactaatgttgctttgcataccatttcatccctgatcagcttgttccttcggagaagcggggatgtctaaagtcgtacaggctgaagggcagcttccctgaggctctggtttccaagcatgggtacagctttgcagccttgagtggactctgcttggtccatagcaggcctctcttgggagtgccattgctctgctgctacagggtcaaaagctgtgtttgcctgtagacttttctgctcttacctagctgtcaggacagatgattttatgaccgagcctgcaattggacaaaatttaagctagtgagatcagtcagatttaggattctgctaaaagcaaatctgttgactggtttatgatagacagatgcagtcttaaatgggctgcttagcctgggtctgctgaactcaagtgaagtgcctgcgtcctttgtcttctgtacaaaagagcagagactcagattctagctcactgaaggtggtcaggcccctgtgccaggcagccttgcggtaagcagggcctgcggcagccgttccctgaagctgctgccacttgagagctgtggggagttcagatccttggcagttggagttgttcttcagtctgcataatgattatctgatctcgagtaaccggaacccacacagatgtctccactgctgtcaccctcgcctcctgaggagagtgggctgtggccagaccttaccgaggccgtgaacagagagggctcaggagctgccatgactggtgctaggctgggagttgttctctttggatgcctgggaggcctttttcttaataggcactgacttaatgctggtgtcctatttggatatgaatgccagaggagaacctcagggtgtgtcattaattcttcaagggtgatttggctttgggttttttttttttccccttttagctaggctttctcactgactcactaggtagattaggctgcctggttggctttccagatgtgtctgtttccctctccagcactgggattctagatgtgtgccatcaagcctggctccccatgccccactccctcttctgtgagttctggaacttaaacataagttctaatgcttgcaaggcagtcttttatcagctgggccatctatctagcttctaggacatttcttaaaattgatttatgtgtctggtggttgtgcctgcatgtatgttagtgcagtctcatcggaggccagaggggtattaatagatccctagaactagagttagagttgtgactgactgacacgttgctgtcggactgaacctgggttctctgcaggagcagtcagtgctcttaaccagtgagctcagcagcattttaaagtgaaatgcagacaacacgcagaaattagggaccatggagttgcacttggtcctgaaacatcattgctggagacatgtcctgaacatttctgaggacatacttggtgatatgactgtgacctttcccactgcaaacttgagtccattctaaaagtagagaggcctggaagcagaagggaccgtcttggtacagggcacgagtgtgtcctagggactctgaaacggccagatacgggcagggtcagggcaaccttggagctgctgtaaaggagcctaacatagcttgaagtatacaactgacattccagatctcagttgagaccatactggccagccagaaaactgatttcagttacatttggccaactcctatctaacaggtgtttattatgagaaaaaaaatattcaatatcacaccacgccacccttctcaagttaacattttttctaagattcatttttgttattgacgtgtctgtgcatttgagtgcaggcggcagccttggccagaagctacaagcagtaattagttagctgcttgccgtgggtgtagctgctcttagctgctgctgtcctgaattgaacatttccatgtgaggctgtgtcctgactgctgtttctctccacagtgagagggtccaagccaggcaagaatgtccagctccaggagaacgagatccgaggactctgcctgaagtctcgggagatcttcctcagtcagcctatccttttagaacttgaagcaccactcaagatatgtggtgagtgtgggactcccgttactcagctgggggcacactggtggcctttgatgttctctgaaggagcatgggcatgggggggtgcggtggggggcctggtctagtttggctcactctgctgtaggagcaggtacagaccgtcctctagagtcgcacagtctaggtgggtattgcatctctgactcagaataaactgtgtgtttagagcgaggagaccttggctttgaccttgcagaagttgaaaagccatggccaaggtgggtgtcttgaatgtaagtgttgtgagggggcccgtctcactggacagtgacaaaggatgtgttcacataaatccctgaagctgagctcaggggactgcagtacttggcaggaaacaggtgagacaggatactctttaccacaaataaattaattagttgtatctggggggagctctggacctgtgcatttaaattttacatggtcagttgaactaaatatgctacaaccgggaaattcgtgttggcggtgaacgttgtagatgccattgtcatatgagatgagtgtctgaggggcgtcttgccaaggttttgtctgtttgctgatttgtgacactgatgttctcacccttaggtgacatccacgggcagtactatgatttgctccgtctgtttgaatacggtggctttcctccagagagcaactatttgtttctcggggactatgtggacaggggcaagcagtccctggagacaatctgcctcttgctggcctacaaaatcaagtatccggagaacttctttcttctcagagggaaccacgagtgcgccagcatcaataggatctacggattttatgatgagtgtaagtactttgcctctgaggaaccagggctagaaggcagcgggtagcaggcctgggagttggagctgctgcagcatacctacccacgtcctggtttagggagagcagcctgacctgggtgctgatggggttggacggaaatagtcttgatggcacacggctttgctcttagtgcagaccccagagcttgctccatctgctctgcttttcacagctttggttagactggttctggatagagagagcatctgctgaaagcatgggtcagtggcttgtgagttgagagcagcaccacagttgcaggcagtgctgctctgctgtccagccttatatgtgggtggatcttgcagtgaacagcttggttttgaaatggatgttgtagttttcttgtagtgcagctttatttaggaaagggacaataacttttctggaagatgcataatggagaaaaaacgctttaaaaactacattttgatttagtgtgtctgtctttgctggagccagagtgtagagtcagctgtctccaccctgaccttggttgccaggcttgcacctttccttctcagccccgcgctggctggctggctggcttccttcctttccctttctttctttctttctttctttctttctttctttctttctttctttctttctttctttctttgttttttgttttggggtttctctgtgtatccctggctctcctggaactcactctgtagatcaagctggccttgaaatccacccacctctgccttctggagtgctaggattcaaggtgtgccacatcacctccaccctcccccttctcctttttgtttttcctcagtttttgtttgtttcgagacagggtttctctgtatagccctggctgtactggaactcactctgtagaccaggttggcctcgaactcagaaatccgcctgcctctgcctcccaagtgctgggattaaaggcatgcgctaccatggcccggctttgtttgtttttttatttaaagtatgtgagtacactgtggttgtcttcagatgccccagaagagggcatcagatcccattacagatggttgtgagccaccatgtggttggtgggaattgcactcaggacctctggaagagcagtcagtactcttaactgctgagccatctctccagcccccaattttattttaaaataatactttgtacatattctcatatttgaggttatctcattttacttttttttctgaagtgtagaccacaaccttggctccagttgcagcttcctgaccttggtgggggtggggggtgcccaccccacacagccctggattgtttagtagaactgcactttgaactcatcattaattaaaaatgattccaaagccttagaaagatttggtgtgggaaaaagaactggcttgggggagggctttcatggtttcacacaaacataaaatggcaggagttttgagactctgggctgtttccgccagaaaagggtttggactggattggcaggtgcttaagggagtccattggtttagcacttgctaatatggggcacacacacacacatgcagcagcttgtagttagggctgcccctgttttctagattgctagagcgagcttgagttcagcatggagtcttctcacctcagacaacacagagttttaatccagccctcagctaaggcatgagtgacattgggctcagcagagaatgagagggaccggatggattttggagtggagtcaaggccatctggcctcagtttccgcattgaagttcttactattagctattggtaattggaggtgttgactgtgttcattgaagtttccagaattatccagggaatctgtggcctccccagggctgaaatgcaagtttcctcggaaggatctgaatgctgggtgctggtggtgggtggtctgcccagcttagaaggcgtggcttaggaaggctgttgctaggtgccgctgaactctaaaaggcataccgtggccctttagatgtccctgtgttctctgttaaagaggacagagtcctagtgtagtggcaaactcaacatactgctactcaagtgtgagatggcccaggagcagaggctgccctgtggtgcgcagtccttggccagttagagaatgtaggaggggctacggcttatgaactgtgaactccaaccatagttttcatacagtggttttaaagggcacacgctctgacaccccgttataaccctggtgcttaagagggagtatattgagtgtagactgttaggaccaagcaccggatgtgcccagattgagctttctgattgcccagagtccctggccctcctgaagtcagggtggggctcctgtctggctgcagcagcttcacagctaaggcagctgaaggaagagctgacaacacaggaggacgtggcggccttcagaagtagaagttccctcaaggtcaaggttccacaaaccagatgaggtttcaagtagagtgggtgctgtgcgctaatgtggctggctgaggtgccctgtctggagctctggggacgcgtttggttctggatgagtcctgtaccccagccctgtgggcctgtgggtgcccccatgtttcagttgcttgaaaggtggccttagagagcaaaggtgaagagcagtagggcgactgtctgcgttctcacaggtaaaagaagatacaacattaagctgtggaaaacgttcacagactgttttaactgcttgccgatagcagccatcgtggacgagaagatattctgctgtcatggaggtatgtgacatggaggtgttgccttcacctaggcagctctaccccttctgaaaagtcagctgggcacggtgaccctggccaggccactgtggtgccaaggcttcttcacagtgatacagcccggtcatttggatcaagtgtgaggaagcaggggttataggttgggaagagaacgagactttaaatacaggacgagccctagtgccctcttctcagccatgtgtctgcctgaaccctgactgtgctcttcccaaaagctctgctccgttcctcagctgttctgtgtgtgaggtaataaaggcattgcaaagatgtggaggtttgggggtgattggaaatgaagcagctatccaaaaacccacataatgtattaaggcgtagtctctatgccctcttggatcgggtacttagagaacaagactgttagatctgttgagtgacatttggtggtccatggaccacaggctcggaaggagctgcctcacagctgagcattgtcgtgcctgtgcccggccattgcatctggtgatggtggcaccctctgtgttcccttcctgccttctattgggagaagtgtggattcagtggttgggtgttgcggggctctgtgttgaggtgactgtctctgaccagcacaccaacagcagtacagtacatttcgtgcattgtttcccccaaaaccaagttaaaaacccacaggtttcaccgcagcatggcagtgccagcctcctcagcaggaggggctggccacatgctcaggccaatgctgtctgcacttcagggggtttcctttctcgttgaataggtttatcaccagatcttcaatctatggagcagattcggcgaattatgagaccaactgatgtaccagatcaaggtcttctttgtgatcttttgtggtctgaccccgataaagatgtcttaggctggggtgaaaatgacagaggagtgtccttcacatttggtgcagaagtggttgcaaaatttctccataagcatgatttggatcttatatgtagagcccatcaggtactgactttgactttacatgtacacttagagcatgggtgtgagccgaggctccttgttgattttggtgggtaggtatggcttgccacacagtgtgcctccggggcgagcggcagcaggccgagctcagtggggaaggagaggggcagctcgtgccccagctgctcacactgacacggtctctccacaggtggttgaagatggctatgagttttttgcaaagaggcagttagtcactctgttttctgcacccaactactgtggcgagtttgacaatgcaggcgccatgatgagtgtggatgagaccctcatgtgttccttccaggtgagactgtggccacttttcatgtgcttgcacctctgctgagcaggctgccctttcttttttaggaatatttatcccatggaaaaagtgtgtgggctcttcctaaagaaaggaaaaataatacggaaccttttcccaggattgacgttaggtgtagttgaattctttaagtgggtggaagcaagcggtgtgttacagtgtcacttatagtcagtgcttttggtgagaagagggagttggggtgcctctggctggcctggaacacaccctgtagaccaggctggccatgaatgtaagagagatttacttgcctctgcctcccaagtgccaacacatcagcttgtgccatgatatcgtctcctgtgctggcccctggttcttacatttcctccctatagtgggcttgatccatttcaagctcacgcagttgttaatgttactgtagctgtggaagtcccgaggcaggcctcagcactccccagagactgcagactccagtgccttccacagagcttcaaggttgtgggaagctctagacttgagtttgtcacttggccccagcagaaactgttttccagatcacatattctggctgtatgtcatggcacttctccaccaaaatgctcagggaccagtcggtccagggtatgttctagagggcttttcagcatttagggggcagcagcattaggcagaacttctagatggagatctgaacagagctgaggtccaggtatagctctcctaaggttcttctttctttcctctttacagattttaaagcctgcagagaaaaagaagcccaacgccacgagacctgtcacaccaccacggggtatgatcacaaagcaagcaaagaaatagatgtcacttgacactgcctggttgggacttgtaacatagcgttcataaccttcctttttaaactgtgatgtgctggtcagcttgcccaggtagacctgtctgtcgggccctcctccatttgattactgctggcacttgctggttatagcagcaagccaagcacttcattctcaagagagcattttgttttgaacctctgttccctttgtggacagctctgatgatggtgttaagctgtacaccctggcaggttatcctgtctgaggagaaagtgtacaattgatctttttttaatttagtataagtcatgaataatgtaaatgcctgttttctttaggatataaagagagccttagagtgcgtgagtctctacatgtaattgtcataaatgcattctgttgatacaaaccactgtgaacaattttttttccagtttgtttgaaagggactgctttccctcattgtcttgtcatgtacaaactagtgtctgcagctgtggcagcaggagtgacctgcctgccgccagccctgcccagactatctgaagcacactccttcccactgcacatttaataatgattaaagccattcttttcaatgtctgtgattccttcctaaagccaaagtttctgttggactgtatggcacgccctggggatgaggtggccagggcatcgaggctgcgtgcacaggccgcctccctccgtggggcctcagaagcaggttattttaactagcaatagtggtatagtgctgagtaagctattaatgatggaagttaatgacactttgtacagttcccatatagtctattcactgagtgatctttttacagttggatcaggcctgaacccgtccattcagaaagcttcaaattatagaaacaacactgtcctatacgagtgaccgataatgctttctttggctacattctttattctgcggtgacattgaggcttataaatcaaaaggaactaacttgccgtccaccggtttatacagaactcacagtatctatgacttttttaaactacgacctgttaaatgaatctgtttgcacagatgcccgtgtacaatgccatgtgctgagaatggtttcagacttattaaatgcaagcttgttaacttg

SEQ ID NO: 13 - protein, 379 aa, Homo sapiens
MAAAAAQGGGGGEPRRTEGVGPGVPGEVEMVKGQPFDVGPRYTQLQYIGEGAYGMVSSAYDHVRKTRVAIKKISPFEHQTYCQRTLREIQILLRFRHENVIGIRDILRASTLEAMRDVYIVQDLMETDLYKLLKSQQLSNDHICYFLYQILRGLKYIHSANVLHRDLKPSNLLINTTCDLKICDFGLARIADPEHDHTGFLTEYVATRWYRAPEIMLNSKGYTKSIDIWSVGCILAEMLSNRPIFPGKHYLDQLNHILGILGSPSQEDLNCIINMKARNYLQSLPSKTKVAWAKLFPKSDSKALDLLDRMLTFNPNKRITVEEALAHPYLEQYYDPTDEPVAEEPFTFAMELDDLPKERLKELIFQETARFQPGVLEAP

SEQ ID NO: 14 - DNA, 1866 bp, Homo sapiens
cgttcctcggcgccgccggggccccagagggcagcggcagcaacagcagcagcagcagcagcgggagtggagatggcggcggcggcggctcaggggggcgggggcggggagccccgtagaaccgagggggtcggcccgggggtcccgggggaggtggagatggtgaaggggcagccgttcgacgtgggcccgcgctacacgcagttgcagtacatcggcgagggcgcgtacggcatggtcagctcggcctatgaccacgtgcgcaagactcgcgtggccatcaagaagatcagccccttcgaacatcagacctactgccagcgcacgctccgggagatccagatcctgctgcgcttccgccatgagaatgtcatcggcatccgagacattctgcgggcgtccaccctggaagccatgagagatgtctacattgtgcaggacctgatggagactgacctgtacaagttgctgaaaagccagcagctgagcaatgaccatatctgctacttcctctaccagatcctgcggggcctcaagtacatccactccgccaacgtgctccaccgagatctaaagccctccaacctgctcagcaacaccacctgcgaccttaagatttgtgatttcggcctggcccggattgccgatcctgagcatgaccacaccggcttcctgacggagtatgtggctacgcgctggtaccgggccccagagatcatgctgaactccaagggctataccaagtccatcgacatctggtctgtgggctgcattctggctgagatgctctctaaccggcccatcttccctggcaagcactacctggatcagctcaaccacattctgggcatcctgggctccccatcccaggaggacctgaattgtatcatcaacatgaaggcccgaaactacctacagtctctgccctccaagaccaaggtggcttgggccaagcttttccccaagtcagactccaaagcccttgacctgctggaccggatgttaacctttaaccccaataaacggatcacagtggaggaagcgctggctcacccctacctggagcagtactatgacccgacggatgagccagtggccgaggagcccttcaccttcgccatggagctggatgacctacctaaggagcggctgaaggagctcatcttccaggagacagcacgcttccagcccggagtgctggaggccccctagcccagacagacatctctgcaccctggggcctggacctgcctcctgcctgcccctctcccgccagactgttagaaaatggacactgtgcccagcccggaccttggcagcccaggccggggtggagcatgggcctggccacctctctcctttgctgaggcctccagcttcaggcaggccaaggccttctcctccccacccgccctccccacggggcctcgggagctcaggtggccccagttcaatctcccgctgctgctgctgctgcgcccttaccttccccagcgtcccagtctctggcagttctggaatggaagggttctggctgccccaacctgctgaagggcagaggtggagggtggggggcgctgagtagggactcagggccatgcctgcccccctcatctcattcaaaccccaccctagtttccctgaaggaacattccttagtctcaagggctagcatccctgaggagccaggccgggccgaatcccctccctgtcaaagctgtcacttcgcgtgccctcgctgcttctgtgtgtggtgagcagaagtggagctggggggcgtggagagcccggcgcccctgccacctccctgacccgtctaatatataaatatagagatgtgtctatggctgaaaaaaaaaaaaaaaa

SEQ ID NO: 15 - protein, 360 aa, Homo sapiens [sequence omitted]

SEQ ID NO: 16 - DNA, 1514 bp, Homo sapiens
gcccctccctccgcccgcccgccggcccgcccgtcagtctggcaggcaggcaggcaatcggtccgagtggctgtcggctcttcagctctcccgctcggcgtcttccttcctcctcccggtcagcgtcggcggctgcaccggcggcggcgcagtccctgcgggaggggcgacaagagctgagcggcggccgccgagcgtcgagctcagcgcggcggaggcggcggcggcccggcagccaacatggcggcggcggcggcggcgggcgcgggcccggagatggtccgcgggcaggtgttcgacgtggggccgcgctacaccaacctctcgtacatcggcgagggcgcctacggcatggtgtgctctgcttatgataatgtcaacaaagttcgagtagctatcaagaaaatcagcccctttgagcaccagacctactgccagagaaccctgagggagataaaaatcttactgcgcttcagacatgagaacatcattggaatcaatgacattattcgagcaccaaccatcgagcaaatgaaagatgtatatatagtacaggacctcatggaaacagatctttacaagctcttgaagacacaacacctcagcaatgaccatatctgctattttctctaccagatcctcagagggttaaaatatatccattcagctaacgttctgcaccgtgacctcaagccttccaacctgctgctcaacaccacctgtgatctcaagatctgtgactttggcctggcccgtgttgcagatccagaccatgatcacacagggttcctgacagaatatgtggccacacgttggtacagggctccagaaattatgttgaattccaagggctacaccaagtccattgatatttggtctgtaggctgcattctggcagaaatgctttctaacaggcccatctttccagggaagcattatcttgaccagctgaaccacattttgggtattcttggatccccatcacaagaagacctgaattgtataataaatttaaaagctaggaactatttgctttctcttccacacaaaaataaggtgccatggaacaggctgttcccaaatgctgactccaaagctctggacttattggacaaaatgttgacattcaacccacacaagaggattgaagtagaacaggctctggcccacccatatctggagcagtattacgacccgagtgacgagcccatcgccgaagcaccattcaagttcgacatggaattggatgacttgcctaaggaaaagctcaaagaactaatttttgaagagactgctagattccagccaggatacagatcttaaatttgtcaggtacctggagtttaatacagtgagctctagcaagggaggcgctgccttttgtttctagaatattatgttcctcaaggtccattattttgtattcttttccaagctccttattggaaggtatttttttaaatttagaattaaaaattatttagaaagttacatataaaaaaaaaaaaaaaaaa